(19) United States
(12) Patent Application Publication
(10) Pub. No.: US 2026/0207704 A1
(43) Pub. Date: Jul. 23, 2026

(54) STAPLED PEPTIDES AND METHODS THEREOF

(71) Applicant: Parabilis Medicines, Inc., Cambridge, MA (US)

(72) Inventors: Brian Halbert White, Malden, MA (US); Jonathan Barry Hurov, Bedford, MA (US); Yaguang Si, Arlington, MA (US); Daniel Seungduk La, Chestnut Hill, MA (US); Lorenzo Josue Alfaro-Lopez, Belmont, MA (US); Paula Cristina Ortet, Malden, MA (US); Sarah Isabelle Cappucci, Tyngsboro, MA (US); Zhi Li, Braintree, MA (US); John Hanney McGee, Somerville, MA (US); Martin Robert Tremblay, Melrose, MA (US); Gregory L. Verdine, Boston, MA (US); Kevin Ling, Somerville, MA (US); Peicheng Du, Lexington, MA (US)

(21) Appl. No.: 19/139,189

(22) PCT Filed: Dec. 15, 2023
(86) PCT No.: PCT/US2023/084461

§ 371 Date: Jun. 13, 2025

Related U.S. Application Data
(60) Provisional application No. 63/432,998, filed on Dec. 15, 2022.
(60) Provisional application No. 63/459,636, filed on Apr. 15, 2023.

(51) Int. Cl. A61K 38/12; A61K 45/06; A61P 35/00

(52) U.S. Cl. CPC A61K 38/12; A61K 45/06; A61P 35/00

(57) ABSTRACT

Among other things, the present disclosure provides various useful agents. In some embodiments, provided agents can bind to beta-catenin. In some embodiments, the present disclosure provides technologies for modulating beta-catenin functions. In some embodiments, the present disclosure provides technologies for preventing and/or treating conditions, disorders or diseases associated with beta-catenin. In some embodiments, the present disclosure provides designed amino acids and agents which can provide improved properties and/or activities.

### CROSS-REFERENCE TO RELATED APPLICATIONS

This application claims priority to U.S. Provisional Application No. 63/432,998, filed Dec. 15, 2022, and U.S. Provisional Application No. 63/459,636, filed Apr. 15, 2023, the entirety of each of which is incorporated herein by reference.

### BACKGROUND

Stapled peptides are useful for various applications. For example, as biologically active agents, they can be utilized to modulate various biological functions.

### SUMMARY

Among other things, the present disclosure provides powerful technologies (e.g., agents (e.g., those that are or comprise peptides, in many embodiments, stapled peptides), compositions, methods, etc.) for modulating various biological functions. In some embodiments, provided technologies are useful for treating hepatocellular cancer, e.g., those comprising a beta-catenin mutation such as D32H, D32V, S37C, S45F, etc.

In some embodiments, the present disclosure provides agents, e.g., stapled peptides that comprise multiple staples. In some embodiments, the present disclosure provides agents, e.g., stapled peptides that comprise three or more staples. In some embodiments, the present disclosure provides agents, e.g., stapled peptides that comprise three or more staples within 10-20 amino acid residues, e.g., 10-15, 11-15, 11-14, 11, 12, 13, 14, 15, 16, 17, 18, 19, or 20 consecutive amino acid residues. In some embodiments, the present disclosure provides agents, e.g., stapled peptides that comprise three or more staples within 11 consecutive amino acid residues. In some embodiments, the present disclosure provides agents, e.g., stapled peptides that comprise three or more staples within 14 consecutive amino acid residues. In some embodiments, within such numbers of amino acid residues there are three staples. In some embodiments, within such numbers of consecutive amino acid residues there are four staples. Without the intention to be limited by theory, in some embodiments, provided agents, e.g., stapled peptides have increased rigidity than reference peptides (e.g., unstapled peptides, or stapled peptides having fewer staples (in some embodiments, fewer staples within certain numbers of amino acid residues as described herein), etc.). In some embodiments, provided agents, e.g., stapled peptides demonstrate various desired properties and/or activities. In some embodiments, provided agents, e.g., stapled peptides provide improved desired properties and/or activities than reference peptides (e.g., unstapled peptides, or stapled peptides having fewer staples (in some embodiments, fewer staples within certain numbers of amino acid residues as described herein), etc.).

In some embodiments, provided technologies comprise designed structural features, e.g., novel amino acid residues, that can provide significantly improved properties and/or activities compared to comparable reference technologies that do not contain such designed structural features. In some embodiments, the present disclosure provides designed amino acids as described herein, whose incorporation into peptide agents, including stapled peptides, can provide significantly improved properties and/or activities such as improved lipophilicity and/or delivery into cells compared to reference amino acids (e.g., Asp). In some embodiments, the present disclosure provides technologies including peptides comprising such designed amino acid residues. In some embodiments, the present disclosure provides stapled peptides comprise such designed amino acid residues.

In some embodiments, the present disclosure provides technologies for modulating one or more functions of beta-catenin. Particularly, in some embodiments, the present disclosure provides various agents, e.g., peptides, in many instances stapled peptides, that can bind to beta-catenin and modulate its functions. As demonstrated herein, in some embodiments, the present disclosure binds agents that can interact with beta-catenin at a unique set of residues. In some embodiments, a binding site comprises one or more or all of the set of residues. In some embodiments, provided agents interact with one or more of a set of residues that are or correspond to the following residues of SEQ ID NO: 1: A305, Y306, G307, N308, Q309, K312, R342, K345, V346, V349, Q375, R376, Q379, N380, L382, W383, R386, N387, D413, N415, V416, T418, and C419. In some embodiments, provided agents interact with one or more of amino acid residue that are or correspond to A305, Y306, G307, N308, Q309, K312, R342, K345, V346, V349, Q375, Q379, N380, L382, W383, R386, N387, D413, N415, V416, T418, and C419 of SEQ ID NO: 1. In some embodiments, provided agents interact with one or more of amino acid residues that are or correspond to A305, Y306, G307, N308, Q309, K312, K345, V346, V349, Q379, N380, L382, W383, R386, N387, D413, N415, V416, T418, and C419 of SEQ ID NO: 1. In some embodiments, provided agents interact with one or more of amino acid residues that are or correspond to G307, K312, K345, W383, N387, D413, and N415 of SEQ ID NO: 1. In some embodiments, provided agents interact with one or more of amino acid residues that are or correspond to K312, K345, R386 and W383 of SEQ ID NO: 1. In some embodiments, provided agents interact with one or more of a set of residues that are or correspond to the following residues of SEQ ID NO: 1: G307, K312, K345, Q379, L382, W383, N387, N415, and V416. In some embodiments, provided agents interact with all of a set of residues that are or correspond to the following residues of SEQ ID NO: 1: Y306, G307, K312, K345, Q379, L382, W383, N387, N415, and V416. In some embodiments, provided agents interact with all of a set of residues that are or correspond to the following residues of SEQ ID NO: 1: G307, K312, K345, Q379, L382, W383, N387, N415, and V416. In some embodiments, provided agents interact with all of a set of residues that are or correspond to the following residues of SEQ ID NO: 1: Y306, G307, K312, K345, Q379, L382, W383, N387, N415, and V416. In some embodiments, provided agents interact with one or more of amino acid residues that are or correspond to K312, K345 and W383 of SEQ ID NO: 1. In some embodiments, provided agents interact with the amino acid residues that are or correspond to K312, K345 and W383 of SEQ ID NO: 1.

As demonstrated herein, provided technologies can modulate one or more biological processes associated with beta-catenin. In some embodiments, provided agents, e.g., stapled peptides, compete with a ligand (e.g., with a member of the T cell factor/lymphoid enhancer factor (TCF/LEF) family of transcription factors) for binding to beta-catenin. In some embodiments, provided agents compete with a ligand for binding to beta-catenin at a particular binding site (e.g., with a member of the T cell factor/lymphoid enhancer factor (TCF/LEF) family of transcription factors at the TCF site on beta-catenin). In some embodiments, provided technologies compete with TCF for interactions with beta-catenin. In some embodiments, binding of provided agents to a beta-catenin site decreases, suppresses and/or blocks binding to beta-catenin by another binding partner (e.g., a kinase). In some embodiments, binding of provided agents blocks binding of beta-catenin by a TCF/LEF family member. In some embodiments, the present disclosure provides agents that can bind to a site of beta-catenin selectively over one of more other binding sites by other ligands (e.g., peptides, proteins, etc.; in some embodiments, a ligand is Axin; in some embodiments, a ligand is Bcl9). In some embodiments, provided technologies modulate one or more beta-catenin functions associated with its interactions with TCF. In some embodiments, provided technologies selectively modulate beta-catenin functions, e.g., functions associated with TCF interactions. In some embodiments, provided technologies selectively modulate beta-catenin functions and do not significantly impact functions that are not associated with beta-catenin (e.g., various functions and/or processes in the Wnt pathway that are not associated with beta-catenin). In some embodiments, provided technologies are useful for inhibiting beta-catenin functions. In some embodiments, provided technologies are usefully for promoting and/or enhancing immune activities, e.g., anti-tumor adaptive immunity.

In some embodiments, provided technologies are useful for preventing or treating various conditions, disorders or diseases including cancer. In some embodiments, the present disclosure provides methods for treating or preventing a condition, disorder or disease associated with beta-catenin, comprising administering to a subject suffered therefrom or susceptible thereto an effective amount of a provided agent or a pharmaceutically acceptable salt thereof. In some embodiments, a condition, disorder or disease is associated with beta-catenin's interactions with TCF. In some embodiments, a condition, disorder or disease is hepatocellular cancer.

In some embodiments, an agent, e.g., a staple peptide, is administered as a pharmaceutical composition. In some embodiments, the present disclosure provides pharmaceutical compositions which comprise or deliver a provided agent or a pharmaceutically acceptable salt thereof. In some embodiments, a pharmaceutical composition further comprises a lipid. As demonstrated herein, in some embodiments, a suitable lipid can promote delivery/activities. In some embodiments, an agent is or comprises a peptide. In some embodiments, an agent is or comprises a stapled peptides. In some embodiments, provided agents that can bind beta-catenin comprise one or more designed amino acid residues. In some embodiments, the present disclosure provides a pharmaceutical composition comprising an effective amount of an agent, e.g., a stapled peptide, wherein when the composition is administered to a model as described herein, a result as described herein (e.g., reduction of tumor size, inhibition of tumor growth, etc.) is observed. In some embodiments, provided technologies, e.g., agents, compositions, etc., are capable of reducing hepatocellular tumor growth, or shrinking hepatocellular tumor size, e.g., in a subject, a PDX model, etc. In some embodiments, they are capable of reducing hepatocellular cancer cell proliferation. In some embodiments, a hepatocellular tumor or hepatocellular cancer cell comprises a mutation, e.g., a mutation in beta-catenin such as D32H, D32V, S37C, S45F, etc.

In some embodiments, the present disclosure provides agents that bind to a polypeptide comprising or consisting of SEQ ID NO: 1 (Uniprot ID P35222), or residues 250-450 of SEQ ID NO: 1, or residues 305-419 of SEQ ID NO: 1:

Uniprot No. P35222
(SEQ ID NO: 1)
MATQADLMELDMAMEPDRKAAVSHWQQQSYLDSGIHSGATTTAPS
LSGKGNPEEEDVDTSQVLYEWEQGFSQSFTQEQVADIDGQYAMTR
AQRVRAAMFPETLDEGMQIPSTQFDAAHPTNVQRLAEPSQMLKHA
VVNLINYQDDAELATRAIPELTKLLNDEDQVVVNKAAVMVHQLSK
KEASRHAIMRSPQMVSAIVRTMQNTNDVETARCTAGTLHNLSHHR
EGLLAIFKSGGIPALVKMLGSPVDSVLFYAITTLHNLLLHQEGAK
MAVRLAGGLQKMVALLNKTNVKFLAITTDCLQILAYGNQESKLII
LASGGPQALVNIMRTYTYEKLLWTTSRVLKVLSVCSSNKPAIVEA
GGMQALGLHLTDPSQRLVQNCLWTLRNLSDAATKQEGMEGLLGTL
VQLLGSDDINVVTCAAGILSNLTCNNYKNKMMVCQVGGIEALVRT
VLRAGDREDITEPAICALRHLTSRHQEAEMAQNAVRLHYGLPVVV
KLLHPPSHWPLIKATVGLIRNLALCPANHAPLREQGAIPRLVQLL
VRAHQDTQRRTSMGGTQQQFVEGVRMEEIVEGCTGALHILARDVH
NRIVIRGLNTIPLFVQLLYSPIENIQRVAAGVLCELAQDKEAAEA
IEAEGATAPLTELLHSRNEGVATYAAAVLFRMSEDKPQDYKKRLS
VELTSSLFRTEPMAWNETADLGLDIGAQGEPLGYRQDDPSYRSFH
SGGYGQDALGMDPMMEHEMGGHHPGADYPVDGLPDLGHAQDLMDG
LPPGDSNQLAWFDTDL.

In some embodiments, provided agents specifically interact with one or more residues which are or correspond to residues 305-419 of SEQ ID NO: 1. In some embodiments, provided agents bind to a motif (e.g., a portion of a polypeptide, a domain of a polypeptide, etc.) that comprise one or more residues corresponding to Ala305, Tyr306, Gly307, Asn 308, Gln309, Lys312, Arg342, Lys345, Val346, Val349, Gln375, Arg376, Gln379, Asn380, Leu382, Trp383, Arg386, Asn387, Asp413, Asn415, Val416, Thr418, and Cys419 of SEQ ID NO: 1. In some embodiments, provided agents bind to a motif (e.g., a portion of a polypeptide, a domain of a polypeptide, etc.) that comprise one or more residues corresponding to Ala305, Tyr306, Gly307, Asn 308, Gln309, Lys312, Lys345, Val346, Val349, Gln375, Arg376, Gln379, Asn380, Lcu382, Trp383, Arg386, Asn387, Asp413, Asn415, Val416, Thr418, and Cys419 of SEQ ID NO: 1. In some embodiments, an agent binds to a motif comprising one or more of the following residues within SEQ ID NO: 1: Ala305, Tyr306, Gly307, Asn 308, Gln309, Lys312, Arg342, Lys345, Val346, Val349, Gln375, Arg376, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Lys345, Val346, Val349, Gln375, Arg376, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Arg342, Lys345, Val346, Val349, Gln 375, Gln379, Asn380, Leu382, Trp383, Arg386, Asn387, Asp413, Asn415, Val416, Thr418, and Cys419. In some embodiments, an agent binds to a motif comprising one or more of the following residues within SEQ ID NO: 1: Ala305, Tyr306, Gly307, Asn 308, Gln309, Lys312, Lys345, Val346, Val349, Gln379, Asn380, Leu382, Trp383, Arg386, Asn387, Asp413, Asn415, Val416, Thr418, and Cys419. In some embodiments, provided technologies bind to a motif comprising at least 2, 3, 4, 5, or 6 of G307, K312, K345, W383, N387, and N415. In some embodiments, provided technologies bind to a motif comprising at least 2, 3, 4, 5, 6, or 7 of G307, K312, K345, W383, N387, D413, and N415. In some embodiments, provided agents specifically bind to such motifs. In some embodiments, a motif may be referred to as a binding site. In some embodiments, provided technologies selectively bind to such a binding site over an Axin binding site. In some embodiments, provided technologies selectively bind to such a binding site over a Bc19 binding site. In some embodiments, provided technologies selectively bind to such a binding site over an ICAT binding site. In some embodiments, provided technology binds to such a binding site in a reverse N to C direction compared to TCF. In some embodiments, provided technologies do not bind to Axin binding site of beta-catenin. In some embodiments, provided technologies do not bind to Bcl9 binding site of beta-catenin. In some embodiments, provided technologies do not bind to ICAT binding site of beta-catenin. Various technologies, e.g., crystallography, NMR, biochemical assays, etc., may be utilized to assess interactions with beta-catenin in accordance with the present disclosure.

In some embodiments, the provided technology provides an agent, e.g., a stapled peptide, that comprises multiple, e.g., two, three, or more, staples within 10-20, 10-15, 10, 11, 12, 13, 14, 15, 16, 17, 18, 19, or 20 consecutive amino acids residues. In some embodiments, there are three or more staples within 10 consecutive amino acid residues. In some embodiments, there are three or more staples within 11 consecutive amino acid residues. In some embodiments, there are three or more staples within 12 consecutive amino acid residues. In some embodiments, there are three or more staples within 13 consecutive amino acid residues. In some embodiments, there are three or more staples within 14 consecutive amino acid residues. In some embodiments, there are three or more staples within 15 consecutive amino acid residues. In some embodiments, there are three or more staples within 16 consecutive amino acid residues. In some embodiments, there are three or more staples within 17 consecutive amino acid residues. In some embodiments, there are three or more staples within 18 consecutive amino acid residues. In some embodiments, there are three or more staples within 19 consecutive amino acid residues. In some embodiments, there are three or more staples within 20 consecutive amino acid residues. In some embodiments, two staples are bonded to the same amino acid residue. In some embodiments, two staples are bonded to the same backbone atom. In some embodiments, two staples are bonded to the same backbone carbon atom. In some embodiments, two staples are bonded to an alpha-carbon atom of an amino acid residue, and each independently bonds to another amino acid residue.

In some embodiments, a first staple in an agent, e.g., a staple peptide, are bonded to amino acid residues at positions i and i+3. In some embodiments, there is a second staple bonded to amino acid residues at positions i+3 and i+10. In some embodiments, there a third staple bonded to amino acid residues at positions i+9 and i+13. Those skilled in the art appreciate that as used in the art, i, i+3, i+9, i+10, i+13, etc. are routinely utilized to indicate relevant positions of amino acid residues. In some embodiments, they may also indicate absolute positions in an agent, e.g., a peptide. In some embodiments, i is an integer of 1-50 (e.g., 1, 2, 3, 4, 5, 6, 7, 8, 9, 10, 11, 12, 13, 14, 15, 16, 17, 18, 19, or 20). In some embodiments, i is 1. In some embodiments, there is a fourth staple in an agent, e.g., a stapled peptide.

In some embodiments, there are two amino acid residues between two amino acid residues bonded to the same staple. Such a staple may be referred to as a (i, i+3) staple. Similarly, in some embodiments, there are 3, 4, 5, 6, 7, 8, 9, or 10 amino acid residues between two amino acid residues bonded to the same staple, and such a staple may be referred to as a (i, i+4), (i, i+5), (i, i+6), (i, i+7), (i, i+8), (i, i+9), (i, i+10), or (i, i+11) staple, respectively.

In some embodiments, an agent, e.g., a stapled peptide, comprises a (i, i+2) staple and a (i, i+7) staple. In some embodiments, an agent, e.g., a stapled peptide, comprises a (i, i+3) staple and a (i, i+7) staple. In some embodiments, a (i, i+3) staple and (i, i+7) staple are bonded to the same amino acid residue. In some embodiments, a (i, i+3) staple and (i, i+7) staple bond to the same atom. In some embodiments, a (i, i+3) staple and (i, i+7) staple bond to the same alpha carbon atom. For example, in compound I-1, a (i, i+3) staple is bonded to amino acid residues at positions 1 and 4, and a (i, i+7) staple is bonded to amino acid residues at positions 4 and 11, and the two staples are both bonded to the alpha carbon of the amino acid residue at position 4. In some embodiments, an agent further comprises a third staple. In some embodiments, a third staple is (i, i+4). In some embodiments, a third staple is (i, i+7). In some embodiments, a third staple is not bonded to any of the amino acid residues that are bonded to the first two staples. In some embodiments, an agent further comprises a fourth staple. In some embodiments, a fourth staple is (i, i+4). In some embodiments, a fourth staple is (i, i+7). In some embodiments, a fourth staple is not bonded to any of the amino acid residues that are bonded to the first two staples. In some embodiments, a fourth staple is not bonded to any of the amino acid residues that are bonded to the first third staples.

In some embodiments, a provided agent, e.g., a peptide agent such as a stapled peptide agent, comprises one or more (e.g., 1, 2, 3, 4, 5, 6, or 7) of the following groups (in some embodiments, from the N to C direction):
a first acidic group (e.g., of a first acidic amino acid residue);
a second acidic group (e.g., of a second acidic amino acid residue);
optionally a third acidic group (e.g., of a third acidic amino acid residue);
optionally a hydrophobic group (e.g., of a hydrophobic amino acid residue)
a first aromatic group (e.g., of a first aromatic amino acid residue);
a second aromatic group (e.g., of a first aromatic amino acid residue); and
a third aromatic group (e.g., of a third aromatic amino acid residue).

In some embodiments, an agent comprises a first and second acidic group and a first, second and third aromatic group. In some embodiments, such an agent additionally comprises a third acidic group (e.g., of a third acid amino acid residue) and/or a hydrophobic group (e.g., of a hydrophobic amino acid residue). In some embodiments, such an agent additionally comprises a third acidic group (e.g., of a third acid amino acid residue) and a hydrophobic group (e.g., of a hydrophobic amino acid residue). In some embodiments, the distance between a first acidic group and a second acidic group is about the distance between the acidic groups of two acidic amino acid residues of a peptide motif, wherein there are two amino acid residues between the two acidic amino acid residues (e.g., if the first acidic amino acid residue is at position N, the second is at position N+3), the distance between a first acidic group and a third acidic group (if present) is about the distance between the acidic groups of two acidic amino acid residues of a peptide motif, wherein there are three amino acid residues between the two acidic amino acid residues (e.g., if the first acidic amino acid residue is at position N, the third is at position N+4), the distance between a first acidic group and a hydrophobic group (if present) is about the distance between the acidic group of an acidic amino acid residue and the hydrophobic group of a hydrophobic amino acid residue of a peptide motif, wherein there are five amino acid residues between the first acidic amino acid residue and the hydrophobic amino acid residue (e.g., if the first acidic amino acid residue is at position N, the hydrophobic amino acid residue is at position N+6), the distance between a first acidic group and a first aromatic group is about the distance between the acidic group of a first acidic amino acid residue and the aromatic group of an aromatic amino acid residue of a peptide motif, wherein there are six amino acid residues between the first acidic amino acid residue and the first aromatic amino acid residue (e.g., if the first acidic amino acid residue is at position N, the first aromatic amino acid residue is at position N+7), the distance between the first aromatic group and the second aromatic group is about the distance between the aromatic groups of two aromatic amino acid residues of a peptide motif, wherein there are two amino acid residues between the two aromatic amino acid residues (e.g., if the first aromatic amino acid residue is at position M, the second is at position M+3), and/or the distance between the first aromatic group and the third aromatic group is about the distance between the aromatic groups of two aromatic amino acid residues of a peptide motif, wherein there are three amino acid residues between the two aromatic amino acid residues (e.g., if the first aromatic amino acid residue is at position M, the third is at position M+4). In some embodiments, a first acidic amino acid residue is at position N, a second acidic amino acid residue is at position N+3, and a first, second and third aromatic amino acid residue are at positions N+7, N+10 and N+11, respectively. In some embodiments, a first acidic amino acid residue is at position N, a second acidic amino acid residue is at position N+3, a third acidic amino acid residue is at position N+4, and a first, second and third aromatic amino acid residue are at positions N+7, N+10 and N+11, respectively. In some embodiments, a first acidic amino acid residue is at position N, a second acidic amino acid residue is at position N+3, a hydrophobic amino acid residue is at position N+6, and a first, second and third aromatic amino acid residue are at positions N+7, N+10 and N+11, respectively. In some embodiments, a first acidic amino acid residue is at position N, a second acidic amino acid residue is at position N+3, a third acidic amino acid residue is at position N+4, a hydrophobic amino acid residue is at position N+6, and a first, second and third aromatic amino acid residue are at positions N+7, N+10 and N+11, respectively. In some embodiments, M is N+7. In some embodiments, N is 1-7. In some embodiments, Nis 1, 2, 3, 4, or 5. In some embodiments, N is 1. In some embodiments, N is 2. In some embodiments, N is 3. In some embodiments, N is 4. In some embodiments, N is 5. In some embodiments, M is 8-16. In some embodiments, M is 8. In some embodiments, M is 9. In some embodiments, M is 10. In some embodiments, M is 11. In some embodiments, M is 12. In some embodiments, M is 13. In some embodiments, a peptide motif is an alpha-helical motif wherein each amino acid residue is independently an alpha amino acid residue. In some embodiments, a peptide motif is stapled. In some embodiments, there are two or more staples in a peptide motif; in some embodiments, there are three; in some embodiments, there are four; in some embodiments, there are four or more. In some embodiments, a peptide motif is or comprises an agent described in a Table herein (e.g., I-xxxx wherein xxxx is a number (e.g., I-1, I-10, I-100, I-1000, etc.)). In some embodiments, a first acidic group is of X² as described herein, a second acidic group is of X⁵ as described herein, a third acidic group (if present) is of X⁶ as described herein, a hydrophobic group (if present) is of X⁸ as described herein, a first aromatic group is of X⁹ as described herein, a second aromatic group is of X¹² as described herein, and/or a third aromatic group is of X¹³ as described herein. In some embodiments, as described herein, a provided agent is a stapled peptide comprising one or more staples. In some embodiments, as described herein, a provided agent is a stapled peptide comprising two or more staples. In some embodiments, as described herein, a provided agent is a stapled peptide comprising three or more staples. In some embodiments, when contacted with a beta-catenin polypeptide, a first acidic group interacts with Lys312 and/or Gly307 or amino acid residues corresponding thereto, a second acidic group interacts with Asn387, Trp383 and/or Arg386 or amino acid residues corresponding thereto, a first aromatic group interacts with Lys345 and/or Trp383 or amino acid residues corresponding thereto, a second aromatic group interacts with Trp383 and/or Asn415 or amino acid residues corresponding thereto, and a third aromatic group interacts with Gln379, Leu383, Val416, Asn415 and/or Trp383 or amino acid residues corresponding thereto. In some embodiments, a third acidic group interacts with Asn387, Trp383 and/or Arg386 or amino acid residues corresponding thereto. In some embodiments, a hydrophobic group interacts with Trp383 or an amino acid residue corresponding thereto.

In some embodiments, the present disclosure provides an agent having the structure of formula I:

or a salt thereof, wherein each variable is independently as described herein.

In some embodiments, the present disclosure provides an agent which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent which is or comprises a peptide comprising:

wherein:
each of p15, p16 and p17 is independently 0 or 1;
each of p and p′ is independently 0-10;
each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, an agent is R^{N}_[X]pX¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X]p′-R^{C}, wherein each variable is independently as described herein.

In some embodiments, an agent is or comprises X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]ₚ₁₈[X¹⁹]ₚ₁₉[X²⁰]ₚ₂₀[X²¹]ₚ₂₁[X²²]ₚ₂₂[X²³]_{p 23}, wherein each of p14, p15, p16, p17, p18, p19, p20, p21, p22, and p23 is independently 0 or 1, and each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue as described herein.

In some embodiments, such a peptide comprises three or more staples. In some embodiments, such a peptide comprises five or more residues suitable for stapling.

In some embodiments, the present disclosure provides an agent, wherein the agent is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled.

In some embodiments, the present disclosure provides an agent, wherein the agent is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X⁶ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled.

In some embodiments, an agent is or comprises a peptide. In some embodiments, an agent is or comprises a stapled peptide. In some embodiments, an agent is a peptide. In some embodiments, an agent is a stapled peptide. In some embodiments, an agent, a peptide, or a stapled peptide has the structure of [X⁰]ₚ₀X¹X²X³X⁴X⁵X⁶X⁷X⁸X⁹X¹⁰X¹X¹²X¹³X¹⁴[X¹⁵]ₚ₁₅[X¹⁶]ₚ₁₆[X¹⁷]ₚ₁₇. In some embodiments, X¹ and X⁴ and/or X⁴ and X¹¹ are independently amino acid residues suitable for stapling, or are stapled, or X³ and X¹⁰ independently amino acid residues suitable for stapling, or are stapled. In some embodiments, X¹ and X⁴ are independently amino acid residues suitable for stapling. In some embodiments, X¹ and X⁴ are stapled. In some embodiments, X⁴ and X¹¹ are independently amino acid residues suitable for stapling. In some embodiments, X⁴ and X¹¹ are stapled. In some embodiments, X¹ and X⁴, and X⁴ and X¹¹ are independently amino acid residues suitable for stapling. In some embodiments, a stapled peptide is a stitched peptide comprising two or more staples, some of which may bond to the same backbone atom. In some embodiments, X¹ and X⁴ are stapled, and X⁴ and X¹¹ are stapled. In some embodiments, a staple connecting X¹ and X⁴ and a staple connecting X⁴ and X¹¹ are bonded to a common backbone atom of X⁴. In some embodiments, a common backbone atom is the alpha-carbon of X⁴. In some embodiments, X³ and X¹⁰ are independently amino acid residues suitable for stapling. In some embodiments, X³ and X¹⁰ are stapled. In some embodiments, X¹ and X³ are independently amino acid residues suitable for stapling. In some embodiments, X¹ and X³ are stapled. In some embodiments, X¹⁰ and X¹⁴ are independently amino acid residues suitable for stapling. In some embodiments, X¹⁰ and X¹⁴ are stapled. In some embodiments, X⁷ and X¹⁰ are independently amino acid residues suitable for stapling. In some embodiments, X⁷ and X¹⁰ are stapled. In some embodiments, X⁷ and X¹⁴ are independently amino acid residues suitable for stapling. In some embodiments, X⁷ and X¹⁴ are stapled. In some embodiments, X³ and X⁷ are independently amino acid residues suitable for stapling. In some embodiments, X³ and X⁷ are stapled.

In some embodiments, the present disclosure provides agents that bind to a polypeptide comprising or consisting of residues 305-419 of SEQ ID NO: 1 as described herein. In some embodiments, an agent, e.g., a peptide, has a molecular mass of no more than about 5000 Daltons. In some embodiments, it is no more than about 2500, 3000, 3500, 4000, 4500 or 5000 Daltons. In some embodiments, it is no more than about 2500 Daltons. In some embodiments, it is no more than about 3000 Daltons. In some embodiments, it is no more than about 3500 Daltons. In some embodiments, it is no more than about 4000 Daltons. In some embodiments, it is no more than about 500 Daltons.

In some embodiments, the present disclosure provides various technologies, e.g., reagents, methods, etc., for preparing, characterizing, assessing and using provided agents and compositions thereof. In some embodiments, the present disclosure provides, e.g., methods, reagents and/or systems for identifying, characterizing and/or assessing provided agents and use thereof (e.g., as therapeutic or diagnostic agents).

In some embodiments, the present disclosure provides pharmaceutical compositions comprising or delivering a provided agent and a pharmaceutical acceptable carrier. In some embodiments, a provided agent is a pharmaceutically acceptable salt form. In some embodiments, a provided composition comprises a pharmaceutically acceptable salt form an agent. In some embodiments, in various compositions and methods, agents are provided as pharmaceutically acceptable salt forms.

In some embodiments, the present disclosure provides methods for modulating a property, activity and/or function of beta-catenin, comprising contacting beta-catenin with a provided agent. In some embodiments, the present disclosure provides methods for modulating a property, activity and/or function of beta-catenin in a system comprising or expressing beta-catenin, comprising administering or delivering to a system an effective amount of a provided agent. In some embodiments, an activity or function of beta-catenin is inhibited or reduced. In some embodiments, a property, activity and/or function is associated with beta-catenin/TCF interaction.

In some embodiments, the present disclosure provides methods for modulating beta-catenin/TCF interaction, comprising contacting beta-catenin with a provided agent. In some embodiments, the present disclosure provides methods for modulating beta-catenin/TCF interaction in a system comprising or expressing beta-catenin and TCF, comprising administering or delivering to the system an effective amount a provided agent. In some embodiments, interactions between beta-catenin and TCF is reduced. In some embodiments, interactions between beta-catenin and TCF is inhibited.

In some embodiments, the present disclosure provides methods for inhibiting cell proliferation, comprising administering or delivering to a population of cells an effective amount of a provided agent. In some embodiments, the present disclosure provides methods for inhibiting cell proliferation in a system, comprising administering or delivering to the system an effective amount of a provided agent. In some embodiments, the present disclosure provides methods for inhibiting cell growth, comprising administering or delivering to a population of cells an effective amount of a provided agent. In some embodiments, the present disclosure provides methods for inhibiting cell growth in a system, comprising administering or delivering to the system an effective amount of a provided agent. In some embodiments, such cell proliferation is beta-catenin dependent. In some embodiments, such cell growth is beta-catenin dependent. In some embodiments, such proliferation or growth is dependent on beta-catenin interactions with TCF.

In some embodiments, a system is in vitro. In some embodiments, a system is ex vivo. In some embodiments, a system is in vivo. In some embodiments, a system is or comprise a cell. In some embodiments, a system is or comprises a tissue. In some embodiments, a system is or comprises an organ. In some embodiments, a system is or comprises an organism. In some embodiments, a system is an animal. In some embodiments, a system is human. In some embodiments, a system is or comprises cells, tissues or organs associated with a condition, disorder or disease. In some embodiments, a system is or comprises cancer cells. In some embodiments, a system comprises a mutation as described herein.

In some embodiments, the present disclosure provides methods for treating conditions, disorders or diseases. In some embodiments, the present disclosure provides methods for treating a condition, disorder or disease, comprising administering or delivering to a subject suffering therefrom an effective amount of an agent of the present disclosure. In some embodiments, a symptom is reduced, removed or prevented. In some embodiments, one or more parameters for assessing a condition, disorder or disease are improved. In some embodiments, survivals of subjects are extended. As appreciated by those skilled in the art, in some embodiments, prevention, reduced risks, and/or effects of treatment may be assessed through clinical trials and may be observed in subject populations. In some embodiments, a condition, disorder or disease is cancer. In some embodiments, a condition, disorder or disease is associated with beta-catenin. In some embodiments, a condition, disorder or disease is associated with beta-catenin interaction with TCF. In some embodiments, a condition, disorder or disease is bladder cancer. In some embodiments, a condition, disorder or disease is endometrial cancer. In some embodiments, a condition, disorder or disease is adrenocortical carcinoma. In some embodiments, a condition, disorder or disease is gastric cancer. In some embodiments, a condition, disorder or disease is lung cancer. In some embodiments, a condition, disorder or disease is melanoma. In some embodiments, a condition, disorder or disease is esophageal cancer. In some embodiments, a condition, disorder or disease is colorectal cancer. In some embodiments, a cancer is liver cancer. In some embodiments, a cancer is prostate cancer. In some embodiments, a cancer is breast cancer. In some embodiments, a cancer is endometrial cancer.

In some embodiments, agents are administered as pharmaceutically compositions that comprise or deliver such agents. In some embodiments, agents are provided and/or delivered in pharmaceutically acceptable salt forms. In some embodiments, in a composition (e.g., a liquid composition of certain pH) an agent may exist in various forms including various pharmaceutically acceptable salt forms.

In some embodiments, a provided agent is utilized in combination with a second therapy. In some embodiments, a provided agent is utilized in combination with a second therapeutic agent. In some embodiments, a second therapy or therapeutic agent is administered prior to an administration or delivery of a provided agent. In some embodiments, a second therapy or therapeutic agent is administered at about the same time as an administration or delivery of a provided agent. In some embodiments, a second therapy or therapeutic agent is administered subsequently to an administration or delivery of a provided agent. In some embodiments, a subject is exposed to both a provided agent and a second therapeutic agent. In some embodiments, a subject is exposed to a therapeutic effect of a provided agent and a therapeutic effect of a second therapeutic agent. In some embodiments, a second therapy is or comprises surgery. In some embodiments, a second therapy is or comprises radiation therapy. In some embodiments, a second therapy is or comprises immunotherapy. In some embodiments, a second therapeutic agent is or comprises a drug. In some embodiments, a second therapeutic agent is or comprises a cancer drug. In some embodiments, a second therapeutic agent is or comprises a chemotherapeutic agent. In some embodiments, a second therapeutic agent is or comprises a hormone therapy agent. In some embodiments, a second therapeutic agent is or comprises a kinase inhibitor. In some embodiments, a second therapeutic agent is or comprises a checkpoint inhibitor (e.g., antibodies against PD-1, PD-L1, CTLA-4, etc.). In some embodiments, a provide agent can be administered with lower unit dose and/or total dose compared to being used alone. In some embodiments, a second agent can be administered with lower unit dose and/or total dose compared to being used alone. In some embodiments, one or more side effects associated with administration of a provided agent and/or a second therapy or therapeutic agent are reduced. In some embodiments, a combination therapy provides improved results, e.g., when compared to each agent utilized individually. In some embodiments, a combination therapy achieves one or more better results, e.g., when compared to each agent utilized individually.

Further description of certain embodiments of provided technologies is presented below.

### BRIEF DESCRIPTION OF THE DRAWING

FIG. 1. Provided technologies can inhibit beta-catenin driven gene transcription selectively in cells expressing beta-catenin. Stapled peptides inhibited endogenous gene expression in wild HAP1 isogenic cell but not in CTNNB1 knockout (KO) cells. (A): beta-catenin levels. CHIR: CHIR99021, which can activate beta-catenin pathway and increase AXIN2 and SP5 expression. (B): SP5 expression (24h). (C): AXIN2 expression (24h). For each group, from left to right, DMSO (“0” and “0”), Peptide A (1 and 5 uM), I-66 (1 and 5 uM) and I-470 (1 and 5 uM). Expression assessed after 24 hour treatment.

FIG. 2. Provided technologies can reduce nuclear beta-catenin levels. Results for total beta-catenin in nuclear fraction (24 h) are shown as examples.

FIG. 3. Provided technologies can inhibit cell proliferation, modulate transcription and/or induce cell cycle arrest. (A): Provided technologies can reduce cell proliferation. (B) and (C): Provided technologies can modulate gene expression. (B): AXIN 24 hr. (C): CXCL12 24 hr. (D): Provided technologies can induce cell cycle arrest. For left to right: Peptide A (1, 5 and 10 uM), I-66 (1, 5 and 10 uM), I-470 (1, 5 and 10 uM) and DMSO.

FIG. 4. Provided technologies can provide robust, dose-dependent anti-tumor effects in vivo. Both dose levels assessed provided robust reduction of tumor sizes, and the higher dose levels provided greater reductions. COLO320DM cells (colon cancer, mutations: APC, TP53) were utilized for the presented data. Top line is for vehicle treatment, the middle line is for I-66, 30 mg/kg, Q4D, and the bottom line is for I-66, 75 mg/kg, Q4D.

FIG. 5. Provided technologies can provide sustained tumor exposure, suitable pharmacokinetic profiles and broad tissue distribution. (A): Sustained COLO320DM xenograft tumor exposure after a single i.p. injection of I-66 at 50 mg/kg was shown as an example. Dotted line indicates in vitro proliferation IC₅₀ (0.7 uM). (B): Mouse plasma pharmacokinetics. Data presented are I-66 plasma concentration (ng/ml) over time as examples. (C): Tissue distribution observed for I-66 in one assessment. Mouse single dose IP, 50 mg/kg. For each sample, the left column is 24 h data and the right is 96 h data.

FIG. 6. ¹H NMR of a preparation of I-66 prepared as described in Example 3 (DMSO-d6, 373K).

FIG. 7. Integration of peaks in a ¹H NMR spectrum of a preparation of I-66 prepared as described in Example 3 (DMSO-d6, 373K). Those skilled in the art appreciate that integration may be further adjusted and/or optimized.

FIG. 8. Provided technologies can provide robust anti-tumor effects in vivo in multiple tumor models. (A): Certain data from a colorectal (CRC) patient derived xenograft PDX) tumor model. (B): Certain data from another colorectal (CRC) patient derived xenograft PDX) tumor model.

FIG. 9. ¹H NMR of a preparation of I-66 as an example. Solvent: methanol-d4. About 10 mg/750 μL. Temperature: 298 K. Number of scans: 8. Receiver gain: 30.1. Relaxation Delay: 2.5000. Acquisition Time: 1.5139. Spectrometer Frequency: 900.30 MHz. Spectra Width: 10822.5. Lowest Frequency: −927.2. Acquired Size: 16384. Spectral Size: 65536. Digital Resolution: 0.17.

FIG. 10. A region of ¹H NMR of I-66 as an example. Top: 900 MHz ¹H of a preparation of I-66 in methanol-d4, 298 K, about 10 mg/750 μL. Bottom: 500 MHz ¹H of a preparation of I-66 in methanol-d4, 298 K, about 10 mg/500 μL. As shown here, the top spectra can provide improved resolution for various peaks.

FIG. 11. A region of ¹H NMR of I-66 as an example. Top: 900 MHz ¹H of a preparation of I-66 in methanol-d4, 298 K, about 10 mg/750 μL. Bottom: 500 MHz ¹H of a preparation of I-66 in methanol-d4, 298 K, about 10 mg/500 μL. As shown here, the top spectra can provide improved resolution for various peaks; certain NH peaks at about 7.7 ppm that were observed in the 500 MHz data were absent in the 900 MHz data. As those skilled in the art appreciate, NH peaks may change depending on sample environments.

FIG. 12. ¹³C NMR of a preparation of I-66 as an example. Solvent: methanol-d4. About 10 mg/750 μL. Temperature: 298 K. Number of scans: 8108. Receiver gain: 177.2. Relaxation Delay: 1.5000. Acquisition Time: 0.3277. Spectrometer Frequency: 226.40 MHz. Spectra Width: 50000.0. Lowest Frequency: −911.8. Acquired Size: 16384. Spectral Size: 65536. Digital Resolution: 0.76.

FIG. 13. COSY of a preparation of I-66 as an example. Solvent: methanol-d4. About 10 mg/750 μL. Temperature: 298 K. Number of scans: 4. Receiver gain: 30.1. Relaxation Delay: 2.0000. Acquisition Time: 0.2272. Spectrometer Frequency: (900.30, 900.30). Spectra Width: (9014.4, 9009.0). Lowest Frequency: (−22.7, −23.3). Nucleus: (¹H, ¹H). Acquired Size: (2048, 1024). Spectral Size: (2048, 1024). Digital Resolution: (4.40, 8.80).

FIG. 14. ¹H-¹³C HSQC of a preparation of I-66 as an example. Solvent: methanol-d4. About 10 mg/750 μL. Temperature: 298 K. Number of scans: 8. Receiver gain: 177.2. Relaxation Delay: 1.5000. Acquisition Time: 0.1137. Spectrometer Frequency: (900.30, 226.40). Spectra Width: (9009.0, 34013.6). Lowest Frequency: (−22.0, 288.5). Nucleus: (1H, 13C). Acquired Size: (1024, 512). Spectral Size: (1024, 1024). Digital Resolution: (8.80, 33.22).

FIG. 15. ¹H-¹³C HMBC of a preparation of I-66 as an example. Solvent: methanol-d4. About 10 mg/750 μL. Temperature: 298 K.

FIG. 16. ¹H-¹H TOCSY of a preparation of I-66 as an example. Solvent: methanol-d4. About 10 mg/750 μL. Temperature: 298 K. Number of scans: 8. Receiver gain: 57.7. Relaxation Delay: 2.0000. Acquisition Time: 0.2272. Spectrometer Frequency: (900.30, 900.30). Spectra Width: (9014.4, 9009.0). Lowest Frequency: (−21.8, −23.3). Acquired Size: (2048, 800). Spectral Size: (2048, 1024). Digital Resolution: (4.40, 8.80).

FIG. 17. ¹H-¹H NOESY of a preparation of I-66 as an example. Solvent: methanol-d4. About 10 mg/750 μL. Temperature: 298 K. Number of scans: 8. Receiver gain: 57.7. Relaxation Delay: 2.0000. Acquisition Time: 0.2272. Spectrometer Frequency: (900.30, 900.30). Spectra Width: (9014.4, 9009.0). Lowest Frequency: (−5.7, −3.0). Acquired Size: (2048, 1024). Spectral Size: (2048, 1024). Digital Resolution: (4.40, 8.80).

FIG. 18. Expansion of ¹H-¹H NOESY of a preparation of I-66 as an example. Solvent: methanol-d4. About 10 mg/750 μL. Temperature: 298 K. Number of scans: 8. Receiver gain: 57.7. Relaxation Delay: 2.0000. Acquisition Time: 0.2272. Spectrometer Frequency: (900.30, 900.30). Spectra Width: (9014.4, 9009.0). Lowest Frequency: (−5.7, −3.0). Acquired Size: (2048, 1024). Spectral Size: (2048, 1024). Digital Resolution: (4.40, 8.80). As shown herein, NOE peak between the olefin protons of the PL3-B5 staple (111 and 112) was observed. In some embodiments, this peak was stronger than the NOE peak between neighboring aromatic protons of 3Thi (20 and 21), and/or of the p and m protons in the phenyl group of Phe (48 and 47/49).

FIG. 19. Provided technologies can provide robust anti-tumor effects in vivo. Hepatocellular PDX models. Rectangles: vehicle (10 mg/mL arginine/6% PEG400 phosphate); filled circles: I-66. (A): beta-catenin D32V mutant. IP dosing Q4D, n=5/group. (B): beta-catenin D32H mutant. IP dosing Q7D, n=5/group.

FIG. 20. Provided technologies can provide robust anti-tumor effects in vivo. Hepatocellular PDX models. Tumor volume (mm³) assessed at multiple days after administration of various doses of I-66. Rectangles: vehicle (10 mM sodium phosphate dibasic, 10 mg/mL arginine and 6% PEG400 phosphate (pH 8.0)); filled circles: I-66. (A): beta-catenin D32V mutant. IP dosing Q4D, n=5/group. (B): beta-catenin D32H mutant. IP dosing Q7D, n=5/group. Error bars are standard error of the mean (SEM).

FIG. 21. Provided technologies can provide robust anti-tumor effects in vivo. Hepatocellular PDX models. Tumor volume (mm³) assessed at multiple days after administration of various doses of I-66. Rectangles: vehicle (10 mM sodium phosphate dibasic, 10 mg/mL arginine and 6% PEG400 phosphate (pH 8.0)); filled circles: I-66. (A): beta-catenin S45F mutant. IP dosing Q4D, n=5/group. (B): beta-catenin S37C mutant. IP dosing Q7D, n=5/group. Error bars are standard error of the mean (SEM).

### DETAILED DESCRIPTION OF CERTAIN EMBODIMENTS

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand (e.g., an agent) binds to its partner (e.g., beta-catenin or a portion thereof). Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound. In some embodiments, an agent is a stapled peptide.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Amino acid: In its broadest sense, as used herein, refers to any compound and/or substance that can be incorporated into a polypeptide chain, e.g., through formation of one or more peptide bonds. In some embodiments, an amino acid comprising an amino group and an a carboxylic acid group. In some embodiments, an amino acid has the structure of NH(R^{a1})-L^{a1}-C(R^{a2})(R^{a3})-L^{a2}-COOH, wherein each variable is independently as described in the present disclosure. In some embodiments, an amino acid has the general structure NH(R′)—C(R′)₂—COOH), wherein each R′ is independently as described in the present disclosure. In some embodiments, an amino acid has the general structure H₂N—C(R′)₂—COOH), wherein R′ is as described in the present disclosure. In some embodiments, an amino acid has the general structure H₂N—C(H)(R′)—COOH, wherein R′ is as described in the present disclosure. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, one or more hydrogens, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Aryl: The term “aryl” used alone or as part of a larger moiety as in “aralkyl,” “aralkoxy,” “aryloxyalkyl,” etc. refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. In some embodiments, also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like, where a radical or point of attachment is on an aryl ring.

Associated with: Two events or entities are “associated” with one another, as that term is used herein, if the presence, level and/or form of one is correlated with that of the other. For example, a particular entity (e.g., nucleic acid (e.g., genomic DNA, transcripts, mRNA, etc.), polypeptide, genetic signature, metabolite, microbe, etc.,) is considered to be associated with a particular disease, disorder, or condition, if its presence, level and/or form correlates with incidence of and/or susceptibility to the disease, disorder, or condition (e.g., across a relevant population).

Binding: It will be understood that the term “binding”, as used herein, typically refers to a non-covalent association between or among agents. In many embodiments herein, binding is addressed with respect to particular agents and beta-catenin. It will be appreciated by those of ordinary skill in the art that such binding may be assessed in any of a variety of contexts. In some embodiments, binding is assessed with respect to beta-catenin. In some embodiments, binding is assessed with respect to one or more amino acid residues of beta-catenin. In some embodiments, binding is assessed with respect to one or more amino acid residues corresponding to (e.g., similarly positioned in three dimensional space and/or having certain similar properties and/or functions) those of beta-catenin.

Binding site: The term “binding site”, as used herein, refers to a region of a target polypeptide, formed in three-dimensional space, that includes one or more or all interaction residues of the target polypeptide. In some embodiments, “binding site” may refer to one or more amino acid residues which comprise or are one or more or all interaction amino acid residues of a target polypeptide. As will be understood by those of ordinary skill in the art, a binding site may include residues that are adjacent to one another on a linear chain, and/or that are distal to one another on a linear chain but near to one another in three-dimensional space when a target polypeptide is folded. A binding site may comprise amino acid residues and/or saccharide residues.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic” may also include aliphatic rings that are fused to one or more aromatic or nonaromatic rings, such as decahydronaphthyl or tetrahydronaphthyl, where a radical or point of attachment is on an aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₁₀, or C₃-C₆ hydrocarbon, or a C₄-C₁₀, or C₈-C₁₀ bicyclic hydrocarbon that is completely saturated or that contains one or more units of unsaturation, but which is not aromatic, or a C₉-C₁₆ tricyclic hydrocarbon that is completely saturated or that contains one or more units of unsaturation, but which is not aromatic.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, in some embodiments, a peptide may be considered to be engineered if its amino acid sequence has been selected by man. For example, an engineered agent has an amino acid sequence that was selected based on preferences for corresponding amino acids at particular sites of protein-protein interactions. In some embodiments, an engineered sequence has an amino acid sequence that differs from the amino acid sequence of polypeptides included in the NCBI database that binds to a TCF site of beta-catenin. In many embodiments, provided agents are engineered agents. In some embodiments, engineered agents are peptide agents comprising non-natural amino acid residues, non-natural amino acid sequences, and/or peptide staples. In some embodiments, provided agents comprise or are engineered peptide agents which comprise engineered sequences.

Halogen: The term “halogen” means F, Cl, Br, or I.

Heteroaliphatic: The term “heteroaliphatic” is given its ordinary meaning in the art and refers to aliphatic groups as described herein in which one or more carbon atoms are replaced with one or more heteroatoms (e.g., oxygen, nitrogen, sulfur, silicon, phosphorus, and the like).

Heteroalkyl: The term “heteroalkyl” is given its ordinary meaning in the art and refers to alkyl groups as described herein in which one or more carbon atoms is replaced with a heteroatom (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-,” used alone or as part of a larger moiety, e.g., “heteroaralkyl,” or “heteroaralkoxy,” refer to monocyclic, bicyclic or polycyclic ring systems having, for example, a total of five to thirty, e.g., 5, 6, 9, 10, 14, etc., ring members, wherein at least one ring in the system is aromatic and at least one aromatic ring atom is a heteroatom. In some embodiments, a heteroatom is nitrogen, oxygen or sulfur. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where a radical or point of attachment is on a heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon and is not hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

Heterocyclyl: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where a radical or point of attachment is on a heteroaliphatic ring. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below (hydrophobicity scale of Kyte and Doolittle, 1982: A simple method for displaying the hydropathic character of a protein. J. Mol. Biol. 157:105-132):

| | | | | Side | Hydropathy |
|---|---|---|---|---|---|
| | | | Side | Chain | Index of |
| | 3 Letter | 1 Letter | Chain | Acidity/ | Kyte and |
| Amino Acid | Code | Code | Polarity | Basicity | Doolittle |
| Alanine | Ala | A | nonpolar | neutral | 1.8 |
| Arginine | Arg | R | polar | basic | −4.5 |
| Asparagine | Asn | N | polar | neutral | −3.5 |
| Aspartic acid | Asp | D | polar | acidic | −3.5 |
| Cysteine | Cys | C | nonpolar | neutral | 2.5 |
| Glutamic acid | Glu | E | polar | acidic | −3.5 |
| Glutamine | Gln | Q | polar | neutral | −3.5 |
| Glycine | Gly | G | nonpolar | neutral | −0.4 |
| Histidine | His | H | polar | basic | −3.2 |
| Isoleucine | Ile | I | nonpolar | neutral | 4.5 |
| Leucine | Leu | L | nonpolar | neutral | 3.8 |
| Lysine | Lys | K | polar | basic | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Interaction residues: The term “interaction residues”, “interaction motifs”, as used herein, refers to, with respect to an agent, residues or motifs in an agent that are designed to interact with particular target residues in a target polypeptide, or with respect to a target polypeptide, residues in a target polypeptide that interact with particular motifs (e.g., aromatic groups, amino acid residues, etc.) of an agent. Specifically, interaction residues and motifs of various agents are selected and arranged within the agents so that they will be displayed in three dimensional space within a predetermined distance (or volume) of identified target residues (e.g., upon binding, docking or other interaction assays). In many embodiments, interaction residues are direct-binding residues.

“Improved,” “increased”′ or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Peptide: The term “peptide” as used herein refers to a polypeptide. In some embodiments, a peptide is a polypeptide that is relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids. In some embodiments, a length is about 5-20, 5-19, 5-18, 5-17, 5-16, 5-15, 10-20, 10-19, 10-18, 10-17, 10-16, 10-15, 11-20, 11-19, 11-18, 11-17, 11-16, 11-15, 12-20, 12-19, 12-18, 12-17, 12-16, 12-15, 13-20, 13-19, 13-18, 13-17, 13-16, 13-15, 14-20, 14-19, 14-18, 14-17, 14-16, 14-15, or about 2, 3, 4, 5, 6, 7, 8, 9, 10, 11, 12, 13, 14, 15, 16, 17, 18, 19, or 20 amino acids.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; RingeR's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known. For example, S. M. Berge, et al. describes pharmaceutically acceptable salts in detail in J. Pharmaceutical Sciences, 66:1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other known methods such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds with bases. Representative alkali or alkaline earth metal salts include salts of sodium, lithium, potassium, calcium, magnesium, and the like. In some embodiments, pharmaceutically acceptable salts are ammonium salts (e.g., —N(R)₃⁺). In some embodiments, pharmaceutically acceptable salts are sodium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Protecting group: The term “protecting group,” as used herein, is well known in the art and includes those described in detail in Protecting Groups in Organic Synthesis, T. W. Greene and P. G. M. Wuts, 3rd edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromocthyl carbamate (DB-t-BOC), 1,1-dimethyl-2,2,2-trichlorocthyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino) acetamide, 3-(p-hydroxyphenyl) propanamide, 3-(0-nitrophenyl) propanamide, 2-methyl-2-(o-nitrophenoxy) propanamide, 2-methyl-2-(0-phenylazophenoxy) propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridonc, N-mcthylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten) carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In some embodiments, suitable mono-protected amines include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. In some embodiments, suitable di-protected amines include amines that are substituted with two substituents independently selected from those described above as mono-protected amines, and further include cyclic imides, such as phthalimide, maleimide, succinimide, and the like. In some embodiments, suitable di-protected amines include pyrroles and the like, 2,2,5,5-tetramethyl-[1,2,5]azadisilolidine and the like, and azide.

Suitably protected carboxylic acids further include, but are not limited to, silyl-, alkyl-, alkenyl-, aryl-, and arylalkyl-protected carboxylic acids. Examples of suitable silyl groups include trimethylsilyl, tricthylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2— and 4-picolyl. In some embodiments, suitable protected carboxylic acids include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Suitable hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy) methyl(p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinatc), 4,4-(cthylenedithio) pentanoate (levulinoyldithioacctal), pivaloate, adamantoate, crotonatc, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl) ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl) xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4—N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specificity: As is known in the art, “specificity” is a measure of the ability of a particular ligand (e.g., an agent) to distinguish its binding partner (e.g., beta-catenin) from other potential binding partners (e.g., another protein, another portion (e.g., domain) of beta-catenin).

Substitution: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In some embodiments, example substituents are described below.

Suitable monovalent substituents are halogen; —(CH₂)₀₋₄R^{○}; —(CH₂)₀₋₄OR^{○}; —O(CH₂)₀₋₄R^{○}, —O—(CH₂)₀₋₄C(O)OR^{○}; —(CH₂)₀₋₄CH(OR^{○})₂; —(CH₂)₀₋₄Ph, which may be substituted with R^{○}; —(CH₂)₀₋₄O(CH₂)₀₋₁Ph which may be substituted with R^{○}; —CH═CHPh, which may be substituted with R^{○}; —(CH₂)₀₋₄O(CH₂)_{0 1}-pyridyl which may be substituted with R^{○}; —NO₂; —CN; —N₃; —(CH₂)₀₋₄N(R^{○})₂; —(CH₂)₀₋₄N(R^{○}C(O)R^{○}; —N(R^{○}C(S)R^{○}; —(CH₂)₀₋₄N(R^{○}C(O)N(R^{○})₂; —N(R^{○}C(S)N(R^{○})₂; —(CH₂)₀₋₄N(R^{○}C(O)OR^{○}; —N(R^{○}N(R^{○}C(O)R^{○}; —N(R^{○}N(R^{○}C(O)N(R^{○})₂; —N(R^{○}N(R^{○}C(O)OR^{○}; —(CH₂)₀₋₄C(O)R^{○}; —C(S)R^{○}; —(CH₂)₀₋₄C(O)OR^{○}; —(CH₂)₀₋₄C(O)SR^{○}; —(CH₂)₀₋₄C(O)OSi(R^{○})₃; —(CH₂)₀₋₄OC(O)R^{○}; —OC(O)(CH₂)₀₋₄SR^{○}, —SC(S)SR^{○}; —(CH₂)₀₋₄SC(O)R^{○}; —(CH₂)₀₋₄C(O)N(R^{○})₂; —C(S)N(R^{○})₂; —C(S)SR^{○}; —SC(S)SR^{○}, —(CH₂)₀₋₄OC(O)N(R^{○})₂; —C(O)N(OR^{○})R^{○}; —C(O)C(O)R^{○}; —C(O)CH₂C(O)R^{○}; —C(NOR^{○})R^{○}; —(CH₂)₀₋₄SSR^{○}; —(CH₂)₀₋₄S(O)₂R^{○}; —(CH₂)₀₋₄S(O)₂OR^{○}; —(CH₂)₀₋₄OS(O)₂R^{○}; —S(O)₂N(R^{○})₂; —(CH₂)₀₋₄S(O)R^{○}; —N(R^{○})S(O)₂N(R^{○})₂; —N(R^{○})S(O)₂R^{○}; —N(OR^{○})R^{○}; —C(NH)N(R^{○})₂; —Si(R^{○})₃; —OSi(R^{○})₃; —P(R^{○})₂; —P(OR^{○})₂; —OP(R^{○})₂; —OP(OR^{○})₂; —N(R^{○})P(R^{○})₂; —B(R^{○})₂; —OB(R^{○})₂; —P(O)(R^{○})₂; —OP(O)(R^{○})₂; —N(R^{○})P(O)(R^{○})₂; —(C₁₋₄ straight or branched alkylene)O—N(R^{○})₂; or —(C₁₋₄ straight or branched alkylene)C(O)O—N(R^{○})₂; wherein each R^{○} may be substituted as defined below and is independently hydrogen, C₁₋₂₀ (e.g., C₁₋₁₅, C₁₋₁₀, C₁₋₆, C₁₋₄, etc.) aliphatic, C₁₋₂₀ (e.g., C₁₋₁₅, C₁₋₁₀, C₁₋₆, C₁₋₄, etc.) heteroaliphatic having 1-5 heteroatoms independently selected from nitrogen, oxygen, sulfur, silicon and phosphorus, —CH₂—(C₆₋₁₄ (e.g., C₆₋₁₀, C₆, C₁₀, etc.) aryl), —O(CH₂)₀₋₁(C₆₋₁₄ (e.g., C₆₋₁₀, C₆, C₁₀, etc.) aryl), —CH₂—(5-14 (e.g., 5-10, 5-9, 5-6, etc.) membered heteroaryl ring), a 3-20 (e.g., 3-15, 3-10, 3-6, 5-10, 5-6, 3, 4, 5, 6, 7, 8, 9, 10, 11, 12, 13, 14, 15, 16, 17, 18, 19, 20, etc.) membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{○}, taken together with their intervening atom(s), form a 3-20 (e.g., 3-15, 3-10, 3-6, 5-10, 5-6, 3, 4, 5, 6, 7, 8, 9, 10, 11, 12, 13, 14, 15, 16, 17, 18, 19, 20, etc.) membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{○} (or the ring formed by taking two independent occurrences of R^{○} together with their intervening atoms), are independently halogen, —(CH₂)₀₋₂R^{○},)-(haloR^{●}, (CH₂)₀₋₂OH, —(CH₂)₀₋₂OR^{○}, —(CH₂)₀₋₂CH(OR^{○})₂; —O(haloR^{●}), —CN, —N₃, —(CH₂)₀₋₂C(O)R^{○}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)_{0- 2}NR^{●}₂, —NO₂, —SiR^{●}₃, —OSiR^{●}₃, —C(O)SR^{●}, —(C_{1 4} straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3-6 (e.g., 3-5, 5-6, etc.)-membered saturated, partially unsaturated, or aryl ring having 0-4 heteroatoms independently selected from nitrogen, oxygen, and sulfur. Suitable divalent substituents on a saturated carbon atom of R^{○} include ═O and ═S.

Suitable divalent substituents are the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3-6 (e.g., 3-5, 5-6, etc.)-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include:—O(CR*₂)₂₋₃O—, wherein each independent occurrence of R* is selected from hydrogen, C₁₋₆ aliphatic which may be substituted as defined below, or an unsubstituted 3-6 (e.g., 3-5, 5-6, etc.)-membered saturated, partially unsaturated, or aryl ring having 0-4 heteroatoms independently selected from nitrogen, oxygen, and sulfur.

Suitable substituents on the aliphatic group of R* are halogen, —R^{●}, -(haloR^{●}), —OH, —OR^{●}, —O(haloR^{●}, —CN, —C(O)OH, —C(O)OR^{●}, —NH₂, —NHR^{●}, —NR^{●}₂, or —NO₂, wherein each R^{○} is unsubstituted or where preceded by “halo” is substituted only with one or more halogens, and is independently C₁₋₄ aliphatic, —CH₂Ph, —O(CH₂)₀₋₁Ph, or a 3-6 (e.g., 3-5, 5-6, etc.)-membered saturated, partially unsaturated, or aryl ring having 0-4 heteroatoms independently selected from nitrogen, oxygen, and sulfur.

In some embodiments, suitable substituents on a substitutable nitrogen are —R^{†}, —NR^{†}₂, —C(O)R^{†}, C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6 (e.g., 3-5, 5-6, etc.)-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12 (e.g., 3-10, 3-6, 5-10, 5-6, 3, 4, 5, 6, 7, 8, 9, 10, etc.) membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, —(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6 (e.g., 3-5, 5-6, etc.)-membered saturated, partially unsaturated, or aryl ring having 0-4 heteroatoms independently selected from nitrogen, oxygen, and sulfur.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, a subject is a human.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target polypeptide: A “target polypeptide”, as that term is used herein, is a polypeptide with which an agent interacts. In some embodiments, a target polypeptide is a beta-catenin polypeptide. In some embodiments, a target polypeptide comprises, consists essentially of, or is a binding site of beta-catenin polypeptide.

Target residue: A “target residue”, as that term is used herein, is a residue within a target polypeptide with which an agent is designed to interact. For example, an agent may be characterized by particular interaction motifs (e.g., aromatic groups as described herein) and/or residues (e.g., amino acid residues comprising aromatic groups as described herein) selected and arranged (by virtue of being presented on the selected scaffold) to be within a certain predetermined distance (or volume) of a target residue. In some embodiments, a target residue is or comprises an amino acid residue.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated” as used herein, means that a moiety has one or more units of unsaturation.

Unless otherwise specified, salts, such as pharmaceutically acceptable acid or base addition salts, stereoisomeric forms, and tautomeric forms, of provided compound are included.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

### Stapled Peptides

In some embodiments, a provided agent is or comprises a peptide. In some embodiments, a provided agent is a peptide. In some embodiments, a peptide is a stapled peptide. In some embodiments, a provided agent is a stapled peptide. In some embodiments, a peptide is a stitched peptide. In some embodiments, a provided agent is a stitched peptide. In some embodiments, a stitched peptide comprises two or more staples, wherein two staples are bonded to the same peptide backbone atom. Stapled peptides as described herein are typically peptides in which two or more amino acids of a peptide chain are linked through connection of two peptide backbone atoms of the amino acid residues and, as is understood by those skilled in the art, the connection is not through the peptide backbone between the linked amino acid residues. In some embodiments, a staple as described herein is a linker that link one amino acid residue to another amino acid residue, e.g., through bonding to a peptide backbone atom of each of the amino acid residues and, as is understood by those skilled in the art, the connection through a staple is not through the peptide backbone between the linked amino acid residues. In some embodiments, a staple bonds to the peptide backbone by replacing one or more hydrogen and/or substituents (e.g., side chains, O, S, etc.) on peptide backbone atoms (e.g., C, N, etc.). In some embodiments, side chains form portions of staples. In some embodiments, a staple is bonded to two carbon backbone atoms, e.g., two alpha carbon atoms. In some embodiments, a staple comprises C(R′)₂ or N(R′), either individually or as part of a large moiety, wherein R′ is R and is taken together with another group attached to a backbone atom which can be R (e.g., R^{a3}) and their intervening atoms to form a ring as described herein (e.g., when PyrS2 is stapled in various peptides).

In some embodiments, a stapled peptide comprises one or more staples. In some embodiments, a stapled peptide comprises two or more staples. In some embodiments, a stapled peptide comprises three or more staples. In some embodiments, a stapled peptide comprises four or more staples. In some embodiments, there are three staples in a stapled peptide. In some embodiments, there are four staples in a stapled peptide.

As will be appreciated by those of ordinary skill in the art, a variety of peptide stapling technologies are available, including both hydrocarbon-stapling and non-hydrocarbon-stapling technologies, and can be utilized in accordance with the present disclosure. Various technologies for stapled and stitched peptides, including various staples and/or methods for manufacturing are available and may be utilized in accordance with the present disclosure, e.g., those described in WO 2019/051327, WO 2020/041270, WO 2022/020652, WO 2022/261257, etc., the staples of each of which are incorporated herein by reference.

In some embodiments, a peptide, e.g., a stapled peptide, is or comprise a helical structure. In some embodiments, a peptide is a stapled peptide. In some embodiments, a stapled peptide is as described in WO 2022/261257 which is incorporated herein by reference. In some embodiments, an peptide is as described in WO 2022/261257. In some embodiments, an agent is as described in WO 2022/261257.

In some embodiments, a staple is a hydrocarbon staple. In some embodiments, a staple as described herein is a non-hydrocarbon staple. In some embodiments, a non-hydrocarbon staple comprises one or more chain heteroatoms wherein a chain of a staple is the shortest covalent connection within the staple from one end of the staple to the other end of the staple.

In some embodiments, a staple comprises one or more amino groups, e.g., —N(R′)—, wherein each R′ is independently as described herein. In some embodiments, —N(R′)— bonds to two carbon atoms. In some embodiments, —N(R′)— bonds to two carbon atoms, wherein neither of the two carbon atoms are bond to any heteroatoms through a double bond. In some embodiments, —N(R′)— bonds to two sp3 carbon atoms. In some embodiments, a staple comprises one or more —C(O)—N(R′)— groups, wherein each R′ is independently as described herein. In some embodiments, a staple comprises one or more carbamate groups, e.g., one or more —(O)—C(O)—N(R′)—, wherein each R′ is independently as described herein. In some embodiments, R′ is —H. In some embodiments, R′ is optionally substituted C₁₋₆ aliphatic. In some embodiments, R′ is optionally substituted C₁₋₆ alkyl. In some embodiments, R′ is C₁₋₆ aliphatic. In some embodiments, R′ is C₁₋₆ alkyl. In some embodiments, R′ is methyl.

In some embodiments, peptides, e.g., staple peptides, of the present disclosure is or comprises a helix structure. As those skilled in the art will appreciate, helixes can have various lengths. In some embodiments, lengths of helixes range from 5 to 30 amino acid residues. In some embodiments, a length of a helix is 5, 6, 7, 8, 9, 10, 11, 12, 13, 14, 15, 16, 17, 18, 19, or 20, or more, amino acid residues.

Amino acids stapled together can have various number of amino acid residues in between, e.g., 1-20, 1-15, 1-10, 1, 2, 3, 4, 5, 6, 7, 8, 9, or 10, etc. In some embodiments, a staple is (i, i+4) which means there are three amino acid residues between the two amino acids (at positions i and i+4, respectively) that bond to the staple (at positions i+1, i+2, i+3, respectively). In some embodiments, a staple is (i, i+2). In some embodiments, a staple is (i, i+3). In some embodiments, a staple is (i, i+5). In some embodiments, a staple is (i, i+6). In some embodiments, a staple is (i, i+7). In some embodiments, a staple is (i, i+8). In some embodiments, a stapled peptide comprises two staples, one is (i, i+2) and the other is (i, i+7). In some embodiments, a stapled peptide comprises two staples, one is (i, i+3) and the other is (i, i+7). In some embodiments, a stapled peptide comprises two staples, one is (i, i+3) and the other is (i, i+4). In some embodiments, a stapled peptide comprises two staples, one is (i, i+4) and the other is (i, i+7). In some embodiments, a stapled peptide comprises two staples, one is (i, i+3) and the other is (i, i+3). In some embodiments, a stapled peptide comprises two staples, one is (i, i+4) and the other is (i, i+4). In some embodiments, a stapled peptide comprises two staples, one is (i, i+7) and the other is (i, i+7). In some embodiments, the two staples are bonded to a common backbone atom, e.g., an alpha carbon atom of an amino acid residue. In some embodiments, a stapled peptide further comprises a third staple. In some embodiments, a third staple is (i, i+3). In some embodiments, a third staple is (i, i+4). In some embodiments, a third staple is (i, i+7). In some embodiments, a stapled peptide further comprises a fourth staple. In some embodiments, a fourth staple is (i, i+3). In some embodiments, a fourth staple is (i, i+4). In some embodiments, a fourth staple is (i, i+7).

In some embodiments, a staple can be of various lengths, in some embodiments, as represent by the number of chain atoms of a staple. In some embodiments, a chain of a staple is the shortest covalent connection in the staple from a first end (connection point with a peptide backbone) of a staple to a second end of the staple, wherein the first end and the second end are connected to two different peptide backbone atoms. In some embodiments, a staple comprises 5-30 chain atoms, e.g., 5-20, 5-15, 5, 6, 7, 8, 9, or 10 to 11, 12, 13, 14, 15, 16, 17, 18, 19, 20, 21, 22, 23, 24, or 25 chain atoms. In some embodiments, a staple comprises 5 chain atoms. In some embodiments, a staple comprises 6 chain atoms. In some embodiments, a staple comprises 7 chain atoms. In some embodiments, a staple comprises 8 chain atoms. In some embodiments, a staple comprises 9 chain atoms. In some embodiments, a staple comprises 10 chain atoms. In some embodiments, a staple comprises 11 chain atoms. In some embodiments, a staple comprises 12 chain atoms. In some embodiments, a staple comprises 13 chain atoms. In some embodiments, a staple comprises 14 chain atoms. In some embodiments, a staple comprises 15 chain atoms. In some embodiments, a staple comprises 16 chain atoms. In some embodiments, a staple comprises 17 chain atoms. In some embodiments, a staple comprises 18 chain atoms. In some embodiments, a staple comprises 19 chain atoms. In some embodiments, a staple comprises 20 chain atoms. In some embodiments, a staple has a length of 5 chain atoms. In some embodiments, a staple has a length of 6 chain atoms. In some embodiments, a staple has a length of 7 chain atoms. In some embodiments, a staple has a length of 8 chain atoms. In some embodiments, a staple has a length of 9 chain atoms. In some embodiments, a staple has a length of 10 chain atoms. In some embodiments, a staple has a length of 11 chain atoms. In some embodiments, a staple has a length of 12 chain atoms. In some embodiments, a staple has a length of 13 chain atoms. In some embodiments, a staple has a length of 14 chain atoms. In some embodiments, a staple has a length of 15 chain atoms. In some embodiments, a staple has a length of 16 chain atoms. In some embodiments, a staple has a length of 17 chain atoms. In some embodiments, a staple has a length of 18 chain atoms. In some embodiments, a staple has a length of 19 chain atoms. In some embodiments, a staple has a length of 20 chain atoms. In some embodiments, a staple has a length of 8-15 chain atoms. In some embodiments, a staple has 8-12 chain atoms. In some embodiments, a staple has 9-12 chain atoms. In some embodiments, a staple has 9-10 chain atoms. In some embodiments, a staple has 8-10 chain atoms. In some embodiments, length of a staple can be adjusted according to the distance of the amino acid residues it connects, for example, a longer staple may be utilized for a (i, i+7) staple than a (i, i+4) or (i, i+3) staple. In some embodiments, a (i, i+2) staple has about 5-10, 5-8, e.g., about 5, 6, 7, 8, 9 or 10 chain atoms. In some embodiments, a (i, i+2) staple has 5 chain atoms. In some embodiments, a (i, i+2) staple has 6 chain atoms. In some embodiments, a (i, i+2) staple has 7 chain atoms. In some embodiments, a (i, i+2) staple has 8 chain atoms. In some embodiments, a (i, i+2) staple has 9 chain atoms. In some embodiments, a (i, i+2) staple has 10 chain atoms. In some embodiments, a (i, i+3) staple has about 5-10, 5-8, e.g., about 5, 6, 7, 8, 9 or 10 chain atoms. In some embodiments, a (i, i+3) staple has 5 chain atoms. In some embodiments, a (i, i+3) staple has 6 chain atoms. In some embodiments, a (i, i+3) staple has 7 chain atoms. In some embodiments, a (i, i+3) staple has 8 chain atoms. In some embodiments, a (i, i+3) staple has 9 chain atoms. In some embodiments, a (i, i+3) staple has 10 chain atoms. In some embodiments, a (i, i+4) staple has about 5-12, 5-10, 7-12, 5-8, e.g., about 5, 6, 7, 8, 9, 10, 11 or 12 chain atoms. In some embodiments, a (i, i+4) staple has 5 chain atoms. In some embodiments, a (i, i+4) staple has 6 chain atoms. In some embodiments, a (i, i+4) staple has 7 chain atoms. In some embodiments, a (i, i+4) staple has 8 chain atoms. In some embodiments, a (i, i+4) staple has 9 chain atoms. In some embodiments, a (i, i+4) staple has 10 chain atoms. In some embodiments, a (i, i+4) staple has 11 chain atoms. In some embodiments, a (i, i+4) staple has 12 chain atoms. In some embodiments, a (i, i+7) staple has about 8-25, 10-25, 10-16, 12-15, e.g., about 8, 9, 10, 11, 12, 13, 14, 15, 16, 17, 18, 19, 20, 21, 22, 23, 24 or 25 chain atoms. In some embodiments, a (i, i+7) staple has 8 chain atoms. In some embodiments, a (i, i+7) staple has 9 chain atoms. In some embodiments, a (i, i+7) staple has 10 chain atoms. In some embodiments, a (i, i+7) staple has 11 chain atoms. In some embodiments, a (i, i+7) staple has 12 chain atoms. In some embodiments, a (i, i+7) staple has 13 chain atoms. In some embodiments, a (i, i+7) staple has 14 chain atoms. In some embodiments, a (i, i+7) staple has 15 chain atoms. In some embodiments, a (i, i+7) staple has 16 chain atoms. In some embodiments, a (i, i+7) staple has 17 chain atoms. In some embodiments, a (i, i+7) staple has 18 chain atoms. In some embodiments, a (i, i+7) staple has 19 chain atoms. In some embodiments, a (i, i+7) staple has 20 chain atoms. In some embodiments, a (i, i+7) staple has 21 chain atoms. In some embodiments, a (i, i+7) staple has 22 chain atoms. In some embodiments, a stapled peptide comprises three or more staples, each of which is independently such a (I, i+2), (i, i+3), (i, i+4) or (i, i+7) staple. In some embodiments, a stapled peptide comprises such a (i, i+2) staple, such a (i, i+4) staple and such a (i, i+7) staple. In some embodiments, a stapled peptide comprises such a (i, i+3) staple, such a (i, i+4) staple and such a (i, i+7) staple. In some embodiments, a stapled peptide comprises such a (i, i+3) staple, such a (i, i+7) staple and such a (i, i+7) staple.

Staple lengths may be otherwise described. For example, in some embodiments, staple lengths may be described as the total number of chain atoms and non-chain ring atoms, where a non-chain ring atom is an atom of the staple which forms a ring with one or more chain atoms but is not a chain atom in that it is not within the shortest covalent connection from a first end of the staple to a second end of the staple. In some embodiments, staples formed using Monomer A (which comprises an azetidine moiety), Monomer B (which comprises a pyrrolidine moiety), and/or Monomer C (which comprises a pyrrolidine moiety), etc., may comprise one or two non-chain ring atoms.

In some embodiments, a staple has no heteroatoms in its chain. In some embodiments, a staple comprises at least one heteroatom in its chain. In some embodiments, a staple comprises at least one nitrogen atom in its chain.

In some embodiments, a staple is L^{s}, wherein L^{s} is an optionally substituted, bivalent C₈₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₉₋₁₃ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₀₋₁₅ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₁₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is a (i, i+2) staple in that not including the two amino acid residues that are directly connected to the staple, there are one amino acid residue between the two amino acid residues that are directly connected to the staple. In some embodiments, a staple is a (i, i+3) staple in that not including the two amino acid residues that are directly connected to the staple, there are two amino acid residues between the two amino acid residues that are directly connected to the staple. In some embodiments, a staple is a (i, i+4) staple in that not including the two amino acid residues that are directly connected to the staple, there are three amino acid residues between the two amino acid residues that are directly connected to the staple. In some embodiments, a staple is a (i, i+7) staple in that not including the two amino acid residues that are directly connected to the staple, there are six amino acid residues between the two amino acid residues that are directly connected to the staple.

In some embodiments, a staple comprises a double bond. In some embodiments, a staple comprises a double bond may be formed by olefin metathesis of two olefins. In some embodiments, staples are formed by metathesis reactions, e.g., involving one or more double bonds in amino acid residues as described herein. In some embodiments, a first amino acid residue comprising an olefin (e.g., AA1—CH═CH₂) and a second amino acid residue comprising an olefin (e.g., AA2—CH═CH₂) are stapled (e.g., forming AA1—CH═CH-AA2, wherein AA1 and AA2 are typically linked through one or more amino acid residues). In some embodiments, an olefin, e.g., in a staple, is converted into —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—. In some embodiments, each of the two olefins is independently of a side chain of an amino acid residue. In some embodiments, each olefin is independently a terminal olefin. In some embodiments, each olefin is independently a mono-substituted olefin.

In some embodiments, amino acids that are useful for stapling, or are stapled, are described in WO 2022/261257, e.g., those of formula A-II, A-II-b, A-III, A-IV, A-V, or A-VI, or salts thereof, which are incorporated herein by reference.

In some embodiments, an amino acid is selected from Tables A-I, A-II, A-III and A-IV (may be presented as Fmoc-protected). As appreciated by those skilled in the art, among other things, when incorporated into peptides, Fmoc-protected amino groups and carboxyl groups may independently form amide connections with other amino acid residues (or N- or C-terminus capping groups, or exist as N- or C-terminus amino or carboxyl groups). Olefins, including those in Alloc groups, may be utilized to form staples through olefin metathesis. Staples comprising olefins may be further modified, e.g., through hydrogenation to convert olefin double bonds into single bonds, and/or through CO₂ extrusion to convert carbamate moieties (e.g., —O—(CO)—N(R′)—) into amine moieties (e.g., —N(R′)—). In some embodiments, an agent is or comprises a stapled peptide (e.g., a stapled peptide described according to Table E2 or Table E3) or a salt thereof, in which stapled peptide each double bond is converted into a single bond. In some embodiments, a conversion is achieved through hydrogenation which adds a-H to each olefin carbon atom. In some embodiments, an olefin double bond is replaced with —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—.

TABLE A-I
Exemplary amino acids (Fmoc-Protected).
Monomer A (M_{A})

Monomer B (M_{B})

Monomer C (M_{C})

TABLE A-II
Exemplary amino acids (Fmoc-Protected).
Monomer D (M_{D})

Monomer E (M_{E})

Monomer F (M_{F})

Monomer G (M_{G})

Monomer H (M_{H})

Monomer I (M_{I})

TABLE A-III
Exemplary amino acids (Fmoc-Protected).
S3

R3

S4

R4

S5

R5

B5

S6

R6

S7

R7

S8

R8

n is 1-5, e.g., 1, 2 or 3

n is 1-5, e.g., 1, 2 or 3
PL3

PyrS

PyrS1

PyrS2

PyrS3

RdN

RcN

RgN

S₁₀

SdN

ScN

SgN

In some embodiments, an amino acid is an alpha-amino acid. In some embodiments, an amino acid is an L-amino acid. In some embodiments, an amino acid is a D-amino acid. In some embodiments, the alpha-carbon of an amino acid is achiral. In some embodiments, an amino acid is a beta-amino acid. In some embodiments, an amino acid is a gamma-amino acid.

In some embodiments, an olefin in a staple is a Z-olefin. In some embodiments, an olefin in a staple in an E-olefin. In some embodiments, a provided composition comprises stapled peptides comprising a staple that contains a Z-olefin and stapled peptides comprising a staple that contains an E-olefin. In some embodiments, a provided composition comprises stapled peptides comprising a staple that contains a Z-olefin. In some embodiments, a provided composition comprises stapled peptides comprising a staple that contains an E-olefin. In some embodiments, otherwise identical stapled peptides that differ only in the E/Z configuration of staple olefin demonstrate different properties and/or activities as demonstrated herein. In some embodiments, stapled peptides with E-olefin in a staple may provide certain desirable properties and/or activities given the context. In some embodiments, stapled peptides with Z-olefin in a staple may provide certain desirable properties and/or activities given the context.

In some embodiments, the present disclosure provides compositions comprising stapled peptides. In some embodiments, a composition comprises one and only one stereoisomer of a stapled peptide (e.g., E or Z isomer, and/or a single diastereomer/enantiomer with respect to a chiral center, etc.). In some embodiments, a composition comprises two or more stereoisomers (e.g., both E and Z isomers of one or more double bonds, and/or one or more diastereomers/enantiomers with respect to a chiral center, etc.). In some embodiments, a composition corresponds to a single peak in a chromatographic separation, e.g., HPLC. In some embodiments, a peak comprises one and only one stereoisomers. In some embodiments, a peak comprises two or more stereoisomers.

In some embodiments, two staples may be bonded to the same atom of the peptide backbone, forming a stitched peptide. In some embodiments, a staple is pro-lock wherein one end of the staple is bonded to the alpha-carbon of a proline residue. In some embodiments, a staple is a staple illustrated in Tables S-1, S-2, S-3, S-4, S-5 or S-6 of WO 2022/261257.

In some embodiments, the double bond in a (i, i+3) staple is Z. In some embodiments, the double bond in a (i, i+4) staple is Z. In some embodiments, the double bond in a (i, i+7) staple is Z. In some embodiments, the olefin is E. In some embodiments, the double bond in a (i, i+3) staple is E. In some embodiments, the double bond in a (i, i+4) staple is E. In some embodiments, the double bond in a (i, i+7) staple is E.

In some embodiments, a staple comprises —S—. In some embodiments, a staple is formed from cysteine stapling. Certain such staples are described in, e.g., WO 2022/261257.

Certain useful agents (peptides prior to stapling and stapled peptides post stapling) and compositions thereof are presented in Table E2 or Table E3 as examples, which includes various amino acid residues and N- and C-terminus capping groups for various positions as examples; also illustrated are various stapling patterns, e.g., X¹—X⁴—X¹¹, X¹—X³, X³—X⁷, X³—X¹⁰, X⁴-X¹¹, X⁷-X¹⁰, X⁷-X¹⁴, X¹⁰-X¹⁴, etc. As demonstrated herein, provided technologies can deliver improved useful properties and/or activities.

In some embodiments, a provided agent, a peptide, or a stapled peptide is a compound as described herein. In some embodiments, a provided agent has a structure selected from Table E2 or Table E3, or a salt thereof. In some embodiments, a provided agent is a stereoisomer of a structure selected from Table E2 or Table E3, or a salt thereof. In some embodiments, a provided agent is a stereoisomer, with respect to a chiral center bonded to two staples (e.g., in B4, B5, etc.), of a structure selected from Table E2 or Table E3, or a salt thereof. In some embodiments, a provided agent is a stereoisomer, with respect to olefin double bond(s) in staple(s), of a structure selected from Table E2 or Table E3, or a salt thereof. In some embodiments, a provided agent is a stereoisomer, with respect to olefin double bond(s) in staple(s) and/or a chiral center bonded to two staples (e.g., in B4, B5, etc.), of a structure selected from Table E2 or Table E3, or a salt thereof. In some embodiments, a provided composition is a composition described in Table E2 or Table E3. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a compound has the structure of

or a salt thereof. In some embodiments, a double bond of a (i, i+2) staple is E. In some embodiments, a double bond of a (i, i+2) staple is Z. In some embodiments, a double bond of a (i, i+3) staple is E. In some embodiments, a double bond of a (i, i+3) staple is Z. In some embodiments, a double bond of a (i, i+7) staple is E. In some embodiments, a double bond of a (i, i+7) staple is Z. In some embodiments, both double bonds are E. In some embodiments, both double bonds are Z. In some embodiments, a (i, i+3) staple is E, and the other is Z. In some embodiments, a (i, i+3) staple is Z, and the other is E. In some embodiments, a (i, i+4) staple is E, and the other is Z. In some embodiments, a (i, i+4) staple is Z, and the other is E. In some embodiments, a double bond of a (i, i+7) staple is Z, and a double bond of a second staple (e.g., (i, i+2), (i, i+3), (i, i+4), etc.) is E. In some embodiments, a double bond of a (i, i+7) staple is Z, and a double bond of a second staple (e.g., (i, i+2), (i, i+3), (i, i+4), etc.) is Z. In some embodiments, a double bond of a (i, i+7) staple is E, and a double bond of a second staple (e.g., (i, i+2), (i, i+3), (i, i+4), etc.) is E. In some embodiments, a double bond of a (i, i+7) staple is E, and a double bond of a second staple (e.g., (i, i+2), (i, i+3), (i, i+4), etc.) is Z. In some embodiments, two staples are bonded to a chiral center (e.g., a carbon atom in B5), and the chiral center is R. In some embodiments, two staples are bonded to a chiral center (e.g., a carbon atom in B5), and the chiral center is S.

In some embodiments, a compound has the structure selected from below or a salt thereof:

In some embodiments, an agent is SP-1-1 or a salt thereof. In some embodiments, an agent is SP-1-2 or a salt thereof. In some embodiments, an agent is SP-1-3 or a salt thereof. In some embodiments, an agent is SP-1-4 or a salt thereof. In some embodiments, an agent is SP-1-5 or a salt thereof. In some embodiments, an agent is SP-1-6 or a salt thereof. In some embodiments, an agent is SP-1-7 or a salt thereof. In some embodiments, an agent is SP-1-8 or a salt thereof. In some embodiments, an agent is SP-2-1 or a salt thereof. In some embodiments, an agent is SP-2-2 or a salt thereof. In some embodiments, an agent is SP-2-3 or a salt thereof. In some embodiments, an agent is SP-2-4 or a salt thereof. In some embodiments, an agent is SP-2-5 or a salt thereof. In some embodiments, an agent is SP-2-6 or a salt thereof. In some embodiments, an agent is SP-2-7 or a salt thereof. In some embodiments, an agent is SP-2-8 or a salt thereof. In some embodiments, an agent is SP-3-1 or a salt thereof. In some embodiments, an agent is SP-3-2 or a salt thereof. In some embodiments, an agent is SP-4-1 or a salt thereof. In some embodiments, an agent is SP-4-2 or a salt thereof. In some embodiments, an agent is SP-4-3 or a salt thereof. In some embodiments, an agent is SP-4-4 or a salt thereof. In some embodiments, an agent is SP-4-5 or a salt thereof. In some embodiments, an agent is SP-4-6 or a salt thereof. In some embodiments, an agent is SP-4-7 or a salt thereof. In some embodiments, an agent is SP-4-8 or a salt thereof. In some embodiments, an agent is SP-5-1 or a salt thereof. In some embodiments, an agent is SP-5-2 or a salt thereof. In some embodiments, an agent is SP-5-3 or a salt thereof. In some embodiments, an agent is SP-5-4 or a salt thereof. In some embodiments, an agent is SP-5-5 or a salt thereof. In some embodiments, an agent is SP-5-6 or a salt thereof. In some embodiments, an agent is SP-5-7 or a salt thereof. In some embodiments, an agent is SP-5-8 or a salt thereof. In some embodiments, an agent is SP-6 or a salt thereof. In some embodiments, an agent is SP-7-1 or a salt thereof. In some embodiments, an agent is SP-7-2 or a salt thereof. In some embodiments, an agent is SP-7-3 or a salt thereof. In some embodiments, an agent is SP-7-4 or a salt thereof. In some embodiments, an agent is SP-7-5 or a salt thereof. In some embodiments, an agent is SP-7-6 or a salt thereof. In some embodiments, an agent is SP-7-7 or a salt thereof. In some embodiments, an agent is SP-7-8 or a salt thereof. In some embodiments, an agent is SP-8-1 or a salt thereof. In some embodiments, an agent is SP-8-2 or a salt thereof. In some embodiments, an agent is SP-8-3 or a salt thereof. In some embodiments, an agent is SP-8-4 or a salt thereof. In some embodiments, an agent is SP-8-5 or a salt thereof. In some embodiments, an agent is SP-8-6 or a salt thereof. In some embodiments, an agent is SP-8-7 or a salt thereof. In some embodiments, an agent is SP-8-8 or a salt thereof. In some embodiments, an agent is SP-9-1 or a salt thereof. In some embodiments, an agent is SP-9-2 or a salt thereof. In some embodiments, an agent is SP-9-3 or a salt thereof. In some embodiments, an agent is SP-9-4 or a salt thereof. In some embodiments, an agent is SP-9-5 or a salt thereof. In some embodiments, an agent is SP-9-6 or a salt thereof. In some embodiments, an agent is SP-9-7 or a salt thereof. In some embodiments, an agent is SP-9-8 or a salt thereof. In some embodiments, an agent is SP-10-1 or a salt thereof. In some embodiments, an agent is SP-10-2 or a salt thereof. In some embodiments, an agent is SP-10-3 or a salt thereof. In some embodiments, an agent is SP-10-4 or a salt thereof. In some embodiments, an agent is SP-10-5 or a salt thereof. In some embodiments, an agent is SP-10-6 or a salt thereof. In some embodiments, an agent is SP-10-7 or a salt thereof. In some embodiments, an agent is SP-10-8 or a salt thereof. In some embodiments, an agent is SP-11-1 or a salt thereof. In some embodiments, an agent is SP-11-2 or a salt thereof. In some embodiments, an agent is SP-11-3 or a salt thereof. In some embodiments, an agent is SP-11-4 or a salt thereof. In some embodiments, an agent is SP-11-5 or a salt thereof. In some embodiments, an agent is SP-11-6 or a salt thereof. In some embodiments, an agent is SP-11-7 or a salt thereof. In some embodiments, an agent is SP-11-8 or a salt thereof. In some embodiments, an agent is SP-12-1 or a salt thereof. In some embodiments, an agent is SP-12-2 or a salt thereof. In some embodiments, an agent is SP-12-3 or a salt thereof. In some embodiments, an agent is SP-12-4 or a salt thereof. In some embodiments, an agent is SP-12-5 or a salt thereof. In some embodiments, an agent is SP-12-6 or a salt thereof. In some embodiments, an agent is SP-12-7 or a salt thereof. In some embodiments, an agent is SP-12-8 or a salt thereof. In some embodiments, an agent is SP-13-1 or a salt thereof. In some embodiments, an agent is SP-13-2 or a salt thereof. In some embodiments, an agent is SP-13-3 or a salt thereof. In some embodiments, an agent is SP-13-4 or a salt thereof. In some embodiments, an agent is SP-13-5 or a salt thereof. In some embodiments, an agent is SP-13-6 or a salt thereof. In some embodiments, an agent is SP-13-7 or a salt thereof. In some embodiments, an agent is SP-13-8 or a salt thereof. In some embodiments, an agent is SP-14-1 or a salt thereof. In some embodiments, an agent is SP-14-2 or a salt thereof. In some embodiments, an agent is SP-14-3 or a salt thereof. In some embodiments, an agent is SP-14-4 or a salt thereof. In some embodiments, an agent is SP-14-5 or a salt thereof. In some embodiments, an agent is SP-14-6 or a salt thereof. In some embodiments, an agent is SP-14-7 or a salt thereof. In some embodiments, an agent is SP-14-8 or a salt thereof. In some embodiments, an agent is SP-15-1 or a salt thereof. In some embodiments, an agent is SP-15-2 or a salt thereof. In some embodiments, an agent is SP-15-3 or a salt thereof. In some embodiments, an agent is SP-15-4 or a salt thereof. In some embodiments, an agent is SP-15-5 or a salt thereof. In some embodiments, an agent is SP-15-6 or a salt thereof. In some embodiments, an agent is SP-15-7 or a salt thereof. In some embodiments, an agent is SP-15-8 or a salt thereof.

In some embodiments, an agent is selected from:

or a salt thereof.

Agents, e.g., peptides including stapled peptides, can contain various numbers of amino acid residues. In some embodiments, a length of a peptide agent is about 5-20, 5-19, 5-18, 5-17, 5-16, 5-15, 10-20, 10-19, 10-18, 10-17, 10-16, 10-15, 11-20, 11-19, 11-18, 11-17, 11-16, 11-15, 12-20, 12-19, 12-18, 12-17, 12-16, 12-15, 13-20, 13-19, 13-18, 13-17, 13-16, 13-15, 14-20, 14-19, 14-18, 14-17, 14-16, 14-15, or about 2, 3, 4, 5, 6, 7, 8, 9, 10, 11, 12, 13, 14, 15, 16, 17, 18, 19, or 20 amino acid residues. In some embodiments, a length is about 10 amino acid residues. In some embodiments, a length is about 11 amino acid residues. In some embodiments, a length is about 12 amino acid residues. In some embodiments, a length is about 13 amino acid residues. In some embodiments, a length is about 14 amino acid residues. In some embodiments, a length is about 15 amino acid residues. In some embodiments, a length is about 16 amino acid residues. In some embodiments, a length is about 17 amino acid residues. In some embodiments, a length is about 18 amino acid residues. In some embodiments, a length is about 19 amino acid residues. In some embodiments, a length is about 20 amino acid residues.

In some embodiments, as described herein, one or more staples independently comprise an olefin double bond (e.g., formed through olefin metathesis). In some embodiments, one or more staples independently comprise an amide group (e.g., formed through amidation). In some embodiments, at least one staple does not contain an olefin double bond. In some embodiments, there is at least one staple whose formation does not comprise reactions of olefins such as olefin metathesis and/or modification of olefin double bonds (e.g., hydrogenation, epoxidation, etc.).

In some embodiments, a residue of a staple (e.g., B5) is so positioned that if its position is P (e.g., X⁴), a first acidic amino acid residue is at position P-2 (e.g., X²), a second acidic amino acid residue is positioned at P+1 (e.g., X⁵), a third acidic amino acid residue is positioned at P+2 (e.g., X⁶), a hydrophobic amino acid residue is positioned at P+4 (e.g., X⁸), a first aromatic amino acid residue is positioned at P+5 (e.g., X⁹), a second aromatic amino acid residue is positioned at P+8 (e.g., X¹²), and/or a third aromatic amino acid residue is positioned at P+9 (e.g., X¹³). In some embodiments, a staple is a (i, i+7) staple, and the other residue of the staple is positioned at P+7 (e.g., X¹¹). In some embodiments, a first acidic amino acid residue is at position P-2 (e.g., X²). In some embodiments, a second acidic amino acid residue is positioned at P+1 (e.g., X⁵). In some embodiments, a third acidic amino acid residue is positioned at P+2 (e.g., X⁶). In some embodiments, a hydrophobic amino acid residue is positioned at P+4 (e.g., X⁸). In some embodiments, a first aromatic amino acid residue is positioned at P+5 (e.g., X⁹). In some embodiments, a second aromatic amino acid residue is positioned at P+8 (e.g., X¹²). In some embodiments, a third aromatic amino acid residue is positioned at P+9 (e.g., X¹³). In some embodiments, a first acidic amino acid residue is at position P-2 (e.g., X²), a second acidic amino acid residue is positioned at P+1 (e.g., X⁵), a first aromatic amino acid residue is positioned at P+5 (e.g., X⁹), a second aromatic amino acid residue is positioned at P+8 (e.g., X¹²), and a third aromatic amino acid residue is positioned at P+9 (e.g., X¹³). In some embodiments, a first acidic amino acid residue is at position P-2 (e.g., X²), a second acidic amino acid residue is positioned at P+1 (e.g., X⁵), a third acidic amino acid residue is positioned at P+2 (e.g., X⁶), a hydrophobic amino acid residue is positioned at P+4 (e.g., X⁸), a first aromatic amino acid residue is positioned at P+5 (e.g., X⁹), a second aromatic amino acid residue is positioned at P+8 (e.g., X¹²), and a third aromatic amino acid residue is positioned at P+9 (e.g., X¹³). In some embodiments, a stapled peptide agent comprises acidic amino acid residues at positions P-2 and P+1, and aromatic amino acid residues at positions P+5, P+8 and P+9. In some embodiments, a stapled peptide agent comprises acidic amino acid residues at positions P-2, P+1 and P+2, and aromatic amino acid residues at positions P+5, P+8 and P+9. In some embodiments, a stapled peptide agent comprises acidic amino acid residues at positions P-2 and P+1, a hydrophobic amino acid residue at position P+4, and aromatic amino acid residues at positions P+5, P+8 and P+9. In some embodiments, a stapled peptide agent comprises acidic amino acid residues at positions P-2, P+1 and P+2, a hydrophobic amino acid residue at position P+4, and aromatic amino acid residues at positions P+5, P+8 and P+9. In some embodiments, P is 3. In some embodiments, P is 4. In some embodiments, P is 5. In some embodiments, P is 6. In some embodiments, P is 7. In some embodiments, an amino acid residue at position P comprises two groups for stapling, e.g., B4, B5, B6, etc. In some embodiments, it is B4. In some embodiments, it is B5. In some embodiments, it is B6. In some embodiments, an agent comprises a staple and a first additional staple, e.g., a (i, i+3) or (i, i+4) staple. In some embodiments, a staple and a first additional staple are bonded to the same residue (e.g., B5, B6, etc.). In some embodiments, the other residue of a first additional residue is at position P-2 (e.g., when a moiety for stapling like a terminal olefin is in a P-terminal group which is considered a portion of X¹), P-3 or P-4. In some embodiments, an agent comprises a second additional staple, e.g., a (i, i+4) staple (e.g., stapling residues at positions P+6 (e.g., X¹⁰) and P+10 (e.g., X¹⁴), a (i, i+3) staple (e.g., stapling residues at positions P+3 (e.g., X⁷) and P+6 (e.g., X¹⁰), a (i, i+7) staple (e.g., stapling residues at positions P+3 (e.g., X⁷) and P+10 (e.g., X¹⁴), etc.). In some embodiments, an agent comprises a second additional staple which is a (i, i+4) staple stapling residues at positions P+6 (e.g., X¹⁰) and P+10 (e.g., X¹⁴). In some embodiments, an agent comprises a third additional staple, e.g., a (i, i+4) staple stapling residues at positions P-1 (e.g., X³) and P+3 (e.g., X⁷). In some embodiments, there are three staples in a stapled peptide agent. In some embodiments, there are four staples in a stapled peptide agent. As demonstrated herein, stapled agents comprising so positioned staples and residues can provide various desired properties and activities. In some embodiments, positioning of one or more staples may be shifted relevant to various acidic, hydrophobic and/or aromatic amino acid residues described herein, e.g., in some embodiments, stapled peptide agents comprise stapled residues at position P and P+7 (and optionally P-3 or P-4), acidic amino acid residues are at positions P-1, and P+2, and aromatic amino acid residues at positions P+6, P+9 and P+10, and optionally an acid amino acid residue at P+3 and/or a hydrophobic amino acid residue at position P+5. It was observed that various stapled peptide agents with shifted staples can bind to beta-catenin when assessed by fluorescence polarization.

Certain useful staples are described in the “Agents” section, below.

Certain stapled peptides are described in WO 2022/020652 and WO 2022/261257, the stapled peptides of each of which are independently incorporated herein by reference.

### Beta-Catenin

Among other things, the present disclosure provides technologies for modulating one or more beta-catenin functions. In some embodiments, the present disclosure provides useful technologies for inhibiting one or more beta-catenin functions that are associated with cancer or hyperplasia. In some embodiments, provided technologies are useful for preventing and treating conditions, disorders or diseases whose prevention and/or treatment will benefits from inhibition of beta-catenin. In some embodiments, a condition, disorder or disease is cancer.

Beta-catenin is reported to have various functions. For example, it can regulate and coordinate transcription of various genes. It is reported that high beta-catenin activity and/or expression levels may contribute to the development various conditions, disorders or diseases including cancer. Mutations and overexpression of beta-catenin are reported to be associated with conditions, disorders or diseases including many cancers including colorectal cancer, lung cancer, and breast cancer. Dysregulation of the Wnt/β-catenin signaling pathway has reportedly been linked to a number of conditions, disorders or diseases, including neurodegenerative diseases, psychiatric diseases, cancers, asthma, and even wound healing. An abundance of published research, both clinical and preclinical, has indicated that hyperactivated Wnt/beta-catenin activity drives tumorigenesis and is required for tumor maintenance in various cancers. Many Wnt inhibitors largely modulate this pathway at the extracellular ligand/receptor level, e.g., by preventing Wnt ligand secretion or by blocking Wnt ligand interaction with its receptors at the plasma membrane. It has been reported that many activating Wnt pathway mutations are found in APC and/or CTNNB1, which are downstream of membrane-proximal events. Among other things, the present disclosure encompasses the recognition that many agents at the extracellular ligand/receptor level are insufficient to treat many relevant patients, e.g., those with downstream mutations/abnormalities. In some embodiments, Wnt pathway-activating mutations converge on beta-catenin/TCF node. In some embodiments, the present disclosure targets beta-catenin/TCF interaction, e.g., as a therapeutic approach. Agents that can modulate beta-catenin functions are useful for various purposes including preventing and/or treating various conditions, disorders or diseases associated with beta-catenin.

In some embodiments, beta-catenin is in a mutant form which comprises one or more mutations. In some embodiments, a mutant form is associated with a condition, disorder or disease. For example, in some embodiments, a mutation is D32V. In some embodiments, a mutation is D32H. In some embodiments, a mutation is S45F. In some embodiments, a mutation is S37C. In some embodiments, an associated condition, disorder or disease is cancer, e.g., hepatocellular cancer.

### Binding Sites

Beta-catenin may interact with various agents at various binding sites each independently comprising a set of amino acid residues that interact with binding agents. For example, certain binding sites are utilized for beta-catenin interactions with Axin, APC, C-cadherin, E-cadherin, TCF3, and Bcl9. For interactions with TCF3, it has been reported that two or more binding sites may be utilized simultaneously to interact with different portions of TCF3. See, e.g., Graham et al. Cell, Vol. 103, 885-896, 2000.

In some embodiments, provided agents bind to beta-catenin at a unique binding site. In some embodiments, provided agents interact with beta-catenin at a set of amino acid residues that are different from previously reported binding sites, e.g., those for Axin, APC, C-cadherin, E-cadherin, TCF3 or Bcl9.

For example, in some embodiments, provided agents interact with one or more or all (e.g., about 1-23, 1-20, 1-15, 1-10, 1-5, 5-23, 5-20, 5-15, 5-10, 6-23, 6-20, 6-15, 6-10, 7-23, 7-20, 7-15, 7-10, 8-23, 8-20, 8-15, 8-10, 9-23, 9-20, 9-15, 9-10, 10-23, 10-20, 10-15, 11-23, 11-20, 11-15, 12-23, 12-20, 12-15, 13-23, 13-20, 13-15, 13-23, 14-20, 15-23, 15-20, 16-23, 16-20, 17-23, 17-20, 18-23, or 18-20, or about 1, 2, 3, 4, 5, 6, 7, 8, 9, 10, 11, 12, 13, 14, 15, 16, 17, 18, 19, 20, 21, 22, or 23, etc.) of a set of amino acid residues that are or correspond to amino acid residues in SEQ ID NO: 1, e.g., in some embodiments, the following amino acid residues of SEQ ID NO: 1: A305, Y306, G307, N308, Q309, K312, R342, K345, V346, V349, Q375, R376, Q379, N380, L382, W383, R386, N387, D413, N415, V416, T418, and C₄₁₉. In some embodiments, a set of amino acid residues are or correspond to amino acid residues A305, Y306, G307, N308, Q309, K312, R342, K345, V346, V349, Q375, Q379, N380, L382, W383, R386, N387, D413, N415, V416, T418, and C419 of SEQ ID NO: 1. In some embodiments, a set of amino acid residues are or correspond to amino acid residues A305, Y306, G307, N308, Q309, K312, K345, V346, V349, Q379, N380, L382, W383, R386, N387, D413, N415, V416, T418, and C419 of SEQ ID NO: 1. In some embodiments, a set of amino acid residues are or correspond to amino acid residues G307, K312, K345, W383, N387, D413, and N415 of SEQ ID NO: 1. In some embodiments, a set of amino acid residues are or correspond to amino acid residues G307, K312, K345, Q379, L382, W383, N387, N415 and V416 of SEQ ID NO: 1. In some embodiments, a set of amino acid residues are or correspond to amino acid residues Y306, G307, K312, K345, Q379, L382, W383, N387, N415 and V416 of SEQ ID NO: 1. In some embodiments, a set of amino acid residues are or correspond to amino acid residues G307, K312, K345, Q379, L382, W383, R386, N387, N415 and V416 of SEQ ID NO: 1. In some embodiments, a set of amino acid residues are or correspond to amino acid residues Y306, G307, K312, K345, Q379, L382, W383, R386, N387, N415 and V416 of SEQ ID NO: 1. In some embodiments, a set of amino acid residues are or correspond to amino acid residues Y306, G307, K312, K345, V349, Q379, L382, W383, N387, N415 and V416 of SEQ ID NO: 1. In some embodiments, a set of amino acid residues are or correspond to amino acid residues Y306, G307, K312, K345, V349, Q379, L382, W383, R386, N387, N415 and V416 of SEQ ID NO: 1. In some embodiments, a set of amino acid residues are or correspond to amino acid residues G307, K312, K345, W383, and N387 of SEQ ID NO: 1. In some embodiments, a set of amino acid residues are or correspond to amino acid residues Y306, G307, K312, R386 and N387 of SEQ ID NO: 1. In some embodiments, provided agents interact with Y306 or an amino acid residue corresponding thereto. In some embodiments, provided agents interact with G307 or an amino acid residue corresponding thereto. In some embodiments, provided agents interact with K312 or an amino acid residue corresponding thereto. In some embodiments, provided agents interact with K345 or an amino acid residue corresponding thereto. In some embodiments, provided agents interact with V349 or an amino acid residue corresponding thereto. In some embodiments, provided agents interact with Q379 or an amino acid residue corresponding thereto. In some embodiments, provided agents interact with L382 or an amino acid residue corresponding thereto. In some embodiments, provided agents interact with W383 or an amino acid residue corresponding thereto. In some embodiments, provided agents interact with R386 or an amino acid residue corresponding thereto. In some embodiments, provided agents interact with N387 or an amino acid residue corresponding thereto. In some embodiments, provided agents interact with N415 or an amino acid residue corresponding thereto. In some embodiments, provided agents interact with V416 or an amino acid residue corresponding thereto.

In some embodiments, a present agent interacts with a polypeptide whose sequence corresponds to aa 146-aa665 of human beta-catenin. In some embodiments, a present agent interacts with a polypeptide whose sequence comprises or is SEQ ID NO: 2:

(SEQ ID NO: 2)
SVLFYAITTLHNLLLHQEGAKMAVRLAGGLQKMVALLNKTNVKFL
AITTDCLQILAYGNQESKLIILASGGPQALVNIMRTYTYEKLLWT
TSRVLKVLSVCSSNKPAIVEAGGMQALGLHLTDPSQRLVQNCLWT
LRNLSDAATKQEGMEGLLGTLVQLLGSDDINVVTCAAGILSNLTC
NNYKNKMMVCQVGGIEALVRT.

In some embodiments, all amino acid residues that interact with a provided agent is with SEQ ID NO: 2. In some embodiments, amino acid residues that interact with a provided agent (e.g., one or more amino acid residues in an agent) interacts with an agent through hydrogen bonding, hydrophobic interactions or salt bridge. As appreciated by those skilled in the art, when two amino acid residues interacting with each other, they are typically within a certain range of distances when, e.g., assessed using crystallography, NMR, etc.

In some embodiments, certain amino acid residues reported to interact with one or more polypeptides are not significantly involved in interactions between provided and beta-catenin. In some embodiments, provided agents do not interact with an Axin binding site. In some embodiments, provided agents do not interact with a Bc19 binding site. In some embodiments, provided agents do not interact with one or more or all of amino acid residues that are or correspond to N426, C429, K435, R469, H470, S473, R474, K508 and N516 of SEQ ID NO: 1. In some embodiments, provided agents do not interact with N426 or an amino acid residue corresponding thereto. In some embodiments, provided agents do not interact with C429 or an amino acid residue corresponding thereto. In some embodiments, provided agents do not interact with K435 or an amino acid residue corresponding thereto. In some embodiments, provided agents do not interact with R469 or an amino acid residue corresponding thereto. In some embodiments, provided agents do not interact with H470 or an amino acid residue corresponding thereto. In some embodiments, provided agents do not interact with S473 or an amino acid residue corresponding thereto. In some embodiments, provided agents do not interact with R474 or an amino acid residue corresponding thereto. In some embodiments, provided agents do not interact with K508 or an amino acid residue corresponding thereto. In some embodiments, provided agents do not interact with N516 or an amino acid residue corresponding thereto.

In some embodiments, mutation of one or more amino acid residues outside of SEQ ID NO: 2 in beta-catenin does not significantly (e.g., not exceeding 30%, 40%, 50%, 60%, 70%, 75%, 80%, 85%, 90% or more) reduce interactions of beta-catenin with a provided agent. In some embodiments, mutation of one or more or all of amino acid residues that are or correspond to N426, C429, K435, R469, H470, S473, R474, K508 and N516 of SEQ ID NO: 1 does not significantly reduce interactions of beta-catenin with a provided agent. In some embodiments, mutation of N426 or an amino acid residue corresponding thereto does not significantly reduce interaction of beta-catenin with an agent. In some embodiments, mutation of Q379 or an amino acid residue corresponding thereto (e.g., to Ala, Glu, Phe, Trp, etc.) does not significantly reduce interaction of beta-catenin with an agent.

In some embodiments, an agent binds to a TCF site of beta-catenin. In some embodiments, an agent interacts with one or more but not all amino acid residues that interact with TCF. In some embodiments, an agent interacts with one or more but not all amino acid residues that interact with an extended region of XTcf3-CBD. In some embodiments, an agent does not interact with beta-catenin amino acid residues that interact with a beta-hairpin module of XTcf3-CBD. In some embodiments, an agent does not interact with beta-catenin amino acid residues that interact with a helix module of XTcf3-CBD. For certain amino acid residues that interact various modules of XTcF3-CBD, see, e.g., Graham et al. Cell, Vol. 103, 885-896, 2000.

In some embodiments, an agent competes with TCF for beta-catenin binding. In some embodiments, an agent competes with an extended region of TCF (e.g., Ala14-Glu24, or Asp16-Glu24, as described in Graham et al. Cell, Vol. 103, 885-896, 2000) for beta-catenin binding. In some embodiments, compared to an extended region of TCF, an agent does not compete, or competes at a less degree, with Axin for beta-catenin binding. In some embodiments, compared to an extended region of TCF, an agent does not compete, or competes at a less degree, with Bc19 for beta-catenin binding. In some embodiments, compared to an extended region of TCF, an agent does not compete, or competes at a less degree, with a beta-hairpin module of XTcf3-CBD for beta-catenin binding. In some embodiments, compared to an extended region of TCF, an agent does not compete, or competes at a less degree, with a helix module of XTcf3-CBD for beta-catenin binding. In some embodiments, an agent competes with E-cadherin for beta-catenin binding.

In some embodiments, the present disclosure provides complexes of peptides (e.g., polypeptides whose sequences are or comprises SEQ ID NO: 1 or 2) and provided agents. In some embodiments, in such complexes polypeptides and provided agents interact with one or more or all amino acid residues as described herein, and optionally do not interact with one or more or all amino acid residues as described herein.

In some embodiments, the present disclosure provides complexes comprising a provided agent and a beta-catenin polypeptide or a portion thereof. In some embodiments, a portion thereof comprises one or more or all of the interacting residues as described herein. In some embodiments, an agent and a beta-catenin polypeptide or a portion thereof interact with other at one or more or all of the interacting residues.

### Certain Agents

In some embodiments, the present disclosure provides an agent having the structure of formula I:

or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is an amino acid residue that comprises a side chain comprising an acidic or polar group;
L^{AA2} is an amino acid residue that comprises a side chain comprising an acidic or polar group;
L^{AA3} is an amino acid residue;
L^{AA4} is an amino acid residue that comprises a side chain comprising an optionally substituted aromatic group;
L^{AA5} is an amino acid residue that comprises a side chain comprising an optionally substituted aromatic group;
L^{AA6} is an amino acid residue that comprises a side chain comprising an optionally substituted aromatic group;
R^{C} is a peptide, a carboxyl protecting group, -L^{RC}-R′, —O-L^{RC}-R′ or —N(R′)-L^{RC}-R′; each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

In some embodiments, the present disclosure provides an agent having the structure of formula I:

or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is L^{AR}, wherein a methylene unit is replaced with —C(R′)(R^{AS})—, wherein R^{AS} is -L^{AS1}-R^{AA1}, wherein R^{AA1} is —CO₂R or —SO₂R;
L^{AA2} is L^{AR}, wherein a methylene unit is replaced with —C(R′)(R^{AS})—, wherein R^{AS} is -L^{AS2}-R^{AA2}, wherein R^{AA2} is —CO₂R or —SO₂R;
L^{AA3} is L^{AR}, wherein a methylene unit is replaced with —C(R′)(R^{AS})—, wherein R^{AS} is -L^{AS3}-R^{AA3}, wherein R^{AA3} is R′;
L^{AA4} is L^{AR}, wherein a methylene unit is replaced with —C(R′)(R^{AS})—, wherein R^{AS} is -L^{AS4}-R^{AA4}, wherein R^{AA4} is an optionally substituted group selected from 6-14 membered aryl or 5-14 membered heteroaryl having 1-6 heteroatoms;
L^{AA5} is L^{AR}, wherein a methylene unit is replaced with —C(R′)(R^{AS})—, wherein R^{AS} is -L^{AS5}-R^{AA5}, wherein R^{AA5} is an optionally substituted group selected from 6-14 membered aryl or 5-14 membered heteroaryl having 1-6 heteroatoms;
L^{AA6} is L^{AR}, wherein a methylene unit is replaced with —C(R′)(R^{AS})—, wherein R^{AS} is -L^{AS6}-R^{AA6}, wherein R^{AA6} is an optionally substituted group selected from 6-14 membered aryl or 5-14 membered heteroaryl having 1-6 heteroatoms;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L^{AR} is independently an optionally substituted, bivalent C₁-C₄ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each of L^{AS1}, L^{AS2}, L^{AS3}, L^{AS4}, LASS, and L^{AS6} is independently L^{AS};
each R^{AS} is independently -L^{AS}-R′;
each L^{AS} is independently an optionally substituted, bivalent C₁-C₁₀ aliphatic or heteroaliphatic group having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

In some embodiments, a compound of formula I is a stapled peptide as described herein.

Various agents, including embodiments for various variables, e.g., R′, R, R^{N}, L^{P1}, L^{AA1}, L^{P2}, L^{AA2}, L^{P3}, L^{AA3}, L^{P4}, L^{AA4}, L^{P5}, L^{AA5}, L^{P6}, L^{AA6}, L^{P7}, R^{C}, L^{AS}, etc., are described in WO 2022/261257 and incorporated herein by reference.

For example, in some embodiments, L^{P1} is or comprises -[X]ₚ-X¹—, wherein each of p, X and X¹ is independently as described herein, and X¹ is bonded to L^{AA1}. In some embodiments, L^{P1} is or comprises —X¹—. In some embodiments, such a carbon atom is an alpha carbon of an amino acid residue. In some embodiments, L^{AA1} is X² as described herein. In some embodiments, L^{P2} is or comprises -[X]pX⁴[X]p′-, wherein each of p, p′, X and X⁴ is independently as described herein. In some embodiments, L^{P2} is or comprises -[X]pX³X⁴[X]p′-, wherein each X, X³ and X⁴ is independently an amino acid residue, and each of p and p′ is independently 0-10. In some embodiments, L^{P2} is or comprises —X³X⁴—, wherein each X³ and X⁴ is independently as described herein, and X⁴ is bonded to L^{AA2}. In some embodiments, L^{AA2} is X⁵ as described herein. In some embodiments, L″3 is or comprises -[X]pX⁶X⁷[X]p′-, wherein each X, X⁶ and X⁷ is independently an amino acid residue, and each of p and p′ is independently 0-10. In some embodiments, L^{P3} is or comprises —X⁶X⁷—, wherein each X⁶ and X⁷ is independently an amino acid residue. In some embodiments, L^{AA3} is X⁸ as described herein. In some embodiments, L^{P4} is or comprises -[X]pX⁷X⁸[X]p′-, wherein each X, X⁷ and X⁸ is independently an amino acid residue, and each of p and p′ is independently 0-10. In some embodiments, L^{P4} is or comprises —X⁷X⁸—, wherein each X⁷ and X⁸ is independently as described herein, and X⁸ is bonded to L^{AA4}. In some embodiments, L^{AA4} is X⁹ as described herein. In some embodiments, L^{P5} is or comprises -[X]pX¹¹[X]p′-, wherein each variable is independently as described herein. In some embodiments, L^{P5} is or comprises —X¹⁰X¹¹—, wherein each X¹⁰ and X¹¹ is independently as described herein, and X¹¹ is bonded to LAAS. In some embodiments, L^{AA5} is or comprises amino acid residue. In some embodiments, L^{AA5} is or comprises an amino acid residue that comprises a side chain comprising an aromatic group. In some embodiments, L^{AA5} is X¹² as described herein. In some embodiments, L^{P6} is or comprises an amino acid residue. In some embodiments, L^{P6} is or comprises a peptide. In some embodiments, L^{AA6} is X¹³ as described herein. In some embodiments, L^{P7} is or comprises —X¹⁴—[X]p′-, wherein p′ is 0-10. In some embodiments, X¹⁴ is bonded to L^{AA6}

In some embodiments, an agent of formula I is a stapled peptide as described herein. In some embodiments, an agent of formula I is an agent selected from Table E2 or a pharmaceutically acceptable salt thereof. In some embodiments, an agent of formula I is an agent selected from Table E3 or a pharmaceutically acceptable salt thereof.

Among other things, the present disclosure provides agents, e.g. peptides, that can bind to beta-catenin. In some embodiments, an agent is or comprises X¹X²X³X⁴X⁵X⁶X⁷X⁸X⁹X¹⁰X¹¹X¹²X¹³X¹⁴ wherein each of X¹, X², X³, X⁴, X⁵, X⁶, X⁷, X⁸, X⁹, X¹⁰, X¹¹, X¹², X¹³ and X¹⁴ is independently an amino acid residue. In some embodiments, an agent is or comprises [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇, wherein each of p0, p15, p16 and p17 is independently 0 or 1, and each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

Various amino acid residues, e.g., those of formula A-I, A-II, A-III, A-IV, A-V, A-VI, PA, etc., can be utilized in accordance with the present disclosure. Certain useful amino acid residues are described in the present disclosure.

In some embodiments, each of X² and X⁵ is independently an acidic residue as described herein. In some embodiments, each of X², X⁵ and X⁶ is independently an acidic residue as described herein. In some embodiments, each of X⁹, X¹² and X¹³ are independently an amino acid residue comprising a side chain that comprises an aromatic group.

In some embodiments, X² is an acidic residue. In some embodiments, X² comprises a side chain that comprises —COOH or a derivative thereof. In some embodiments, X² comprises a side chain that comprises —COOH. In some embodiments, X² is Asp. Various other amino acid residues for X² are described else in the present disclosure.

In some embodiments, X⁵ is an acidic residue. In some embodiments, X⁵ comprises a side chain that comprises —COOH or a derivative thereof. In some embodiments, X⁵ comprises a side chain that comprises —COOH. In some embodiments, X⁵ is Asp. Various other amino acid residues for X⁵ are described else in the present disclosure.

In some embodiments, X⁶ is an acidic residue. In some embodiments, X⁶ comprises a side chain that comprises —COOH or a derivative thereof. In some embodiments, X⁶ comprises a side chain that comprises —COOH. In some embodiments, X⁶ is Asp. Various other amino acid residues for X⁶ are described else in the present disclosure.

In some embodiments, X⁹ comprises a side chain that comprises an aromatic group. In some embodiments, X⁹ comprises a side chain that comprises -R, wherein R is an optionally substituted group selected from phenyl, 10-membered bicyclic aryl, 5-membered heteroaryl having 1-3 hetereoatoms, and 9-10 membered bicyclic heteroaryl having 1-5 heteroatoms. In some embodiments, each heteroatom is independently sleeved from nitrogen, oxygen and sulfur. In some embodiments, X⁹ is Phe. Various other amino acid residues for X⁹ are described else in the present disclosure.

In some embodiments, X¹² comprises a side chain that comprises an aromatic group. In some embodiments, X¹² comprises a side chain that comprises -R, wherein R is an optionally substituted group selected from phenyl, 10-membered bicyclic aryl, 5-membered heteroaryl having 1-3 hetereoatoms, and 9-10 membered bicyclic heteroaryl having 1-5 heteroatoms. In some embodiments, each heteroatom is independently sleeved from nitrogen, oxygen and sulfur. In some embodiments, X¹² is 3Thi. In some embodiments, X¹² is 2F3McF. In some embodiments, X¹² is Phe. Various other amino acid residues for X¹² are described else in the present disclosure.

In some embodiments, X¹³ comprises a side chain that comprises an aromatic group. In some embodiments, X¹³ comprises a side chain that comprises -R, wherein R is an optionally substituted group selected from phenyl, 10-membered bicyclic aryl, 5-membered heteroaryl having 1-3 hetereoatoms, and 9-10 membered bicyclic heteroaryl having 1-5 heteroatoms. In some embodiments, each heteroatom is independently sleeved from nitrogen, oxygen and sulfur. In some embodiments, X¹³ is BtzA. In some embodiments, X¹³ is 34CIF. In some embodiments, X¹³ is 2NapA. Various other amino acid residues for X¹³ are described else in the present disclosure.

As described herein, in some embodiments, a peptide is a stapled peptide. In some embodiments, an agent is or comprises a peptide, wherein a peptide is a stapled peptide. In some embodiments, a peptide is a stitched peptide. In some embodiments, a peptide comprises three or more staples as described herein. In some embodiments, a peptide comprises three or more staples within a region having a length of, e.g., 11-15, such as 11, 14, etc., amino acid residues as described herein. In some embodiments, such a peptide provides improved rigidity, activity, delivery, solubility, and/or other desired properties comprising a reference peptide that is not stapled or that comprises fewer staples.

In some embodiments, the present disclosure provides an agent, e.g., a peptide, comprising X¹X²X³X⁴X⁵X⁶X⁷X⁸X⁹X¹⁰X¹¹X¹²X¹³X¹⁴, wherein X¹, X², X³, X⁴, X⁵, X⁶, X⁷, X⁸, X⁹, X¹⁰, X¹¹, X¹², X¹³, and X¹⁴ are each independently an amino acid residue and comprises two or more pairs of amino acid residues, wherein each pair of amino acid residues are independently two amino acid residues suitable for stapling or stapled. In some embodiments, the present disclosure provides an agent, e.g., a peptide, comprising X¹X²X³X⁴X⁵X⁶X⁷X⁸X⁹X¹⁰X¹¹X¹²X¹³X¹⁴, wherein X¹, X², X³, X⁴, X⁵, X⁶, X⁷, X⁸, X⁹, X¹⁰, X¹¹, X¹², X¹³, and X¹⁴ are each independently an amino acid residue and comprises two or more pairs of amino acid residues, wherein each pair of amino acid residues are independently three amino acid residues suitable for stapling or stapled.

In some embodiments, the present disclosure provides an agent, e.g., a peptide, comprising [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇, wherein each of p0, p15, p16 and p17 is independently 0 or 1, and X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ are each independently an amino acid residue and comprises two or more pairs of amino acid residues, wherein each pair of amino acid residues are independently two amino acid residues suitable for stapling or stapled. In some embodiments, the present disclosure provides an agent, e.g., a peptide, comprising [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇, wherein each of p0, p15, p16 and p17 is independently 0 or 1, and X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ are each independently an amino acid residue and comprises three or more pairs of amino acid residues, wherein each pair of amino acid residues are independently two amino acid residues suitable for stapling or stapled. In some embodiments, each amino acid residue in such pairs of amino acid residues are independently selected from X¹, X², X³, X⁴, X⁵, X⁶, X⁷, X⁸, X⁹, X¹⁰, X¹¹, X¹², X¹³, and X¹⁴.

In some embodiments, there are three such pairs of amino acid residues. In some embodiments, there are four such pairs of amino acid residues. In some embodiments, there are four or more such pairs of amino acid residues. In some embodiments, each pair is independently not stapled. In some embodiments, one or more pairs are independently stapled. In some embodiments, two or more pairs are independently stapled. In some embodiments, three or more pairs are independently stapled. In some embodiments, four or more pairs are independently stapled. In some embodiments, two pairs are independently stapled. In some embodiments, three pairs are independently stapled. In some embodiments, four pairs are independently stapled.

In some embodiments, a pair is X¹ and X⁴. In some embodiments, a pair is X⁴ and X¹¹. In some embodiments, a pair is X¹ and X³. In some embodiments, a pair is X⁴ and X¹¹. In some embodiments, a pair is X¹⁰ and X¹⁴. In some embodiments, a pair is X⁷ and X¹⁰. In some embodiments, a pair is X⁷ and X¹⁴. In some embodiments, a pair is X³ and X⁷.

In some embodiments, a pair is X¹ and X¹⁴ and a pair is X⁴ and X¹¹. In some embodiments, a pair is X¹ and X¹⁴, a pair is X⁴ and X¹¹ and a pair is X¹⁰ and X¹⁴. In some embodiments, a pair is X¹ and X¹⁴, a pair is X⁴ and X¹¹ and a pair is X⁷ and X¹⁰. In some embodiments, a pair is X¹ and X¹⁴, a pair is X⁴ and X¹¹ and a pair is X⁷ and X¹⁴. In some embodiments, a pair is X¹ and X¹⁴, a pair is X⁴ and X¹¹, a pair is X³ and X⁷, and a pair is X⁷ and X¹⁴. In some embodiments, each pair is independently a pair of amino acid residues suitable for stapling. In some embodiments, each pair is independently stapled.

In some embodiments, a pair is X¹ and X³, a pair is X⁴ and X¹¹, and a pair is X¹⁰ and X¹⁴. In some embodiments, each pair is independently a pair of amino acid residues suitable for stapling. In some embodiments, each pair is independently stapled.

In some embodiments, the present disclosure provides an agent, which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein the agent binds to beta-catenin.

In some embodiments, X² comprises a side chain comprising an acidic or a polar group. In some embodiments, X² comprises a side chain comprising an acidic group. In some embodiments, X² comprises a side chain comprising a polar group. In some embodiments, X⁵ comprises a side chain comprising an acidic or a polar group. In some embodiments, X⁵ comprises a side chain comprising an acidic group. In some embodiments, X⁵ comprises a side chain comprising a polar group. In some embodiments, X¹³ comprises a side chain comprising an optionally substituted aromatic group. In some embodiments, two or more of X¹, X³, X⁴, X⁷, X¹⁰, X¹¹ and X¹⁴ are each independently an amino acid residue suitable for stapling, or are each independently stapled.

In some embodiments, the present disclosure provides an agent, which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled. In some embodiments, three or more of X¹, X³, X⁴, X⁷, X¹⁰, X¹¹ and X¹⁴ are each independently an amino acid residue suitable for stapling, or are each independently stapled. In some embodiments, four or more of X¹, X³, X⁴, X⁷, X¹⁰, X¹¹ and X¹⁴ are each independently an amino acid residue suitable for stapling, or are each independently stapled. In some embodiments, five of X¹, X³, X⁴, X⁷, X¹⁰, X¹¹ and X¹⁴ are each independently an amino acid residue suitable for stapling, or are each independently stapled. In some embodiments, X¹ and X⁴ are each independently an amino acid residue suitable for stapling. In some embodiments, X¹ and X³ are each independently an amino acid residue suitable for stapling. In some embodiments, X⁴ and X¹¹ are each independently an amino acid suitable for stapling. In some embodiments, X¹, X⁴, and X¹¹ are each independently an amino acid residue suitable for stapling. In some embodiments, X¹⁰ and X¹⁴ are each independently an amino acid residue suitable for stapling. In some embodiments, X⁷ and X¹⁰ are each independently an amino acid residue suitable for stapling. In some embodiments, X⁷ and X¹⁴ are each independently an amino acid residue suitable for stapling. In some embodiments, X³ and X⁷ are each independently an amino acid residue suitable for stapling. In some embodiments, X¹ and X⁴ are connected by a staple. In some embodiments, X¹ and X³ are connected by a staple. In some embodiments, X⁴ and X¹¹ are connected by a staple. In some embodiments, X¹ and X⁴ connected by a staple, and X⁴ and X¹¹ are connected by a staple. In some embodiments, X¹⁰ and X¹⁴ are connected by a staple. In some embodiments, X⁷ and X¹⁰ are connected by a staple. In some embodiments, X⁷ and X¹⁴ are connected by a staple. In some embodiments, X³ and X⁷ are connected by a staple.

In some embodiments, the present disclosure provides an agent, which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple, and X¹⁰ and X¹⁴ are connected by a staple.

In some embodiments, the present disclosure provides an agent, which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple, and X⁷ and X¹⁰ are connected by a staple.

In some embodiments, the present disclosure provides an agent, which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple, and X⁷ and X¹⁴ are connected by a staple.

In some embodiments, the present disclosure provides an agent, which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X⁴ are connected by a staple and/or X⁴ and X¹¹ are connected by a staple; and X¹⁰ and X¹⁴ are connected by a staple and/or X³ and X⁷ are connected by a staple.

In some embodiments, the present disclosure provides an agent, which is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and wherein:
X¹ and X³ are connected by a staple, X⁴ and X¹¹ are connected by a staple; and X¹⁰ and X¹⁴ are connected by a staple.

In some embodiments, X² comprises a side chain comprising an acidic (e.g., —COOH) or a polar group. In some embodiments, X² comprises a side chain comprising an acid group. In some embodiments, X⁵ comprises a side chain comprising an acidic or a polar group. In some embodiments, X⁵ comprises a side chain comprising an acid group. In some embodiments, X⁶ comprises a side chain comprising an acidic or a polar group. In some embodiments, X⁶ comprises a side chain comprising an acid group. In some embodiments, X⁹ comprises a side chain comprising an optionally substituted aromatic group. In some embodiments, X¹² comprises a side chain comprising an optionally substituted aromatic group. In some embodiments, X¹³ comprises a side chain comprising an optionally substituted aromatic group. In some embodiments, X² and X⁵ each independently comprise a side chain comprising an acidic or a polar group. In some embodiments, X² and X⁶ each independently comprise a side chain comprising an acidic or a polar group. In some embodiments, X⁵ and X⁶ each independently comprise a side chain comprising an acidic or a polar group. In some embodiments, X² and X⁵ each independently comprise a side chain comprising an acidic group. In some embodiments, X² and X⁶ each independently comprise a side chain comprising an acidic group. In some embodiments, X⁵ and X⁶ each independently comprise a side chain comprising an acidic group. In some embodiments, X², X⁵ and X⁶ each independently comprise a side chain comprising an acidic or a polar group. In some embodiments, X², X⁵ and X⁶ each independently comprise a side chain comprising an acidic group. In some embodiments, each of X⁹ and X¹² independently comprises a side chain comprising an optionally substituted aromatic group. In some embodiments, each of X⁹ and X¹³ independently comprises a side chain comprising an optionally substituted aromatic group. In some embodiments, each of X⁹, X¹² and X¹³ independently comprises a side chain comprising an optionally substituted aromatic group. In some embodiments, each of X² and X⁵ independently comprises a side chain comprising an acidic group (e.g., —COOH), and each of X⁹, X¹² and X¹³ independently comprises a side chain comprising an optionally substituted aromatic group. In some embodiments, each of X², X⁵ and X⁶ independently comprises a side chain comprising an acidic group (e.g., —COOH), and each of X⁹, X¹² and X¹³independently comprises a side chain comprising an optionally substituted aromatic group.

As described herein, various types of amino acid residues (e.g., those of amino acids having the structure of formula A-I, A-II, A-III, A-IV, A-V, A-VI, etc. as described herein or in WO 2022/261257 and incorporated herein by reference) can be utilized in accordance with the present disclosure. Certain examples are described herein for X⁰, X¹, X², X³, X⁴, X⁵, X⁶, X⁷, X⁸, X⁹, X¹⁰, X¹¹, X¹², X¹³, X¹⁴, X¹⁵, X¹⁶, X¹⁷, etc.

Various embodiments are described for p0, X⁰, X¹, X², X³, X⁴, X⁵, X⁶, X⁷, X⁸, X⁹, X¹⁰, X¹¹, X¹², X¹³, X¹⁴, X¹⁵, X¹⁶, X¹⁷, etc. in WO 2022/261257 and are incorporated by reference.

For example, in some embodiments, X¹ is an amino acid reside suitable for stapling. In some embodiments, X¹ is stapled. In some embodiments, X¹ is PL3. In some embodiments, -Cy- is optionally substituted phenylene. PL3. In some embodiments, X² is a residue of amino acid (e.g., of formula A-I, A-II, A-III, A-IV, A-V, A-VI, etc. or a salt thereof) that comprises an acidic or polar group. In some embodiments, X² is a residue of amino acid whose side chain comprises an acidic group (in some embodiments, may be referred to as an “acidic amino acid residue”). In some embodiments, X² is Asp. In some embodiments, X² is Glu. In some embodiments, X³ is a hydrophobic amino acid residue. In some embodiments, X³ is a residue of an amino acid whose side chain is C₁₋₁₀ aliphatic optionally substituted with one or more non-polar and non-charged groups. In some embodiments, it is a residue of an amino acid whose side chain is C₁₋₁₀ aliphatic optionally substituted with one or more hydrophobic substituents. In some embodiments, it is a residue of an amino acid whose side chain is C₁₋₁₀ aliphatic. In some embodiments, it is a residue of an amino acid whose side chain is C₁₋₁₀ alkyl. In some embodiments, X³ is a residue of Ala, Val, Ile, Leu, Met, Phe, Tyr, Trp, Npg, Leu, Cha, Val, nLeu, Ile, CypA, CyLeu, Chg, DiethA, Ala, Aib, OctG, or Cba. In some embodiments, X³ is a residue of Npg, Leu, or Cha. In some embodiments, X³ is a residue of Npg. In some embodiments, X³ is a residue of Leu. In some embodiments, X³ is a residue of Cha. In some embodiments, X³ is a residue of Val. In some embodiments, X³ is a residue of nLeu. In some embodiments, X³ is a residue of Ile. In some embodiments, X³ is a residue of CypA. In some embodiments, X³ is a residue of CyLeu. In some embodiments, X³ is a residue of Chg. In some embodiments, X³ is a residue of DicthA. In some embodiments, X³ is a residue of Ala. In some embodiments, X³ is a residue of Aib. In some embodiments, X³ is a residue of OctG. In some embodiments, X³ is a residue of Cba. In some embodiments, X⁴ is residue of an amino acid suitable for stapling. In some embodiments, X⁴ is a residue of an amino acid which comprises two functional groups suitable for stapling. In some embodiments, X⁴ is a residue of an amino acid which comprises two olefins, e.g., two terminal olefins. In some embodiments, X⁴ is stapled. In some embodiments, X⁴ is stapled with X¹ and/or X¹¹. In some embodiments, X⁴ is B5. In some embodiments, X⁴ is R5. In some embodiments, X⁴ is R4. In some embodiments, X⁴ is R6. In some embodiments, X⁴ is S5. In some embodiments, X⁶ is a residue of amino acid that comprises an acidic or polar group. In some embodiments, X⁶ is a residue of amino acid whose side chain comprises an acidic group, e.g., a —COOH group or a salt form thereof. In some embodiments, X⁶ is a residue of 3COOHF, TfeGA, Asp, [CH2CMe2CO2H]TriAzDap, Glu, 2OH3COOHF, 4OH3COOHF, 4COOHF, 2COOHF, 5F3Me2COOHF, 4F3Me2COOHF, 5F3Me3COOHF, 4F3Me3COOHF, 3F2COOHF, or dGlu. In some embodiments, X⁶ is a residue of 3COOHF, TfeGA, Asp, or [CH₂CMe2CO₂H]TriAzDap. In some embodiments, X⁶ is a residue of 3COOHF. In some embodiments, X⁶ is a residue of TfeGA. In some embodiments, X⁶ is a residue of Asp. In some embodiments, X⁶ is a residue of Glu. In some embodiments, X⁷ is a hydrophobic amino acid residue, e.g., those described for X³. In some embodiments, X⁷ is a residue of an amino acid whose side chain is C₁₋₁₀ aliphatic optionally substituted with one or more non-polar and non-charged groups. In some embodiments, X⁷ is a residue of Ala. In some embodiments, X⁷ is a residue of Aib. In some embodiments, X⁷ is a residue of nLeu. In some embodiments, X⁷ is a residue of Cha. In some embodiments, X⁷ is a residue of Npg. In some embodiments, X⁷ is a residue of sAla. In some embodiments, X⁷ is a residue of Val. In some embodiments, X⁷ is a residue of CyLeu. In some embodiments, X⁷ is a residue of Leu. In some embodiments, X⁷ is a residue of Cpg. In some embodiments, X⁷ is a residue of Cbg. In some embodiments, X⁷ is a residue of aMeL. In some embodiments, X⁷ is a residue of DaMeL. In some embodiments, X⁷ is a residue of aMeV. In some embodiments, X⁸ is a hydrophobic amino acid residue, e.g., those described for X³. In some embodiments, X⁸ is a residue of Ala. In some embodiments, X⁸ is a residue of Aib. In some embodiments, X⁸ is a residue of Cpg. In some embodiments, X⁸ is a residue of Val. In some embodiments, X⁸ is a residue of Leu. In some embodiments, X⁸ is a residue of nLeu. In some embodiments, X⁸ is a residue of Cba. In some embodiments, X⁹ comprises a side chain comprising an optionally substituted aromatic group. In some embodiments, X⁹ is an aromatic amino acid residue as described herein. In some embodiments, X⁹ is Phe. In some embodiments, X⁹ is 3COOHF. In some embodiments, X⁹ is 2NapA. In some embodiments, X⁹ is Tyr. In some embodiments, X⁹ is 3Thi. In some embodiments, X⁹ is 4FF. In some embodiments, X⁹ is 4CIF. In some embodiments, X⁹ is 4BrF. In some embodiments, X⁹ is 3FF. In some embodiments, X⁹ is 3CIF. In some embodiments, X⁹ is 3BrF. In some embodiments, X⁹ is 2FF. In some embodiments, X⁹ is 30MeF. In some embodiments, X⁹ is 4CNF. In some embodiments, X⁹ is 3CNF. In some embodiments, X⁹ is 4MeF. In some embodiments, X⁹ is 3MeF. In some embodiments, X¹⁰ is a residue of an amino acid whose side chain comprises a polar group. In some embodiments, X¹⁰ is Asn, Gln, Lys, etc. In some embodiments, X¹⁰ is stapled. In some embodiments, X¹⁰ is not stapled. In some embodiments, X¹⁰ is stapled with X¹⁴. In some embodiments, a staple comprises —C(O)NH—. In some embodiments, X¹¹ is a residue of an amino acid suitable for stapling as described herein. In some embodiments, X¹¹ comprises —CH═CH₂. In some embodiments, X¹¹ is a residue of PyrS2. In some embodiments, X¹¹ is Az3. In some embodiments, X¹¹ is PyrS1. In some embodiments, X¹² comprises a side chain comprising an optionally substituted aromatic group. In some embodiments, X¹² is an aromatic amino acid residue as described herein. In some embodiments, X¹² is a residue of an amino acid selected from Phe, 1NapA, 2cmbF, 2F3MeF, 2CIF, 2FurA, 20MeF, 2MeF, 2BrF, 2CNF, 2NO₂F, 2PyrA, 2Thi, 3PyrA, 3Thi, or 4PyrA. In some embodiments, X¹² is a residue of Phe. In some embodiments, X¹² is a residue of 2F3MeF. In some embodiments, X¹² is a residue of 2CIF. In some embodiments, X¹² is a residue of 2FurA. In some embodiments, X¹² is a residue of 20MeF. In some embodiments, X¹² is a residue of 2MeF. In some embodiments, X¹² is a residue of 2BrF. In some embodiments, X¹² is a residue of 2CNF. In some embodiments, X¹² is a residue of 2NO₂F. In some embodiments, X¹² is a residue of 2PyraA. In some embodiments, X¹² is a residue of 3PyrA. In some embodiments, X¹² is a residue of 3Thi. In some embodiments, X¹² is a residue of 4PyrA. In some embodiments, X¹² is a residue of His. In some embodiments, X¹² is a residue of 1NapA. In some embodiments, X¹² is a residue of 2Thi. In some embodiments, X¹² is a residue of 2cmbF. In some embodiments, X¹³ comprises a side chain which is or comprises an optionally substituted aromatic group. In some embodiments, X¹³ is an aromatic amino acid residue as described herein. In some embodiments, X¹³ is a residue of BztA, 34CIF, 3Thi, Phe, 34MeF, 3BrF, 7FBztA, 2BrF, 3F3MeF, 4F3MeF, RbMe2NapA, RbMeBzta, SbMeBzta, 5IndA, 7CIBztA, 7MeBztA, 2CIF, 3CIF, 4BrF, 4CIF, or 3MeF. In some embodiments, X¹³ is BztA. In some embodiments, X¹³ is 34CIF. In some embodiments, X¹³ is 3Thi. In some embodiments, X¹³ is Phe. In some embodiments, X¹⁴ is an amino acid residue suitable for stapling. In some embodiments, X¹⁴ is stapled. In some embodiments, X¹⁴ is stapled with X¹⁰ as described herein. In some embodiments, X¹⁴ is stapled with X⁷ as described herein. In some embodiments, X¹⁴ is GlnR, Lys, sAla, Gln, Cys, TriAzLys, AsnR, hGlnR, 4PipA, sAbu, Orn, dGlnR, [4mampiperidine]GlnR, [39N2spiroundecane]GlnR, [29N2spiroundecane]GlnR, iPrLys, sCH2S, [diaminobutane]GlnR, or [4aminopiperidine]GlnR. In some embodiments, X¹⁴ is GlnR. In some embodiments, X¹⁴ is Lys. In some embodiments, X¹⁴ is sAla. In some embodiments, X¹⁴ is Gln. In some embodiments, X¹⁴ is Cys. In some embodiments, X¹⁴ is TriAzLys. In some embodiments, X¹⁴ is AsnR. In some embodiments, X¹⁴ is hGlnR. In some embodiments, X¹⁴ is 4PipA. In some embodiments, X¹⁴ is sAbu. In some embodiments, X¹⁴ is Orn. In some embodiments, X¹⁴ is dGlnR. In some embodiments, X¹⁴ is [4mampiperidine]GlnR. In some embodiments, X¹⁴ is [39N2spiroundecane]GlnR. In some embodiments, X¹⁴ is [29N2spiroundecane]GlnR. In some embodiments, X¹⁴ is iPrLys. In some embodiments, X¹⁴ is sCH2S. In some embodiments, X¹⁴ is [diaminobutane]GlnR. In some embodiments, X¹⁴ is [4aminopiperidine]GlnR. In some embodiments, X¹⁴ is a C-terminus amino acid residue. In some embodiments, p15 is 1. In some embodiments, p15 is 0. In some embodiments, X¹⁵ is a residue of Ala, Leu, Val, Aib, MorphNva, Thr, dAla, dLcu, [BiotinPEG8]Lys, Glu, or AzLys. In some embodiments, p16 is 1. In some embodiments, p16 is 0. In some embodiments, p17 is 1. In some embodiments, p17 is 0. In some embodiments, p18 is 1. In some embodiments, p18 is 0. In some embodiments, p19 is 1. In some embodiments, p19 is 0. In some embodiments, p20 is 1. In some embodiments, p20 is 0. In some embodiments, p21 is 1. In some embodiments, p21 is 0. In some embodiments, p22 is 1. In some embodiments, p22 is 0. In some embodiments, p23 is 1. In some embodiments, p23 is 0.

In some embodiments, an agent is or comprises a peptide having the structure of:

or a salt thereof, wherein:
each X is independently an amino acid residue;
each p and p′ is independently 0-10;
R^{N} is independently a peptide, an amino protecting group or R′-L^{RN}-;
R^{C} is independently a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, p is 0. In some embodiments, p is 1, 2, 3, 4, 5, 6, 7, 8, 9, or 10. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, p′ is 0. In some embodiments, p′ is 1, 2, 3, 4, 5, 6, 7, 8, 9, or 10. In some embodiments, p′ is 1. In some embodiments, p′ is 2. In some embodiments, p′ is 3. In some embodiments, p′ is 4. In some embodiments, p′ is 5. In some embodiments, p′ is 6. In some embodiments, p′ is 7. In some embodiments, p′ is 8. In some embodiments, p′ is 9. In some embodiments, p′ is 10.

In some embodiments, R^{N} is an N-terminus capping group. In some embodiments, R^{N} is —C(O)R, wherein R is as described herein. In some embodiments, R is —H. In some embodiments, R is optionally substituted C₁₋₆ aliphatic. In some embodiments, R is optionally substituted C₁₋₆ alkyl. In some embodiments, R is methyl. In some embodiments, R^{N} is Ac. In some embodiments, R^{N} is a group suitable for stapling, or is stapled. In some embodiments, R^{N} is 4pentenyl. In some embodiments, R^{N} is 5hexenyl. In some embodiments, R^{N} is BzAm20Allyl. In some embodiments, R^{N} is Ac, NPyroR3, 5hexenyl, 4pentenyl, Bua, C3a, Cpc, Cbc, CypCO, Bnc, CF3CO, 2PyCypCO, 4THPCO, Isobutyryl, Ts, 15PyraPy, 2PyBu, 4PymCO, 4PyPrpc, 3IAPAc, 4MePipzPrpC, MePipAc, Melmid4SO2, BzAm20Allyl, Hex, 2PyzCO, 3Phc3, MeOPr, lithocholate, 2FPhc, PhC, MeSO2, Isovaleryl, EtHNCO, TzPyr, 8IAP, 3PydCO, 2PymCO, 5PymCO, 1Imidac, 2F2PyAc, 2IAPAc, 124TriPr, 6QuiAc, 3PyAc, 123TriAc, 1PyrazoleAc, 3PyPrpc, 5PymAc, 1PydoneAc, 124TriAc, Me2NAc, 8QuiSO2, MPEG4, mPEG8, mPEG16 or mPEG24.

In some embodiments, R^{C} is a C-terminus capping group. In some embodiments, R^{C} comprises a PEG moiety. In some embodiments, R^{C} is —O-L^{RC}-R′ wherein each of L^{RC} and R′ is independently as described herein. In some embodiments, R^{C} is —OR′, wherein R′ is optionally substituted C₁-C₁₀ aliphatic. In some embodiments, R^{C} is —OR′, wherein R′ is optionally substituted C₁-C₁₀ alkyl. In some embodiments, R^{C} is —OR′, wherein R′ is optionally substituted C₁₋₃₀ heteroaliphatic having 1-10 heteroatoms. In some embodiments, R^{C} is -L^{RC}-R′, wherein one or more methylene unit of L^{RC} are replaced with —O— and R′ is as described herein. In some embodiments, R^{C} is —O-L^{RC}-R′, wherein L^{RC} is optionally substituted C₁₋₆ alkylene and R′ is as described herein. In some embodiments, R^{C} is —O-L^{RC}-R′, wherein L^{RC} is optionally substituted C₁₋₆ alkylene and R′ is an optionally substituted group selected from C₆₋₃₀ aryl and 5-30 membered heteroaryl having 1-10 heteroatoms. In some embodiments, R^{C} is —OR′ wherein R′ is optionally substituted C₆₋₃₀ arylaliphatic. In some embodiments, R^{C} is —N(R′)₂ wherein each R′ is independently as described herein. In some embodiments, R^{C} is —NHR′ wherein R′ is as described herein. In some embodiments, R^{C} is —N(R)₂ wherein each R is independently as described herein. In some embodiments, R^{C} is —NHR wherein R is as described herein. In some embodiments, R is —H. In some embodiments, R is optionally substituted C₁₋₆ aliphatic. In some embodiments, R is optionally substituted C₁₋₆ alkyl. In some embodiments, R is methyl. In some embodiments, R is ethyl. In some embodiments, R^{C} is —NH₂. In some embodiments, R^{C} is —NHEt.

In some embodiments, R^{C} is —NHC (CH₃)CH₂OH. In some embodiments, R^{C} is —(S)—NHC (CH₃)CH₂OH. In some embodiments, R^{C} is —(R)—NHC (CH₃)CH₂OH. In some embodiments, R^{C} is

In some embodiments, R^{C} is

In some embodiments, R^{C} is

In some embodiments, R^{C} is

In some embodiments, R^{C} is

In some embodiments, R^{C} is -Alaol, wherein the amino group of -Alaol is bonded to the last —C(O)— of the peptide backbone (R^{C} is

In some embodiments, R^{C} is -dAlaol, wherein the amino group of -dAlaol is bonded to the last —C(O)— of the peptide backbone (R^{C} is

In some embodiments, R^{C} is -Prool, wherein the amino group of -Prool is bonded to the last —C(O)— of the peptide backbone (R^{C} is

In some embodiments, R^{C} is -Throl, wherein the amino group of -Throl is bonded to the last —C(O)— of the peptide backbone (R^{C} is

In some embodiments, R^{C} is -Serol, wherein the amino group of -Serol is bonded to the last —C(O)— of the peptide backbone (R^{C} is

In some embodiments, R^{C} is —OH.

In some embodiments, an agent is or comprises a peptide analog. In some embodiments, an agent is or comprises an analog of a peptide described herein or a portion thereof. In some embodiments, an agent is or comprises an analog of a peptide described herein. In some embodiments, in a peptide analog, one or more of amino (e.g., —NH—), carbonyl or amide group (e.g., —C(O)NH—) are independently replaced with LR each of which is independently L as described herein. In some embodiments, in an analog of a peptide, one or more of backbone amino, carbonyl or amide group are independently replaced with LR as described herein. In some embodiments, LR is a bivalent moiety with a length of 0, 1, 2, 3, 4, or 5 atoms between its two covalent bonds (e.g., for —CH₂—, one atom (one carbon atom between the two covalent bonds); for —CH₂CH₂—, two atoms (two carbon atoms between the two covalent bonds), etc.). In some embodiments, a length is 1 atom. In some embodiments, a length is 2 atoms. In some embodiments, a length is 3 atoms. In some embodiments, a length is 4 atoms. In some embodiments, a length is 5 atoms. In some embodiments, the length of L^{R} (or L, or any moiety that can be L), measured by the number of atoms between its two covalent bonds, are the same as, or no more than about 1 or 2 atoms longer or shorter than, a group it replaces. In some embodiments, the length of L^{R} (or L, or any moiety that can be L), measured by the number of atoms between its two covalent bonds, are the same as a group it replaces. In some embodiments, L^{R} is a covalent bond. In some embodiments, L^{R} is optionally substituted —CH₂—. In some embodiments, L^{R} is optionally substituted —CH₂—CH₂—. In some embodiments, L^{R} is optionally substituted —CHR′—CHR′—. In some embodiments, L^{R} is optionally substituted —C(R′)₂—C(R′)₂—. In some embodiments, L^{R} is optionally substituted —CH═CH—. In some embodiments, L^{R} is —C(R′)═C(R′)—. In some embodiments, L is L^{R} as described herein. In some embodiments, an amino group is replaced with a covalent bond. In some embodiments, an amino group is replaced with optionally substituted —CH₂—. In some embodiments, a carbonyl group is replaced with a covalent bond. In some embodiments, a carbonyl group is replaced with optionally substituted —CH₂—. In some embodiments, an amide group is replaced with a covalent bond. In some embodiments, an amide group is replaced with optionally substituted —CH₂—. In some embodiments, an amide group is replaced with optionally substituted —CH₂—CH₂—. In some embodiments, an amide group is replaced with —C(R′)₂—C(R′)₂—. In some embodiments, an amide group is replaced with optionally substituted —CHR′—CHR′—. In some embodiments, an amide group is replaced with optionally substituted —CH═CH—. In some embodiments, an amide group is replaced with —C(R′)═C(R′)—.

### Amino Acids

As appreciated by those skilled in the art, various amino acids may be utilized in accordance with the present disclosure. For example, both naturally occurring and non-naturally occurring amino acids can be utilized in accordance with the present disclosure. In some embodiments, an amino acid is a compound comprising an amino group that can form an amide group with a carboxyl group and a carboxyl group. In some embodiments, an amino acid is an alpha amino acid. In some embodiments, an amino acid is a beta-amino acid. In some embodiments, an amino acid is a D-amino acid. In some embodiments, an amino acid is a L-amino acid. In some embodiments, an amino acid is an naturally encoded amino acid, e.g., in mammalian cells.

In some embodiments, an amino acid is a compound having the structure of formula A-I: or a salt thereof, wherein:

each of R^{a1}, R^{a2}, R^{a3} is independently -L^{a}-R′;
each of L^{a}, L^{a1} and L^{a2} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a compound having the structure of formula A-I or a salt thereof has the structure of NH(R^{a1})-L^{a1}-C(R^{a2})(R^{a3})-L^{a2}—COOH or a salt thereof.

In some embodiments, a ring moiety of, e.g., -Cy-, R (including those formed by R groups taken together), etc. is monocyclic. In some embodiments, a ring moiety is bicyclic or polycyclic. In some embodiments, a monocyclic ring is an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms. In some embodiments, each monocyclic ring unit of a bicyclic or polycyclic ring moiety is independently an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms.

In some embodiments, each heteroatom is independently selected from oxygen, nitrogen, sulfur, phosphorus and silicon. In some embodiments, each heteroatom is independently selected from oxygen, nitrogen, and sulfur.

In some embodiments, L^{a1} is a covalent bond. In some embodiments, a compound of formula A-1 is of the structure NH(R^{a1})—C(R^{a2})(R^{a3})-L^{a2}—COOH.

In some embodiments, L^{a2} is a covalent bond. In some embodiments, a compound of formula A-1 is of the structure NH(R^{a1})—C(R^{a2})(R^{a3})-L^{a2}—COOH).

In some embodiments, L^{a1} is a covalent bond and L^{a2} is a covalent bond. In some embodiments, a compound of formula A-1 is of the structure NH(R^{a1})—C(R^{a2})(R^{a3})—COOH.

In some embodiments, an amino acid is suitable for stapling. In some embodiments, an amino acid comprises a terminal olefin. Certain such amino acids are exemplified herein (e.g., those described in or utilized in peptides of various Tables).

In some embodiments, an agent comprises a detectable moiety, which can either be detected directly or indirectly. For example, in some embodiments, a detectable moiety is or comprises a fluorescent group. In some embodiments, a detectable moiety is or comprises a biotin moiety. In some embodiments, a detectable moiety is connected to the rest of an agent at an amino acid residue, e.g., through a side chain, optionally through a linker (e.g., L as described herein). In some embodiments, a detectable moiety is —N3, which may be detected after a click chemistry reaction with a labeled agent comprising an alkyne.

In some embodiments, the present disclosure provides various compounds, which among other things may be utilized as amino acids for a number of applications, e.g., for preparation of peptides or other useful compounds.

In some embodiments, a compound (e.g., an amino acid or a protected and/or activated form thereof) or a salt thereof comprises 1) a first group which is an optionally protected amino group, 2) a second group which is an optionally protected and/or activated carboxyl group, and 3) a side chain (typically bonded to an atom between the first and second groups (“a side chain attachment atom”)) which comprises an optionally protected and/or activated carboxyl group and a) an optionally substituted ring (which ring is typically between the optionally protected and/or activated carboxyl group of the side chain and a side chain attachment atom) or b) an amino group (which amino group is typically between the optionally protected and/or activated carboxyl group of the side chain and a side chain attachment atom). In some embodiments, a provided compound is an optionally protected and/or activated amino acid or a salt thereof, wherein the side chain of the amino acid comprises an optionally protected and/or activated carboxyl group, and an optionally substituted ring or an amino group, wherein the optionally substituted ring or an amino group is between the optionally protected and/or activated carboxyl group and a backbone atom to which a side chain is attached (e.g., an atom between an amino and carboxyl group, both of which can be optionally and independently protected and/or activated (e.g., an alpha carbon atom in an amino acid)).

In some embodiments, the present disclosure provides compounds having the structure of formula PA:

or a salt thereof, wherein:
R^{PA} is —H or an amino protecting group;
each of R^{a1} and R^{a3} is independently -L^{a}-R′;
R^{a2} is -L^{aa}-C(O)R^{PS};
each of L^{a}, L^{a1} and L^{a2} is independently L;
—C(O)R^{PS} is optionally protected or activated —COOH;
—C(O)R^{PC} is optionally protected or activated —COOH;
each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms, C_{6 30} aryl, C_{6 30} arylaliphatic, C_{6 30} arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, an amino acid is described in WO 2022/261257, the amino acids of which are incorporated herein by reference.

As described above, each L is independently a covalent bond, or an optionally substituted, bivalent C₁-C₂₅ aliphatic or heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L is a covalent bond.

In some embodiments, L (or L^{a}, L^{aa}, L^{a1}, L^{a2}, L^{s1}, L^{s2}, L^{s3}, or another variable or moiety that can be L, or a linker moiety) is an optionally substituted, bivalent C₁-C₂₅, C₁-C₂₀, C₁-C₁₅, C₁-C₁₀, C₁-C₉, C₁-C₈, C₁-C₇, C₁-C₆, C₁-C₅, C₁-C₄, C₁-C₃, C₁-C₂, or C₁, C₂, C₃, C₄, C₅, C₆, C₇, C₈, C₉, C₁₀, C₁, C₁₂, C₁₃, C₁₄, C₁₅, C₁₆, C₁₇, C₁₈, C₁₉, or C₂₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent C₁-C₂₅, C₁-C₂₀, C₁-C₁₅, C₁-C₁₀, C₁-C₉, C₁-C₈, C₁-C₇, C₁-C₆, C₁-C₅, C₁-C₄, C₁-C₃, C₁-C₂, or C₁, C₂, C₃, C₄, C₅, C₆, C₇, C₈, C₉, C₁₀, C₁, C₁₂, C₁₃, C₁₄, C₁₅, C₁₆, C₁₇, C₁₈, C₁₉, or C₂₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₁-C₁₀, C₁-C₉, C₁-C₈, C₁-C₇, C₁-C₆, C₁-C₅, C₁-C₄, C₁-C₃, C₁-C₂, or C₁, C₂, C₃, C₄, C₅, C₆, C₇, C₈, C₉, or C₁₀, aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₂ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₃ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₄ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₅ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, it is an optionally substituted, bivalent C₆ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, the bivalent aliphatic is saturated. In some embodiments, the bivalent aliphatic is linear. In some embodiments, the bivalent aliphatic is branched. In some embodiments, it is an optionally substituted, bivalent linear saturated C₆ aliphatic wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each replacement if any is independently with —O—, —S—, —N(R′)—, —C(O)—, —S(O)—, —S(O)₂—, —S(O)₂N(R′)—, —C(O)S—, or —C(O)O—. In some embodiments, each replacement if any is independently with —O—, —S—, —N(R′)—, or —C(O)—. In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent C₁-C₆ linear saturated aliphatic wherein one or more methylene units is optionally and independently replaced with —O—, —S—, —N(R′)—, or —C(O)—. In some embodiments, it is an optionally substituted, bivalent C₁-C₅ linear saturated aliphatic wherein one or more methylene units is optionally and independently replaced with —O—, —S—, —N(R′)—, or —C(O)—. In some embodiments, it is an optionally substituted, bivalent C₁-C₄ linear saturated aliphatic wherein one or more methylene units is optionally and independently replaced with —O—, —S—, —N(R′)—, or —C(O)—. In some embodiments, it is an optionally substituted, bivalent C₁-C₃ linear saturated aliphatic wherein one or more methylene units is optionally and independently replaced with —O—, —S—, —N(R′)—, or —C(O)—. In some embodiments, it is an optionally substituted, bivalent C₁-C₂ linear saturated aliphatic wherein one or more methylene units is optionally and independently replaced with —O—, —S—, —N(R′)—, or —C(O)—. In some embodiments, it is a bivalent C₁-C₆ linear saturated aliphatic wherein one or more methylene units is optionally and independently replaced with —O—, —S—, —N(R′)—, or —C(O)—. In some embodiments, it is a bivalent C₁-C₅ linear saturated aliphatic wherein one or more methylene units is optionally and independently replaced with —O—, —S—, —N(R′)—, or —C(O)—. In some embodiments, it is a bivalent C₁-C₄ linear saturated aliphatic wherein one or more methylene units is optionally and independently replaced with —O—, —S—, —N(R′)—, or —C(O)—. In some embodiments, it is a bivalent C₁-C₃ linear saturated aliphatic wherein one or more methylene units is optionally and independently replaced with —O—, —S—, —N(R′)—, or —C(O)—. In some embodiments, it is a bivalent C₁-C₂ linear saturated aliphatic wherein one or more methylene units is optionally and independently replaced with —O—, —S—, —N(R′)—, or —C(O)—. In some embodiments, there is no replacement of methylene unit. In some embodiments, there is one replacement. In some embodiments, there is two replacement. In some embodiments, there is three replacement. In some embodiments, there is four or more replacement. In some embodiments, R′ in each moiety that is utilized to replace a methylene unit (e.g., —N(R′)—) as described herein is hydrogen or optionally substituted C₁₋₆ aliphatic or phenyl. In some embodiments, R′ is each such moiety is hydrogen or optionally substituted C₁₋₆ alkyl. In some embodiments, R′ is each such moiety is hydrogen or C₁₋₆ alkyl. In some embodiments, each -Cy- is optionally substituted bivalent ring selected from 3-10, 3-9, 3-8, 3-7, 5-10, 5-9, 5-8, 5-7, 5-6, or 3, 4, 5, 6, 7, 8, 9, or 10 membered cycloaliphatic and heterocyclylene having 1-3 heteroatoms, phenylene, and 5-6 membered heteroarylene having 1-3 heteroatoms. In some embodiments, -Cy- is optionally substituted bivalent 3-10, 3-9, 3-8, 3-7, 5-10, 5-9, 5-8, 5-7, 5-6, or 3, 4, 5, 6, 7, 8, 9, or 10 membered cycloaliphatic. In some embodiments, -Cy- is optionally substituted 3-10, 3-9, 3-8, 3-7, 5-10, 5-9, 5-8, 5-7, 5-6, or 3, 4, 5, 6, 7, 8, 9, or 10 membered heterocyclylene having 1-3 heteroatoms. In some embodiments, -Cy- is optionally substituted 3-10, 3-9, 3-8, 3-7, 5-10, 5-9, 5-8, 5-7, 5-6, or 3, 4, 5, 6, 7, 8, 9, or 10 membered heterocyclylene having 1 heteroatom. In some embodiments, -Cy- is optionally substituted phenylene. In some embodiments, -Cy- is phenylene. In some embodiments, -Cy- is optionally substituted 5-6 membered heteroarylene having 1-3 heteroatoms. In some embodiments, -Cy- is optionally substituted 5-6 membered heteroarylene having 1 heteroatom. In some embodiments, a heteroatom is nitrogen. In some embodiments, a heteroatom is oxygen. In some embodiments, a heteroatom is sulfur. In some embodiments, L, L^{a}, L^{aa}, L^{a1}, L^{a2}, L^{s1}, L^{s2}, L^{s3}, L″, or another variable or moiety that can be L, or a linker moiety, is optionally substituted —(CH₂)n-. In some embodiments, it is —(CH₂)n-. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, L, L^{a}, L^{aa}, L^{a1}, L^{a2}, L^{s1}, L^{s2}, L^{s3}, L″, or another variable or moiety that can be L, or a linker moiety, is an optionally substituted, bivalent heteroaliphatic group having 1-10 heteroatoms wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

Those skilled in the art appreciate that embodiments described for one linker moiety that can be L or L″ (e.g., L^{aa}, L^{s1}, L^{s2}, L^{s3}, L^{s}, L^{a}, L^{a1}, L^{a2}, L^{RN}, etc.) may also be utilized for another group that can be L or L″ to the extent that such embodiments fall within the definition of L or L″.

As described above, each R′ is independently -R, —C(O)R, —CO₂R, or —SO₂R. In some embodiments, R′ is -L^{a}-R. In some embodiments, R′ is R. In some embodiments, R′ is —C(O)R. In some embodiments, R′ is —CO₂R. In some embodiments, R′ is —SO₂R. In some embodiments, R′ is —H.

As described above, each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

As described herein, in some embodiments, R is —H. In some embodiments, R is not-H. In some embodiments, R is optionally substituted C₁₋₁₀ aliphatic. In some embodiments, R is optionally substituted C₁₋₁₀ alkyl. In some embodiments, R is methyl. In some embodiments, R is ethyl. In some embodiments, R is isopropyl. In some embodiments, R is —CF3. In some embodiments, R is —CH₂CF3. In some embodiments, R is butyl. In some embodiments, R is t-butyl. In some embodiments, R is optionally substituted C₃₋₁₀ cycloaliphatic. In some embodiments, R is optionally substituted C₃₋₁₀ cycloalkyl. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted phenyl. In some embodiments, R is phenyl. In some embodiments, R is optionally substituted 5-membered heteroaryl having 1-3 heteroatoms. In some embodiments, R is optionally substituted 5-membered heteroaryl having 1 heteroatom. In some embodiments, R is optionally substituted 6-membered heteroaryl having 1-3 heteroatoms. In some embodiments, R is optionally substituted 6-membered heteroaryl having 1 heteroatom. In some embodiments, R is optionally substituted bicyclic 8-10 membered aromatic ring having 0-5 heteroatoms. In some embodiments, R is optionally substituted bicyclic 9-membered aromatic ring having 1-5 heteroatoms. In some embodiments, R is optionally substituted bicyclic 10-membered aromatic ring having 1-5 heteroatoms. In some embodiments, R is optionally substituted bicyclic 9-membered aromatic ring having 1 heteroatom. In some embodiments, R is optionally substituted bicyclic 10-membered aromatic ring having 1 heteroatom. In some embodiments, R is optionally substituted bicyclic 10-membered aromatic ring having no heteroatom. In some embodiments, R is optionally substituted 3-10 membered heterocyclyl having 1-5 heteroatoms. In some embodiments, R is optionally substituted 5-14 membered bicyclic heterocyclyl having 1-5 heteroatoms.

In some embodiments, two R groups (or two groups that can be R, e.g., two groups each independently selected from R′, R^{a1}, R^{a2}, R^{a3}, R^{a5}, R^{RN}, etc.) are taken together with their intervening atom(s) to form an optionally substituted 3-30 membered, monocyclic, bicyclic or polycyclic ring having, in addition to the atom, 0-10 heteroatoms. In some embodiments, a formed ring is substituted. In some embodiments, a formed ring is unsubstituted. In some embodiments, a formed ring is 3-30, 3-20, 3-15, 3-10, 3-9, 3-8, 3-7, 3-6, 4-10, 4-9, 4-8, 4-7, 4-6, 5-10, 5-9, 5-8, 5-7, 5-6, or 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 membered. In some embodiments, a formed ring is 3-10 membered. In some embodiments, a formed ring is 3-7 membered. In some embodiments, a formed ring is 4-10 membered. In some embodiments, a formed ring is 4-7 membered. In some embodiments, a formed ring is 5-10 membered. In some embodiments, a formed ring is 5-7 membered. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is monocyclic. In some embodiments, a formed ring is bicyclic. In some embodiments, a formed ring is polycyclic. In some embodiments, a formed ring has no heteroatoms in addition to the intervening atom(s). In some embodiments, a formed ring has 1-10, e.g., 1-5, 1-3, or 1, 2, 3, 4, 5, 6, 7, 8, 9, or 10 heteroatoms in addition to the intervening atom(s). In some embodiments, a formed ring is saturated. In some embodiments, a formed ring is partially unsaturated. In some embodiments, a formed ring comprises one or more aromatic ring. In some embodiments, a formed ring is bicyclic or polycyclic, and each monocyclic unit is independently 3-10 membered, saturated, partially unsaturated or aromatic and having 0-5 heteroatoms. In some embodiments, each heteroatom is independently selected from nitrogen, oxygen and sulfur.

In some embodiments, a group that can be R, e.g., R′, R^{a1}, R^{a2}, R^{a3}, R^{a5}, R^{RN}, etc., is R as described herein. Those skilled in the art appreciate that embodiments described for one group that can be R may also be utilized for another group that can be R to the extent that such embodiments fall within the definition of R.

In some embodiments, as used in the present disclosure, e.g., in various embodiments or R, R′, various formulae, etc., each heteroatom is independently selected from oxygen, nitrogen, sulfur, phosphorus and silicon. In some embodiments, each heteroatom is independently selected from oxygen, nitrogen and sulfur. In some embodiments, in provided compound each heteroatom of heteroaryl, heterocyclyl, heteroaliphatic, ring (e.g., -Cy-, ring formed by groups taken together (e.g., two R groups taken together) with their intervening atom(s) (if any), etc.), etc., is independently selected from nitrogen, oxygen and sulfur. In some embodiments, in provided compound each heteroatom is independently selected from nitrogen, oxygen, sulfur, silicon and phosphorus. In some embodiments, rings (e.g., cycloaliphatic, aryl, heteroaryl, heterocyclyl, etc. in variables (e.g., R, R′ etc.), formulae, etc.) contain 1-30 (e.g., 2-30, 1-20, 2-20, 1-15, 2-15, 1-10, 2-10, 1-6, 2-6, etc.) ring carbon atoms and 0-15 (e.g., 0, 1-15, 0-10, 0-5, 1-10, 1-5, etc.) ring heteroatoms. In some embodiments, rings contain 1-20 ring carbon atoms and 0-10 ring heteroatoms. In some embodiments, rings contain 2-20 ring carbon atoms and 0-10 ring heteroatoms. In some embodiments, rings contain 1-10 ring carbon atoms and 0-5 ring heteroatoms. In some embodiments, rings contain 2-10 ring carbon atoms and 0-5 ring heteroatoms. In some embodiments, rings contain 2-10 ring carbon atoms and 0-5 ring heteroatoms independently selected from nitrogen, oxygen and sulfur. In some embodiments, rings formed by groups taken together (e.g., two R groups taken together) contain, in addition to the intervening atom(s) of such groups, 1-20 (e.g., 1-15, 1-10, 1-5, etc.) additional ring carbon atoms and 0-10 (e.g., 0, 1-10, 0-5, 1-5, etc.) additional ring heteroatoms. In some embodiments, such rings contain 1-15 additional ring carbon atoms and 0-5 ring heteroatoms. In some embodiments, such rings contain 1-10 additional ring carbon atoms and 0-5 ring heteroatoms. In some embodiments, each ring heteroatom is independently selected from nitrogen, oxygen and sulfur. As described herein, various groups may be optionally substituted. Substituents are routinely utilized in chemistry including in development of various therapeutics. Many substituents can be utilized in accordance with the present disclosure. In some embodiments, a substituent is a hydrocarbon group. In some embodiments, a substituent comprises a heteroatom. In some embodiments, a substituent comprises multiple heteroatoms. In some embodiments, each atom in a substituent is independently selected from hydrogen, carbon, halogen, nitrogen, oxygen, sulfur, phosphorus and silicon. In some embodiments, each atom in a substituent is independently selected from hydrogen, carbon, halogen, nitrogen, oxygen, and sulfur. In some embodiments, each atom in a substituent is independently selected from hydrogen, carbon, fluorine, chlorine, bromine, iodine, nitrogen, oxygen, and sulfur. In some embodiments, the total number of carbon and non-halogen heteroatom(s) in a substituent is about or no more than about 1; in some embodiments, it is no more than about 2; in some embodiments, it is no more than about 3; in some embodiments, it is no more than about 4; in some embodiments, it is no more than about 5; in some embodiments, it is no more than about 6; in some embodiments, it is no more than about 7; in some embodiments, it is no more than about 8; in some embodiments, it is no more than about 9; in some embodiments, it is no more than about 10; in some embodiments, it is no more than about 11; in some embodiments, it is no more than about 12; in some embodiments, it is no more than about 13; in some embodiments, it is no more than about 14; in some embodiments, it is no more than about 15; in some embodiments, it is no more than about 20. In some embodiments, the total number of carbon and non-halogen heteroatom(s) in each substituent is independently no more than about 20. In some embodiments, the total number of carbon and non-halogen heteroatom(s) in each substituent is independently no more than about 15. In some embodiments, the total number of carbon and non-halogen heteroatom(s) in each substituent is independently no more than about 10. In some embodiments, the total number of carbon and non-halogen heteroatom(s) in each substituent is independently no more than about 6.

Certain amino acids and structure moieties are described in WO 2022/020651, WO 2022/020652, and WO 2022/261257, the amino acids and structure moieties of each of which are independently incorporated herein by reference, and can be utilized in accordance with the present disclosure.

In some embodiments, an amino acid, or a structure moiety, of an amino acid or an agent (e.g., a peptide), is selected from below. A N-terminal cap (N-Term) is connected via R₁ to the amino group (R₁) of the first amino acid (AA1). In some embodiments, a N-Term cap may be properly considered as part of AA1. From there, each carboxylate (R₂) of that amino acid is connected to the amino group (R₁) of the subsequent amino acid, until the carboxylate (R₂) of the final amino acid is connected to R₁ of a C-terminal group. For any amino acid that has a branch point (R₃) and a branching monomer is indicated in brackets, R₁ of the monomer in brackets is attached to R₃ of the amino acid. Unless otherwise noted, for the amino acid Dap, with two potential branch points (R₃ and R₄), if two branches are indicated, the R₁ of the first branch is connected to R₃, and R₁ of the second branch connected to R₄; in some embodiments, only one branch is indicated and R₄ is —H. For any pair of amino acids that terminate in a *3 designation, the R₃ groups of each of those amino acids are linked to each other. Likewise, for any pair of amino acids that terminate in a **3 designation, the R₃ groups of those amino acids are linked to each other. For any sequence that contains a pair of branching amino acids with R₃ groups, and one contains a branching monomer that contains both R₁ and R₂ groups, then R₁ is attached to the branching amino acid adjacent to it in the sequence, and the R₂ group of the branching monomer is attached to R₃ of the amino acid with no branching monomer designated. For example, in various peptides that have one of Cys, hCys, Pen, or aMeC at position 10 and also one of Cys, hCys, Pen, or aMeC at position 14, and a branching group off of the amino acid residue 10, the R₁ of that branching group is tied to the R₃ of the amino acid residue at position 10, while the R₂ of that branching group is tied to the R₃ of the amino acid residue at position 14. For any amino acid which has a branching amino acid containing R₃ and nothing attached to it by the above, then R₃=H. Typically, all residues with terminal olefins are linked (stapled) by ring-closing metathesis. Certain examples are provided in Table E2 and Table E3. In some embodiments, the present disclosure provides agents, e.g., peptides such as stapled peptides, comprising one or more amino acid residues selected from below.

TABLE A-IV
Certain useful compounds or moieties.

| Compound/ | |
|---|---|
| Bracket Moiety | Structure |
| Ala | |
| Gly | |
| Cys | |
| His | |
| Asp | |
| Ile | |
| Glu | |
| Lys | |
| Phe | |
| Leu | |
| nLeu | |
| Arg | |
| Asn | |
| Ser | |
| Pro | |
| Thr | |
| Gln | |
| aThr | |
| GlnR | |
| Val | |
| Trp | |
| 4F3MeF | |
| Tyr | |
| Npg | |
| MePro | |
| Aib | |
| PL3 | |
| Cpg | |
| B5 | |
| Cbg | |
| PyrS2 | |
| CyLeu | |
| BztA | |
| Orn | |
| 3Thi | |
| Dab | |
| 2Thi | |
| TriAzLys | |
| 2F3MeF | |
| TriAzOrn | |
| sAla | |
| TfeGA | |
| sAbu | |
| iPrLys | |
| sCH2S | |
| MeAsn | |
| dLys | |
| hGlnR | |
| dOrn | |
| 2OH3COOHF | |
| DGlnR | |
| 4OH3COOHF | |
| DAsnR | |
| TriAzDap | |
| 3COOHF | |
| 4COOHF | |
| Hse | |
| 2COOHF | |
| Cha | |
| 5F3Me2COOHF | |
| 4F3Me2COOHF | |
| Cba | |
| 5F3Me3COOHF | |
| SbMeAsp | |
| 4F3MeCOOHF | |
| RbMeAsp | |
| 3F2COOHF | |
| 2FurA | |
| dGlu | |
| 2OMeF | |
| hTyr | |
| 2MeF | |
| 3cbmf | |
| 2BrF | |
| MorphNva | |
| 2ClF | |
| R4 | |
| 2CNF | |
| R5 | |
| 2NO2F | |
| R6 | |
| 2PyrA | |
| CypA | |
| 3PyrA | |
| Chg | |
| 4PryA | |
| Pff | |
| 3BrF | |
| DiethA | |
| 34MeF | |
| 4PipA | |
| 34ClF | |
| Abu | |
| Phg | |
| Nva | |
| DipA | |
| hLeu | |
| OctG | |
| Cpa | |
| F2PipNva | |
| MorphGln | |
| Aad | |
| 1NapA | |
| hPhc | |
| 2NapA | |
| hnLeu | |
| Me2Gln | |
| 2cbmf | |
| AcLys | |
| dOrn | |
| Met2O | |
| dDab | |
| Acp | |
| MeOrn | |
| 2Cpg | |
| Dap | |
| aMeL | |
| 4FF | |
| DaMeL | |
| 4ClF | |
| aMeV | |
| 4BrF | |
| aMeS | |
| 4CNF | |
| DaMeS | |
| 4MeF | |
| aMeF | |
| 3FF | |
| aMeDF | |
| 3ClF | |
| dAla | |
| 3BrF | |
| dLeu | |
| 3OMeF | |
| OAsp | |
| 3MeF | |
| Sar | |
| 3CNF | |
| NMebAla | |
| 2FF | |
| Aic | |
| [BzAm2OAllyl] | |
| RbiPrF | |
| [oXyl] | |
| SbiPrF | |
| [mXyl] | |
| RbiPrDF | |
| [pXyl] | |
| RbMeXylA | |
| [4FB] | |
| RbMeXylDA | |
| [8FBB] | |
| SbMeXylA | |
| [CH2CMe2CO2H] | |
| SbMeXylDA | |
| [NHEt] | |
| AzLys | |
| [29N2spiro- undecane] | |
| AllylGly | |
| [39N2spiro- undecane] | |
| [CyCO] | |
| [4mampiperidine] | |
| [Piv] | |
| [4aminopiperidine] | |
| [Phc] | |
| [diaminobutane] | |
| [Bn] | |
| NH2 | |
| [3butenyl] | |
| OH | |
| [Allyl] | |
| dAlaol | |
| [5hexenyl] | |
| Alaol | |
| [4pentenyl] | |
| Serol | |
| [3_3-biph] | |
| Prool | |
| [2_6-naph] | |
| Throl | |
| 4TriA | |
| [2COOH4NH2Ph] | |
| 3F3MeF | |
| [2COOH4NO2Ph] | |
| AsnR | |
| [2COOHPh] | |
| aMeDAsp | |
| [2Nic] | |
| [isophthalate] | |
| [2OxoPpz] | |
| [succinate] | |
| [3C] | |
| [Me2MaI] | |
| [3Py] | |
| [diphenate] | |
| [4AcMePip | |
| [Biphen33COOH] | |
| [4CF3PhAc | |
| ThioPro | |
| [4F3CPip] | |
| [4MePpzPip] | |
| [EtSSEt] | |
| ]4Pippip] | |
| [EtSSHex] | |
| ]4PyPip] | |
| [EtSSPh] | |
| [Ac] | |
| [EtSSpy] | |
| [AcPpz] | |
| [H4IAP] | |
| [bismethoxy- ethylamine] | |
| [isoindoline] | |
| [Bn] | |
| [lithocholate] | |
| [CCpCO2H] | |
| [PEG2] | |
| [CF3CO] | |
| [Me] | R₁— |
| [CH2CChCO2H] | |
| [Me2diaminobutane] | |
| [CH2CCpCO2H] | |
| [Me2NCBz] | |
| [CH2CH2CO2H] | |
| [Me2Npr] | |
| [CH2CMe2CO2H] | |
| [Me2NPrPip] | |
| [CH2CO2H] | |
| [Me3AdamantC] | |
| [CH2NMe2] | |
| [MeMorphBz] | |
| [CH2Ppz] | |
| [MePipAc] | |
| [CMe2CO2H] | |
| [MeSO2] | |
| [CyPr] | |
| [Morph] | |
| [Et] | |
| [MorphAc] | |
| [EtSO2Ppz] | |
| [MorphCH2] | |
| [MorphEt] | |
| 2F3MeW | |
| [NdiMeButC] | |
| 2NH2F | |
| [NHBn] | |
| 34ClF | |
| [NHEt] | |
| 34MeF | |
| [NMe2] | |
| 3Br4FF | |
| [PfbGA] | |
| 3BrF | |
| [Pfbn] | |
| [3CBMF] | |
| [PfBz] | |
| 3CH2NMe2F | |
| [PfPhAc] | |
| 3CO2PhF | |
| [Ph] | |
| 3SF | |
| [Phc] | |
| 3SO2F | |
| [Pic] | |
| 3TzF | |
| [Ppz] | |
| 4BrF | |
| [RDMAPyr] | |
| 4ClBztA | |
| [Red] | |
| 4ClW | |
| [sBu] | |
| 4F3COOHF | |
| [SO2MorphCH2] | |
| 4FW | |
| [Tfb] | |
| 4SEF | |
| [TfePpz] | |
| 4TzF | |
| [Tfp] | |
| 5F3MeCOOHF | |
| 5IndA | |
| BzAm3Oallyl | |
| 5iPr3COOHF | |
| Cba | |
| 7AzaW | |
| Cbg | |
| 7ClBztA | |
| ClAc | |
| 7FBztA | |
| CO | |
| AcAsp | |
| CO2Bu | |
| AcLys | |
| CO2Hex | |
| AspE | |
| CO2iBu | |
| AspSH | |
| CO2Me | |
| Az2 | |
| CO2Ph | |
| Az3 | |
| Cpg | |
| B3 | |
| CyLeu | |
| B4 | |
| dAla | |
| B6 | |
| dIle | |
| bMe2Asp | |
| dLeu | |
| Bn3OAllyl | |
| F2PipAbu | |
| BnBoroleK | |
| F2PipNva | |
| Bnc | |
| GA | |
| BrAc | |
| GAbu | |
| BzAm2Allyl | |
| GlnR | |
| GluE | |
| NMebAla | |
| GluSH | |
| Npa | |
| hhLeu | |
| PAc3OAllyl | |
| HypBzEs3OAllyl | |
| ProAm5 | |
| HypEs4 | |
| ProAm6 | |
| HypEs5 | |
| ProBzAm3OAllyl | |
| HypPAc3OAllyl | |
| ProPAc3OAllyl | |
| Me2Asn | |
| PropynOH | |
| Me2Gln | |
| ProSAm3 | |
| MeAsn | |
| PyrR | |
| MeGln | |
| PyrR2 | |
| MePpzAbu | |
| PyrS4 | |
| MePpzAsn | |
| R2COOPipA | |
| MePpzNva | |
| R3COOPipA | |
| MePro | |
| RbMe2NapA | |
| Met2O | |
| RbMeBztA | |
| MorphAbu | |
| RbOHAsp | |
| MorphAsn | |
| S2COOPipA | |
| MorphGlN | |
| S3COOPipA | |
| MorphNva | |
| sAc | |
| sAla | |
| SPip2 | |
| Sar | |
| Spip3 | |
| SbMe2NapA | |
| sPr | |
| SbMeBztA | |
| TriAzDap | |
| sBut | |
| TriAzDab | |
| SeNc5 | |
| TriAzLys | |
| Spip1 | |
| TriAzdLys | |
| BiotinPEG8 | |

Table A-IV (Continued; certain moieties may be presented in [ ])
Certain moieties useful as, e.g. Lys analogs, branch point amino acid residues, or non-RCM stapling amino acid residues

| | |
|---|---|
| sAla | sAbu |
| sCH2S | dLys |
| dOm | DGlnR |
| DAsnR | dOrn |
| dDab | iPrLys |
| TriAzDap | 4PipA |
| GlnR | AsnR |
| NMeOrn | Dap |
| hGlnR | Orn |
| Dab | TriAzLys |
| TriAzOrn | Acp |
| AmPhe | 4AmPhe |
| Pen | 1MeK |
| hGlnR | AsnEDA |
| GlnEDA | AsnPpz |
| GlnPpz | GlnR3APyr |
| GlnS3APyr | GlnMe2EDA |
| AsnMe2EDA | AsnMeEDA |
| AsnR3APyr | AsnS3APyr |
| PyrRA | PyrSa |

hCysOx
Certain moieties useful as, e.g., stapling amino acid residues (e.g., RCM for other stapling technologies)

| | |
|---|---|
| PL3 | B5 |
| PyrS2 | PyrS3 |
| PyrS | PyrS1 |
| R4 | R5 |
| R6 | R7 |
| R8 | B4 |
| B3 | B6 |
| RgN | SgN |
| RdN | SdN |
| ReN | SeN |
| S4 | S5 |
| S6 | S7 |
| S8 | Az |
| SeNc5 | AllylGly |
| PyrS4 | PyrS5 |
| PyrR2 | PyrR |
| PyrSc72RMe3SOMe | Az2 |
| Az3 | SPip2 |
| SPip3 | PyrSc72SMe3ROMe |
| PyrSc7 | PyrSc73Me2 |
| DapAc7 | PyrSc7O45RMe |
| PyrSc7O45SMe | PyrSaA3Butene |
| PyrSadA3Butene | Dap7Gly |
| Dap7Pent | DapAc7EDA |
| DapAc7PDA | Dap7Abu |
| 4VinylPyrSa | 3allyloxyPyrSa |
| PyrSaV3Butene | PyrSadV3Butene |
| PyrSaSar3Butene | PyrSaNip3Butene |
| PyrSadNip3Butene | PyrSaPro3Butene |
| PyrSa4VinMe2PhAc | 3allylPyrSa |

Certain moieties useful as, e.g., aromatic amino acid residues

| | |
|---|---|
| 4F3MeF | BztA |
| 3Thi | 2Thi |
| 2F3MeF | 3COOHF |
| 2OH3COOHF | 4OH3COOHF |
| Pff | 3cbmf |
| 4COOHF | 2COOHF |
| 4F3Me2COOHF | 5F3MeCOOHF |
| 4F3Me3COOHF | 3F2COOHF |
| 5F3Me2COOHF | 2FurA |
| 2OMeF | 2MeF |
| 2BrF | 2ClF |
| 2CNF | 2NO2F |
| 2PyrA | 3PyrA |
| 4PyrA | 3BrF |
| 34MeF | 34ClF |
| Phg | DipA |
| 2cbmf | 1NapA |
| 2NapA (2Nal) | 4FF |
| 4ClF | 4BrF |
| 4CNF | 4MeF |
| 3FF | 3ClF |
| 3BrF | 3OMeF |
| 3MeF | 3CNF |
| 2FF | Aic |
| RbiPrF | SbiPrF |
| RbiPrDF | RbMeXylA |
| RbMeXylDA | SbMeXylA |
| SbMeXylDA | 4cbmf |
| 6F1NapA | Qui |
| 5MeOW | 345FF |
| 34FF | WCHO |
| 3F3MeF | 4F3MeF |
| 245FF | 1MeH |
| tetz | 3MeH |
| 4Thz | 5ClW |
| 6ClW | 1MeW |
| 6FW | 6ClaMeW |
| 5ClW | d1NapA |
| d2NapA | CF3Tyr |
| 34OMeF | 24MeF |
| h4PyrA | 6CNW |
| 6MeW | 6FW |
| 3FTyr | 35BrY |
| 3ClY | 3NO2Y |
| OTrp | c6Phe |
| 3BOH2F | 4BOH2F |
| 7FBztA | 7ClBztA |

7MeBztA
Certain moieties useful as amino acid residues

| | |
|---|---|
| Ala | Cys |
| Asp | Glu |
| Phe | Gly |
| His | Ile |
| Lys | Leu |
| Met | Asn |
| Pro | Gln |
| Arg | Ser |
| Thr | Val |
| Trp | Tyr |

Certain moieties useful as, e.g., amino acid residues (e.g., D-amino acid residues, homologated amino acid
residues, alkyl (e.g., methyl) amino acid residues, etc.)

| | |
|---|---|
| MePro | dGlu |
| hTyr | hLeu |
| aMeL | DaMeL |
| hPhe | Sar |
| dPro | aMeV |
| aMeS | DaMeS |
| aMeF | aMeDF |
| dAla | dLeu |
| aMeDAsp | NMeS |
| aMeW | dPhe |
| SDMA | ADMA |
| hArg | hhSer |
| hhLeu | hCys |

aMeC
Certain moieties useful as, e.g., amino acid residues (e.g., alkyl amino acid residues, hydrophobic amino acid residues, etc.)

| | |
|---|---|
| Npg | Aib |
| Cpg | Cbg |
| CyLeu | Cha |
| nLeu | Chg |
| Tle | NMebAla |
| OctG | DiethA |
| Cba | Abu |
| Nva | Cpa |
| 2Cpg | hnLeu |
| CypA | Abu |
| alle | F3CA |
| HF2CA | Ppg |
| hCba | hCypA |
| hCha | Nip |
| dNip | 4Abu |

NHPent
Certain moieties useful as, e.g., amino acid residues (e.g., polar amino acid residues, basic amino acid residues, etc.)

| | |
|---|---|
| aThr | Hse |
| Me2Gln | Met2O |
| MeAsn | S(OMe) |
| AcLys | MorphGln |
| TOMe | MorphNva |
| AzLys | F2PipNva |

Cit
Certain moieties useful as, e.g., amino acid residues (e.g., acidic amino acid residues, non-aromatic amino acid residues, etc.)

| | |
|---|---|
| isoDAsp | RbGlu |
| SbGlu | SbMeAsp |
| RbMeAsp | Aad |
| TfeGA | OAsp |
| EtGA | PyrSaa |
| bMe2Asp | [Malonate]Dap |
| [Me2Mal]Dap | [RaiPrSuc]Dap |
| [SaiPrSuc]Dap | [SaMeSuc]Dap |

[Succinate]Dap
Certain moieties (e.g., moieties utilized in [ ] in various agents)

| | |
|---|---|
| CyCO | Piv |
| Phc | Bn |
| 5hexenyl | 4pentenyl |
| 3_3-biph | 2_6-naph |
| BzAm2OAllyl | oXyl |
| mXyl | pXyl |
| 4FB | 8FBB |
| CH2CMe2CO2H | NHEt |
| 29N2spiroundecane | 39N2spiroundecane |
| 4mampiperidine | 4aminopiperidine |
| diaminobutane | NH2 |
| OH | dAlaol |
| Alaol | Serol |
| Prool | Throl |
| isophthalate | Tf |
| 35CF3PhPr | 1NapPr |

BiotinPEG8

| | |
|---|---|
| succinate | Me2Mal |
| diphenate | Biphen33COOH |
| Bip | Acryl |
| 22PhPr | F3CCro |
| MeSO2 | NHiPr |
| NHnPr | NHCyPr |
| NHCyBu | NHMe |
| MorphAc | MePipAc |
| Ac | CIAc |
| IAc | MeBipipAc |
| 4MePipBz | MeMorphBz |
| Me2NCBz | mPEG2 |

```
  mPEG4
  mPEG6
  mPEG8
```

| | |
|---|---|
| Bua | Hex |
| C2Mal | Oct |
| AdamantC | Me3AdamantC |
| AdamantPro | 6AmHex |
| Leuol | Pheol |
| C3Mal | ES |
| 3MPA | 5Pnyl |
| isovaleryl | 2PyBu |
| 1lmidac | 2F2PyAc |
| 2lAPAc | 124TriPr |
| 6QuiAc | 3PyAc |
| 123TriAc | 1PyrazoleAc |
| 4PyPrpc | 3PyPrpc |
| 5PymAc | 1PydoneAc |
| 124TriAc | 3IAPAc |
| Me2NAc | 4MePipzPrpC |
| Melmid4SO2 | 8QuiSO2 |
| PfBn | Tfb |
| mPyr | C3 |
| mPEG16 | mPEG24 |
| mPEG37 | CH2CO2H |
| SaiPrSuc | RaiPrSuc |
| SaMeSuc | Red |
| 33Oxe | IsoE |
| 13Ac | m5Meb |
| m5Pyr | m5OMeb |

Certain moieties (e.g., moieties utilized in [ ] in various agents, amino acid residues, etc.)

| | |
|---|---|
| NPyroR3 | C3a |
| isobutyryl | Cpc |
| Cbc | CypCO |
| 2PyCypCO | 4THPCO |
| 2NH2F | Bnc |

```
  BiPh
  PEG4triPEG16
  PEG4triPEG36
```

| | |
|---|---|
| diaminopentane | diaminopropane |
| diaminobutane | malonate |
| Me2diaminohexane | Me2ethylenediamine |
| 15PyraPy | 2FPhc |
| 3Phc3 | 4PymCO |
| 5PymCO | 8IAP |
| MeOPr | Ts |
| ethylenediamine | 2PyzCO |
| 2PhF | Ala_D3 |
| CyhLeu | Dpg |
| HepG | HexG |
| NAsp | NGlu |
| 2AmMePhe | 3AmMePhe |
| 4AmMePhe | 24FF |
| 46ClW | 4CNW |
| 5MeW | 5Pnyl |
| 6FlW | 7ClW |
| 7MeW | PyrR3 |
| 2IAPAc | 6QuiAc |
| 2PyBu | 34FF |
| Az1 | Az |
| 46ClW | CF3CO |
| 6QuiAc | 124TriAc |
| 5PymAc | 2PyCypCO |
| 2PyzCO | 124TriPr |
| 2PyAc | 2PyPrpc |
| 3PyPrpc | 4PyPrpc |
| PhOPr | Me2MeOPr |
| Me2NPr | NdiMeButC |
| 3IAPAc | 15PyraPy |
| MorphAc | Nic |
| 5PymCO | 1MeK |
| dPEG2 | Oct |

```
  PEG8
  C18
```

| | |
|---|---|
| GlnPDA | GlnBDA |
| GlnMePDA | GlnMe2BDA |
| GlnMeBDA | Gln5DA |
| Gln6DA | GlnT4CyMe |
| AcLys | GlnC4CyMe |
| Gln3ACPip | GlnPipAz |
| lithocholate | Gln4Pippip |
| GlnPip4AE | S3MePyrSc7 |
| R3MePyrSc7 | S3iPrPyrSc7 |
| R3iPrPyrSc7 | 3FPyr2c |
| 5ClW | 6ClW |
| 4FPyr3c | 2PymCO |
| 3PydCO | TzPyr |

In some embodiments, within a bracket there are two moieties, e.g., [Ac-dPEG2], typically R₁ of the first is connected to R₁ of the latter. For example, in [Ac-dPEG2], R₁ of Ac is connected to R₁ of dPEG2. R₂ of dPEG2 can be connected to other moieties, e.g., in [Ac-dPEG2]-Lys, R₃ of Lys.

In some embodiments, the present disclosure provides an agent, e.g., a peptide agent (in various embodiments, a stapled peptide agent), comprising a moiety selected from the table above. In some embodiments, a residue is stapled, e.g., forming a staple with another moiety. In some embodiments, an agent comprises a staple formed between two moieties each independently selected from the table above. In some embodiments, a staple comprises a double bond. In some embodiments, a staple comprises an E double bond. In some embodiments, a staple comprises a Z double bond. In some embodiments, a double bond is converted into another moiety, e.g., to a saturated bond through hydrogenation, an epoxide through epoxidation, etc. In some embodiments, a moiety, e.g., an amino acid residue, comprises two groups that can be utilized for stapling. In some embodiments, an amino acid residue comprises two groups for stapling, e.g., B3, B4, B5, B6, Dap7Gly, Dap7Pent, DapAc7EDA, DapAc7PDA, Dap7Abu, etc. In some embodiments, a N-terminal group, e.g., 4pentenyl, 5hexenyl, etc., may be considered as part of the first amino acid residue for stapling. In some embodiments, amino acid residues with N-terminal groups (e.g., 4pentenyl, 5hexenyl, etc.) such as 4pentenyl-PL3, 5hexenyl-PL3, etc., comprise two groups, e.g., two double bonds, for stapling. In some embodiments, a group for stapling is a double bond. In some embodiments, each group for stapling is independently a double bond. In some embodiments, a group for stapling is a double bond and the other is not (e.g., amino group, or a group which is or comprises R₃). In some embodiments, an agent comprise one and no more than one residue comprising two or more residues for stapling. In some embodiments, an agent comprising one and no more than one amino acid residue that is bonded to two staples. In some embodiments, agents comprise staples having different types of structures and/or formed by different types of transformations. For example, in some embodiments, an agent comprises a staple whose formation does not comprises an olefin metathesis transformation and/or modification of a carbon-carbon double bond (e.g., hydrogenation). In some embodiments, such agents may provide improved properties, activities, design flexibility, manufacturing efficiency, etc.

In some embodiments, a compound has a structure selected from the table above, wherein R₁ is —OH. In some embodiments, a compound has a structure selected from the table above, wherein R₁ is —H. In some embodiments, a compound is a compound has the structure selected from the table above, wherein R₁ is —H or amino protecting group (e.g., Fmoc, tBoc, etc.) and R₂ is —OH, a carboxyl protecting or activating group, or a salt thereof. In some embodiments, a compound is a compound has the structure selected from the table above, wherein R₁ is —H or amino protecting group and R₂ is —OH, or a salt thereof. In some embodiments, a compound is a compound has the structure selected from the table above, wherein R₁ is —H and R₂ is —OH, or a salt thereof. In some embodiments, a compound is a compound has the structure selected from the table above, wherein R₁ is —H, R₂ is —OH and R₃ is —H, or a salt thereof. In some embodiments, R₃ is —H or a protecting group. In some embodiments, R₃ is —H. In some embodiments, a compound has a structure selected from the table above, wherein R₁ is an amino protection group, e.g., Fmoc, tBoc, etc. In some embodiments, a compound has a structure selected from the table above, wherein R₁ is an amino protecting group, e.g., Fmoc, tBoc, etc., and R₂ is —OH, or —COR₂ is an optionally substituted, protected or activated carboxyl group. In some embodiments, R₂ is —OH. In some embodiments, an amino acid residue has a structure selected from the table above, wherein each of R₁ and R₂ independently represents a connection site (e.g., for structure

the residue is of the structure

In some embodiments, an agent, a peptide or a stapled peptide comprises such an amino acid residue.

Among other things, the present disclosure provides peptides, including stapled peptides, comprising residues of amino acids described herein. In some embodiments, the present disclosure provides various methods comprising utilizing amino acids, optionally protected and/or activated, as described herein. In some embodiments, the present disclosure provides methods for preparing peptides, comprising utilizing amino acids, typically protected and/or activated, as described herein. For example, in some embodiments, various amino groups are Fmoc protected for peptide synthesis (particularly for forming backbone peptide bonds). In some embodiments, various side chain carboxylic acid groups are t-Bu protected (—C(O)—O-tBu).

In some embodiments, an agent, e.g., a peptide, a stapled peptide, a stitched peptide, etc., is less than about 5000 Daltons in mass. In some embodiments, an agent is greater than or equal to about 900 Daltons and less than about 5000 Daltons in mass. In some embodiments, an agent is greater than or equal to about 1500 Daltons and less than about 5000 Daltons in mass. In some embodiments, an agent is greater than or equal to about 2000 Daltons and less than about 5000 Daltons in mass. In some embodiments, an agent is greater than or equal to about 2500 Daltons and less than about 5000 Daltons in mass. In some embodiments, an agent is greater than or equal to about 1000 Daltons and less than about 3000 Daltons in mass. In some embodiments, an agent is greater than or equal to about 1500 Daltons and less than about 3000 Daltons in mass. In some embodiments, an agent is greater than or equal to about 1500 Daltons and less than about 2500 Daltons in mass. In some embodiments, an agent is greater than or equal to about 1600 Daltons and less than about 2200 Daltons in mass. In some embodiments, the agent is no more than about 900 Daltons in mass. In some embodiments, an agent is no more than about 500 Daltons in mass. In some embodiments, an agent is no more than about 300 Daltons in mass. In some embodiments, an agent is no more than about 200 Daltons in mass.

### Characterization

In some embodiments, agents, e.g., peptides, are characterized with respect to, for example, one or more characteristics such as binding characteristics—e.g., with respect to a particular target of interest (e.g., beta-catenin or a portion thereof), stability characteristics, for example in solution or in dried form, cell permeability characteristics, solubility, lipophilicity, etc.

In some embodiments, a binding characteristic may be or comprise specificity, affinity, on-rate, off-rate, etc, optionally under (or over a range of) specified conditions such as, for example, concentration, temperature, pH, cell type, presence or level of a particular competitor, etc.

As will be appreciated by those skilled in the art, assessments of characteristics as described herein may involve comparison with an appropriate reference (e.g., a positive or negative control) which may, in some embodiments, be a contemporaneous reference or, in some embodiments, a historical reference.

In some embodiments, desirable characteristics may be, for example: binding to a desired target (e.g., a dissociation constant (K_{D}) of at least less than about 1 uM, and preferably a K_{D} of less than about 50 nM); cell penetration (e.g., as measured by fluorescence-based assays or mass spectrometry of cellular fractions, etc.); solubility (e.g., soluble at less than about 1000 uM agent, or soluble at less than about 500 uM agent, or soluble at less than about 100 uM agent, or less than about 50 uM, or less than about 35 uM); activity (e.g., modulating one or more functions of a target, which may be assessed in a cellular reporter assay (e.g., with an IC50 of less than a concentration, e.g., less than about 1 μM, less than about 500 nM, less than about 50 nM, less than about 10 nM, etc.), an animal model (e.g., various animal models described in the Examples) and/or a subject; stability, which may be assessed using a number of assays (e.g., in a rat pharmacokinetic study (e.g., administered via oral, iv, ip, etc.) with a terminal half-life of greater than a suitable time, e.g., 1 hour); low toxicity, which might be assessed by a number of assays (e.g., a standard ADME/toxicity assays); and/or low levels of cytotoxicity (e.g., low levels of lactate dehydrogenase (LDH) released from cells when treated at a suitable concentration, e.g., about 10 UM of a peptide). In some embodiments, an agent of the invention comprises an affinity of less than about 10 nM, for example, an IC50 of 7 nM).

In some embodiments, provided agents can bind to targets, e.g., beta-catenin, with an EC 50 of no more than about 2000 nM. In some embodiments, an EC50 is no more than about 1500 nM. In some embodiments, an EC50 is no more than about 1000 nM. In some embodiments, an EC50 is no more than about 500 nM. In some embodiments, an EC50 is no more than about 300 nM. In some embodiments, an EC50 is no more than about 200 nM. In some embodiments, an EC50 is no more than about 100 nM. In some embodiments, an EC50 is no more than about 75 nM. In some embodiments, an EC50 is no more than about 50 nM. In some embodiments, an EC50 is no more than about 25 nM. In some embodiments, an EC50 is no more than about 10 nM. In some embodiments, an EC50 is no more than about 5 nM. In some embodiments, an EC50 is measured by fluorescence polarization as described in the Examples.

In some embodiments, the present disclosure provides agents, e.g., stapled peptides, with suitable solubility for various purposes. In some embodiments, solubility of provided agents, e.g., in PBS, is about or at least about 5-100 uM (e.g., about or at least about 5, 10, 15, 20, 25, 30, 40, 50, 60, 70, 80, 90, or 100 uM). In some embodiments, solubility is about or at least about 25 uM. In some embodiments, solubility is about or at least about 30 uM. In some embodiments, solubility is about or at least about 40 uM. In some embodiments, solubility is about or at least about 50 uM. In some embodiments, provided agents, e.g., stapled peptides, are protein bound in serum; in some embodiments, they are at least about 85%, 90%, or 95% protein bound in serum. In some embodiments, provided agents are over 95% protein bound in scrum.

In some embodiments, provided agents can traverse a cell membrane of an animal cell. In some embodiments, provided agents can traverse a cell membrane of a human cell.

Among other things, provided agents can bind to motifs, residues, or polypeptides. In some embodiments, provided agents bind to beta-catenin. In some embodiments, a dissociation constant (K_{D}) is about 1 nM to about 1 uM. In some embodiments, a K_{D} is no more than about 1 uM. In some embodiments, a K_{D} is no more than about 500 nM. In some embodiments, a K_{D} is no more than about 250 nM. In some embodiments, a K_{D} is no more than about 100 nM. In some embodiments, a K_{D} is no more than about 50 nM. In some embodiments, a K_{D} is no more than about 25 nM. In some embodiments, a K_{D} is no more than about 10 nM. In some embodiments, a K_{D} is no more than about 5 nM. In some embodiments, a K_{D} is no more than about 1 nM. As appreciated by those skilled in the art, various technologies are available and can be utilized to measure K_{D} in accordance with the present disclosure. In some embodiments, K_{D} is measured by Surface Plasmon Resonance (SPR) as illustrated herein.

In some embodiments, provided agents binds to a polypeptide whose sequence is or comprising SEQ ID NO: 2, or a fragment thereof:

(SEQ ID NO: 2)
SVLFYAITTLHNLLLHQEGAKMAVRLAGGLQKMVALLNKTNVKFL
AITTDCLQILAYGNQESKLIILASGGPQALVNIMRTYTYEKLLWT
TSRVLKVLSVCSSNKPAIVEAGGMQALGLHLTDPSQRLVQNCLWT
LRNLSDAATKQEGMEGLLGTLVQLLGSDDINVVTCAAGILSNLTC
NNYKNKMMVCQVGGIEALVRT.

In some embodiments, provided agents have one or more or all of the following interactions with beta-catenin:

Direct interactions (), water mediated ||,
non-polar contacts {}
(SEQ ID NO: 3)
LQIL{AY}(G){NQ}ES(K)LIILA
(residue 301-317 of Uniprot P35222 sequence)
(SEQ ID NO: 4)
SRVL{(K)V}LS{V]CSSN
(residue 341-353 of Uniprot P35222 sequence)
(SEQ ID NO: 5)
RLV{QN}C[L](W)TL{R}(N)LSDA
(residue 376-391 of Uniprot P35222 sequence)
(SEQ ID NO: 6)
LGSD[D]I(N){V}V{TC}AAGI
(residue 409-423 of Uniprot P35222 sequence)

In some embodiments, an agent, e.g., a peptide, binds to beta-catenin and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions: A305, Y306, G307, N308, Q309, K312, K345, V346, V349, Q379, N380, L382, W383, R386, N387, D413, N415, V416, T418, and C₄₁₉. In some embodiments, an agent, e.g., a peptide, binds to beta-catenin and interacts with one or more residues that are or correspond to at least two, or at least three, or at least four, or at least five, or at least six, or seven of the following amino acid residues in SEQ ID NO: 1 at the indicated positions: G307, K312, K345, W383, R386, N387, D413, and N415. In some embodiments, an agent, e.g., a peptide, binds to beta-catenin and interacts with one or more residues that are or correspond to at least two, or at least three, or at least four, or at least five, or at least six, or seven of the following amino acid residues in SEQ ID NO: 1 at the indicated positions: G307, K312, K345, W383, N387, D413, and N415.

In some embodiments, provided agents interact with beta-catenin at one or more (e.g., 1, 2, 3, 4, 5, 6, 7, 8 or 9) of G307, K312, K345, Q379, L382, W383, N387, N415 and V416. In some embodiments, provided agents interact with beta-catenin at one or more (e.g., 1, 2, 3, 4, 5, 6, 7, 8, 9 or 10) of Y306, G307, K312, K345, Q379, L382, W383, N387, N415 and V416. In some embodiments, provided agents interact with beta-catenin at one or more (e.g., 1, 2, 3, 4, 5, 6, 7, 8, 9 or 10) of G307, K312, K345, Q379, L382, W383, R386, N387, N415 and V416. In some embodiments, provided agents interact with beta-catenin at one or more (e.g., 1, 2, 3, 4, 5, 6, 7, 8, 9, 10 or 11) of Y306, G307, K312, K345, Q379, L382, W383, R386, N387, N415 and V416. In some embodiments, provided agents interact with beta-catenin at one or more (e.g., 1, 2, 3, 4, 5, 6, 7, 8, 9, 10, 11 or 12) of Y306, G307, K312, K345, V349, Q379, L382, W383, R386, N387, N415 and V416. In some embodiments, provided agents interact with beta-catenin at one or more (e.g., 1, 2, 3, 4, 5, 6, or 7) of G307, K312, K345, W383, R386, N387, D413 and N415. In some embodiments, provided agents interact with beta-catenin at one or more (e.g., 1, 2, 3, 4, 5, 6, or 7) of G307, K312, K345, W383, N387, D413 and N415. In some embodiments, provided agents interact with beta-catenin at one or both of K312 and R386. In some embodiments, provided agents interact with G307. In some embodiments, provided agents interact with K312. In some embodiments, provided agents interact with beta-catenin at one or more of K345, W383, D413 and N415. In some embodiments, provided agents interact with beta-catenin at one or more of K345 and W383. In some embodiments, provided agents interact with beta-catenin at one or more of D413 and N415. In some embodiments, provided agents interact with Y306. In some embodiments, provided agents interact with G307. In some embodiments, provided agents interact with K312. In some embodiments, provided agents interact with K345. In some embodiments, provided agents interact with V349. In some embodiments, provided agents interact with Q379. In some embodiments, provided agents interact with L382. In some embodiments, provided agents interact with W383. In some embodiments, provided agents interact with R386. In some embodiments, provided agents interact with N387. In some embodiments, provided agents interact with D413. In some embodiments, provided agents interact with N415. In some embodiments, provided agents interact with V416.

In some embodiments, provided agents interact with one or more of amino acid residues that are or correspond to K312, R386, K345 and W383 of SEQ ID NO: 1. In some embodiments, provided agents interact with one or more of amino acid residues that are or correspond to K312 and R386 of SEQ ID NO: 1. In some embodiments, interaction with an amino acid residue can be assessed through mutation of such an amino acid residue (e.g., mutation of K, R, etc. to D, E, etc.).

As those skilled in the art reading the present disclosure will appreciate, in some embodiments, interactions with beta-catenin may be assessed by contacting an agent with either a full-length or a portion of beta-catenin. In some embodiments, a portion of beta-catenin comprises the interacting residues above. In some embodiments, a portion of beta-catenin is or comprises SEQ ID NO: 2. In some embodiments, a portion of beta-catenin is expressed with a tag (e.g., for purification, detection, etc.). In some embodiments, a tag is a fluorescent tag. In some embodiments, a tag is for detection. In some embodiments, a tag is for purification and detection. In some embodiments, a tag is a purification tag. In some embodiments, a tag is or comprises biotin. Many other types of tags are available in the art and can be utilized in accordance with the present disclosure.

Various technologies can be utilized for characterizing and/or assessing provided technologies (e.g., agents (e.g., various peptides), compositions, methods, etc.) in accordance with the present disclosure. As described herein, in some embodiments, a useful technology is or comprises fluorescence polarization. In some embodiments, a useful technology assesses LogP or LogD. In some embodiments, a useful technology is or comprises a CHI LogD assay. In some embodiments, a useful technology assesses solubility. In some embodiments, a useful technology is or comprises NanoBRET. In some embodiments, a useful technology is or comprises a reporter assay (e.g., DLD1 reporter assay). In some embodiments, a useful technology is or comprises alphascreen. Certain useful protocols are described in the Examples. Those skilled in the art appreciate that suitable adjustments may be made to such protocols, e.g., according to specific conditions, agents, purposes, etc.

In some embodiments, tumor models are utilized to assess technologies. In some embodiments, a tumor model is a patient-derived tumor model. In some embodiments, a number of tumor models are utilized. In some embodiments, a number of patient-derived tumor models are utilized. In some embodiments, tumor models carry various mutations. Certain mutations are described in the Examples.

### Production

Various technologies are known in the art for producing provided agents. For example, various technologies for preparing small molecules, peptides (including stapled peptides) may be utilized in accordance with the present disclosure. Those skilled in the art, reading the present disclosure will well appreciate which such technologies are applicable in which aspects of the present disclosure in accordance with the present disclosure.

Stapling may be performed during and/or after peptide chain synthesis. In some embodiments, the present disclosure provides an unstapled peptide agent whose sequence is one described in Table E2 or Table E3. In some embodiments, amino acid residues are optionally protected for peptide synthesis (e.g., peptide synthesis using Fmoc-protected amino acids wherein certain side chains may be protected). In some embodiments, one or more stapling are achieved through olefin metathesis. In some embodiments, two or more stapling are formed through one olefin metathesis process. In some embodiments, the present disclosure provides a stapled peptide agent described in Table E2 or Table E3 or a salt thereof (e.g., a pharmaceutically acceptable salt thereof). In some embodiments, the present disclosure provides a stereoisomer of a stapled peptide agent described in Table E2 or Table E3 or a salt thereof (e.g., a pharmaceutically acceptable salt thereof). In some embodiments, the present disclosure provides a E/Z stereoisomer of a stapled peptide agent described in Table E2 or Table E3 or a salt thereof (e.g., a pharmaceutically acceptable salt thereof). In some embodiments, from the N to C direction, an olefin double bond in the first staple that comprising such a bond is Z, and an olefin double in the second staple that comprising such a bond is E (Z-E); in some embodiments, it is (Z-Z); in some embodiments, it is (E-Z); in some embodiments, it is (E-E). In some embodiments, from the N to C direction, an olefin double bond in the first (i, i+2), (i, i+3) or (i, i+4) staple that comprising such a bond is Z, and an olefin double in the first (i, i+7) staple that comprising such a bond is E (Z-E); in some embodiments, it is (Z-Z); in some embodiments, it is (E-Z); in some embodiments, it is (E-E). In some embodiments, an agent comprises an olefin double bond in a third staple, and it is E; in some embodiments, it is Z. In some embodiments, an agent comprises an olefin double bond in a fourth staple, and it is E; in some embodiments, it is Z.

In some embodiments, one or more or all staples are formed after chain extension. In some embodiments, one or more or all staples are formed during chain extension. In some embodiments, one or more or all staples by metathesis are formed after chain extension. In some embodiments, one or more or all staples by metathesis are formed during chain extension.

In some embodiments, the present disclosure provides a method, comprising
a) preparing a first compound comprising two moieties each of which independently comprises an olefin double bond;
b) providing a second compound by stapling the two moieties by olefin metathesis of an olefin double bond of one moiety with an olefin double bond of the other to form a first-formed staple;
c) add one or more additional moieties to the second compound to provide a third compound which comprising two moieties each of which independently comprises an olefin double bond; and
d) providing a fourth compound by stapling the two moieties in the third compound by olefin metathesis of an olefin double bond of one moiety with an olefin double bond of the other to form a second-formed staple.

In some embodiments, a moiety is an amino acid residue. In some embodiments, each moiety is independently an amino acid residue. In some embodiments, each moiety is independently an amino acid residue comprising a terminal olefin as described herein. In some embodiments, there are two olefin double bonds in one moiety, e.g., of the first compound. For example, in some embodiments, such a moiety is B5. In some embodiments, two moieties of a first compound is independently X⁴ and X¹¹. In some embodiments, a first-formed staple is a (i, i+7) staple. In some embodiments, a first compound comprises —X⁴X⁵X⁶X⁷X⁸X⁹X¹⁰X¹¹—. In some embodiments, a first compound comprises —X⁴X⁵X⁶X⁷X⁸X⁹X¹⁰X¹¹X¹²X¹³X¹⁴—. In some embodiments, a first compound comprises a staple. In some embodiments, a staple is a (i, i+4) staple. In some embodiments, a staple is between X¹⁰ and X¹⁴. In some embodiments, an olefin double bond in a third compound is present in the first compound (e.g., an unstapled olefin double bond of B5). In some embodiments, one and only one amino acid residue comprises an olefin double bond is added to the second compound. In some embodiments, ac third compound is or comprises —X¹X²X³X⁴X⁵X⁶X⁷X⁸X⁹X¹⁰X¹¹—. In some embodiments, a third compound is or comprises —X¹X²X³X⁴X⁵X⁶X⁷X⁸X⁹X¹⁰X¹¹X¹²X¹³X¹⁴—. In some embodiments, a first- and second-formed staples are bonded to the same amino acid residue. In some embodiments, a first- and second-formed staples are bonded to the same atom. In some embodiments, a second-formed staple is a (i, i+2), (i, i+3) or (i, i+4) staple. In some embodiments, two moieties in the third compound is independently X¹ and X⁴. In some embodiments, a first-formed staple is formed with E selectivity as described herein (e.g. about 1.1:1, 1.2:1, 1.3:1, 1.4:1, 1.5:1, 2:1, 3:1, 4:1, 5:1, 10:1, 20:1, 30:1, 40:1, 50:1, or more). In some embodiments, a second-formed staple is formed with Z selectivity as described herein (e.g., about 1.1:1, 1.2:1, 1.3:1, 1.4:1, 1.5:1, 2:1, 3:1, 4:1, 5:1, 10:1, 20:1, 30:1, 40:1, 50:1, or more). In some embodiments, synthesis may be performed on a solid support (e.g., solid phase peptide synthesis), and a compound or an agent may be on a solid support. In some embodiments, stapling during chain extension, or individually performed stapling for one or more staples, can provide advantages, e.g., increased selectivity, yield, purity, etc.

In some embodiments, two or more staples are formed in a metathesis reaction. In some embodiments, all staples formed by metathesis are formed in a metathesis reaction. In some embodiments, each of such staples are formed through olefin metathesis of terminal olefins. In some embodiments, multiple staples are formed after full lengths of peptides have been achieved. In some embodiments, one or more staples comprising double bonds are formed after full lengths of peptides have been achieved. In some embodiments, all staples comprising double bonds are formed after full lengths of peptides have been achieved. In some embodiments, one or more staples formed through metathesis are formed after full lengths of peptides have been achieved. In some embodiments, all staples formed through metathesis are formed after full lengths of peptides have been achieved.

In some embodiments, stepwise stapling, in which two or more staples are formed in two or more steps, were performed. In some embodiments, stepwise stapling provides improved levels of selectivity to form a desired product (e.g., I-66) over other compounds, e.g., stereoisomers (e.g., for I-66, I-67). In some embodiments, an improvement is about or at least about 1.5, 2, 2.5, 3, 4, 5, 6, 7, 8, 9, or 10 fold. In some embodiments, an improvement is assessed by comparing percentage of a desired product among all related stereoisomers. In some embodiments, an improvement is assessed by ratios of a desired product versus a stereoisomer (e.g., I-66 versus I-67). In some embodiments, two staples comprising olefin double bonds are formed in two separate steps. In some embodiments, two staples formed by metathesis are formed in two separate steps. In some embodiments, two staples bonded to the same amino acid residue are formed in two separate steps. In some embodiments, two staples bonded to the same atom are formed in two separate steps. In some embodiments, two staples bonded to the same carbon atom are formed in two separate steps. In some embodiments, two staples formed from B5 are formed in two separate steps. In some embodiments, a provided technologies comprise a third step forming a third staple. In some embodiments, each staple is formed in a separate step. In some embodiments, the present disclosure provides a method for preparing a stapled peptide, comprising:
1) reacting a first reactive group with a second reactive group to form a first staple, wherein the first and second reactive groups are in two different amino acid residues; and
2) reacting a third reactive group with a fourth reactive group to form a second staple, wherein the third and fourth reactive groups are in two different amino acid residues.

Alternatively or additionally, in some embodiments, a method comprises reacting a fifth reactive group with a sixth reactive group to form a third staple, wherein the fifth and sixth reactive groups are in two different amino acid residues. In some embodiments, a third staple is formed before a first and second staples.

In some embodiments, a first staple is formed through a metathesis reaction. In some embodiments, each of the first and second reactive groups independently is or comprises a double bond. In some embodiments, each of the first and second reactive groups is independently a terminal olefin. In some embodiments, a first staple is formed through olefin metathesis. In some embodiments, a first staple is an (i, i+7) staple. Various metathesis technologies may be utilized in accordance with the present disclosure to form a first staple. In some embodiments, a metathesis reaction is performed in the presence of a catalyst. In some embodiments, a catalyst is Hoveyda-Grubbs M720 catalyst (CAS 301224-40-8). In some embodiments, a first staple is between X⁴ and X¹¹.

In some embodiments, a second staple is formed through a metathesis reaction. In some embodiments, each of the third and fourth reactive groups independently is or comprises a double bond. In some embodiments, each of the third and fourth reactive groups is independently a terminal olefin. In some embodiments, a second staple is formed through olefin metathesis. In some embodiments, a second staple is an (i, i+3) staple. Various metathesis technologies may be utilized in accordance with the present disclosure to form a second staple. In some embodiments, a metathesis reaction is performed in the presence of a catalyst. In some embodiments, a catalyst is Grubbs M102 catalyst (CAS 172222-30-9). In some embodiments, a second staple is between X¹ and X⁴.

In some embodiments, one of the first and second reactive groups, and one of the third and fourth reactive groups, are in the same amino acid residues. In some embodiments, they are independently in a side chain and the two side chains are bonded to the same atom. In some embodiments, the two side chains are bonded to the same carbon atom, e.g., as in B5. In some embodiments, the first and second staples are bonded to the same amino acid residue. In some embodiments, they are bonded to same atom. In some embodiments, they are bonded to the same carbon, e.g., in B5.

In some embodiments, a third staple comprises an amide group, e.g., —C(O)N(R′)— wherein R′ is as described herein. In some embodiments, a third staple comprises —C(O)NH—. In some embodiments, a third staple is a (i, i+4) staple. In some embodiments, one of the fifth and the sixth reactive groups is or comprises an amino group or an activated form thereof, and the other is or comprises an acid group, e.g., a carboxyl group, or an activated form thereof. In some embodiments, a third staple is formed through an amidation reaction. In some embodiments, a third staple is not formed by a metathesis reaction. In some embodiments, a third staple does not comprise an olefin double bond. Various amidation technologies are available and may be utilized herein. As described herein, other types of staples may be utilized and constructed as well. See, for example, preparation of I-66, 1-335, etc. in the Examples. In some embodiments, a third staple is between X¹⁰ and X¹⁴

In some embodiments, as described herein, one or more stapling steps are independently performed before full lengths are achieved. For example, in some embodiments, a third staple is formed before the two amino acid residues comprising the first and second reactive groups are both installed. Alternatively or additionally, in some embodiments, a first staple is formed before the two amino acid residues comprising the third and fourth reactive groups are both installed. In some embodiments, a third staple is formed after an amino acid residue comprising one of the first and second reactive group is installed but before an amino acid residue comprising the other of the first and second reactive group is installed. In some embodiments, a first staple is formed after an amino acid residue comprising one of the third and fourth reactive group is installed but before an amino acid residue comprising the other of the third and fourth reactive group is installed. In some embodiments, two or more stapling steps are performed based on the positions of the related staples and the directions of peptide synthesis, and one or more staples closer to the starting termini are formed before one or more staples further away from the starting termini. In some embodiments, peptide synthesis is performed from C-terminus to N-terminus. In some embodiments, for a first staple and a second staple, the one that first has both related residues installed is formed first. For example, in a C-terminal to N-terminal peptide synthesis, a staple between X⁴ and X¹¹ is formed before a staple between X¹ and X⁴.

Various metal complexes or catalysts are useful for metathesis. For example, in some embodiments, a metal complex is a Grubbs catalyst. In some embodiments, it is In some embodiments, a metal complex is a Hoveyda-Grubbs catalyst. In some embodiments, it is Grubbs I M102. Hoveyda-Grubbs M720 catalyst. In some embodiments, a catalyst provides product E/Z selectivity. As appreciated by those skilled in the art, catalysts can be utilized at various suitable levels, e.g., about 1%, 2%, 3%, 4%, 5%, 10%, 20%, 25%, 30%, 40%, 50% mol or more.

In some embodiments, the present disclosure provides technologies for controlling ratio of E/Z isomers of one or more or each olefin double bond formed during olefin metathesis. In some embodiments, one or more or each olefin double bond is formed with a isomer ratio of about 1.1:1, 1.2:1, 1.3:1, 1.4:1, 1.5:1, 2:1, 3:1, 4:1, 5:1, 10:1, 20:1, 30:1, 40:1, 50:1, or more. In some embodiments, in a product composition one or more or each olefin double bond has an isomer ratio of about 1.1:1, 1.2:1, 1.3:1, 1.4:1, 1.5:1, 2:1, 3:1, 4:1, 5:1, 10:1, 20:1, 30:1, 40:1, 50:1, or more. In some embodiments, it is independently about 1.5:1 or more. In some embodiments, it is independently about 2:1 or more. In some embodiments, it is independently about 3:1 or more. In some embodiments, it is independently about 4:1 or more. In some embodiments, it is independently about 5:1 or more. In some embodiments, it is independently about 6:1 or more. In some embodiments, it is independently about 7:1 or more. In some embodiments, it is independently about 8:1 or more. In some embodiments, it is independently about 9:1 or more. In some embodiments, it is independently about 10:1 or more. In some embodiments, it is independently about 20:1 or more. In some embodiments, it is independently about 30:1 or more. In some embodiments, it is independently about 40:1 or more. In some embodiments, it is independently about 50:1 or more. In some embodiments, a ratio is E: Z. In some embodiments, a ratio is Z: E.

In some embodiments, stapling creates one or more chiral centers. For example, in some embodiments, when B5 forms two staples with two other amino acid residues, a chiral center may form. In some embodiments, a formed chiral center is R in an agent. In some embodiments, a formed chiral center is S in an agent. In some embodiments, a composition comprises both agents being R and S at a chiral center. In some embodiments, a chiral center is formed with stereoselectivity (e.g., in some embodiments, diastereoselectivity when other chiral elements are present in the same molecule). In some embodiments, the selectivity is about or at least about 60%, 65%, 70%, 75%, 80%, 85%, 90%, 95%, 98% or 99% (when selectivity is 98%, 98% of all product molecules share the same stereochemistry at the chiral center.). In some embodiments, in a composition described herein, e.g., a pharmaceutical composition, about or at least about 60%, 65%, 70%, 75%, 80%, 85%, 90%, 95%, 98% or 99% of all molecules having the same constitution and salts thereof share the same stereochemistry at a chiral center, e.g., a chiral center bonded to two staples (e.g., in B5). In some embodiments, it is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about or at least about 98%. In some embodiments, it is about or at least about 99%.

In some embodiments, an olefin double bond in a staple may be further modified. In some embodiments, an olefin double bond in a staple is hydrogenated thus converting it into a single bond. In some embodiments, a modification is epoxidation. In some embodiments, a modification is halogenation. Those skilled in the art appreciate that various other modifications are suitable for olefin double and can be utilized in accordance with the present disclosure.

In some embodiments, crude product compositions are purified, e.g., through chromatography technologies such as liquid chromatography. In some embodiments, one or more product compositions are collected based on separated portions, e.g., HPLC peaks, with the correct observed mass. In some embodiments, each product composition independently corresponds to a different peak (e.g., in some embodiments, by UV detection at a suitable wavelength, e.g., 220 nm) with the correct observed mass. In some embodiments, a peak area of one or more or each product composition is independently about 5%, 10%, 20%, 30%, 40%, 50%, 60%, 70%, 80%, 90% or more of the total peak area of all peak(s) with the correct mass. In some embodiments, it is about 5% or more. In some embodiments, it is about 10% or more. In some embodiments, it is about 20% or more. In some embodiments, it is about 25% or more. In some embodiments, it is about 30% or more. In some embodiments, it is about 40% or more. In some embodiments, it is about 50% or more. In some embodiments, a product composition comprises one isomer. In some embodiments, a product composition comprises two or more isomers (e.g., those that cannot be sufficiently separated). In some embodiments, each product composition independently has a purity and/or stereopurity as described herein, e.g., in some embodiments, for one or more (e.g., 1, 2, 3, 4, 5 or more) or each olefin double bond in a staple, the ratio of the two stereoisomers is independently about 1.1:1, 1.2:1, 1.3:1, 1.4:1, 1.5:1, 2:1, 3:1, 4:1, 5:1, 10:1, 20:1, 30:1, 40:1, 50:1, 60:1, 70:1, 80:1, 90:1, 100:1 or more. In some embodiments, ratios may be assessed by NMR, HPLC, etc.

In some embodiments, as described herein, certain stapled peptides, and in particular cysteine stapled peptides, may be provided in and/or produced by a biological system and reacting with a provided reagent, e.g., one having the structure of R^{x}-L^{s2}-R^{x}, or a salt thereof, wherein R^{x} can react with —SH groups under suitable conditions. In some embodiments, each R^{x} is a suitable leaving group. In some embodiments, each R^{x} is independently —Br.

In some embodiments, peptides are prepared on solid phase on a synthesizer using, typically, Fmoc chemistry. In some embodiments, the present disclosure provides protected and/or activated amino acids for synthesis.

In some embodiments, staples are formed by olefin metathesis. In some embodiments, a product double bond of metathesis is reduced/hydrogenated. In some embodiments, CO₂ are extruded from a carbamate moiety of a staple. In some embodiments, provided stapled peptides are further modified, and/or conjugated to other entities. Conditions and/or reagents of these reactions are widely known in the art and can be performed in accordance with the present disclosure to provide stapled peptides.

Properties and/or activities of provided stapled peptides can be readily assessed in accordance with the present disclosure, for example, through use of one or more methods described in the examples.

In some embodiments, technologies for preparing and/or assessing stapled peptides include those described in U.S. Pat. No. 9,617,309, US 2015-0225471, US 2016-0024153, US 2016-0215036, US2016-0244494, WO 2017/062518, etc. In some embodiments, technologies for preparing and/or assessing stapled peptides are described in WO 2022/261257 and are incorporated herein by reference.

In some embodiments, the present disclosure provides products manufactured and/or characterized by processes and/or technologies described herein.

Provided compounds can be provided in high purity. In some embodiments, a provided compound is at least about 85%, 90%, 91%, 92%, 93%, 94%, 95%, 96%, 97%, 98%, or 99% pure. In some embodiments, provided compounds, e.g., amino acids optionally protected/activated, are essentially free of impurities, including stereoisomers.

In some embodiments, an agent may have one or more stereoisomers which may independently co-exist in a composition or preparation. In some embodiments, a provided agent has a stereopurity of about or at least about 80%, 85%, 90%, 91%, 92%, 93%, 94%, 95%, 96%, 97%, 98%, or 99%. In some embodiments, it is about or at least about 80%. In some embodiments, it is about or at least about 85%. In some embodiments, it is about or at least about 90%. In some embodiments, it is about or at least about 95%. In some embodiments, it is about or at least about 96%. In some embodiments, it is about or at least about 97%. In some embodiments, it is about or at least about 98%. In some embodiments, it is about or at least about 99%. In some embodiments, stereoisomers are essentially free from a preparation or composition (e.g., cannot be reliably observed in NMR or HPLC). In some embodiments, an agent comprises one or more staples independently comprising one or more olefin double bond. In some embodiments, stereopurity is with respect to E/Z stereoisomers. In some embodiments, for one or more (e.g., 1, 2, 3, 4, 5 or more) or each olefin double bond in a staple, the ratio of the two stereoisomers is independently about 1.1:1, 1.2:1, 1.3:1, 1.4:1, 1.5:1, 2:1, 3:1, 4:1, 5:1, 10:1, 20:1, 30:1, 40:1, 50:1, 60:1, 70:1, 80:1, 90:1, 100:1 or more. In some embodiments, it is independently about 1.5:1 or more. In some embodiments, it is independently about 2:1 or more. In some embodiments, it is independently about 3:1 or more. In some embodiments, it is independently about 4:1 or more. In some embodiments, it is independently about 5:1 or more. In some embodiments, it is independently about 6:1 or more. In some embodiments, it is independently about 7:1 or more. In some embodiments, it is independently about 8:1 or more. In some embodiments, it is independently about 9:1 or more. In some embodiments, it is independently about 10:1 or more. In some embodiments, it is independently about 20:1 or more. In some embodiments, it is independently about 30:1 or more. In some embodiments, it is independently about 40:1 or more. In some embodiments, it is independently about 50:1 or more. In some embodiments, it is independently about 60:1 or more. In some embodiments, it is independently about 70:1 or more. In some embodiments, it is independently about 80:1 or more. In some embodiments, it is independently about 90:1 or more. In some embodiments, it is independently about 100:1 or more. In some embodiments, a ratio is E: Z. In some embodiments, a ratio is Z: E. Those skilled in the art appreciate that E and Z isomers may be selectively enriched through modulating manufacturing processes, purification, staple positioning and/or lengths, etc.

In some embodiments, the present disclosure provides compounds, agents, preparations, compositions, etc., that have low metal levels. In some embodiments, the present disclosure provides compounds, agents, preparations, compositions, etc., that have low ruthenium levels. In some embodiments, the present disclosure provides compounds, agents, preparations, compositions, etc., that have low palladium levels. In some embodiments, a level is no more than about 1000, 500, 200, 100, 90, 80, 70, 60, 50, 40, 30, 20, 10, 5, 4, 3, 2, or 1 ppm (weight/weight). In some embodiments, a level is no more than about 1000 ppm. In some embodiments, a level is no more than about 500 ppm. In some embodiments, a level is no more than about 100 ppm. In some embodiments, a level is no more than about 50 ppm. In some embodiments, a level is no more than about 20 ppm. In some embodiments, a level is no more than about 10 ppm. As appreciated by those skilled in the art, various technologies can be utilized to assess metal levels. In some embodiments, a metal level, e.g., a ruthenium level, a palladium level, etc., is assessed according to USP <233>. In some embodiments, ICP-MS is utilized. In some embodiments, ICP-OES is utilized.

### Compositions

Among other things, the present disclosure provides compositions that comprise or otherwise relate to provided agents, e.g., small molecule agents, peptide agents (e.g., stapled peptides), as described herein.

In some embodiments, provided compositions are or comprise an assay system for characterizing (and optionally including) a stapled peptide as described herein.

In some embodiments, provided compositions are pharmaceutical compositions e.g., that comprise or deliver one or more provided agents. In some embodiments, an agent is in a pharmaceutically acceptable salt form.

In some embodiments, an agent is a peptide. In some embodiments, an agent is a stapled peptide. In some embodiments, an agent comprises a detectable moiety, e.g., fluorescent moiety, radioactive moiety, biotin, etc. In some embodiments, a detectable moiety is directly detectable. In some embodiments, a detectable antibody is detected indirectly, e.g., utilizing an antibody, an agent that can reacting with a detectable moiety to form a detectable product, etc.

In some embodiments, a pharmaceutical composition comprises a provided agent and a pharmaceutically acceptable excipient (e.g., carrier).

In some embodiments, a peptide composition may include or deliver a particular form (e.g., a particular optical isomer, diastereomer, salt form, covalent conjugate form [e.g., covalently attached to a carrier moiety], etc., or combination thereof) of an agent as described herein). In some embodiments, an agent included or delivered by a pharmaceutical composition is described herein is not covalently linked to a carrier moiety.

In some embodiments, multiple stereoisomers exist for an agent that contains chiral centers and/or double bonds. In some embodiments, level of a particular agent in a composition is enriched relative to one or more or all of its stereoisomers. For example, in some embodiments, a particularly configuration of a double bond (E/Z) is enrich. In some embodiments, for each double bond a configuration is independently enriched. In some embodiments, for a chiral element, e.g., a chiral center, one configuration is enriched. In some embodiments, for a chiral center bonded to two staples, one configuration is enriched. In some embodiments, for each chiral element a configuration is independently enriched. In some embodiments, for one or more or all stereochemical element (e.g., double bonds, chiral element, etc.), one configuration is independently enriched. In some embodiments, for each double bond in each staple, one configuration is independently enriched. In some embodiments, for each double bond in each staple, one configuration is independently enriched, and for a chiral center bonded to two staples, one configuration is enriched. In some embodiments, enrichment for each double bond is independently E or Z. In some embodiments, enrichment for each chiral element is independently R or S. In some embodiments, enrichment for each stereochemical element, e.g., double bond, chiral center, etc., is about or at least about a certain level, e.g., 60%, 65%, 70%, 75%, 80%, 85%, 90%, 91%, 92%, 93%, 94%, 95%, 96%, 97%, 98%, or 99% (percentage of an agent). In some embodiments, about or at least about a certain level, e.g., 60%, 65%, 70%, 75%, 80%, 85%, 90%, 91%, 92%, 93%, 94%, 95%, 96%, 97%, 98%, or 99% of all molecules in a composition that share the constitution of an agent or a salt thereof are the agent or a salt thereof. In some embodiments, a level is about or at least about 60%. In some embodiments, it is about or at least about 65%. In some embodiments, it is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about or at least about 96%. In some embodiments, it is about or at least about 97%. In some embodiments, it is about or at least about 98%. In some embodiments, it is about or at least about 99%.

In some embodiments, a provided therapeutic composition may comprise one or more additional therapeutic agents and/or one or more stabilizing agents and/or one or more agents that alters (e.g., extends or limits to a particular tissue, location or site) rate or extent of delivery over time.

In some embodiments, a composition is a pharmaceutical composition which comprises or delivers a provided agent (e.g., a stapled peptide) or a pharmaceutically acceptable salt thereof and a pharmaceutically acceptable excipient. In some embodiments, a composition comprises one and only stereoisomer of an agent (e.g., a stapled peptide) and/or one or more salts thereof. In some embodiments, a composition comprises two or more stereoisomers of an agent (e.g., a stapled peptide) and/or one or more salts thereof. In some embodiments, the two or more stereoisomers of an agent (e.g., a stapled peptide) or salts thereof elute as a single peak (e.g., UV and/or MS detection) in a chromatography, e.g., HPLC.

### Uses and Applications

Provided agents and compositions can be utilized for various purposes. For example, certain compounds may be utilized as amino acids, either directly or for preparation of other compounds such as peptides. Certain agents, e.g., peptides, may be utilized to prepare stapled peptides. Certain agents that are or comprise peptides, particularly stapled peptides, and compositions thereof, are biologically active and can be utilized for various purposes, e.g., as therapeutics toward various conditions, disorders or diseases, as tools for modulating biological functions, etc.

In some embodiments, the present disclosure provides agents and compositions thereof for modulating beta-catenin functions. In some instances, beta-catenin is reported to have multiple cellular functions including regulation and coordination of cell-cell adhesion and gene transcription. In some embodiments, agents described herein may inhibit beta-catenin activity and/or level and may, for example, inhibit neoplastic growth. In some embodiments, agents described herein may activate and/or increase level of beta-catenin and may, for example, be used to treat male pattern baldness or alopecia.

It is reported that beta-catenin can interact with members of the TCF/LEF family at a TCF site on beta-catenin. In some embodiments, provided technologies can decrease, suppress or block one or more of such interactions. In some embodiments, the present disclosure provides methods for modulating an interaction between beta-catenin and its binding partner (e.g., a TCF/LEF family member) comprising contacting beta-catenin with a provided agent.

In some embodiments, binding of provided agents to beta-catenin competes or inhibits binding of another agent. In some embodiments, binding of provided agents to beta-catenin competes or inhibits binding of another agent. In some embodiments, binding of provided agents to beta-catenin competes or inhibits binding of TCF or a fragment thereof.

In some embodiments, provided agents compete with TCF7, LEF1, TCF7L1, TCF7L2, Axin1, Axin2, APC, CDH1, or CDH2, or a fragment thereof, for beta-catenin binding.

In some embodiments, provided agents interfere with interactions of TCF7, LEF1, TCF7L1, TCF7L2, Axin1, Axin2, APC, CDH1, or CDH2, or a fragment thereof, with beta-catenin.

In some embodiments, provided technologies can reduce or block beta-catenin's interactions with all TCF family members, E-cadherin and APC, but did not significantly affect its interactions with ICAT, AXIN and BCL9. In some embodiments, provided technologies can interrupt beta-catenin/TCF interaction at both physical interaction level (e.g., as confirmed by NanoBRET, co-IP, etc.) and transcriptional level (e.g., as confirmed by reporter cell line, endogenous gene expression, etc.). In some embodiments, provided technologies show no effect on beta-catenin stability.

In some embodiments, the present disclosure provides methods for modulating interactions of beta-catenin with a partner, e.g., TCF7, LEF1, TCF7L1, TCF7L2, Axin1, Axin2, APC, CDH1, or CDH2, or a fragment thereof, comprising contacting beta-catenin with a provided agent or a composition that comprises or delivers a provided agent. In some embodiments, the present disclosure provides methods for modulating interactions of beta-catenin with a partner, e.g., TCF7, LEF1, TCF7L1, TCF7L2, Axin1, Axin2, APC, CDH1, or CDH2, or a fragment thereof, comprising administering or delivering to a system comprising beta-catenin and the partner a provided agent or a composition that comprises or delivers a provided agent. In some embodiments, a system is an in vitro system. In some embodiments, a system is an in vivo system. In some embodiments, a system is or comprises a cell, tissue or organ. In some embodiments, a system is a subject. In some embodiments, the present disclosure provides method for inhibiting cell growth, comprising administering or delivering to a population of cells an effective amount of a provided agent or a pharmaceutically acceptable salt thereof. In some embodiments, the present disclosure provides method for killing cells associated with a condition, disorder or disease (e.g., cancer), comprising administering or delivering to a population of such cells an effective amount of a provided agent or a pharmaceutically acceptable salt thereof.

In some embodiments, a system, cell, tissue, organ or subject comprises or expresses a mutant beta-catenin. In some embodiments, a cell is a hepatocellular cell that comprises or expresses a mutant beta-catenin. In some embodiments, a cell is a hepatocellular cancer cell that comprises or expresses a mutant beta-catenin. In some embodiments, a system, tissue, organ or subject comprises a hepatocellular cell that comprises or expresses a mutant beta-catenin. In some embodiments, a system, cell, tissue, organ or subject comprises a hepatocellular cancer cell that comprises or expresses a mutant beta-catenin. For example, in some embodiments, a mutation is D32V. In some embodiments, a mutation is D32H. In some embodiments, a mutation is S45F. In some embodiments, a mutation is S37C. In some embodiments, a mutant beta-catenin is D32V beta-catenin. In some embodiments, a mutant beta-catenin is D32H beta-catenin. In some embodiments, a mutant beta-catenin is S45F beta-catenin. In some embodiments, a mutant beta-catenin is S37C beta-catenin. In some embodiments, a mutation is associated with a condition, disorder or disease, e.g., cancer (e.g., hepatocellular cancer).

In some embodiments, the present disclosure provides methods for preventing a condition, disorder or disease associated with beta-catenin (e.g., a cancer, a neurodegenerative disease, etc.), comprising administering or delivering to a subject susceptible thereto an effective amount of a provided agent or a pharmaceutically acceptable salt thereof. In some embodiments, the present disclosure provides methods for treating a condition, disorder or disease associated with beta-catenin (e.g., aberrant beta-catenin activity and/or expression level), comprising administering or delivering to a subject suffering therefrom an effective amount of a provided agent or a pharmaceutically acceptable salt thereof. In some embodiments, a provided agent is administered as a pharmaceutical composition that comprises or delivers an effective amount of a provided agent or a pharmaceutically acceptable salt thereof. In some embodiments, a condition, disorder or disease is associated with beta-catenin interaction with a partner, e.g., TCF7, LEF1, TCF7L1, TCF7L2, Axin1, Axin2, APC, CDH1, and/or CDH2. In some embodiments, a condition, disorder or disease is associated with beta-catenin with TCF. In some embodiments, a condition, disorder or disease is cancer. In some embodiments, provided agents may be administered in combination with another therapy, e.g., immunotherapy. In some embodiments, a condition, disorder, or disease is selected from cancer, cardiac disease, dilated cardiomyopathy, fetal alcohol syndrome, depression, and diabetes. In some embodiments, a condition, disorder, or disease is a heart condition, disorder, or disease. In some embodiments, a condition, disorder, or disease is cancer. In some embodiments a cancer is selected from: colon cancer, colorectal cancer, rectal cancer, prostate cancer familial adenomatous polyposis (FAP), Wilms Tumor, melanoma, hepatocellular carcinoma, ovarian cancer, endometrial cancer, medulloblastoma pilomatricomas, primary hetpatocellular carcinoma, ovarial carcinoma, breast cancer, lung cancer, glioblastoma, pliomatrixoma, medulloblastoma, thyroid tumors, and ovarian neoplasms. In some embodiments, a condition, disorder or disease is a cancer, e.g., colorectal cancer, hepatocellular cancer, melanoma, gastric cancer, bladder cancer, and endometrial cancer. In some embodiments, a cancer is colorectal cancer. In some embodiments, a cancer is hepatocellular cancer. In some embodiments, a cancer is prostate cancer. In some embodiments, a cancer is melanoma. In some embodiments, a subject comprises a mutation in beta-catenin, e.g., D32V, D32H, etc. In some embodiments, a subject comprises a mutation in beta-catenin, e.g., S45F, S37C, etc. In some embodiments, a subject comprises or expresses beta-catenin D32V. In some embodiments, a subject comprises or expresses beta-catenin D32H. In some embodiments, a subject comprises or expresses beta-catenin S45F. In some embodiments, a subject comprises or expresses beta-catenin S37C. In some embodiments, a mutant beta-catenin is or is expressed in a hepatocellular cell. In some embodiments, a mutant beta-catenin is or is expressed in a cancer cell. In some embodiments, a mutant beta-catenin is or is expressed in a hepatocellular cancer cell. In some embodiments, mutations in beta-catenin are present in cells, models, tissues, organs, systems, subjects, etc., with other mutations, e.g., those associated with various conditions, disorders or diseases such as cancer. For example, in some embodiments, a beta-catenin mutation is present together with a MSH6 mutation. In some embodiments, a cancer is MSI-H. In some embodiments, a cancer is MSI-H and comprises a beta-catenin mutation optionally with another mutation, e.g., a MSH6 mutation.

Among other things, provided technologies can provide effective tumor size reduction across a period of time, e.g., in some embodiments, about or at least about 10, 20, 30, 40, 50, 60, 70 days, or about or at least about 1, 2, 3, 4, 5, 6, 7, 8, 9, 10, 11 or 12 months. In some embodiments, provided technologies can reduce tumor growth across a period of time, e.g., in some embodiments, about or at least about 10, 20, 30, 40, 50, 60, 70 days, or about or at least about 1, 2, 3, 4, 5, 6, 7, 8, 9, 10, 11 or 12 months. In some embodiments, provided technologies substantially prevent tumor growth across a period of time, e.g., in some embodiments, about or at least about 10, 20, 30, 40, 50, 60, 70 days, or about or at least about 1, 2, 3, 4, 5, 6, 7, 8, 9, 10, 11 or 12 months. In some embodiments, reduction of tumor size, and/or reduction or prevention of tumor growth, is observed in an animal model, e.g., those described herein. In some embodiments, reduction of tumor size, and/or reduction or prevention of tumor growth, is observed in a subject, e.g., a cancer patient. In some embodiments, reduction of tumor size, and/or reduction or prevention of tumor growth, is observed when one or more doses are administered. In some embodiments, reduction of tumor size, and/or reduction or prevention of tumor growth, is observed and/or maintained after a dose. In some embodiments, reduction of tumor size, and/or reduction or prevention of tumor growth, is observed and/or maintained after a last dose, e.g., about or at least about 10, 20, 30, 40, 50, or 60 days, or about or at least about 1, 2, 3, 4, 5, 6, 7, 8, 9, 10, 11 or 12 months, after a last dose.

In some embodiments, the present disclosure provides technologies for modulating level of expression and/or activity of a nucleic acid, e.g., a gene, a transcript, a polypeptide, and/or a product thereof in a system, comprising administering or delivering to the system a provided agent or a composition that comprises or delivers a provided agent. In some embodiments, level of expression of a nucleic acid, e.g., a gene, or a product thereof (e.g., a transcript, a polypeptide, etc.) is modulated. In some embodiments, level of activity of a nucleic acid, e.g., a gene, or a product thereof (e.g., a transcript, a polypeptide, etc.) is modulated. In some embodiments, level of a transcript and/or a product thereof (e.g., a polypeptide) is modulated. In some embodiments, level of activity of a transcript and/or a product thereof (e.g., a polypeptide) is modulated. In some embodiments, a transcript is a transcript of a nucleic acid, e.g., gene, described herein. In some embodiments, level of a polypeptide is modulated. In some embodiments, level of activity of a polypeptide is modulated. In some embodiments, a polypeptide is a encoded by a nucleic acid or a transcript described herein. In some embodiments, a level is increased. In some embodiments, a level is decreased. As described herein, in some embodiments, a system is an in vitro system. In some embodiments, a system is an in vivo system. In some embodiments, a system is or comprises a cell, tissue or organ. In some embodiments, a system is or comprises one or more cancer cells. In some embodiments, a system is or comprises tumor. In some embodiments, a system is or comprises an organism. In some embodiments, a system is a subject. In some embodiments, a system is a human. In some embodiments, a system comprises beta-catenin. In some embodiments, a system expresses beta-catenin. In some embodiments, a system comprises beta-catenin and a partner. In some embodiments, a system expresses beta-catenin and a partner. In some embodiments, a level is regulated by beta-catenin. In some embodiments, a level is regulated by WNT activation. In some embodiments, a level is regulated by beta-catenin/WNT signaling. In some embodiments, a level is regulated by interaction of beta-catenin and a partner. In some embodiments, interaction of beta-catenin and a partner is modulated, e.g., reduced, prevented, etc., by an agent, e.g., a stapled peptide, as described herein. For example, in some embodiments, a partner is TCF. In some embodiments, level of expression and/or activity of a nucleic acid and/or a product thereof is modulated. In some embodiments, a nucleic acid is AXIN2. In some embodiments, level of an AXIN2 transcript, e.g., mRNA, is reduced. In some embodiments, level of an AXIN2 polypeptide is reduced. In some embodiments, a nucleic acid is SP5. In some embodiments, level of an SP5 transcript, e.g., mRNA, is reduced. In some embodiments, level of an SP5 polypeptide is reduced. In some embodiments, a nucleic acid is CXCL12. In some embodiments, level of a CXCL12 transcript, e.g., mRNA, is increased. In some embodiments, level of a CXCL12 polypeptide is increased. In some embodiments, a nucleic acid is a member of a negatively enriched gene set observed in, or can be identified using technologies in, e.g., WO 2022/261257. In some embodiments, a nucleic acid is a member of BCAT_GDS748_UP gene set. In some embodiments, a nucleic acid is a member of BCAT.100_UP.V1_UP gene set. In some embodiments, a nucleic acid is a member of HALLMARK_WNT_BETA-CATENIN_SIGNALING gene set. In some embodiments, a nucleic acid is a member of RASHI-RESPONSE_TO_IONIZING-RADIATION_1 gene set. In some embodiments, a nucleic acid is a member of REACTOME-RRNA_PROCESSING gene set. In some embodiments, a nucleic acid is a member of HALLMARK_MYC_TARGETS_V1 gene set. In some embodiments, a nucleic acid is a member of HALLMARK_MYC_TARGETS_V2 gene set. In some embodiments, a nucleic acid is a member of HALLMARK_OXIDATIVE_PHOSPHORYLATION gene set. In some embodiments, a nucleic acid is a member of HALLMARK_E2F_TARGETS gene set. In some embodiments, a nucleic acid is a member of HALLMARK_TNFA_SIGNALING_VIA_NFKB gene set. In some embodiments, a gene set is described in a Figure or an Example. In some embodiments, a nucleic acid is described in a Figure or an Example, or is a member of a gene set is described in a Figure or an Example. Description of various gene sets can be found publicly, e.g., www.gsea-msigdb.org/gsea/msigdb/. In some embodiments, one or more or some or a majority of but not all nucleic acids or genes in a gene set is impacted in the same way, but overall a gene set can be negatively or positively enriched

In some embodiments, the present disclosure provides technologies for detecting, monitoring and/or confirming efficacy of an agent, e.g., a stapled peptide, or a method, e.g., a method of treating a condition, disorder or disease, a method for modulating level of a transcript and/or a product and/or activity thereof, comprising assessing level of expression and/or activity of a nucleic acid, e.g., a gene, a transcript, a polypeptide, and/or a product thereof. In some embodiments, the present disclosure provides technologies for detecting, monitoring and/or confirming efficacy of an agent, e.g., a stapled peptide, comprising administering the agent to a subject, and assessing level of expression and/or activity of a nucleic acid, e.g., a gene, a transcript, a polypeptide, and/or a product thereof, in the subject. In some embodiments, the present disclosure provides technologies for detecting, monitoring and/or confirming efficacy of a method for treating a condition, disorder or disease in a subject, comprising assessing level of expression and/or activity of a nucleic acid, e.g., a gene, a transcript, a polypeptide, and/or a product thereof, in the subject. In some embodiments, a method is a method for treating a condition, disorder or disease associated with TCF-beta-catenin interaction in a subject. In some embodiments, a condition, disorder or disease is cancer as described herein. In some embodiments, the present disclosure provides technologies for selecting subjects for administration or delivery of an agent, e.g., stapled peptide agents described herein (e.g., for preventing or treating a condition, disorder or disease). In some embodiments, the present disclosure provides technologies for selecting subjects for continued administration or delivery of an agent, e.g., stapled peptide agents described herein (e.g., for preventing or treating a condition, disorder or disease) after one or more administrations or deliveries. In some embodiments, level of a transcript is assessed. In some embodiments, level of a polypeptide is assessed. In some embodiments, assessment is performed utilizing a sample or samples collected from a system or a subject. In some embodiments, a sample is collected during administration or delivery. In some embodiments, a sample is collected after administration or delivery. As described herein, in some embodiments, level of expression and/or activity of a nucleic acid and/or a product thereof is modulated. In some embodiments, a nucleic acid is AXIN2. In some embodiments, level of an AXIN2 transcript, e.g., mRNA, is reduced. In some embodiments, level of an AXIN2 polypeptide is reduced. In some embodiments, a nucleic acid is SP5. In some embodiments, level of an SP5 transcript, e.g., mRNA, is reduced. In some embodiments, level of an SP5 polypeptide is reduced. In some embodiments, a nucleic acid is CXCL12. In some embodiments, level of a CXCL12 transcript, e.g., mRNA, is increased. In some embodiments, level of a CXCL12 polypeptide is increased. In some embodiments, a nucleic acid is a member of a gene set as described herein. In some embodiments, a nucleic acid is a member of BCAT_GDS748_UP gene set. In some embodiments, a nucleic acid is a member of BCAT.100_UP.V1_UP gene set. In some embodiments, a nucleic acid is a member of

HALLMARK_WNT_BETA-CATENIN_SIGNALING gene set. In some embodiments, a nucleic acid is a member of RASHI-RESPONSE_TO_IONIZING-RADIATION_1 gene set. In some embodiments, a nucleic acid is a member of REACTOME-RRNA_PROCESSING gene set. In some embodiments, a nucleic acid is a member of HALLMARK_MYC_TARGETS_V1 gene set. In some embodiments, a nucleic acid is a member of HALLMARK_MYC_TARGETS_V2 gene set. In some embodiments, a nucleic acid is a member of HALLMARK_OXIDATIVE_PHOSPHORYLATION gene set. In some embodiments, a nucleic acid is a member of HALLMARK_E2F_TARGETS gene set. In some embodiments, a nucleic acid is a member of HALLMARK_TNFA_SIGNALING_VIA_NFKB gene set. In some embodiments, a gene is CCND2. In some embodiments, a gene is WNT5B. In some embodiments, a gene is AXIN2. In some embodiments, a gene is NKD1. In some embodiments, a gene is WNT6. In some embodiments, a gene is DKK1. In some embodiments, a gene is DKK4. In some embodiments, expression of such a nucleic acid, e.g., a gene, is reduced. In some embodiments, level of a product of such a nucleic acid, e.g., a transcript (e.g., mRNA), a polypeptide, etc., is reduced. In some embodiments, level of activity of a product of such a nucleic acid, e.g., a transcript (e.g., mRNA), a polypeptide, etc., is reduced. In some embodiments, a nucleic acid is a member of a positively enriched gene set observed in, or can be identified using technologies in, e.g., Example 17. In some embodiments, if one or more desired reductions of expression and/or levels of transcripts and/or products thereof, and/or one or more desired negatively and/or positively enriched gene sets, are observed, administration or delivery continues. In some embodiments, administration or delivery continues as prior one(s). In some embodiments, administration or delivery continue with an adjusted dose level and/or regimen. In some embodiments, if desired reductions of expression and/or levels of transcripts and/or products thereof, and/or one or more desired negatively and/or positively enriched gene sets, are not observed, administration or delivery may be adjusted, and in some embodiments, discontinued. In some embodiments, as described herein, desired reductions of expression and/or levels of transcripts and/or products thereof comprise reductions of expression and/or levels of transcripts and/or products thereof of one or more or a majority of or all of SP5, CCND2, WNT5B, AXIN2, NKD1, WNT6, DKK1 and DKK4, nucleic acids of BCAT_GDS748_UP, BCAT.100_UP.VI_UP, HALLMARK_WNT_BETA-CATENIN_SIGNALING, RASHI-RESPONSE_TO_IONIZING-RADIATION_1, REACTOME-RRNA_PROCESSING, HALLMARK_MYC_TARGETS_V1, HALLMARK_MYC_TARGETS_V2, HALLMARK_OXIDATIVE_PHOSPHORYLATION, HALLMARK_E2F_TARGETS, and HALLMARK_TNFA_SIGNALING_VIA_NFKB. In some embodiments, as described herein, desired increase of expression and/or levels of transcripts and/or products thereof comprise increase of expression and/or levels of transcripts and/or products thereof of CXCL12. In some embodiments, desired gene set enrichments comprise negative enrichment of one or more or all of BCAT_GDS748_UP, BCAT.100_UP.V1_UP, HALLMARK_WNT_BETA-CATENIN_SIGNALING, RASHI-RESPONSE_TO_IONIZING-RADIATION_1, REACTOME-RRNA_PROCESSING, HALLMARK_MYC_TARGETS_V1, HALLMARK_MYC_TARGETS_V2, HALLMARK_OXIDATIVE_PHOSPHORYLATION, HALLMARK_E2F_TARGETS, and HALLMARK_TNFA_SIGNALING_VIA_NFKB. Those skilled in the art, e.g., those skilled in relevant clinical fields, reading the present disclosure will appreciate how to make decisions in accordance with the present disclosure.

In some embodiments, comparison is made to a reference. For example, reduction, increase, enrichment (negative or positive), changes, etc., are typically made to a suitable reference. In some embodiments, reduction, increase, enrichment (negative or positive), changes, etc., are to a reference assessment, in some embodiments, of a reference sample. In some embodiments, a reference assessment is or comprises assessment conducted prior to an administration or delivery of an agent. In some embodiments, a reference sample is collected prior to an administration or delivery of an agent. In some embodiments, a reference assessment is or comprises assessment conducted during an administration or delivery of an agent. In some embodiments, a reference sample is collected during an administration or delivery of an agent. In some embodiments, a reference assessment is or comprises assessment conducted after an administration or delivery of an agent. In some embodiments, a reference sample is collected after an administration or delivery of an agent. In some embodiments, a reference assessment is or comprises assessment conducted after an earlier administration or delivery of an agent. In some embodiments, a reference sample is collected after earlier an administration or delivery of an agent.

In some embodiments, a sample is an aliquot of material obtained or derived from a source of interest as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample comprise cancer cells. In some embodiments, a sample is obtained from a tumor. In some embodiments, a sample is obtained from a tumor in a patient.

In some embodiments, levels of two or more transcripts and/or products thereof may be assessed. In some embodiments, assessment is performed after one or more doses of agents, e.g., stapled peptides are administered or delivered to a subject. In some embodiments, if profiles, e.g., reduction, increase, etc., of one or more transcripts and/or products thereof matches those described herein, administration or delivery to a subject may continue. In some embodiments, if profiles, e.g., reduction, increase, etc., of one or more transcripts and/or products thereof matches those described herein, administration or delivery to a subject may be stopped and/or continued according to different dose levels and/or regimens.

Various technologies can be utilized in accordance with the present disclosure to formulate, distribute, administer or deliver provided technologies such as agents, peptides, compounds, compositions, etc. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, provided technologies are administered intravenously.

Among other things, the present disclosure provides various structural moieties including designed amino acid residues that can be utilized to optimize various properties and activities, stability, delivery, pharmacodynamics, pharmacokinetics, etc. to provide various dosage forms, dosage regimen, therapeutic windows, etc. In some embodiments, provided agents and compositions thereof may be utilized with improved dosage regimen and/or unit doses. In some embodiments, administration of provided agents are adjusted based on conditions, disorders or diseases and/or subpopulations. In some embodiments, administration and/or dosage regimen of provided technologies are adjusted according to certain biomarkers and genomic alterations.

Provided agents may deliver biological effects, e.g., therapeutic effects, via various mechanisms. In some embodiments, efficacy may be driven by AUC. In some embodiments, efficacy may be driven by Cmax.

In some embodiments, a provided agent is utilized in combination with another therapy. In some embodiments, a provided agent is utilized in combination with another therapeutic agent. In some embodiments, another therapy or therapeutic agent is administered prior to an administration or delivery of a provided agent. In some embodiments, another therapy or therapeutic agent is administered at about the same time as an administration or delivery of a provided agent. In some embodiments, a provided agent and another agent is in the same pharmaceutical composition. In some embodiments, another therapy or therapeutic agent is administered subsequently to an administration or delivery of a provided agent. In some embodiments, a subject is exposed to both a provided agent and another therapeutic agent. In some embodiments, both a provided agent and another agent can be detected in a subject. In some embodiments, a provided agent is administered before another agent is cleared out by a subject or vice versa. In some embodiments, a provided agent is administered within the half-life, or 2, 3, 4, 5 or 6 times of the half-life, of another agent or vice versa. In some embodiments, a subject is exposed to a therapeutic effect of a provided agent and a therapeutic effect of another therapeutic agent. In some embodiments, an agent may provide an effect after an agent is cleared out or metabolized by a subject. In some embodiments, a procedure, e.g., surgery, radiation, etc., may provide an effect after the procedure is completed.

In some embodiments, another therapy is a cancer therapy. In some embodiments, another therapy is or comprises surgery. In some embodiments, another therapy is or comprises radiation therapy. In some embodiments, another therapy is or comprises immunotherapy. In some embodiments, another therapeutic agent is or comprises a drug. In some embodiments, another therapeutic agent is or comprises a cancer drug. In some embodiments, another therapeutic agent is or comprises a chemotherapeutic agent. In some embodiments, another therapeutic agent is or comprises a hormone therapy agent. In some embodiments, another therapeutic agent is or comprises a kinase inhibitor. In some embodiments, another therapeutic agent is or comprises a checkpoint inhibitor (e.g., antibodies against PD-1, PD-L1, CTLA-4, etc.). In some embodiments, a provide agent can be administered with lower unit dose and/or total dose compared to being used alone. In some embodiments, another agent can be administered with lower unit dose and/or total dose compared to being used alone. In some embodiments, one or more side effects associated with administration of a provided agent and/or another therapy or therapeutic agent are reduced. In some embodiments, a combination therapy provides improved results, e.g., when compared to each agent utilized individually. In some embodiments, a combination therapy achieves one or more better results, e.g., when compared to each agent utilized individually.

In some embodiments, another agent is a checkpoint inhibitor, an EGFR inhibitor, a VEGF inhibitor, a VEGFR inhibitor, a kinase inhibitor, or an anti-cancer drug.

In some embodiments, an additional agent is a checkpoint inhibitor. In some embodiments, an additional agent is an immune oncology agent. In some embodiments, an additional agent is an antibody against a checkpoint molecules. In some embodiments, an additional agent is an antibody of PD1, PDL-1, CTLA4, A2AR, B7-H3, B7-H4, BTLA, IDO, KIR, LAG3, TIM-s, C₁₀orf54, etc. In some embodiments, an antibody is an anti-PD1 antibody. In some embodiments, an antibody is an anti-PD-L1 antibody. In some embodiments, an antibody is an anti-CTLA4.

In some embodiments, another agent is an EGFR inhibitor, e.g., erlotinib, gefitinib, lapatinib, panitumumab, vandetanib, cctuximab, etc. In some embodiments, another agent is an VEGF and/or VEGFR inhibitor, e.g., pazopanib, bevacizumab, sorafenib, sunitinib, axitinib, ponatinib, regorafenib, vandetanib, cabozantinib, ramucirumab, lenvatinib, ziv-aflibercept, etc. In some embodiments, another agent is a kinase inhibitor. In some embodiments, another therapeutic agent is a chemotherapeutic agent. In some embodiments, another therapeutic agent is an anti-cancer drug, e.g., cyclophosphamide, methotrexate, 5-fluorouracil (5-FU), doxorubicin, mustine, vincristine, procarbazine, prednisolone, dacarbazine, bleomycin, etoposide, cisplatin, epirubicin, capecitabine, folinic acid, actinomycin, all-trans retinoic acid, azacitidine, azathioprine, bortezomib, carboplatin, chlorambucil, cytarabine, daunorubicin, docetaxel, doxifluridine, fluorouracil, gemcitabine, hydroxyurea, idarubicin, imatinib, irinotecan, mechlorethamine, mercaptopurine, mitoxantrone, paclitaxel, pemetrexed, teniposide, tioguanine, topotecan, valrubicin, vinblastine, vindesine, vinorelbine, oxaliplatin, etc.

Among other things, the present disclosure provides the following Embodiments:

1. An agent having the structure of formula I:

or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is an amino acid residue that comprises a side chain comprising an acidic or polar group;
L^{AA2} is an amino acid residue that comprises a side chain comprising an acidic or polar group;
L^{AA3} is an amino acid residue;
L^{AA4} is an amino acid residue that comprises a side chain comprising an optionally substituted aromatic group;
L^{AA5} is an amino acid residue that comprises a side chain comprising an optionally substituted aromatic group;
L^{AA6} is an amino acid residue that comprises a side chain comprising an optionally substituted aromatic group;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C_{6 30} aryl, C_{6 30} arylaliphatic, C_{6 30} arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

2. An agent having the structure of formula I:

or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
   a first R′ group and a second R′ group which are taken together to form -L^{s}- which is bonded to the atom to which a first R′ group is attached and the atom to which a second R′ group is attached; and
   a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is L^{AR}, wherein a methylene unit is replaced with —C(R′)(R^{AS})—, wherein R^{AS} is -L^{AS1}-R^{AA1}, wherein R^{AA1} is —CO₂R or —SO₂R;
L^{AA2} is L^{AR}, wherein a methylene unit is replaced with —C(R′)(R^{AS})—, wherein R^{AS} is -L^{AS2}-R^{AA2}, wherein R^{AA2} is —CO₂R, or —SO₂R;
L^{AA3} is L^{AR}, wherein a methylene unit is replaced with —C(R′)(R^{AS})—, wherein R^{AS} is -L^{AS3}-R^{AA3}, wherein R^{AA3} is R′;
L^{AA4} is L^{AR}, wherein a methylene unit is replaced with —C(R′)(R^{AS})—, wherein R^{AS} is -L^{AS4}-R^{AA4}, wherein R^{AA4} is an optionally substituted group selected from 6-14 membered aryl or 5-14 membered heteroaryl having 1-6 heteroatoms;
L^{AA5} is L^{AR}, wherein a methylene unit is replaced with —C(R′)(R^{AS})—, wherein R^{AS} is -L^{AS5}-R^{AA5}, wherein R^{AA5} is an optionally substituted group selected from 6-14 membered aryl or 5-14 membered heteroaryl having 1-6 heteroatoms;
L^{AA6} is L^{AR}, wherein a methylene unit is replaced with —C(R′)(R^{AS})—, wherein R^{AS} is -L^{AS6}-R^{AA6}, wherein R^{AA6} is an optionally substituted group selected from 6-14 membered aryl or 5-14 membered heteroaryl having 1-6 heteroatoms;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L^{AR} is independently an optionally substituted, bivalent C₁-C₆ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each of L^{AS1}, L^{AS2}, L^{AS3}, L^{AS4}, L^{AS5}, and L^{AS6} is independently L^{AS};
each R^{AS} is independently -L^{AS}-R′;
each L^{AS} is independently a covalent bond or an optionally substituted, bivalent C₁-C₁₀ aliphatic or heteroaliphatic group having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

3. The agent of any one of the preceding Embodiments, wherein L^{P1} is or comprises -[X]ₚ-X¹-, wherein each X and X¹ is independently an amino acid residue, wherein p is 0-10, and X¹ is bonded to L^{AA1}.

4. The agent of any one of the preceding Embodiments, wherein L^{AA1} is an amino acid residue that comprises a side chain comprising an acidic group.

5. The agent of any one of the preceding Embodiments, wherein L^{P2} is or comprises -[X]pX³X⁴[X]p′-, wherein each X, X³ and X⁴ is independently an amino acid residue, and each of p and p′ is independently 0-10.

6. The agent of any one of the preceding Embodiments, wherein L^{AA2} is an amino acid residue that comprises a side chain comprising an acidic group.

7. The agent of any one of the preceding Embodiments, wherein L^{AA2} is X⁵.

8. The agent of any one of the preceding Embodiments, wherein L^{P3} is or comprises -[X]pX⁶X⁷[X]p′-, wherein each X, X⁶ and X⁷ is independently an amino acid residue, and each of p and p′ is independently 0-10.

9. The agent of any one of the preceding Embodiments, wherein L^{AA3} is an amino acid residue that comprises a side chain comprising an acidic group.

10. The agent of any one of the preceding Embodiments, wherein L^{P4} is or comprises -[X]pX⁷X⁸[X]p′-, wherein each X, X⁷ and X⁸ is independently an amino acid residue, and each of p and p′ is independently 0-10.

11. The agent of any one of the preceding Embodiments, wherein R^{AA4} is an optionally substituted group selected from phenyl, 5- or 6-membered monocyclic heteroaryl having 1-4 heteroatoms, and 9- or 10-membered bicyclic heteroaryl having 1-4 heteroatoms.

12. The agent of any one of the preceding Embodiments, wherein L^{P5} is or comprises [X]pX¹⁰X¹¹[X]p′-, wherein each X, X¹⁰ and X¹¹ is independently an amino acid residue, and each of p and p′ is independently 0-10.

13. The agent of any one of the preceding Embodiments, wherein R^{AA5} is an optionally substituted group selected from phenyl, 5- or 6-membered monocyclic heteroaryl having 1-4 heteroatoms, and 9- or 10-membered bicyclic heteroaryl having 1-4 heteroatoms.

14. The agent of any one of the preceding Embodiments, wherein the length of L^{P6} is a covalent bond.

15. The agent of any one of the preceding Embodiments, wherein R^{AA6} is an optionally substituted group selected from phenyl, 5- or 6-membered monocyclic heteroaryl having 1-4 heteroatoms, and 9- or 10-membered bicyclic heteroaryl having 1-4 heteroatoms.

16. The agent of any one of the preceding Embodiments, wherein L^{P7} is or comprises —X¹⁴—[X]p′-, wherein p′ is 0-10, each of X and X¹⁴ is independently an amino acid residue, and X¹⁴ is bonded to L^{AA6}.

17. The agent of any one of the preceding Embodiments, wherein R^{N} is R′, wherein R′ is —C(O)R, —CO₂R, or —SO₂R.

18. The agent of any one of the preceding Embodiments, wherein R^{C} is —OR′ or —N(R′)₂, wherein each R′ is independently R.

19. An agent comprising one or more of:
a first acidic group (e.g., of a first acidic amino acid residue);
a second acidic group (e.g., of a second acidic amino acid residue);
a first aromatic group (e.g., of a first aromatic amino acid residue);
a second aromatic group (e.g., of a first aromatic amino acid residue); and
a third aromatic group (e.g., of a third aromatic amino acid residue).

20. An agent comprising:
a first acidic group (e.g., of a first acidic amino acid residue);
a second acidic group (e.g., of a second acidic amino acid residue);
a first aromatic group (e.g., of a first aromatic amino acid residue);
a second aromatic group (e.g., of a first aromatic amino acid residue); and
a third aromatic group (e.g., of a third aromatic amino acid residue).

21. An agent comprising:
a first acidic group (e.g., of a first acidic amino acid residue);
a second acidic group (e.g., of a second acidic amino acid residue);
a third acidic group (e.g., of a third acidic amino acid residue);
a first aromatic group (e.g., of a first aromatic amino acid residue);
a second aromatic group (e.g., of a first aromatic amino acid residue); and
a third aromatic group (e.g., of a third aromatic amino acid residue).

22. An agent comprising:
a first acidic group (e.g., of a first acidic amino acid residue);
a second acidic group (e.g., of a second acidic amino acid residue);
a hydrophobic group (e.g., of a hydrophobic amino acid residue)
a first aromatic group (e.g., of a first aromatic amino acid residue);
a second aromatic group (e.g., of a first aromatic amino acid residue); and
a third aromatic group (e.g., of a third aromatic amino acid residue).

23. An agent comprising:
a first acidic group (e.g., of a first acidic amino acid residue);
a second acidic group (e.g., of a second acidic amino acid residue);
a third acidic group (e.g., of a third acidic amino acid residue);
a hydrophobic group (e.g., of a hydrophobic amino acid residue)
a first aromatic group (e.g., of a first aromatic amino acid residue);
a second aromatic group (e.g., of a first aromatic amino acid residue); and
a third aromatic group (e.g., of a third aromatic amino acid residue).

24. The agent of any one of the preceding Embodiments, wherein the distance between a first acidic group and a second acidic group is about the distance between the acidic groups of two acidic amino acid residues of a peptide motif, wherein there are two amino acid residues between the two acidic amino acid residues.

25. The agent of any one of the preceding Embodiments, wherein a first acidic amino acid residue is at position N and a second is at position N+3.

26. The agent of any one of the preceding Embodiments, wherein the distance between a first acidic group and a third acidic group is about the distance between the acidic groups of two acidic amino acid residues of a peptide motif, wherein there are three amino acid residues between the two acidic amino acid residues.

27. The agent of any one of the preceding Embodiments, wherein a first acidic amino acid residue is at position N and a third is at position N+4.

28. The agent of any one of the preceding Embodiments, wherein the distance between a first acidic group and a hydrophobic group is about the distance between the acidic group of an acidic amino acid residue and the hydrophobic group of a hydrophobic amino acid residue of a peptide motif, wherein there are five amino acid residues between the first acidic amino acid residue and the hydrophobic amino acid residue. 29. The agent of any one of the preceding Embodiments, wherein a first acidic amino acid residue is at position N and a hydrophobic amino acid residue is at position N+6.

30. The agent of any one of the preceding Embodiments, wherein the distance between a first acidic group and a first aromatic group is about the distance between the acidic group of a first acidic amino acid residue and the aromatic group of an aromatic amino acid residue of a peptide motif, wherein there are six amino acid residues between the first acidic amino acid residue and the first aromatic amino acid residue. 31. The agent of any one of the preceding Embodiments, wherein a first acidic amino acid residue is at position N and a first aromatic amino acid residue is at position N+7.

32. The agent of any one of the preceding Embodiments, wherein the distance between the first aromatic group and the second aromatic group is about the distance between the aromatic groups of two aromatic amino acid residues of a peptide motif, wherein there are two amino acid residues between the two aromatic amino acid residues.

33. The agent of any one of the preceding Embodiments, wherein a first aromatic amino acid residue is at position M and a second is at position M+3.

34. The agent of any one of the preceding Embodiments, wherein the distance between the first aromatic group and the third aromatic group is about the distance between the aromatic groups of two aromatic amino acid residues of a peptide motif, wherein there are three amino acid residues between the two aromatic amino acid residues.

35. The agent of any one of the preceding Embodiments,, wherein a first aromatic amino acid residue is at position N and a third is at position M+4).

36. The agent of any one of the preceding Embodiments, wherein N is 1, 2, 3, 4, or 5.

37. The agent of any one of the preceding Embodiments, wherein M is N+7.

38. The agent of any one of the preceding Embodiments, wherein M is 8-16.

39. The agent of any one of Embodiments 24-38, wherein the peptide motif is an alpha-helical motif wherein each amino acid residue is independently an alpha amino acid residue.

40. The agent of Embodiment 39, wherein the peptide motif is stapled.

41. The agent of Embodiment 40, wherein there are two staples in the peptide motif.

42. The agent of Embodiment 40, wherein there are three staples in the peptide motif.

43. The agent of any one of the preceding Embodiments, wherein the agent is or comprise a peptide.

44. The agent of any one of the preceding Embodiments, wherein the peptide is a stapled peptides comprising two or more staples.

45. The agent of any one of the preceding Embodiments, wherein the peptide is a stapled peptides comprising three or more staples.

46. The agent of any one of the preceding Embodiments, wherein the peptide is a stapled peptides comprising three and no more than three staples.

47. The agent of any one of the preceding Embodiments, wherein a first acidic group, a second acidic group, a third acidic group, a hydrophobic group, a first aromatic group, a second aromatic group and a third aromatic group, if present, are presented from N to C direction of a peptide.

48. The agent of any one of the preceding Embodiments, wherein the agent is or comprises a helix structure.

49. An agent, comprising:

wherein:
each of X¹, X², X³, X⁴, X⁵, X⁶, X⁷, X⁸, X⁹, X¹⁰, X¹¹, X¹², X¹³, and X¹⁴ is independently an amino acid residue, wherein:
X² comprises a side chain comprising an acidic or a polar group;
X⁵ comprises a side chain comprising an acidic or a polar group; and
each of X⁹, X¹² and X¹³ comprises a side chain comprising an optionally substituted aromatic group.

50. An agent, wherein the agent is or comprises a peptide comprising:

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled.

51. The agent of any one of the preceding Embodiments, the agent comprises three or more staples within 10-20 amino acid residues.

52. The agent of any one of the preceding Embodiments, wherein three or more of X⁰, X¹, X³, X⁴, X⁷, X¹⁰, X¹¹ and X¹⁴ are each independently an amino acid residue suitable for stapling, or are each independently stapled.

53. The agent of any one of the preceding Embodiments, wherein X¹ and X⁴ are each independently an amino acid residue suitable for stapling or are

54. The agent of any one of Embodiments 1-52, wherein X¹ and X⁴ are connected by a staple.

55. The agent of any one of the preceding Embodiments, wherein X⁴ and X¹¹ are each independently an amino acid residue suitable for stapling.

56. The agent of any one of Embodiments 1-54, wherein X⁴ and X¹¹ are connected by a staple.

57. The agent of any one of the preceding Embodiments, wherein X¹⁰ and X¹⁴ are each independently an amino acid residue suitable for stapling.

58. The agent of any one of Embodiments 1-56, wherein X¹⁰ and X¹⁴ are connected by a staple.

59. The agent of any one of the preceding Embodiments, wherein X⁷ and X¹⁰ are each independently an amino acid residue suitable for stapling.

60. The agent of any one of the preceding Embodiments, wherein the agent comprises a N-terminal group.

61. The agent of any one of the preceding Embodiments, wherein the N-terminal group is an acyl group.

62. The agent of Embodiment 60, wherein the N-terminal group is Ac.

63. The agent of any one of Embodiments 1-62, wherein X¹ is a residue of an amino acid that comprises —CH═CH₂.

64. The agent of any one of the preceding Embodiments, wherein X¹ is a residue of an amino acid that comprises —CH═CH₂ and forms a staple with another amino acid residue through olefin metathesis.

65. The agent of any one of Embodiments 1-62, wherein X¹ is PL3.

66. The agent of any one of the preceding Embodiments, wherein X⁴ is a residue of an amino acid that comprises an olefin.

67. The agent of any one of the preceding Embodiments, wherein X⁴ is a residue of an amino acid that comprises two —CH═CH₂.

68. The agent of any one of any one of the preceding Embodiments, wherein X⁴ is a residue of an amino acid that comprises two —CH═CH₂ and each forms a staple with another amino acid residue through olefin metathesis.

69. The agent of any one of the preceding Embodiments, wherein X⁴ is B5.

70. The agent of any one of the preceding Embodiments, wherein X¹⁰ is a residue of an amino acid that comprises an optionally substituted carboxyl group, an optionally substituted amino group, an azidyl group, an optionally substituted alkynyl group, or an optionally substituted thiol group.

71. The agent of any one of the preceding Embodiments, wherein X¹⁰ is Lys, GlnR, TriAzLys, sAla, dLys, AsnR, hGlnR, iPrLys, TriAzOrn, DGInR, Orn, 4PipA, sCH2S, [8FBB]Cys, [4FB]Cys, [mXyl]Cys, [oXyl]Cys, [pXyl]Cys, dOrn, dDab, NMeOrn, [2_6-naph]Cys, or [3_3-biph]Cys.

72. The agent of any one of the preceding Embodiments, wherein X¹⁰ is Lys.

73. The agent of any one of the preceding Embodiments, wherein X¹¹ is a residue of an amino acid that comprises —CH═CH₂.

74. The agent of any one of the preceding Embodiments, wherein X¹¹ is a residue of an amino acid that comprises —CH═CH₂ and forms a staple with another amino acid residue through olefin metathesis.

75. The agent of any one of the preceding Embodiments, wherein X¹¹ is PyrS2.

76. The agent of any one of the preceding Embodiments, wherein X¹⁴ is a residue of an amino acid that comprises a carboxyl group, an amino group, an azidyl group, an alkynyl group, or a thiol group.

77. The agent of any one of the preceding Embodiments, wherein X¹⁴ is GlnR, Lys, sAla, Gln, Cys, TriAzLys, AsnR, hGlnR, 4PipA, sAbu, Orn, GlnR, [4mampiperidine]GlnR, [39N2spiroundecane]GlnR, [29N2spiroundecane]GlnR, iPrLys, sCH2S, [diaminobutane]GlnR, [4aminopiperidine]GlnR, dGInR.

78. The agent of any one of the preceding Embodiments, wherein X¹⁴ is Glu.

79. The agent of any one of the preceding Embodiments, wherein X¹⁰ and X¹⁴ are connected by a staple, wherein the staple comprises —C(O)N(R′)—.

80. The agent of any one of the preceding Embodiments, wherein X² comprises a side chain comprising an acidic group.

81. The agent of any one of the preceding Embodiments, wherein X² comprises a side chain comprising —COOH or a salt form thereof.

82. The agent of any one of the preceding Embodiments, wherein X² is Asp.

83. The agent of any one of the preceding Embodiments, wherein X³ comprises one or two hydrophobic side chains.

84. The agent of any one of the preceding Embodiments, wherein the side chain of X³ is C₁₋₁₀ alkyl 85. The agent of any one of Embodiments 1-84, wherein X³ is Npg.

86. The agent of any one of Embodiments 1-84, wherein X³ is Ile.

87. The agent of any one of Embodiments 1-84, wherein X³ is Cha.

88. The agent of any one of Embodiments 1-84, wherein X³ is DipA.

89. The agent of any one of Embodiments 1-84, wherein X³ is Chg.

90. The agent of any one of Embodiments 1-84, wherein X³ is Leu.

91. The agent of any one of Embodiments 1-84, wherein X³ is Ala.

92. The agent of any one of the preceding Embodiments, wherein X⁵ comprises a side chain comprising an acidic group.

93. The agent of any one of the preceding Embodiments, wherein X⁵ comprises a side chain comprising —COOH or a salt form thereof.

94. The agent of any one of the preceding Embodiments, wherein X⁵ is Asp.

95. The agent of any one of Embodiments 1-93, wherein X⁵ is 3COOHF.

96. The agent of any one of Embodiments 1-93, wherein X⁵ is Glu.

97. The agent of any one of the preceding Embodiments, wherein X⁶ comprises a side chain comprising an acidic group.

98. The agent of any one of the preceding Embodiments, wherein X⁶ comprises a side chain comprising —COOH or a salt form thereof.

99. The agent of any one of the preceding Embodiments, wherein X⁶ is 3COOHF.

100. The agent of any one of Embodiments 1-98, wherein X⁶ is TfeGA.

101. The agent of any one of Embodiments 1-98, wherein X⁶ is Asp.

102. The agent of any one of Embodiments 1-98, wherein X⁶ is Glu.

103. The agent of any one of the preceding Embodiments, wherein X⁷ is a hydrophobic amino acid residue.

104. The agent of any one of the preceding Embodiments, wherein X⁷ is Aib.

105. The agent of any one of Embodiments 1-102, wherein X⁷ is Ala.

106. The agent of any one of Embodiments 1-102, wherein X⁷ is CyLeu.

107 The agent of any one of Embodiments 1-102, wherein X⁷ is Phe.

108. The agent of any one of Embodiments 1-102, wherein X⁷ is nLeu.

109. The agent of any one of Embodiments 1-102, wherein X⁷ is Val.

110. The agent of any one of the preceding Embodiments, wherein X⁸ is a hydrophobic amino acid residue.

111. The agent of any one of the preceding Embodiments, wherein X⁸ is Ala.

112. The agent of any one of Embodiments 1-109, wherein X⁸ is Aib.

113. The agent of any one of Embodiments 1-109, wherein X⁸ is Phe.

114. The agent of any one of Embodiments 1-109, wherein X⁸ is Asp.

115. The agent of any one of the preceding Embodiments, wherein X⁹ comprises a side chain which is or comprises an optionally substituted aromatic group.

116. The agent of any one of the preceding Embodiments, wherein X⁹ is Phe, 2Thi, Tyr, 3Thi, 4FF, 4CIF, 4BrF, 3FF, 3CIF, 3BrF, 2FF, 30MeF, 4CNF, 3CNF, 4MeF, 3MeF, BztA, 1NapA, Trp or 3F3MeF.

117. The agent of any one of the preceding Embodiments, wherein X⁹ is Phe.

118. The agent of any one of the preceding Embodiments, wherein X¹² is selected from 3Thi, 2F3McF, Phe, nLeu, 2COOHF, CypA, 2CIF, Ala, Abu, Leu, hLeu, Npg, Cpa, Nva, Cba, ChA, 2FurA, 20MeF, 2MeF, 2BrF, 2CNF, 2NO₂F, 2PyrA, 3PyrA, 4PyrA, His, 1NapA, Val, Ile, Chg, DiethA, hnLeu, OctG, 2Thi, and 2cbmF.

119. The agent of any one of the preceding Embodiments, wherein X¹² comprises a side chain which is or comprises an optionally substituted aromatic group.

120. The agent of any one of the preceding Embodiments, wherein X¹² is 3Thi.

121. The agent of any one of Embodiments 1-119, wherein X¹² is Phe.

122. The agent of any one of Embodiments 1-119, wherein X¹² is 2F3MeF.

123. The agent of any one of Embodiments 1-119, wherein X¹² is 2CIF.

124 The agent of any one of Embodiments 1-119, wherein X¹² is BztA.

125. The agent of any one of Embodiments 1-119, wherein X¹² is 2Thi.

126. The agent of any one of Embodiments 1-119, wherein X¹² is 2MeF.

127. The agent of any one of Embodiments 1-119, wherein X¹² is 2FF.

128. The agent of any one of Embodiments 1-119, wherein X¹² is 34CIF.

129. The agent any one of the preceding Embodiments, wherein the side chain of X¹³ comprises an optionally substituted aromatic group.

130. The agent of any one of the preceding Embodiments, wherein X¹³ is BtzA.

131. The agent of any one of Embodiments 1-129, wherein X¹³ is 2NapA.

132. The agent of any one of Embodiments 1-129, wherein X¹³ is 34CIF.

133. The agent of any one of Embodiments 1-129, wherein X¹³ is 3Thi.

134. The agent of any one of Embodiments 1-129, wherein X¹³ is Phe.

135. The agent of any one of Embodiments 1-129, wherein X¹³ is GlnR.

136. The agent of any one of Embodiments 1-129, wherein X¹³ is 34MeF.

137. The agent of any one of Embodiments 1-129, wherein X¹³ is 2NapA.

138. The agent of any one of the preceding Embodiments, wherein X¹³ interacts with Gln379 of beta-catenin or an amino acid residue corresponding thereto.

139. The agent of any one of the preceding Embodiments, wherein X¹³ interacts with Leu382 of beta-catenin or an amino acid residue corresponding thereto.

140. The agent of any one of the preceding Embodiments, wherein X¹³ interacts with Val416 of beta-catenin or an amino acid residue corresponding thereto.

141. The agent of any one of the preceding Embodiments, wherein X¹³ interacts with Asn415 of beta-catenin or an amino acid residue corresponding thereto.

142. The agent of any one of the preceding Embodiments, wherein X¹³ interacts with Trp383 of beta-catenin or an amino acid residue corresponding thereto.

143. The agent of any one of the preceding Embodiments, wherein p15 is 1.

144. The agent of any one of Embodiments 1-142, wherein p15 is 0.

145. The agent of any one of the preceding Embodiments, wherein p16 is 1.

146. The agent of any one of Embodiments 1-144, wherein p16 is 0.

147. The agent of any one of the preceding Embodiments, wherein p17 is 1.

148. The agent of any one of Embodiments 1-146, wherein p17 is 0.

149. The agent of any one of the preceding Embodiments, wherein p18 is 1.

150. The agent of any one of Embodiments 1-148, wherein p18 is 0.

151. The agent of any one of the preceding Embodiments, wherein p19 is 1.

152. The agent of any one of Embodiments 1-148, wherein p19 is 0.

153. The agent of any one of the preceding Embodiments, wherein p20 is 1.

154. The agent of any one of Embodiments 1-152, wherein p20 is 0.

155. The agent of any one of the preceding Embodiments, wherein p21 is 1.

156. The agent of any one of Embodiments 1-154, wherein p21 is 0.

157. The agent of any one of the preceding Embodiments, wherein p22 is 1.

158. The agent of any one of Embodiments 1-156, wherein p22 is 0.

159. The agent of any one of the preceding Embodiments, wherein p23 is 1.

160 The agent of any one of Embodiments 1-158, wherein p23 is 0.

161. The agent of any one of the preceding Embodiments, wherein the C-terminal group is R^{C}.

162. The agent of any one of the preceding Embodiments, wherein the C-terminal group comprises a PEG moiety.

163. The agent of any one of Embodiments 1-160, wherein the C-terminal group is —OH.

164. The agent of any one of Embodiments 1-160, wherein the C-terminal group is —O-L^{RC}-R′.

165. The agent of Embodiment 164, wherein L^{RC} is a covalent bond.

166. The agent of Embodiment 164, wherein L^{RC} is optionally substituted C₁₋₆ alkylene.

167. The agent of Embodiment 164, wherein L^{RC} is optionally substituted —CH₂—.

168. The agent of any one of Embodiments 164-167, wherein the R′ is optionally substituted C₁₋₁₀ aliphatic.

169. The agent of any one of Embodiments 164-167, wherein the R′ is optionally substituted C₁₋₁₀ alkyl.

170. The agent of any one of Embodiments 164-167, wherein the R′ is optionally substituted C₁₋₃₀ heteroaliphatic having 1-10 heteroatoms.

171. The agent of any one of Embodiments 164-167, wherein the R′ is optionally substituted C₁₋₃₀ heteroaliphatic having 1-10 oxygen atoms.

172. The agent of any one of Embodiments 164-167, wherein the R′ is optionally substituted C₆₋₃₀ aryl.

173. The agent of any one of Embodiments 164-167, wherein the R′ is optionally substituted 5-30 membered heteroaryl having 1-10 heteroatoms.

174. The agent of any one of Embodiments 1-160, wherein the C-terminal group is —N(R)₂.

175. The agent of any one of Embodiments 1-160, wherein the C-terminal group is —N(R)₂, wherein each R is independently —H or optionally substituted C₁₋₆ aliphatic.

176. The agent of any one of Embodiments 1-160, wherein the C-terminal group is —NH₂.

177. The agent of any one of Embodiments 1-160, wherein the C-terminal group is —NHMe.

178. The agent of any one of Embodiments 1-160, wherein the C-terminal group is —NHEt.

179. The agent of any one of Embodiments 1-160, wherein the C-terminal group is Serol.

180. The agent of any one of Embodiments 1-160, wherein the C-terminal group is dAlaol.

181. The agent of any one of the preceding Embodiments, wherein the peptide has the structure of:
R^{N}-[X]ₚ-[X°]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇—[X]p′-R^{C}, or a salt thereof, wherein:
each X is independently an amino acid residue;
each p and p′ is independently 0-10;
R^{N} is independently a peptide, an amino protecting group or R′-L^{RN}-;
R^{C} is independently a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

182. The agent of any one of the preceding Embodiments, wherein p is 0.

183. The agent of any one of the preceding Embodiments, wherein p is 1-10 (e.g., 1, 2, 3, 4, 5, 6, 7, 8, 9, or 10).

184. The agent of any one of the preceding Embodiments, wherein p′ is 0.

185. The agent of any one of Embodiments 1-183, wherein p′ is 1-10 (e.g., 1, 2, 3, 4, 5, 6, 7, 8, 9, or 10).

186. The agent of any one of the preceding Embodiments, wherein R^{N} is —C(O)R.

187. The agent of any one of the preceding Embodiments, wherein R^{N} is Ac.

188. The agent of any one of the preceding Embodiments, wherein R^{C} is —N(R′)₂.

189. The agent of any one of the preceding Embodiments, wherein R^{C} is —N(R)₂.

190. The agent of any one of Embodiments 1-189, wherein R^{C} is —NH₂.

191. The agent of any one of Embodiments 1-189, wherein R^{C} is —NHEt.

192. The agent of any one of Embodiments 1-189, wherein R^{C} is -Alaol, wherein the amino group of -Alaol is bonded to the last —C(O)— of the peptide backbone (R^{C} is

193. The agent of any one of Embodiments 1-189, wherein R^{C} is -dAlaol, wherein the amino group of -dAlaol is bonded to the last —C(O)— of the peptide backbone (R^{C} is

194. The agent of any one of Embodiments 1-189, wherein R^{C} is -Prool, wherein the amino group of -Prool is bonded to the last —C(O)— of the peptide backbone (R^{C} is

195. The agent of any one of Embodiments 1-189, wherein R^{C} is -Throl, wherein the amino group of -Throl is bonded to the last —C(O)— of the peptide backbone (R^{C} is

196. The agent of any one of Embodiments 1-189, wherein R^{C} is -Serol, wherein the amino group of -Serol is bonded to the last —C(O)— of the peptide backbone (R^{C} is

197. The agent of any one of Embodiments 1-187, wherein R^{C} is —O-L^{RC}-R′.

198. The agent of Embodiment 197, wherein L^{RC} is a covalent bond.

199. The agent of Embodiment 197, wherein L^{RC} is optionally substituted C₁₋₆ alkylene.

200. The agent of Embodiment 197, wherein L^{RC} is optionally substituted —CH₂—.

201. The agent of any one of Embodiments 197-200, wherein the R′ is optionally substituted C₁₋₁₀ aliphatic.

202. The agent of any one of Embodiments 197-200, wherein the R′ is optionally substituted C₁₋₁₀ alkyl.

203. The agent of any one of Embodiments 197-200, wherein the R′ is optionally substituted C₁₋₃₀ heteroaliphatic having 1-10 heteroatoms.

204. The agent of any one of Embodiments 197-200, wherein the R′ is optionally substituted C₁₋₃₀ heteroaliphatic having 1-10 oxygen atoms.

205. The agent of any one of Embodiments 197-200, wherein the R′ is optionally substituted C₆₋₃₀ aryl.

206. The agent of any one of Embodiments 197-200, wherein the R′ is optionally substituted 5-30 membered heteroaryl having 1-10 heteroatoms.

207. The agent of any one of Embodiments 1-187, wherein R^{C} is —OH.

208. The agent of any one of the preceding Embodiments, wherein the peptide forms a structure that comprises a helix.

209. The agent of any one of the preceding Embodiments, wherein the peptide binds to beta-catenin.

210. The agent of any one of the preceding Embodiments, wherein the peptide binds to a polypeptide whose sequence is or comprising SEQ ID NO: 2, or a fragment thereof:

(SEQ ID NO: 2)
SVLFYAITTLHNLLLHQEGAKMAVRLAGGLQKMVALLNKTNVKFL
AITTDCLQILAYGNQESKLIILASGGPQALVNIMRTYTYEKLLWT
TSRVLKVLSVCSSNKPAIVEAGGMQALGLHLTDPSQRLVQNCLWT
LRNLSDAATKQEGMEGLLGTLVQLLGSDDINVVTCAAGILSNLTC
NNYKNKMMVCQVGGIEALVRT

211. The agent of any one of the preceding Embodiments, wherein the agent interacts with Y306 of beta-catenin or an amino acid residue corresponding thereto.

212. The agent of any one of the preceding Embodiments, wherein the agent interacts with G307 of beta-catenin or an amino acid residue corresponding thereto.

213. The agent of any one of the preceding Embodiments, wherein the agent interacts with K312 of beta-catenin or an amino acid residue corresponding thereto.

214. The agent of any one of the preceding Embodiments, wherein the agent interacts with K345 of beta-catenin or an amino acid residue corresponding thereto.

215. The agent of any one of the preceding Embodiments, wherein the agent interacts with Q379 of beta-catenin or an amino acid residue corresponding thereto.

216. The agent of any one of the preceding Embodiments, wherein the agent interacts with L382 of beta-catenin or an amino acid residue corresponding thereto.

217. The agent of any one of the preceding Embodiments, wherein the agent interacts with W383 of beta-catenin or an amino acid residue corresponding thereto.

218. The agent of any one of the preceding Embodiments, wherein the agent interacts with N387 of beta-catenin or an amino acid residue corresponding thereto.

219. The agent of any one of the preceding Embodiments, wherein the agent interacts with D413 of beta-catenin or an amino acid residue corresponding thereto.

220. The agent of any one of the preceding Embodiments, wherein the agent interacts with N415 of beta-catenin or an amino acid residue corresponding thereto.

221. The agent of any one of the preceding Embodiments, wherein the agent interacts with V416 of beta-catenin or an amino acid residue corresponding thereto.

222. An agent having a structure selected from Table E2 or a salt thereof.

223. An agent having a structure selected from Table E3 or a salt thereof.

224. An agent, having the structure of or a salt thereof.

225. An agent, having the structure of

or a salt thereof.

226. An agent, having the structure of

or a salt thereof.

227. An agent, having the structure of

or a salt thereof.

228. An agent, having the structure of

or a salt thereof.

229. An agent, having the structure of

or a salt thereof.

230. An agent, having the structure of

or a salt thereof.

231. An agent, having the structure of

or a salt thereof.

232. An agent, having the structure of

or a salt thereof.

233. An agent, having the structure of

or a salt thereof.

234. An agent, having the structure of

or a salt thereof.

235. An agent, having the structure of

or a salt thereof.

236. An agent, having the structure of

or a salt thereof.

237. An agent, having the structure of

or a salt thereof.

238. An agent, having the structure of

or a salt thereof.

239. The agent of any one of the preceding Embodiments, wherein a double bond of a staple bonded to the first stapled amino acid that is bonded to a staple with a double bond, counting from the N-terminus, is E.

240. The agent of any one of the preceding Embodiments, wherein a double bond of a staple bonded to the first stapled amino acid that is bonded to a staple with a double bond, counting from the N-terminus, is Z.

241. The agent of any one of the preceding Embodiments, wherein a double bond of a staple bonded to the first stapled amino acid that is bonded to a staple with a double bond, counting from the C-terminus, is E.

242. The agent of any one of the preceding Embodiments, wherein a double bond of a staple bonded to the first stapled amino acid that is bonded to a staple with a double bond, counting from the C-terminus, is Z.

243. The agent of any one of the preceding Embodiments, wherein a double bond of a (i, i+7) staple is E.

244. The agent of any one of the preceding Embodiments, wherein a double bond of a (i, i+7) staple is Z.

245. The agent of any one of the preceding Embodiments, wherein a double bond of a (i, i+2), (i, i+3) or (i, i+4) staple is E.

246. The agent of any one of the preceding Embodiments, wherein a double bond of a (i, i+2), (i, i+3) or (i, i+4) staple is Z.

247. The agent of any one of Embodiments 222-238, wherein a double bond of a staple bonded to the first amino acid from the N-terminus is Z.

248. The agent of any one of Embodiments 222-246, wherein a double bond of a staple bonded to the 11th amino acid from the N-terminus is E.

249. The agent of any one of Embodiments 222-246, wherein a double bond of a staple bonded to the 11th amino acid from the N-terminus is Z.

250. The agent of any one of the preceding Embodiments, wherein a carbon atom bonded to two staples (e.g., in B5) is of R configuration.

251. The agent of any one of any one of the preceding Embodiments, wherein a carbon atom bonded to two staples (e.g., in B5) is of S configuration.

252. An agent, having the structure of SP-1-1 or a salt thereof.

253. An agent, having the structure of SP-1-2 or a salt thereof.

254. An agent, having the structure of SP-1-3 or a salt thereof.

255. An agent, having the structure of SP-1-4 or a salt thereof.

256. An agent, having the structure of SP-1-5 or a salt thereof.

257. An agent, having the structure of SP-1-6 or a salt thereof.

258. An agent, having the structure of SP-1-7 or a salt thereof.

259. An agent, having the structure of SP-1-8 or a salt thereof.

260. An agent, having the structure of SP-2-1 or a salt thereof.

261. An agent, having the structure of SP-2-2 or a salt thereof.

262. An agent, having the structure of SP-2-3 or a salt thereof.

263. An agent, having the structure of SP-2-4 or a salt thereof.

264. An agent, having the structure of SP-2-5 or a salt thereof.

265. An agent, having the structure of SP-2-6 or a salt thereof.

266. An agent, having the structure of SP-2-7 or a salt thereof.

267. An agent, having the structure of SP-2-8 or a salt thereof.

268. An agent, having the structure of SP-3-1 or a salt thereof.

269. An agent, having the structure of SP-3-2 or a salt thereof.

270. An agent, having the structure of SP-4-1 or a salt thereof.

271. An agent, having the structure of SP-4-2 or a salt thereof.

272. An agent, having the structure of SP-4-3 or a salt thereof.

273. An agent, having the structure of SP-4-4 or a salt thereof.

274. An agent, having the structure of SP-4-5 or a salt thereof.

275. An agent, having the structure of SP-4-6 or a salt thereof.

276. An agent, having the structure of SP-4-7 or a salt thereof.

277. An agent, having the structure of SP-4-8 or a salt thereof.

278. An agent, having the structure of SP-5-1 or a salt thereof.

279. An agent, having the structure of SP-5-2 or a salt thereof.

280. An agent, having the structure of SP-5-3 or a salt thereof.

281. An agent, having the structure of SP-5-4 or a salt thereof.

282. An agent, having the structure of SP-5-5 or a salt thereof.

283. An agent, having the structure of SP-5-6 or a salt thereof.

284. An agent, having the structure of SP-5-7 or a salt thereof.

285. An agent, having the structure of SP-5-8 or a salt thereof.

286. An agent, having the structure of SP-6 or a salt thereof.

287. An agent, having the structure of SP-7-1 or a salt thereof.

288. An agent, having the structure of SP-7-2 or a salt thereof.

289. An agent, having the structure of SP-7-3 or a salt thereof.

290. An agent, having the structure of SP-7-4 or a salt thereof.

291. An agent, having the structure of SP-7-5 or a salt thereof.

292. An agent, having the structure of SP-7-6 or a salt thereof.

293. An agent, having the structure of SP-7-7 or a salt thereof.

294. An agent, having the structure of SP-7-8 or a salt thereof.

295. An agent, having the structure of SP-8-1 or a salt thereof.

296. An agent, having the structure of SP-8-2 or a salt thereof.

297. An agent, having the structure of SP-8-3 or a salt thereof.

298. An agent, having the structure of SP-8-4 or a salt thereof.

299. An agent, having the structure of SP-8-5 or a salt thereof.

300. An agent, having the structure of SP-8-6 or a salt thereof.

301. An agent, having the structure of SP-8-7 or a salt thereof.

302. An agent, having the structure of SP-8-8 or a salt thereof.

303. An agent, having the structure of SP-9-1 or a salt thereof.

304. An agent, having the structure of SP-9-2 or a salt thereof.

305. An agent, having the structure of SP-9-3 or a salt thereof.

306. An agent, having the structure of SP-9-4 or a salt thereof.

307. An agent, having the structure of SP-9-5 or a salt thereof.

308. An agent, having the structure of SP-9-6 or a salt thereof.

309. An agent, having the structure of SP-9-7 or a salt thereof.

310. An agent, having the structure of SP-9-8 or a salt thereof.

311. An agent, having the structure of SP-10-1 or a salt thereof.

312. An agent, having the structure of SP-10-2 or a salt thereof.

313. An agent, having the structure of SP-10-3 or a salt thereof.

314. An agent, having the structure of SP-10-4 or a salt thereof.

315. An agent, having the structure of SP-10-5 or a salt thereof.

316. An agent, having the structure of SP-10-6 or a salt thereof.

317. An agent, having the structure of SP-10-7 or a salt thereof.

318. An agent, having the structure of SP-10-8 or a salt thereof.

319. An agent, having the structure of SP-11-1 or a salt thereof.

320. An agent, having the structure of SP-11-2 or a salt thereof.

321. An agent, having the structure of SP-11-3 or a salt thereof.

322. An agent, having the structure of SP-11-4 or a salt thereof.

323. An agent, having the structure of SP-11-5 or a salt thereof.

324. An agent, having the structure of SP-11-6 or a salt thereof.

325. An agent, having the structure of SP-11-7 or a salt thereof.

326. An agent, having the structure of SP-11-8 or a salt thereof.

327. An agent, having the structure of SP-12-1 or a salt thereof.

328. An agent, having the structure of SP-12-2 or a salt thereof.

329. An agent, having the structure of SP-12-3 or a salt thereof.

330. An agent, having the structure of SP-12-4 or a salt thereof.

331. An agent, having the structure of SP-12-5 or a salt thereof.

332. An agent, having the structure of SP-12-6 or a salt thereof.

333. An agent, having the structure of SP-12-7 or a salt thereof.

334. An agent, having the structure of SP-12-8 or a salt thereof.

335. An agent, having the structure of SP-13-1 or a salt thereof.

336. An agent, having the structure of SP-13-2 or a salt thereof.

337. An agent, having the structure of SP-13-3 or a salt thereof.

338. An agent, having the structure of SP-13-4 or a salt thereof.

339. An agent, having the structure of SP-13-5 or a salt thereof.

340. An agent, having the structure of SP-13-6 or a salt thereof.

341. An agent, having the structure of SP-13-7 or a salt thereof.

342. An agent, having the structure of SP-13-8 or a salt thereof.

343. An agent, having the structure of SP-14-1 or a salt thereof.

344. An agent, having the structure of SP-14-2 or a salt thereof.

345. An agent, having the structure of SP-14-3 or a salt thereof.

346. An agent, having the structure of SP-14-4 or a salt thereof.

347. An agent, having the structure of SP-14-5 or a salt thereof.

348. An agent, having the structure of SP-14-6 or a salt thereof.

349. An agent, having the structure of SP-14-7 or a salt thereof.

350. An agent, having the structure of SP-14-8 or a salt thereof.

351. An agent, having the structure of SP-15-1 or a salt thereof.

352. An agent, having the structure of SP-15-2 or a salt thereof.

353. An agent, having the structure of SP-15-3 or a salt thereof.

354. An agent, having the structure of SP-15-4 or a salt thereof.

355. An agent, having the structure of SP-15-5 or a salt thereof.

356. An agent, having the structure of SP-15-6 or a salt thereof.

357. An agent, having the structure of SP-15-7 or a salt thereof.

358. An agent, having the structure of SP-15-8 or a salt thereof.

359. An agent having the structure of

or a salt thereof.

360. An agent having the structure of

or a salt thereof.

361. An agent having the structure of

or a salt thereof.

362. The agent of any one of Embodiments 359-361, wherein the agent has the same retention time under a HPLC condition as I-66 prepared as described in Example 3, wherein the HPLC condition can separate I-66 and I-67 prepared as described in Example 3.

363. The agent of any one of Embodiments 359-361, wherein the agent shows a retention time of about 15.3 min under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

364. The agent of any one of Embodiments 359-363, wherein the agent elutes in a single peak with I-66 prepared as described in Example 3 under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

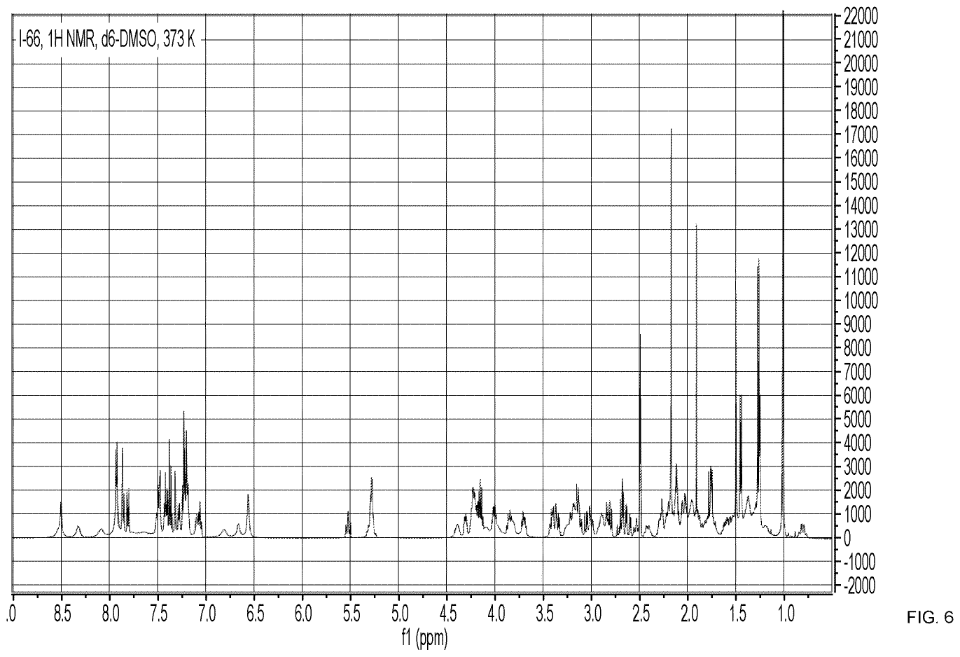

365. The agent of any one of Embodiments 359-364, characterized in that the agent shows ¹H NMR peaks that overlap with the peaks between about 5.1-5.7 in FIG. 6 under the same or comparable conditions. 366. The agent of any one of Embodiments 359-364, characterized in that the agent shows the same 1H NMR peaks between about 5.1-5.7 as FIG. 6 under the same or comparable conditions.

367. The agent of any one of Embodiments 359-364, characterized in that in its ¹H NMR spectrum, the peaks corresponding to 1H bonded to carbon atoms overlap with peaks in FIG. 6 under the same or comparable conditions.

368. The agent of any one of Embodiments 359-364, characterized in that its ¹H NMR spectrum overlaps with peaks in FIG. 6 under the same or comparable conditions.

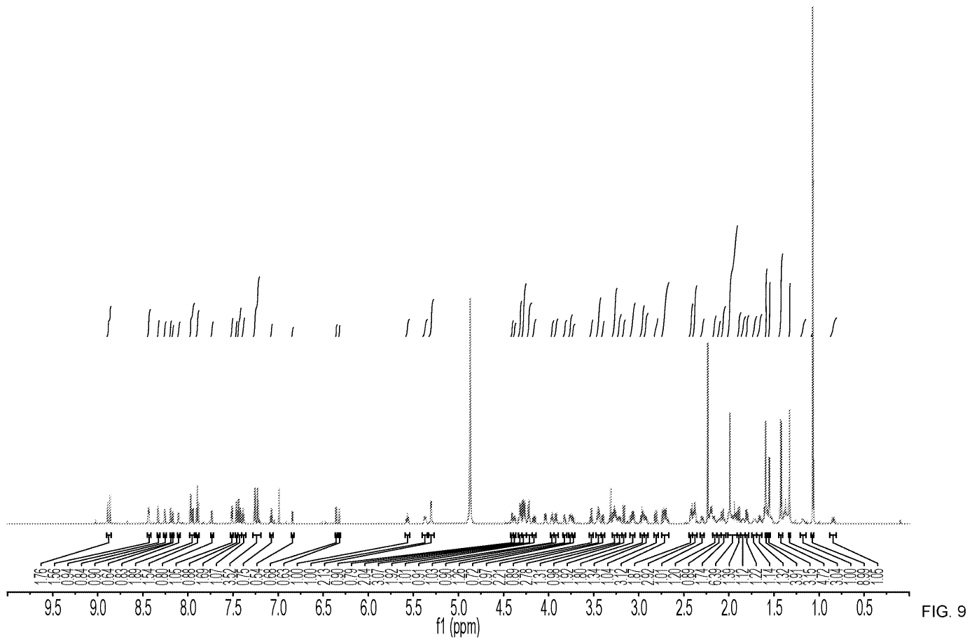

369. The agent of any one of Embodiments 359-364, characterized in that the agent shows ¹H NMR peaks that overlap with the peaks between about 5.1-5.7 in FIG. 9 under the same or comparable conditions.

370. The agent of any one of Embodiments 359-364, characterized in that the agent shows the same 1H NMR peaks between about 5.1-5.7 as FIG. 9 under the same or comparable conditions.

371. The agent of any one of Embodiments 359-364, characterized in that in its ¹H NMR spectrum, the peaks corresponding to 1H bonded to carbon atoms overlap with peaks in FIG. 9 under the same or comparable conditions.

372. The agent of any one of Embodiments 359-364, characterized in that its ¹H NMR spectrum overlaps with peaks in FIG. 9 under the same or comparable conditions.

373. The agent of any one of Embodiments 359-364, characterized in that its ¹H NMR spectrum comprises the following peaks (methanol-d4, about 298K, about 900 MHz, chemical shift): 3.96, 6.34, 6.84, 6.36, 4.38, 4.31, 3.82, 4.21, 7.21, 7.24, 7.07, 7.24, 7.21, 4.03, 4.25, 7.97, 7.94, 7.42, 7.50, 4.40, 5.29, 5.31, 4.27, 4.31, 5.56, 5.36, 7.89, 7.38, 7.41, 7.89, 7.00, 4.30, 2.00, 2.10, 1.60, 1.65, 3.73, 4.15, 3.15, 4.22, 2.29, 2.41, 3.53, 3.25, 3.06, 2.95, 1.93, 2.38, 2.92, 3.45, 1.18, 1.36, 1.55, 1.84, 1.53, 3.27, 3.31, 3.27, 3.44, 2.74, 2.96, 1.92, 2.07, 1.37, 2.38, 1.80, 1.88, 2.71, 2.81, 3.20, 2.05, 1.97, 2.17, 0.84, 1.42, 1.57, 2.69, 3.76, 3.92, 2.20, 2.42, 2.22, 3.08, 3.40, 1.55, 1.07, 1.42, 1.59, 1.33, and 2.23.

374. The agent of any one of Embodiments 359-364, characterized in that chemical shifts of ¹H bonded to carbon of the agent are (methanol-d4, about 298K, about 900 MHz): 3.96, 6.34, 6.84, 6.36, 4.38, 4.31, 3.82, 4.21, 7.21, 7.24, 7.07, 7.24, 7.21, 4.03, 4.25, 7.97, 7.94, 7.42, 7.50, 4.40, 5.29, 5.31, 4.27, 4.31, 5.56, 5.36, 7.89, 7.38, 7.41, 7.89, 7.00, 4.30, 2.00, 2.10, 1.60, 1.65, 3.73, 4.15, 3.15, 4.22, 2.29, 2.41, 3.53, 3.25, 3.06, 2.95, 1.93, 2.38, 2.92, 3.45, 1.18, 1.36, 1.55, 1.84, 1.53, 3.27, 3.31, 3.27, 3.44, 2.74, 2.96, 1.92, 2.07, 1.37, 2.38, 1.80, 1.88, 2.71, 2.81, 3.20, 2.05, 1.97, 2.17, 0.84, 1.42, 1.57, 2.69, 3.76, 3.92, 2.20, 2.42, 2.22, 3.08, 3.40, 1.55, 1.07, 1.42, 1.59, 1.33, and 2.23.

375. The agent of any one of Embodiments 359-364 and 373-374, characterized in that ¹H NMR of the agent comprises the following peaks, or peaks corresponding to the following peaks (methanol-d4, about 298K, about 900 MHz, chemical shift): 8.32, 7.73, 8.17, 8.19, 8.25, 8.44, 8.89, 8.10, 8.42, 7.45, 7.39, 8.15, 8.86, and 7.69.

376. The agent of any one of Embodiments 359-364 and 373-375, characterized in that ¹³C NMR of the agent comprises the following peaks (methanol-d4, about 298K, about 226 MHz, chemical shift): 65.89, 137.36, 138.13, 57.28, 138.06, 132.12, 169.51, 62.78, 31.36, 70.51, 132.57, 139.58, 141.98, 156.51, 173.13, 175.55, 175.8, 174.47, 175.8, 177.56, 178.58, 174.39, 175.7, 177.0, 175.8, 175.44, 175.6, 176.97, 175.55, 178.08, 172.38, 59.94, 123.42, 125.19, 129.15, 56.93, 55.69, 57.70, 60.70, 130.39, 129.83, 128.08, 129.83, 130.39, 54.54, 60.94, 131.96, 129.37, 129.54, 135.08, 55.56, 131.26, 131.47, 56.48, 55.33, 129.2, 126.96, 122.71, 125.37, 125.20, 123.87, 126.99, 51.20, 29.99, 30.63, 67.18, 53.94, 31.81, 45.37, 32.60, 27.47, 33.84, 39.25, 23.2, 35.8, 29.61, 38.18, 38.10, 36.18, 30.00, 23.32, 33.8, 45.96, 35.69, 33.09, 27.46, 23.29, 35.96, 49.50, 24.46, 36.46, 31.76, 17.00, 30.18, 17.76, 28.98, 23.20, 23.87, 172.48, and 174.10.

377. The agent of any one of Embodiments 359-364 and 373-375, characterized in that chemical shifts of ¹³C of the agent are (methanol-d4, about 298K, about 226 MHz, chemical shift): 65.89, 137.36, 138.13, 57.28, 138.06, 132.12, 169.51, 62.78, 31.36, 70.51, 132.57, 139.58, 141.98, 156.51, 173.13, 175.55, 175.8, 174.47, 175.8, 177.56, 178.58, 174.39, 175.7, 177.0, 175.8, 175.44, 175.6, 176.97, 175.55, 178.08, 172.38, 59.94, 123.42, 125.19, 129.15, 56.93, 55.69, 57.70, 60.70, 130.39, 129.83, 128.08, 129.83, 130.39, 54.54, 60.94, 131.96, 129.37, 129.54, 135.08, 55.56, 131.26, 131.47, 56.48, 55.33, 129.2, 126.96, 122.71, 125.37, 125.20, 123.87, 126.99, 51.20, 29.99, 30.63, 67.18, 53.94, 31.81, 45.37, 32.60, 27.47, 33.84, 39.25, 23.2, 35.8, 29.61, 38.18, 38.10, 36.18, 30.00, 23.32, 33.8, 45.96, 35.69, 33.09, 27.46, 23.29, 35.96, 49.50, 24.46, 36.46, 31.76, 17.00, 30.18, 17.76, 28.98, 23.20, 23.87, 172.48, and 174.10.

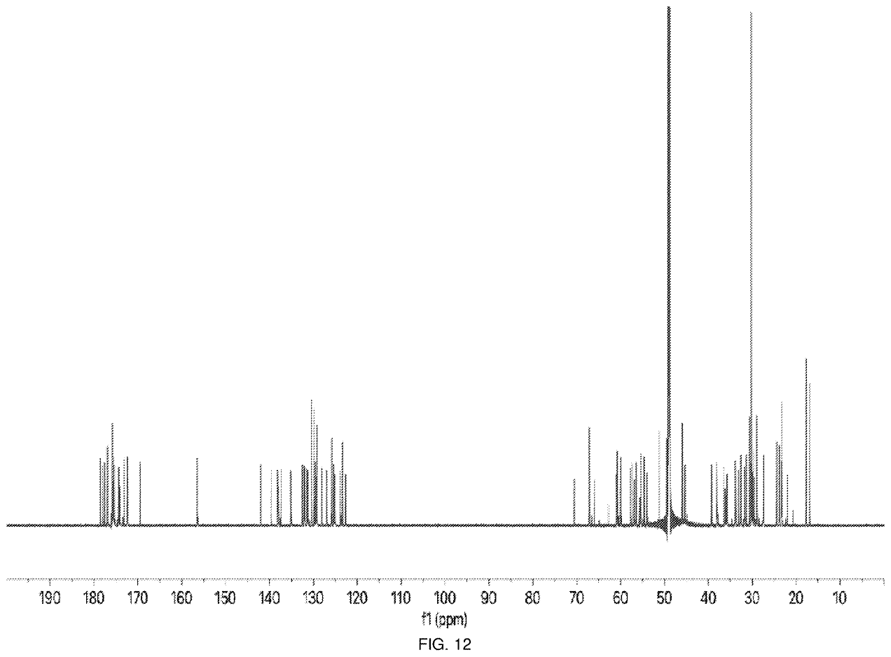

378. The agent of any one of Embodiments 359-364 and 373-377, characterized in that its ¹³C NMR spectrum overlaps with peaks in FIG. 12 under the same or comparable conditions.

379. The agent of any one of Embodiments 359-364, characterized in that its NMR spectra are the same or of the same compound as one or more or all of FIG. 9 to FIG. 18 under the same or comparable conditions.

380. The agent of any one of Embodiments 359-364, characterized in that its NMR spectra are the same or of the same compound as all of FIG. 9 to FIG. 18 under the same or comparable conditions.

381. An agent having the structure of

or a salt thereof.

382. The agent of any one of Embodiments 359-361, wherein the agent has the same retention time under a HPLC condition as I-67 prepared as described in Example 3, wherein the HPLC condition can separate I-66 and I-67 prepared as described in Example 3.

383. The agent of any one of Embodiments 359-361, wherein the agent shows a retention time of about 16.2 min under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

384. The agent of any one of Embodiments 359-361, wherein the agent elutes in a single peak with I-67 prepared as described in Example 3 under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

385. The agent of any one of Embodiments 359-361 and 382-384, characterized in that the agent shows 1H NMR peaks that do not overlap with the peaks between about 5.1-5.7 in FIG. 6 under the same or comparable conditions.

386. The agent of any one of Embodiments 359-361 and 382-384, characterized in that the agent does not show the same ¹H NMR peaks between about 5.1-5.7 as FIG. 6 under the same or comparable conditions.

387. The agent of any one of Embodiments 359-361 and 382-384, characterized in that in its ¹H NMR spectrum, the peaks corresponding to ¹H bonded to carbon atoms do not all overlap with peaks in FIG. 6 under the same or comparable conditions.

388. The agent of any one of Embodiments 359-361, characterized in that its ¹H NMR spectrum does not overlap with peaks in FIG. 6 under the same or comparable conditions.

389. The agent of any one of the preceding Embodiments, wherein each olefin double bond in a staple is converted into a single bond.

390. The agent of any one of the preceding Embodiments, wherein each olefin double bond is converted into a single bond.

391. The agent of any one of the preceding Embodiments, wherein each olefin double bond is independently and optionally converted into —CHR′—CHR′—, wherein each R is independently —H, -R, —OR, —OH, —N(R)₂, or —SR.

392. The agent of any one of the preceding Embodiments, wherein each olefin double bond is independently converted into optionally substituted —CH₂—CH₂—.

393. An agent of any one of Embodiments 1-1900 and 2073-2089 of WO 2022/261257.

394. The agent of any one of the preceding Embodiments, having a diastereopurity of about 80%, 85%, 90%, 91%, 92%, 93%, 94%, 95%, 96%, 97%, 98%, 99% or more.

395. The agent of any one of the preceding Embodiments, having a purity of about 80%, 85%, 90%, 91%, 92%, 93%, 94%, 95%, 96%, 97%, 98%, 99% or more.

396. The agent of any one of the preceding Embodiments, having a purity of about 90% or more. 397. The agent of any one of the preceding Embodiments, having a purity of about 95% or more.

398. The agent of any one of the preceding Embodiments, having a purity of about 98% or more.

399. A composition comprising an agent of any one of the preceding Embodiments or a salt thereof.

400. A pharmaceutical composition, comprising or delivering an agent of any one of the preceding Embodiments, and a pharmaceutically acceptable carrier.

401. A composition selected from Table E2.

402. A pharmaceutical composition, comprising or delivering one or more or all peptide agents in a composition selected from Table E2 and a pharmaceutically acceptable carrier.

403. A composition selected from Table E3.

404. A pharmaceutical composition, comprising or delivering one or more or all peptide agents in a composition selected from Table E3 and a pharmaceutically acceptable carrier.

405. The composition of any one of the preceding Embodiments, comprising an agent comprising one or more staples each independently comprises one or more olefin double bond.

406. The composition of any one of the preceding Embodiments, wherein each ratio of the two stereoisomers of each olefin double bond in each staple is independently about 2:1, 3:1, 4:1, 5:1, 6:1, 7:1, 8:1, 9:1, 10:1, 15:1, 20:1, 30:1, 40:1, 50:1 or more.

407. The composition of any one of the preceding Embodiments, wherein a selectivity is favoring an E configuration.

408. The composition of any one of the preceding Embodiments, wherein a selectivity is favoring a Z configuration.

409. A composition of any one of Embodiments 2090-2108 of WO 2022/261257.

410. The composition of any one of the preceding Embodiments, characterized in that characterized in that when the composition is administered to a hepatocellular cell, proliferation of the cell is reduced.

411. The composition of Embodiment 410, wherein the hepatocellular cancer cell comprises a mutation in beta-catenin.

412. The composition of any one of the preceding Embodiments, characterized in that characterized in that when the composition is administered to a hepatocellular tumor, growth of the tumor is reduced.

413. The composition of Embodiment 412, wherein the hepatocellular tumor comprises hepatocellular cancer cells comprising a mutation in beta-catenin.

414. The composition of Embodiment 411 or 413, wherein the mutation is D32H.

415. The composition of Embodiment 411 or 413, wherein the mutation is D32V.

416. The composition of Embodiment 411 or 413, wherein the mutation is S37C.

417. The composition of Embodiment 411 or 413, wherein the mutation is S45F.

418. The compositions of any one of Embodiments 410-417, wherein the reduction is compared to a reference.

419. The composition of Embodiment 418, wherein the reference is absence of administration of the agent.

420. The composition of Embodiment 418, wherein the reference is administration of a reference agent.

421. The composition of Embodiment 418, wherein the reference is administration of I-470.

422. The composition of any one of Embodiments 412-421, wherein the tumor is in a mouse PDX model.

423. The composition of Embodiment 422, wherein one or each dose administered to a mouse is independently about 20-80 mg/kg.

424. The composition of Embodiment 422, wherein one or each dose administered to a mouse is independently about 60 mg/kg.

425. The composition of any one of Embodiments 410-424, wherein reduction is observed after about 1, 2, 3, 4, 5, 6, 7, 8, 9, or 10 doses.

426. A method for modulating beta-catenin interaction with a partner in a system, comprising contacting beta-catenin with an agent or composition of any one of the preceding Embodiments.

427. A method for modulating beta-catenin interaction with a partner in a system, comprising administering or delivering to the system an agent or composition of any one of the preceding Embodiments.

428. The method of nay one of Embodiments 426-427, wherein the partner is TCF7, LEF1, TCF7L1, TCF7L2, Axin1, Axin2, or APC.

429. A method for modulating a TCF-beta-catenin interaction in a system, comprising contacting beta-catenin with an agent or composition of any one of the preceding Embodiments.

430. A method for modulating a TCF-beta-catenin interaction in a system, comprising administering or delivering to the system an agent or composition of any one of the preceding Embodiments.

431. A method for inhibiting beta-catenin dependent cell proliferation, comprising administering or delivering to the system an agent or composition of any one of the preceding Embodiments.

432. A method for modulating WNT/beta-catenin pathway in a system, comprising administering or delivering to the system an agent or composition of any one of the preceding Embodiments, wherein expression of a nucleic acid is modulated.

433. A method, comprising administering or delivering to the system an agent or composition of any one of the preceding Embodiments, wherein level of a transcript of a nucleic acid and/or a product thereof is modulated.

434. A method, comprising administering or delivering to the system an agent or composition of any one of the preceding Embodiments, wherein expression of a nucleic acid is modulated.

435. A method for reducing hepatocellular cancer cell proliferation in a system, comprising administering or delivering to the hepatocellular cancer cell an effective amount of an agent or composition of any one of the preceding Embodiments.

436. A method for reducing hepatocellular tumor growth in a system, comprising administering or delivering to the tumor an effective amount of an agent or composition of any one of the preceding Embodiments.

437. The method of any one of Embodiments 435-436, wherein the reduction is compared to a reference.

438. The composition of Embodiment 437, wherein the reference is absence of administration of the agent.

439. The composition of Embodiment 437, wherein the reference is administration of a reference agent.

440. The composition of Embodiment 437, wherein the reference is administration of I-470.

441. The method of any one of Embodiments 426-440, wherein a system is an in vitro system.

442. The method of any one of Embodiments 426-440, wherein a system is an in vivo system.

443. The method of any one of Embodiments 426-440, wherein a system is or comprises a sample.

444. The method of any one of Embodiments 426-443, wherein a system is or comprises a cell, tissue or organ.

445. The method of any one of Embodiments 426-444, wherein a system is or comprises cancer cells.

446. The method of any one of Embodiments 426-445, wherein a system is or comprises a tumor.

447. The method of any one of Embodiments 426-446, wherein a system is a subject.

448. The method of any one of Embodiments 426-447, wherein a system comprises hepatocellular cancer cells.

449. The method of any one of Embodiments 435-440 and 448, wherein the hepatocellular cancer cells comprise a mutation in beta-catenin.

450. A method for treating hepatocellular cancer in a subject, comprising administering or delivering to the subject an effective amount of an agent or composition of any one of the preceding Embodiments.

451. The method of Embodiment 450, wherein the subject comprises hepatocellular cancer cells that comprise a mutation in beta-catenin.

452. The method of Embodiment 449 or 451, wherein the mutation is D32H.

453. The method of Embodiment 449 or 451, wherein the mutation is D32H.

454. The method of Embodiment 449 or 451, wherein the mutation is S37C.

455. The method of Embodiment 449 or 451, wherein the mutation is S45F.

456. The method of any one of the preceding Embodiments, comprising administering or deliver to a system or subject a second therapeutic agent.

457. The method of any one of the preceding Embodiments, comprising administering or deliver to a system or subject a second therapy.

458. The method of Embodiment 456 or 457, wherein a second therapeutic agent or therapy is administered prior to an agent of any one of the preceding Embodiments.

459. The method of Embodiment 456 or 457, wherein a second therapeutic agent or therapy is administered about or no more than about 1, 2, 3, 4, 5, 6, or 7 days, or 1, 2, 3, or weeks, or 1, 2, 3, 4, 5, or 6 months, prior to an agent of any one of the preceding Embodiments.

460. The method of Embodiment 456 or 457, wherein a second therapeutic agent or therapy is administered concurrently with an agent of any one of the preceding Embodiments.

461. The method of Embodiment 456 or 457, wherein a second therapeutic agent or therapy is administered subsequently to an agent of any one of the preceding Embodiments.

462. The method of Embodiment 456 or 457, wherein a second therapeutic agent or therapy is administered about or no more than about 1, 2, 3, 4, 5, 6, or 7 days, or 1, 2, 3, or weeks, or 1, 2, 3, 4, 5, or 6 months, subsequently to an agent of any one of the preceding Embodiments.

463. The method of any one of the preceding Embodiments, wherein a subject is exposed to a second therapeutic agent or therapy and an agent of any one of the preceding Embodiments.

464. The method of any one of the preceding Embodiments, wherein a subject is exposed to a therapeutic effect of a second therapeutic agent or therapy and a therapeutic effect of an agent of any one of the preceding Embodiments.

465. The method of any one of the preceding Embodiments, wherein a second therapeutic agent is or comprises a chemotherapy agent.

466. The method of any one of the preceding Embodiments, wherein a second therapeutic agent is or comprises a hormone therapy agent.

467. The method of any one of the preceding Embodiments, wherein a second therapeutic agent is or comprises an immunotherapy agent.

468. The method of any one of the preceding Embodiments, wherein a second therapeutic agent is or comprises a checkpoint inhibitor.

469. The method of any one of the preceding Embodiments, wherein a second therapeutic agent is or comprises an antibody.

470. The method of any one of the preceding Embodiments, wherein a second therapeutic agent is or comprises a CTLA-4, PD-1 or PD-L1 inhibitor.

471. The method of any one of the preceding Embodiments, wherein a second therapeutic agent is or comprises a cell.

472. The method of any one of the preceding Embodiments, wherein the second therapeutic agent reduces one or more side effects of an agent or composition of any one of the preceding Embodiments.

473. The method of any one of the preceding Embodiments, wherein the agent or composition reduces one or more side effects of a second therapeutic agent.

474. The method of any one of the preceding Embodiments, wherein a second therapy is or comprises surgery.

475. The method of any one of the preceding Embodiments, wherein a second therapy is or comprises chemotherapy.

476. The method of any one of the preceding Embodiments, wherein a second therapy is or comprises radiotherapy.

477. The method of any one of the preceding Embodiments, wherein a second therapy is or comprises hormone therapy.

478. The method of any one of the preceding Embodiments, wherein a second therapy is or comprises stem cell or bone marrow transplant.

479. The method of any one of the preceding Embodiments, wherein a second therapy is or comprises immunotherapy.

480. The method of any one of the preceding Embodiments, wherein a second therapy is or comprises T-cell therapy.

481. The method of any one of the preceding Embodiments, wherein a second therapy is or comprises CAR T-cell therapy.

482. The method of any one of the preceding Embodiments, wherein a second therapy is or comprises administering to the subject a population of immune cells.

483. The method of any one of the preceding Embodiments, wherein the agent or composition reduces one or more side effects of a second therapy.

484. The method of any one of the preceding Embodiments, wherein unit dose of a second therapy or therapeutic agent is reduced compared to when it is administered alone.

485. The method of any one of the preceding Embodiments, wherein total dose of a second therapy or therapeutic agent is reduced compared to when it is administered alone.

486. The method of any one of the preceding Embodiments, wherein unit dose of an agent or composition of any one of the preceding Embodiments is reduced compared to when it is administered alone.

487. The method of any one of the preceding Embodiments, wherein total dose of an agent or composition of any one of the preceding Embodiments is reduced compared to when it is administered alone. 488. The method of any one of the preceding Embodiments, wherein the combination therapy provides higher efficacy than when an agent or composition is administered or delivered alone.

489. The method of any one of the preceding Embodiments, wherein the combination therapy provides higher efficacy than when a second therapeutic agent or therapy is administered or delivered alone. 490. The method of any one of the preceding Embodiments, comprising assessing level of a transcript of a nucleic acid and/or a product thereof.

491. The method of any one of the preceding Embodiments, comprising collecting a sample from a subject, and assessing level of a transcript of a nucleic acid and/or a product thereof in the sample.

492. The method of Embodiment 491, wherein a sample is or comprises a cell, tissue or organ.

493. The method of any one of Embodiments 491-492, wherein a sample is or comprises cancer cells.

494. The method of any one of Embodiments 491-493, wherein a sample is or comprises hepatocellular cancer cells.

495. The method of any one of Embodiments 491-494, wherein a sample comprises cells from a tumor.

496. The method of any one of Embodiments 491-495, wherein a sample comprises tissues from a tumor.

497. The method of any one of Embodiments 491-496, wherein a sample is or comprises a tumor.

498. The method of any one of Embodiments 491-497, wherein a sample is from a tumor.

499. The method of any one of Embodiments 491-498, wherein a sample is from a biopsy.

500. The method of any one of Embodiments 491-499, wherein a sample is collected after one or more administrations or deliveries.

501. The method of any one of Embodiments 491-500, wherein an assessment is conducted after one or more administrations or deliveries.

502. The method of any one of Embodiments 491-501, wherein a nucleic acid is or comprises a gene.

503. The method of any one of Embodiments 491-502, wherein a nucleic acid is selected from gene set BCAT_GDS748_UP, BCAT.100_UP.V1_UP, HALLMARK_WNT_BETA-CATENIN_SIGNALING, RASHI-RESPONSE_TO_IONIZING-RADIATION_1, REACTOME-RRNA_PROCESSING, HALLMARK_MYC_TARGETS_V1, HALLMARK_MYC_TARGETS_V2, HALLMARK_OXIDATIVE_PHOSPHORYLATION, HALLMARK_E2F_TARGETS, or HALLMARK_TNFA_SIGNALING_VIA_NFKB.

504. The method of any one of Embodiments 491-503, wherein a nucleic acid is CCND2.

505. The method of any one of Embodiments 491-504, wherein a nucleic acid is WNT5B.

506. The method of any one of Embodiments 491-505, wherein a nucleic acid is AXIN2.

507. The method of any one of Embodiments 491-506, wherein a nucleic acid is NKD1.

508. The method of any one of Embodiments 491-507, wherein a nucleic acid is WNT6.

509. The method of any one of Embodiments 491-508, wherein a nucleic acid is DKK1.

510. The method of any one of Embodiments 491-503, wherein the nucleic acid is DKK4.

511. The method of any one of Embodiments 491-510, wherein administration or delivery continues for one or more times after the assessment.

512. The method of any one of Embodiments 491-511, comprising evaluating an assessment and continue the administration or delivery.

513. The method of any one of Embodiments 491-512, wherein administration or deliver is adjusted after an assessment.

514. The method of any one of Embodiments 491-513, comprising evaluating an assessment and adjusting the administration or delivery.

515. The method of any one of Embodiments 491-502, wherein administration or deliver is discontinued after an assessment.

516. The method of any one of Embodiments 491-501 and 515, comprising evaluating an assessment and discontinuing the administration or delivery.

517. An agent, compound, composition or method of any one of the preceding Embodiments, wherein a tissue, organ, subject or system comprises a hepatocellular cancer cell that comprises another mutation.

518. An agent, compound, composition or method of any one of the preceding Embodiments, wherein a tissue, organ, subject or system comprises a hepatocellular cancer cell that comprises a MSH6 mutation.

519. An agent, compound, composition or method of any one of the preceding Embodiments, wherein a tissue, organ, subject or system comprises a hepatocellular cancer cell that is MSI-H.

520. An agent, compound, or composition of any one of the preceding Embodiments, prepared and/or characterized by a method of any one of the preceding Embodiments.

### EXEMPLIFICATION

Those skilled in the art appreciate that various technologies are available for manufacturing and assessing provided agents including various peptides such as stapled peptides in accordance with the present disclosure, for example, many technologies for preparing small molecules and peptides can be utilized to prepare provided agents, and various assays are available for assessing properties and/or activities of provided agents. Described below are certain such useful technologies. As demonstrated herein, in some embodiments, it is confirmed that provided technologies can exhibit nanomolar cell-based activity in protein-protein interaction (PPI), transcriptional regulation, proliferation assays, etc. In some embodiments, it is confirmed that provided technologies possess favorable pharmacokinetic properties. In some embodiments, in vivo dosing of provided technologies confirms on-target pharmacodynamic modulation of β-catenin activity and strong anti-tumor activity in multiple human xenograft models, which confirm that provided technologies are useful for treating various conditions, disorders or diseases as described herein.

### Example 1. Preparation of Compounds

Among other things, peptides can be prepared using various peptide synthesis technologies in accordance with the present disclosure. In many embodiments, peptides were prepared using Fmoc-based synthesis, often on suitable solid phase. For various stapled peptides, amino acid residues were stapled through suitable chemistry, e.g., olefin metathesis for amino acids that comprise olefin groups. Those skilled in the art appreciates that other suitable technologies may also be utilized for stapling in accordance with the present disclosure, e.g., those described in the priority applications, WO/2019/051327, WO/2020/041270, WO 2022/261257 etc., the peptide staples and technologies for preparing peptides are incorporated herein by reference.

### Example 2. Provided Technologies can Provide Improved Properties and/or Activities

Various technologies can be utilized to assess technologies of the present disclosure. For example, in some embodiments, solubility, LogD, interactions with targets, etc. of various agents such as stapled peptides were assessed using technologies described in WO 2022/261257 (e.g., those in its Examples). In some embodiments, solubility, shake flask LogD, and binding to beta-catenin were assessed. In some embodiments, an assay for assessing interactions, e.g., with beta-catenin, was fluorescence polarization as described in WO 2022/261257. In some embodiments, an assay was Nano-BRET target engagement assay that assessed beta-catenin/TCF4 engagement as described in WO 2022/261257. In some embodiments, an assay was a reporter assay as reported in Example 4 of WO 2022/261257. In some embodiments, an assay was a COLO320DM proliferation assay as reported in Example 4 of WO 2022/261257. Certain results are presented in Tables below as examples.

TABLE E1
Certain data of various compositions as examples.

| 1 | 2 | 3 | 4 | 5 | 6 | 7 | 8 | 9 |
|---|---|---|---|---|---|---|---|---|
| I-1 | A | D | | | 1899.8 | 1901.4 | 1899.3 | |
| I-2 | A | | | | 1899.8 | 1901.3 | 1899.4 | |
| I-3 | A | C | | | 1899.8 | 1901.3 | 1899.4 | |
| I-4 | A | D | | | 1899.8 | 951.3 | 1899.5 | |
| I-5 | A | D | | | 1899.8 | 1901.3 | 1899.4 | |
| I-6 | A | D | | | 1899.8 | 951.2 | 1899.3 | |
| I-7 | A | D | | | 1956.8 | 1958.4 | 1956.5 | |
| I-8 | A | D | | | 1956.8 | 1958.4 | 1956.5 | |
| I-9 | A | C | | | 1975.9 | 1977.4 | 1975.4 | |
| I-10 | A | D | | | 1975.9 | 1977.3 | 1975.4 | |
| I-11 | A | B | | | 1975.9 | 1977.4 | 1975.5 | |
| I-12 | A | C | | | 1975.9 | 1977.3 | 1975.5 | |
| I-13 | A | C | | | 1975.9 | 1977.3 | 1975.4 | |
| I-14 | A | C | | | 1975.9 | 1977.4 | 1975.5 | |
| I-15 | A | B | | | 1975.9 | 989.2 | 1975.4 | |
| I-16 | A | C | | | 1975.9 | 989.3 | 1875.5 | |
| I-17 | A | D | | | 1923.8 | 1925.5 | 1923.3 | |
| I-18 | A | | | | 1923.8 | 1925.3 | 1923.3 | |
| I-19 | A | C | | | 1999.9 | 2001.4 | 1999.3 | |
| I-20 | A | | | | 1999.9 | 2001.3 | 1999.3 | |
| I-21 | A | B | | | 2061.9 | 2063.3 | 2061.3 | |
| I-22 | A | | | | 2061.9 | 2063.4 | 2061.4 | |
| I-23 | A | C | | | 1923.8 | 1925.2 | 1923.2 | |
| I-24 | A | C | ++ | | 1999.9 | 2001.3 | 1999.4 | |
| I-25 | A | C | | | 2061.9 | 1032.2 | 2061.3 | |
| I-26 | D | | | | 1980.9 | 1982.3 | 1980.3 | |
| I-27 | C | | | | 2056.9 | 2058.3 | 2056.4 | |
| I-28 | D | | | | 2118.9 | 2120.2 | 2118.4 | |
| I-29 | A | D | | | 1980.9 | 1982.3 | 1980.3 | |
| I-30 | A | D | | | 2056.9 | 2058.5 | 2056.5 | |
| I-31 | A | D | | | 2118.9 | 2120.3 | 2118.4 | |
| I-32 | A | A | | | 1989.9 | 1992.0 | 1990.1 | |
| I-33 | A | C | | | 1989.9 | 996.6 | 1990.1 | |
| I-34 | A | C | | | 1975.9 | 1977.9 | 1976.0 | |
| I-35 | D | | | | 1975.9 | 1977.9 | 1976.1 | |
| I-36 | C | | | | 1975.9 | 1977.9 | 1975.9 | |
| I-37 | A | C | | | 1975.9 | 1977.9 | 1976.2 | |
| I-38 | A | C | | | 1975.9 | 989.6 | 1976.3 | |
| I-39 | A | D | | | 1961.8 | 1964.0 | 1961.9 | |
| I-40 | E | | | | 1961.8 | 1963.9 | 1961.7 | |
| I-41 | C | | | | 1961.8 | 1963.9 | 1961.9 | |
| I-42 | A | B | | | 2060.9 | 2063.1 | 2061.1 | |
| I-43 | A | B | | | 2060.9 | 1032.2 | 2060.9 | |
| I-44 | D | | | | 2046.9 | 2049.0 | 2046.9 | |
| I-45 | A | D | | | 2179.0 | 2181.3 | 2179.4 | |
| I-46 | A | C | | | 2131.0 | 2133.4 | 2131.5 | |
| I-47 | A | D | | | 2145.0 | 2147.3 | 2145.4 | |
| I-48 | A | D | | | 2255.0 | 2257.6 | 2255.1 | |
| I-49 | B | C | | | 2255.0 | 2257.4 | 2255.4 | |
| I-50 | B | D | | | 2207.0 | 1105.4 | 2208.2 | |
| I-51 | A | C | | | 2159.0 | 2161.7 | 2159.7 | |
| I-52 | A | D | | | 2173.0 | 2175.5 | 2173.7 | |
| I-53 | B | D | | | 2283.0 | 2285.6 | 2283.9 | |
| I-54 | B | | | | 2283.0 | 2285.7 | 2283.6 | |
| I-55 | A | B | | | 2051.9 | 2053.9 | 2052.3 | |
| I-56 | A | C | | | 2037.9 | 2039.9 | 2037.8 | |
| I-57 | A | B | | | 2122.9 | 2125.0 | 2123.1 | |
| I-58 | B | D | | | 2108.9 | 2111.1 | 2108.8 | |
| I-59 | B | D | | | 2108.9 | 2111.1 | 2109.1 | |
| I-60 | A | C | | | 2180.0 | 2182.1 | 2180.1 | |
| I-61 | A | D | | | 2165.9 | 2168.2 | 2165.9 | |
| I-62 | A | C | | | 2077.9 | 2080.1 | 2078.2 | |
| I-63 | A | C | | | 2077.9 | 2080.0 | 2078.2 | |
| I-64 | A | B | ++ | ++ | 2073.9 | 2076.1 | 2074.0 | |
| I-65 | A | B | | | 2047.9 | 2050.0 | 2048.0 | |
| I-66 | A | A | | | 2074.9 | 1038.8 | 1036.8 | |
| I-67 | A | A | + | + | 2074.9 | 2076.6 | 2074.7 | |
| I-68 | A | B | | | 2137.0 | 1069.7 | 1067.8 | |
| I-69 | A | A | + | + | 2137.0 | 2138.7 | 2136.8 | |
| I-70 | A | A | + | + | 2103.0 | 2104.6 | 2102.8 | |
| I-71 | A | B | | | 2165.0 | 2166.7 | 2164.8 | |
| I-72 | A | B | | | 2090.9 | 2092.5 | 2090.6 | |
| I-73 | A | B | | | 2152.9 | 2154.6 | 2152.7 | |
| I-74 | A | D | | | 2101.9 | 2103.5 | 2101.6 | |
| I-75 | A | D | | | 2163.9 | 2165.5 | 2163.6 | |
| I-76 | A | C | | | 2064.9 | 2066.6 | 2064.6 | A |
| I-77 | A | D | | | 2127.0 | 2128.8 | 2126.8 | A |
| I-78 | A | D | | | 2105.9 | 2107.6 | 2105.8 | A |
| I-79 | B | | | | 2168.0 | 2169.8 | 2167.9 | A |
| I-80 | A | C | | | 2059.9 | 2061.6 | 2059.8 | |
| I-81 | B | C | | | 2046.9 | 2048.5 | 2046.6 | |
| I-82 | A | C | | | 2059.9 | 2061.5 | 2059.6 | |
| I-83 | C | D | | | 2046.9 | 2048.5 | 2046.6 | |
| I-84 | A | C | | | 2075.9 | 2077.6 | 2075.7 | |
| I-85 | B | D | | | 2062.9 | 2064.5 | 2062.6 | |
| I-86 | B | D | | | 2075.9 | 2077.7 | 2075.8 | |
| I-87 | C | | | | 2062.9 | 2064.6 | 2062.7 | |
| I-88 | A | D | | | 2116.9 | 2118.8 | 2116.8 | |
| I-89 | B | D | | | 2103.9 | 2105.7 | 2103.8 | |
| I-90 | B | D | | | 2116.9 | 2118.6 | 2116.7 | |
| I-91 | C | | | | 2103.9 | 2105.7 | 2103.7 | |
| I-92 | A | | | | 2236.0 | 1119.3 | 1117.3 | |
| I-93 | C | | | | 2248.0 | 1125.6 | 1123.4 | |
| I-94 | C | | | | 2248.0 | 1125.6 | 1123.5 | |
| I-95 | B | | | | 2276.1 | 2277.5 | 2275.6 | |
| I-96 | C | | | | 2276.1 | 1140.5 | 1138.6 | |
| I-97 | A | B | | | 2149.0 | 2150.5 | 2148.6 | |
| I-98 | A | C | | | 2149.0 | 1075.8 | 1073.9 | |
| I-99 | A | D | + | | 2179.0 | 1090.8 | 2178.4 | |
| I-100 | A | | | | 2179.0 | 1090.8 | 1088.9 | |
| I-101 | A | B | | | 2205.0 | 1103.8 | 2204.4 | |
| I-102 | A | C | | | 2205.0 | 1103.8 | 2204.4 | |
| I-103 | A | C | | | 2231.0 | 1116.8 | 2230.5 | |
| I-104 | A | B | | | 2165.0 | 1083.8 | 2164.4 | |
| I-105 | A | | | | 2165.0 | 1083.8 | 1081.9 | |
| I-106 | A | B | + | | 2191.0 | 1096.8 | 2190.4 | |
| I-107 | A | | | | 2191.0 | 1097.0 | 1095.0 | |
| I-108 | A | D | | | 2221.0 | 1111.8 | 2220.5 | |
| I-109 | B | D | ++ | | 2221.0 | 1111.8 | 2220.5 | |
| I-110 | B | C | | | 2247.1 | 1124.8 | 2246.5 | |
| I-111 | B | C | | | 2247.1 | 1224.8 | 2246.5 | |
| I-112 | A | C | | | 2157.9 | 1080.3 | 1078.3 | |
| I-113 | A | D | | | 2157.0 | 1079.1 | 2156.3 | |
| I-114 | B | | | | 2157.0 | 1079.8 | 2156.3 | |
| I-115 | E | | | | 2144.0 | 1073.3 | 2143.3 | |
| I-116 | E | | | | 2144.0 | 1073.3 | 2143.3 | |
| I-117 | C | | | | 2157.0 | 2158.2 | 2156.3 | |
| I-118 | E | | | | 2144.0 | 1073.3 | 2143.3 | |
| I-119 | E | | | | 2144.0 | 1073.3 | 2143.3 | |
| I-120 | A | B | | | 2172.0 | 2173.2 | 2171.3 | |
| I-121 | A | B | ++ | | 2172.0 | 2173.2 | 2171.3 | |
| I-122 | A | D | | | 2200.0 | 2201.2 | 2199.3 | |
| I-123 | B | D | | | 2234.0 | 1118.3 | 2233.4 | |
| I-124 | A | D | | | 2186.0 | 1094.3 | 1092.3 | |
| I-125 | A | D | ++ | | 2186.0 | 1094.3 | 1092.3 | |
| I-126 | A | C | ++ | | 2172.0 | 1087.2 | 2171.3 | |
| I-127 | A | C | ++ | | 2172.0 | 1087.2 | 2171.3 | |
| I-128 | B | C | ++ | | 2200.0 | 2201.2 | 2199.4 | |
| I-129 | A | C | ++ | | 2186.0 | 2187.2 | 2185.3 | |
| I-130 | B | D | | | 2302.1 | 1152.3 | − | |
| I-131 | C | D | | | 2302.1 | 2303.4 | − | |
| I-132 | B | D | | | 2302.1 | 2303.5 | − | |
| I-133 | D | D | | | 2306.1 | 2307.5 | − | A |
| I-134 | C | D | | | 2306.1 | 2303.3 | − | A |
| I-135 | E | D | | | 2306.1 | 2307.6 | − | A |
| I-136 | A | B | | | 2252.0 | 1127.6 | 1125.6 | |
| I-137 | A | B | | | 2294.1 | 1148.6 | 1146.6 | |
| I-138 | A | B | + | + | 2266.0 | 1134.6 | 1132.6 | |
| I-139 | A | B | + | | 2264.0 | 1133.6 | 1131.6 | |
| I-140 | A | A | + | | 2278.0 | 1140.6 | 1138.6 | |
| I-141 | A | B | + | | 2292.0 | 1147.6 | 1145.5 | |
| I-142 | C | C | +++ | | 2251.0 | 1127.0 | 1125.0 | |
| I-143 | C | D | | | 2293.1 | 1148.1 | 1146.1 | |
| I-144 | A | C | ++ | | 2265.0 | 1134.1 | 1132.3 | |
| I-145 | E | | | | 2238.0 | 1120.5 | 1118.6 | |
| I-146 | E | | | | 2280.1 | 2282.1 | 2280.2 | |
| I-147 | D | | | | 2252.1 | 1127.6 | 1125.6 | |
| I-148 | C | | | | 1976.0 | 989.2 | 987.0 | |
| I-149 | C | D | | | 2045.0 | 2046.7 | 2044.6 | |
| I-150 | E | | | | 2018.0 | 2019.4 | 2017.4 | |
| I-151 | B | D | | | 1981.9 | 993.3. | 991.2. | |
| I-152 | D | | | | 2051.0 | 2011.4 | 2009.4 | |
| I-153 | E | | | | 1980.0 | 991.3. | 989.2. | A |
| I-154 | E | | | | 2049.1 | 2050.6 | 2048.4 | A |
| I-155 | E | | | | 1953.0 | 977.7 | 975.5 | A |
| I-156 | E | | | | 2022.1 | 2023.6 | 2021.5 | A |
| I-157 | E | | | | 1985.9 | 994.2. | 1985.3 | A |
| I-158 | E | | | | 2055.0 | 2056.6 | 2054.5 | A |
| I-159 | A | B | | | 2073.9 | 1038.3 | 1036.2 | |
| I-160 | A | C | | | 2046.9 | 1024.8 | 1022.9 | |
| I-161 | C | | | | 2060.9 | 1031.8 | 1029.6 | |
| I-162 | C | D | | | 2060.9 | 2062.0 | 2060.1 | |
| I-163 | A | C | +++ | | 2060.9 | 1031.7 | 2060.1 | |
| I-164 | B | D | | | 2088.0 | 1045.2 | 2087.2 | |
| I-165 | A | C | | | 2073.9 | 1038.2 | 2073.2 | |
| I-166 | B | C | | | 2046.9 | 1024.7 | 2046.1 | |
| I-167 | E | | | | 2060.9 | 1031.7 | 2060.2 | |
| I-168 | C | D | | | 2060.9 | 1031.7 | 2060.2 | |
| I-169 | B | D | | | 2060.9 | 1031.7 | 2060.2 | |
| I-170 | B | D | | | 2088.0 | 1045.2 | 2087.2 | |
| I-171 | B | D | | | 2078.0 | 1040.2 | 2077.2 | A |
| I-172 | D | | | | 2051.0 | 1026.7 | 2050.3 | A |
| I-173 | E | | | | 2065.0 | 1033.7 | 2064.2 | A |
| I-174 | E | | | | 2065.0 | 1033.7 | 2064.2 | A |
| I-175 | E | | | | 2065.0 | 1033.7 | 2064.2 | A |
| I-176 | E | | | | 2092.0 | 1047.2 | 2091.2 | A |
| I-177 | C | D | | | 2078.0 | 1040.2 | 2077.2 | A |
| I-178 | E | | | | 2051.0 | 1026.7 | 2050.2 | A |
| I-179 | E | | | | 2065.0 | 1033.7 | 2064.2 | A |
| I-180 | E | | | | 2065.0 | 1033.7 | 2064.2 | A |
| I-181 | E | | | | 2065.0 | 1033.7 | 2064.2 | A |
| I-182 | E | | | | 2092.0 | 1047.2 | 1887.9 | A |
| I-183 | A | C | ++ | | 2074.9 | 1038.7 | 1036.8 | |
| I-184 | A | | | | 2137.0 | 1069.7 | 1067.7 | |
| I-185 | A | | | | 2074.9 | 1038.7 | 1036.8 | |
| I-186 | A | | | | 2137.0 | 1069.7 | 1067.6 | |
| I-187 | A | C | +++ | | 2117.0 | 1059.7 | 1057.6 | |
| I-188 | A | C | | | 2179.0 | 1090.7 | 1088.8 | |
| I-189 | A | C | | | 2179.0 | 1090.7 | 1088.7 | |
| I-190 | A | | | | 2103.0 | 1052.7 | 1050.8 | |
| I-191 | B | C | | | 2165.0 | 1083.7 | 1081.7 | |
| I-192 | A | | | | 2165.0 | 1083.7 | 1081.7 | |
| I-193 | A | C | | | 2152.9 | 2175.8 | 2152.0 | |
| I-194 | A | A | | | 2152.9 | 2175.8 | 2152.0 | |
| I-195 | A | | | | 2184.0 | 2184.9 | 2183.1 | |
| I-196 | B | C | | | 2137.0 | 1069.6 | 2136.1 | |
| I-197 | A | B | | | 2137.0 | 2159.8 | 2136.0 | |
| I-198 | A | C | | | 2074.9 | 2097.8 | 2073.9 | |
| I-199 | A | C | | | 2060.9 | 2083.8 | 2060.0 | |
| I-200 | B | D | | | 2046.9 | 2069.8 | 2046.1 | |
| I-201 | B | C | | | 2074.0 | 2096.8 | 2073.0 | |
| I-202 | A | D | | | 2083.0 | 2084.1 | 2082.0 | |
| I-203 | E | D | | | 2109.0 | 2131.9 | 2108.1 | |
| I-204 | A | C | | | 2204.9 | 1103.6 | 1101.7 | |
| I-205 | A | C | | | 2204.9 | 2227.8 | 2204.0 | |
| I-206 | A | | | | 2204.9 | 2227.8 | 2204.0 | |
| I-207 | A | B | | | 2204.9 | 2227.8 | 2204.0 | |
| I-208 | A | C | | | 2154.9 | 2177.8 | 2154.0 | |
| I-209 | E | D | | | 2045.0 | 2067.8 | 2044.0 | |
| I-210 | B | C | | | 2032.9 | 2055.8 | 2032.0 | |
| I-211 | C | D | | | 2132.0 | 2154.9 | 2131.1 | |
| I-212 | A | C | | | 2059.9 | 2082.8 | 2059.0 | |
| I-213 | B | C | | | 2016.9 | 2039.8 | 2015.9 | |
| I-214 | B | D | | | 2102.0 | 2103.2 | 2101.1 | |
| I-215 | A | | | | 2074.9 | 1038.7 | 1036.7 | |
| I-216 | B | C | | | 2046.9 | 1024.7 | 1022.6 | |
| I-217 | E | D | | | 2123.0 | 1062.7 | 1060.8 | |
| I-218 | B | C | | | 2136.0 | 1069.2 | 1067.2 | |
| I-219 | C | | | | 2059.0 | 1030.7 | 1028.7 | |
| I-220 | C | D | | | 2093.0 | 1047.7 | 1045.5 | |
| I-221 | C | D | | | 2074.0 | 2075.2 | 2073.2 | |
| I-222 | E | D | | | 2059.0 | 1030.7 | 1028.6 | |
| I-223 | A | B | | | 2211.0 | 2212.2 | 2210.2 | |
| I-224 | A | D | | | 2324.1 | 2325.3 | 2323.2 | |
| I-225 | A | | | | 2308.1 | 2309.3 | 2307.2 | |
| I-226 | A | B | | | 2165.0 | 1083.7 | 1081.8 | |
| I-227 | A | B | | | 2252.0 | 2253.1 | 2251.2 | |
| I-228 | A | C | | | 2278.1 | 2279.3 | 2277.3 | |
| I-229 | A | D | | | 2365.1 | 2366.4 | 2364.3 | |
| I-230 | A | | | | 2224.0 | 1113.2 | 1111.2 | |
| I-231 | A | D | | | 2250.0 | 2251.1 | 2049.2 | |
| I-232 | A | D | | | 2337.1 | 2338.1 | 2336.2 | |
| I-233 | A | D | | | 2010.9 | 1006.6 | 2009.9 | A |
| I-234 | A | D | | | 2024.9 | 1013.6 | 2023.9 | A |
| I-235 | A | D | | | 2038.9 | 1020.6 | 2038.1 | A |
| I-236 | A | D | | | 2010.9 | 1006.6 | 2009.9 | A |
| I-237 | A | D | | +++ | 1996.9 | 999.6. | 1995.8 | A |
| I-238 | A | D | | | 2010.9 | 1006.6 | 2009.9 | A |
| I-239 | C | D | | | 2045.9 | 2046.8 | 2044.8 | A |
| I-240 | B | D | | | 2059.9 | 1031.1 | 2058.9 | A |
| I-241 | C | D | | | 2073.9 | 1038.1 | 2073.2 | A |
| I-242 | C | D | | | 2045.9 | 1024.1 | 2044.8 | A |
| I-243 | B | D | | | 2031.9 | 1017.1 | 2030.8 | A |
| I-244 | B | D | | | 2045.9 | 1024.1 | 2045.2 | A |
| I-245 | A | B | | + | 2060.9 | 1031.6 | 2059.9 | |
| I-246 | A | B | | | 2060.9 | 1031.6 | 2060.2 | |
| I-247 | A | B | | | 2060.9 | 1031.6 | 2060.2 | |
| I-248 | A | B | | | 2046.9 | 1024.6 | 2045.9 | |
| I-249 | A | C | | | 2094.9 | 1048.6 | 2093.9 | |
| I-250 | A | C | | | 2100.9 | 1051.6 | 2100.2 | |
| I-251 | A | B | | | 2086.9 | 1044.6 | 2085.9 | |
| I-252 | A | C | | | 2058.9 | 1030.6 | 2057.9 | |
| I-253 | A | A | | + | 2086.9 | 1044.6 | 2086.2 | |
| I-254 | A | C | | | 2184.8 | 1093.5 | 2183.9 | |
| I-255 | A | C | | | 2074.9 | 1038.6 | 2073.9 | |
| I-256 | A | C | | | 2086.9 | 1044.6 | 2086.2 | |
| I-257 | A | C | | | 2149.0 | 1075.6 | 2147.9 | |
| I-258 | A | C | | | 2086.9 | 1044.6 | 2085.9 | |
| I-259 | B | C | | | 2149.0 | 1075.6 | 2147.9 | |
| I-260 | A | | | | 2100.9 | 1082.5 | 2161.9 | |
| I-261 | A | | | | 2163.0 | 1051.6 | 2100.2 | |
| I-262 | A | B | + | + | 2114.9 | 1058.6 | 2114.2 | |
| I-263 | A | C | | | 2114.9 | 1058.6 | 2113.9 | |
| I-264 | A | | | | 2100.9 | 1051.6 | 2099.9 | |
| I-265 | A | C | | | 2100.9 | 1051.6 | 2099.9 | |
| I-266 | C | D | | | 1992.9 | 1996.6 | 1994.8 | |
| I-267 | C | | | | 1992.9 | 1993.9 | 1992.0 | |
| I-268 | B | D | | | 2006.9 | 1005.7 | 2008.3 | |
| I-269 | B | D | | | 2006.9 | 1004.6 | 1002.6 | |
| I-270 | A | C | | | 2021.0 | 1011.6 | 1009.7 | |
| I-271 | A | C | | | 2035.0 | 1019.8 | 2036.6 | |
| I-272 | A | B | | | 2035.0 | 1018.6 | 1016.8 | |
| I-273 | B | D | | | 2035.0 | 1019.8 | 2036.8 | |
| I-274 | B | D | | | 2035.0 | 1018.6 | 1016.6 | |
| I-275 | A | C | | | 2049.0 | 1026.7 | 2050.4 | |
| I-276 | A | C | | | 2049.0 | 1025.6 | 1023.7 | |
| I-277 | B | D | | | 2049.0 | 1026.8 | 2050.4 | |
| I-278 | B | D | | | 2049.0 | 1025.7 | 1023.8 | |
| I-279 | A | C | | | 2069.0 | 1036.8 | 2070.8 | |
| I-280 | A | B | | | 2069.0 | 2070.1 | 2068.2 | |
| I-281 | A | C | | | 2033.0 | 1018.7 | 2034.4 | |
| I-282 | A | C | | | 2033.0 | 1017.6 | 1015.7 | |
| I-283 | A | C | | | 2047.0 | 1024.6 | 1022.7 | |
| I-284 | A | C | | | 2061.0 | 1031.6 | 1029.4 | |
| I-285 | A | C | | | 2075.0 | 1038.6 | 1036.7 | |
| I-286 | A | | | | 2151.0 | 2152.1 | 2150.2 | |
| I-287 | B | D | | | 2151.0 | 1076.7 | 2150.0 | |
| I-288 | A | B | | | 2151.0 | 2151.9 | 2150.0 | |
| I-289 | A | C | | | 2151.0 | 1076.6 | 1074.6 | |
| I-290 | B | | | | 2151.0 | 1076.7 | 2150.3 | |
| I-291 | E | D | ++ | | 2165.0 | 1083.7 | 2164.0 | |
| I-292 | A | C | | | 2058.9 | 2060.0 | 2058.0 | |
| I-293 | A | B | | | 2099.0 | 2099.9 | 2098.1 | |
| I-294 | A | A | | + | 2083.0 | 2083.9 | 2082.1 | |
| I-295 | A | A | | | 2146.9 | 1075.0 | 1073.1 | |
| I-296 | A | B | | | 2102.9 | 1052.8 | 1050.9 | |
| I-297 | A | B | | | 2094.0 | 2094.9 | 2093.1 | |
| I-298 | A | A | | | 2113.9 | 2114.9 | 2113.0 | |
| I-299 | A | B | | ++ | 2070.0 | 2071.2 | 2069.1 | |
| I-300 | A | C | | +++ | 2070.0 | 2071.2 | 2069.0 | |
| I-301 | A | D | | +++ | 2070.0 | 2071.1 | 2069.1 | |
| I-302 | A | D | | | 2059.0 | 2060.0 | 2058.0 | |
| I-303 | A | D | | | 2843.3 | 1422.9 | 1420.8 | |
| I-304 | A | C | | + | 2018.9 | 1010.6 | 2017.9 | |
| I-305 | A | C | | | 2110.9 | 1056.6 | 2110.0 | |
| I-306 | A | D | | | 2133.9 | 1068.1 | 2133.0 | |
| I-307 | A | D | | | 2034.9 | 1018.6 | 2033.9 | |
| I-308 | A | | | | 2032.9 | 2034.1 | 2032.1 | |
| I-309 | A | | | | 2080.9 | 2082.1 | 2080.1 | |
| I-310 | A | B | | | 2170.9 | 1086.6 | 2170.1 | |
| I-311 | A | B | | | 2117.0 | 1059.6 | 2116.0 | |
| I-312 | A | B | | | 2072.9 | 1037.6 | 2072.0 | |
| I-313 | A | D | | +++ | 2131.9 | 2133.1 | 2131.0 | |
| I-314 | A | C | | | 2165.9 | 2167.2 | 2165.3 | |
| I-315 | A | C | | | 2121.0 | 2122.1 | 2120.3 | |
| I-316 | B | D | | | 2094.0 | 2095.2 | 2093.3 | |
| I-317 | E | | | | 2108.0 | 2109.1 | 2107.3 | |
| I-318 | A | C | | | 2121.0 | 2122.1 | 2120.3 | |
| I-319 | A | D | | | 2234.0 | 2235.3 | 2233.4 | |
| I-320 | B | D | | | 2207.0 | 2208.3 | 2206.4 | |
| I-321 | E | | | | 2221.0 | 2222.2 | 2220.3 | |
| I-322 | A | C | | | 2234.0 | 2235.3 | 2233.4 | |
| I-323 | A | B | | | 2187.0 | 2188.2 | 2186.1 | |
| I-324 | A | D | | | 2160.0 | 2161.1 | 2159.3 | |
| I-325 | C | | | | 2174.0 | 2175.1 | 2173.2 | |
| I-326 | A | C | | | 2187.0 | 2188.2 | 2186.3 | |
| I-327 | A | | | | 2148.9 | 2150.6 | 2148.7 | |
| I-328 | A | B | | | 2086.9 | 2088.6 | 2086.8 | |
| I-329 | A | A | | | 2080.9 | 2082.6 | 2080.8 | |
| I-330 | A | C | | | 2109.0 | 1055.6 | 1053.6 | |
| I-331 | A | | | | 2158.9 | 2160.8 | 2158.9 | |
| I-332 | A | C | | | 2096.9 | 2098.7 | 2096.9 | |
| I-333 | A | | | | 2090.9 | 1047.0 | 1044.9 | |
| I-334 | A | B | | | 2119.0 | 1060.6 | 1058.6 | |
| I-335 | A | A | + | + | 2084.9 | 2085.9 | 2084.0 | |
| I-336 | A | B | ++ | | 2084.9 | 1043.6 | 2084.1 | |
| I-337 | A | B | + | | 2070.9 | 2071.9 | 2070.0 | |
| I-338 | A | C | + | | 2070.9 | 1036.6 | 2070.0 | |
| I-339 | C | D | | | 2249.8 | 2250.7 | 2248.9 | |
| I-340 | C | | | | 2397.8 | 2398.7 | 2396.8 | |
| I-341 | C | D | | | 2397.8 | 2398.8 | 2396.9 | |
| I-342 | A | B | + | | 2204.0 | 1103.1 | 1101.2 | |
| I-343 | A | B | | | 2275.0 | 1138.6 | 1136.5 | |
| I-344 | A | D | | | 2113.0 | 2113.9 | 2112.1 | |
| I-345 | A | C | | | 2118.9 | 2119.9 | 2118.1 | |
| I-346 | B | | | | 2156.9 | 2157.9 | 2156.0 | |
| I-347 | B | D | | | 2357.0 | 1179.6 | 1177.6 | |
| I-348 | A | | | | 2204.9 | 2206.0 | 2204.1 | |
| I-349 | A | D | | | 2113.9 | 2114.8 | 2113.0 | |
| I-350 | A | D | | | 2119.9 | 2120.9 | 2119.0 | |
| I-351 | B | D | | | 2157.9 | 2158.8 | 2156.9 | |
| I-352 | B | D | | | 2358.0 | 2358.9 | 2357.0 | |
| I-353 | A | | | | 2052.9 | 1027.6 | 1025.6 | |
| I-354 | B | D | | | 2067.0 | 2067.9 | 2066.1 | |
| I-355 | A | C | | | 2130.9 | 1066.6 | 1064.7 | |
| I-356 | A | D | | | 2124.9 | 2125.9 | 2124.1 | |
| I-357 | A | C | | | 2124.9 | 1063.7 | 1061.9 | |
| I-358 | A | B | | | 2090.9 | 2091.9 | 2090.1 | |
| I-359 | A | C | | | 2113.9 | 2114.9 | 2113.0 | |
| I-360 | D | | | | 2040.9 | 2041.9 | 2040.1 | |
| I-361 | E | | | | 2040.9 | 1021.6 | 1019.8 | |
| I-362 | A | | | | 2040.9 | 1021.6 | 1019.5 | |
| I-363 | B | D | | | 2014.9 | 2015.9 | 2014.0 | |
| I-364 | A | A | | | 2080.9 | 2082.0 | 2080.3 | |
| I-365 | A | B | | | 2080.9 | 2081.9 | 2080.0 | |
| I-366 | E | | | | 2067.0 | 1034.6 | 1032.7 | |
| I-367 | B | | | | 2028.9 | 2029.9 | 2028.0 | |
| I-368 | C | | | | 2065.9 | 2067.0 | 2065.1 | |
| I-369 | A | C | | | 2142.9 | 1072.6 | 1070.6 | |
| I-370 | E | | | | 2028.9 | 2029.9 | 2028.0 | |
| I-371 | C | | | | 2064.9 | 2066.1 | 2064.0 | |
| I-372 | E | | | | 2026.9 | 2027.9 | 2026.0 | |
| I-373 | E | | | | 2041.9 | 2042.9 | 2041.0 | |
| I-374 | C | | | | 2055.9 | 2056.9 | 2055.1 | |
| I-375 | A | C | | + | 2205.9 | 1104.2 | 1102.2 | |
| I-376 | C | D | | | 2205.9 | 1104.2 | 1102.1 | |
| I-377 | B | C | | | 2205.9 | 1104.1 | 1102.1 | |
| I-378 | A | B | | | 2188.0 | 1095.1 | 1093.2 | |
| I-379 | A | B | | | 2146.0 | 1074.1 | 1072.3 | |
| I-380 | A | D | | | 2152.9 | 1077.6 | 1075.7 | |
| I-381 | A | B | | | 2160.0 | 1081.1 | 1079.3 | |
| I-382 | A | B | | | 2100.9 | 1051.6 | 1050.0 | |
| I-383 | A | A | | | 2100.9 | 1051.6 | 1049.6 | |
| I-384 | A | | | | 2115.9 | 2117.2 | 2115.1 | |
| I-385 | A | D | | | 2115.9 | 2117.1 | 2115.1 | |
| I-386 | A | A | | +++ | 2136.9 | 1069.6 | 1067.6 | |
| I-387 | A | C | | | 2126.9 | 2128.1 | 2126.1 | |
| I-388 | A | A | | | 2088.9 | 1045.6 | 1043.6 | |
| I-389 | A | B | | | 2076.9 | 1039.6 | 1037.7 | |
| I-390 | A | B | | | 2076.9 | 1039.6 | 1037.7 | |
| I-391 | A | C | | | 2117.9 | 1060.1 | 1058.2 | |
| I-392 | A | C | | | 2117.9 | 1060.1 | 1058.2 | |
| I-393 | A | C | | | 2103.9 | 1053.1 | 1051.2 | |
| I-394 | A | | | | 2103.9 | 2104.7 | 2102.9 | |
| I-395 | E | | | | 2024.9 | 2025.9 | 2024.1 | |
| I-396 | A | B | | | 2103.0 | 1052.6 | 2102.2 | |
| I-397 | A | B | | | 2088.9 | 2111.9 | 2088.1 | |
| I-398 | A | B | | | 2104.9 | 2106.0 | 2104.1 | |
| I-399 | A | A | | | 2190.0 | 1096.0 | 2189.0 | |
| I-400 | A | B | | | 2176.0 | 1089.0 | 2175.0 | |
| I-401 | A | A | | | 2192.0 | 2192.8 | 2191.0 | |
| I-402 | A | B | | | 2174.0 | 2197.0 | 2173.2 | |
| I-403 | A | | | | 2174.0 | 1088.1 | 2173.0 | |
| I-404 | A | A | | | 2160.0 | 2182.8 | 2159.0 | |
| I-405 | A | A | | | 2160.0 | 2182.8 | 2159.0 | |
| I-406 | A | A | | | 2176.0 | 1089.1 | 2075.1 | |
| I-407 | A | B | | | 2188.0 | 2210.9 | 2187.0 | |
| I-408 | A | A | | | 2188.0 | 2210.8 | 2187.0 | |
| I-409 | A | A | | | 2174.0 | 2196.8 | 2173.0 | |
| I-410 | A | | | | 2174.0 | 2197.1 | 2173.3 | |
| I-411 | A | B | | | 2190.0 | 2190.8 | 2188.8 | |
| I-412 | A | A | | | 2190.0 | 2213.1 | 2189.3 | |
| I-413 | A | A | | | 2112.9 | 1057.6 | 2112.0 | |
| I-414 | A | A | | | 2127.0 | 2127.8 | 2126.0 | |
| I-415 | A | A | | | 2114.9 | 2115.8 | 2113.9 | |
| I-416 | A | A | | | 2171.9 | 2172.8 | 2171.0 | |
| I-417 | A | B | | | 2200.0 | 1101.1 | 2199.0 | |
| I-418 | A | B | | | 2079.0 | 2079.8 | 2077.9 | |
| I-419 | A | | | | 2093.0 | 2094.1 | 2092.1 | |
| I-420 | A | B | | | 2093.0 | 2093.8 | 2091.9 | |
| I-421 | A | D | | | 2070.9 | 2071.9 | 2070.0 | |
| I-422 | A | D | | | 2064.9 | 2065.8 | 2063.9 | |
| I-423 | A | C | | | 2084.9 | 2085.8 | 2083.9 | |
| I-424 | A | C | | | 2079.0 | 2079.9 | 2078.0 | |
| I-425 | B | D | | | 2008.9 | 1005.5 | 1003.6 | A |
| I-426 | A | D | | | 2022.9 | 2024.0 | 2022.1 | A |
| I-427 | A | D | | | 2004.8 | 1003.5 | 1001.6 | |
| I-428 | A | D | | | 2006.9 | 2007.8 | 2006.0 | B |
| I-429 | A | D | | | 2018.9 | 1010.5 | 1008.7 | |
| I-430 | B | D | | | 2020.9 | 2022.3 | 2020.4 | |
| I-431 | A | D | | | 2020.9 | 2022.1 | 2020.2 | B |
| I-432 | A | D | | | 2117.0 | 2118.8 | 2116.9 | |
| I-433 | A | C | | | 2117.0 | 1059.8 | 1057.9 | |
| I-434 | A | D | | | 2103.0 | 1052.7 | 1050.9 | |
| I-435 | A | D | | | 2090.9 | 1046.7 | 1044.8 | |
| I-436 | A | C | | | 2090.9 | 2092.0 | 2090.2 | |
| I-437 | A | D | | | 2090.9 | 1046.7 | 1044.9 | |
| I-438 | A | D | | | 2151.0 | 1076.7 | 1074.9 | |
| I-439 | A | D | | | 2088.9 | 1045.8 | 1043.9 | |
| I-440 | A | D | | | 2086.9 | 1044.7 | 1042.8 | |
| I-441 | A | D | | | 2074.9 | 1038.7 | 1036.9 | |
| I-442 | B | D | | | 2074.9 | 1038.7 | 1036.7 | |
| I-443 | A | D | | | 2072.9 | 1037.7 | 1035.8 | |
| I-444 | A | C | | | 2090.9 | 1046.9 | 1044.9 | |
| I-445 | A | C | | | 2104.9 | 1053.7 | 1051.8 | |
| I-446 | A | D | | | 2104.9 | 2106.3 | 2104.5 | |
| I-447 | A | D | | | 2103.0 | 1052.8 | 1050.8 | |
| I-448 | A | D | | | 2103.0 | 1052.8 | 1051.0 | |
| I-449 | A | D | | | 2117.0 | 1059.8 | 1057.9 | |
| I-450 | A | D | | | 2117.0 | 1059.8 | 1057.9 | |
| I-451 | A | D | | | 2131.9 | 1067.2 | 1065.4 | |
| I-452 | A | D | | | 2131.9 | 1067.2 | 1065.3 | |
| I-453 | A | D | | | 2202.0 | 1102.3 | 1100.4 | |
| I-454 | A | D | | | 2132.0 | 2133.5 | 2131.5 | |
| I-455 | A | D | | | 2132.0 | 2133.6 | 2131.5 | |
| I-456 | A | D | | | 2151.0 | 1076.8 | 1074.8 | |
| I-457 | A | C | | | 2151.0 | 1076.7 | 1074.8 | |
| I-458 | A | B | | | 2074.9 | 1038.7 | 2074.3 | |
| I-459 | A | | | | 2074.9 | 2098.0 | 2074.2 | |
| I-460 | A | | | | 2162.0 | 2185.1 | 2161.3 | |
| I-461 | A | C | | | 2162.0 | 1082.3 | 2161.5 | |
| I-462 | A | C | | | 2117.0 | 2140.2 | 2116.3 | |
| I-463 | A | | | | 2204.0 | 2227.1 | 2203.4 | |
| I-464 | A | B | | | 2275.0 | 2298.3 | 2274.7 | |
| I-465 | A | B | | | 2317.1 | 2340.3 | 2316.5 | |
| I-466 | A | | | | 2317.1 | 2340.3 | 2316.5 | |
| I-467 | A | B | | | 2317.1 | 1159.8 | 2316.6 | |
| I-468 | A | C | | | 2317.1 | 1095.3 | 1093.4 | |
| I-469 | B | D | | | 2155.9 | 1070.0 | 1068.1 | |
| I-470 | E | D | | | 2103.0 | 1052.8 | 1050.8 | |
| I-471 | A | D | | | 2082.9 | 2083.7 | 2081.8 | |
| I-472 | A | B | | | 2114.9 | 1059.1 | 2115.3 | |
| I-473 | A | C | | | 2046.9 | 2048.8 | 2047.1 | |
| I-474 | A | C | | | 2046.9 | 1025.1 | 1023.3 | |
| I-475 | A | C | | | 2072.9 | 1037.9 | 2073.1 | |
| I-476 | A | C | | | 2072.9 | 1038.1 | 2073.2 | |
| I-477 | A | C | | | 2096.9 | 2098.7 | 2096.9 | |
| I-478 | A | B | | | 2086.9 | 2088.8 | 2086.8 | |
| I-479 | A | C | | | 2046.9 | 1024.9 | 2047.6 | |
| I-480 | A | D | | | 2046.9 | 1025.1 | 1023.1 | |
| I-481 | A | | | | 2130.9 | 1067.1 | 2131.1 | |
| I-482 | A | | | | 2130.9 | 1067.1 | 1065.1 | |
| I-483 | A | C | | | 2063.0 | 2064.2 | 2062.2 | |
| I-484 | A | D | | | 2063.0 | 2064.2 | 2062.2 | |
| I-485 | A | C | | | 2069.0 | 2070.2 | 2068.1 | |
| I-486 | A | | | | 2069.0 | 2070.2 | 2068.1 | |
| I-487 | A | C | | | 2097.0 | 1049.8 | 2096.6 | |
| I-488 | A | | | | 2097.0 | 1049.8 | 2096.7 | |
| I-489 | A | | | | 2029.0 | 2030.2 | 2028.2 | |
| I-490 | B | | | | 2029.0 | 1015.6 | 2028.2 | |
| I-491 | B | | | | 2055.0 | 1028.6 | 2054.2 | |
| I-492 | B | | | | 2055.0 | 1028.6 | 2054.2 | |
| I-493 | A | C | | | 2303.0 | 1152.9 | 1151.1 | |
| I-494 | A | C | | | 2317.0 | 1159.9 | 1157.9 | |
| I-495 | A | B | | | 2317.0 | 1159.9 | 1158.1 | |
| I-496 | A | C | | | 2275.0 | 1138.9 | 1137.1 | |
| I-497 | A | B | | | 2275.0 | 1138.9 | 1137.1 | |
| I-498 | A | B | | | 2289.1 | 1145.9 | 1144.1 | |
| I-499 | A | C | | | 2289.1 | 1145.9 | 1143.9 | |
| I-500 | A | B | | | 2289.1 | 1145.9 | 1144.1 | |
| I-501 | A | C | | | 2289.1 | 1145.9 | 2287.9 | |
| I-502 | A | C | | | 2345.1 | 1173.9 | 1172.1 | |
| I-503 | A | C | | | 2345.1 | 1174.1 | 1171.9 | |
| I-504 | A | C | | | 2359.1 | 1180.9 | 1179.1 | |
| I-505 | A | C | | | 2359.1 | 1181.1 | 1178.9 | |
| I-506 | A | C | | | 2289.0 | 1145.9 | 1144.1 | |
| I-507 | A | B | | | 2303.0 | 1152.9 | 1151.1 | |
| I-508 | A | B | | | 2275.0 | 1138.9 | 1137.1 | |
| I-509 | A | B | | | 2275.0 | 1138.9 | 1137.1 | |
| I-510 | A | B | | | 2275.0 | 1138.9 | 1136.9 | |
| I-511 | A | B | | | 2275.0 | 1139.1 | 1137.1 | |
| I-512 | A | C | | | 2331.1 | 1166.9 | 1165.1 | |
| I-513 | A | C | | | 2331.1 | 1166.9 | 1164.9 | |
| I-514 | A | C | | | 2132.9 | 2133.5 | 2131.7 | |
| I-515 | A | D | | | 2146.9 | 1074.4 | 1072.5 | |
| I-516 | A | C | | | 2161.0 | 1081.4 | 1079.4 | |
| I-517 | A | D | | | 2781.3 | 1392.1 | 1390.2 | |
| I-518 | E | | | | 2809.3 | 1406.2 | 1404.2 | |
| I-519 | A | D | | | 2137.9 | 2139.3 | 2137.4 | |
| I-520 | A | D | | | 2151.9 | 2153.3 | 2151.4 | |
| I-521 | A | | | | 2118.9 | 2121.1 | 2119.0 | |
| I-522 | C | | | | 2104.9 | 2107.1 | 2105.1 | |
| I-523 | A | | | | 2132.9 | 2135.1 | 2133.1 | |
| I-524 | B | | | | 2118.9 | 2121.1 | 2118.9 | |
| I-525 | A | | | | 2146.9 | 2149.2 | 2146.9 | |
| I-526 | C | | | | 2133.0 | 2135.1 | 2133.2 | |
| I-527 | A | | | | 2117.0 | 2119.2 | 2117.1 | |
| I-528 | A | | | | 2117.0 | 2119.0 | 2117.0 | |
| I-529 | A | | | | 2151.0 | 2153.1 | 2150.8 | |
| I-530 | A | | | | 2165.0 | 2167.2 | 2165.1 | |
| I-531 | A | | | | 2129.0 | 2131.2 | 2129.1 | |
| I-532 | A | | | | 2129.0 | 2131.0 | 2129.1 | |
| I-533 | A | | | | 2181.0 | 2183.1 | 2180.6 | |
| I-534 | A | | | | 2174.0 | 2176.0 | 2174.0 | |
| I-535 | B | | | | 2021.0 | 2022.3 | 2020.5 | |
| I-536 | B | | | | 2035.0 | 2036.5 | 2034.6 | |
| I-537 | A | | | | 2061.0 | 1031.8 | 1029.8 | |
| I-538 | A | | | | 2049.0 | 2050.4 | 2048.5 | |
| I-539 | A | | | | 2049.0 | 2050.4 | 2048.6 | |
| I-540 | C | | | | 2091.0 | 2092.5 | 2090.6 | |
| I-541 | A | | | | 2074.9 | 2076.4 | 2074.6 | |
| I-542 | A | | | | 2112.0 | 2113.3 | 2111.5 | |
| I-543 | B | | | | 2088.9 | 1045.8 | 1043.9 | |
| I-544 | C | | | | 2088.9 | 1045.8 | 1043.8 | |
| I-545 | C | D | | | 2103.0 | 2104.5 | 2102.5 | |
| I-546 | C | | | | 2103.0 | 1052.8 | 1050.9 | |
| I-547 | A | D | | | 2088.9 | 2090.4 | 2088.2 | |
| I-548 | C | D | | | 2103.0 | 2104.4 | 2102.5 | |
| I-549 | E | D | | | 2117.0 | 2118.4 | 2116.6 | |
| I-550 | E | D | | | 2117.0 | 2118.4 | 2116.6 | |
| I-551 | E | D | | | 2131.0 | 2132.5 | 2130.6 | |
| I-552 | E | | | | 2040.9 | 2042.4 | 2040.5 | |
| I-553 | E | D | | | 2055.0 | 2056.6 | 2054.7 | |
| I-554 | E | D | | | 2026.9 | 2028.4 | 2026.6 | |
| I-555 | E | D | | | 2040.9 | 2042.3 | 2040.5 | |
| I-556 | E | D | | | 2069.0 | 2070.5 | 2068.6 | |
| I-557 | A | | | | 2074.9 | 2077.1 | 2075.0 | |
| I-558 | A | | | | 2060.9 | 2063.0 | 2060.9 | |
| I-559 | A | | | | 2046.9 | 2049.0 | 2047.2 | |
| I-560 | A | | | | 2074.9 | 2077.1 | 2074.8 | |
| I-561 | A | | | | 2088.9 | 2091.1 | 2089.1 | |
| I-562 | B | D | | | 2127.9 | 2129.1 | 2127.2 | A |
| I-563 | A | D | | | 2141.9 | 2143.0 | 2141.2 | A |
| I-564 | B | D | | | 2136.0 | 1069.2 | 1067.3 | A |
| I-565 | A | D | | | 2162.0 | 2163.1 | 2161.3 | A |
| I-566 | B | | | | 2193.9 | 1198.3 | 1196.3 | |
| I-567 | B | | | | 2219.9 | 1111.4 | 1109.3 | |
| I-568 | A | | | | 2092.9 | 2095.1 | 2093.1 | |
| I-569 | A | | | | 2092.9 | 2094.8 | 2092.6 | |
| I-570 | A | | | | 2108.9 | 2111.5 | 2110.0 | |
| I-571 | A | | | | 2108.9 | 2111.8 | 2109.9 | |
| I-572 | A | | | | 2152.8 | 2156.1 | 2153.8 | |
| I-573 | A | | | | 2152.8 | 2155.9 | 2153.7 | |
| I-574 | A | | | | 2092.9 | 2095.0 | 2092.8 | |
| I-575 | A | | | | 2092.9 | 2094.9 | 2092.6 | |
| I-576 | A | | | | 2108.9 | 2111.5 | 2110.0 | |
| I-577 | A | | | | 2108.9 | 1056.5 | 1054.8 | |
| I-578 | A | | | | 2152.8 | 2156.0 | 2153.8 | |
| I-579 | A | | | | 2152.8 | 2155.8 | 2154.4 | |
| I-580 | A | | | | 2092.9 | 2095.2 | 2093.3 | |
| I-581 | A | | | | 2092.9 | 2095.0 | 2093.2 | |
| I-582 | A | | | | 2104.9 | 1054.2 | 1052.4 | |
| I-583 | A | | | | 2104.9 | 2106.9 | 2104.8 | |
| I-584 | A | | | | 2099.9 | 2101.9 | 2100.1 | |
| I-585 | A | | | | 2099.9 | 2101.9 | 2099.8 | |
| I-586 | A | | | | 2099.9 | 2101.9 | 2099.8 | |
| I-587 | A | | | | 2099.9 | 2101.9 | 2100.0 | |
| I-588 | A | | | | 2088.9 | 2091.1 | 2088.8 | |
| I-589 | A | | | | 2088.9 | 2090.9 | 2089.0 | |
| I-590 | A | | | | 2088.9 | 2091.1 | 2089.6 | |
| I-591 | A | | | | 2088.9 | 2091.1 | 2088.8 | |
| I-592 | E | | | | 2086.9 | 2088.8 | 2087.1 | |
| I-593 | E | | | | 2086.9 | 2088.9 | 2087.0 | |
| I-594 | E | | | | 2117.0 | 2119.2 | 2117.2 | |
| I-595 | E | | | | 2117.0 | 2119.0 | 2117.1 | |
| I-596 | E | | | | 2117.0 | 2119.1 | 2116.8 | |
| I-597 | E | | | | 2117.0 | 2119.1 | 2117.0 | |
| I-598 | E | | | | 2117.0 | 2119.2 | 2117.3 | |
| I-599 | E | | | | 2117.0 | 2119.2 | 2116.9 | |
| I-600 | E | | | | 2117.0 | 2119.2 | 2117.1 | |
| I-601 | E | | | | 2117.0 | 2119.0 | 2117.0 | |
| I-602 | E | | | | 2117.0 | 2119.1 | 2116.9 | |
| I-603 | E | | | | 2117.0 | 2119.0 | 2117.0 | |
| I-604 | E | | | | 2117.0 | 2119.1 | 2116.9 | |
| I-605 | E | | | | 2117.0 | 2119.0 | 2117.2 | |
| I-606 | E | | | | 2186.0 | 2188.1 | 2186.2 | |
| I-607 | A | | | | 2158.0 | 2160.2 | 2158.1 | |

Structural information and compositions of stapled peptides are described in Table E2.
1. Compound ID
2. beta-Catenin FP IC50 (nM): A ≤ 50 nM; 50 nM < B ≤ 200 nM; 200 nM < C ≤ 750 nM; 750 nM < D ≤ 1000 nM; E > 1000 nM
3. NanoBRET Abs IC50 (uM): A ≤ 1.5 uM; 1.5 uM < B ≤ 3.0 uM; 3.0 uM < C ≤ 10.0 uM; D > 10.0 uM
4. DLD1 4% Abs IC50 (uM): “+” ≤ 1.0 uM; 1.0 uM < “++” ≤ 5.0 uM; “+++” > 5.0 uM
5. COLO320DM Proliferation Abs IC50 (uM): “+” ≤ 10.0 uM; 10.0 uM < “++” ≤ 20.0 uM; “+++” > 20.0 uM
6. Calculated Mass
7. Found m/z (positive mode)
8. Found m/z (negative mode)
9. C═C double bond (e.g., —CH═CH—) reduction to single bond (e.g., —CH₂—CH₂—). A: —CH═CH— in each staple reduced to —CH₂—CH₂—; B: —CH═CH— in C-terminal side staple reduced to —CH₂—CH₂— (see, e.g., see preparation of I-428 and I-432 as examples)

Table E2. Certain peptides and compositions thereof as examples. Peptides are stapled unless indicated otherwise (among other things, the present disclosure also provides unstapled versions of such peptides, optionally protected with one or more protection group (e.g., protection of N-terminus, C-terminus, side chains, etc.), and intermediates thereof). As appreciated by those skilled in the art, stapling may provide more than one stereoisomers (e.g., E/Z of double bonds and/or diastereomers).

In some embodiments, a double bond in a staple is E. In some embodiments, a double bond in a staple is Z. In some embodiments, isomers (or combinations thereof) are listed separately (typically based on reverse phase HPLC peaks (e.g., detected by UV (e.g., at 220 nm) and/or MS) in the order of elution: each earlier eluted peak is assigned a smaller ID number than each later eluted peaks (if any); in some cases, a peak may contain two or more isomers; in some cases, isomers are not separated (or single isomer), e.g., when there is one peak on HPLC). Compositions utilized in various assays are typically of stapled peptides; the present disclosure also provides peptides prior to stapling and compositions thereof. A general HPLC method: Xselect CSH C₁₈ column 1.7 um 2.1×50 mm 130 Å; Column temperature 40° C.; Flow 0.6 mL/min; 0.1% formic acid in both acetonitrile and water, 7.2 min gradient from 5 to 95% acetonitrile. In some embodiments, a different gradient and/or a C8 column were used.

| 1 | Description |
|---|---|
| I-1 | Ac-PL3-Asp-Leu-B5-Asp-Asp-Ala-Ala-Phe-dLys*3-PyrS2-3Thi-BztA-GlnR*3-NH2 |
| I-2 | Ac-PL3-Asp-Leu-B5-Asp-Asp-Ala-Ala-Phe-dLys*3-PyrS2-3Thi-BztA-GlnR*3-NH2 |
| I-3 | Ac-PL3-Asp-Leu-B5-Asp-Asp-Ala-Ala-Phe-Lys*3-PyrS2-3Thi-BztA-GlnR*3-NH2 |
| I-4 | Ac-PL3-Asp-Leu-B5-Asp-Asp-Ala-Ala-Phe-Lys*3-PyrS2-3Thi-BztA-GlnR*3-NH2 |
| I-5 | Ac-PL3-Asp-Leu-B5-Asp-Asp-Ala-Ala-Phe-DGlnR*3-PyrS2-3Thi-BztA-Lys*3-NH2 |
| I-6 | Ac-PL3-Asp-Leu-B5-Asp-Asp-Ala-Ala-Phe-GlnR*3-PyrS2-3Thi-BztA-Lys*3-NH2 |
| I-7 | Ac-PL3-Asp-Leu-B5-Asp-Asp-Lys*3-Ala-Phe-GlnR*3-PyrS2-3Thi-BztA-Gln-NH2 |
| I-8 | Ac-PL3-Asp-Leu-B5-Asp-Asp-GlnR*3-Ala-Phe-Lys*3-PyrS2-3Thi-BztA-Gln-NH2 |
| I-9 | Ac-PL3-Asp-Leu-B5-Asp-3COOHF-Ala-Ala-Phe-dLys*3-PyrS2-3Thi-BztA-GlnR*3-NH2 |
| I-10 | Ac-PL3-Asp-Leu-B5-Asp-3COOHF-Ala-Ala-Phe-dLys*3-PyrS2-3Thi-BztA-GlnR*3-NH2 |
| I-11 | Ac-PL3-Asp-Leu-B5-Asp-3COOHF-Ala-Ala-Phe-Lys*3-PyrS2-3Thi-BztA-GlnR*3-NH2 |
| I-12 | Ac-PL3-Asp-Leu-B5-Asp-3COOHF-Ala-Ala-Phe-Lys*3-PyrS2-3Thi-BztA-GlnR*3-NH2 |
| I-13 | Ac-PL3-Asp-Leu-B5-Asp-3COOHF-Ala-Ala-Phe-DGlnR*3-PyrS2-3Thi-BztA-Lys*3-NH2 |
| I-14 | Ac-PL3-Asp-Leu-B5-Asp-3COOHF-Ala-Ala-Phe-DGlnR*3-PyrS2-3Thi-BztA-Lys*3-NH2 |
| I-15 | Ac-PL3-Asp-Leu-B5-Asp-3COOHF-Ala-Ala-Phe-GlnR*3-PyrS2-3Thi-BztA-Lys*3-NH2 |
| I-16 | Ac-PL3-Asp-Leu-B5-Asp-3COOHF-Ala-Ala-Phe-GlnR*3-PyrS2-3Thi-BztA-Lys*3-NH2 |
| I-17 | Ac-PL3-Asp-Npg-B5-Asp-Asp-Ala-Ala-Phe-TriAzLys*3-PyrS2-3Thi-BztA-sAla*3-NH2 |
| I-18 | Ac-PL3-Asp-Npg-B5-Asp-Asp-Ala-Ala-Phe-TriAzLys*3-PyrS2-3Thi-BztA-sAla*3-NH2 |
| I-19 | Ac-PL3-Asp-Npg-B5-Asp-3COOHF-Ala-Ala-Phe-TriAzLys*3-PyrS2-3Thi-BztA-sAla*3-NH2 |
| I-20 | Ac-PL3-Asp-Npg-B5-Asp-3COOHF-Ala-Ala-Phe-TriAzLys*3-PyrS2-3Thi-BztA-sAla*3-NH2 |
| I-21 | Ac-PL3-Asp-Npg-B5-Asp-3COOHF-Ala-Ala-Phe-TriAzLys*3-PyrS2-2F3MeF-BztA-sAla*3- |
| | NH2 |
| I-22 | Ac-PL3-Asp-Npg-B5-Asp-3COOHF-Ala-Ala-Phe-TriAzLys*3-PyrS2-2F3MeF-BztA-sAla*3- |
| | NH2 |
| I-23 | Ac-PL3-Asp-Npg-B5-Asp-Asp-Ala-Ala-Phe-sAla*3-PyrS2-3Thi-BztA-TriAzLys*3-NH2 |
| I-24 | Ac-PL3-Asp-Npg-B5-Asp-3COOHF-Ala-Ala-Phe-sAla*3-PyrS2-3Thi-BztA-TriAzLys*3-NH2 |
| I-25 | Ac-PL3-Asp-Npg-B5-Asp-3COOHF-Ala-Ala-Phe-sAla*3-PyrS2-2F3MeF-BztA-TriAzLys*3- |
| | NH2 |
| I-26 | Ac-PL3-Asp-Npg-B5-Asp-Asp-sAla*3-Ala-Phe-TriAzLys*3-PyrS2-3Thi-BztA-Gln-NH2 |
| I-27 | Ac-PL3-Asp-Npg-B5-Asp-3COOHF-sAla*3-Ala-Phe-TriAzLys*3-PyrS2-3Thi-BztA-Gln-NH2 |
| I-28 | Ac-PL3-Asp-Npg-B5-Asp-3COOHF-sAla*3-Ala-Phe-TriAzLys*3-PyrS2-2F3MeF-BztA-Gln- |
| | NH2 |
| I-29 | Ac-PL3-Asp-Npg-B5-Asp-Asp-TriAzLys*3-Ala-Phe-sAla*3-PyrS2-3Thi-BztA-Gln-NH2 |
| I-30 | Ac-PL3-Asp-Npg-B5-Asp-3COOHF-TriAzLys*3-Ala-Phe-sAla*3-PyrS2-3Thi-BztA-Gln-NH2 |
| I-31 | Ac-PL3-Asp-Npg-B5-Asp-3COOHF-TriAzLys*3-Ala-Phe-sAla*3-PyrS2-2F3MeF-BztA-Gln- |
| | NH2 |
| I-32 | Ac-PL3-Asp-Npg-B5-Asp-3COOHF-Ala-Ala-Phe-Lys*3-PyrS2-3Thi-BztA-GlnR*3-NH2 |
| I-33 | Ac-PL3-Asp-Npg-B5-Asp-3COOHF-Ala-Ala-Phe-GlnR*3-PyrS2-3Thi-BztA-Lys*3-NH2 |
| I-34 | Ac-PL3-Asp-Npg-B5-Asp-3COOHF-Ala-Ala-Phe-Lys*3-PyrS2-3Thi-BztA-AsnR*3-NH2 |
| I-35 | Ac-PL3-Asp-Npg-B5-Asp-3COOHF-Ala-Ala-Phe-AsnR*3-PyrS2-3Thi-BztA-Lys*3-NH2 |
| I-36 | Ac-PL3-Asp-Npg-B5-Asp-3COOHF-Ala-Ala-Phe-AsnR*3-PyrS2-3Thi-BztA-Lys*3-NH2 |
| I-37 | Ac-PL3-Asp-Npg-B5-Asp-3COOHF-Ala-Ala-Phe-Orn*3-PyrS2-3Thi-BztA-GlnR*3-NH2 |
| I-38 | Ac-PL3-Asp-Npg-B5-Asp-3COOHF-Ala-Ala-Phe-GlnR*3-PyrS2-3Thi-BztA-Orn*3-NH2 |
| I-39 | Ac-PL3-Asp-Npg-B5-Asp-3COOHF-Ala-Ala-Phe-Orn*3-PyrS2-3Thi-BztA-AsnR*3-NH2 |
| I-40 | Ac-PL3-Asp-Npg-B5-Asp-3COOHF-Ala-Ala-Phe-AsnR*3-PyrS2-3Thi-BztA-Orn*3-NH2 |
| I-41 | Ac-PL3-Asp-Npg-B5-Asp-3COOHF-Ala-Ala-Phe-AsnR*3-PyrS2-3Thi-BztA-Orn*3-NH2 |
| I-42 | Ac-PL3-Asp-Npg-B5-Asp-3COOHF-Ala-Ala-Phe-Lys*3-PyrS2-3Thi-BztA-GlnR*3-Ala-NH2 |
| I-43 | Ac-PL3-Asp-Npg-B5-Asp-3COOHF-Ala-Ala-Phe-GlnR*3-PyrS2-3Thi-BztA-Lys*3-Ala-NH2 |
| I-44 | Ac-PL3-Asp-Npg-B5-Asp-3COOHF-Ala-Ala-Phe-AsnR*3-PyrS2-3Thi-BztA-Lys*3-Ala-NH2 |
| I-45 | Ac-PL3-Asp-Npg-B5-Asp-3COOHF-[isophthalate]-Lys-Ala-Phe-Leu-PyrS2-3Thi-BztA-Lys-NH2 |
| I-46 | Ac-PL3-Asp-Npg-B5-Asp-3COOHF-[succinate]-Lys-Ala-Phe-Leu-PyrS2-3Thi-BztA-Lys-NH2 |
| I-47 | Ac-PL3-Asp-Npg-B5-Asp-3COOHF-[Me2Mal]-Lys-Ala-Phe-Leu-PyrS2-3Thi-BztA-Lys-NH2 |
| I-48 | Ac-PL3-Asp-Npg-B5-Asp-3COOHF-[diphenate]-Lys-Ala-Phe-Leu-PyrS2-3Thi-BztA-Lys-NH2 |
| I-49 | Ac-PL3-Asp-Npg-B5-Asp-3COOHF-[Biphen33COOH]-Lys-Ala-Phe-Leu-PyrS2-3Thi-BztA-Lys- |
| | NH2 |
| I-50 | Ac-PL3-Asp-Npg-B5-Asp-3COOHF-[isophthalate]-Lys-Ala-Phe-Leu-PyrS2-2ClF-BztA-Lys-NH2 |
| I-51 | Ac-PL3-Asp-Npg-B5-Asp-3COOHF-[succinate]-Lys-Ala-Phe-Leu-PyrS2-2ClF-BztA-Lys-NH2 |
| I-52 | Ac-PL3-Asp-Npg-B5-Asp-3COOHF-[Me2Mal]Lys-Ala-Phe-Leu-PyrS2-2ClF-BztA-Lys-NH2 |
| I-53 | Ac-PL3-Asp-Npg-B5-Asp-3COOHF-[diphenate]Lys-Ala-Phe-Leu-PyrS2-2ClF-BztA-Lys-NH2 |
| I-54 | Ac-PL3-Asp-Npg-B5-Asp-3COOHF-[Biphen33COOH]Lys-Ala-Phe-Leu-PyrS2-2ClF-BztA-Lys- |
| | NH2 |
| I-55 | Ac-PL3-Asp-Npg-B5-Asp-3COOHF-Ala-Ala-Phe-Lys*3-PyrS2-2F3MeF-BztA-GlnR*3-NH2 |
| I-56 | Ac-PL3-Asp-Npg-B5-Asp-3COOHF-Ala-Ala-Phe-Lys*3-PyrS2-2F3MeF-BztA-AsnR*3-NH2 |
| I-57 | Ac-PL3-Asp-Npg-B5-Asp-3COOHF-Ala-Ala-Phe-Lys*3-PyrS2-2F3MeF-BztA-GlnR*3-Ala-NH2 |
| I-58 | Ac-PL3-Asp-Npg-B5-Asp-3COOHF-Ala-Ala-Phe-Lys*3-PyrS2-2F3MeF-BztA-AsnR*3-Ala- |
| | NH2 |
| I-59 | Ac-PL3-Asp-Npg-B5-Asp-3COOHF-Ala-Ala-Phe-Lys*3-PyrS2-2F3MeF-BztA-AsnR*3-Ala- |
| | NH2 |
| I-60 | Ac-PL3-Asp-Npg-B5-Asp-3COOHF-Gln-Ala-Phe-Lys*3-PyrS2-2F3MeF-BztA-GlnR*3-Ala-NH2 |
| I-61 | Ac-PL3-Asp-Npg-B5-Asp-3COOHF-Gln-Ala-Phe-Lys*3-PyrS2-2F3MeF-BztA-AsnR*3-Ala- |
| | NH2 |
| I-62 | Ac-PL3-Asp-Npg-B5-Asp-3COOHF-Ala-Ala-Phe-Lys*3-PyrS2-3Thi-BztA-GlnR*3-Throl |
| I-63 | Ac-PL3-Asp-Npg-B5-Asp-3COOHF-Ala-Ala-Phe-Lys*3-PyrS2-3Thi-BztA-GlnR*3-Throl |
| I-64 | Ac-PL3-Asp-Npg-B5-Asp-3COOHF-Ala-Ala-Phe-Lys*3-PyrS2-3Thi-BztA-GlnR*3-Prool |
| I-65 | Ac-PL3-Asp-Npg-B5-Asp-3COOHF-Ala-Ala-Phe-Lys*3-PyrS2-3Thi-BztA-GlnR*3-Alaol |
| I-66 | Ac-PL3-Asp-Npg-B5-Asp-3COOHF-Aib-Ala-Phe-Lys*3-PyrS2-3Thi-BztA-GlnR*3-Ala-NH2 |
| I-67 | Ac-PL3-Asp-Npg-B5-Asp-3COOHF-Aib-Ala-Phe-Lys*3-PyrS2-3Thi-BztA-GlnR*3-Ala-NH2 |
| I-68 | Ac-PL3-Asp-Npg-B5-Asp-3COOHF-Aib-Ala-Phe-Lys*3-PyrS2-2F3MeF-BztA-GlnR*3-Ala-NH2 |
| I-69 | Ac-PL3-Asp-Npg-B5-Asp-3COOHF-Aib-Ala-Phe-Lys*3-PyrS2-2F3MeF-BztA-GlnR*3-Ala-NH2 |
| I-70 | Ac-PL3-Asp-Npg-B5-Asp-3COOHF-nLeu-Ala-Phe-Lys*3-PyrS2-3Thi-BztA-GlnR*3-Ala-NH2 |
| I-71 | Ac-PL3-Asp-Npg-B5-Asp-3COOHF-nLeu-Ala-Phe-Lys*3-PyrS2-2F3MeF-BztA-GlnR*3-Ala- |
| | NH2 |
| I-72 | Ac-PL3-Asp-Npg-B5-Asp-3COOHF-Thr-Ala-Phe-Lys*3-PyrS2-3Thi-BztA-GlnR*3-Ala-NH2 |
| I-73 | Ac-PL3-Asp-Npg-B5-Asp-3COOHF-Thr-Ala-Phe-Lys*3-PyrS2-2F3MeF-BztA-GlnR*3-Ala-NH2 |
| I-74 | Ac-PL3-Asp-GlnR**3-B5-Asp-3COOHF-Lys**3-Ala-Phe-Lys*3-PyrS2-3Thi-BztA-GlnR*3-Ala- |
| | NH2 |
| I-75 | Ac-PL3-Asp-GlnR**3-B5-Asp-3COOHF-Lys**3-Ala-Phe-Lys*3-PyrS2-2F3MeF-BztA-GlnR*3- |
| | Ala-NH2 |
| I-76 | Ac-PL3-Asp-Npg-B5-Asp-3COOHF-Ala-Ala-Phe-Lys*3-PyrS2-3Thi-BztA-GlnR*3-Ala-NH2 |
| I-77 | Ac-PL3-Asp-Npg-B5-Asp-3COOHF-Ala-Ala-Phe-Lys*3-PyrS2-2F3MeF-BztA-GlnR*3-Ala-NH2 |
| I-78 | Ac-PL3-Asp-GlnR**3-B5-Asp-3COOHF-Lys**3-Ala-Phe-Lys*3-PyrS2-3Thi-BztA-GlnR*3-Ala- |
| | NH2 |
| I-79 | Ac-PL3-Asp-GlnR**3-B5-Asp-3COOHF-Lys**3-Ala-Phe-Lys*3-PyrS2-2F3MeF-BztA-GlnR*3- |
| | Ala-NH2 |
| I-80 | Ac-PL3-Asn-Npg-B5-Asp-3COOHF-Ala-Ala-Phe-Lys*3-PyrS2-3Thi-BztA-GlnR*3-Ala-NH2 |
| I-81 | Ac-PL3-Hse-Npg-B5-Asp-3COOHF-Ala-Ala-Phe-Lys*3-PyrS2-3Thi-BztA-GlnR*3-Ala-NH2 |
| I-82 | Ac-PL3-Asp-Npg-B5-Asn-3COOHF-Ala-Ala-Phe-Lys*3-PyrS2-3Thi-BztA-GlnR*3-Ala-NH2 |
| I-83 | Ac-PL3-Asp-Npg-B5-Hse-3COOHF-Ala-Ala-Phe-Lys*3-PyrS2-3Thi-BztA-GlnR*3-Ala-NH2 |
| I-84 | Ac-PL3-Asn-Npg-B5-Asp-3COOHF-Ser-Ala-Phe-Lys*3-PyrS2-3Thi-BztA-GlnR*3-Ala-NH2 |
| I-85 | Ac-PL3-Hse-Npg-B5-Asp-3COOHF-Ser-Ala-Phe-Lys*3-PyrS2-3Thi-BztA-GlnR*3-Ala-NH2 |
| I-86 | Ac-PL3-Asp-Npg-B5-Asn-3COOHF-Ser-Ala-Phe-Lys*3-PyrS2-3Thi-BztA-GlnR*3-Ala-NH2 |
| I-87 | Ac-PL3-Asp-Npg-B5-Hse-3COOHF-Ser-Ala-Phe-Lys*3-PyrS2-3Thi-BztA-GlnR*3-Ala-NH2 |
| I-88 | Ac-PL3-Asn-Npg-B5-Asp-3COOHF-Gln-Ala-Phe-Lys*3-PyrS2-3Thi-BztA-GlnR*3-Ala-NH2 |
| I-89 | Ac-PL3-Hse-Npg-B5-Asp-3COOHF-Gln-Ala-Phe-Lys*3-PyrS2-3Thi-BztA-GlnR*3-Ala-NH2 |
| I-90 | Ac-PL3-Asp-Npg-B5-Asn-3COOHF-Gln-Ala-Phe-Lys*3-PyrS2-3Thi-BztA-GlnR*3-Ala-NH2 |
| I-91 | Ac-PL3-Asp-Npg-B5-Hse-3COOHF-Gln-Ala-Phe-Lys*3-PyrS2-3Thi-BztA-GlnR*3-Ala-NH2 |
| I-92 | Ac-PL3-Asp-Npg-B5-Asp-3COOHF-GlnR-Ala-Phe-Leu-PyrS2-2F3MeF-BztA- |
| | [diaminobutane]GlnR-Ala-NH2 |
| I-93 | Ac-PL3-Asp-Npg-B5-Asp-3COOHF-GlnR-Ala-Phe-Leu-PyrS2-2F3MeF-BztA- |
| | [4aminopiperidine]GlnR-Ala-NH2 |
| I-94 | Ac-PL3-Asp-Npg-B5-Asp-3COOHF-[4aminopiperidine]GlnR-Ala-Phe-Leu-PyrS2-2F3MeF- |
| | BztA-GlnR-Ala-NH2 |
| I-95 | Ac-PL3-Asp-Npg-B5-Asp-3COOHF-GlnR-Ala-Phe-Leu-PyrS2-2F3MeF-BztA- |
| | [4mampiperidine]GlnR-Ala-NH2 |
| I-96 | Ac-PL3-Asp-Npg-B5-Asp-3COOHF-GlnR-Ala-Phe-Leu-PyrS2-2F3MeF-BztA- |
| | [4mampiperidine]GlnR-Ala-NH2 |
| I-97 | Ac-PL3-Asp-Cha-B5-Asp-3COOHF-Ala-Ala-Phe-Lys*3-PyrS2-2F3MeF-BztA-GlnR*3-Ala-NH2 |
| I-98 | Ac-PL3-Asp-Cha-B5-Asp-3COOHF-Ala-Ala-Phe-Lys*3-PyrS2-2F3MeF-BztA-GlnR*3-Ala-NH2 |
| I-99 | Ac-PL3-Asp-Npg-B5-Asp-3COOHF-Npg-Ala-Phe-Lys*3-PyrS2-2F3MeF-BztA-GlnR*3-Ala- |
| | NH2 |
| I-100 | Ac-PL3-Asp-Npg-B5-Asp-3COOHF-Npg-Ala-Phe-Lys*3-PyrS2-2F3MeF-BztA-GlnR*3-Ala- |
| | NH2 |
| I-101 | Ac-PL3-Asp-Npg-B5-Asp-3COOHF-Cha-Ala-Phe-Lys*3-PyrS2-2F3MeF-BztA-GlnR*3-Ala- |
| | NH2 |
| I-102 | Ac-PL3-Asp-Npg-B5-Asp-3COOHF-Cha-Ala-Phe-Lys*3-PyrS2-2F3MeF-BztA-GlnR*3-Ala- |
| | NH2 |
| I-103 | Ac-PL3-Asp-Cha-B5-Asp-3COOHF-Cha-Ala-Phe-Lys*3-PyrS2-2F3MeF-BztA-GlnR*3-Ala-NH2 |
| I-104 | Ac-PL3-Asp-Npg-B5-Asp-3COOHF-Ala-Ala-Phe-Lys*3-PyrS2-2F3MeF-BztA-GlnR*3-Leu- |
| | NH2 |
| I-105 | Ac-PL3-Asp-Npg-B5-Asp-3COOHF-Ala-Ala-Phe-Lys*3-PyrS2-2F3MeF-BztA-GlnR*3-Leu- |
| | NH2 |
| I-106 | Ac-PL3-Asp-Cha-B5-Asp-3COOHF-Ala-Ala-Phe-Lys*3-PyrS2-2F3MeF-BztA-GlnR*3-Leu-NH2 |
| I-107 | Ac-PL3-Asp-Cha-B5-Asp-3COOHF-Ala-Ala-Phe-Lys*3-PyrS2-2F3MeF-BztA-GlnR*3-Leu-NH2 |
| I-108 | Ac-PL3-Asp-Npg-B5-Asp-3COOHF-Npg-Ala-Phe-Lys*3-PyrS2-2F3MeF-BztA-GlnR*3-Leu- |
| | NH2 |
| I-109 | Ac-PL3-Asp-Npg-B5-Asp-3COOHF-Npg-Ala-Phe-Lys*3-PyrS2-2F3MeF-BztA-GlnR*3-Leu- |
| | NH2 |
| I-110 | Ac-PL3-Asp-Npg-B5-Asp-3COOHF-Cha-Ala-Phe-Lys*3-PyrS2-2F3MeF-BztA-GlnR*3-Leu- |
| | NH2 |
| I-111 | Ac-PL3-Asp-Npg-B5-Asp-3COOHF-Cha-Ala-Phe-Lys*3-PyrS2-2F3MeF-BztA-GlnR*3-Leu- |
| | NH2 |
| I-112 | Ac-PL3-Asp-Npg-B5-Asp-TfeGA-Ala-Ala-Phe-Lys*3-PyrS2-2F3MeF-BztA-GlnR*3-Ala-NH2 |
| I-113 | Ac-PL3-Asn-Npg-B5-Asp-TfeGA-Ala-Ala-Phe-Lys*3-PyrS2-2F3MeF-BztA-GlnR*3-Ala-NH2 |
| I-114 | Ac-PL3-Asn-Npg-B5-Asp-TfeGA-Ala-Ala-Phe-Lys*3-PyrS2-2F3MeF-BztA-GlnR*3-Ala-NH2 |
| I-115 | Ac-PL3-Hse-Npg-B5-Asp-TfeGA-Ala-Ala-Phe-Lys*3-PyrS2-2F3MeF-BztA-GlnR*3-Ala-NH2 |
| I-116 | Ac-PL3-Hse-Npg-B5-Asp-TfeGA-Ala-Ala-Phe-Lys*3-PyrS2-2F3MeF-BztA-GlnR*3-Ala-NH2 |
| I-117 | Ac-PL3-Asp-Npg-B5-Asn-TfeGA-Ala-Ala-Phe-Lys*3-PyrS2-2F3MeF-BztA-GlnR*3-Ala-NH2 |
| I-118 | Ac-PL3-Asp-Npg-B5-Hse-TfeGA-Ala-Ala-Phe-Lys*3-PyrS2-2F3MeF-BztA-GlnR*3-Ala-NH2 |
| I-119 | Ac-PL3-Asp-Npg-B5-Hse-TfeGA-Ala-Ala-Phe-Lys*3-PyrS2-2F3MeF-BztA-GlnR*3-Ala-NH2 |
| I-120 | Ac-PL3-Asp-Npg-B5-Asp-TfeGA-Aib-Ala-Phe-Lys*3-PyrS2-2F3MeF-BztA-GlnR*3-Ala-NH2 |
| I-121 | Ac-PL3-Asp-Npg-B5-Asp-TfeGA-Aib-Ala-Phe-Lys*3-PyrS2-2F3MeF-BztA-GlnR*3-Ala-NH2 |
| I-122 | Ac-PL3-Asp-Npg-B5-Asp-TfeGA-Leu-Ala-Phe-Lys*3-PyrS2-2F3MeF-BztA-GlnR*3-Ala-NH2 |
| I-123 | Ac-PL3-Asp-Npg-B5-Asp-TfeGA-Phe-Ala-Phe-Lys*3-PyrS2-2F3MeF-BztA-GlnR*3-Ala-NH2 |
| I-124 | Ac-PL3-Asp-Npg-B5-Asp-TfeGA-Val-Ala-Phe-Lys*3-PyrS2-2F3MeF-BztA-GlnR*3-Ala-NH2 |
| I-125 | Ac-PL3-Asp-Npg-B5-Asp-TfeGA-Val-Ala-Phe-Lys*3-PyrS2-2F3MeF-BztA-GlnR*3-Ala-NH2 |
| I-126 | Ac-PL3-Asp-Npg-B5-Asp-TfeGA-Ala-Ala-Phe-Lys*3-PyrS2-2F3MeF-BztA-GlnR*3-Aib-NH2 |
| I-127 | Ac-PL3-Asp-Npg-B5-Asp-TfeGA-Ala-Ala-Phe-Lys*3-PyrS2-2F3MeF-BztA-GlnR*3-Aib-NH2 |
| I-128 | Ac-PL3-Asp-Npg-B5-Asp-TfeGA-Ala-Ala-Phe-Lys*3-PyrS2-2F3MeF-BztA-GlnR*3-Leu-NH2 |
| I-129 | Ac-PL3-Asp-Npg-B5-Asp-TfeGA-Ala-Ala-Phe-Lys*3-PyrS2-2F3MeF-BztA-GlnR*3-Val-NH2 |
| I-130 | Ac-PL3-Asp-Npg-B5-Asp-3COOHF-GlnR-Ala-Phe-Leu-PyrS2-2F3MeF-BztA- |
| | [39N2spiroundecane]GlnR-Ala-NH2 |
| I-131 | Ac-PL3-Asp-Npg-B5-Asp-3COOHF-GlnR-Ala-Phe-Leu-PyrS2-2F3MeF-BztA- |
| | [29N2spiroundecane]GlnR-Ala-NH2 |
| I-132 | Ac-PL3-Asp-Npg-B5-Asp-3COOHF-[29N2spiroundecane]GlnR-Ala-Phe-Leu-PyrS2-2F3MeF- |
| | BztA-GlnR-Ala-NH2 |
| I-133 | Ac-PL3-Asp-Npg-B5-Asp-3COOHF-GlnR-Ala-Phe-Leu-PyrS2-2F3MeF-BztA- |
| | [39N2spiroundecane]GlnR-Ala-NH2 |
| I-134 | Ac-PL3-Asp-Npg-B5-Asp-3COOHF-GlnR-Ala-Phe-Leu-PyrS2-2F3MeF-BztA- |
| | [29N2spiroundecane]GlnR-Ala-NH2 |
| I-135 | Ac-PL3-Asp-Npg-B5-Asp-3COOHF-[29N2spiroundecane]GlnR-Ala-Phe-Leu-PyrS2-2F3MeF- |
| | BztA-GlnR-Ala-NH2 |
| I-136 | Ac-PL3-Asp-Npg-B5-Asp-3COOHF-Ala-Ala-Phe-Lys*3-PyrS2-2F3MeF-BztA-GlnR*3-Leu-Ser- |
| | NH2 |
| I-137 | Ac-PL3-Asp-Npg-B5-Asp-3COOHF-nLeu-Ala-Phe-Lys*3-PyrS2-2F3MeF-BztA-GlnR*3-Leu- |
| | Ser-NH2 |
| I-138 | Ac-PL3-Asp-Npg-B5-Asp-3COOHF-Aib-Ala-Phe-Lys*3-PyrS2-2F3MeF-BztA-GlnR*3-Leu-Ser- |
| | NH2 |
| I-139 | Ac-PL3-Asp-Npg-B5-Asp-3COOHF-Cpg-Ala-Phe-Lys*3-PyrS2-2F3MeF-BztA-GlnR*3-Leu-Ser- |
| | NH2 |
| I-140 | Ac-PL3-Asp-Npg-B5-Asp-3COOHF-Cbg-Ala-Phe-Lys*3-PyrS2-2F3MeF-BztA-GlnR*3-Leu-Ser- |
| | NH2 |
| I-141 | Ac-PL3-Asp-Npg-B5-Asp-3COOHF-CyLeu-Ala-Phe-Lys*3-PyrS2-2F3MeF-BztA-GlnR*3-Leu- |
| | Ser-NH2 |
| I-142 | Ac-PL3-Asn-Npg-B5-Asp-3COOHF-Ala-Ala-Phe-Lys*3-PyrS2-2F3MeF-BztA-GlnR*3-Leu-Ser- |
| | NH2 |
| I-143 | Ac-PL3-Asn-Npg-B5-Asp-3COOHF-nLeu-Ala-Phe-Lys*3-PyrS2-2F3MeF-BztA-GlnR*3-Leu- |
| | Ser-NH2 |
| I-144 | Ac-PL3-Asn-Npg-B5-Asp-3COOHF-Aib-Ala-Phe-Lys*3-PyrS2-2F3MeF-BztA-GlnR*3-Leu-Ser- |
| | NH2 |
| I-145 | Ac-PL3-Hse-Npg-B5-Asp-3COOHF-Ala-Ala-Phe-Lys*3-PyrS2-2F3MeF-BztA-GlnR*3-Leu-Ser- |
| | NH2 |
| I-146 | Ac-PL3-Hse-Npg-B5-Asp-3COOHF-nLeu-Ala-Phe-Lys*3-PyrS2-2F3MeF-BztA-GlnR*3-Leu- |
| | Ser-NH2 |
| I-147 | Ac-PL3-Hse-Npg-B5-Asp-3COOHF-Aib-Ala-Phe-Lys*3-PyrS2-2F3MeF-BztA-GlnR*3-Leu-Ser- |
| | NH2 |
| I-148 | Ac-PL3-Asn-Cha-B5-Asp-Gln-Hse-Ala-Phe-GlnR*3-PyrS2-Phe-BztA-Lys*3-NH2 |
| I-149 | Ac-PL3-Asn-Cha-B5-Asp-Gln-iPrLys-Ala-Phe-GlnR*3-PyrS2-Phe-BztA-Lys*3-NH2 |
| I-150 | Ac-PL3-Asn-Cha-B5-Asp-Thr-iPrLys-Ala-Phe-GlnR*3-PyrS2-Phe-BztA-Lys*3-NH2 |
| I-151 | Ac-PL3-Asn-Cha-B5-Asp-Gln-Hse-Ala-Phe-GlnR*3-PyrS2-3Thi-BztA-Lys*3-NH2 |
| I-152 | Ac-PL3-Asn-Cha-B5-Asp-Gln-iPrLys-Ala-Phe-GlnR*3-PyrS2-3Thi-BztA-Lys*3-NH2 |
| I-153 | Ac-PL3-Asn-Cha-B5-Asp-Gln-Hse-Ala-Phe-GlnR*3-PyrS2-Phe-BztA-Lys*3-NH2 |
| I-154 | Ac-PL3-Asn-Cha-B5-Asp-Gln-iPrLys-Ala-Phe-GlnR*3-PyrS2-Phe-BztA-Lys*3-NH2 |
| I-155 | Ac-PL3-Asn-Cha-B5-Asp-Thr-Hse-Ala-Phe-GlnR*3-PyrS2-Phe-BztA-Lys*3-NH2 |
| I-156 | Ac-PL3-Asn-Cha-B5-Asp-Thr-iPrLys-Ala-Phe-GlnR*3-PyrS2-Phe-BztA-Lys*3-NH2 |
| I-157 | Ac-PL3-Asn-Cha-B5-Asp-Gln-Hse-Ala-Phe-GlnR*3-PyrS2-3Thi-BztA-Lys*3-NH2 |
| I-158 | Ac-PL3-Asn-Cha-B5-Asp-Gln-iPrLys-Ala-Phe-GlnR*3-PyrS2-3Thi-BztA-Lys*3-NH2 |
| I-159 | Ac-PL3-Asn-Npg-B5-Asp-3COOHF-Aib-Ala-Phe-Lys*3-PyrS2-3Thi-BztA-GlnR*3-Ala-NH2 |
| I-160 | Ac-PL3-Ser-Npg-B5-Asp-3COOHF-Aib-Ala-Phe-Lys*3-PyrS2-3Thi-BztA-GlnR*3-Ala-NH2 |
| I-161 | Ac-PL3-Thr-Npg-B5-Asp-3COOHF-Aib-Ala-Phe-Lys*3-PyrS2-3Thi-BztA-GlnR*3-Ala-NH2 |
| I-162 | Ac-PL3-Hse-Npg-B5-Asp-3COOHF-Aib-Ala-Phe-Lys*3-PyrS2-3Thi-BztA-GlnR*3-Ala-NH2 |
| I-163 | Ac-PL3-aThr-Npg-B5-Asp-3COOHF-Aib-Ala-Phe-Lys*3-PyrS2-3Thi-BztA-GlnR*3-Ala-NH2 |
| I-164 | Ac-PL3-MeAsn-Npg-B5-Asp-3COOHF-Aib-Ala-Phe-Lys*3-PyrS2-3Thi-BztA-GlnR*3-Ala-NH2 |
| I-165 | Ac-PL3-Asp-Npg-B5-Asn-3COOHF-Aib-Ala-Phe-Lys*3-PyrS2-3Thi-BztA-GlnR*3-Ala-NH2 |
| I-166 | Ac-PL3-Asp-Npg-B5-Ser-3COOHF-Aib-Ala-Phe-Lys*3-PyrS2-3Thi-BztA-GlnR*3-Ala-NH2 |
| I-167 | Ac-PL3-Asp-Npg-B5-Thr-3COOHF-Aib-Ala-Phe-Lys*3-PyrS2-3Thi-BztA-GlnR*3-Ala-NH2 |
| I-168 | Ac-PL3-Asp-Npg-B5-Hse-3COOHF-Aib-Ala-Phe-Lys*3-PyrS2-3Thi-BztA-GlnR*3-Ala-NH2 |
| I-169 | Ac-PL3-Asp-Npg-B5-aThr-3COOHF-Aib-Ala-Phe-Lys*3-PyrS2-3Thi-BztA-GlnR*3-Ala-NH2 |
| I-170 | Ac-PL3-Asp-Npg-B5-MeAsn-3COOHF-Aib-Ala-Phe-Lys*3-PyrS2-3Thi-BztA-GlnR*3-Ala-NH2 |
| I-171 | Ac-PL3-Asn-Npg-B5-Asp-3COOHF-Aib-Ala-Phe-Lys*3-PyrS2-3Thi-BztA-GlnR*3-Ala-NH2 |
| I-172 | Ac-PL3-Ser-Npg-B5-Asp-3COOHF-Aib-Ala-Phe-Lys*3-PyrS2-3Thi-BztA-GlnR*3-Ala-NH2 |
| I-173 | Ac-PL3-Thr-Npg-B5-Asp-3COOHF-Aib-Ala-Phe-Lys*3-PyrS2-3Thi-BztA-GlnR*3-Ala-NH2 |
| I-174 | Ac-PL3-Hse-Npg-B5-Asp-3COOHF-Aib-Ala-Phe-Lys*3-PyrS2-3Thi-BztA-GlnR*3-Ala-NH2 |
| I-175 | Ac-PL3-aThr-Npg-B5-Asp-3COOHF-Aib-Ala-Phe-Lys*3-PyrS2-3Thi-BztA-GlnR*3-Ala-NH2 |
| I-176 | Ac-PL3-MeAsn-Npg-B5-Asp-3COOHF-Aib-Ala-Phe-Lys*3-PyrS2-3Thi-BztA-GlnR*3-Ala-NH2 |
| I-177 | Ac-PL3-Asp-Npg-B5-Asn-3COOHF-Aib-Ala-Phe-Lys*3-PyrS2-3Thi-BztA-GlnR*3-Ala-NH2 |
| I-178 | Ac-PL3-Asp-Npg-B5-Ser-3COOHF-Aib-Ala-Phe-Lys*3-PyrS2-3Thi-BztA-GlnR*3-Ala-NH2 |
| I-179 | Ac-PL3-Asp-Npg-B5-Thr-3COOHF-Aib-Ala-Phe-Lys*3-PyrS2-3Thi-BztA-GlnR*3-Ala-NH2 |
| I-180 | Ac-PL3-Asp-Npg-B5-Hse-3COOHF-Aib-Ala-Phe-Lys*3-PyrS2-3Thi-BztA-GlnR*3-Ala-NH2 |
| I-181 | Ac-PL3-Asp-Npg-B5-aThr-3COOHF-Aib-Ala-Phe-Lys*3-PyrS2-3Thi-BztA-GlnR*3-Ala-NH2 |
| I-182 | Ac-PL3-Asp-Npg-B5-MeAsn-3COOHF-Aib-Ala-Phe-Lys*3-PyrS2-3Thi-BztA-GlnR*3-Ala-NH2 |
| I-183 | Ac-PL3-Asp-Npg-B5-Asp-3COOHF-Ala-Ala-Phe-Lys*3-PyrS2-3Thi-BztA-hGlnR*3-Ala-NH2 |
| I-184 | Ac-PL3-Asp-Npg-B5-Asp-3COOHF-Ala-Ala-Phe-Lys*3-PyrS2-2F3MeF-BztA-hGlnR*3-Ala- |
| | NH2 |
| I-185 | Ac-PL3-Asp-Npg-B5-Asp-3COOHF-Ala-Ala-Phe-hGlnR*3-PyrS2-3Thi-BztA-Lys*3-Ala-NH2 |
| I-186 | Ac-PL3-Asp-Npg-B5-Asp-3COOHF-Ala-Ala-Phe-hGlnR*3-PyrS2-2F3MeF-BztA-Lys*3-Ala- |
| | NH2 |
| I-187 | Ac-PL3-Asp-Npg-B5-Asp-3COOHF-Ala-Ala-Phe-iPrLys*3-PyrS2-3Thi-BztA-hGlnR*3-Ala- |
| | NH2 |
| I-188 | Ac-PL3-Asp-Npg-B5-Asp-3COOHF-Ala-Ala-Phe-iPrLys*3-PyrS2-2F3MeF-BztA-hGlnR*3-Ala- |
| | NH2 |
| I-189 | Ac-PL3-Asp-Npg-B5-Asp-3COOHF-Ala-Ala-Phe-hGlnR*3-PyrS2-2F3MeF-BztA-iPrLys*3-Ala- |
| | NH2 |
| I-190 | Ac-PL3-Asp-Npg-B5-Asp-3COOHF-Ala-Ala-Phe-iPrLys*3-PyrS2-3Thi-BztA-GlnR*3-Ala-NH2 |
| I-191 | Ac-PL3-Asp-Npg-B5-Asp-3COOHF-Ala-Ala-Phe-iPrLys*3-PyrS2-2F3MeF-BztA-GlnR*3-Ala- |
| | NH2 |
| I-192 | Ac-PL3-Asp-Npg-B5-Asp-3COOHF-Ala-Ala-Phe-GlnR*3-PyrS2-2F3MeF-BztA-iPrLys*3-Ala- |
| | NH2 |
| I-193 | Ac-PL3-Asp-Npg-B5-Asp-20H3COOHF-Aib-Ala-Phe-Lys*3-PyrS2-2F3MeF-BztA-GlnR*3-Ala- |
| | NH2 |
| I-194 | Ac-PL3-Asp-Npg-B5-Asp-40H3COOHF-Aib-Ala-Phe-Lys*3-PyrS2-2F3MeF-BztA-GlnR*3-Ala- |
| | NH2 |
| I-195 | Ac-PL3-Asp-Npg-B5-Asp-[CH2CMe2CO2H]TriAzDap-Aib-Ala-Phe-Lys*3-PyrS2-2F3MeF- |
| | BztA-GlnR*3-Ala-NH2 |
| I-196 | Ac-PL3-Asp-Npg-B5-Asp-4COOHF-Aib-Ala-Phe-Lys*3-PyrS2-2F3MeF-BztA-GlnR*3-Ala-NH2 |
| I-197 | Ac-PL3-Asp-Npg-B5-Asp-2COOHF-Aib-Ala-Phe-Lys*3-PyrS2-2F3MeF-BztA-GlnR*3-Ala-NH2 |
| I-198 | Ac-PL3-Asp-Npg-B5-Asp-Glu-Aib-Ala-Phe-Lys*3-PyrS2-2F3MeF-BztA-GlnR*3-Ala-NH2 |
| I-199 | Ac-PL3-Asp-Npg-B5-Asp-Asp-Aib-Ala-Phe-Lys*3-PyrS2-2F3MeF-BztA-GlnR*3-Ala-NH2 |
| I-200 | Ac-PL3-Asp-Npg-B5-Asp-Thr-Aib-Ala-Phe-Lys*3-PyrS2-2F3MeF-BztA-GlnR*3-Ala-NH2 |
| I-201 | Ac-PL3-Asp-Npg-B5-Asp-Gln-Aib-Ala-Phe-Lys*3-PyrS2-2F3MeF-BztA-GlnR*3-Ala-NH2 |
| I-202 | Ac-PL3-Asp-Npg-B5-Asp-His-Aib-Ala-Phe-Lys*3-PyrS2-2F3MeF-BztA-GlnR*3-Ala-NH2 |
| I-203 | Ac-PL3-Asp-Npg-B5-Asp-Tyr-Aib-Ala-Phe-Lys*3-PyrS2-2F3MeF-BztA-GlnR*3-Ala-NH2 |
| I-204 | Ac-PL3-Asp-Npg-B5-Asp-5F3Me2COOHF-Aib-Ala-Phe-Lys*3-PyrS2-2F3MeF-BztA-GlnR*3- |
| | Ala-NH2 |
| I-205 | Ac-PL3-Asp-Npg-B5-Asp-4F3Me2COOHF-Aib-Ala-Phe-Lys*3-PyrS2-2F3MeF-BztA-GlnR*3- |
| | Ala-NH2 |
| I-206 | Ac-PL3-Asp-Npg-B5-Asp-5F3Me3COOHF-Aib-Ala-Phe-Lys*3-PyrS2-2F3MeF-BztA-GlnR*3- |
| | Ala-NH2 |
| I-207 | Ac-PL3-Asp-Npg-B5-Asp-4F3Me3COOHF-Aib-Ala-Phe-Lys*3-PyrS2-2F3MeF-BztA-GlnR*3- |
| | Ala-NH2 |
| I-208 | Ac-PL3-Asp-Npg-B5-Asp-3F2COOHF-Aib-Ala-Phe-Lys*3-PyrS2-2F3MeF-BztA-GlnR*3-Ala- |
| | NH2 |
| I-209 | Ac-PL3-Asp-Npg-B5-Asp-Val-Aib-Ala-Phe-Lys*3-PyrS2-2F3MeF-BztA-GlnR*3-Ala-NH2 |
| I-210 | Ac-PL3-Asp-Npg-B5-Asp-Ser-Aib-Ala-Phe-Lys*3-PyrS2-2F3MeF-BztA-GlnR*3-Ala-NH2 |
| I-211 | Ac-PL3-Asp-Npg-B5-Asp-Trp-Aib-Ala-Phe-Lys*3-PyrS2-2F3MeF-BztA-GlnR*3-Ala-NH2 |
| I-212 | Ac-PL3-Asp-Npg-B5-Asp-Asn-Aib-Ala-Phe-Lys*3-PyrS2-2F3MeF-BztA-GlnR*3-Ala-NH2 |
| I-213 | Ac-PL3-Asp-Npg-B5-Asp-Ala-Aib-Ala-Phe-Lys*3-PyrS2-2F3MeF-BztA-GlnR*3-Ala-NH2 |
| I-214 | Ac-PL3-Asp-Npg-B5-Asp-Arg-Aib-Ala-Phe-Lys*3-PyrS2-2F3MeF-BztA-GlnR*3-Ala-NH2 |
| I-215 | Ac-PL3-Asp-Npg-B5-Asp-dGlu-Aib-Ala-Phe-Lys*3-PyrS2-2F3MeF-BztA-GlnR*3-Ala-NH2 |
| I-216 | Ac-PL3-Asp-Npg-B5-Asp-aThr-Aib-Ala-Phe-Lys*3-PyrS2-2F3MeF-BztA-GlnR*3-Ala-NH2 |
| I-217 | Ac-PL3-Asp-Npg-B5-Asp-hTyr-Aib-Ala-Phe-Lys*3-PyrS2-2F3MeF-BztA-GlnR*3-Ala-NH2 |
| I-218 | Ac-PL3-Asp-Npg-B5-Asp-3cbmf-Aib-Ala-Phe-Lys*3-PyrS2-2F3MeF-BztA-GlnR*3-Ala-NH2 |
| I-219 | Ac-PL3-Asp-Npg-B5-Asp-Leu-Aib-Ala-Phe-Lys*3-PyrS2-2F3MeF-BztA-GlnR*3-Ala-NH2 |
| I-220 | Ac-PL3-Asp-Npg-B5-Asp-Phe-Aib-Ala-Phe-Lys*3-PyrS2-2F3MeF-BztA-GlnR*3-Ala-NH2 |
| I-221 | Ac-PL3-Asp-Npg-B5-Asp-Lys-Aib-Ala-Phe-Lys*3-PyrS2-2F3MeF-BztA-GlnR*3-Ala-NH2 |
| I-222 | Ac-PL3-Asp-Npg-B5-Asp-Ile-Aib-Ala-Phe-Lys*3-PyrS2-2F3MeF-BztA-GlnR*3-Ala-NH2 |
| I-223 | Ac-PL3-Asp-Npg-B5-Asp-3COOHF-Aib-Ala-Phe-Lys*3-PyrS2-2F3MeF-BztA-GlnR*3-Ala- |
| | Serol |
| I-224 | Ac-PL3-Asp-Npg-B5-Asp-3COOHF-Aib-Ala-Phe-Lys*3-PyrS2-2F3MeF-BztA-GlnR*3- |
| | MorphNva-Serol |
| I-225 | Ac-PL3-Asp-Npg-B5-Asp-3COOHF-Aib-Ala-Phe-Lys*3-PyrS2-2F3MeF-BztA-GlnR*3- |
| | MorphNva-dAlaol |
| I-226 | Ac-PL3-Asp-Npg-B5-Asp-3COOHF-Aib-Ala-Phe-Lys*3-PyrS2-2F3MeF-BztA-GlnR*3-Ala- |
| | NHEt |
| I-227 | Ac-PL3-Asp-Npg-B5-Asp-3COOHF-Aib-Ala-Phe-Lys*3-PyrS2-2F3MeF-BztA-GlnR*3-Ala-Ser- |
| | NHEt |
| I-228 | Ac-PL3-Asp-Npg-B5-Asp-3COOHF-Aib-Ala-Phe-Lys*3-PyrS2-2F3MeF-BztA-GlnR*3- |
| | MorphNva-NHEt |
| I-229 | Ac-PL3-Asp-Npg-B5-Asp-3COOHF-Aib-Ala-Phe-Lys*3-PyrS2-2F3MeF-BztA-GlnR*3- |
| | MorphNva-Ser-NHEt |
| I-230 | Ac-PL3-Asp-Npg-B5-Asp-3COOHF-Aib-Ala-Phe-Lys*3-PyrS2-2F3MeF-BztA-GlnR*3-Ala-Ser- |
| | NH2 |
| I-231 | Ac-PL3-Asp-Npg-B5-Asp-3COOHF-Aib-Ala-Phe-Lys*3-PyrS2-2F3MeF-BztA-GlnR*3- |
| | MorphNva-NH2 |
| I-232 | Ac-PL3-Asp-Npg-B5-Asp-3COOHF-Aib-Ala-Phe-Lys*3-PyrS2-2F3MeF-BztA-GlnR*3- |
| | MorphNva-Ser-NH2 |
| I-233 | Ac-MePro-Asp-Npg-R4-Asp-3COOHF-Aib-Ala-Phe-Lys*3-PyrS2-3Thi-BztA-GlnR*3-Ala-NH2 |
| I-234 | Ac-MePro-Asp-Npg-R5-Asp-3COOHF-Aib-Ala-Phe-Lys*3-PyrS2-3Thi-BztA-GlnR*3-Ala-NH2 |
| I-235 | Ac-MePro-Asp-Npg-R6-Asp-3COOHF-Aib-Ala-Phe-Lys*3-PyrS2-3Thi-BztA-GlnR*3-Ala-NH2 |
| I-236 | Ac-MePro-Asp-Npg-R5-Asp-3COOHF-Ala-Ala-Phe-Lys*3-PyrS2-3Thi-BztA-GlnR*3-Ala-NH2 |
| I-237 | Ac-MePro-Asp-Val-R5-Asp-3COOHF-Aib-Ala-Phe-Lys*3-PyrS2-3Thi-BztA-GlnR*3-Ala-NH2 |
| I-238 | Ac-MePro-Asp-nLeu-R5-Asp-3COOHF-Aib-Ala-Phe-Lys*3-PyrS2-3Thi-BztA-GlnR*3-Ala-NH2 |
| I-239 | Ac-MePro-Asp-Npg-R4-Asp-TfeGA-Aib-Ala-Phe-Lys*3-PyrS2-3Thi-BztA-GlnR*3-Ala-NH2 |
| I-240 | Ac-MePro-Asp-Npg-R5-Asp-TfeGA-Aib-Ala-Phe-Lys*3-PyrS2-3Thi-BztA-GlnR*3-Ala-NH2 |
| I-241 | Ac-MePro-Asp-Npg-R6-Asp-TfeGA-Aib-Ala-Phe-Lys*3-PyrS2-3Thi-BztA-GlnR*3-Ala-NH2 |
| I-242 | Ac-MePro-Asp-Npg-R5-Asp-TfeGA-Ala-Ala-Phe-Lys*3-PyrS2-3Thi-BztA-GlnR*3-Ala-NH2 |
| I-243 | Ac-MePro-Asp-Val-R5-Asp-TfeGA-Aib-Ala-Phe-Lys*3-PyrS2-3Thi-BztA-GlnR*3-Ala-NH2 |
| I-244 | Ac-MePro-Asp-nLeu-R5-Asp-TfeGA-Aib-Ala-Phe-Lys*3-PyrS2-3Thi-BztA-GlnR*3-Ala-NH2 |
| I-245 | Ac-PL3-Asp-nLeu-B5-Asp-3COOHF-Aib-Ala-Phe-Lys*3-PyrS2-3Thi-BztA-GlnR*3-Ala-NH2 |
| I-246 | Ac-PL3-Asp-Leu-B5-Asp-3COOHF-Aib-Ala-Phe-Lys*3-PyrS2-3Thi-BztA-GlnR*3-Ala-NH2 |
| I-247 | Ac-PL3-Asp-Ile-B5-Asp-3COOHF-Aib-Ala-Phe-Lys*3-PyrS2-3Thi-BztA-GlnR*3-Ala-NH2 |
| I-248 | Ac-PL3-Asp-Val-B5-Asp-3COOHF-Aib-Ala-Phe-Lys*3-PyrS2-3Thi-BztA-GlnR*3-Ala-NH2 |
| I-249 | Ac-PL3-Asp-Phe-B5-Asp-3COOHF-Aib-Ala-Phe-Lys*3-PyrS2-3Thi-BztA-GlnR*3-Ala-NH2 |
| I-250 | Ac-PL3-Asp-Cha-B5-Asp-3COOHF-Aib-Ala-Phe-Lys*3-PyrS2-3Thi-BztA-GlnR*3-Ala-NH2 |
| I-251 | Ac-PL3-Asp-CypA-B5-Asp-3COOHF-Aib-Ala-Phe-Lys*3-PyrS2-3Thi-BztA-GlnR*3-Ala-NH2 |
| I-252 | Ac-PL3-Asp-CyLeu-B5-Asp-3COOHF-Aib-Ala-Phe-Lys*3-PyrS2-3Thi-BztA-GlnR*3-Ala-NH2 |
| I-253 | Ac-PL3-Asp-Chg-B5-Asp-3COOHF-Aib-Ala-Phe-Lys*3-PyrS2-3Thi-BztA-GlnR*3-Ala-NH2 |
| I-254 | Ac-PL3-Asp-Pff-B5-Asp-3COOHF-Aib-Ala-Phe-Lys*3-PyrS2-3Thi-BztA-GlnR*3-Ala-NH2 |
| I-255 | Ac-PL3-Asp-DiethA-B5-Asp-3COOHF-Aib-Ala-Phe-Lys*3-PyrS2-3Thi-BztA-GlnR*3-Ala-NH2 |
| I-256 | Ac-PL3-Asp-Npg-B5-Asp-3COOHF-Ala-Ala-Phe-4PipA*3-PyrS2-3Thi-BztA-GlnR*3-Ala-NH2 |
| I-257 | Ac-PL3-Asp-Npg-B5-Asp-3COOHF-Ala-Ala-Phe-4PipA*3-PyrS2-2F3MeF-BztA-GlnR*3-Ala- |
| | NH2 |
| I-258 | Ac-PL3-Asp-Npg-B5-Asp-3COOHF-Ala-Ala-Phe-GlnR*3-PyrS2-3Thi-BztA-4PipA*3-Ala-NH2 |
| I-259 | Ac-PL3-Asp-Npg-B5-Asp-3COOHF-Ala-Ala-Phe-GlnR*3-PyrS2-2F3MeF-BztA-4PipA*3-Ala- |
| | NH2 |
| I-260 | Ac-PL3-Asp-Npg-B5-Asp-3COOHF-Ala-Ala-Phe-hGlnR*3-PyrS2-3Thi-BztA-4PipA*3-Ala-NH2 |
| I-261 | Ac-PL3-Asp-Npg-B5-Asp-3COOHF-Ala-Ala-Phe-hGlnR*3-PyrS2-2F3MeF-BztA-4PipA*3-Ala- |
| | NH2 |
| I-262 | Ac-PL3-Asp-Npg-B5-Asp-3COOHF-Aib-Ala-Phe-TriAzLys*3-PyrS2-3Thi-BztA-sCH2S*3-Ala- |
| | NH2 |
| I-263 | Ac-PL3-Asp-Npg-B5-Asp-3COOHF-Aib-Ala-Phe-sCH2S*3-PyrS2-3Thi-BztA-TriAzLys*3-Ala- |
| | NH2 |
| I-264 | Ac-PL3-Asp-Npg-B5-Asp-3COOHF-Ala-Ala-Phe-TriAzLys*3-PyrS2-3Thi-BztA-sCH2S*3-Ala- |
| | NH2 |
| I-265 | Ac-PL3-Asp-Npg-B5-Asp-3COOHF-Ala-Ala-Phe-sCH2S*3-PyrS2-3Thi-BztA-TriAzLys*3-Ala- |
| | NH2 |
| I-266 | Ac-PL3-Asp-Npg-B5-Asp-3COOHF-Aib-Ala-Phe-Lys*3-PyrS2-Ala-BztA-GlnR*3-Ala-NH2 |
| I-267 | Ac-PL3-Asp-Npg-B5-Asp-3COOHF-Aib-Ala-Phe-Lys*3-PyrS2-Ala-BztA-GlnR*3-Ala-NH2 |
| I-268 | Ac-PL3-Asp-Npg-B5-Asp-3COOHF-Aib-Ala-Phe-Lys*3-PyrS2-Abu-BztA-GlnR*3-Ala-NH2 |
| I-269 | Ac-PL3-Asp-Npg-B5-Asp-3COOHF-Aib-Ala-Phe-Lys*3-PyrS2-Abu-BztA-GlnR*3-Ala-NH2 |
| I-270 | Ac-PL3-Asp-Npg-B5-Asp-3COOHF-Aib-Ala-Phe-Lys*3-PyrS2-Nva-BztA-GlnR*3-Ala-NH2 |
| I-271 | Ac-PL3-Asp-Npg-B5-Asp-3COOHF-Aib-Ala-Phe-Lys*3-PyrS2-nLeu-BztA-GlnR*3-Ala-NH2 |
| I-272 | Ac-PL3-Asp-Npg-B5-Asp-3COOHF-Aib-Ala-Phe-Lys*3-PyrS2-nLeu-BztA-GlnR*3-Ala-NH2 |
| I-273 | Ac-PL3-Asp-Npg-B5-Asp-3COOHF-Aib-Ala-Phe-Lys*3-PyrS2-Leu-BztA-GlnR*3-Ala-NH2 |
| I-274 | Ac-PL3-Asp-Npg-B5-Asp-3COOHF-Aib-Ala-Phe-Lys*3-PyrS2-Leu-BztA-GlnR*3-Ala-NH2 |
| I-275 | Ac-PL3-Asp-Npg-B5-Asp-3COOHF-Aib-Ala-Phe-Lys*3-PyrS2-hLeu-BztA-GlnR*3-Ala-NH2 |
| I-276 | Ac-PL3-Asp-Npg-B5-Asp-3COOHF-Aib-Ala-Phe-Lys*3-PyrS2-hLeu-BztA-GlnR*3-Ala-NH2 |
| I-277 | Ac-PL3-Asp-Npg-B5-Asp-3COOHF-Aib-Ala-Phe-Lys*3-PyrS2-Npg-BztA-GlnR*3-Ala-NH2 |
| I-278 | Ac-PL3-Asp-Npg-B5-Asp-3COOHF-Aib-Ala-Phe-Lys*3-PyrS2-Npg-BztA-GlnR*3-Ala-NH2 |
| I-279 | Ac-PL3-Asp-Npg-B5-Asp-3COOHF-Aib-Ala-Phe-Lys*3-PyrS2-Phe-BztA-GlnR*3-Ala-NH2 |
| I-280 | Ac-PL3-Asp-Npg-B5-Asp-3COOHF-Aib-Ala-Phe-Lys*3-PyrS2-Phe-BztA-GlnR*3-Ala-NH2 |
| I-281 | Ac-PL3-Asp-Npg-B5-Asp-3COOHF-Aib-Ala-Phe-Lys*3-PyrS2-Cpa-BztA-GlnR*3-Ala-NH2 |
| I-282 | Ac-PL3-Asp-Npg-B5-Asp-3COOHF-Aib-Ala-Phe-Lys*3-PyrS2-Cpa-BztA-GlnR*3-Ala-NH2 |
| I-283 | Ac-PL3-Asp-Npg-B5-Asp-3COOHF-Aib-Ala-Phe-Lys*3-PyrS2-Cba-BztA-GlnR*3-Ala-NH2 |
| I-284 | Ac-PL3-Asp-Npg-B5-Asp-3COOHF-Aib-Ala-Phe-Lys*3-PyrS2-CypA-BztA-GlnR*3-Ala-NH2 |
| I-285 | Ac-PL3-Asp-Npg-B5-Asp-3COOHF-Aib-Ala-Phe-Lys*3-PyrS2-ChA-BztA-GlnR*3-Ala-NH2 |
| I-286 | Ac-PL3-Asp-Npg-B5-SbMeAsp-3COOHF-Aib-Ala-Phe-Lys*3-PyrS2-2F3MeF-BztA-GlnR*3- |
| | Ala-NH2 |
| I-287 | Ac-PL3-Asp-Npg-B5-RbMeAsp-3COOHF-Aib-Ala-Phe-Lys*3-PyrS2-2F3MeF-BztA-GlnR*3- |
| | Ala-NH2 |
| I-288 | Ac-PL3-SbMeAsp-Npg-B5-Asp-3COOHF-Aib-Ala-Phe-Lys*3-PyrS2-2F3MeF-BztA-GlnR*3- |
| | Ala-NH2 |
| I-289 | Ac-PL3-RbMeAsp-Npg-B5-Asp-3COOHF-Aib-Ala-Phe-Lys*3-PyrS2-2F3MeF-BztA-GlnR*3- |
| | Ala-NH2 |
| I-290 | Ac-PL3-aMeDAsp-Npg-B5-Asp-3COOHF-Aib-Ala-Phe-Lys*3-PyrS2-2F3MeF-BztA-GlnR*3- |
| | Ala-NH2 |
| I-291 | Ac-PL3-Glu-Npg-B5-Glu-3COOHF-Aib-Ala-Phe-Lys*3-PyrS2-2F3MeF-BztA-GlnR*3-Ala-NH2 |
| I-292 | Ac-PL3-Asp-Npg-B5-Asp-3COOHF-Aib-Ala-Phe-Lys*3-PyrS2-2FurA-BztA-GlnR*3-Ala-NH2 |
| I-293 | Ac-PL3-Asp-Npg-B5-Asp-3COOHF-Aib-Ala-Phe-Lys*3-PyrS2-20MeF-BztA-GlnR*3-Ala-NH2 |
| I-294 | Ac-PL3-Asp-Npg-B5-Asp-3COOHF-Aib-Ala-Phe-Lys*3-PyrS2-2MeF-BztA-GlnR*3-Ala-NH2 |
| I-295 | Ac-PL3-Asp-Npg-B5-Asp-3COOHF-Aib-Ala-Phe-Lys*3-PyrS2-2BrF-BztA-GlnR*3-Ala-NH2 |
| I-296 | Ac-PL3-Asp-Npg-B5-Asp-3COOHF-Aib-Ala-Phe-Lys*3-PyrS2-2ClF-BztA-GlnR*3-Ala-NH2 |
| I-297 | Ac-PL3-Asp-Npg-B5-Asp-3COOHF-Aib-Ala-Phe-Lys*3-PyrS2-2CNF-BztA-GlnR*3-Ala-NH2 |
| I-298 | Ac-PL3-Asp-Npg-B5-Asp-3COOHF-Aib-Ala-Phe-Lys*3-PyrS2-2NO2F-BztA-GlnR*3-Ala-NH2 |
| I-299 | Ac-PL3-Asp-Npg-B5-Asp-3COOHF-Aib-Ala-Phe-Lys*3-PyrS2-2PyrA-BztA-GlnR*3-Ala-NH2 |
| I-300 | Ac-PL3-Asp-Npg-B5-Asp-3COOHF-Aib-Ala-Phe-Lys*3-PyrS2-3PyrA-BztA-GlnR*3-Ala-NH2 |
| I-301 | Ac-PL3-Asp-Npg-B5-Asp-3COOHF-Aib-Ala-Phe-Lys*3-PyrS2-4PyrA-BztA-GlnR*3-Ala-NH2 |
| I-302 | Ac-PL3-Asp-Npg-B5-Asp-3COOHF-Aib-Ala-Phe-Lys*3-PyrS2-His-BztA-GlnR*3-Ala-NH2 |
| I-303 | Ac-PL3-Asp-Npg-B5-Asp-3COOHF-Aib-Ala-Phe-Lys*3-PyrS2-2F3MeF-BztA-GlnR*3- |
| | [BiotinPEG8]Lys-NH2 |
| I-304 | Ac-PL3-Asp-Ala-B5-Asp-3COOHF-Aib-Ala-Phe-Lys*3-PyrS2-3Thi-BztA-GlnR*3-Ala-NH2 |
| I-305 | Ac-PL3-Asp-Tyr-B5-Asp-3COOHF-Aib-Ala-Phe-Lys*3-PyrS2-3Thi-BztA-GlnR*3-Ala-NH2 |
| I-306 | Ac-PL3-Asp-Trp-B5-Asp-3COOHF-Aib-Ala-Phe-Lys*3-PyrS2-3Thi-BztA-GlnR*3-Ala-NH2 |
| I-307 | Ac-PL3-Asp-Ser-B5-Asp-3COOHF-Aib-Ala-Phe-Lys*3-PyrS2-3Thi-BztA-GlnR*3-Ala-NH2 |
| I-308 | Ac-PL3-Asp-Aib-B5-Asp-3COOHF-Aib-Ala-Phe-Lys*3-PyrS2-3Thi-BztA-GlnR*3-Ala-NH2 |
| I-309 | Ac-PL3-Asp-Phg-B5-Asp-3COOHF-Aib-Ala-Phe-Lys*3-PyrS2-3Thi-BztA-GlnR*3-Ala-NH2 |
| I-310 | Ac-PL3-Asp-DipA-B5-Asp-3COOHF-Aib-Ala-Phe-Lys*3-PyrS2-3Thi-BztA-GlnR*3-Ala-NH2 |
| I-311 | Ac-PL3-Asp-OctG-B5-Asp-3COOHF-Aib-Ala-Phe-Lys*3-PyrS2-3Thi-BztA-GlnR*3-Ala-NH2 |
| I-312 | Ac-PL3-Asp-Cba-B5-Asp-3COOHF-Aib-Ala-Phe-Lys*3-PyrS2-3Thi-BztA-GlnR*3-Ala-NH2 |
| I-313 | Ac-PL3-Asp-MorphNva-B5-Asp-3COOHF-Aib-Ala-Phe-Lys*3-PyrS2-3Thi-BztA-GlnR*3-Ala- |
| | NH2 |
| I-314 | Ac-PL3-Asp-F2PipNva-B5-Asp-3COOHF-Aib-Ala-Phe-Lys*3-PyrS2-3Thi-BztA-GlnR*3-Ala- |
| | NH2 |
| I-315 | Ac-PL3-Asn-Npg-B5-Asp-[CH2CMe2CO2H]TriAzDap-Aib-Ala-Phe-Lys*3-PyrS2-3Thi-BztA- |
| | GlnR*3-Ala-NH2 |
| I-316 | Ac-PL3-Ser-Npg-B5-Asp-[CH2CMe2CO2H]TriAzDap-Aib-Ala-Phe-Lys*3-PyrS2-3Thi-BztA- |
| | GlnR*3-Ala-NH2 |
| I-317 | Ac-PL3-aThr-Npg-B5-Asp-[CH2CMe2CO2H]TriAzDap-Aib-Ala-Phe-Lys*3-PyrS2-3Thi-BztA- |
| | GlnR*3-Ala-NH2 |
| I-318 | Ac-PL3-Asp-Npg-B5-Asn-[CH2CMe2CO2H]TriAzDap-Aib-Ala-Phe-Lys*3-PyrS2-3Thi-BztA- |
| | GlnR*3-Ala-NH2 |
| I-319 | Ac-PL3-Asn-Npg-B5-Asp-[CH2CMe2CO2H]TriAzDap-MorphGln-Ala-Phe-Lys*3-PyrS2-3Thi- |
| | BztA-GlnR*3-Ala-NH2 |
| I-320 | Ac-PL3-Ser-Npg-B5-Asp-[CH2CMe2CO2H]TriAzDap-MorphGln-Ala-Phe-Lys*3-PyrS2-3Thi- |
| | BztA-GlnR*3-Ala-NH2 |
| I-321 | Ac-PL3-aThr-Npg-B5-Asp-[CH2CMe2CO2H]TriAzDap-MorphGln-Ala-Phe-Lys*3-PyrS2-3Thi- |
| | BztA-GlnR*3-Ala-NH2 |
| I-322 | Ac-PL3-Asp-Npg-B5-Asn-[CH2CMe2CO2H]TriAzDap-MorphGln-Ala-Phe-Lys*3-PyrS2-3Thi- |
| | BztA-GlnR*3-Ala-NH2 |
| I-323 | Ac-PL3-Asn-Npg-B5-Asp-3COOHF-MorphGln-Ala-Phe-Lys*3-PyrS2-3Thi-BztA-GlnR*3-Ala- |
| | NH2 |
| I-324 | Ac-PL3-Ser-Npg-B5-Asp-3COOHF-MorphGln-Ala-Phe-Lys*3-PyrS2-3Thi-BztA-GlnR*3-Ala- |
| | NH2 |
| I-325 | Ac-PL3-aThr-Npg-B5-Asp-3COOHF-MorphGln-Ala-Phe-Lys*3-PyrS2-3Thi-BztA-GlnR*3-Ala- |
| | NH2 |
| I-326 | Ac-PL3-Asp-Npg-B5-Asn-3COOHF-MorphGln-Ala-Phe-Lys*3-PyrS2-3Thi-BztA-GlnR*3-Ala- |
| | NH2 |
| I-327 | Ac-PL3-Asp-Npg-B5-Asp-3COOHF-Aib-Ala-Phe-Lys*3-PyrS2-2F3MeF-34ClF-GlnR*3-Ala- |
| | NH2 |
| I-328 | Ac-PL3-Asp-Npg-B5-Asp-3COOHF-Aib-Ala-Phe-Lys*3-PyrS2-3Thi-34ClF-GlnR*3-Ala-NH2 |
| I-329 | Ac-PL3-Asp-Npg-B5-Asp-3COOHF-Aib-Ala-Phe-Lys*3-PyrS2-Phe-34ClF-GlnR*3-Ala-NH2 |
| I-330 | Ac-PL3-Asp-Npg-B5-Asp-3COOHF-Aib-Ala-Phe-Lys*3-PyrS2-2F3MeF-34MeF-GlnR*3-Ala- |
| | NH2 |
| I-331 | Ac-PL3-Asp-Npg-B5-Asp-3COOHF-Aib-Ala-Phe-Lys*3-PyrS2-2F3MeF-3BrF-GlnR*3-Ala-NH2 |
| I-332 | Ac-PL3-Asp-Npg-B5-Asp-3COOHF-Aib-Ala-Phe-Lys*3-PyrS2-3Thi-3BrF-GlnR*3-Ala-NH2 |
| I-333 | Ac-PL3-Asp-Npg-B5-Asp-3COOHF-Aib-Ala-Phe-Lys*3-PyrS2-Phe-3BrF-GlnR*3-Ala-NH2 |
| I-334 | Ac-PL3-Asp-Npg-B5-Asp-3COOHF-Aib-Ala-Phe-Lys*3-PyrS2-1NapA-BztA-GlnR*3-Ala-NH2 |
| I-335 | Ac-PL3-Asp-Npg-B5-Asp-3COOHF-Aib-Ala-Phe-TriAzLys*3-PyrS2-3Thi-BztA-sAla*3-Ala- |
| | NH2 |
| I-336 | Ac-PL3-Asp-Npg-B5-Asp-3COOHF-Aib-Ala-Phe-sAla*3-PyrS2-3Thi-BztA-TriAzLys*3-Ala- |
| | NH2 |
| I-337 | Ac-PL3-Asp-Npg-B5-Asp-3COOHF-Ala-Ala-Phe-TriAzLys*3-PyrS2-3Thi-BztA-sAla*3-Ala- |
| | NH2 |
| I-338 | Ac-PL3-Asp-Npg-B5-Asp-3COOHF-Ala-Ala-Phe-sAla*3-PyrS2-3Thi-BztA-TriAzLys*3-Ala- |
| | NH2 |
| I-339 | Ac-PL3-Asp-Npg-B5-Asp-3COOHF-Aib-Ala-Phe-[4FB]Cys-PyrS2-2F3MeF-BztA-Cys-Ala-NH2 |
| I-340 | Ac-PL3-Asp-Npg-B5-Asp-3COOHF-Aib-Ala-Phe-[8FBB]Cys-PyrS2-2F3MeF-BztA-Cys-Ala- |
| | NH2 |
| I-341 | Ac-PL3-Asp-Npg-B5-Asp-3COOHF-Aib-Ala-Phe-[8FBB]Cys-PyrS2-2F3MeF-BztA-Cys-Ala- |
| | NH2 |
| I-342 | Ac-PL3-Asp-Npg-B5-Asp-3COOHF-Aib-Ala-Phe-Lys*3-PyrS2-3Thi-BztA-GlnR*3-Glu-Ala- |
| | NH2 |
| I-343 | Ac-PL3-Asp-Npg-B5-Asp-3COOHF-Aib-Ala-Phe-Lys*3-PyrS2-3Thi-BztA-GlnR*3-Ala-Glu- |
| | Ala-NH2 |
| I-344 | Ac-PL3-Asp-Npg-B5-Asp-3COOHF-Aib-Ala-Phe-Lys*3-PyrS2-2COOHF-BztA-GlnR*3-Ala- |
| | NH2 |
| I-345 | Ac-PL3-Asp-Npg-B5-Asp-3COOHF-Aib-Ala-3COOHF-Lys*3-PyrS2-3Thi-BztA-GlnR*3-Ala- |
| | NH2 |
| I-346 | Ac-PL3-Asp-Npg-B5-Asp-3COOHF-Aib-Ala-3COOHF-Lys*3-PyrS2-2COOHF-BztA-GlnR*3- |
| | Ala-NH2 |
| I-347 | Ac-PL3-Asp-Npg-B5-Asp-3COOHF-Aib-Ala-3COOHF-Lys*3-PyrS2-2COOHF-BztA-GlnR*3- |
| | Ala-Glu-Ala-NH2 |
| I-348 | Ac-PL3-Asp-Npg-B5-Asp-3COOHF-Aib-Ala-Phe-Lys*3-PyrS2-3Thi-BztA-GlnR*3-Glu-Ala-OH |
| I-349 | Ac-PL3-Asp-Npg-B5-Asp-3COOHF-Aib-Ala-Phe-Lys*3-PyrS2-2COOHF-BztA-GlnR*3-Ala-OH |
| I-350 | Ac-PL3-Asp-Npg-B5-Asp-3COOHF-Aib-Ala-3COOHF-Lys*3-PyrS2-3Thi-BztA-GlnR*3-Ala- |
| | OH |
| I-351 | Ac-PL3-Asp-Npg-B5-Asp-3COOHF-Aib-Ala-3COOHF-Lys*3-PyrS2-2COOHF-BztA-GlnR*3- |
| | Ala-OH |
| I-352 | Ac-PL3-Asp-Npg-B5-Asp-3COOHF-Aib-Ala-3COOHF-Lys*3-PyrS2-2COOHF-BztA-GlnR*3- |
| | Ala-Glu-Ala-OH |
| I-353 | Ac-PL3-Asp-Npg-B5-Asp-3COOHF-Aib-Ala-Cba-Lys*3-PyrS2-3Thi-BztA-GlnR*3-Ala-NH2 |
| I-354 | Ac-PL3-Asp-Npg-B5-Asp-3COOHF-Aib-Ala-CypA-Lys*3-PyrS2-3Thi-BztA-GlnR*3-Ala-NH2 |
| I-355 | Ac-PL3-Asp-Npg-B5-Asp-3COOHF-Aib-Ala-BztA-Lys*3-PyrS2-3Thi-BztA-GlnR*3-Ala-NH2 |
| I-356 | Ac-PL3-Asp-Npg-B5-Asp-3COOHF-Aib-Ala-1NapA-Lys*3-PyrS2-3Thi-BztA-GlnR*3-Ala-NH2 |
| I-357 | Ac-PL3-Asp-Npg-B5-Asp-3COOHF-Aib-Ala-2NapA-Lys*3-PyrS2-3Thi-BztA-GlnR*3-Ala-NH2 |
| I-358 | Ac-PL3-Asp-Npg-B5-Asp-3COOHF-Aib-Ala-Tyr-Lys*3-PyrS2-3Thi-BztA-GlnR*3-Ala-NH2 |
| I-359 | Ac-PL3-Asp-Npg-B5-Asp-3COOHF-Aib-Ala-Trp-Lys*3-PyrS2-3Thi-BztA-GlnR*3-Ala-NH2 |
| I-360 | Ac-PL3-Asp-Npg-B5-Asp-3COOHF-Aib-Ala-Leu-Lys*3-PyrS2-3Thi-BztA-GlnR*3-Ala-NH2 |
| I-361 | Ac-PL3-Asp-Npg-B5-Asp-3COOHF-Aib-Ala-Ile-Lys*3-PyrS2-3Thi-BztA-GlnR*3-Ala-NH2 |
| I-362 | Ac-PL3-Asp-Npg-B5-Asp-3COOHF-Aib-Ala-nLeu-Lys*3-PyrS2-3Thi-BztA-GlnR*3-Ala-NH2 |
| I-363 | Ac-PL3-Asp-Npg-B5-Asp-3COOHF-Aib-Ala-Ser-Lys*3-PyrS2-3Thi-BztA-GlnR*3-Ala-NH2 |
| I-364 | Ac-PL3-Asp-Npg-B5-Asp-3COOHF-Aib-Ala-3Thi-Lys*3-PyrS2-3Thi-BztA-GlnR*3-Ala-NH2 |
| I-365 | Ac-PL3-Asp-Npg-B5-Asp-3COOHF-Aib-Ala-2Thi-Lys*3-PyrS2-3Thi-BztA-GlnR*3-Ala-NH2 |
| I-366 | Ac-PL3-Asp-Npg-B5-Asp-3COOHF-Aib-Ala-Chg-Lys*3-PyrS2-3Thi-BztA-GlnR*3-Ala-NH2 |
| I-367 | Ac-PL3-Asp-Npg-B5-Asp-3COOHF-Aib-Ala-Hse-Lys*3-PyrS2-3Thi-BztA-GlnR*3-Ala-NH2 |
| I-368 | Ac-PL3-Asp-Npg-B5-Asp-3COOHF-Aib-Ala-4TriA-Lys*3-PyrS2-3Thi-BztA-GlnR*3-Ala-NH2 |
| I-369 | Ac-PL3-Asp-Npg-B5-Asp-3COOHF-Aib-Ala-3F3MeF-Lys*3-PyrS2-3Thi-BztA-GlnR*3-Ala- |
| | NH2 |
| I-370 | Ac-PL3-Asp-Npg-B5-Asp-3COOHF-Aib-Ala-Thr-Lys*3-PyrS2-3Thi-BztA-GlnR*3-Ala-NH2 |
| I-371 | Ac-PL3-Asp-Npg-B5-Asp-3COOHF-Aib-Ala-His-Lys*3-PyrS2-3Thi-BztA-GlnR*3-Ala-NH2 |
| I-372 | Ac-PL3-Asp-Npg-B5-Asp-3COOHF-Aib-Ala-Val-Lys*3-PyrS2-3Thi-BztA-GlnR*3-Ala-NH2 |
| I-373 | Ac-PL3-Asp-Npg-B5-Asp-3COOHF-Aib-Ala-Asn-Lys*3-PyrS2-3Thi-BztA-GlnR*3-Ala-NH2 |
| I-374 | Ac-PL3-Asp-Npg-B5-Asp-3COOHF-Aib-Ala-Gln-Lys*3-PyrS2-3Thi-BztA-GlnR*3-Ala-NH2 |
| I-375 | Ac-PL3-Asp-Npg-B5-Asp-3COOHF-Aib-Ala-Phe-[mXyl]Cys-PyrS2-2F3MeF-BztA-Cys-Ala- |
| | NH2 |
| I-376 | Ac-PL3-Asp-Npg-B5-Asp-3COOHF-Aib-Ala-Phe-[oXyl]Cys-PyrS2-2F3MeF-BztA-Cys-Ala- |
| | NH2 |
| I-377 | Ac-PL3-Asp-Npg-B5-Asp-3COOHF-Aib-Ala-Phe-[pXyl]Cys-PyrS2-2F3MeF-BztA-Cys-Ala- |
| | NH2 |
| I-378 | Ac-PL3-Asp-Npg-B5-Asp-3COOHF-MorphGln-Ala-Phe-Lys*3-PyrS2-3Thi-BztA-GlnR*3-Ala- |
| | NH2 |
| I-379 | Ac-PL3-Asp-Npg-B5-Asp-3COOHF-Me2Gln-Ala-Phe-Lys*3-PyrS2-3Thi-BztA-GlnR*3-Ala- |
| | NH2 |
| I-380 | Ac-PL3-Asp-Npg-B5-Asp-3COOHF-Met20-Ala-Phe-Lys*3-PyrS2-3Thi-BztA-GlnR*3-Ala-NH2 |
| I-381 | Ac-PL3-Asp-Npg-B5-Asp-3COOHF-AcLys-Ala-Phe-Lys*3-PyrS2-3Thi-BztA-GlnR*3-Ala-NH2 |
| I-382 | Ac-PL3-Asp-Npg-B5-Asp-3COOHF-CyLeu-Ala-Phe-Lys*3-PyrS2-3Thi-BztA-GlnR*3-Ala-NH2 |
| I-383 | Ac-PL3-Asp-Npg-B5-Asp-3COOHF-CyLeu-Ala-Phe-Lys*3-PyrS2-3Thi-BztA-GlnR*3-Ala-NH2 |
| I-384 | Ac-PL3-Asp-Npg-B5-Asp-3COOHF-Acp-Ala-Phe-Lys*3-PyrS2-3Thi-BztA-GlnR*3-Ala-NH2 |
| I-385 | Ac-PL3-Asp-Npg-B5-Asp-3COOHF-Acp-Ala-Phe-Lys*3-PyrS2-3Thi-BztA-GlnR*3-Ala-NH2 |
| I-386 | Ac-PL3-Asp-Npg-B5-Asp-3COOHF-Phe-Ala-Phe-Lys*3-PyrS2-3Thi-BztA-GlnR*3-Ala-NH2 |
| I-387 | Ac-PL3-Asp-Npg-B5-Asp-3COOHF-His-Ala-Phe-Lys*3-PyrS2-3Thi-BztA-GlnR*3-Ala-NH2 |
| I-388 | Ac-PL3-Asp-Npg-B5-Asp-3COOHF-Val-Ala-Phe-Lys*3-PyrS2-3Thi-BztA-GlnR*3-Ala-NH2 |
| I-389 | Ac-PL3-Asp-Npg-B5-Asp-3COOHF-Ser-Ala-Phe-Lys*3-PyrS2-3Thi-BztA-GlnR*3-Ala-NH2 |
| I-390 | Ac-PL3-Asp-Npg-B5-Asp-3COOHF-Ser-Ala-Phe-Lys*3-PyrS2-3Thi-BztA-GlnR*3-Ala-NH2 |
| I-391 | Ac-PL3-Asp-Npg-B5-Asp-3COOHF-Gln-Ala-Phe-Lys*3-PyrS2-3Thi-BztA-GlnR*3-Ala-NH2 |
| I-392 | Ac-PL3-Asp-Npg-B5-Asp-3COOHF-Gln-Ala-Phe-Lys*3-PyrS2-3Thi-BztA-GlnR*3-Ala-NH2 |
| I-393 | Ac-PL3-Asp-Npg-B5-Asp-3COOHF-Asn-Ala-Phe-Lys*3-PyrS2-3Thi-BztA-GlnR*3-Ala-NH2 |
| I-394 | Ac-PL3-Asp-Npg-B5-Asp-3COOHF-Asn-Ala-Phe-Lys*3-PyrS2-3Thi-BztA-GlnR*3-Ala-NH2 |
| I-395 | Ac-PL3-Asp-Npg-B5-Asp-3COOHF-Aib-Ala-2Cpg-Lys*3-PyrS2-3Thi-BztA-GlnR*3-Ala-NH2 |
| I-396 | Ac-PL3-Asp-Npg-B5-Asp-3COOHF-Aib-Ala-Phe-Lys*3-PyrS2-3Thi-BztA-GlnR*3-Val-NH2 |
| I-397 | Ac-PL3-Asp-Npg-B5-Asp-3COOHF-Aib-Ala-Phe-Lys*3-PyrS2-3Thi-BztA-GlnR*3-Aib-NH2 |
| I-398 | Ac-PL3-Asp-Npg-B5-Asp-3COOHF-Aib-Ala-Phe-Lys*3-PyrS2-3Thi-BztA-GlnR*3-Thr-NH2 |
| I-399 | Ac-PL3-Asp-Npg-B5-Asp-3COOHF-Aib-Ala-Phe-Lys*3-PyrS2-3Thi-BztA-GlnR*3-Val-Ser- |
| | NH2 |
| I-400 | Ac-PL3-Asp-Npg-B5-Asp-3COOHF-Aib-Ala-Phe-Lys*3-PyrS2-3Thi-BztA-GlnR*3-Aib-Ser- |
| | NH2 |
| I-401 | Ac-PL3-Asp-Npg-B5-Asp-3COOHF-Aib-Ala-Phe-Lys*3-PyrS2-3Thi-BztA-GlnR*3-Thr-Ser- |
| | NH2 |
| I-402 | Ac-PL3-Asp-Npg-B5-Asp-3COOHF-Aib-Ala-Phe-Lys*3-PyrS2-3Thi-BztA-GlnR*3-Val-Ala- |
| | NH2 |
| I-403 | Ac-PL3-Asp-Npg-B5-Asp-3COOHF-Aib-Ala-Phe-Lys*3-PyrS2-3Thi-BztA-GlnR*3-Val-Ala- |
| | NH2 |
| I-404 | Ac-PL3-Asp-Npg-B5-Asp-3COOHF-Aib-Ala-Phe-Lys*3-PyrS2-3Thi-BztA-GlnR*3-Aib-Ala- |
| | NH2 |
| I-405 | Ac-PL3-Asp-Npg-B5-Asp-3COOHF-Aib-Ala-Phe-Lys*3-PyrS2-3Thi-BztA-GlnR*3-Aib-Ala- |
| | NH2 |
| I-406 | Ac-PL3-Asp-Npg-B5-Asp-3COOHF-Aib-Ala-Phe-Lys*3-PyrS2-3Thi-BztA-GlnR*3-Thr-Ala- |
| | NH2 |
| I-407 | Ac-PL3-Asp-Npg-B5-Asp-3COOHF-Aib-Ala-Phe-Lys*3-PyrS2-3Thi-BztA-GlnR*3-Val-Aib- |
| | NH2 |
| I-408 | Ac-PL3-Asp-Npg-B5-Asp-3COOHF-Aib-Ala-Phe-Lys*3-PyrS2-3Thi-BztA-GlnR*3-Val-Aib- |
| | NH2 |
| I-409 | Ac-PL3-Asp-Npg-B5-Asp-3COOHF-Aib-Ala-Phe-Lys*3-PyrS2-3Thi-BztA-GlnR*3-Aib-Aib- |
| | NH2 |
| I-410 | Ac-PL3-Asp-Npg-B5-Asp-3COOHF-Aib-Ala-Phe-Lys*3-PyrS2-3Thi-BztA-GlnR*3-Aib-Aib- |
| | NH2 |
| I-411 | Ac-PL3-Asp-Npg-B5-Asp-3COOHF-Aib-Ala-Phe-Lys*3-PyrS2-3Thi-BztA-GlnR*3-Thr-Aib- |
| | NH2 |
| I-412 | Ac-PL3-Asp-Npg-B5-Asp-3COOHF-Aib-Ala-Phe-Lys*3-PyrS2-3Thi-BztA-GlnR*3-Thr-Aib- |
| | NH2 |
| I-413 | Ac-PL3-Asp-Npg-B5-Asp-3COOHF-Aib-Ala-Phe-TriAzLys*3-PyrS2-3Thi-BztA-sAla*3-Val- |
| | NH2 |
| I-414 | Ac-PL3-Asp-Npg-B5-Asp-3COOHF-Aib-Ala-Phe-TriAzLys*3-PyrS2-3Thi-BztA-sAla*3-Leu- |
| | NH2 |
| I-415 | Ac-PL3-Asp-Npg-B5-Asp-3COOHF-Aib-Ala-Phe-TriAzLys*3-PyrS2-3Thi-BztA-sAla*3-Thr- |
| | NH2 |
| I-416 | Ac-PL3-Asp-Npg-B5-Asp-3COOHF-Aib-Ala-Phe-TriAzLys*3-PyrS2-3Thi-BztA-sAla*3-Ala- |
| | Ser-NH2 |
| I-417 | Ac-PL3-Asp-Npg-B5-Asp-3COOHF-Aib-Ala-Phe-TriAzLys*3-PyrS2-3Thi-BztA-sAla*3-Val- |
| | Ser-NH2 |
| I-418 | Ac-PL3-Asp-Npg-B5-Asp-3COOHF-Aib-Ala-Phe-TriAzLys*3-PyrS2-Phe-BztA-sAla*3-Ala- |
| | NH2 |
| I-419 | Ac-PL3-Asp-Npg-B5-Asp-3COOHF-Aib-Ala-Phe-TriAzLys*3-PyrS2-Phe-BztA-sAbu*3-Ala- |
| | NH2 |
| I-420 | Ac-PL3-Asp-Npg-B5-Asp-3COOHF-Aib-Ala-Phe-TriAzLys*3-PyrS2-Phe-BztA-sAbu*3-Ala- |
| | NH2 |
| I-421 | Ac-PL3-Asp-Npg-B5-Asp-3COOHF-Aib-Ala-Phe-TriAzOrn*3-PyrS2-3Thi-BztA-sAla*3-Ala- |
| | NH2 |
| I-422 | Ac-PL3-Asp-Npg-B5-Asp-3COOHF-Aib-Ala-Phe-TriAzOrn*3-PyrS2-Phe-BztA-sAla*3-Ala- |
| | NH2 |
| I-423 | Ac-PL3-Asp-Npg-B5-Asp-3COOHF-Aib-Ala-Phe-TriAzOrn*3-PyrS2-3Thi-BztA-sAbu*3-Ala- |
| | NH2 |
| I-424 | Ac-PL3-Asp-Npg-B5-Asp-3COOHF-Aib-Ala-Phe-TriAzOrn*3-PyrS2-Phe-BztA-sAbu*3-Ala- |
| | NH2 |
| I-425 | 4penteny1-MePro-Asp-AllylGly-R5-Asp-3COOHF-Aib-Ala-Phe-Lys*3-PyrS2-3Thi-BztA- |
| | GlnR*3-Ala-NH2 |
| I-426 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-427 | 4penteny1-MePro-Asp-AllylGly-R5-Asp-3COOHF-Aib-Ala-Phe-Lys*3-PyrS2-3Thi-BztA- |
| | GlnR*3-Ala-NH2 |
| I-428 | 4pentenyl-MePro-Asp-AllylGly-R5-Asp-3COOHF-Aib-Ala-Phe-Lys*3-PyrS2-3Thi-BztA- |
| | GlnR*3-Ala-NH2 |
| I-429 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-430 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-431 | 5hexenyl-MePro-Asp-AllylGly-R5-Asp-3COOHF-Aib-Ala-Phe-Lys*3-PyrS2-3Thi-BztA- |
| | GlnR*3-Ala-NH2 |
| I-432 | Ac-PL3-Asp-Npg-B5-Asp-3COOHF-aMeL-Ala-Phe-Lys*3-PyrS2-3Thi-BztA-GlnR*3-Ala-NH2 |
| I-433 | Ac-PL3-Asp-Npg-B5-Asp-3COOHF-DaMeL-Ala-Phe-Lys*3-PyrS2-3Thi-BztA-GlnR*3-Ala- |
| | NH2 |
| I-434 | Ac-PL3-Asp-Npg-B5-Asp-3COOHF-aMeV-Ala-Phe-Lys*3-PyrS2-3Thi-BztA-GlnR*3-Ala-NH2 |
| I-435 | Ac-PL3-Asp-Npg-B5-Asp-3COOHF-aMeS-Ala-Phe-Lys*3-PyrS2-3Thi-BztA-GlnR*3-Ala-NH2 |
| I-436 | Ac-PL3-Asp-Npg-B5-Asp-3COOHF-DaMeS-Ala-Phe-Lys*3-PyrS2-3Thi-BztA-GlnR*3-Ala-NH2 |
| I-437 | Ac-PL3-Asp-Npg-B5-Asp-3COOHF-DaMeS-Ala-Phe-Lys*3-PyrS2-3Thi-BztA-GlnR*3-Ala-NH2 |
| I-438 | Ac-PL3-Asp-Npg-B5-Asp-3COOHF-aMeF-Ala-Phe-Lys*3-PyrS2-3Thi-BztA-GlnR*3-Ala-NH2 |
| I-439 | Ac-PL3-Asp-Npg-B5-Asp-3COOHF-Aib-Aib-Phe-Lys*3-PyrS2-3Thi-BztA-GlnR*3-Ala-NH2 |
| I-440 | Ac-PL3-Asp-Npg-B5-Asp-3COOHF-Aib-Cpg-Phe-Lys*3-PyrS2-3Thi-BztA-GlnR*3-Ala-NH2 |
| I-441 | Ac-PL3-Asp-Npg-B5-Asp-3COOHF-Ala-Aib-Phe-Lys*3-PyrS2-3Thi-BztA-GlnR*3-Ala-NH2 |
| I-442 | Ac-PL3-Asp-Npg-B5-Asp-3COOHF-Ala-Aib-Phe-Lys*3-PyrS2-3Thi-BztA-GlnR*3-Ala-NH2 |
| I-443 | Ac-PL3-Asp-Npg-B5-Asp-3COOHF-Ala-Cpg-Phe-Lys*3-PyrS2-3Thi-BztA-GlnR*3-Ala-NH2 |
| I-444 | Ac-PL3-Asp-Npg-B5-Asp-3COOHF-Aib-Ser-Phe-Lys*3-PyrS2-3Thi-BztA-GlnR*3-Ala-NH2 |
| I-445 | Ac-PL3-Asp-Npg-B5-Asp-3COOHF-Aib-Thr-Phe-Lys*3-PyrS2-3Thi-BztA-GlnR*3-Ala-NH2 |
| I-446 | Ac-PL3-Asp-Npg-B5-Asp-3COOHF-Aib-aThr-Phe-Lys*3-PyrS2-3Thi-BztA-GlnR*3-Ala-NH2 |
| I-447 | Ac-PL3-Asp-Npg-B5-Asp-3COOHF-Aib-Val-Phe-Lys*3-PyrS2-3Thi-BztA-GlnR*3-Ala-NH2 |
| I-448 | Ac-PL3-Asp-Npg-B5-Asp-3COOHF-Aib-Val-Phe-Lys*3-PyrS2-3Thi-BztA-GlnR*3-Ala-NH2 |
| I-449 | Ac-PL3-Asp-Npg-B5-Asp-3COOHF-Aib-Leu-Phe-Lys*3-PyrS2-3Thi-BztA-GlnR*3-Ala-NH2 |
| I-450 | Ac-PL3-Asp-Npg-B5-Asp-3COOHF-Aib-Leu-Phe-Lys*3-PyrS2-3Thi-BztA-GlnR*3-Ala-NH2 |
| I-451 | Ac-PL3-Asp-Npg-B5-Asp-3COOHF-Aib-Gln-Phe-Lys*3-PyrS2-3Thi-BztA-GlnR*3-Ala-NH2 |
| I-452 | Ac-PL3-Asp-Npg-B5-Asp-3COOHF-Aib-Gln-Phe-Lys*3-PyrS2-3Thi-BztA-GlnR*3-Ala-NH2 |
| I-453 | Ac-PL3-Asp-Npg-B5-Asp-3COOHF-Aib-MorphGln-Phe-Lys*3-PyrS2-3Thi-BztA-GlnR*3-Ala- |
| | NH2 |
| I-454 | Ac-PL3-Asp-Npg-B5-Asp-3COOHF-Aib-Lys-Phe-Lys*3-PyrS2-3Thi-BztA-GlnR*3-Ala-NH2 |
| I-455 | Ac-PL3-Asp-Npg-B5-Asp-3COOHF-Aib-Lys-Phe-Lys*3-PyrS2-3Thi-BztA-GlnR*3-Ala-NH2 |
| I-456 | Ac-PL3-Asp-Npg-B5-Asp-3COOHF-aMeDF-Ala-Phe-Lys*3-PyrS2-3Thi-BztA-GlnR*3-Ala-NH2 |
| I-457 | Ac-PL3-Asp-Npg-B5-Asp-3COOHF-aMeDF-Ala-Phe-Lys*3-PyrS2-3Thi-BztA-GlnR*3-Ala-NH2 |
| I-458 | Ac-PL3-Asp-Npg-B5-Asp-3COOHF-Aib-Ala-Phe-Lys*3-PyrS2-3Thi-BztA-GlnR*3-dAla-NH2 |
| I-459 | Ac-PL3-Asp-Npg-B5-Asp-3COOHF-Aib-Ala-Phe-Lys*3-PyrS2-3Thi-BztA-GlnR*3-dAla-NH2 |
| I-460 | Ac-PL3-Asp-Npg-B5-Asp-3COOHF-Aib-Ala-Phe-Lys*3-PyrS2-3Thi-BztA-GlnR*3-dAla-Ser- |
| | NH2 |
| I-461 | Ac-PL3-Asp-Npg-B5-Asp-3COOHF-Aib-Ala-Phe-Lys*3-PyrS2-3Thi-BztA-GlnR*3-dAla-Ser- |
| | NH2 |
| I-462 | Ac-PL3-Asp-Npg-B5-Asp-3COOHF-Aib-Ala-Phe-Lys*3-PyrS2-3Thi-BztA-GlnR*3-dLeu-NH2 |
| I-463 | Ac-PL3-Asp-Npg-B5-Asp-3COOHF-Aib-Ala-Phe-Lys*3-PyrS2-3Thi-BztA-GlnR*3-dLeu-Ser- |
| | NH2 |
| I-464 | Ac-PL3-Asp-Npg-B5-Asp-3COOHF-Aib-Ala-Phe-Lys*3-PyrS2-3Thi-BztA-GlnR*3-Ala-Ser- |
| | Leu-NH2 |
| I-465 | Ac-PL3-Asp-Npg-B5-Asp-3COOHF-Aib-Ala-Phe-Lys*3-PyrS2-3Thi-BztA-GlnR*3-Leu-Ser- |
| | Leu-NH2 |
| I-466 | Ac-PL3-Asp-Npg-B5-Asp-3COOHF-Aib-Ala-Phe-Lys*3-PyrS2-3Thi-BztA-GlnR*3-Leu-Ser- |
| | Leu-NH2 |
| I-467 | Ac-PL3-Asp-Npg-B5-Asp-3COOHF-Aib-Ala-Phe-Lys*3-PyrS2-3Thi-BztA-GlnR*3-dLeu-Ser- |
| | Leu-NH2 |
| I-468 | Ac-PL3-Asp-Npg-B5-Asp-3COOHF-Aib-Ala-Phe-Lys*3-PyrS2-3Thi-BztA-GlnR*3-dLeu-Ser- |
| | Leu-NH2 |
| I-469 | Ac-PL3-OAsp-Npg-B5-Asp-3COOHF-Aib-Ala-Phe-Lys*3-PyrS2-2F3MeF-BztA-GlnR*3-Ala- |
| | NH2 |
| I-470 | Ac-PL3-Glu-Npg-B5-Glu-3COOHF-Aib-Ala-Phe-Lys*3-PyrS2-3Thi-BztA-GlnR*3-Ala-NH2 |
| I-471 | BzAm20Allyl-MePro-Asp-AllylGly-R5-Asp-3COOHF-Aib-Ala-Phe-Lys*3-PyrS2-3Thi-BztA- |
| | GlnR*3-Ala-NH2 |
| I-472 | Ac-PL3-Asp-Npg-B5-Asp-3COOHF-Aib-Ala-Phe-Lys*3-PyrS2-2ClF-34ClF-GlnR*3-Ala-NH2 |
| I-473 | Ac-PL3-Asp-Npg-B5-Asp-3COOHF-Aib-Ala-Phe-Lys*3-PyrS2-nLeu-34ClF-GlnR*3-Ala-NH2 |
| I-474 | Ac-PL3-Asp-Npg-B5-Asp-3COOHF-Aib-Ala-Phe-Lys*3-PyrS2-nLeu-34ClF-GlnR*3-Ala-NH2 |
| I-475 | Ac-PL3-Asp-Npg-B5-Asp-3COOHF-Aib-Ala-Phe-Lys*3-PyrS2-CypA-34ClF-GlnR*3-Ala-NH2 |
| I-476 | Ac-PL3-Asp-Npg-B5-Asp-3COOHF-Aib-Ala-Phe-Lys*3-PyrS2-CypA-34ClF-GlnR*3-Ala-NH2 |
| I-477 | Ac-PL3-Asp-Npg-B5-Asp-3COOHF-Aib-Ala-Tyr-Lys*3-PyrS2-Phe-34ClF-GlnR*3-Ala-NH2 |
| I-478 | Ac-PL3-Asp-Npg-B5-Asp-3COOHF-Aib-Ala-3Thi-Lys*3-PyrS2-Phe-34ClF-GlnR*3-Ala-NH2 |
| I-479 | Ac-PL3-Asp-Npg-B5-Asp-3COOHF-Aib-Ala-nLeu-Lys*3-PyrS2-Phe-34ClF-GlnR*3-Ala-NH2 |
| I-480 | Ac-PL3-Asp-Npg-B5-Asp-3COOHF-Aib-Ala-nLeu-Lys*3-PyrS2-Phe-34ClF-GlnR*3-Ala-NH2 |
| I-481 | Ac-PL3-Asp-Npg-B5-Asp-3COOHF-Aib-Ala-2NapA-Lys*3-PyrS2-Phe-34ClF-GlnR*3-Ala-NH2 |
| I-482 | Ac-PL3-Asp-Npg-B5-Asp-3COOHF-Aib-Ala-2NapA-Lys*3-PyrS2-Phe-34ClF-GlnR*3-Ala-NH2 |
| I-483 | Ac-PL3-Asp-Npg-B5-Asp-3COOHF-Aib-Ala-Phe-Lys*3-PyrS2-Phe-2NapA-GlnR*3-Ala-NH2 |
| I-484 | Ac-PL3-Asp-Npg-B5-Asp-3COOHF-Aib-Ala-Phe-Lys*3-PyrS2-Phe-2NapA-GlnR*3-Ala-NH2 |
| I-485 | Ac-PL3-Asp-Npg-B5-Asp-3COOHF-Aib-Ala-Phe-Lys*3-PyrS2-3Thi-2NapA-GlnR*3-Ala-NH2 |
| I-486 | Ac-PL3-Asp-Npg-B5-Asp-3COOHF-Aib-Ala-Phe-Lys*3-PyrS2-3Thi-2NapA-GlnR*3-Ala-NH2 |
| I-487 | Ac-PL3-Asp-Npg-B5-Asp-3COOHF-Aib-Ala-Phe-Lys*3-PyrS2-2ClF-2NapA-GlnR*3-Ala-NH2 |
| I-488 | Ac-PL3-Asp-Npg-B5-Asp-3COOHF-Aib-Ala-Phe-Lys*3-PyrS2-2ClF-2NapA-GlnR*3-Ala-NH2 |
| I-489 | Ac-PL3-Asp-Npg-B5-Asp-3COOHF-Aib-Ala-Phe-Lys*3-PyrS2-nLeu-2NapA-GlnR*3-Ala-NH2 |
| I-490 | Ac-PL3-Asp-Npg-B5-Asp-3COOHF-Aib-Ala-Phe-Lys*3-PyrS2-nLeu-2NapA-GlnR*3-Ala-NH2 |
| I-491 | Ac-PL3-Asp-Npg-B5-Asp-3COOHF-Aib-Ala-Phe-Lys*3-PyrS2-CypA-2NapA-GlnR*3-Ala-NH2 |
| I-492 | Ac-PL3-Asp-Npg-B5-Asp-3COOHF-Aib-Ala-Phe-Lys*3-PyrS2-CypA-2NapA-GlnR*3-Ala-NH2 |
| I-493 | Ac-PL3-Asp-Npg-B5-Asp-3COOHF-Aib-Ala-Phe-Lys*3-PyrS2-3Thi-BztA-GlnR*3-Leu-Asp- |
| | Ala-NH2 |
| I-494 | Ac-PL3-Asp-Npg-B5-Asp-3COOHF-Aib-Ala-Phe-Lys*3-PyrS2-3Thi-BztA-GlnR*3-Leu-Glu- |
| | Ala-NH2 |
| I-495 | Ac-PL3-Asp-Npg-B5-Asp-3COOHF-Aib-Ala-Phe-Lys*3-PyrS2-3Thi-BztA-GlnR*3-Leu-Glu- |
| | Ala-NH2 |
| I-496 | Ac-PL3-Asp-Npg-B5-Asp-3COOHF-Aib-Ala-Phe-Lys*3-PyrS2-3Thi-BztA-GlnR*3-Leu-Ser- |
| | Ala-NH2 |
| I-497 | Ac-PL3-Asp-Npg-B5-Asp-3COOHF-Aib-Ala-Phe-Lys*3-PyrS2-3Thi-BztA-GlnR*3-Leu-Ser- |
| | Ala-NH2 |
| I-498 | Ac-PL3-Asp-Npg-B5-Asp-3COOHF-Aib-Ala-Phe-Lys*3-PyrS2-3Thi-BztA-GlnR*3-Leu-Thr- |
| | Ala-NH2 |
| I-499 | Ac-PL3-Asp-Npg-B5-Asp-3COOHF-Aib-Ala-Phe-Lys*3-PyrS2-3Thi-BztA-GlnR*3-Leu-Thr- |
| | Ala-NH2 |
| I-500 | Ac-PL3-Asp-Npg-B5-Asp-3COOHF-Aib-Ala-Phe-Lys*3-PyrS2-3Thi-BztA-GlnR*3-Leu-aThr- |
| | Ala-NH2 |
| I-501 | Ac-PL3-Asp-Npg-B5-Asp-3COOHF-Aib-Ala-Phe-Lys*3-PyrS2-3Thi-BztA-GlnR*3-Leu-aThr- |
| | Ala-NH2 |
| I-502 | Ac-PL3-Asp-Npg-B5-Asp-3COOHF-Aib-Ala-Phe-Lys*3-PyrS2-3Thi-BztA-GlnR*3-Leu-Asp- |
| | Leu-NH2 |
| I-503 | Ac-PL3-Asp-Npg-B5-Asp-3COOHF-Aib-Ala-Phe-Lys*3-PyrS2-3Thi-BztA-GlnR*3-Leu-Asp- |
| | Leu-NH2 |
| I-504 | Ac-PL3-Asp-Npg-B5-Asp-3COOHF-Aib-Ala-Phe-Lys*3-PyrS2-3Thi-BztA-GlnR*3-Leu-Glu- |
| | Leu-NH2 |
| I-505 | Ac-PL3-Asp-Npg-B5-Asp-3COOHF-Aib-Ala-Phe-Lys*3-PyrS2-3Thi-BztA-GlnR*3-Leu-Glu- |
| | Leu-NH2 |
| I-506 | Ac-PL3-Asp-Npg-B5-Asp-3COOHF-Aib-Ala-Phe-Lys*3-PyrS2-3Thi-BztA-GlnR*3-Val-Asp- |
| | Ala-NH2 |
| I-507 | Ac-PL3-Asp-Npg-B5-Asp-3COOHF-Aib-Ala-Phe-Lys*3-PyrS2-3Thi-BztA-GlnR*3-Val-Glu- |
| | Ala-NH2 |
| I-508 | Ac-PL3-Asp-Npg-B5-Asp-3COOHF-Aib-Ala-Phe-Lys*3-PyrS2-3Thi-BztA-GlnR*3-Val-Thr- |
| | Ala-NH2 |
| I-509 | Ac-PL3-Asp-Npg-B5-Asp-3COOHF-Aib-Ala-Phe-Lys*3-PyrS2-3Thi-BztA-GlnR*3-Val-Thr- |
| | Ala-NH2 |
| I-510 | Ac-PL3-Asp-Npg-B5-Asp-3COOHF-Aib-Ala-Phe-Lys*3-PyrS2-3Thi-BztA-GlnR*3-Val-aThr- |
| | Ala-NH2 |
| I-511 | Ac-PL3-Asp-Npg-B5-Asp-3COOHF-Aib-Ala-Phe-Lys*3-PyrS2-3Thi-BztA-GlnR*3-Val-aThr- |
| | Ala-NH2 |
| I-512 | Ac-PL3-Asp-Npg-B5-Asp-3COOHF-Aib-Ala-Phe-Lys*3-PyrS2-3Thi-BztA-GlnR*3-Val-Asp- |
| | Leu-NH2 |
| I-513 | Ac-PL3-Asp-Npg-B5-Asp-3COOHF-Aib-Ala-Phe-Lys*3-PyrS2-3Thi-BztA-GlnR*3-Val-Asp- |
| | Leu-NH2 |
| I-514 | Ac-Gly-PL3-Asp-Npg-B5-Asp-3COOHF-Aib-Ala-Phe-Lys*3-PyrS2-3Thi-BztA-GlnR*3-Ala |
| I-515 | Ac-Sar-PL3-Asp-Npg-B5-Asp-3COOHF-Aib-Ala-Phe-Lys*3-PyrS2-3Thi-BztA-GlnR*3-Ala- |
| | NH2 |
| I-516 | Ac-NMebAla-PL3-Asp-Npg-B5-Asp-3COOHF-Aib-Ala-Phe-Lys*3-PyrS2-3Thi-BztA-GlnR*3- |
| | Ala-NH2 |
| I-517 | Ac-PL3-Asp-Npg-B5-Asp-3COOHF-Aib-Ala-Phe-Lys*3-PyrS2-3Thi-BztA-GlnR*3- |
| | [BiotinPEG8]Lys-NH2 |
| I-518 | Ac-PL3-Glu-Npg-B5-Glu-3COOHF-Aib-Ala-Phe-Lys*3-PyrS2-3Thi-BztA-GlnR*3- |
| | [BiotinPEG8]Lys-NH2 |
| I-519 | 5hexenyl-MePro-Asp-[Bn][Allyl]Dap-R5-Asp-3COOHF-Aib-Ala-Phe-Lys*3-PyrS2-3Thi-BztA- |
| | GlnR*3-Ala-NH2 |
| I-520 | 5hexenyl-MePro-Asp-[Phc][Allyl]Dap-R5-Asp-3COOHF-Aib-Ala-Phe-Lys*3-PyrS2-3Thi-BztA- |
| | GlnR*3-Ala-NH2 |
| I-521 | Ac-PL3-Asp-Npg-B5-Asp-3COOHF-Aib-Asp-Phe-Lys*3-PyrS2-3Thi-BztA-GlnR*3-Ala-NH2 |
| I-522 | Ac-PL3-Asp-Npg-B5-aThr-3COOHF-Aib-Asp-Phe-Lys*3-PyrS2-3Thi-BztA-GlnR*3-Ala-NH2 |
| I-523 | Ac-PL3-Asp-Npg-B5-Asp-3COOHF-Aib-Glu-Phe-Lys*3-PyrS2-3Thi-BztA-GlnR*3-Ala-NH2 |
| I-524 | Ac-PL3-Asp-Npg-B5-aThr-3COOHF-Aib-Glu-Phe-Lys*3-PyrS2-3Thi-BztA-GlnR*3-Ala-NH2 |
| I-525 | Ac-PL3-Asp-Npg-B5-Asp-3COOHF-Aib-Aad-Phe-Lys*3-PyrS2-3Thi-BztA-GlnR*3-Ala-NH2 |
| I-526 | Ac-PL3-Asp-Npg-B5-aThr-3COOHF-Aib-Aad-Phe-Lys*3-PyrS2-3Thi-BztA-GlnR*3-Ala-NH2 |
| I-527 | Ac-PL3-Asp-Npg-B5-Asp-3COOHF-Aib-nLeu-Phe-Lys*3-PyrS2-3Thi-BztA-GlnR*3-Ala-NH2 |
| I-528 | Ac-PL3-Asp-Npg-B5-Asp-3COOHF-Aib-nLeu-Phe-Lys*3-PyrS2-3Thi-BztA-GlnR*3-Ala-NH2 |
| I-529 | Ac-PL3-Asp-Npg-B5-Asp-3COOHF-Aib-Phe-Phe-Lys*3-PyrS2-3Thi-BztA-GlnR*3-Ala-NH2 |
| I-530 | Ac-PL3-Asp-Npg-B5-Asp-3COOHF-Aib-hPhe-Phe-Lys*3-PyrS2-3Thi-BztA-GlnR*3-Ala-NH2 |
| I-531 | Ac-PL3-Asp-Npg-B5-Asp-3COOHF-Aib-Cba-Phe-Lys*3-PyrS2-3Thi-BztA-GlnR*3-Ala-NH2 |
| I-532 | Ac-PL3-Asp-Npg-B5-Asp-3COOHF-Aib-Cba-Phe-Lys*3-PyrS2-3Thi-BztA-GlnR*3-Ala-NH2 |
| I-533 | Ac-PL3-Asp-Npg-B5-Asp-3COOHF-Aib-hTyr-Phe-Lys*3-PyrS2-3Thi-BztA-GlnR*3-Ala-NH2 |
| I-534 | Ac-PL3-Asp-Npg-B5-Asp-3COOHF-Aib-AcLys-Phe-Lys*3-PyrS2-3Thi-BztA-GlnR*3-Ala-NH2 |
| I-535 | Ac-PL3-Asp-Npg-B5-Asp-3COOHF-Aib-Ala-Phe-Lys*3-PyrS2-Val-BztA-GlnR*3-Ala-NH2 |
| I-536 | Ac-PL3-Asp-Npg-B5-Asp-3COOHF-Aib-Ala-Phe-Lys*3-PyrS2-Ile-BztA-GlnR*3-Ala-NH2 |
| I-537 | Ac-PL3-Asp-Npg-B5-Asp-3COOHF-Aib-Ala-Phe-Lys*3-PyrS2-Chg-BztA-GlnR*3-Ala-NH2 |
| I-538 | Ac-PL3-Asp-Npg-B5-Asp-3COOHF-Aib-Ala-Phe-Lys*3-PyrS2-DiethA-BztA-GlnR*3-Ala-NH2 |
| I-539 | Ac-PL3-Asp-Npg-B5-Asp-3COOHF-Aib-Ala-Phe-Lys*3-PyrS2-hnLeu-BztA-GlnR*3-Ala-NH2 |
| I-540 | Ac-PL3-Asp-Npg-B5-Asp-3COOHF-Aib-Ala-Phe-Lys*3-PyrS2-OctG-BztA-GlnR*3-Ala-NH2 |
| I-541 | Ac-PL3-Asp-Npg-B5-Asp-3COOHF-Aib-Ala-Phe-Lys*3-PyrS2-2Thi-BztA-GlnR*3-Ala-NH2 |
| I-542 | Ac-PL3-Asp-Npg-B5-Asp-3COOHF-Aib-Ala-Phe-Lys*3-PyrS2-2cbmF-BztA-GlnR*3-Ala-NH2 |
| I-543 | Ac-PL3-Glu-Npg-B5-Asp-3COOHF-Aib-Ala-Phe-Lys*3-PyrS2-3Thi-BztA-GlnR*3-Ala-NH2 |
| I-544 | Ac-PL3-Glu-Npg-B5-Asp-3COOHF-Aib-Ala-Phe-Lys*3-PyrS2-3Thi-BztA-GlnR*3-Ala-NH2 |
| I-545 | Ac-PL3-Aad-Npg-B5-Asp-3COOHF-Aib-Ala-Phe-Lys*3-PyrS2-3Thi-BztA-GlnR*3-Ala-NH2 |
| I-546 | Ac-PL3-Aad-Npg-B5-Asp-3COOHF-Aib-Ala-Phe-Lys*3-PyrS2-3Thi-BztA-GlnR*3-Ala-NH2 |
| I-547 | Ac-PL3-Asp-Npg-B5-Glu-3COOHF-Aib-Ala-Phe-Lys*3-PyrS2-3Thi-BztA-GlnR*3-Ala-NH2 |
| I-548 | Ac-PL3-Asp-Npg-B5-Aad-3COOHF-Aib-Ala-Phe-Lys*3-PyrS2-3Thi-BztA-GlnR*3-Ala-NH2 |
| I-549 | Ac-PL3-Aad-Npg-B5-Glu-3COOHF-Aib-Ala-Phe-Lys*3-PyrS2-3Thi-BztA-GlnR*3-Ala-NH2 |
| I-550 | Ac-PL3-Glu-Npg-B5-Aad-3COOHF-Aib-Ala-Phe-Lys*3-PyrS2-3Thi-BztA-GlnR*3-Ala-NH2 |
| I-551 | Ac-PL3-Aad-Npg-B5-Aad-3COOHF-Aib-Ala-Phe-Lys*3-PyrS2-3Thi-BztA-GlnR*3-Ala-NH2 |
| I-552 | Ac-PL3-Glu-Npg-B5-Glu-Glu-Aib-Ala-Phe-Lys*3-PyrS2-3Thi-BztA-GlnR*3-Ala-NH2 |
| I-553 | Ac-PL3-Aad-Npg-B5-Glu-Glu-Aib-Ala-Phe-Lys*3-PyrS2-3Thi-BztA-GlnR*3-Ala-NH2 |
| I-554 | Ac-PL3-Glu-Npg-B5-Glu-Asp-Aib-Ala-Phe-Lys*3-PyrS2-3Thi-BztA-GlnR*3-Ala-NH2 |
| I-555 | Ac-PL3-Aad-Npg-B5-Glu-Asp-Aib-Ala-Phe-Lys*3-PyrS2-3Thi-BztA-GlnR*3-Ala-NH2 |
| I-556 | Ac-PL3-Aad-Npg-B5-Aad-Glu-Aib-Ala-Phe-Lys*3-PyrS2-3Thi-BztA-GlnR*3-Ala-NH2 |
| I-557 | Ac-PL3-Asp-Npg-B5-Asp-3COOHF-Aib-Ala-Phe-dLys*3-PyrS2-3Thi-BztA-dGlnR*3-Ala-NH2 |
| I-558 | Ac-PL3-Asp-Npg-B5-Asp-3COOHF-Aib-Ala-Phe-dOrn*3-PyrS2-3Thi-BztA-GlnR*3-Ala-NH2 |
| I-559 | Ac-PL3-Asp-Npg-B5-Asp-3COOHF-Aib-Ala-Phe-dDab*3-PyrS2-3Thi-BztA-GlnR*3-Ala-NH2 |
| I-560 | Ac-PL3-Asp-Npg-B5-Asp-3COOHF-Aib-Ala-Phe-Orn*3-PyrS2-3Thi-BztA-hGlnR*3-Ala-NH2 |
| I-561 | Ac-PL3-Asp-Npg-B5-Asp-3COOHF-Aib-Ala-Phe-NMeOrn*3-PyrS2-3Thi-BztA-hGlnR*3-Ala- |
| | NH2 |
| I-562 | 4pentenyl-MePro-Asp-[Bn][Allyl]Dap-R5-Asp-3COOHF-Aib-Ala-Phe-Lys*3-PyrS2-3Thi-BztA- |
| | GlnR*3-Ala-NH2 |
| I-563 | 4pentenyl-MePro-Asp-[Phc][Allyl]Dap-R5-Asp-3COOHF-Aib-Ala-Phe-Lys*3-PyrS2-3Thi-BztA- |
| | GlnR*3-Ala-NH2 |
| I-564 | 5hexenyl-MePro-Asp-[Piv][Allyl]Dap-R5-Asp-3COOHF-Aib-Ala-Phe-Lys*3-PyrS2-3Thi-BztA- |
| | GlnR*3-Ala-NH2 |
| I-565 | 5hexenyl-MePro-Asp-[CyCO][Allyl]Dap-R5-Asp-3COOHF-Aib-Ala-Phe-Lys*3-PyrS2-3Thi- |
| | BztA-GlnR*3-Ala-NH2 |
| I-566 | Ac-PL3-Asp-Npg-B5-Asp-3COOHF-Aib-Ala-Phe-[2_6-naph]Cys-PyrS2-3Thi-BztA-Cys-Ala- |
| | NH2 |
| I-567 | Ac-PL3-Asp-Npg-B5-Asp-3COOHF-Aib-Ala-Phe-[3_3-biph]Cys-PyrS2-3Thi-BztA-Cys-Ala- |
| | NH2 |
| I-568 | Ac-PL3-Asp-Npg-B5-Asp-3COOHF-Aib-Ala-4FF-Lys*3-PyrS2-3Thi-BztA-GlnR*3-Ala-NH2 |
| I-569 | Ac-PL3-Asp-Npg-B5-Asp-3COOHF-Aib-Ala-4FF-Lys*3-PyrS2-3Thi-BztA-GlnR*3-Ala-NH2 |
| I-570 | Ac-PL3-Asp-Npg-B5-Asp-3COOHF-Aib-Ala-4ClF-Lys*3-PyrS2-3Thi-BztA-GlnR*3-Ala-NH2 |
| I-571 | Ac-PL3-Asp-Npg-B5-Asp-3COOHF-Aib-Ala-4ClF-Lys*3-PyrS2-3Thi-BztA-GlnR*3-Ala-NH2 |
| I-572 | Ac-PL3-Asp-Npg-B5-Asp-3COOHF-Aib-Ala-4BrF-Lys*3-PyrS2-3Thi-BztA-GlnR*3-Ala-NH2 |
| I-573 | Ac-PL3-Asp-Npg-B5-Asp-3COOHF-Aib-Ala-4BrF-Lys*3-PyrS2-3Thi-BztA-GlnR*3-Ala-NH2 |
| I-574 | Ac-PL3-Asp-Npg-B5-Asp-3COOHF-Aib-Ala-3FF-Lys*3-PyrS2-3Thi-BztA-GlnR*3-Ala-NH2 |
| I-575 | Ac-PL3-Asp-Npg-B5-Asp-3COOHF-Aib-Ala-3FF-Lys*3-PyrS2-3Thi-BztA-GlnR*3-Ala-NH2 |
| I-576 | Ac-PL3-Asp-Npg-B5-Asp-3COOHF-Aib-Ala-3ClF-Lys*3-PyrS2-3Thi-BztA-GlnR*3-Ala-NH2 |
| I-577 | Ac-PL3-Asp-Npg-B5-Asp-3COOHF-Aib-Ala-3ClF-Lys*3-PyrS2-3Thi-BztA-GlnR*3-Ala-NH2 |
| I-578 | Ac-PL3-Asp-Npg-B5-Asp-3COOHF-Aib-Ala-3BrF-Lys*3-PyrS2-3Thi-BztA-GlnR*3-Ala-NH2 |
| I-579 | Ac-PL3-Asp-Npg-B5-Asp-3COOHF-Aib-Ala-3BrF-Lys*3-PyrS2-3Thi-BztA-GlnR*3-Ala-NH2 |
| I-580 | Ac-PL3-Asp-Npg-B5-Asp-3COOHF-Aib-Ala-2FF-Lys*3-PyrS2-3Thi-BztA-GlnR*3-Ala-NH2 |
| I-581 | Ac-PL3-Asp-Npg-B5-Asp-3COOHF-Aib-Ala-2FF-Lys*3-PyrS2-3Thi-BztA-GlnR*3-Ala-NH2 |
| I-582 | Ac-PL3-Asp-Npg-B5-Asp-3COOHF-Aib-Ala-30MeF-Lys*3-PyrS2-3Thi-BztA-GlnR*3-Ala- |
| | NH2 |
| I-583 | Ac-PL3-Asp-Npg-B5-Asp-3COOHF-Aib-Ala-30MeF-Lys*3-PyrS2-3Thi-BztA-GlnR*3-Ala- |
| | NH2 |
| I-584 | Ac-PL3-Asp-Npg-B5-Asp-3COOHF-Aib-Ala-4CNF-Lys*3-PyrS2-3Thi-BztA-GlnR*3-Ala-NH2 |
| I-585 | Ac-PL3-Asp-Npg-B5-Asp-3COOHF-Aib-Ala-4CNF-Lys*3-PyrS2-3Thi-BztA-GlnR*3-Ala-NH2 |
| I-586 | Ac-PL3-Asp-Npg-B5-Asp-3COOHF-Aib-Ala-3CNF-Lys*3-PyrS2-3Thi-BztA-GlnR*3-Ala-NH2 |
| I-587 | Ac-PL3-Asp-Npg-B5-Asp-3COOHF-Aib-Ala-3CNF-Lys*3-PyrS2-3Thi-BztA-GlnR*3-Ala-NH2 |
| I-588 | Ac-PL3-Asp-Npg-B5-Asp-3COOHF-Aib-Ala-4MeF-Lys*3-PyrS2-3Thi-BztA-GlnR*3-Ala-NH2 |
| I-589 | Ac-PL3-Asp-Npg-B5-Asp-3COOHF-Aib-Ala-4MeF-Lys*3-PyrS2-3Thi-BztA-GlnR*3-Ala-NH2 |
| I-590 | Ac-PL3-Asp-Npg-B5-Asp-3COOHF-Aib-Ala-3MeF-Lys*3-PyrS2-3Thi-BztA-GlnR*3-Ala-NH2 |
| I-591 | Ac-PL3-Asp-Npg-B5-Asp-3COOHF-Aib-Ala-3MeF-Lys*3-PyrS2-3Thi-BztA-GlnR*3-Ala-NH2 |
| I-592 | Ac-PL3-Asp-Npg-B5-Asp-3COOHF-Aib-Ala-Aic-Lys*3-PyrS2-3Thi-BztA-GlnR*3-Ala-NH2 |
| I-593 | Ac-PL3-Asp-Npg-B5-Asp-3COOHF-Aib-Ala-Aic-Lys*3-PyrS2-3Thi-BztA-GlnR*3-Ala-NH2 |
| I-594 | Ac-PL3-Asp-Npg-B5-Asp-3COOHF-Aib-Ala-RbiPrF-Lys*3-PyrS2-3Thi-BztA-GlnR*3-Ala-NH2 |
| I-595 | Ac-PL3-Asp-Npg-B5-Asp-3COOHF-Aib-Ala-RbiPrF-Lys*3-PyrS2-3Thi-BztA-GlnR*3-Ala-NH2 |
| I-596 | Ac-PL3-Asp-Npg-B5-Asp-3COOHF-Aib-Ala-SbiPrF-Lys*3-PyrS2-3Thi-BztA-GlnR*3-Ala-NH2 |
| I-597 | Ac-PL3-Asp-Npg-B5-Asp-3COOHF-Aib-Ala-SbiPrF-Lys*3-PyrS2-3Thi-BztA-GlnR*3-Ala-NH2 |
| I-598 | Ac-PL3-Asp-Npg-B5-Asp-3COOHF-Aib-Ala-RbiPrDF-Lys*3-PyrS2-3Thi-BztA-GlnR*3-Ala- |
| | NH2 |
| I-599 | Ac-PL3-Asp-Npg-B5-Asp-3COOHF-Aib-Ala-RbiPrDF-Lys*3-PyrS2-3Thi-BztA-GlnR*3-Ala- |
| | NH2 |
| I-600 | Ac-PL3-Asp-Npg-B5-Asp-3COOHF-Aib-Ala-RbMeXylA-Lys*3-PyrS2-3Thi-BztA-GlnR*3-Ala- |
| | NH2 |
| I-601 | Ac-PL3-Asp-Npg-B5-Asp-3COOHF-Aib-Ala-RbMeXylA-Lys*3-PyrS2-3Thi-BztA-GlnR*3-Ala- |
| | NH2 |
| I-602 | Ac-PL3-Asp-Npg-B5-Asp-3COOHF-Aib-Ala-RbMeXylDA-Lys*3-PyrS2-3Thi-BztA-GlnR*3- |
| | Ala-NH2 |
| I-603 | Ac-PL3-Asp-Npg-B5-Asp-3COOHF-Aib-Ala-RbMeXylDA-Lys*3-PyrS2-3Thi-BztA-GlnR*3- |
| | Ala-NH2 |
| I-604 | Ac-PL3-Asp-Npg-B5-Asp-3COOHF-Aib-Ala-SbMeXylA-Lys*3-PyrS2-3Thi-BztA-GlnR*3-Ala- |
| | NH2 |
| I-605 | Ac-PL3-Asp-Npg-B5-Asp-3COOHF-Aib-Ala-SbMeXylDA-Lys*3-PyrS2-3Thi-BztA-GlnR*3- |
| | Ala-NH2 |
| I-606 | Ac-PL3-Glu-Npg-B5-Glu-3COOHF-Aib-Ala-Phe-Lys*3-PyrS2-3Thi-BztA-GlnR*3-AzLys-NH2 |
| I-607 | Ac-PL3-Asp-Npg-B5-Asp-3COOHF-Aib-Ala-Phe-Lys*3-PyrS2-3Thi-BztA-GlnR*3-AzLys-NH2 |

Certain results from various additional assessment for various additional agents and compositions are presented in Table E3 below. See Table E1 and Table E2 for description. Among other things, these data confirm that technologies of the present disclosure can provide various activities and/or benefits.

TABLE E3
Certain data of various compositions as examples.

| 1 | 2 | 3 | 4 | 5 | 6 | 7 | 8 | 9 |
|---|---|---|---|---|---|---|---|---|
| I-608 | A | | | | 1956.8426 | | | |
| I-609 | A | | | | 1956.8426 | | | |
| I-700 | A | | | | 1956.8426 | | | |
| I-701 | B | | | | 2136.9519 | | | |
| I-702 | B | | | | 2164.9832 | 2166.5 | 2164.6 | |
| I-703 | B | | | | 2150.9675 | 2152.6 | 2150.6 | |
| I-704 | A | C | | | 2178.9988 | 2180.3 | 2178.4 | |
| I-705 | A | C | | | 2221.0458 | 2222.4 | 2220.5 | |
| I-706 | A | | | | 2060.9052 | | | |
| I-707 | A | | | | 2086.9209 | | | |
| I-708 | A | B | | | 2170.9209 | | | |
| I-709 | A | A | | + | 2084.9165 | | | |
| I-710 | A | B | | | 2102.9522 | 2104.9 | 2102.9 | |
| I-711 | A | B | | | 2088.9365 | 2090.8 | 2089.1 | |
| I-712 | A | A | | | 2003.8838 | 2005.7 | 2003.8 | |
| I-713 | A | | | | 2003.8838 | 2005.7 | 2003.9 | |
| I-714 | A | A | | | 2060.9052 | 2062.8 | 2061 | |
| I-715 | A | A | | | 2116.9678 | 2118.8 | 2116.9 | |
| I-716 | A | B | | | 2116.9678 | 2118.9 | 2116.9 | |
| I-717 | A | B | | | 2130.9835 | 2132.9 | 2131.2 | |
| I-718 | A | C | | | 2130.9835 | 2132.8 | 2131 | |
| I-719 | A | A | ++ | + | 2100.9365 | 2102.8 | 2100.4 | |
| I-720 | A | C | ++ | + | 2100.9365 | 2102.8 | 2100.7 | |
| I-721 | A | B | ++ | + | 2090.9158 | 2092.8 | 2090.9 | |
| I-722 | A | B | + | | 2150.9522 | 2152.9 | 2151 | |
| I-723 | A | B | | | 2117.9267 | 2119.7 | 2117.8 | |
| I-724 | A | B | | | 2131.9424 | 2133.8 | 2132.2 | |
| I-725 | A | C | | | 2189.9631 | 2191.9 | 2189.7 | |
| I-726 | B | C | | | 2189.9631 | 2191.9 | 2190.2 | |
| I-727 | A | B | | | 2166.9471 | 2168.9 | 2166.3 | |
| I-728 | A | B | | | 2166.9471 | 2169 | 2166.7 | |
| I-729 | A | A | | | 2122.9209 | 2124.7 | 2122.7 | |
| I-730 | A | | | | 2086.9209 | 2088.7 | 2086.7 | |
| I-731 | A | | | | 2128.9678 | 2130.8 | 2128.9 | |
| I-732 | A | A | | | 2134.9209 | 2136.7 | 2134.4 | |
| I-733 | A | A | | | 2098.9209 | 2100.8 | 2098.9 | |
| I-734 | A | B | | | 2140.9678 | 2142.8 | 2140.6 | |
| I-735 | A | C | | | 2148.9365 | 2150.8 | 2149.1 | |
| I-736 | A | A | | | 2112.9365 | 2114.8 | 2113 | |
| I-737 | A | A | | | 2154.9835 | 2156.8 | 2154.6 | |
| I-738 | A | C | | | 2232.9365 | | | |
| I-739 | A | B | | | 2232.9365 | 2234.8 | 2232.8 | |
| I-740 | A | A | | | 2196.9365 | 2199 | 2197 | |
| I-741 | E | D | | +++ | 2074.9209 | 2076.8 | 2075 | |
| I-742 | E | D | | +++ | 2074.9209 | 2076.8 | 2075 | |
| I-743 | C | D | | | 2101.8954 | 2103 | 2100.8 | |
| I-744 | A | D | | | 2115.9111 | 2117.9 | 2115.7 | |
| I-745 | A | C | | | 2115.9111 | 2117.9 | 2115.9 | |
| I-746 | A | C | | | 2130.9835 | 2132.8 | 2130.9 | |
| I-747 | A | B | | + | 2102.9522 | 2104.8 | 2103 | |
| I-748 | A | C | | + | 2138.927 | 2140.8 | 2138.6 | |
| I-749 | A | C | | | 2164.9678 | 2166.9 | 2164.9 | |
| I-750 | A | B | | | 2118.9471 | 2120.8 | 2118.3 | |
| I-751 | C | D | | | 2391.1975 | 2393.7 | 2391.6 | |
| I-752 | B | D | | | 2154.9271 | 1079.2 | 2155.4 | |
| I-753 | A | C | | + | 2136.9365 | 2138.9 | 2137 | |
| I-754 | A | C | | | 2110.8879 | 2112.9 | 2111.3 | |
| I-755 | A | C | | | 2186.9192 | 1046.2 | 1044.3 | |
| I-756 | A | D | | | 2186.9192 | 2188.9 | 2186.5 | |
| I-757 | A | | | | 2157.8749 | 2159.8 | 2157.7 | |
| I-758 | B | | | | 2157.8749 | 2159.9 | 2158.6 | |
| I-759 | B | | | | 2171.8905 | 2173.9 | 2171.4 | |
| I-760 | B | | | | 2171.8905 | 2173.9 | 2171.3 | |
| I-761 | A | | | | 2060.9458 | 2063.9 | 2062.4 | |
| I-762 | A | C | | | 2060.9458 | 2063.8 | 2061.8 | |
| I-763 | A | | | | 2021.055 | 2023.1 | 2021.4 | |
| I-764 | A | C | | | 2021.055 | 2023.1 | 2021.4 | |
| I-765 | B | | | | 2070.9342 | 2075 | 2072.9 | |
| I-766 | B | | | | 2070.9342 | 2073.9 | 2072.8 | |
| I-767 | A | | | | 2043.0394 | 2045.1 | 2043.5 | |
| I-768 | A | | | | 2043.0394 | 2045.2 | 2043.3 | |
| I-769 | A | | | | 2057.055 | 2059.2 | 2057.6 | |
| I-770 | A | C | | | 2057.055 | 2059.2 | 2057.1 | |
| I-771 | A | | | | 2063.0114 | 2065.1 | 2063.4 | |
| I-772 | A | C | | + | 2063.0114 | 2065.1 | 2063.3 | |
| I-773 | B | | | | 2063.0114 | 2065.2 | 2063.4 | |
| I-774 | C | D | | | 2063.0114 | 2065.1 | 2063.1 | |
| I-775 | A | C | | | 2033.055 | 2035.1 | 2033.5 | |
| I-776 | A | C | | | 2033.055 | 2035.2 | 2033.2 | |
| I-777 | B | | | | 2136.9519 | 2138.8 | 2136.9 | |
| I-778 | A | | | | 2150.9675 | 2152.8 | 2150.6 | |
| I-779 | B | | | | 2150.9675 | 2152.8 | 2151.1 | |
| I-780 | A | | | | 2074.9209 | 2076.8 | 2074.8 | |
| I-781 | A | D | | | 2088.9365 | 2090.8 | 2088.8 | |
| I-782 | A | B | | | 2216.9951 | 2219 | 2216.7 | |
| I-783 | A | C | | | 2359.0693 | 2361.1 | 2359.7 | |
| I-784 | A | | | | 2501.1436 | 1252.3 | 1250.6 | |
| I-785 | A | B | | + | 2203.9999 | 2205.9 | 2204.2 | |
| I-786 | A | C | | + | 2017.8994 | 2019.7 | 2017.6 | |
| I-787 | A | B | | | 2088.9365 | 2090.8 | 2089.2 | |
| I-788 | A | C | | +++ | 1975.8525 | 1977.7 | 1975.8 | |
| I-789 | A | C | | + | 2188.9638 | 2191 | 2188.8 | |
| I-790 | A | C | | + | 2331.038 | 2333.1 | 2331.5 | |
| I-791 | A | | | | 2175.9686 | 2178 | 2175.7 | |
| I-792 | A | D | | | 1975.8525 | 1977.7 | 1975.6 | |
| I-793 | A | D | | | 2046.8896 | 2048.9 | 2046.8 | |
| I-794 | A | | | | 2046.8896 | 2077 | 2075.8 | |
| I-795 | A | D | | +++ | 2188.9638 | 2191.1 | 2189.3 | |
| I-796 | A | | | | 2188.9638 | 2191 | 2189 | |
| I-797 | A | | | | 2175.9686 | 2178.2 | 2176 | |
| I-798 | A | | | | 2175.9686 | 2178.1 | 2176.2 | |
| I-799 | A | C | | | 2102.9522 | 2105 | 2103.4 | |
| I-800 | A | C | | | 2102.9522 | 2105 | 2103.1 | |
| I-801 | A | D | | | 2116.9678 | 2119 | 2117.1 | |
| I-802 | A | C | | | 2103.9474 | 2106 | 2104.2 | |
| I-803 | A | A | | | 2080.9586 | 2082.9 | 2081 | |
| I-804 | A | A | | | 2259.0421 | 2261.1 | 2259.4 | |
| I-805 | A | B | | + | 2259.0421 | 2261.1 | 2259 | |
| I-806 | A | B | | + | 2372.1261 | 2374.2 | 2371.9 | |
| I-807 | A | C | | | 2330.0792 | 2332.1 | 2330.3 | |
| I-808 | B | | | | 2443.1632 | 2445.3 | 2442.9 | |
| I-809 | B | | | | 2443.1632 | 2445.3 | 2443.4 | |
| I-810 | B | | | | 2556.2473 | 1280 | 1278.2 | |
| I-811 | B | | | | 2556.2473 | 1280 | 1278 | |
| I-812 | A | B | | + | 2613.2688 | 1308.4 | 1306.7 | |
| I-813 | A | B | | + | 2684.3059 | 1344 | 1342.1 | |
| I-814 | A | C | | + | 2625.3052 | 1314.4 | 1312.7 | |
| I-815 | A | C | | + | 2696.3423 | 1350 | 1348.4 | |
| I-816 | A | A | | + | 2611.2644 | 1307.4 | 1305.9 | |
| I-817 | A | B | | + | 2682.3015 | 1342.9 | 1341.3 | |
| I-818 | A | D | | | 2155.9424 | 2157.8 | 2155.4 | A |
| I-819 | B | | | | 2074.9209 | 2076.8 | 2074.9 | |
| I-820 | A | C | | | 2150.9675 | 2153.2 | 2151.2 | |
| I-821 | A | | | | 2150.9675 | 2152.9 | 2151.1 | |
| I-822 | A | B | | | 2237.9995 | 2240.2 | 2238.2 | |
| I-823 | A | C | | | 2134.8862 | 2137.9 | 2135.9 | |
| I-824 | A | | | | 2162.9175 | 2166 | 2164.7 | |
| I-825 | A | C | | | 2162.9175 | 2165.6 | 2163.9 | |
| I-826 | A | C | | | 2249.9495 | 2252.8 | 2250.7 | |
| I-827 | A | C | | | 2176.9331 | 2180.1 | 2178.8 | |
| I-828 | B | | | | 2176.9331 | 1090.6 | 1088.7 | |
| I-829 | B | | | | 2263.9652 | 2267.1 | 2265.4 | |
| I-830 | B | | | | 2263.9652 | 1134.1 | 1132.5 | |
| I-831 | A | C | | | 2066.8988 | 2069.8 | 2068.2 | |
| I-832 | A | C | | | 2066.8988 | 1035.5 | 1034.1 | |
| I-833 | A | C | | | 2094.9301 | 2097.8 | 2096.2 | |
| I-834 | A | B | | | 2094.9301 | 1049.5 | 1047.1 | |
| I-835 | A | C | | | 2181.9621 | 2184.9 | 2183.1 | |
| I-836 | A | C | | | 2181.9621 | 1093.2 | 1090.8 | |
| I-837 | A | C | | | 2108.9458 | 2112 | 2109.4 | |
| I-838 | A | C | | | 2108.9458 | 2111.8 | 2109.3 | |
| I-839 | A | B | | | 2195.9778 | 2198.9 | 2197.2 | |
| I-840 | A | C | | | 2195.9778 | 2198.9 | | |
| I-841 | A | B | | | 2162.9522 | 2165.3 | 2163.4 | |
| I-842 | A | B | | | 2122.9209 | 2125.2 | 2123.2 | |
| I-843 | A | B | | | 2134.9209 | 2137.2 | 2135 | |
| I-844 | A | B | | | 2160.9365 | 2163.2 | 2160.9 | |
| I-845 | A | C | | | 2244.9365 | 1124.3 | 1122.7 | |
| I-846 | A | C | | | 2174.9522 | 2177.3 | 2175.9 | |
| I-847 | A | C | | | 2146.9209 | 2149.2 | 2147.1 | |
| I-848 | A | C | | | 2134.9209 | 2137.2 | 2135.3 | |
| I-849 | A | | | | 2100.9365 | 2102.9 | 2101.2 | |
| I-850 | A | B | | | 2126.9522 | 2129.1 | 2127.1 | |
| I-851 | A | | | | 2210.9522 | 2213.2 | 2210.8 | |
| I-852 | A | B | | | 2140.9678 | 2143.3 | 2141.1 | |
| I-853 | A | | | | 2140.9678 | 2142.9 | 2140.9 | |
| I-854 | A | | | | 2112.9365 | 2115.2 | 2112.9 | |
| I-855 | A | | | | 2100.9365 | 2102.9 | 2101.1 | |
| I-856 | A | | | | 2072.9052 | 2074.9 | 2073.2 | |
| I-857 | A | | | | 2098.9209 | 2101.2 | 2098.8 | |
| I-858 | A | | | | 2182.9209 | 1093.3 | 1091.3 | |
| I-859 | A | B | | | 2112.9365 | 2115.1 | 2112.9 | |
| I-860 | A | C | | | 2084.9052 | 2086.9 | 2084.9 | |
| I-861 | A | C | | | 2072.9052 | 1038.2 | 1036.8 | |
| I-862 | A | B | | | 2077.8647 | 2080.7 | 2078.9 | |
| I-863 | A | | | | 2077.8647 | 2080.8 | 2079.5 | |
| I-864 | A | C | | | 2174.9175 | 1089.7 | 1087 | |
| I-865 | A | C | | | 2164.8968 | 1084.6 | 1082.6 | |
| I-866 | B | | | | 2224.9331 | 2228.3 | 2226.2 | |
| I-867 | A | A | | + | 2015.8338 | 1010.2 | 1008 | |
| I-868 | A | C | | + | 2112.8865 | 2116.2 | 2113.3 | |
| I-869 | A | B | | + | 2102.8658 | 2105.9 | 2103.4 | |
| I-870 | A | C | | | 2162.9022 | 2166.4 | 2164 | |
| I-871 | A | A | | + | 2009.8773 | 2013.5 | 2010.8 | |
| I-872 | A | C | | + | 2106.9301 | 2110 | 2108.5 | |
| I-873 | A | B | | | 2096.9094 | 2099.9 | 2098.3 | |
| I-874 | A | C | | | 2156.9458 | 2159.8 | 2158.3 | |
| I-875 | A | | | | 2038.8821 | 2041 | 2039.2 | |
| I-876 | A | | | | 2109.9192 | 2112.1 | 2110.2 | |
| I-877 | A | | | | 2238.9982 | 2241.2 | 2239.3 | |
| I-878 | A | | | | 2032.9256 | 2035 | 2033.1 | |
| I-879 | A | | | | 2103.9628 | 1053.8 | 1052 | |
| I-880 | A | | | | 2233.0417 | 2235.2 | 2233.5 | |
| I-881 | A | | | | 2050.832 | 2053.8 | 2052.9 | |
| I-882 | A | | | | 2121.8692 | 2124.8 | 2123.3 | |
| I-883 | A | | | | 2250.9481 | 2254 | 2252.6 | |
| I-884 | A | | | | 2044.8756 | 1024.6 | 1023 | |
| I-885 | A | | | | 2115.9127 | 2118.8 | 2117.4 | |
| I-886 | A | | | | 2244.9917 | 2247.9 | 2246.1 | |
| I-887 | A | | | | 2100.913 | 2103 | 2101.1 | |
| I-888 | B | | | | 2301.0291 | 1152.4 | 1150.4 | |
| I-889 | B | | | | 2301.0291 | 1152.4 | 1150.6 | |
| I-890 | A | | | | 2112.863 | 2115.8 | 2113.9 | |
| I-891 | A | C | | | 2112.863 | 1058.7 | 1057 | |
| I-892 | A | D | | | 2183.9001 | 1094.2 | 1091.8 | |
| I-893 | B | | | | 2312.9791 | 1158.8 | 1156.7 | |
| I-894 | A | C | | | 2018.91 | 2021 | 2019.3 | |
| I-895 | A | | | | 2018.91 | 2021.1 | 2019.3 | |
| I-896 | A | C | | | 2089.9471 | 2092.1 | 2090.2 | |
| I-897 | A | | | | 2089.9471 | 2092.1 | 2090.2 | |
| I-898 | A | C | | | 2219.0261 | 2221.3 | 2219.3 | |
| I-899 | A | | | | 2219.0261 | 1111.5 | 1109.5 | |
| I-900 | A | D | | | 2094.9413 | 2097.1 | 2095.2 | |
| I-901 | A | D | | | 2165.9784 | 2168.2 | 2166.3 | |
| I-902 | B | | | | 2295.0574 | 1149.4 | 1147.6 | |
| I-903 | A | D | | ++ | 2178.9332 | 1091.4 | 1089.5 | |
| I-904 | A | C | | | 2177.9379 | 2180.2 | 2178.1 | |
| I-905 | A | | | | 2177.9379 | 1090.8 | 1089 | |
| I-906 | A | | | | 2176.9427 | 1090.4 | 1087.9 | |
| I-907 | A | | | | 2138.927 | 2141.2 | 2139.4 | |
| I-908 | A | B | | + | 2179.9787 | 1091.9 | 1089.8 | |
| I-909 | A | C | | | 2138.927 | 2141.2 | 2139.4 | |
| I-910 | A | | | | 2138.927 | 2141.2 | 2139.4 | |
| I-911 | A | | | | 2138.927 | 2141.2 | 2139 | |
| I-912 | A | | | | 2138.927 | 2141.4 | 2139.7 | |
| I-913 | A | B | | | 2086.8709 | 2090.1 | 2089.3 | |
| I-914 | A | C | | | 2086.8709 | 1045.8 | 1044.2 | |
| I-915 | A | C | | | 2215.9499 | 2219 | 2217.6 | |
| I-916 | A | C | | | 2215.9499 | 1110.3 | 1108.6 | |
| I-917 | A | C | | | 2114.9022 | 2118 | 2116.6 | |
| I-918 | A | C | | | 2114.9022 | 2118.2 | 2116.3 | |
| I-919 | A | C | | | 2148.8865 | 2151.2 | 2150.3 | |
| I-920 | A | C | | | 2148.8865 | 2152 | 2150.1 | |
| I-921 | B | C | | | 2092.8273 | 2095.6 | 2093.9 | |
| I-922 | A | C | | | 2092.8273 | 2095.6 | 2093.9 | |
| I-923 | A | B | | | 2003.8838 | 2005.7 | 2003.5 | |
| I-924 | A | C | | | 2003.8838 | 2006.2 | 2004.4 | |
| I-925 | A | B | | | 2203.9999 | 2205.9 | 2204.1 | |
| I-926 | A | C | | | 2102.9522 | 2105.3 | 2103.4 | |
| I-927 | A | B | | | 2136.9365 | 2138.9 | 2136.9 | |
| I-928 | E | | | | 2106.9624 | 1055.3 | 1053.6 | |
| I-929 | C | | | | 2087.9525 | 2090.1 | 2088.1 | |
| I-930 | C | | | | 2087.9525 | 1045.8 | 2088.1 | |
| I-931 | E | | | | 2112.9188 | 1057.7 | 2112.5 | |
| I-932 | E | | | | 2036.9052 | 2039.1 | 2037.1 | A |
| I-933 | E | | | | 2036.8147 | 2039 | 2037.2 | |
| I-934 | E | | | | 2040.846 | 2042.7 | 2041 | A |
| I-935 | B | D | | | 2088.9365 | 2091.3 | 2089.2 | |
| I-936 | B | D | | | 2063.0114 | 2065.2 | 2063.2 | |
| I-937 | B | D | | | 2063.0114 | 2065.3 | 2063.2 | |
| I-938 | B | | | | 2077.0271 | 2079.3 | 2077.8 | |
| I-939 | C | | | | 2077.0271 | 1040.4 | 2077.2 | |
| I-940 | A | C | | | 2124.9365 | 2127.4 | 2125.3 | |
| I-941 | A | | | | 2124.9365 | 2127.3 | 2125.2 | |
| I-942 | A | D | | | 2074.9614 | 2077.9 | 2075.2 | |
| I-943 | B | | | | 2088.9771 | 1046.8 | 1044.9 | |
| I-944 | A | D | | | 2088.9771 | 1046.8 | 2090.1 | |
| I-945 | A | C | | | 2136.8865 | 2140.4 | 2138.5 | |
| I-946 | A | | | | 2136.9519 | 2139.2 | 2137.2 | |
| I-947 | A | D | | | 2060.9052 | 2063.3 | 2061.9 | |
| I-948 | A | D | | | 2074.9209 | 2077.2 | 2076.4 | |
| I-949 | B | | | | 2144.9958 | 2147 | 2145.1 | |
| I-950 | B | | | | 2156.9458 | 2159.7 | 2157.6 | |
| I-951 | B | | | | 2156.9458 | 2159.8 | 2157.4 | |
| I-952 | C | | | | 2274.0748 | 2276.2 | 2274.3 | |
| I-953 | C | | | | 2274.0748 | 2276.2 | 2274 | |
| I-954 | C | | | | 2286.0247 | 2289 | 2288.2 | |
| I-955 | B | B | | | 2178.0748 | 1091 | 2178.7 | |
| I-956 | A | | | | 2190.0247 | 1097.3 | 2191.4 | |
| I-957 | A | C | | | 2074.9209 | 1039.3 | 1037.6 | |
| I-958 | E | | | | 2088.9365 | 1046.3 | 1044.4 | |
| I-959 | E | | | | 2088.9365 | 1046.3 | 1044.4 | |
| I-960 | E | | | | 2088.9365 | 1046.3 | 1044.5 | |
| I-961 | A | D | | | 2088.9365 | 2091.1 | 2089.1 | |
| I-962 | E | | | | 2088.9365 | 2191.2 | 2089.2 | |
| I-963 | B | | | | 2078.9522 | 1041.3 | 1039.4 | A |
| I-964 | E | | | | 2092.9678 | 2095.1 | 2093.2 | A |
| I-965 | C | | | | 2092.9678 | 2095.2 | 2093.8 | A |
| I-966 | E | | | | 2092.9678 | 2095.3 | 2093.2 | A |
| I-967 | B | | | | 2092.9678 | 2095.2 | 2092.9 | |
| I-968 | E | | | | 2091.9838 | 2094.2 | 2091.7 | |
| I-969 | A | A | | | 2046.8896 | | | |
| I-970 | A | B | | + | 2046.8896 | | | |
| I-971 | A | B | | | 2074.9209 | | | |
| I-972 | A | | | | 2088.9365 | | | |
| I-973 | A | | | | 2088.9365 | | | |
| I-974 | A | | | | 2088.9365 | | | |
| I-975 | A | | | | 2116.9678 | | | |
| I-976 | A | | | | 2072.9052 | | | |
| I-977 | A | | | | 2072.9052 | | | |
| I-978 | A | | | | 2100.9365 | | | |
| I-979 | A | | | | 2114.9522 | | | |
| I-980 | A | | | | 2142.9835 | | | |
| I-981 | A | | | | 2142.9835 | | | |
| I-982 | A | B | | + | 2156.9052 | 2159.2 | 2157.5 | |
| I-983 | A | B | | | 2156.9052 | 2159.3 | 2157.2 | |
| I-984 | A | B | | + | 2184.9365 | 1094.4 | 2185.9 | |
| I-985 | A | A | | | 2198.9522 | 1101.4 | 1099.6 | |
| I-986 | A | A | | + | 2198.9522 | 2201.3 | 2198.9 | |
| I-987 | A | B | | + | 2058.8896 | 2061.1 | 2059.3 | |
| I-988 | A | B | | + | 2086.9209 | 2089.2 | 2087.6 | |
| I-989 | A | C | | + | 2100.9365 | 2103.2 | 2101.2 | |
| I-990 | A | C | | | 2100.9365 | 2103.2 | 2101.4 | |
| I-991 | A | | | +++ | 2128.9678 | 2131.3 | 2129.2 | |
| I-992 | E | | | | 2032.8739 | 2035.2 | 2033.2 | |
| I-993 | D | | | | 2008.8739 | 2011.1 | 2009.5 | |
| I-994 | A | | | | 2058.8896 | 2061.3 | 2059 | |
| I-995 | A | | | | 2058.8896 | 2061.2 | 2059.3 | |
| I-996 | A | B | | | 2072.9052 | 2075.2 | 2073.3 | |
| I-997 | A | | | | 2116.9315 | 2119.4 | 2116.8 | |
| I-998 | A | C | | | 2116.9315 | 2119.4 | 2116.8 | |
| I-999 | A | C | | | 2072.9052 | 2075.4 | 2073.7 | |
| I-1000 | A | | | | 2142.8896 | 2145.3 | 2142.6 | |
| I-1001 | A | | | | 2142.8896 | 2145.3 | 2142.4 | |
| I-1002 | A | | | | 2156.9052 | 2159.2 | 2157.3 | |
| I-1003 | A | | | | 2156.9052 | 2159.3 | 2157.4 | |
| I-1004 | A | C | | | 2186.9158 | 2189.3 | 2187 | |
| I-1005 | A | | | | 2200.9315 | 2203.4 | 2201 | |
| I-1006 | A | D | | | 2200.9315 | 2203.4 | 2200.9 | |
| I-1007 | A | | | | 2200.9315 | 2203.4 | 2201.2 | |
| I-1008 | A | C | | | 2156.9052 | 2159.4 | 2156.9 | |
| I-1009 | A | C | | | 2156.9052 | 2159.3 | 2157.4 | |
| I-1010 | A | B | | | 2072.8552 | 2076.9 | 2075.3 | |
| I-1011 | A | | | | 2116.9315 | 2119.2 | 2116.6 | |
| I-1012 | A | B | | | 2152.9315 | 2155.3 | 2153 | |
| I-1013 | A | C | | | 2104.9315 | 2107.2 | 2105.5 | |
| I-1014 | A | C | | | 2086.9209 | 2089.2 | 2087.6 | |
| I-1015 | A | C | | | 2122.9209 | 2125.3 | 2123.2 | |
| I-1016 | A | D | | | 2074.9209 | 2077.2 | 2075.1 | |
| I-1017 | A | D | | | 2142.9835 | 2145.5 | 2143.2 | |
| I-1018 | A | C | | | 2178.9835 | 2181.4 | 2178.9 | |
| I-1019 | B | D | | | 2130.9835 | 2133.4 | 2131.9 | |
| I-1020 | A | C | | | 2130.9471 | 2133.3 | 2131.7 | |
| I-1021 | B | D | | | 2118.9471 | 2121.3 | 2119.3 | |
| I-1022 | B | | | | | | | A |
| I-1023 | A | D | | | 2060.9052 | 2061.9 | 2061.9 | |
| I-1024 | E | | | | 2043.8651 | 2047.4 | 2045.4 | |
| I-1025 | F | | | | 2208.0100 | 2210.6 | 2208.4 | |
| I-1026 | A | D | | | 2157.9580 | 2160.5 | 2158.5 | |
| I-1027 | A | D | | +++ | 2157.9580 | 2060.5 | 2158.0 | |
| I-1028 | A | D | | | 2219.9737 | 2222.7 | 2220.6 | |
| I-1029 | A | | | | 2219.9737 | 2222.6 | 2220.8 | |
| I-1030 | A | | | | 2200.0050 | 2202.7 | 2201.0 | |
| I-1031 | B | | | | 2143.9424 | 1074.0 | 1072.0 | |
| I-1032 | B | | | | 2205.9580 | 1105.0 | 1102.9 | |
| I-1033 | B | | | | 2185.9893 | 1095.0 | 1092.9 | |
| I-1034 | A | | | | 2143.9424 | 2146.4 | 2144.9 | |
| I-1035 | A | | | | 2205.9580 | 1105.1 | 1103.3 | |
| I-1036 | A | | | | 2185.9893 | 2188.6 | 2185.9 | |
| I-1037 | A | | | + | 2027.8338 | 2031.1 | 2029.4 | |
| I-1038 | A | | | + | 2041.8494 | 2044.9 | 2043.6 | |
| I-1039 | A | | | | 2041.8494 | 2044.9 | 2043.6 | |
| I-1040 | A | | | + | 2027.8338 | 2031.0 | 2029.4 | |
| I-1041 | A | | | | 2027.8338 | 2031.1 | 2029.3 | |
| I-1042 | A | | | + | 2041.8494 | 2045.2 | 2042.2 | |
| I-1043 | A | | | | 2041.8494 | 2045.1 | 2043.3 | |
| I-1044 | A | | | + | 2053.8494 | 2056.9 | 2054.4 | |
| I-1045 | A | | | | 2053.8494 | 2057.1 | 2054.9 | |
| I-1046 | A | | | | 2067.8651 | 2071.4 | 2069.4 | |
| I-1047 | A | | | + | 2067.8651 | 2071.2 | 2068.5 | |
| I-1048 | A | | | + | 2021.8773 | 2025.2 | 2023.2 | |
| I-1049 | A | | | | 2021.8773 | 2025.4 | 2024.6 | |
| I-1050 | A | | | + | 2035.8930 | 2039.0 | 2037.3 | |
| I-1051 | A | | | | 2035.8930 | 2039.1 | 2036.8 | |
| I-1052 | A | | | + | 2021.8773 | 2024.9 | 2022.7 | |
| I-1053 | A | | | | 2021.8773 | 2025.0 | 2023.7 | |
| I-1054 | A | | | + | 2035.8930 | 2039.1 | 2037.6 | |
| I-1055 | A | | | | 2035.8930 | 2039.6 | 2037.4 | |
| I-1056 | A | | | + | 2047.8930 | 2051.0 | 2049.1 | |
| I-1057 | A | | | | 2047.8930 | 2051.5 | 2049.1 | |
| I-1058 | A | | | ND | 2061.9086 | 2065.1 | 2063.0 | |
| I-1059 | A | | | | 2061.9086 | 2065.0 | 2063.2 | |
| I-1060 | A | | | | 1947.8617 | 1951.0 | 1948.2 | |
| I-1061 | A | | | | 1961.8773 | 1964.9 | 1963.5 | |
| I-1062 | A | | | | 2140.9427 | 2143.5 | 2141.2 | |
| I-1063 | A | | | | 2187.9286 | 2190.2 | 2188.8 | |
| I-1064 | A | | | +++ | 2190.9583 | 2193.6 | 2190.9 | |
| I-1065 | A | | | | 2155.9536 | 2158.5 | 2156.9 | |
| I-1066 | A | | | | 2201.9631 | 2204.8 | 2202.1 | |
| I-1067 | A | D | | | 2151.9474 | 2154.7 | 2152.7 | |
| I-1068 | A | | | | 2141.9379 | 1073.0 | 1070.8 | |
| I-1069 | A | | | | 2140.9427 | 2143.5 | 2141.6 | |
| I-1070 | A | | | | 2165.9631 | 2168.8 | 2167.7 | |
| I-1071 | A | | | +++ | 2165.9631 | 1082.2 | 1093.4 | |
| I-1072 | A | | | | 2165.9631 | 2168.7 | 2167.0 | |
| I-1073 | A | | | | 2152.9427 | 2155.6 | 2154.1 | |
| I-1074 | A | | | | 2157.9580 | 2160.3 | 2158.4 | |
| I-1075 | A | | | +++ | 2141.9379 | 2144.3 | 2142.2 | |
| I-1076 | A | D | | | 2190.9583 | 2193.8 | 2191.4 | |
| I-1077 | A | | | | 2190.9583 | 2193.6 | 2191.6 | |
| I-1078 | A | | | +++ | 2117.9631 | 2120.7 | 2118.1 | |
| I-1079 | A | | | +++ | 2187.0209 | 2190.0 | 2187.8 | |
| I-1080 | A | | | | 2187.0209 | 1095.6 | 1093.4 | |
| I-1081 | A | | | +++ | 2173.0053 | 2175.6 | 2173.5 | |
| I-1082 | A | | | | 2173.0053 | 2175.8 | 2173.8 | |
| I-1083 | A | | | | 2176.9097 | 1090.6 | 1088.6 | |
| I-1084 | A | | | | 2176.9097 | 1090.6 | 1088.9 | |
| I-1085 | A | | | | 2223.9144 | 2226.5 | 2224.2 | |
| I-1086 | A | | | | 2025.8293 | 2029.1 | | |
| I-1087 | A | | | | 2025.8293 | 2029.0 | 2027.8 | |
| I-1088 | A | | | | 2096.8665 | 2100.2 | | |
| I-1089 | A | | | | 2011.8137 | 2015.0 | 2012.6 | |
| I-1090 | A | | | | 2073.8293 | 2077.1 | 2074.9 | |
| I-1091 | A | | | | 2144.8665 | 2148.2 | | |
| I-1092 | A | | | | 2019.8729 | 2023.1 | 2020.6 | |
| I-1093 | A | | | | 2090.9100 | 2094.7 | 2092.3 | |
| I-1094 | A | | | | 2005.8573 | 2009.1 | | |
| I-1095 | A | | | | 2076.8944 | 2080.1 | 2078.2 | |
| I-1096 | A | | | | 2067.8729 | 2071.2 | 2069.4 | |
| I-1097 | A | | | | 2138.9100 | 2142.1 | 2140.5 | |
| I-1098 | A | | | | 1975.9430 | 1978.2 | 1976.6 | |
| I-1099 | A | | | | 2032.9645 | 2035.3 | 2033.8 | |
| I-1100 | A | | | | 1961.9274 | 1964.2 | 1962.8 | |
| I-1101 | A | | | | 2094.9801 | 2097.3 | 2095.4 | |
| I-1102 | A | | | + | 2023.9430 | 2026.3 | 2024.9 | |
| I-1103 | A | | | + | 1969.9866 | 1972.3 | 1970.1 | |
| I-1104 | A | | | | 2027.0081 | 2029.3 | 2027.7 | |
| I-1105 | A | | | | 1955.9709 | 1958.2 | 1956.5 | |
| I-1106 | A | | | + | 2089.0237 | 2091.4 | 2089.3 | |
| I-1107 | A | | | + | 2017.9866 | 2020.3 | 2018.6 | |
| I-1108 | A | | | + | 2111.8338 | 2115.3 | 2113.8 | |
| I-1109 | A | | | | 2182.8709 | 2186.3 | 2185.1 | |
| I-1110 | A | | | | 2198.8658 | 2202.1 | 2200.3 | |
| I-1111 | A | | | + | 2097.8181 | 2101.6 | 2099.3 | |
| I-1112 | A | | | | 2168.8552 | 2172.1 | 2170.3 | |
| I-1113 | A | | | | 2184.8501 | 2188.3 | 2185.9 | |
| I-1114 | A | | | | 2173.8494 | 2178.2 | 2176.5 | |
| I-1115 | A | | | | 2244.8865 | 2247.5 | | |
| I-1116 | A | | | | 2260.8814 | 1032.9 | 1030.7 | |
| I-1117 | A | | | + | 2137.8494 | 1071.3 | 2139.3 | |
| I-1118 | A | | | + | 2208.8865 | 1106.8 | 2210.1 | |
| I-1119 | A | | | | 2224.8814 | 2228.1 | 2226.4 | |
| I-1120 | A | | | | 2126.9634 | 2129.4 | 2127.3 | |
| I-1121 | A | | | | 2126.9634 | 2129.4 | 2127.4 | |
| I-1122 | B | | | | 2098.9321 | 2103.4 | 2101.6 | |
| I-1123 | B | | | | 2098.9321 | 2103.4 | 2101.8 | |
| I-1124 | A | | | | 2110.9685 | 2113.4 | | |
| I-1125 | A | | | | 2082.9372 | 2085.3 | 2083.4 | |
| I-1126 | A | | | | 2111.9274 | 2114.3 | 2111.9 | |
| I-1127 | A | | | | 2111.9274 | 2114.3 | 2112.1 | |
| I-1128 | A | D | | | 2163.9686 | 2166.4 | 2164.3 | |
| I-1129 | A | D | | | 2163.9686 | 2166.4 | 2164.6 | |
| I-1130 | E | D | | | 2176.0050 | 2155.9 | 2154.0 | |
| I-1131 | E | D | | | 2176.0050 | 2114.0 | 2112.7 | |
| I-1132 | A | D | | | 2190.0206 | 2192.6 | 2190.7 | |
| I-1133 | A | D | +++ | | 2190.0206 | 2192.5 | 2190.6 | |
| I-1134 | A | D | +++ | | 2177.9842 | 2180.5 | 2178.6 | |
| I-1135 | A | D | | | 2177.9842 | 2180.5 | 2179.0 | |
| I-1136 | A | D | | | 2224.0050 | 2226.5 | 2224.7 | |
| I-1137 | B | D | | | 2224.0050 | 2226.6 | 2224.9 | |
| I-1138 | A | D | | | 2245.0264 | 2247.5 | 2245.3 | |
| I-1139 | A | D | | | 2245.0264 | 2247.6 | 2245.8 | |
| I-1140 | A | D | | | 2235.0057 | 2237.5 | 2235.2 | |
| I-1141 | A | D | | | 2235.0057 | 1119.6 | 1117.7 | |
| I-1142 | A | D | | | 2219.0108 | 2221.5 | 2219.9 | |
| I-1143 | A | D | | | 2219.0108 | 2221.5 | 2219.4 | |
| I-1144 | A | D | | +++ | 2167.9999 | 2170.5 | 2168.6 | A |
| I-1145 | A | D | ++ | + | 2180.0363 | 2180.6 | 2178.6 | A |
| I-1146 | A | D | | + | 2194.0519 | 2196.4 | 2194.1 | A |
| I-1147 | A | D | | +++ | 2182.0155 | 2184.5 | 2182.4 | A |
| I-1148 | A | D | | | 2228.0363 | 2230.4 | 2228.5 | A |
| I-1149 | B | D | | +++ | 2249.0577 | 2251.4 | 2249.6 | A |
| I-1150 | A | D | | +++ | 2239.0370 | 2241.5 | 2239.0 | A |
| I-1151 | A | D | | +++ | 2223.0421 | 2225.3 | 2223.4 | A |
| I-1152 | | | | | 2058.9624 | 2061.4 | 2059.2 | |
| I-1153 | E | | | | 2086.9937 | 2089.2 | 2087.3 | |
| I-1154 | E | | | | 2162.9344 | 2165.5 | 2163.2 | |
| I-1155 | A | | | + | 2586.2076 | 1295.1 | 1293.5 | |
| I-1156 | A | | | + | 2671.2716 | 1337.6 | 1336.2 | |
| I-1157 | A | | | + | 2671.2716 | 1337.7 | 1335.9 | |
| I-1158 | A | | | + | 2615.2593 | 1309.7 | 1307.7 | |
| I-1159 | A | | | | 2598.1963 | 1301.0 | 1299.2 | |
| I-1160 | A | | | | 2669.2335 | 1336.6 | 1334.5 | |
| I-1161 | B | | | | 2123.0114 | 2125.4 | 2123.0 | |
| I-1162 | A | | | +++ | 2002.8998 | 2005.3 | 2003.7 | |
| I-1163 | A | | | +++ | 2098.8998 | 2101.4 | | |
| I-1164 | A | | | +++ | 2169.9369 | 2172.5 | 2170.7 | |
| I-1165 | A | | | +++ | 1988.8841 | 1991.3 | 1989.3 | |
| I-1166 | A | | | +++ | 1988.8841 | 1991.3 | 1989.6 | |
| I-1167 | A | | | +++ | 2059.9212 | 2062.3 | 2060.0 | |
| I-1168 | A | | | +++ | 2059.9212 | 2062.4 | 2060.4 | |
| I-1169 | A | | | +++ | 2075.9161 | 2078.3 | 2076.3 | |
| I-1170 | A | | | +++ | 2075.9161 | 2078.4 | 2076.5 | |
| I-1171 | A | | | +++ | 2008.8933 | 2012.0 | | |
| I-1172 | A | | | +++ | 2008.8933 | 2012.0 | 2009.9 | |
| I-1173 | A | | | +++ | 2079.9304 | 2083.1 | 2081.2 | |
| I-1174 | A | | | +++ | 2079.9304 | 2083.1 | 2081.4 | |
| I-1175 | A | | | +++ | 2104.8933 | 1054.7 | | |
| I-1176 | A | | | | 2104.8933 | 2108.1 | 2106.7 | |
| I-1177 | A | | | +++ | 2175.9304 | 1090.3 | 2176.1 | |
| I-1178 | A | | | +++ | 1994.8777 | 1998.0 | 1996.3 | |
| I-1179 | A | | | | 1994.8777 | 1998.0 | 1996.3 | |
| I-1180 | A | | | +++ | 2065.9148 | 2069.1 | 2066.7 | |
| I-1181 | A | | | +++ | 2350.0942 | 2352.7 | 2351.1 | |
| I-1182 | A | | | +++ | 2526.1990 | 1265.1 | 1263.5 | |
| I-1183 | A | | | + | 2878.4087 | 1441.3 | 1439.5 | |
| I-1184 | A | | | +++ | 2251.0258 | 2253.6 | 2251.6 | |
| I-1185 | A | | | +++ | 2427.1306 | 2429.8 | 2427.8 | |
| I-1186 | A | | | +++ | 2779.3403 | 1391.8 | 1389.8 | |
| I-1187 | A | | | +++ | 3131.5500 | 1568.0 | 1566.2 | |
| I-1188 | B | | | | 2074.9380 | 1039.3 | 1037.7 | |
| I-1189 | B | | | | 2074.9380 | 1039.2 | 1037.2 | |
| I-1190 | B | | | | 2074.9380 | 2077.1 | 2075.7 | |
| I-1191 | C | | | | 2161.9893 | 2164.4 | | |
| I-1192 | C | | | | 2161.9893 | 2164.4 | 2162.1 | |
| I-1193 | C | | | | 2146.9896 | 2149.6 | 2147.7 | |
| I-1194 | B | | | | 2146.9896 | 2149.1 | 2147.5 | |
| I-1195 | C | | | | 2119.9787 | 2122.4 | 2120.6 | |
| I-1196 | C | | | | 2119.9787 | 2122.3 | 2120.4 | |
| I-1197 | B | | | | 2133.9944 | 2136.4 | | |
| I-1198 | E | | | | 2133.9944 | 2136.4 | 2134.6 | |
| I-1199 | B | | | | 2161.9893 | 2164.4 | 2162.2 | |
| I-1200 | C | | | | 2161.9893 | 2164.5 | 2162.3 | |
| I-1201 | C | | | | 2146.9896 | 2149.4 | 2147.3 | |
| I-1202 | C | | | | 2146.9896 | 2149.4 | 2147.3 | |
| I-1203 | C | | | | 2119.9787 | 2122.3 | 2120.4 | |
| I-1204 | E | | | | 2119.9787 | 2122.4 | 2120.3 | |
| I-1205 | E | | | | 2133.9944 | 2136.4 | 2134.6 | |
| I-1206 | E | | | | 2133.9944 | 2136.4 | 2134.0 | |
| I-1207 | E | | | | 2176.0050 | 2178.4 | 2176.3 | |
| I-1208 | E | | | | 2176.0050 | 2178.4 | 2176.6 | |
| I-1209 | E | | | | 2219.9948 | 2222.4 | 2220.6 | |
| I-1210 | D | | | | 2219.9948 | 2222.4 | 2220.3 | |
| I-1211 | A | | | | 2219.9948 | 2222.5 | 2221.1 | |
| I-1212 | A | | | | 2219.9948 | 2223.3 | 2221.3 | |
| I-1213 | B | D | | | 2064.9729 | 2067.3 | 2065.6 | A |
| I-1214 | B | D | | | 2107.0199 | 2109.3 | 2107.3 | A |
| I-1215 | A | D | | | 2064.9729 | 2067.4 | 2064.8 | A |
| I-1216 | B | D | | | 2078.9886 | 2181.4 | 2179.3 | A |
| I-1217 | C | | | +++ | 2166.0206 | 2168.5 | 2166.2 | A |
| I-1218 | B | | | | 2151.0209 | 2153.6 | 2151.5 | A |
| I-1219 | E | | | | 2124.0100 | 2126.6 | 2124.7 | A |
| I-1220 | C | | | | 2138.0257 | 2140.4 | 2137.9 | A |
| I-1221 | D | | | +++ | 2166.0206 | 2168.5 | 2166.9 | A |
| I-1222 | C | | | | 2151.0209 | 2153.4 | 2151.3 | A |
| I-1223 | E | | | | 2124.0100 | 2126.4 | 2124.4 | A |
| I-1224 | E | | | | 2138.0257 | 2140.4 | 2138.5 | A |
| I-1225 | E | | | | 2180.0363 | 2182.4 | 2180.2 | A |
| I-1226 | D | | | | 2224.0261 | 2226.5 | 2224.4 | A |
| I-1227 | B | | | | 2224.0261 | 2226.5 | | A |
| I-1228 | E | | | | 2238.0417 | 2240.8 | 2238.9 | A |
| I-1229 | A | | | | 2157.9985 | 2161.2 | 2158.8 | A |
| I-1230 | A | | | + | 2086.9614 | 2090.4 | 2088.6 | A |
| I-1231 | A | | | + | 2163.9549 | 2167.1 | | A |
| I-1232 | A | | | + | 2092.9178 | 2096.0 | 2094.3 | A |
| I-1233 | A | | | | 2143.9829 | 2147.1 | 2145.4 | A |
| I-1234 | A | | | | 2072.9458 | 1038.8 | 1037.1 | A |
| I-1235 | A | | | | 2101.9359 | 1053.3 | 1051.3 | A |
| I-1236 | A | | | | 2030.8988 | 2033.9 | 2032.4 | A |
| I-1237 | A | | | | 2177.9672 | 2181.2 | 2180.2 | A |
| I-1238 | A | | | | 2106.9301 | 2110.4 | 2108.2 | A |
| I-1239 | A | | | | 2129.9672 | 2133.0 | 2131.5 | A |
| I-1240 | B | | | | 2058.9301 | 1031.7 | 2060.5 | A |
| I-1241 | A | | | | 2130.9413 | 1067.8 | 1066.0 | |
| I-1242 | A | | | | 1997.9274 | 2000.1 | 1998.1 | |
| I-1243 | A | | | | 1983.9117 | 1986.1 | 1984.5 | |
| I-1244 | A | | | | 2054.9488 | 2057.2 | 2055.1 | |
| I-1245 | A | | | | 2015.9179 | 2018.2 | 2016.2 | |
| I-1246 | A | | | | 2015.9179 | 2018.2 | 2016.8 | |
| I-1247 | A | | | | 2086.9551 | 2089.2 | 2087.1 | |
| I-1248 | A | | | | 2031.8884 | 2034.6 | 2032.3 | |
| I-1249 | A | | | | 2102.9255 | 2105.8 | 2104.4 | |
| I-1250 | A | | | | 2011.9430 | 2085.3 | 2083.3 | |
| I-1251 | A | | | | 2082.9801 | 2014.2 | 2012.6 | |
| I-1252 | A | | | | 2295.0098 | 2298.3 | 2296.0 | |
| I-1253 | A | | | | 2295.0098 | 2298.3 | 2295.8 | |
| I-1254 | A | | | | 2337.0568 | 1170.9 | 1169.2 | |
| I-1255 | A | | | | 2337.0568 | 2340.4 | 2338.6 | |
| I-1256 | A | | | | 2267.0149 | 1135.8 | 2268.1 | |
| I-1257 | A | | | | 2267.0149 | 1135.9 | 1134.3 | |
| I-1258 | A | | | | 2321.0255 | 2324.3 | 2322.0 | |
| I-1259 | A | | | | 2321.0255 | 2324.4 | 2321.8 | |
| I-1260 | A | | | | 2363.0724 | 2366.6 | 2364.3 | |
| I-1261 | A | | | | 2363.0724 | 1183.8 | 1182.1 | |
| I-1262 | A | | | | 2293.0305 | 1148.8 | 1147.0 | |
| I-1263 | A | | | | 2300.9662 | 2304.3 | 2302.7 | |
| I-1264 | A | | | | 2343.0132 | 1173.9 | | |
| I-1265 | A | | | | 2272.9713 | 1138.8 | 1136.2 | |
| I-1266 | A | | | | 2326.9819 | 2330.3 | 2328.5 | |
| I-1267 | A | | | | 2369.0288 | 2372.5 | 2370.3 | |
| I-1268 | A | | | + | 2298.9870 | 1152.0 | 2299.9 | |
| I-1269 | B | | | | 2233.9062 | 1119.0 | 2234.1 | |
| I-1270 | C | | | | 2207.8905 | 1106.0 | 1103.7 | |
| I-1271 | A | | | | 2144.8545 | | | |
| I-1272 | A | | | | 2143.8592 | | | |
| I-1273 | A | | | + | 2158.8701 | 2161.4 | 2159.4 | |
| I-1274 | A | | | + | 2081.8436 | 2084.2 | 2082.0 | |
| I-1275 | A | | | | 2095.8592 | 2098.3 | | |
| I-1276 | A | | | | 2137.8698 | 2140.3 | | |
| I-1277 | A | | | | 2123.8541 | 1063.9 | 1062.0 | |
| I-1278 | A | | | + | 2093.8436 | 2096.3 | 2094.4 | |
| I-1279 | A | | +++ | | 2095.8592 | 2098.4 | 2096.9 | |
| I-1280 | A | | ++ | | 2109.8749 | 2112.4 | 2110.3 | |
| I-1281 | A | | ++ | | 2107.8592 | 2110.4 | | |
| I-1282 | A | | | | 2095.8228 | 2098.3 | 2096.4 | |
| I-1283 | A | | | | 2109.8385 | 1056.8 | 2110.4 | |
| I-1284 | A | | +++ | | 2294.0792 | 2296.6 | 2294.2 | A |
| I-1285 | A | | | | 2338.0690 | 2340.7 | 2339.0 | A |
| I-1286 | B | | | | 2320.0948 | 2322.6 | 2320.3 | A |
| I-1287 | B | | | | 2310.0741 | 2312.8 | 2311.2 | A |
| I-1288 | A | | | | 2354.0639 | 2356.6 | 2354.9 | A |
| I-1289 | B | | | | 2336.0897 | 2338.6 | | A |
| I-1290 | B | D | | + | 2336.1261 | 2338.6 | 2336.4 | A |
| I-1291 | A | D | | + | 2380.1160 | 2282.7 | 2281.1 | A |
| I-1292 | B | | | + | 2362.1418 | 2364.6 | 2362.3 | A |
| I-1293 | B | | | | 2310.0741 | 2312.7 | 2310.7 | A |
| I-1294 | A | | | | 2354.0639 | 2356.5 | | A |
| I-1295 | B | | | | 2336.0897 | 2338.6 | | A |
| I-1296 | B | | | | 2114.9927 | 2118.0 | | A |
| I-1297 | E | | | | 2251.1098 | 2253.6 | 2251.9 | A |
| I-1298 | E | | | | 2265.1254 | 2267.6 | 2265.4 | A |
| I-1299 | B | | +++ | | 2180.0726 | 1092.0 | 1090.4 | A |
| I-1300 | B | | +++ | | 2194.0883 | 2096.5 | 2094.7 | A |
| I-1301 | B | | | | 2090.8980 | 1046.4 | 1044.5 | |
| I-1302 | C | | | | 2090.8980 | 2091.3 | 2089.3 | |
| I-1303 | E | | | | 2106.8752 | 2106.1 | 2104.1 | |
| I-1304 | A | | | | 2063.0114 | 2065.3 | 2063.4 | |
| I-1305 | B | | | | 2082.9801 | 2085.3 | 2083.4 | |
| I-1306 | A | | | | 2036.9594 | 2039.2 | 2037.3 | |
| I-1307 | A | | | | 2074.9614 | 2078.1 | 2076.4 | |
| I-1308 | A | | | | 2094.9301 | 2098.1 | 2096.2 | |
| I-1309 | A | | | | 2048.9094 | 2052.1 | 2050.6 | |
| I-1310 | A | | | | 2048.9958 | 2051.3 | 2049.5 | |
| I-1311 | B | | | | 2068.9645 | 2071.3 | | |
| I-1312 | B | | | | 2068.9645 | 2071.3 | 2069.3 | |
| I-1313 | A | | | | 2022.9437 | 2025.3 | 2023.1 | |
| I-1314 | A | | | | 2060.9458 | 2064.3 | | |
| I-1315 | A | | | | 2080.9145 | 2084.0 | 2081.7 | |
| I-1316 | B | | | | 2034.8937 | 2037.9 | 2036.0 | |
| I-1317 | A | | | | 2060.9958 | 2063.3 | 2061.3 | |
| I-1318 | A | | | | 2075.0114 | 2077.4 | 2075.8 | |
| I-1319 | B | | | | 2089.0271 | 2091.3 | 2089.3 | |
| I-1320 | A | | | | 2072.9458 | 2076.1 | | |
| I-1321 | A | | | | 2086.9614 | 2090.0 | 8087.8 | |
| I-1322 | A | | | | 2100.9771 | 2104.2 | | |
| I-1323 | A | | | | 2046.9801 | 2049.3 | 2047.0 | |
| I-1324 | A | | | | 2060.9958 | 2063.2 | 2061.3 | |
| I-1325 | B | | | | 2075.0114 | 2077.3 | 2075.5 | |
| I-1326 | A | | | | 2058.9301 | 2062.0 | 2060.5 | |
| I-1327 | A | | | | 2072.9458 | 2076.1 | 2074.6 | |
| I-1328 | A | | | | 2086.9614 | 2090.1 | | |
| I-1329 | A | | | | 2075.9737 | 2078.3 | 2076.3 | A |
| I-1330 | C | | | | 2089.9893 | 2092.3 | 2090.0 | A |
| I-1331 | E | | | | 2187.0032 | 2189.4 | 2187.6 | A |
| I-1332 | A | | | | 2101.9893 | 2104.4 | 2101.9 | A |
| I-1333 | C | | | | 2116.0050 | 2118.4 | 2116.2 | A |
| I-1334 | E | | | | 2213.0189 | 2215.3 | 2213.5 | A |
| I-1335 | A | | | | 2072.9458 | 1038.6 | 1036.8 | A |
| I-1336 | B | | | | 2072.9458 | 2076.3 | 2072.8 | A |
| I-1337 | A | | | | 1996.9145 | 1000.6 | 998.8 | A |
| I-1338 | C | | | | 2010.9301 | 1007.6 | 1005.7 | A |
| I-1339 | E | | | | 1995.9304 | 1998.8 | 1995.9 | A |
| I-1340 | C | | | | 2009.9461 | 2012.8 | 2010.8 | A |
| I-1341 | C | | | | 1968.9195 | 1971.7 | 1970.3 | A |
| I-1342 | B | | | | 2114.9927 | 1059.7 | | A |
| I-1343 | B | | | | 2100.9771 | 2104.0 | 2102.0 | A |
| I-1344 | B | | | | 2114.9927 | 1059.8 | | A |
| I-1345 | A | | | + | 2289.1068 | 2291.6 | 2289.9 | |
| I-1346 | A | | | + | 2303.1224 | 2305.6 | 2303.5 | |
| I-1347 | A | | | +++ | 2317.1381 | 2319.6 | 2318.1 | |
| I-1348 | A | | | | 2301.0568 | 2304.4 | 2302.3 | |
| I-1349 | A | | | + | 2315.0724 | 2318.3 | | |
| I-1350 | A | | | + | 2329.0881 | 1166.9 | 1165.2 | |
| I-1351 | A | | | | 2296.9891 | 2300.4 | 2298.5 | |
| I-1352 | A | | | | 2282.9734 | 2286.4 | | |
| I-1353 | A | | | | 2254.9785 | 2258.3 | 2256.6 | |
| I-1354 | A | | | | 2240.9629 | 2244.3 | 2242.8 | |
| I-1355 | A | | | | 2296.0414 | 2299.4 | | |
| I-1356 | A | | | | 2282.0258 | 2285.5 | 2282.8 | |
| I-1357 | A | | | | 2339.0360 | 2342.4 | 2340.0 | |
| I-1358 | A | | | | 2325.0204 | 2328.5 | 2326.8 | |
| I-1359 | A | | | | 2297.0255 | 1150.7 | 1148.8 | |
| I-1360 | A | | | | 2283.0098 | 2286.3 | | |
| I-1361 | A | | | | 2338.0884 | 2341.5 | | |
| I-1362 | A | | | | 2324.0727 | 2327.5 | | |
| I-1363 | B | | +++ | | 2102.9886 | 2105.4 | 2103.5 | |
| I-1364 | B | | +++ | | 2074.9573 | 2077.1 | | |
| I-1365 | A | | +++ | | 2157.8749 | 2060.2 | 2158.1 | |
| I-1366 | C | | +++ | | 2171.8905 | 1087.9 | 1086.2 | |
| I-1367 | A | | | + | 1995.8617 | 1999.0 | 1997.4 | |
| I-1368 | A | | | | 1995.8617 | 1999.1 | | |
| I-1369 | A | | | + | 2009.8773 | 2013.0 | | |
| I-1370 | A | | | + | 2009.8773 | 2012.9 | 2011.5 | |
| I-1371 | A | | | +++ | 2009.8773 | 2013.0 | 2011.4 | |
| I-1372 | A | | | + | 2009.8773 | 2013.0 | 2011.1 | |
| I-1373 | A | | | +++ | 2023.8930 | 2027.0 | | |
| I-1374 | A | | | | 2023.8930 | 1014.2 | 2025.2 | |
| I-1375 | A | | | | 2023.8930 | 2027.1 | 2025.5 | |
| I-1376 | A | | | + | 2024.8519 | 2028.0 | 2026.2 | |
| I-1377 | A | | | | 2024.8519 | 2027.9 | 2026.0 | |
| I-1378 | A | | | | 2038.8675 | 2042.0 | 2040.5 | |
| I-1379 | A | | | | 2050.8675 | 2054.0 | 2052.7 | |
| I-1380 | A | | | | 2064.8832 | 2068.7 | 2066.8 | |
| I-1381 | A | | | | 2064.8832 | 2068.0 | 2066.6 | |
| I-1382 | A | | | | 2064.8832 | 2068.1 | 2066.2 | |
| I-1383 | A | | | | 2064.8832 | 2068.0 | | |
| I-1384 | A | | | | 2064.8832 | 2068.0 | 2066.3 | |
| I-1385 | A | | | | 2064.8832 | 2068.0 | 2066.4 | |
| I-1386 | B | | | | 2157.8749 | 2160.4 | 2158.6 | |
| I-1387 | B | | | | 2158.8701 | 2161.4 | 2159.7 | |
| I-1388 | B | | | | 2095.8592 | 2098.2 | 2096.5 | |
| I-1389 | B | | | | 2107.8592 | 2110.4 | | |
| I-1390 | B | | | | 2171.8905 | 2174.3 | 2172.6 | |
| I-1391 | B | | | | 2137.8698 | 2140.4 | 2138.2 | |
| I-1392 | A | | | | 2316.0635 | 2318.6 | | |
| I-1393 | A | | | | 2308.0414 | 2311.3 | 2309.8 | |
| I-1394 | A | | | | 2387.1006 | 2389.7 | 2388.2 | |
| I-1395 | A | | | | 2379.0786 | 1192.0 | 1190.3 | |
| I-1396 | A | | | | 2011.9212 | 2014.2 | 2011.9 | |
| I-1397 | C | | | | 2067.9474 | 2070.5 | 2068.2 | |
| I-1398 | A | | | | 2207.9160 | 2210.4 | 2208.8 | |
| I-1399 | A | | | | 2137.9505 | 2140.4 | | |
| I-1400 | A | | | | 2088.9365 | 2091.4 | 2089.3 | |
| I-1401 | A | | | | 2088.9365 | 2091.3 | 2089.3 | |
| I-1402 | B | | | | 2492.1684 | 1248.1 | 1246.2 | |
| I-1403 | A | | | | 2668.2733 | 1336.1 | 1334.8 | |
| I-1404 | A | | | | 3020.4830 | 1512.4 | 1510.7 | |
| I-1405 | B | | | | 2484.1463 | 2487.6 | 2485.6 | |
| I-1406 | A | | | | 2660.2512 | 1332.5 | 1330.8 | |
| I-1407 | A | | | | 3012.4609 | 1508.8 | 1506.9 | |
| I-1408 | A | | | | 2739.3104 | 1371.7 | 1370.2 | |
| I-1409 | A | | | | 3091.5201 | 1547.9 | 1545.8 | |
| I-1410 | A | | | | 4016.0706 | 2010.5 | 2008.5 | |
| I-1411 | A | | | | 2555.1834 | 1280.0 | 1278.1 | |
| I-1412 | A | | | | 2731.2883 | 1368.1 | 1366.7 | |
| I-1413 | A | | | | 3083.4980 | 1544.3 | 1542.8 | |
| I-1414 | A | | | | 2066.8988 | 2070.0 | 2068.5 | |
| I-1415 | A | | | | 2052.8832 | 2056.0 | 2053.3 | |
| I-1416 | A | | | | 2038.8675 | 2041.9 | 2039.4 | |
| I-1417 | A | | | | 2038.8675 | 2041.9 | | |
| I-1418 | A | | | | 2038.8675 | 2042.0 | 2040.3 | |
| I-1419 | A | | | | 2050.8675 | 2054.0 | 2052.4 | |
| I-1420 | A | | | | 2036.8519 | 2039.9 | 2038.1 | |
| I-1421 | A | | | | 2050.8675 | 2054.0 | 2052.4 | |
| I-1422 | A | | | | 2036.8519 | 2054.0 | 2052.4 | |
| I-1423 | A | | | | 2050.8675 | 2054.0 | | |
| I-1424 | A | | | | 2036.8519 | 2040.0 | 2038.5 | |
| I-1425 | B | | | | 2172.8858 | 2175.6 | 2174.8 | |
| I-1426 | B | | | | 2185.9062 | 1094.8 | 1093.7 | |
| I-1427 | B | | | | 2109.8749 | 2112.4 | 2110.4 | |
| I-1428 | C | | | | 2123.8905 | 1063.8 | 2124.4 | |
| I-1429 | A | | | | 2121.8749 | 2123.3 | | |
| I-1430 | B | | | | 2123.8541 | 2126.7 | 2124.5 | |
| I-1431 | A | | | | 2069.9267 | 2072.3 | 2070.4 | |
| I-1432 | A | | | | 2055.9111 | 2058.3 | 2056.1 | |
| I-1433 | A | | | | 2083.9424 | 2086.3 | 2083.9 | |
| I-1434 | A | | | | 2111.9737 | 2114.3 | 2112.7 | |
| I-1435 | A | | | | 2083.9424 | 2086.3 | 2084.7 | |
| I-1436 | A | | | | 2111.9737 | 2114.4 | 2112.5 | |
| I-1437 | B | | | | 2102.9059 | 2105.3 | 2103.2 | |
| I-1438 | A | | | | 2132.9165 | 2135.4 | | |
| I-1439 | A | | | | 2132.9165 | 2135.4 | 2133.2 | |
| I-1440 | A | | | | 2139.9587 | 2142.4 | 2140.6 | |
| I-1441 | A | | | | 2139.9587 | 2142.4 | 2140.0 | |
| I-1442 | A | | | | 2139.9587 | 2142.3 | 2140.8 | |
| I-1443 | B | | | | 2111.9274 | 1058.3 | 2112.4 | |
| I-1444 | B | | | | 2151.9587 | 1263.3 | | |
| I-1445 | B | | | | 2151.9587 | 2154.2 | | |
| I-1446 | B | | | | 2151.9587 | 2154.4 | 2152.3 | |
| I-1447 | B | | | | 2151.9587 | 1078.8 | | |
| I-1448 | B | | | | 2137.9430 | 2140.2 | | |
| I-1449 | B | | | | 2144.9529 | 2147.4 | 2145.3 | |
| I-1450 | B | | | | 2116.9216 | 2119.4 | 2117.3 | |
| I-1451 | A | | | | 2144.8545 | 2147.3 | 2145.1 | |
| I-1452 | A | C | ++ | | 2126.9927 | 2125.9 | | A |
| I-1453 | A | | | | 2173.8698 | 2176.2 | 2174.4 | |
| I-1454 | A | | ++ | | 2172.8858 | 2175.2 | 2173.2 | |
| I-1455 | A | | | | 2172.8858 | 2176.1 | 2174.1 | |
| I-1456 | B | | +++ | | 2201.9011 | 2005.1 | 2002.6 | |
| I-1457 | B | | ++ | | 2123.8905 | 1064.3 | 2124.8 | |
| I-1458 | A | | ++ | | 2121.8749 | 2124.4 | 2122.9 | |
| I-1459 | B | | | | 2109.8749 | 2111.0 | 2111.0 | |
| I-1460 | A | | | | 2137.8698 | 2139.1 | 2139.1 | |
| I-1461 | A | | | | 2158.8701 | 2160.1 | 2160.1 | |
| I-1462 | B | | | | 2107.8592 | 2109.0 | 2109.0 | |
| I-1463 | A | D | ++ | | 2140.9356 | 2143.8 | 2141.5 | |
| I-1464 | A | | ++ | | 2124.9771 | 1064.6 | | |
| I-1465 | A | D | ++ | | 2082.9301 | 2085.8 | 2084.2 | |
| I-1466 | A | D | ++ | | 2110.9614 | 1057.6 | | |
| I-1467 | A | | ++ | | 2154.9512 | 2156.5 | | |
| I-1468 | A | | +++ | | 2096.9458 | 2099.8 | | |
| I-1469 | A | | | | 2186.9014 | | | |
| I-1470 | B | | | | 2186.9014 | | | |
| I-1471 | B | | | | 2123.8905 | | | |
| I-1472 | A | | | | 2263.0006 | 2263.8 | 2262.1 | |
| I-1473 | A | | | | 2305.0476 | 2305.9 | 2304.4 | |
| I-1474 | A | | | | 2402.1003 | 1201.6 | 1099.7 | |
| I-1475 | A | | | | 2293.0112 | 2293.9 | 2292.4 | |
| I-1476 | A | | | | 2390.0639 | 2391.2 | 2389.7 | |
| I-1477 | A | | | | 2289.0163 | 2289.9 | 2288.4 | |
| I-1478 | A | | | | 2261.0213 | 2261.9 | 2260.2 | |
| I-1479 | A | | | | 2303.0683 | 2303.9 | 2302.5 | |
| I-1480 | A | | | | 2400.1210 | 2401.1 | 2399.6 | |
| I-1481 | A | | | | 2291.0319 | 2291.8 | 2290.4 | |
| I-1482 | A | | | | 2388.0847 | 2389.2 | 2387.7 | |
| I-1483 | A | | | | 2287.0370 | 2287.9 | 2285.7 | |
| I-1484 | E | | | | 2032.8739 | 2033.6 | 2031.4 | |
| I-1485 | E | D | | | 2036.9052 | 2037.4 | 2035.7 | A |
| I-1486 | B | | | | 2060.9052 | 2062.7 | 2060.3 | |
| I-1487 | B | | | | 2060.9052 | 1031.9 | 1030.0 | |
| I-1488 | A | | | + | 2074.9209 | 2077.0 | 2074.9 | |
| I-1489 | A | | | | 2074.9209 | 2076.9 | 2074.9 | |
| I-1490 | B | | | | 2088.9365 | 2091.1 | 2088.6 | |
| I-1491 | A | | | | 2088.9365 | 2091.0 | 2088.7 | |
| I-1492 | B | | | | 2004.8426 | 2006.7 | 2004.4 | |
| I-1493 | E | | | | 2018.8583 | 1011.0 | 1009.0 | |
| I-1494 | A | | | | 2032.8739 | 2034.9 | 2032.9 | |
| I-1495 | A | | | | 2032.8739 | 1017.8 | 1015.9 | |
| I-1496 | A | | +++ | +++ | 2064.9365 | 2067.2 | 2065.6 | A |
| I-1497 | A | | +++ | +++ | 2078.9522 | 2081.1 | 2079.6 | A |
| I-1498 | C | | ++ | + | 2092.9678 | 2095.1 | 2092.5 | A |
| I-1499 | A | | +++ | | 2008.8739 | 2010.8 | 2008.8 | A |
| I-1500 | A | | +++ | | 2022.8896 | 2024.9 | 2023.0 | A |
| I-1501 | A | | | | 2036.9052 | 1019.9 | 1017.9 | A |
| I-1502 | A | | | | 2069.8540 | 2072.9 | 2070.7 | |
| I-1503 | A | | | | 2055.8384 | 2058.9 | 2057.2 | |
| I-1504 | A | | | | 2095.8697 | 1049.9 | 1048.0 | |
| I-1505 | A | | | + | 2055.8384 | 1030.4 | 1028.0 | |
| I-1506 | A | | | + | 2041.8227 | 1022.8 | 1020.7 | |
| I-1507 | A | | | | 2081.8540 | 2085.0 | 2083.3 | |
| I-1508 | A | | | + | 2009.8773 | 1006.6 | 1004.6 | |
| I-1509 | A | | | | 2009.8773 | 2012.0 | 2010.3 | |
| I-1510 | C | | | | 1995.8617 | 988.2 | 986.1 | |
| I-1511 | C | | | | 2035.8930 | 1008.6 | 1006.8 | |
| I-1512 | C | | | | 2009.8773 | 995.3 | 993.3 | |
| I-1513 | C | | | | 1995.8617 | 988.2 | 986.1 | |
| I-1514 | C | | | | 2035.8930 | 2038.6 | | |
| I-1515 | C | | | | 2049.9086 | 1015.0 | 1013.3 | |
| I-1516 | C | | | | 2035.8930 | 1008.3 | 1006.4 | |
| I-1517 | C | | | | 2035.8930 | 1008.3 | 1006.4 | |
| I-1518 | A | | | | 2021.8773 | 1012.7 | 2022.9 | |
| I-1519 | A | | | | 2159.8541 | 2162.0 | 2160.6 | |
| I-1520 | A | | | | 2160.8494 | 2163.2 | 2160.9 | |
| I-1521 | A | | | | 2111.8541 | 2113.2 | 2111.1 | |
| I-1522 | A | | | | 2097.8385 | 2099.7 | 2097.3 | |
| I-1523 | A | | | | 2114.9927 | 1061.4 | 2119.7 | A |
| I-1524 | B | | | | 2141.0084 | 1075.5 | 1073.5 | A |
| I-1525 | A | | | | 5122.6777 | 1025.7 | | |
| I-1526 | A | | | | 7764.2506 | 1554.1 | | |
| I-1527 | A | | | + | 2057.9040 | 2060.3 | 2057.8 | |
| I-1528 | A | | | + | 2043.8884 | 2046.3 | 2044.2 | |
| I-1529 | A | | | | 2083.9197 | 1043.6 | 1041.4 | |
| I-1530 | A | | | + | 2043.8884 | 2046.7 | 2044.2 | |
| I-1531 | A | | | + | 2029.8727 | 2032.6 | 2030.3 | |
| I-1532 | B | | | | 2085.9086 | 2088.8 | 2085.5 | |
| I-1533 | C | | | | 2085.9086 | 2088.9 | 2086.8 | |
| I-1534 | A | | | | 2071.8930 | 2074.8 | 2072.9 | |
| I-1535 | B | | | | 2071.8930 | 2074.9 | 2073.2 | |
| I-1536 | B | | | | 2073.9587 | 2075.6 | 2073.3 | |
| I-1537 | A | | | + | 2027.8679 | 2030.3 | 2029.1 | |
| I-1538 | A | | | | 2027.8679 | 2030.3 | 2029.5 | |
| I-1539 | A | | | + | 2013.8523 | 2016.1 | 2013.9 | |
| I-1540 | A | | | | 2013.8523 | 2016.2 | 2013.7 | |
| I-1541 | A | | | | 2015.9179 | 2017.3 | 2015.3 | |
| I-1542 | A | | | | 2015.9179 | 2017.4 | 2015.3 | |
| I-1543 | A | | | | 2001.9023 | 2003.4 | 2001.1 | |
| I-1544 | A | | | | 2001.9023 | 2003.4 | 2001.2 | |
| I-1545 | C | | | | 2011.9430 | 1006.8 | 1004.4 | |
| I-1546 | A | | | | 1997.9274 | 1999.4 | 1997.2 | |
| I-1547 | A | | | | 1997.9274 | 1999.5 | 1996.8 | |
| I-1548 | A | | | | 2019.8651 | 2022.2 | 2020.9 | A |
| I-1549 | B | | | +++ | 1963.8025 | 1967.2 | 1965.2 | A |
| I-1550 | A | | | +++ | 2005.8494 | 2008.6 | 2006.3 | A |
| I-1551 | A | | | +++ | 1991.8338 | 1994.3 | 1992.1 | A |
| I-1552 | A | | | ++ | 2005.8494 | 2008.7 | 2006.5 | A |
| I-1553 | A | | | +++ | 2039.8338 | 2042.6 | 2039.5 | A |
| I-1554 | A | | | | 2031.8651 | 2034.2 | 2032.6 | A |
| I-1555 | A | | | | 2045.8807 | 2048.0 | 2046.0 | A |
| I-1556 | A | | | | 2115.8651 | 2118.1 | 2116.6 | A |
| I-1557 | C | | | | 2018.8810 | 2021.2 | 2019.0 | A |
| I-1558 | D | | | | 1991.8701 | 1994.1 | 1991.5 | A |
| I-1559 | A | | | | 2001.8181 | 2007.2 | 2005.2 | A |
| I-1560 | A | | | + | 2023.8930 | 2026.4 | 2024.5 | |
| I-1561 | A | | | + | 2023.8930 | 1013.8 | 2024.4 | |
| I-1562 | A | | | + | 2037.9086 | 2040.4 | 2038.5 | |
| I-1563 | A | | | | 2037.9086 | 2041.7 | 2039.4 | |
| I-1564 | A | | | + | 2035.8930 | 2038.3 | 2036.9 | |
| I-1565 | A | | | + | 2035.8930 | 1019.8 | 1017.4 | |
| I-1566 | A | | | | 2049.9086 | 1026.8 | | |
| I-1567 | A | | | | 2049.9086 | 2053.1 | 2049.8 | |
| I-1568 | A | | | + | 2063.9243 | 2066.6 | 2064.7 | |
| I-1569 | A | | | + | 2063.9243 | 2066.6 | 2064.4 | |
| I-1570 | A | | | | 2079.9192 | 1041.8 | 1039.7 | |
| I-1571 | A | | | | 2079.9192 | 1041.8 | | |
| I-1572 | A | | | + | 2055.8651 | 2058.6 | 2056.0 | |
| I-1573 | A | | | + | 2055.8651 | 2058.5 | 2056.4 | |
| I-1574 | A | | | + | 2069.8807 | 2072.6 | 2070.5 | |
| I-1575 | A | | | + | 2069.8807 | 2072.5 | 2070.0 | |
| I-1576 | A | | | | 2069.8807 | 1065.7 | 1063.7 | |
| I-1577 | A | | | + | 2067.8651 | 2070.6 | 2068.7 | |
| I-1578 | A | | | + | 2067.8651 | 2070.5 | 2068.6 | |
| I-1579 | A | | | + | 2081.8807 | 1042.8 | 1040.6 | |
| I-1580 | A | | | + | 2095.8964 | 2098.6 | 2096.0 | |
| I-1581 | A | | | + | 2111.8913 | 2114.9 | 2112.8 | |
| I-1582 | A | | | | 2015.8338 | 1009.8 | 1007.9 | |
| I-1583 | A | | | | 2015.8338 | 1009.8 | 1007.8 | |
| I-1584 | A | | | | 2029.8494 | 2032.2 | 2030.4 | |
| I-1585 | A | | | | 2029.8494 | 2032.7 | 2030.3 | |
| I-1586 | A | | | | 2027.8338 | 2030.4 | 2027.9 | |
| I-1587 | A | | | | 2027.8338 | 2030.5 | 2028.1 | |
| I-1588 | A | | | | 2041.8494 | 2044.6 | 2042.3 | |
| I-1589 | A | | | | 2041.8494 | 1022.8 | 1020.7 | |
| I-1590 | A | | | | 2055.8651 | 2058.6 | 2056.4 | |
| I-1591 | A | | | | 2055.8651 | 1029.8 | 1027.9 | |
| I-1592 | A | | | | 2071.8600 | 1037.8 | 1035.3 | |
| I-1593 | A | | | | 2071.8600 | 2074.5 | 2072.1 | |
| I-1594 | A | | | | 1927.9396 | 1929.5 | 1927.3 | |
| I-1595 | A | | | | 1927.9396 | 1929.5 | 1927.4 | |
| I-1596 | B | | | | 1961.9007 | 1963.8 | 1961.6 | |
| I-1597 | B | | | | 1961.9007 | 1964.0 | 1961.8 | |
| I-1598 | A | | | + | 1961.9007 | 1963.8 | 1962.0 | |
| I-1599 | A | | | | 1961.9007 | 1963.9 | 1962.0 | |
| I-1600 | A | | | | 1961.9007 | 982.4 | 980.6 | |
| I-1601 | A | | | | 1941.9553 | 1943.4 | 1941.3 | |
| I-1602 | B | | | | 2005.8502 | 2008.4 | 2005.8 | |
| I-1603 | C | | | | 2005.8502 | 1004.7 | 2006.7 | |
| I-1604 | A | | | + | 2005.8502 | 2008.4 | 2006.0 | |
| I-1605 | A | | | | 2005.8502 | 1004.6 | 1002.8 | |
| I-1606 | A | | | | 2005.8502 | 1004.6 | 1002.6 | |
| I-1607 | A | | | | 2005.8502 | 1004.7 | 1002.8 | |
| I-1608 | A | | | | 1995.9270 | 1997.4 | 1995.3 | |
| I-1609 | A | | | | 1995.9270 | 1997.5 | 1995.2 | |
| I-1610 | A | | | | 1995.9270 | 1997.5 | 1995.2 | |
| I-1611 | A | | | | 1995.9270 | 1997.4 | 1995.3 | |
| I-1612 | A | | | | 2011.8066 | 1007.7 | 1005.7 | |
| I-1613 | A | | | | 2011.8066 | 1007.7 | 1005.9 | |
| I-1614 | A | | | | 2001.8834 | 2003.3 | 2001.3 | |
| I-1615 | A | | | | 2001.8834 | 2003.4 | 2001.1 | |
| I-1616 | A | | | | 2112.9771 | 2115.4 | 2113.6 | A |
| I-1617 | A | | | | 2138.9927 | 1071.2 | 1069.3 | A |
| I-1618 | B | | | | 2141.0084 | 1072.2 | 1070.1 | A |
| I-1619 | A | | | | 2170.9825 | 1087.2 | 1085.7 | A |
| I-1620 | A | | | | 2098.9614 | 2101.0 | 2099.3 | A |
| I-1621 | A | | | | 2124.9771 | 1064.1 | 1061.9 | A |
| I-1622 | A | | | | 2126.9927 | 2129.2 | 2127.4 | A |
| I-1623 | A | | | | 2156.9669 | 2160.0 | 2157.9 | A |
| I-1624 | A | | | | 2126.9927 | 1065.0 | 1063.0 | A |
| I-1625 | A | | | | 2112.9771 | 2115.5 | 2113.3 | A |
| I-1626 | A | | | | 2098.9614 | 1051.2 | 1049.3 | A |
| I-1627 | A | | | | 2084.9458 | 2087.4 | 2085.5 | A |
| I-1628 | A | | | | 2194.0097 | 2195.6 | 2193.5 | |
| I-1629 | A | | | | 2194.0097 | 2195.6 | 2193.4 | |
| I-1630 | A | | | | 2136.8654 | 2139.4 | 2137.3 | |
| I-1631 | B | | | | 2123.8701 | 1063.8 | 1062.0 | |
| I-1632 | A | | | | 2136.8654 | 1070.2 | | |
| I-1633 | B | | | | 2123.8701 | 1063.8 | 1061.7 | |
| I-1634 | A | | | | 2139.8651 | 1071.7 | 1069.7 | |
| I-1635 | E | | | | 2127.8287 | 1814.1 | 1811.7 | |
| I-1636 | A | | | | 2138.8810 | 2141.5 | 2138.8 | |
| I-1637 | B | | | | 2125.8858 | 2128.7 | 2126.2 | |
| I-1638 | A | | | + | 2138.8810 | 2041.9 | 2039.3 | |
| I-1639 | B | | | + | 2125.8858 | 1064.8 | | |
| I-1640 | E | | | | 2199.8804 | 1085.8 | 1084.0 | |
| I-1641 | A | | | | 2170.9881 | 2173.0 | 2171.0 | |
| I-1642 | A | | | | 2170.9881 | 2172.9 | 2171.4 | |
| I-1643 | A | | | | 2128.9412 | 2130.9 | 2128.7 | |
| I-1644 | A | | | | 2128.9412 | 1065.9 | 1064.0 | |
| I-1645 | A | | | | 2137.0271 | 2138.5 | 2136.5 | |
| I-1646 | A | | | | 2137.0271 | 2138.5 | 2136.4 | |
| I-1647 | A | | | | 2094.9801 | 2096.4 | 2094.4 | |
| I-1648 | A | | | | 2023.9430 | 2025.3 | 2023.4 | |
| I-1649 | A | | | | 2023.9430 | 2025.4 | 2023.3 | |
| I-1650 | A | | | | 2155.0177 | 2156.6 | 2154.4 | |
| I-1651 | A | | | | 2155.0177 | 2156.5 | 2154.1 | |
| I-1652 | A | | | | 2112.9707 | 2114.4 | 2112.6 | |
| I-1653 | A | | | | 2041.9336 | 2043.3 | 2041.4 | |
| I-1654 | B | | | + | 2205.0145 | 2206.4 | 2204.3 | |
| I-1655 | B | | | + | 2205.0145 | 2206.5 | 2204.2 | |
| I-1656 | A | | | + | 2162.9675 | 2164.6 | 2162.3 | |
| I-1657 | A | | | + | 2162.9675 | 2164.5 | 2162.1 | |
| I-1658 | A | | | + | 2091.9304 | 2093.3 | 2091.3 | |
| I-1659 | A | | | + | 2091.9304 | 2093.4 | 2091.2 | |
| I-1660 | A | | | | 2202.0172 | 2203.4 | 2201.3 | |
| I-1661 | C | | | | 2202.0172 | 1102.3 | 1100.3 | |
| I-1662 | B | | | | 2188.0016 | 2189.4 | 2187.3 | |
| I-1663 | D | | | | 2188.0016 | 1095.3 | 1093.4 | |
| I-1664 | A | | | | 2264.0329 | 2265.5 | 2263.3 | |
| I-1665 | B | | | | 2264.0329 | 2265.5 | 2263.7 | |
| I-1666 | A | | | | 2224.0703 | 2225.6 | 2223.5 | |
| I-1667 | B | | | | 2242.0234 | 2243.5 | 2241.4 | |
| I-1668 | A | | | | 2242.0234 | 1122.2 | 1120.4 | |
| I-1669 | A | | | | 2242.0234 | 2243.5 | 2241.6 | |
| I-1670 | A | | | | 2203.0125 | 2204.5 | 2202.4 | |
| I-1671 | C | | | | 2203.0125 | 2204.5 | 2202.4 | |
| I-1672 | A | | | | 2229.0281 | 2230.5 | 2228.5 | |
| I-1673 | A | | | | 2130.9512 | 1067.1 | 1065.0 | A |
| I-1674 | C | | | | 2129.9672 | 2132.4 | 2130.0 | A |
| I-1675 | C | | | | 2102.9563 | 1053.0 | 1051.2 | A |
| I-1676 | C | | | | 2144.9669 | 1074.2 | 1072.3 | A |
| I-1677 | C | | | | 2143.9829 | 1073.6 | 1071.7 | A |
| I-1678 | C | | | | 2116.9720 | 1060.1 | 1058.1 | A |
| I-1679 | B | | | | 2158.9825 | 1081.1 | 1079.3 | A |
| I-1680 | A | | | | 2126.9 | 2128.2 | 2125.8 | |
| I-1681 | A | | | | 2099.9 | 2101.3 | 2098.9 | |
| I-1682 | C | | | | 2113.9 | 2115.1 | 2113.0 | |
| I-1683 | C | | | | 2099.9 | 2101.2 | 2099.0 | |
| I-1684 | C | | | | 2113.9 | 2115.2 | 2113.3 | |
| I-1685 | C | | | | 2113.9 | 2115.1 | 2113.8 | |
| I-1686 | B | | | | 2113.9 | 2115.2 | 2113.1 | |
| I-1687 | C | | | | 2119.9 | 2121.2 | 2119.3 | |
| I-1688 | A | | | | 2211.0 | 1107.1 | 1105.3 | A |
| I-1689 | A | | | + | 2197.0 | 2199.0 | 2197.3 | A |
| I-1690 | A | | | + | 2225.0 | 2228.3 | 2226.0 | A |
| I-1691 | A | | | | 2211.0 | 2212.9 | 2210.3 | A |
| I-1692 | A | | | + | 2183.0 | 2185.2 | 2183.6 | A |
| I-1693 | A | | | + | 2168.9 | 2170.6 | 2169.0 | A |
| I-1694 | A | | | | 2195.0 | 1099.0 | 1097.2 | A |
| I-1695 | A | | | + | 2223.0 | 2224.9 | 2222.8 | A |
| I-1696 | A | | | | 2209.0 | 2210.7 | 2208.7 | |
| I-1697 | A | | | | 1985.9 | 1987.7 | | A |
| I-1698 | A | | | | 1971.9 | 1973.8 | 1971.6 | A |
| I-1699 | A | | | | 1985.9 | 1987.8 | 1985.5 | A |
| I-1700 | A | | | | 1971.9 | 1973.7 | 1971.5 | A |
| I-1701 | A | | | + | 2620.1 | 1311.9 | 1309.9 | |
| I-1702 | A | | | + | 3896.9 | 1950.3 | 1948.7 | |
| I-1703 | A | C | | + | 2762.2 | 1382.8 | 1381.0 | |
| I-1704 | A | D | | + | 4039.0 | 2021.3 | 2019.2 | |
| I-1705 | A | C | | + | 2833.2 | 1418.4 | 1416.2 | |
| I-1706 | A | D | | + | 4110.0 | 2056.9 | 2054.8 | |
| I-1707 | A | | | + | 2751.3 | 1377.5 | 1375.3 | |
| I-1708 | A | D | | + | 4028.0 | 2016.0 | 2014.2 | |
| I-1709 | A | | ++ | | 2004.9 | 2006.2 | 2004.0 | |
| I-1710 | A | | | + | 2165.9 | 1084.4 | 1082.3 | |
| I-1711 | A | | | | 2179.9 | 2181.6 | 2179.9 | |
| I-1712 | A | C | | + | 2163.9 | 2165.4 | 2162.6 | |
| I-1713 | A | C | | + | 2177.9 | 2180.7 | 2178.1 | |
| I-1714 | A | C | | + | 2191.9 | 2193.6 | 2191.6 | |
| I-1715 | A | | | | 2213.9 | 2215.6 | 2212.8 | |
| I-1716 | A | | | | 2191.8 | 2193.5 | | |
| I-1717 | A | B | | + | 2264.9 | 2266.5 | 2264.6 | |
| I-1718 | A | | | | 2204.9 | 2205.6 | 2203.5 | |
| I-1719 | A | | | | 2215.9 | 2216.9 | 2214.6 | |
| I-1720 | A | | | | 2240.9 | 2141.8 | 2140.2 | |
| I-1721 | A | | | | 2242.9 | 2244.6 | 2243.1 | |
| I-1722 | A | | | | 2201.9 | 2204.3 | | |
| I-1723 | A | C | | + | 2164.9 | 2266.5 | 2263.7 | |
| I-1724 | A | | | +++ | 2178.9 | 2180.6 | 2179.2 | |
| I-1725 | A | C | | +++ | 2162.9 | 2164.6 | | |
| I-1726 | A | | | +++ | 2176.9 | 2179.5 | 2177.7 | |
| I-1727 | A | | | +++ | 2190.9 | 2193.8 | 2191.1 | |
| I-1728 | B | | | | 2212.9 | 2215.6 | 2213.5 | |
| I-1729 | A | | | | 2190.8 | 2193.4 | 2191.6 | |
| I-1730 | A | | | +++ | 2263.9 | 2265.5 | 2264.5 | |
| I-1731 | A | | | | 2203.9 | 2205.7 | | |
| I-1732 | A | | | +++ | 2239.9 | 2241.5 | 2239.2 | |
| I-1733 | A | | | +++ | 2241.9 | 2243.7 | 2241.7 | |
| I-1734 | A | | | | 2200.9 | 2203.6 | 2201.6 | |
| I-1735 | C | | | | 2084.0 | 2086.1 | 2084.4 | |
| I-1736 | A | | | + | 2084.0 | 2086.2 | 2084.7 | |
| I-1737 | A | | | | 2193.0 | 2195.1 | 2193.8 | |
| I-1738 | A | | | | 2207.0 | 2209.1 | 2207.8 | |
| I-1739 | A | | | | 2253.0 | 2255.1 | 2253.6 | |
| I-1740 | A | | | + | 2203.0 | 2205.4 | 2203.5 | |
| I-1741 | A | | | | 2217.0 | 2217.8 | 2215.8 | |
| I-1742 | A | | | ++ | 2217.0 | 2219.1 | 2217.7 | |
| I-1743 | A | | | ++ | 2217.0 | 2219.2 | 2217.8 | |
| I-1744 | C | | | | 2170.0 | 2172.1 | 2170.4 | |
| I-1745 | B | | | | 2232.0 | 1117.7 | 1116.2 | |
| I-1746 | D | | | | 2198.0 | 1100.6 | 1198.9 | |
| I-1747 | A | | | | 2169.0 | 2171.2 | 2169.6 | |
| I-1748 | C | | | | 2169.0 | 2171.1 | 2169.1 | |
| I-1749 | A | | | | 2183.0 | 2182.8 | 2180.9 | |
| I-1750 | B | | | | 2197.1 | 1100.0 | 1098.5 | |
| I-1751 | A | | | | 2242.0 | 1122.9 | 1120.9 | |
| I-1752 | A | C | | + | 2229.0 | 1116.3 | 1114.4 | |
| I-1753 | A | | | +++ | 2211.0 | 2213.1 | 2212.0 | |
| I-1754 | A | | | +++ | 2189.0 | 2191.2 | | |
| I-1755 | A | | | +++ | 2190.0 | 2192.2 | 2190.6 | |
| I-1756 | A | | | | 2190.0 | 1096.4 | 1094.6 | |
| I-1757 | A | | | | 1995.9 | | | |
| I-1758 | A | | | | 2009.9 | | | |
| I-1759 | B | | | | 2066.9 | | | |
| I-1760 | A | | | | 2009.9 | | | |
| I-1761 | A | | | | 2023.9 | | | |
| I-1762 | A | | | | 2080.9 | | | |
| I-1763 | B | | | | 2207.0 | 1104.9 | 1103.4 | |
| I-1764 | A | | | | 2207.0 | 2209.2 | 2207.7 | |
| I-1765 | A | | | | 2207.0 | 1105.1 | 1103.9 | |
| I-1766 | A | D | | +++ | 2187.9 | | | |
| I-1767 | A | | | | 2296.9 | | | |
| I-1768 | A | | | | 2285.0 | | | |
| I-1769 | B | D | | + | 2313.0 | | | |
| I-1770 | B | D | | + | 2301.0 | | | |
| I-1771 | A | | | | 2286.9 | | | |
| I-1772 | A | D | | + | 2275.0 | | | |
| I-1773 | A | D | | + | 2298.9 | | | |
| I-1774 | A | C | | + | 2287.0 | | | |
| I-1775 | A | | | | 2287.0 | | | |
| I-1776 | A | | | | 2346.9 | | | |
| I-1777 | B | D | | + | 2335.0 | | | |
| I-1778 | A | | | | 2066.9 | | | |
| I-1779 | A | | | | 1981.8 | | | |
| I-1780 | A | | | | 1981.8 | 992.3 | 990.3 | |
| I-1781 | A | | | | 1995.9 | | | |
| I-1782 | A | | | | 2052.9 | | | |
| I-1783 | A | | | | 2211.0 | 2212.7 | 2210.0 | |
| I-1784 | A | C | | | 2227.0 | 2229.1 | 2227.0 | |
| I-1785 | A | | | | 2211.0 | 2212.7 | 2210.6 | |
| I-1786 | A | C | | | 2031.9 | 2033.8 | 2031.7 | |
| I-1787 | A | | ++ | + | 2569.1 | 1286.3 | 1284.3 | |
| I-1788 | A | | ++ | + | 2833.2 | 1418.3 | 1416.3 | |
| I-1789 | A | | ++ | + | 2653.2 | 1328.3 | 1326.3 | |
| I-1790 | A | | ++ | + | 2917.3 | 1460.4 | 1458.3 | |
| I-1791 | E | | + | + | 2793.3 | 1398.4 | | |
| I-1792 | E | | + | + | 3057.5 | 1530.5 | | |
| I-1793 | B | | | | 2080.9 | 2082.9 | 2080.6 | |
| I-1794 | C | | | | 2038.9 | 2040.8 | 2039.0 | |
| I-1795 | C | | | | 2066.9 | 2068.3 | 2065.7 | |
| I-1796 | A | | | | 2094.9 | 2097.0 | 2095.5 | |
| I-1797 | A | D | | | 2052.9 | 2054.8 | | |
| I-1798 | A | C | | | 2080.9 | 1042.0 | | |
| I-1799 | B | | | | 2094.9 | 2096.2 | 2094.5 | |
| I-1800 | C | | | | 2052.9 | 2055.9 | 2053.6 | |
| I-1801 | E | | | | 2080.9 | 2083.7 | 2082.1 | |
| I-1802 | B | | | | 2094.9 | 2096.8 | | |
| I-1803 | C | | | | 2052.9 | 2055.0 | 2053.4 | |
| I-1804 | C | | | | 2080.9 | 2082.9 | 2080.9 | |
| I-1805 | A | C | | | 2324.0 | 1163.5 | 1161.3 | |
| I-1806 | A | C | | | 2289.0 | 1146.0 | 1144.1 | |
| I-1807 | A | D | | | 2199.0 | 2200.8 | 2198.7 | |
| I-1808 | A | D | | | 2245.0 | 1124.0 | 1121.6 | |
| I-1809 | A | B | | | 2097.0 | 2098.0 | 2095.8 | |
| I-1810 | A | C | | | 2097.0 | 2098.0 | 2095.9 | |
| I-1811 | B | | | | 2092.9 | 2095.1 | 2093.0 | |
| I-1812 | A | D | | | 2125.0 | 2126.1 | 2124.0 | |
| I-1813 | A | C | | | 2125.0 | 2126.1 | 2124.0 | |
| I-1814 | A | D | | | 2125.0 | 2126.1 | 2123.8 | |
| I-1815 | A | | ++ | | 2698.3 | 1350.2 | 1348.2 | |
| I-1816 | A | | ++ | | 2698.3 | 1350.2 | 1348.2 | |
| I-1817 | A | C | ++ | | 2754.3 | 1378.2 | 1376.2 | |
| I-1818 | A | | | | 2726.3 | 1364.3 | 1362.4 | |
| I-1819 | A | | | | 2726.3 | 1364.3 | 1362.2 | |
| I-1820 | A | B | ++ | | 2726.3 | 1364.2 | 1362.2 | |
| I-1821 | A | | ++ | | 2726.3 | 1364.2 | 1362.3 | |
| I-1822 | A | | ++ | | 2698.3 | 1350.2 | 1348.3 | |
| I-1823 | B | | ++ | | 2698.3 | 1350.6 | 1348.5 | |
| I-1824 | A | | | | 2108.9 | 2110.9 | 2109.2 | |
| I-1825 | A | | | | 2066.9 | 2068.8 | 2066.1 | |
| I-1826 | A | | | | 2094.9 | 2096.8 | 2095.1 | |
| I-1827 | A | | | | 2108.9 | 2110.9 | 2109.5 | |
| I-1828 | A | | | | 2066.9 | 2069.7 | 2066.8 | |
| I-1829 | A | | | | 2094.9 | 2096.9 | 2095.0 | |
| I-1830 | A | | | | 2108.9 | 2110.9 | 2107.7 | |
| I-1831 | A | | | | 2066.9 | 2068.8 | 2066.6 | |
| I-1832 | A | | | | 2094.9 | 2096.8 | 2094.9 | |
| I-1833 | A | | | | 2123.0 | 2124.9 | | |
| I-1834 | A | | | | 2080.9 | 2082.8 | 2070.7 | |
| I-1835 | A | | | | 2108.9 | 2110.9 | 2109.3 | |
| I-1836 | A | | | | 2135.0 | 1069.0 | 2135.0 | |
| I-1837 | B | | | | 2120.9 | 2123.2 | 2121.5 | |
| I-1838 | A | | | | 2135.0 | 1069.0 | 1066.8 | |
| I-1839 | A | | | | 2092.9 | 1048.0 | 1046.1 | |
| I-1840 | B | | | | 2120.9 | 1062.0 | 1060.1 | |
| I-1841 | A | | | | 2079.9 | 2081.0 | 2079.0 | |
| I-1842 | A | | | | 2079.9 | 2081.4 | 2079.2 | |
| I-1843 | A | | | | 2177.0 | 2178.1 | 2175.5 | |
| I-1844 | A | | | | 2177.0 | 2178.3 | 2176.0 | |
| I-1845 | A | | | | 2177.0 | 2178.0 | 2175.9 | |
| I-1846 | A | | | | 2166.9 | 2168.2 | 2165.7 | |
| I-1847 | A | | | | 2179.0 | 2180.1 | 2177.9 | |
| I-1848 | A | | | | 2179.0 | 2180.2 | 2177.8 | |
| I-1849 | A | | | | 2179.0 | 2180.1 | 2178.1 | |
| I-1850 | A | | | | 2122.0 | 2123.1 | 2120.9 | |
| I-1851 | A | | | | 2122.0 | 2123.1 | 2120.9 | |
| I-1852 | A | | | | 2107.9 | 2109.0 | 2106.9 | |
| I-1853 | A | | | | 2109.9 | 2111.0 | 2108.9 | |
| I-1854 | A | | | | 2219.0 | 2220.1 | 2218.7 | |
| I-1855 | A | | | + | 2205.0 | 2206.1 | 2203.6 | |
| I-1856 | A | | | | 2207.0 | 2208.1 | 2205.8 | |
| I-1857 | A | C | | ++ | 2005.9 | 2007.7 | 2006.2 | |
| I-1858 | A | C | | + | 2035.9 | 2037.6 | 2035.8 | |
| I-1859 | A | C | | + | 2035.9 | 2037.7 | 2036.4 | |
| I-1860 | A | C | | + | 2021.9 | 2023.8 | 2021.3 | |
| I-1861 | A | | | + | 2021.9 | 2023.6 | 2021.7 | |
| I-1862 | A | C | | + | 2021.9 | 2023.7 | 2021.9 | |
| I-1863 | A | | | + | 1991.8 | 1993.7 | 1991.9 | |
| I-1864 | A | | | +++ | 1991.8 | 1993.8 | 1992.3 | |
| I-1865 | A | | | + | 2021.9 | 2023.8 | 2021.9 | |
| I-1866 | A | | | ++ | 2021.9 | 2023.7 | 2022.3 | |
| I-1867 | A | | | | 2007.8 | 2009.6 | 2007.8 | |
| I-1868 | A | | | | 2007.8 | 2009.7 | | |
| I-1869 | A | | | + | 2007.8 | 2009.7 | 2007.9 | |
| I-1870 | A | | | + | 2007.8 | 2009.7 | 2008.2 | |
| I-1871 | A | | | | 2206.9 | | | A |
| I-1872 | A | | | | 2221.0 | 2222.1 | 2220.1 | A |
| I-1873 | A | D | | | 2262.0 | 2263.2 | 2261.4 | A |
| I-1874 | A | | | | 2205.0 | 2206.1 | 2204.1 | A |
| I-1875 | A | | | | 2283.0 | 2284.0 | 2282.1 | A |
| I-1876 | A | | | | 2163.0 | 2164.2 | 2162.6 | A |
| I-1877 | A | | | + | 2082.9 | 2084.1 | 2082.3 | A |
| I-1878 | A | | | + | 2096.9 | 2098.1 | 2096.2 | A |
| I-1879 | A | D | | +++ | 2138.0 | 2139.2 | 2136.9 | A |
| I-1880 | A | D | | + | 2081.0 | 2082.2 | 2080.4 | A |
| I-1881 | A | D | | + | 2158.9 | 1080.7 | 1078.8 | A |
| I-1882 | A | | | + | 2038.9 | 2040.0 | 2038.1 | A |
| I-1883 | A | | | | 2152.0 | 2157.8 | 2156.6 | |
| I-1884 | A | | | | 2152.0 | 2157.8 | 2156.0 | |
| I-1885 | A | | | | 2324.0 | | | A |
| I-1886 | A | | | | 2200.0 | 2202.3 | 2200.3 | A |
| I-1887 | A | | | | 2214.0 | 2216.0 | 2214.2 | A |
| I-1888 | A | | | | 2266.0 | 2068.0 | 2065.8 | A |
| I-1889 | A | | | | 2142.0 | 2144.1 | 2142.1 | A |
| I-1890 | A | | | | 2294.0 | | | A |
| I-1891 | A | | | | 2170.0 | 2171.9 | 2170.1 | A |
| I-1892 | A | | | | 2184.0 | 1093.5 | 1091.6 | A |
| I-1893 | A | | | | 2308.0 | 2310.0 | 2308.0 | A |
| I-1894 | A | | | | 2184.0 | 2186.0 | 2183.8 | A |
| I-1895 | A | | | | 2198.0 | 1100.5 | 1098.5 | A |
| I-1896 | A | | | | 2135.0 | 2136.9 | 2134.6 | |
| I-1897 | A | | | | 2092.9 | 2095.7 | 2093.8 | |
| I-1898 | A | | | | 2135.0 | 2138.2 | | |
| I-1899 | A | | | | 2092.9 | 2094.8 | | |
| I-1900 | B | | | | 2104.9 | 2106.8 | 2104.4 | |
| I-1901 | B | | | | 2147.0 | 2149.9 | | |
| I-1902 | B | | | | 2104.9 | 2107.1 | 2105.4 | |
| I-1903 | A | | | | 2147.0 | 2148.9 | 2147.1 | |
| I-1904 | A | | | | 2104.9 | 2107.2 | | |
| I-1905 | A | | | | 2147.0 | 2149.1 | 2147.3 | |
| I-1906 | A | | | | 2104.9 | 2107.8 | | |
| I-1907 | A | | | | 2066.9 | 2069.0 | 2067.4 | |
| I-1908 | A | | | | 2105.9 | 2108.2 | | |
| I-1909 | A | | | | 2122.9 | 2125.2 | 2123.1 | |
| I-1910 | A | | | | 2139.9 | 1071.9 | 1069.6 | |
| I-1911 | C | | | | 2139.9 | 1071.8 | 1070;1 | |
| I-1912 | A | | | | 2054.9 | 2056.3 | 2054.1 | |
| I-1913 | B | | | | 2094.0 | 2095.4 | 2093.3 | |
| I-1914 | A | | | | 2110.9 | 2112.4 | | |
| I-1915 | D | | | | 2127.9 | 2129.5 | 2126.9 | |
| I-1916 | B | | | | 2127.9 | 2129.8 | 2127.9 | |
| I-1917 | A | | | | 2689.2 | | | |
| I-1918 | A | | | | 2953.3 | | | |
| I-1919 | B | | | | 2885.4 | | | |
| I-1920 | B | | | | 3149.5 | | | |
| I-1921 | A | | | | 2175.0 | 2177;2 | | |
| I-1922 | A | | | | 2132.9 | 2136.1 | | |
| I-1923 | A | | | | 2092.9 | 2095.1 | 2093.1 | |
| I-1924 | A | | | | 2135.0 | 2136.9 | 2135.3 | |
| I-1925 | A | | | | 2120.9 | 1062.1 | 1060.1 | |
| I-1926 | A | | | | 2092.9 | 1048.1 | 1046.4 | |
| I-1927 | A | | | | 2241.0 | | | A |
| I-1928 | A | | | | 2229.0 | | | A |
| I-1929 | A | | | | 2235.0 | | | A |
| I-1930 | A | | | | 2269.0 | | | A |
| I-1931 | A | | | | 2257.0 | | | A |
| I-1932 | A | | | | 2263.0 | | | A |
| I-1933 | A | | | | 2340.0 | | | A |
| I-1934 | A | | | | 2346.0 | | | A |
| I-1935 | A | | | | 2109.0 | | | A |
| I-1936 | A | | | | 2149.0 | | | A |
| I-1937 | A | | | | 2123.0 | | | A |
| I-1938 | A | | | | 2095.0 | | | A |
| I-1939 | A | | | | 2219.0 | | | A |
| I-1940 | A | | | | 2192.0 | | | A |
| I-1941 | A | | | | 2232.1 | | | A |

| 1 | Description |
|---|---|
| I-608 | Ac-PL3-Asp-Leu-B5-Asp-Asp-dLys*3-Ala-Phe-GlnR*3-PyrS2-3Thi-BztA-Gln-NH2 |
| I-609 | Ac-PL3-Asp-Leu-B5-Asp-Asp-DGlnR*3-Ala-Phe-Lys*3-PyrS2-3Thi-BztA-Gln-NH2 |
| I-700 | Ac-PL3-Asp-Leu-B5-Asp-Asp-DGlnR*3-Ala-Phe-Lys*3-PyrS2-3Thi-BztA-Gln-NH2 |
| I-701 | Ac-PL3-Asp-Npg-B5-Asp-3COOHF-[ethylenediamine]GlnR-Ala-Phe-Leu-PyrS2-2F3MeF-BztA- |
| | GlnR-NH2 |
| I-702 | Ac-PL3-Asp-Npg-B5-Asp-3COOHF-[Me2ethylenediamine]GlnR-Ala-Phe-Leu-PyrS2-2F3MeF- |
| | BztA-GlnR-NH2 |
| I-703 | Ac-PL3-Asp-Npg-B5-Asp-3COOHF-[diaminopropane]GlnR-Ala-Phe-Leu-PyrS2-2F3MeF-BztA- |
| | GlnR-NH2 |
| I-704 | Ac-PL3-Asp-Npg-B5-Asp-3COOHF-[diaminopentane]GlnR-Ala-Phe-Leu-PyrS2-2F3MeF-BztA- |
| | GlnR-NH2 |
| I-705 | Ac-PL3-Asp-Npg-B5-Asp-3COOHF-[Me2diaminohexane]GlnR-Ala-Phe-Leu-PyrS2-2F3MeF- |
| | BztA-GlnR-NH2 |
| I-706 | Ac-PL3-Asp-Ile-B5-Asp-3COOHF-Aib-Ala-Phe-Lys*3-PyrS2-3Thi-BztA-GlnR*3-Ala-NH2 |
| I-707 | Ac-PL3-Asp-Chg-B5-Asp-3COOHF-Aib-Ala-Phe-Lys*3-PyrS2-3Thi-BztA-GlnR*3-Ala-NH2 |
| I-708 | Ac-PL3-Asp-DipA-B5-Asp-3COOHF-Aib-Ala-Phe-Lys*3-PyrS2-3Thi-BztA-GlnR*3-Ala-NH2 |
| I-709 | Ac-PL3-Asp-Npg-B5-Asp-3COOHF-Aib-Ala-Phe-TriAzLys*3-PyrS2-3Thi-BztA-sAla*3-Ala-NH2 |
| I-710 | Ac-PL3-Asp-Npg-B5-Asp-3COOHF-Aib-Ala-Phe-1MeK*3-PyrS2-3Thi-BztA-hGlnR*3-Ala-NH2 |
| I-711 | Ac-PL3-Asp-Npg-B5-Asp-3COOHF-Aib-Ala-Phe-1MeK*3-PyrS2-3Thi-BztA-GlnR*3-Ala-NH2 |
| I-712 | Ac-PL3-Asp-Npg-B5-Asp-3COOHF-Aib-Ala-Phe-Lys*3-PyrS2-3Thi-BztA-GlnR*3-NH2 |
| I-713 | Ac-PL3-Asp-Npg-B5-Asp-3COOHF-Aib-Ala-Phe-Lys*3-PyrS2-3Thi-BztA-GlnR*3-NH2 |
| I-714 | Ac-PL3-Asp-Npg-B5-Asp-3COOHF-Aib-Ala-Phe-Lys*3-PyrS2-3Thi-BztA-GlnR*3-Gly-NH2 |
| I-715 | Ac-PL3-Asp-Npg-B5-Asp-3COOHF-Aib-Ala-Phe-Lys*3-PyrS2-3Thi-BztA-GlnR*3-Leu-NH2 |
| I-716 | Ac-PL3-Asp-Npg-B5-Asp-3COOHF-Aib-Ala-Phe-Lys*3-PyrS2-3Thi-BztA-GlnR*3-Leu-NH2 |
| I-717 | Ac-PL3-Asp-Npg-B5-Asp-3COOHF-Aib-Ala-Phe-Lys*3-PyrS2-3Thi-BztA-GlnR*3-Npg-NH2 |
| I-718 | Ac-PL3-Asp-Npg-B5-Asp-3COOHF-Aib-Ala-Phe-Lys*3-PyrS2-3Thi-BztA-GlnR*3-Npg-NH2 |
| I-719 | Ac-PL3-Asp-Npg-B5-Asp-3COOHF-Aib-Ala-Phe-Lys*3-PyrS2-3Thi-BztA-GlnR*3-Pro-NH2 |
| I-720 | Ac-PL3-Asp-Npg-B5-Asp-3COOHF-Aib-Ala-Phe-Lys*3-PyrS2-3Thi-BztA-GlnR*3-dPro-NH2 |
| I-721 | Ac-PL3-Asp-Npg-B5-Asp-3COOHF-Aib-Ala-Phe-Lys*3-PyrS2-3Thi-BztA-GlnR*3-Ser-NH2 |
| I-722 | Ac-PL3-Asp-Npg-B5-Asp-3COOHF-Aib-Ala-Phe-Lys*3-PyrS2-3Thi-BztA-GlnR*3-Phe-NH2 |
| I-723 | Ac-PL3-Asp-Npg-B5-Asp-3COOHF-Aib-Ala-Phe-Lys*3-PyrS2-3Thi-BztA-GlnR*3-Asn-NH2 |
| I-724 | Ac-PL3-Asp-Npg-B5-Asp-3COOHF-Aib-Ala-Phe-Lys*3-PyrS2-3Thi-BztA-GlnR*3-Gln-NH2 |
| I-725 | Ac-PL3-Asp-Npg-B5-Asp-3COOHF-Aib-Ala-Phe-Lys*3-PyrS2-3Thi-BztA-GlnR*3-Trp-NH2 |
| I-726 | Ac-PL3-Asp-Npg-B5-Asp-3COOHF-Aib-Ala-Phe-Lys*3-PyrS2-3Thi-BztA-GlnR*3-Trp-NH2 |
| I-727 | Ac-PL3-Asp-Npg-B5-Asp-3COOHF-Aib-Ala-Phe-Lys*3-PyrS2-3Thi-BztA-GlnR*3-Tyr-NH2 |
| I-728 | Ac-PL3-Asp-Npg-B5-Asp-3COOHF-Aib-Ala-Phe-Lys*3-PyrS2-3Thi-BztA-GlnR*3-Tyr-NH2 |
| I-729 | Ac-PL3-Asp-Ile-B5-Asp-3COOHF-Phe-Ala-Phe-Lys*3-PyrS2-3Thi-BztA-GlnR*3-Ala-NH2 |
| I-730 | Ac-PL3-Asp-Ile-B5-Asp-3COOHF-CyLeu-Ala-Phe-Lys*3-PyrS2-3Thi-BztA-GlnR*3-Ala-NH2 |
| I-731 | Ac-PL3-Asp-Ile-B5-Asp-3COOHF-Cha-Ala-Phe-Lys*3-PyrS2-3Thi-BztA-GlnR*3-Ala-NH2 |
| I-732 | Ac-PL3-Asp-Cba-B5-Asp-3COOHF-Phe-Ala-Phe-Lys*3-PyrS2-3Thi-BztA-GlnR*3-Ala-NH2 |
| I-733 | Ac-PL3-Asp-Cba-B5-Asp-3COOHF-CyLeu-Ala-Phe-Lys*3-PyrS2-3Thi-BztA-GlnR*3-Ala-NH2 |
| I-734 | Ac-PL3-Asp-Cba-B5-Asp-3COOHF-Cha-Ala-Phe-Lys*3-PyrS2-3Thi-BztA-GlnR*3-Ala-NH2 |
| I-735 | Ac-PL3-Asp-Chg-B5-Asp-3COOHF-Phe-Ala-Phe-Lys*3-PyrS2-3Thi-BztA-GlnR*3-Ala-NH2 |
| I-736 | Ac-PL3-Asp-Chg-B5-Asp-3COOHF-CyLeu-Ala-Phe-Lys*3-PyrS2-3Thi-BztA-GlnR*3-Ala-NH2 |
| I-737 | Ac-PL3-Asp-Chg-B5-Asp-3COOHF-Cha-Ala-Phe-Lys*3-PyrS2-3Thi-BztA-GlnR*3-Ala-NH2 |
| I-738 | Ac-PL3-Asp-DipA-B5-Asp-3COOHF-Phe-Ala-Phe-Lys*3-PyrS2-3Thi-BztA-GlnR*3-Ala-NH2 |
| I-739 | Ac-PL3-Asp-DipA-B5-Asp-3COOHF-Phe-Ala-Phe-Lys*3-PyrS2-3Thi-BztA-GlnR*3-Ala-NH2 |
| I-740 | Ac-PL3-Asp-DipA-B5-Asp-3COOHF-CyLeu-Ala-Phe-Lys*3-PyrS2-3Thi-BztA-GlnR*3-Ala-NH2 |
| I-741 | Ac-PL3-3COOHF-Npg-B5-Asp-Asp-Aib-Ala-Phe-Lys*3-PyrS2-3Thi-BztA-GlnR*3-Ala-NH2 |
| I-742 | Ac-PL3-Asp-Npg-B5-3COOHF-Asp-Aib-Ala-Phe-Lys*3-PyrS2-3Thi-BztA-GlnR*3-Ala-NH2 |
| I-743 | Ac-PL3-Asp-Lys**3-B5-Asp-3COOHF-GlnR**3-Ala-Phe-Lys*3-PyrS2-3Thi-BztA-GlnR*3-Ala- |
| | NH2 |
| I-744 | Ac-PL3-Asp-1MeK**3-B5-Asp-3COOHF-GlnR**3-Ala-Phe-Lys*3-PyrS2-3Thi-BztA-GlnR*3- |
| | Ala-NH2 |
| I-745 | Ac-PL3-Asp-1MeK**3-B5-Asp-3COOHF-GlnR**3-Ala-Phe-Lys*3-PyrS2-3Thi-BztA-GlnR*3- |
| | Ala-NH2 |
| I-746 | Hex-PL3-Asp-Npg-B5-Asp-3COOHF-Aib-Ala-Phe-Lys*3-PyrS2-3Thi-BztA-GlnR*3-Ala-NH2 |
| I-747 | Bua-PL3-Asp-Npg-B5-Asp-3COOHF-Aib-Ala-Phe-Lys*3-PyrS2-3Thi-BztA-GlnR*3-Ala-NH2 |
| I-748 | 2PyzCO-PL3-Asp-Npg-B5-Asp-3COOHF-Aib-Ala-Phe-Lys*3-PyrS2-3Thi-BztA-GlnR*3-Ala-NH2 |
| I-749 | 3Phc3-PL3-Asp-Npg-B5-Asp-3COOHF-Aib-Ala-Phe-Lys*3-PyrS2-3Thi-BztA-GlnR*3-Ala-NH2 |
| I-750 | MeOPr-PL3-Asp-Npg-B5-Asp-3COOHF-Aib-Ala-Phe-Lys*3-PyrS2-3Thi-BztA-GlnR*3-Ala-NH2 |
| I-751 | lithocholate-PL3-Asp-Npg-B5-Asp-3COOHF-Aib-Ala-Phe-Lys*3-PyrS2-3Thi-BztA-GlnR*3-Ala- |
| | NH2 |
| I-752 | 2FPhc-PL3-Asp-Npg-B5-Asp-3COOHF-Aib-Ala-Phe-Lys*3-PyrS2-3Thi-BztA-GlnR*3-Ala-NH2 |
| I-753 | PhC-PL3-Asp-Npg-B5-Asp-3COOHF-Aib-Ala-Phe-Lys*3-PyrS2-3Thi-BztA-GlnR*3-Ala-NH2 |
| I-754 | MeSO2-PL3-Asp-Npg-B5-Asp-3COOHF-Aib-Ala-Phe-Lys*3-PyrS2-3Thi-BztA-GlnR*3-Ala-NH2 |
| I-755 | Ts-PL3-Asp-Npg-B5-Asp-3COOHF-Aib-Ala-Phe-Lys*3-PyrS2-3Thi-BztA-GlnR*3-Ala-NH2 |
| I-756 | Ts-PL3-Asp-Npg-B5-Asp-3COOHF-Aib-Ala-Phe-Lys*3-PyrS2-3Thi-BztA-GlnR*3-Ala-NH2 |
| I-757 | Ac-PL3-Asp-Npg-B5-Asp-3COOHF-Aib-Ala-Phe-[pXyl]hCys-PyrS2-3Thi-BztA-Cys-Ala-NH2 |
| I-758 | Ac-PL3-Asp-Npg-B5-Asp-3COOHF-Aib-Ala-Phe-[pXyl]Cys-PyrS2-3Thi-BztA-hCys-Ala-NH2 |
| I-759 | Ac-PL3-Asp-Npg-B5-Asp-3COOHF-Aib-Ala-Phe-[mXyl]hCys-PyrS2-3Thi-BztA-hCys-Ala-NH2 |
| I-760 | Ac-PL3-Asp-Npg-B5-Asp-3COOHF-Aib-Ala-Phe-[pXyl]hCys-PyrS2-3Thi-BztA-hCys-Ala-NH2 |
| I-761 | Ac-PL3-Asp-Npg-B5-Asp-3COOHF-Aib-Ala-Phe-Lys*3-PyrS2-hnLeu-34ClF-GlnR*3-Ala-NH2 |
| I-762 | Ac-PL3-Asp-Npg-B5-Asp-3COOHF-Aib-Ala-Phe-Lys*3-PyrS2-hnLeu-34ClF-GlnR*3-Ala-NH2 |
| I-763 | Ac-PL3-Asp-Npg-B5-Asp-3COOHF-Aib-Ala-Phe-Lys*3-PyrS2-hnLeu-34MeF-GlnR*3-Ala-NH2 |
| I-764 | Ac-PL3-Asp-Npg-B5-Asp-3COOHF-Aib-Ala-Phe-Lys*3-PyrS2-hnLeu-34MeF-GlnR*3-Ala-NH2 |
| I-765 | Ac-PL3-Asp-Npg-B5-Asp-3COOHF-Aib-Ala-Phe-Lys*3-PyrS2-hnLeu-3BrF-GlnR*3-Ala-NH2 |
| I-766 | Ac-PL3-Asp-Npg-B5-Asp-3COOHF-Aib-Ala-Phe-Lys*3-PyrS2-hnLeu-3BrF-GlnR*3-Ala-NH2 |
| I-767 | Ac-PL3-Asp-Npg-B5-Asp-3COOHF-Aib-Ala-Phe-Lys*3-PyrS2-hnLeu-2NapA-GlnR*3-Ala-NH2 |
| I-768 | Ac-PL3-Asp-Npg-B5-Asp-3COOHF-Aib-Ala-Phe-Lys*3-PyrS2-hnLeu-2NapA-GlnR*3-Ala-NH2 |
| I-769 | Ac-PL3-Asp-Npg-B5-Asp-3COOHF-Aib-Ala-Phe-Lys*3-PyrS2-hnLeu-RbMe2NapA-GlnR*3-Ala- |
| | NH2 |
| I-770 | Ac-PL3-Asp-Npg-B5-Asp-3COOHF-Aib-Ala-Phe-Lys*3-PyrS2-hnLeu-RbMe2NapA-GlnR*3-Ala- |
| | NH2 |
| I-771 | Ac-PL3-Asp-Npg-B5-Asp-3COOHF-Aib-Ala-Phe-Lys*3-PyrS2-hnLeu-RbMeBzta-GlnR*3-Ala- |
| | NH2 |
| I-772 | Ac-PL3-Asp-Npg-B5-Asp-3COOHF-Aib-Ala-Phe-Lys*3-PyrS2-hnLeu-RbMeBzta-GlnR*3-Ala- |
| | NH2 |
| I-773 | Ac-PL3-Asp-Npg-B5-Asp-3COOHF-Aib-Ala-Phe-Lys*3-PyrS2-hnLeu-SbMeBzta-GlnR*3-Ala- |
| | NH2 |
| I-774 | Ac-PL3-Asp-Npg-B5-Asp-3COOHF-Aib-Ala-Phe-Lys*3-PyrS2-hnLeu-SbMeBzta-GlnR*3-Ala- |
| | NH2 |
| I-775 | Ac-PL3-Asp-Npg-B5-Asp-3COOHF-Aib-Ala-Phe-Lys*3-PyrS2-hnLeu-5IndA-GlnR*3-Ala-NH2 |
| I-776 | Ac-PL3-Asp-Npg-B5-Asp-3COOHF-Aib-Ala-Phe-Lys*3-PyrS2-hnLeu-5IndA-GlnR*3-Ala-NH2 |
| I-777 | Ac-PL3-Asp-Npg-B5-Asp-3COOHF-Ala-Ala-Phe-Lys*3-SPip2-2F3MeF-BztA-GlnR*3-Ala-NH2 |
| I-778 | Ac-PL3-Asp-Npg-B5-Asp-3COOHF-Ala-Ala-Phe-Lys*3-SPip3-2F3MeF-BztA-GlnR*3-Ala-NH2 |
| I-779 | Ac-PL3-Asp-Npg-B5-Asp-3COOHF-Ala-Ala-Phe-Lys*3-SPip3-2F3MeF-BztA-GlnR*3-Ala-NH2 |
| I-780 | Ac-PL3-Asp-Npg-B5-Asp-3COOHF-Ala-Ala-Phe-Lys*3-SPip2-3Thi-BztA-GlnR*3-Ala-NH2 |
| I-781 | Ac-PL3-Asp-Npg-B5-Asp-3COOHF-Ala-Ala-Phe-Lys*3-SPip3-3Thi-BztA-GlnR*3-Ala-NH2 |
| I-782 | Ac-PL3-Asp-Npg-B5-Asp-3COOHF-Aib-Ala-Phe-Lys*3-PyrS2-3Thi-BztA-GlnR*3-Ala-Ala-Ala- |
| | NH2 |
| I-783 | Ac-PL3-Asp-Npg-B5-Asp-3COOHF-Aib-Ala-Phe-Lys*3-PyrS2-3Thi-BztA-GlnR*3-Ala-Ala-Ala- |
| | Ala-Ala-NH2 |
| I-784 | Ac-PL3-Asp-Npg-B5-Asp-3COOHF-Aib-Ala-Phe-Lys*3-PyrS2-3Thi-BztA-GlnR*3-Ala-Ala-Ala- |
| | Ala-Ala-Ala-Ala-NH2 |
| I-785 | Ac-PL3-Asp-Npg-B5-Asp-3COOHF-Aib-Ala-Phe-Lys*3-PyrS2-3Thi-BztA-GlnR*3-Leu-Ser-NH2 |
| I-786 | Ac-PL3-Asp-Npg-B5-Asp-3COOHF-Aib-Ala-Phe-Lys*3-PyrS2-3Thi-BztA-hGlnR*3-NH2 |
| I-787 | Ac-PL3-Asp-Npg-B5-Asp-3COOHF-Aib-Ala-Phe-Lys*3-PyrS2-3Thi-BztA-hGlnR*3-Ala-NH2 |
| I-788 | Ac-PL3-Asp-Npg-B5-Asp-3COOHF-Aib-Ala-Phe-dDab*3-PyrS2-3Thi-BztA-GlnR*3-NH2 |
| I-789 | Ac-PL3-Asp-Npg-B5-Asp-3COOHF-Aib-Ala-Phe-dDab*3-PyrS2-3Thi-BztA-GlnR*3-Ala-Ala- |
| | Ala-NH2 |
| I-790 | Ac-PL3-Asp-Npg-B5-Asp-3COOHF-Aib-Ala-Phe-dDab*3-PyrS2-3Thi-BztA-GlnR*3-Ala-Ala- |
| | Ala-Ala-Ala-NH2 |
| I-791 | Ac-PL3-Asp-Npg-B5-Asp-3COOHF-Aib-Ala-Phe-dDab*3-PyrS2-3Thi-BztA-GlnR*3-Leu-Ser- |
| | NH2 |
| I-792 | Ac-PL3-Asp-Npg-B5-Asp-3COOHF-Aib-Ala-Phe-dOrn*3-PyrS2-3Thi-BztA-AsnR*3-NH2 |
| I-793 | Ac-PL3-Asp-Npg-B5-Asp-3COOHF-Aib-Ala-Phe-dOrn*3-PyrS2-3Thi-BztA-AsnR*3-Ala-NH2 |
| I-794 | Ac-PL3-Asp-Npg-B5-Asp-3COOHF-Aib-Ala-Phe-dOrn*3-PyrS2-3Thi-BztA-AsnR*3-Ala-NH2 |
| I-795 | Ac-PL3-Asp-Npg-B5-Asp-3COOHF-Aib-Ala-Phe-dOrn*3-PyrS2-3Thi-BztA-AsnR*3-Ala-Ala- |
| | Ala-NH2 |
| I-796 | Ac-PL3-Asp-Npg-B5-Asp-3COOHF-Aib-Ala-Phe-dOrn*3-PyrS2-3Thi-BztA-AsnR*3-Ala-Ala- |
| | Ala-NH2 |
| I-797 | Ac-PL3-Asp-Npg-B5-Asp-3COOHF-Aib-Ala-Phe-dOrn*3-PyrS2-3Thi-BztA-AsnR*3-Leu-Ser- |
| | NH2 |
| I-798 | Ac-PL3-Asp-Npg-B5-Asp-3COOHF-Aib-Ala-Phe-dOrn*3-PyrS2-3Thi-BztA-AsnR*3-Leu-Ser- |
| | NH2 |
| I-799 | Isobutyryl-PL3-Asp-Npg-B5-Asp-3COOHF-Aib-Ala-Phe-Lys*3-PyrS2-3Thi-BztA-GlnR*3-Ala- |
| | NH2 |
| I-800 | Isobutyryl-PL3-Asp-Npg-B5-Asp-3COOHF-Aib-Ala-Phe-Lys*3-PyrS2-3Thi-BztA-GlnR*3-Ala- |
| | NH2 |
| I-801 | Isovaleryl-PL3-Asp-Npg-B5-Asp-3COOHF-Aib-Ala-Phe-Lys*3-PyrS2-3Thi-BztA-GlnR*3-Ala- |
| | NH2 |
| I-802 | EtHNCO-PL3-Asp-Npg-B5-Asp-3COOHF-Aib-Ala-Phe-Lys*3-PyrS2-3Thi-BztA-GlnR*3-Ala- |
| | NH2 |
| I-803 | Ac-PL3-Asp-Npg-B5-Asp-3COOHF-Aib-Ala_D3-Phe-Lys*3-PyrS2-3Thi-BztA-GlnR*3-Ala D3- |
| | NH2 |
| I-804 | Ac-PL3-Asp-Npg-B5-Asp-3COOHF-Aib-Ala-Phe-Lys*3-PyrS2-3Thi-BztA-GlnR*3-Ala-Ala-Leu- |
| | NH2 |
| I-805 | Ac-PL3-Asp-Npg-B5-Asp-3COOHF-Aib-Ala-Phe-Lys*3-PyrS2-3Thi-BztA-GlnR*3-Ala-Ala-Leu- |
| | NH2 |
| I-806 | Ac-PL3-Asp-Npg-B5-Asp-3COOHF-Aib-Ala-Phe-Lys*3-PyrS2-3Thi-BztA-GlnR*3-Ala-Ala-Leu- |
| | Leu-NH2 |
| I-807 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-NH2 |
| I-808 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-NH2 |
| I-809 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-NH2 |
| I-810 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-Leu-NH2 |
| I-811 | Ac-PL3-Asp-Npg-B5-Asp-3COOHF-Aib-Ala-Phe-Lys*3-PyrS2-3Thi-BztA-GlnR*3-Ala-Ala-Ala- |
| | Leu-Leu-Leu-NH2 |
| I-812 | Ac-PL3-Asp-Npg-B5-Asp-3COOHF-Aib-Ala-Phe-Lys*3-PyrS2-3Thi-BztA-GlnR*3-Ala-Val-Pro- |
| | Thr-Leu-Lys-NH2 |
| I-813 | Ac-PL3-Asp-Npg-B5-Asp-3COOHF-Aib-Ala-Phe-Lys*3-PyrS2-3Thi-BztA-GlnR*3-Ala-Ala-Val- |
| | Pro-Thr-Leu-Lys-NH2 |
| I-814 | Ac-PL3-Asp-Npg-B5-Asp-3COOHF-Aib-Ala-Phe-Lys*3-PyrS2-3Thi-BztA-GlnR*3-Ala-Lys-Leu- |
| | Pro-Val-nLeu-NH2 |
| I-815 | Ac-PL3-Asp-Npg-B5-Asp-3COOHF-Aib-Ala-Phe-Lys*3-PyrS2-3Thi-BztA-GlnR*3-Ala-Ala-Lys- |
| | Leu-Pro-Val-nLeu-NH2 |
| I-816 | Ac-PL3-Asp-Npg-B5-Asp-3COOHF-Aib-Ala-Phe-Lys*3-PyrS2-3Thi-BztA-GlnR*3-Ala-Val-Pro- |
| | Ala-Leu-Arg-NH2 |
| I-817 | Ac-PL3-Asp-Npg-B5-Asp-3COOHF-Aib-Ala-Phe-Lys*3-PyrS2-3Thi-BztA-GlnR*3-Ala-Ala-Val- |
| | Pro-Ala-Leu-Arg-NH2 |
| I-818 | 5hexenyl-MePro-Asp-[Phc][Allyl]Dap-R5-Asp-3COOHF-Aib-Ala-Phe-Lys*3-PyrS2-3Thi-BztA- |
| | GlnR*3-Ala-NH2 |
| I-819 | Ac-PL3-NAsp-Npg-B5-Asp-3COOHF-Aib-Ala-Phe-Lys*3-PyrS2-3Thi-BztA-GlnR*3-Ala-NH2 |
| I-820 | Ac-PL3-Asp-Npg-B5-Asp-3COOHF-Ala-Ala-Phe-Lys*3-PyrS2-2F3MeF-BztA-GlnR*3-Val-NH2 |
| I-821 | Ac-PL3-Asp-Npg-B5-Asp-3COOHF-Ala-Ala-Phe-Lys*3-PyrS2-2F3MeF-BztA-GlnR*3-Val-NH2 |
| I-822 | Ac-PL3-Asp-Npg-B5-Asp-3COOHF-Ala-Ala-Phe-Lys*3-PyrS2-2F3MeF-BztA-GlnR*3-Val-Ser- |
| | NH2 |
| I-823 | Ac-PL3-Asp-Npg-B5-Asp-3COOHF-Ala-Ala-Phe-Lys*3-PyrS2-2F3MeF-34ClF-GlnR*3-Ala-NH2 |
| I-824 | Ac-PL3-Asp-Npg-B5-Asp-3COOHF-Ala-Ala-Phe-Lys*3-PyrS2-2F3MeF-34ClF-GlnR*3-Val-NH2 |
| I-825 | Ac-PL3-Asp-Npg-B5-Asp-3COOHF-Ala-Ala-Phe-Lys*3-PyrS2-2F3MeF-34ClF-GlnR*3-Val-NH2 |
| I-826 | Ac-PL3-Asp-Npg-B5-Asp-3COOHF-Ala-Ala-Phe-Lys*3-PyrS2-2F3MeF-34ClF-GlnR*3-Val-Ser- |
| | NH2 |
| I-827 | Ac-PL3-Asp-Npg-B5-Asp-3COOHF-Ala-Ala-Phe-Lys*3-PyrS2-2F3MeF-34ClF-GlnR*3-Leu-NH2 |
| I-828 | Ac-PL3-Asp-Npg-B5-Asp-3COOHF-Ala-Ala-Phe-Lys*3-PyrS2-2F3MeF-34ClF-GlnR*3-Leu-NH2 |
| I-829 | Ac-PL3-Asp-Npg-B5-Asp-3COOHF-Ala-Ala-Phe-Lys*3-PyrS2-2F3MeF-34ClF-GlnR*3-Leu-Ser- |
| | NH2 |
| I-830 | Ac-PL3-Asp-Npg-B5-Asp-3COOHF-Ala-Ala-Phe-Lys*3-PyrS2-2F3MeF-34ClF-GlnR*3-Leu-Ser- |
| | NH2 |
| I-831 | Ac-PL3-Asp-Npg-B5-Asp-3COOHF-Ala-Ala-Phe-Lys*3-PyrS2-Phe-34ClF-GlnR*3-Ala-NH2 |
| I-832 | Ac-PL3-Asp-Npg-B5-Asp-3COOHF-Ala-Ala-Phe-Lys*3-PyrS2-Phe-34ClF-GlnR*3-Ala-NH2 |
| I-833 | Ac-PL3-Asp-Npg-B5-Asp-3COOHF-Ala-Ala-Phe-Lys*3-PyrS2-Phe-34ClF-GlnR*3-Val-NH2 |
| I-834 | Ac-PL3-Asp-Npg-B5-Asp-3COOHF-Ala-Ala-Phe-Lys*3-PyrS2-Phe-34ClF-GlnR*3-Val-NH2 |
| I-835 | Ac-PL3-Asp-Npg-B5-Asp-3COOHF-Ala-Ala-Phe-Lys*3-PyrS2-Phe-34ClF-GlnR*3-Val-Ser-NH2 |
| I-836 | Ac-PL3-Asp-Npg-B5-Asp-3COOHF-Ala-Ala-Phe-Lys*3-PyrS2-Phe-34ClF-GlnR*3-Val-Ser-NH2 |
| I-837 | Ac-PL3-Asp-Npg-B5-Asp-3COOHF-Ala-Ala-Phe-Lys*3-PyrS2-Phe-34ClF-GlnR*3-Leu-NH2 |
| I-838 | Ac-PL3-Asp-Npg-B5-Asp-3COOHF-Ala-Ala-Phe-Lys*3-PyrS2-Phe-34ClF-GlnR*3-Leu-NH2 |
| I-839 | Ac-PL3-Asp-Npg-B5-Asp-3COOHF-Ala-Ala-Phe-Lys*3-PyrS2-Phe-34ClF-GlnR*3-Leu-Ser-NH2 |
| I-840 | Ac-PL3-Asp-Npg-B5-Asp-3COOHF-Ala-Ala-Phe-Lys*3-PyrS2-Phe-34ClF-GlnR*3-Leu-Ser-NH2 |
| I-841 | Ac-PL3-Asp-Cha-B5-Asp-3COOHF-Phe-Ala-Phe-Lys*3-PyrS2-3Thi-BztA-GlnR*3-Ala-NH2 |
| I-842 | Ac-PL3-Asp-nLeu-B5-Asp-3COOHF-Phe-Ala-Phe-Lys*3-PyrS2-3Thi-BztA-GlnR*3-Ala-NH2 |
| I-843 | Ac-PL3-Asp-Ile-B5-Asp-3COOHF-Aic-Ala-Phe-Lys*3-PyrS2-3Thi-BztA-GlnR*3-Ala-NH2 |
| I-844 | Ac-PL3-Asp-Chg-B5-Asp-3COOHF-Aic-Ala-Phe-Lys*3-PyrS2-3Thi-BztA-GlnR*3-Ala-NH2 |
| I-845 | Ac-PL3-Asp-DipA-B5-Asp-3COOHF-Aic-Ala-Phe-Lys*3-PyrS2-3Thi-BztA-GlnR*3-Ala-NH2 |
| I-846 | Ac-PL3-Asp-Cha-B5-Asp-3COOHF-Aic-Ala-Phe-Lys*3-PyrS2-3Thi-BztA-GlnR*3-Ala-NH2 |
| I-847 | Ac-PL3-Asp-Cba-B5-Asp-3COOHF-Aic-Ala-Phe-Lys*3-PyrS2-3Thi-BztA-GlnR*3-Ala-NH2 |
| I-848 | Ac-PL3-Asp-nLeu-B5-Asp-3COOHF-Aic-Ala-Phe-Lys*3-PyrS2-3Thi-BztA-GlnR*3-Ala-NH2 |
| I-849 | Ac-PL3-Asp-Ile-B5-Asp-3COOHF-CyhLeu-Ala-Phe-Lys*3-PyrS2-3Thi-BztA-GlnR*3-Ala-NH2 |
| I-850 | Ac-PL3-Asp-Chg-B5-Asp-3COOHF-CyhLeu-Ala-Phe-Lys*3-PyrS2-3Thi-BztA-GlnR*3-Ala-NH2 |
| I-851 | Ac-PL3-Asp-DipA-B5-Asp-3COOHF-CyhLeu-Ala-Phe-Lys*3-PyrS2-3Thi-BztA-GlnR*3-Ala-NH2 |
| I-852 | Ac-PL3-Asp-Cha-B5-Asp-3COOHF-CyhLeu-Ala-Phe-Lys*3-PyrS2-3Thi-BztA-GlnR*3-Ala-NH2 |
| I-853 | Ac-PL3-Asp-Cha-B5-Asp-3COOHF-CyhLeu-Ala-Phe-Lys*3-PyrS2-3Thi-BztA-GlnR*3-Ala-NH2 |
| I-854 | Ac-PL3-Asp-Cba-B5-Asp-3COOHF-CyhLeu-Ala-Phe-Lys*3-PyrS2-3Thi-BztA-GlnR*3-Ala-NH2 |
| I-855 | Ac-PL3-Asp-nLeu-B5-Asp-3COOHF-CyhLeu-Ala-Phe-Lys*3-PyrS2-3Thi-BztA-GlnR*3-Ala-NH2 |
| I-856 | Ac-PL3-Asp-Ile-B5-Asp-3COOHF-Cbg-Ala-Phe-Lys*3-PyrS2-3Thi-BztA-GlnR*3-Ala-NH2 |
| I-857 | Ac-PL3-Asp-Chg-B5-Asp-3COOHF-Cbg-Ala-Phe-Lys*3-PyrS2-3Thi-BztA-GlnR*3-Ala-NH2 |
| I-858 | Ac-PL3-Asp-DipA-B5-Asp-3COOHF-Cbg-Ala-Phe-Lys*3-PyrS2-3Thi-BztA-GlnR*3-Ala-NH2 |
| I-859 | Ac-PL3-Asp-Cha-B5-Asp-3COOHF-Cbg-Ala-Phe-Lys*3-PyrS2-3Thi-BztA-GlnR*3-Ala-NH2 |
| I-860 | Ac-PL3-Asp-Cba-B5-Asp-3COOHF-Cbg-Ala-Phe-Lys*3-PyrS2-3Thi-BztA-GlnR*3-Ala-NH2 |
| I-861 | Ac-PL3-Asp-nLeu-B5-Asp-3COOHF-Cbg-Ala-Phe-Lys*3-PyrS2-3Thi-BztA-GlnR*3-Ala-NH2 |
| I-862 | Ac-PL3-Asp-Npg-B5-Asp-3COOHF-Aib-Ala-Phe-Lys*3-PyrS2-2F3MeF-34ClF-GlnR*3-NH2 |
| I-863 | Ac-PL3-Asp-Npg-B5-Asp-3COOHF-Aib-Ala-Phe-Lys*3-PyrS2-2F3MeF-34ClF-GlnR*3-NH2 |
| I-864 | Ac-PL3-Asp-Npg-B5-Asp-3COOHF-Aib-Ala-Phe-Lys*3-PyrS2-2F3MeF-34ClF-GlnR*3-Pro-NH2 |
| I-865 | Ac-PL3-Asp-Npg-B5-Asp-3COOHF-Aib-Ala-Phe-Lys*3-PyrS2-2F3MeF-34ClF-GlnR*3-Ser-NH2 |
| I-866 | Ac-PL3-Asp-Npg-B5-Asp-3COOHF-Aib-Ala-Phe-Lys*3-PyrS2-2F3MeF-34ClF-GlnR*3-Phe-NH2 |
| I-867 | Ac-PL3-Asp-Npg-B5-Asp-3COOHF-Aib-Ala-Phe-Lys*3-PyrS2-3Thi-34ClF-GlnR*3-NH2 |
| I-868 | Ac-PL3-Asp-Npg-B5-Asp-3COOHF-Aib-Ala-Phe-Lys*3-PyrS2-3Thi-34ClF-GlnR*3-Pro-NH2 |
| I-869 | Ac-PL3-Asp-Npg-B5-Asp-3COOHF-Aib-Ala-Phe-Lys*3-PyrS2-3Thi-34ClF-GlnR*3-Ser-NH2 |
| I-870 | Ac-PL3-Asp-Npg-B5-Asp-3COOHF-Aib-Ala-Phe-Lys*3-PyrS2-3Thi-34ClF-GlnR*3-Phe-NH2 |
| I-871 | Ac-PL3-Asp-Npg-B5-Asp-3COOHF-Aib-Ala-Phe-Lys*3-PyrS2-Phe-34ClF-GlnR*3-NH2 |
| I-872 | Ac-PL3-Asp-Npg-B5-Asp-3COOHF-Aib-Ala-Phe-Lys*3-PyrS2-Phe-34ClF-GlnR*3-Pro-NH2 |
| I-873 | Ac-PL3-Asp-Npg-B5-Asp-3COOHF-Aib-Ala-Phe-Lys*3-PyrS2-Phe-34ClF-GlnR*3-Ser-NH2 |
| I-874 | Ac-PL3-Asp-Npg-B5-Asp-3COOHF-Aib-Ala-Phe-Lys*3-PyrS2-Phe-34ClF-GlnR*3-Phe-NH2 |
| I-875 | Ac-PL3-Asp-Npg-B5-Asp-TfeGA-Aib-Ala-Phe-Lys*3-PyrS2-3Thi-BztA-GlnR*3-NH2 |
| I-876 | Ac-PL3-Asp-Npg-B5-Asp-TfeGA-Aib-Ala-Phe-Lys*3-PyrS2-3Thi-BztA-GlnR*3-Ala-NH2 |
| I-877 | Ac-PL3-Asp-Npg-B5-Asp-TfeGA-Aib-Ala-Phe-Lys*3-PyrS2-3Thi-BztA-GlnR*3-Leu-Ser-NH2 |
| I-878 | Ac-PL3-Asp-Npg-B5-Asp-TfeGA-Aib-Ala-Phe-Lys*3-PyrS2-Phe-BztA-GlnR*3-NH2 |
| I-879 | Ac-PL3-Asp-Npg-B5-Asp-TfeGA-Aib-Ala-Phe-Lys*3-PyrS2-Phe-BztA-GlnR*3-Ala-NH2 |
| I-880 | Ac-PL3-Asp-Npg-B5-Asp-TfeGA-Aib-Ala-Phe-Lys*3-PyrS2-Phe-BztA-GlnR*3-Leu-Ser-NH2 |
| I-881 | Ac-PL3-Asp-Npg-B5-Asp-TfeGA-Aib-Ala-Phe-Lys*3-PyrS2-3Thi-34ClF-GlnR*3-NH2 |
| I-882 | Ac-PL3-Asp-Npg-B5-Asp-TfeGA-Aib-Ala-Phe-Lys*3-PyrS2-3Thi-34ClF-GlnR*3-Ala-NH2 |
| I-883 | Ac-PL3-Asp-Npg-B5-Asp-TfeGA-Aib-Ala-Phe-Lys*3-PyrS2-3Thi-34ClF-GlnR*3-Leu-Ser-NH2 |
| I-884 | Ac-PL3-Asp-Npg-B5-Asp-TfeGA-Aib-Ala-Phe-Lys*3-PyrS2-Phe-34ClF-GlnR*3-NH2 |
| I-885 | Ac-PL3-Asp-Npg-B5-Asp-TfeGA-Aib-Ala-Phe-Lys*3-PyrS2-Phe-34ClF-GlnR*3-Ala-NH2 |
| I-886 | Ac-PL3-Asp-Npg-B5-Asp-TfeGA-Aib-Ala-Phe-Lys*3-PyrS2-Phe-34ClF-GlnR*3-Leu-Ser-NH2 |
| I-887 | Ac-PL3-Asp-Npg-B5-Asp-TfeGA-Aib-Ala-Phe-Lys*3-PyrS2-2F3MeF-BztA-GlnR*3-NH2 |
| I-888 | Ac-PL3-Asp-Npg-B5-Asp-TfeGA-Aib-Ala-Phe-Lys*3-PyrS2-2F3MeF-BztA-GlnR*3-Leu-Ser- |
| | NH2 |
| I-889 | Ac-PL3-Asp-Npg-B5-Asp-TfeGA-Aib-Ala-Phe-Lys*3-PyrS2-2F3MeF-BztA-GlnR*3-Leu-Ser- |
| | NH2 |
| I-890 | Ac-PL3-Asp-Npg-B5-Asp-TfeGA-Aib-Ala-Phe-Lys*3-PyrS2-2F3MeF-34ClF-GlnR*3-NH2 |
| I-891 | Ac-PL3-Asp-Npg-B5-Asp-TfeGA-Aib-Ala-Phe-Lys*3-PyrS2-2F3MeF-34ClF-GlnR*3-NH2 |
| I-892 | Ac-PL3-Asp-Npg-B5-Asp-TfeGA-Aib-Ala-Phe-Lys*3-PyrS2-2F3MeF-34ClF-GlnR*3-Ala-NH2 |
| I-893 | Ac-PL3-Asp-Npg-B5-Asp-TfeGA-Aib-Ala-Phe-Lys*3-PyrS2-2F3MeF-34ClF-GlnR*3-Leu-Ser- |
| | NH2 |
| I-894 | Ac-PL3-Asp-Npg-B5-Asp-TfeGA-Ala-Ala-Phe-Lys*3-PyrS2-Phe-BztA-GlnR*3-NH2 |
| I-895 | Ac-PL3-Asp-Npg-B5-Asp-TfeGA-Ala-Ala-Phe-Lys*3-PyrS2-Phe-BztA-GlnR*3-NH2 |
| I-896 | Ac-PL3-Asp-Npg-B5-Asp-TfeGA-Ala-Ala-Phe-Lys*3-PyrS2-Phe-BztA-GlnR*3-Ala-NH2 |
| I-897 | Ac-PL3-Asp-Npg-B5-Asp-TfeGA-Ala-Ala-Phe-Lys*3-PyrS2-Phe-BztA-GlnR*3-Ala-NH2 |
| I-898 | Ac-PL3-Asp-Npg-B5-Asp-TfeGA-Ala-Ala-Phe-Lys*3-PyrS2-Phe-BztA-GlnR*3-Leu-Ser-NH2 |
| I-899 | Ac-PL3-Asp-Npg-B5-Asp-TfeGA-Ala-Ala-Phe-Lys*3-PyrS2-Phe-BztA-GlnR*3-Leu-Ser-NH2 |
| I-900 | Ac-PL3-Asp-Npg-B5-Asp-TfeGA-Phe-Ala-Phe-Lys*3-PyrS2-Phe-BztA-GlnR*3-NH2 |
| I-901 | Ac-PL3-Asp-Npg-B5-Asp-TfeGA-Phe-Ala-Phe-Lys*3-PyrS2-Phe-BztA-GlnR*3-Ala-NH2 |
| I-902 | Ac-PL3-Asp-Npg-B5-Asp-TfeGA-Phe-Ala-Phe-Lys*3-PyrS2-Phe-BztA-GlnR*3-Leu-Ser-NH2 |
| I-903 | TzPyr-PL3-Asp-Npg-B5-Asp-3COOHF-Aib-Ala-Phe-Lys*3-PyrS2-3Thi-BztA-GlnR*3-Ala-NH2 |
| I-904 | 15PyraPy-PL3-Asp-Npg-B5-Asp-3COOHF-Aib-Ala-Phe-Lys*3-PyrS2-3Thi-BztA-GlnR*3-Ala- |
| | NH2 |
| I-905 | 15PyraPy-PL3-Asp-Npg-B5-Asp-3COOHF-Aib-Ala-Phe-Lys*3-PyrS2-3Thi-BztA-GlnR*3-Ala- |
| | NH2 |
| I-906 | 8IAP-PL3-Asp-Npg-B5-Asp-3COOHF-Aib-Ala-Phe-Lys*3-PyrS2-3Thi-BztA-GlnR*3-Ala-NH2 |
| I-907 | 3PydCO-PL3-Asp-Npg-B5-Asp-3COOHF-Aib-Ala-Phe-Lys*3-PyrS2-3Thi-BztA-GlnR*3-Ala- |
| | NH2 |
| I-908 | 2PyBu-PL3-Asp-Npg-B5-Asp-3COOHF-Aib-Ala-Phe-Lys*3-PyrS2-3Thi-BztA-GlnR*3-Ala-NH2 |
| I-909 | 2PymCO-PL3-Asp-Npg-B5-Asp-3COOHF-Aib-Ala-Phe-Lys*3-PyrS2-3Thi-BztA-GlnR*3-Ala- |
| | NH2 |
| I-910 | 5PymCO-PL3-Asp-Npg-B5-Asp-3COOHF-Aib-Ala-Phe-Lys*3-PyrS2-3Thi-BztA-GlnR*3-Ala- |
| | NH2 |
| I-911 | 4PymCO-PL3-Asp-Npg-B5-Asp-3COOHF-Aib-Ala-Phe-Lys*3-PyrS2-3Thi-BztA-GlnR*3-Ala- |
| | NH2 |
| I-912 | 4PymCO-PL3-Asp-Npg-B5-Asp-3COOHF-Aib-Ala-Phe-Lys*3-PyrS2-3Thi-BztA-GlnR*3-Ala- |
| | NH2 |
| I-913 | Ac-PL3-Asp-Npg-B5-Asp-3COOHF-Aib-Ala-Phe-Lys*3-PyrS2-2Thi-34ClF-GlnR*3-Ala-NH2 |
| I-914 | Ac-PL3-Asp-Npg-B5-Asp-3COOHF-Aib-Ala-Phe-Lys*3-PyrS2-2Thi-34ClF-GlnR*3-Ala-NH2 |
| I-915 | Ac-PL3-Asp-Npg-B5-Asp-3COOHF-Aib-Ala-Phe-Lys*3-PyrS2-2Thi-34ClF-GlnR*3-Leu-Ser-NH2 |
| I-916 | Ac-PL3-Asp-Npg-B5-Asp-3COOHF-Aib-Ala-Phe-Lys*3-PyrS2-2Thi-34ClF-GlnR*3-Leu-Ser-NH2 |
| I-917 | Ac-PL3-Asp-Npg-B5-Asp-3COOHF-Leu-Ala-Phe-Lys*3-PyrS2-2Thi-34ClF-GlnR*3-Ala-NH2 |
| I-918 | Ac-PL3-Asp-Npg-B5-Asp-3COOHF-Leu-Ala-Phe-Lys*3-PyrS2-2Thi-34ClF-GlnR*3-Ala-NH2 |
| I-919 | Ac-PL3-Asp-Npg-B5-Asp-3COOHF-Phe-Ala-Phe-Lys*3-PyrS2-2Thi-34ClF-GlnR*3-Ala-NH2 |
| I-920 | Ac-PL3-Asp-Npg-B5-Asp-3COOHF-Phe-Ala-Phe-Lys*3-PyrS2-2Thi-34ClF-GlnR*3-Ala-NH2 |
| I-921 | Ac-PL3-Asp-Npg-B5-Asp-3COOHF-Aib-Ala-2Thi-Lys*3-PyrS2-2Thi-34ClF-GlnR*3-Ala-NH2 |
| I-922 | Ac-PL3-Asp-Npg-B5-Asp-3COOHF-Aib-Ala-2Thi-Lys*3-PyrS2-2Thi-34ClF-GlnR*3-Ala-NH2 |
| I-923 | Ac-PL3-Asp-Npg-B5-Asp-3COOHF-Aib-Ala-Phe-Lys*3-PyrS2-2Thi-BztA-GlnR*3-NH2 |
| I-924 | Ac-PL3-Asp-Npg-B5-Asp-3COOHF-Aib-Ala-Phe-Lys*3-PyrS2-2Thi-BztA-GlnR*3-NH2 |
| I-925 | Ac-PL3-Asp-Npg-B5-Asp-3COOHF-Aib-Ala-Phe-Lys*3-PyrS2-2Thi-BztA-GlnR*3-Leu-Ser-NH2 |
| I-926 | Ac-PL3-Asp-Npg-B5-Asp-3COOHF-Leu-Ala-Phe-Lys*3-PyrS2-2Thi-BztA-GlnR*3-Ala-NH2 |
| I-927 | Ac-PL3-Asp-Npg-B5-Asp-3COOHF-Phe-Ala-Phe-Lys*3-PyrS2-2Thi-BztA-GlnR*3-Ala-NH2 |
| I-928 | Ac-PL3-Phe-Npg-B5-Asp-3COOHF-Aib-Ala-Phe-Lys*3-PyrS2-3Thi-BztA-GlnR*3-Ala-NH2 |
| I-929 | Ac-PL3-Gln-Npg-B5-Asp-3COOHF-Aib-Ala-Phe-Lys*3-PyrS2-3Thi-BztA-GlnR*3-Ala-NH2 |
| I-930 | Ac-PL3-Gln-Npg-B5-Asp-3COOHF-Aib-Ala-Phe-Lys*3-PyrS2-3Thi-BztA-GlnR*3-Ala-NH2 |
| I-931 | Ac-PL3-3Thi-Npg-B5-Asp-3COOHF-Aib-Ala-Phe-Lys*3-PyrS2-3Thi-BztA-GlnR*3-Ala-NH2 |
| I-932 | 4pentenyl-MePro-Asp-B5-Ala-Asp-3COOHF-Ala-Ala-Phe-PyrS2-Lys*3-3Thi-BztA-Ala-GlnR*3- |
| | NH2 |
| I-933 | 4pentenyl-ThioPro-Asp-B5-Ala-Asp-3COOHF-Ala-Ala-Phe-PyrS2-Lys*3-3Thi-BztA-Ala-GlnR*3- |
| | NH2 |
| I-934 | 4pentenyl-ThioPro-Asp-B5-Ala-Asp-3COOHF-Ala-Ala-Phe-PyrS2-Lys*3-3Thi-BztA-Ala-GlnR*3- |
| | NH2 |
| I-935 | Ac-PL3-NGlu-Npg-B5-Asp-3COOHF-Aib-Ala-Phe-Lys*3-PyrS2-3Thi-BztA-GlnR*3-Ala-NH2 |
| I-936 | Ac-PL3-Asp-Npg-B5-Asp-3COOHF-Aib-Ala-Phe-Lys*3-PyrS2-HexG-BztA-GlnR*3-Ala-NH2 |
| I-937 | Ac-PL3-Asp-Npg-B5-Asp-3COOHF-Aib-Ala-Phe-Lys*3-PyrS2-HexG-BztA-GlnR*3-Ala-NH2 |
| I-938 | Ac-PL3-Asp-Npg-B5-Asp-3COOHF-Aib-Ala-Phe-Lys*3-PyrS2-HepG-BztA-GlnR*3-Ala-NH2 |
| I-939 | Ac-PL3-Asp-Npg-B5-Asp-3COOHF-Aib-Ala-Phe-Lys*3-PyrS2-HepG-BztA-GlnR*3-Ala-NH2 |
| I-940 | Ac-PL3-Asp-Npg-B5-Asp-3COOHF-Aib-Ala-Phe-Lys*3-PyrS2-BztA-BztA-GlnR*3-Ala-NH2 |
| I-941 | Ac-PL3-Asp-Npg-B5-Asp-3COOHF-Aib-Ala-Phe-Lys*3-PyrS2-BztA-BztA-GlnR*3-Ala-NH2 |
| I-942 | Ac-PL3-Asp-Npg-B5-Asp-3COOHF-Aib-Ala-Phe-Lys*3-PyrS2-HexG-34ClF-GlnR*3-Ala-NH2 |
| I-943 | Ac-PL3-Asp-Npg-B5-Asp-3COOHF-Aib-Ala-Phe-Lys*3-PyrS2-HepG-34ClF-GlnR*3-Ala-NH2 |
| I-944 | Ac-PL3-Asp-Npg-B5-Asp-3COOHF-Aib-Ala-Phe-Lys*3-PyrS2-HepG-34ClF-GlnR*3-Ala-NH2 |
| I-945 | Ac-PL3-Asp-Npg-B5-Asp-3COOHF-Aib-Ala-Phe-Lys*3-PyrS2-BztA-34ClF-GlnR*3-Ala-NH2 |
| I-946 | Ac-PL3-Asp-Npg-B5-Asp-3COOHF-Aib-Ala-Phe-Lys*3-Az3-2F3MeF-BztA-GlnR*3-Ala-NH2 |
| I-947 | Ac-PL3-Asp-Npg-B5-Asp-3COOHF-Aib-Ala-Phe-Lys*3-Az2-3Thi-BztA-GlnR*3-Ala-NH2 |
| I-948 | Ac-PL3-Asp-Npg-B5-Asp-3COOHF-Aib-Ala-Phe-Lys*3-Az3-3Thi-BztA-GlnR*3-Ala-NH2 |
| I-949 | Ac-PL3-Asp-Npg-B5-Asp-3COOHF-Aib-Ala-Phe-Lys*3-PyrS2-2PhF-BztA-GlnR*3-Ala-NH2 |
| I-950 | Ac-PL3-Asp-Npg-B5-Asp-3COOHF-Aib-Ala-Phe-Lys*3-PyrS2-2PhF-34ClF-GlnR*3-Ala-NH2 |
| I-951 | Ac-PL3-Asp-Npg-B5-Asp-3COOHF-Aib-Ala-Phe-Lys*3-PyrS2-2PhF-34ClF-GlnR*3-Ala-NH2 |
| I-952 | Ac-PL3-Asp-Npg-B5-Asp-3COOHF-Aib-Ala-Phe-Lys*3-PyrS2-2PhF-BztA-GlnR*3-Leu-Ser-NH2 |
| I-953 | Ac-PL3-Asp-Npg-B5-Asp-3COOHF-Aib-Ala-Phe-Lys*3-PyrS2-2PhF-BztA-GlnR*3-Leu-Ser-NH2 |
| I-954 | Ac-PL3-Asp-Npg-B5-Asp-3COOHF-Aib-Ala-Phe-Lys*3-PyrS2-2PhF-34ClF-GlnR*3-Leu-Ser- |
| | NH2 |
| I-955 | Ac-PL3-Asp-Npg-B5-Asp-3COOHF-Aib-Ala-Phe-Lys*3-PyrS2-hnLeu-BztA-GlnR*3-Leu-Ser- |
| | NH2 |
| I-956 | Ac-PL3-Asp-Npg-B5-Asp-3COOHF-Aib-Ala-Phe-Lys*3-PyrS2-hnLeu-34ClF-GlnR*3-Leu-Ser- |
| | NH2 |
| I-957 | Ac-PL3-isoDAsp-Npg-B5-Asp-3COOHF-Aib-Ala-Phe-Lys*3-PyrS2-3Thi-BztA-GlnR*3-Ala-NH2 |
| I-958 | Ac-PL3-isoGlu-Npg-B5-Asp-3COOHF-Aib-Ala-Phe-Lys*3-PyrS2-3Thi-BztA-GlnR*3-Ala-NH2 |
| I-959 | Ac-PL3-isoGlu-Npg-B5-Asp-3COOHF-Aib-Ala-Phe-Lys*3-PyrS2-3Thi-BztA-GlnR*3-Ala-NH2 |
| I-960 | Ac-PL3-isoDGlu-Npg-B5-Asp-3COOHF-Aib-Ala-Phe-Lys*3-PyrS2-3Thi-BztA-GlnR*3-Ala-NH2 |
| I-961 | Ac-PL3-RbGlu-Npg-B5-Asp-3COOHF-Aib-Ala-Phe-Lys*3-PyrS2-3Thi-BztA-GlnR*3-Ala-NH2 |
| I-962 | Ac-PL3-SbGlu-Npg-B5-Asp-3COOHF-Aib-Ala-Phe-Lys*3-PyrS2-3Thi-BztA-GlnR*3-Ala-NH2 |
| I-963 | Ac-PL3-isoDAsp-Npg-B5-Asp-3COOHF-Aib-Ala-Phe-Lys*3-PyrS2-3Thi-BztA-GlnR*3-Ala-NH2 |
| I-964 | Ac-PL3-isoGlu-Npg-B5-Asp-3COOHF-Aib-Ala-Phe-Lys*3-PyrS2-3Thi-BztA-GlnR*3-Ala-NH2 |
| I-965 | Ac-PL3-RbGlu-Npg-B5-Asp-3COOHF-Aib-Ala-Phe-Lys*3-PyrS2-3Thi-BztA-GlnR*3-Ala-NH2 |
| I-966 | Ac-PL3-SbGlu-Npg-B5-Asp-3COOHF-Aib-Ala-Phe-Lys*3-PyrS2-3Thi-BztA-GlnR*3-Ala-NH2 |
| I-967 | Ac-PL3-Glu-Npg-B5-Asp-3COOHF-Aib-Ala-Phe-Lys*3-PyrS2-3Thi-BztA-GlnR*3-Ala-NH2 |
| I-968 | Ac-PL3-Gln-Npg-B5-Asp-3COOHF-Aib-Ala-Phe-Lys*3-PyrS2-3Thi-BztA-GlnR*3-Ala-NH2 |
| I-969 | Ac-PL3-Asp-Ile-B5-Asp-3COOHF-Ala-Ala-Phe-Lys*3-PyrS2-3Thi-BztA-GlnR*3-Ala-NH2 |
| I-970 | Ac-PL3-Asp-Ile-B5-Asp-3COOHF-Ala-Ala-Phe-Lys*3-PyrS2-3Thi-BztA-GlnR*3-Ala-NH2 |
| I-971 | Ac-PL3-Asp-Ile-B5-Asp-3COOHF-Val-Ala-Phe-Lys*3-PyrS2-3Thi-BztA-GlnR*3-Ala-NH2 |
| I-972 | Ac-PL3-Asp-Ile-B5-Asp-3COOHF-Leu-Ala-Phe-Lys*3-PyrS2-3Thi-BztA-GlnR*3-Ala-NH2 |
| I-973 | Ac-PL3-Asp-Ile-B5-Asp-3COOHF-nLeu-Ala-Phe-Lys*3-PyrS2-3Thi-BztA-GlnR*3-Ala-NH2 |
| I-974 | Ac-PL3-Asp-Ile-B5-Asp-3COOHF-nLeu-Ala-Phe-Lys*3-PyrS2-3Thi-BztA-GlnR*3-Ala-NH2 |
| I-975 | Ac-PL3-Asp-Ile-B5-Asp-3COOHF-Dpg-Ala-Phe-Lys*3-PyrS2-3Thi-BztA-GlnR*3-Ala-NH2 |
| I-976 | Ac-PL3-Asp-Chg-B5-Asp-3COOHF-Ala-Ala-Phe-Lys*3-PyrS2-3Thi-BztA-GlnR*3-Ala-NH2 |
| I-977 | Ac-PL3-Asp-Chg-B5-Asp-3COOHF-Ala-Ala-Phe-Lys*3-PyrS2-3Thi-BztA-GlnR*3-Ala-NH2 |
| I-978 | Ac-PL3-Asp-Chg-B5-Asp-3COOHF-Val-Ala-Phe-Lys*3-PyrS2-3Thi-BztA-GlnR*3-Ala-NH2 |
| I-979 | Ac-PL3-Asp-Chg-B5-Asp-3COOHF-nLeu-Ala-Phe-Lys*3-PyrS2-3Thi-BztA-GlnR*3-Ala-NH2 |
| I-980 | Ac-PL3-Asp-Chg-B5-Asp-3COOHF-Dpg-Ala-Phe-Lys*3-PyrS2-3Thi-BztA-GlnR*3-Ala-NH2 |
| I-981 | Ac-PL3-Asp-Chg-B5-Asp-3COOHF-Dpg-Ala-Phe-Lys*3-PyrS2-3Thi-BztA-GlnR*3-Ala-NH2 |
| I-982 | Ac-PL3-Asp-DipA-B5-Asp-3COOHF-Ala-Ala-Phe-Lys*3-PyrS2-3Thi-BztA-GlnR*3-Ala-NH2 |
| I-983 | Ac-PL3-Asp-DipA-B5-Asp-3COOHF-Ala-Ala-Phe-Lys*3-PyrS2-3Thi-BztA-GlnR*3-Ala-NH2 |
| I-984 | Ac-PL3-Asp-DipA-B5-Asp-3COOHF-Val-Ala-Phe-Lys*3-PyrS2-3Thi-BztA-GlnR*3-Ala-NH2 |
| I-985 | Ac-PL3-Asp-DipA-B5-Asp-3COOHF-Leu-Ala-Phe-Lys*3-PyrS2-3Thi-BztA-GlnR*3-Ala-NH2 |
| I-986 | Ac-PL3-Asp-DipA-B5-Asp-3COOHF-nLeu-Ala-Phe-Lys*3-PyrS2-3Thi-BztA-GlnR*3-Ala-NH2 |
| I-987 | Ac-PL3-Asp-Cba-B5-Asp-3COOHF-Ala-Ala-Phe-Lys*3-PyrS2-3Thi-BztA-GlnR*3-Ala-NH2 |
| I-988 | Ac-PL3-Asp-Cba-B5-Asp-3COOHF-Val-Ala-Phe-Lys*3-PyrS2-3Thi-BztA-GlnR*3-Ala-NH2 |
| I-989 | Ac-PL3-Asp-Cba-B5-Asp-3COOHF-Leu-Ala-Phe-Lys*3-PyrS2-3Thi-BztA-GlnR*3-Ala-NH2 |
| I-990 | Ac-PL3-Asp-Cba-B5-Asp-3COOHF-nLeu-Ala-Phe-Lys*3-PyrS2-3Thi-BztA-GlnR*3-Ala-NH2 |
| I-991 | Ac-PL3-Asp-Cba-B5-Asp-3COOHF-Dpg-Ala-Phe-Lys*3-PyrS2-3Thi-BztA-GlnR*3-Ala-NH2 |
| I-992 | 5hexenyl-MePro-Asp-B5-Ala-Asp-3COOHF-Ala-Ala-Phe-PyrS2-Lys*3-3Thi-BztA-Ala-GlnR*3- |
| | NH2 |
| I-993 | 4pentenyl-MePro-Asp-B5-Ala-Asp-3COOHF-Ala-Ala-Phe-PyrS2-Lys*3-3Thi-BztA-Ala-GlnR*3- |
| | NH2 |
| I-994 | Ac-PL3-Asp-Chg-B5-Asp-3COOHF-Gly-Ala-Phe-Lys*3-PyrS2-3Thi-BztA-GlnR*3-Ala-NH2 |
| I-995 | Ac-PL3-Asp-Chg-B5-Asp-3COOHF-Gly-Ala-Phe-Lys*3-PyrS2-3Thi-BztA-GlnR*3-Ala-NH2 |
| I-996 | Ac-PL3-Asp-Chg-B5-Asp-3COOHF-dAla-Ala-Phe-Lys*3-PyrS2-3Thi-BztA-GlnR*3-Ala-NH2 |
| I-997 | Ac-PL3-Asp-Chg-B5-Asp-3COOHF-Aib-Thr-Phe-Lys*3-PyrS2-3Thi-BztA-GlnR*3-Ala-NH2 |
| I-998 | Ac-PL3-Asp-Chg-B5-Asp-3COOHF-Aib-aThr-Phe-Lys*3-PyrS2-3Thi-BztA-GlnR*3-Ala-NH2 |
| I-999 | Ac-PL3-Asp-Chg-B5-Asp-3COOHF-Aib-Gly-Phe-Lys*3-PyrS2-3Thi-BztA-GlnR*3-Ala-NH2 |
| I-1000 | Ac-PL3-Asp-DipA-B5-Asp-3COOHF-Gly-Ala-Phe-Lys*3-PyrS2-3Thi-BztA-GlnR*3-Ala-NH2 |
| I-1001 | Ac-PL3-Asp-DipA-B5-Asp-3COOHF-Gly-Ala-Phe-Lys*3-PyrS2-3Thi-BztA-GlnR*3-Ala-NH2 |
| I-1002 | Ac-PL3-Asp-DipA-B5-Asp-3COOHF-dAla-Ala-Phe-Lys*3-PyrS2-3Thi-BztA-GlnR*3-Ala-NH2 |
| I-1003 | Ac-PL3-Asp-DipA-B5-Asp-3COOHF-dAla-Ala-Phe-Lys*3-PyrS2-3Thi-BztA-GlnR*3-Ala-NH2 |
| I-1004 | Ac-PL3-Asp-DipA-B5-Asp-3COOHF-Aib-Ser-Phe-Lys*3-PyrS2-3Thi-BztA-GlnR*3-Ala-NH2 |
| I-1005 | Ac-PL3-Asp-DipA-B5-Asp-3COOHF-Aib-Thr-Phe-Lys*3-PyrS2-3Thi-BztA-GlnR*3-Ala-NH2 |
| I-1006 | Ac-PL3-Asp-DipA-B5-Asp-3COOHF-Aib-aThr-Phe-Lys*3-PyrS2-3Thi-BztA-GlnR*3-Ala-NH2 |
| I-1007 | Ac-PL3-Asp-DipA-B5-Asp-3COOHF-Aib-aThr-Phe-Lys*3-PyrS2-3Thi-BztA-GlnR*3-Ala-NH2 |
| I-1008 | Ac-PL3-Asp-DipA-B5-Asp-3COOHF-Aib-Gly-Phe-Lys*3-PyrS2-3Thi-BztA-GlnR*3-Ala-NH2 |
| I-1009 | Ac-PL3-Asp-DipA-B5-Asp-3COOHF-Aib-Gly-Phe-Lys*3-PyrS2-3Thi-BztA-GlnR*3-Ala-NH2 |
| I-1010 | Ac-PL3-Asp-Ile-B5-Asp-3COOHF-Aib-Ala-Phe-Lys*3-PyrS2-3Thi-34ClF-GlnR*3-Ala-NH2 |
| I-1011 | Ac-PL3-Asp-Npg-B5-Asp-3COOHF-CyLeu-Ser-Phe-Lys*3-PyrS2-3Thi-BztA-GlnR*3-Ala-NH2 |
| I-1012 | Ac-PL3-Asp-Npg-B5-Asp-3COOHF-Phe-Ser-Phe-Lys*3-PyrS2-3Thi-BztA-GlnR*3-Ala-NH2 |
| I-1013 | Ac-PL3-Asp-Npg-B5-Asp-3COOHF-Val-Ser-Phe-Lys*3-PyrS2-3Thi-BztA-GlnR*3-Ala-NH2 |
| I-1014 | Ac-PL3-Asp-Npg-B5-Asp-3COOHF-CyLeu-Gly-Phe-Lys*3-PyrS2-3Thi-BztA-GlnR*3-Ala-NH2 |
| I-1015 | Ac-PL3-Asp-Npg-B5-Asp-3COOHF-Phe-Gly-Phe-Lys*3-PyrS2-3Thi-BztA-GlnR*3-Ala-NH2 |
| I-1016 | Ac-PL3-Asp-Npg-B5-Asp-3COOHF-Val-Gly-Phe-Lys*3-PyrS2-3Thi-BztA-GlnR*3-Ala-NH2 |
| I-1017 | Ac-PL3-Asp-Npg-B5-Asp-3COOHF-CyLeu-nLeu-Phe-Lys*3-PyrS2-3Thi-BztA-GlnR*3-Ala-NH2 |
| I-1018 | Ac-PL3-Asp-Npg-B5-Asp-3COOHF-Phe-nLeu-Phe-Lys*3-PyrS2-3Thi-BztA-GlnR*3-Ala-NH2 |
| I-1019 | Ac-PL3-Asp-Npg-B5-Asp-3COOHF-Val-nLeu-Phe-Lys*3-PyrS2-3Thi-BztA-GlnR*3-Ala-NH2 |
| I-1020 | Ac-PL3-Asp-Npg-B5-Asp-3COOHF-CyLeu-aThr-Phe-Lys*3-PyrS2-3Thi-BztA-GlnR*3-Ala-NH2 |
| I-1021 | Ac-PL3-Asp-Npg-B5-Asp-3COOHF-Val-aThr-Phe-Lys*3-PyrS2-3Thi-BztA-GlnR*3-Ala-NH2 |
| I-1022 | 5hexenyl-MePro-Asp-S3-R5-Asp-3COOHF-Aib-Ala-Phe-Lys*3-PyrS2-3Thi-BztA-GlnR*3-Ala- |
| | NH2 |
| I-1023 | Ac-PL3-Asp-Npg-B5-Asp-3COOHF-Aib-Ala-Phe-Lys*3-PyrS1-3Thi-BztA-GlnR*3-Ala-NH2 |
| I-1024 | Ac-PL3-Glu-Npg-B5-Glu-3COOHF-Aib-Ala-Phe-Lys*3-PyrS2-3Thi-34ClF-GlnR*3-NH2 |
| I-1025 | 2PyBu-PL3-Glu-Npg-B5-Glu-3COOHF-Aib-Ala-Phe-Lys*3-PyrS2-3Thi-BztA-GlnR*3-Ala-NH2 |
| I-1026 | Ac-PL3-Asp-Npg-B5-Asp-3COOHF-[Ac]Acp-Ala-Phe-Lys*3-PyrS2-3Thi-BztA-GlnR*3-Ala-NH2 |
| I-1027 | Ac-PL3-Asp-Npg-B5-Asp-3COOHF-[Ac]Acp-Ala-Phe-Lys*3-PyrS2-3Thi-BztA-GlnR*3-Ala-NH2 |
| I-1028 | Ac-PL3-Asp-Npg-B5-Asp-3COOHF-[Phc]Acp-Ala-Phe-Lys*3-PyrS2-3Thi-BztA-GlnR*3-Ala-NH2 |
| I-1029 | Ac-PL3-Asp-Npg-B5-Asp-3COOHF-[Phc]Acp-Ala-Phe-Lys*3-PyrS2-3Thi-BztA-GlnR*3-Ala-NH2 |
| I-1030 | Ac-PL3-Asp-Npg-B5-Asp-3COOHF-[isovaleryl]Acp-Ala-Phe-Lys*3-PyrS2-3Thi-BztA-GlnR*3- |
| | Ala-NH2 |
| I-1031 | Ac-PL3-Asp-Npg-B5-Asp-3COOHF-[Ac]PyrSa-Ala-Phe-Lys*3-PyrS2-3Thi-BztA-GlnR*3-Ala- |
| | NH2 |
| I-1032 | Ac-PL3-Asp-Npg-B5-Asp-3COOHF-[Phc]PyrSa-Ala-Phe-Lys*3-PyrS2-3Thi-BztA-GlnR*3-Ala- |
| | NH2 |
| I-1033 | Ac-PL3-Asp-Npg-B5-Asp-3COOHF-[isovaleryl]PyrSa-Ala-Phe-Lys*3-PyrS2-3Thi-BztA-GlnR*3- |
| | Ala-NH2 |
| I-1034 | Ac-PL3-Asp-Npg-B5-Asp-3COOHF-[Ac]PyrRa-Ala-Phe-Lys*3-PyrS2-3Thi-BztA-GlnR*3-Ala- |
| | NH2 |
| I-1035 | Ac-PL3-Asp-Npg-B5-Asp-3COOHF-[Phc]PyrRa-Ala-Phe-Lys*3-PyrS2-3Thi-BztA-GlnR*3-Ala- |
| | NH2 |
| I-1036 | Ac-PL3-Asp-Npg-B5-Asp-3COOHF-[isovaleryl]PyrRa-Ala-Phe-Lys*3-PyrS2-3Thi-BztA-GlnR*3- |
| | Ala-NH2 |
| I-1037 | Ac-PL3-Asp-Chg-B5-Asp-3COOHF-Aib-Ala-Phe-Lys*3-PyrS2-3Thi-34ClF-GlnR*3-NH2 |
| I-1038 | Ac-PL3-Asp-Cha-B5-Asp-3COOHF-Aib-Ala-Phe-Lys*3-PyrS2-3Thi-34ClF-GlnR*3-NH2 |
| I-1039 | Ac-PL3-Asp-Cha-B5-Asp-3COOHF-Aib-Ala-Phe-Lys*3-PyrS2-3Thi-34ClF-GlnR*3-NH2 |
| I-1040 | Ac-PL3-Asp-Ile-B5-Asp-3COOHF-CyLeu-Ala-Phe-Lys*3-PyrS2-3Thi-34ClF-GlnR*3-NH2 |
| I-1041 | Ac-PL3-Asp-Ile-B5-Asp-3COOHF-CyLeu-Ala-Phe-Lys*3-PyrS2-3Thi-34ClF-GlnR*3-NH2 |
| I-1042 | Ac-PL3-Asp-Npg-B5-Asp-3COOHF-CyLeu-Ala-Phe-Lys*3-PyrS2-3Thi-34ClF-GlnR*3-NH2 |
| I-1043 | Ac-PL3-Asp-Npg-B5-Asp-3COOHF-CyLeu-Ala-Phe-Lys*3-PyrS2-3Thi-34ClF-GlnR*3-NH2 |
| I-1044 | Ac-PL3-Asp-Chg-B5-Asp-3COOHF-CyLeu-Ala-Phe-Lys*3-PyrS2-3Thi-34ClF-GlnR*3-NH2 |
| I-1045 | Ac-PL3-Asp-Chg-B5-Asp-3COOHF-CyLeu-Ala-Phe-Lys*3-PyrS2-3Thi-34ClF-GlnR*3-NH2 |
| I-1046 | Ac-PL3-Asp-Cha-B5-Asp-3COOHF-CyLeu-Ala-Phe-Lys*3-PyrS2-3Thi-34ClF-GlnR*3-NH2 |
| I-1047 | Ac-PL3-Asp-Cha-B5-Asp-3COOHF-CyLeu-Ala-Phe-Lys*3-PyrS2-3Thi-34ClF-GlnR*3-NH2 |
| I-1048 | Ac-PL3-Asp-Chg-B5-Asp-3COOHF-Aib-Ala-Phe-Lys*3-PyrS2-Phe-34ClF-GlnR*3-NH2 |
| I-1049 | Ac-PL3-Asp-Chg-B5-Asp-3COOHF-Aib-Ala-Phe-Lys*3-PyrS2-Phe-34ClF-GlnR*3-NH2 |
| I-1050 | Ac-PL3-Asp-Cha-B5-Asp-3COOHF-Aib-Ala-Phe-Lys*3-PyrS2-Phe-34ClF-GlnR*3-NH2 |
| I-1051 | Ac-PL3-Asp-Cha-B5-Asp-3COOHF-Aib-Ala-Phe-Lys*3-PyrS2-Phe-34ClF-GlnR*3-NH2 |
| I-1052 | Ac-PL3-Asp-Ile-B5-Asp-3COOHF-CyLeu-Ala-Phe-Lys*3-PyrS2-Phe-34ClF-GlnR*3-NH2 |
| I-1053 | Ac-PL3-Asp-Ile-B5-Asp-3COOHF-CyLeu-Ala-Phe-Lys*3-PyrS2-Phe-34ClF-GlnR*3-NH2 |
| I-1054 | Ac-PL3-Asp-Npg-B5-Asp-3COOHF-CyLeu-Ala-Phe-Lys*3-PyrS2-Phe-34ClF-GlnR*3-NH2 |
| I-1055 | Ac-PL3-Asp-Npg-B5-Asp-3COOHF-CyLeu-Ala-Phe-Lys*3-PyrS2-Phe-34ClF-GlnR*3-NH2 |
| I-1056 | Ac-PL3-Asp-Chg-B5-Asp-3COOHF-CyLeu-Ala-Phe-Lys*3-PyrS2-Phe-34ClF-GlnR*3-NH2 |
| I-1057 | Ac-PL3-Asp-Chg-B5-Asp-3COOHF-CyLeu-Ala-Phe-Lys*3-PyrS2-Phe-34ClF-GlnR*3-NH2 |
| I-1058 | Ac-PL3-Asp-Cha-B5-Asp-3COOHF-CyLeu-Ala-Phe-Lys*3-PyrS2-Phe-34ClF-GlnR*3-NH2 |
| I-1059 | Ac-PL3-Asp-Cha-B5-Asp-3COOHF-CyLeu-Ala-Phe-Lys*3-PyrS2-Phe-34ClF-GlnR*3-NH2 |
| I-1060 | Ac-PL3-Asp-Npg-B5-Asp-SbMeAsp-Aib-Ala-Phe-Lys*3-PyrS2-Phe-34ClF-GlnR*3-NH2 |
| I-1061 | Ac-PL3-Asp-Npg-B5-Asp-bMe2Asp-Aib-Ala-Phe-Lys*3-PyrS2-Phe-34ClF-GlnR*3-NH2 |
| I-1062 | 1Imidac-PL3-Asp-Npg-B5-Asp-3COOHF-Aib-Ala-Phe-Lys*3-PyrS2-3Thi-BztA-GlnR*3-Ala-NH2 |
| I-1063 | 2F2PyAc-PL3-Asp-Npg-B5-Asp-3COOHF-Aib-Ala-Phe-Lys*3-PyrS2-3Thi-BztA-GlnR*3-Ala- |
| | NH2 |
| I-1064 | 2IAPAc-PL3-Asp-Npg-B5-Asp-3COOHF-Aib-Ala-Phe-Lys*3-PyrS2-3Thi-BztA-GlnR*3-Ala-NH2 |
| I-1065 | 124TriPr-PL3-Asp-Npg-B5-Asp-3COOHF-Aib-Ala-Phe-Lys*3-PyrS2-3Thi-BztA-GlnR*3-Ala- |
| | NH2 |
| I-1066 | 6QuiAc-PL3-Asp-Npg-B5-Asp-3COOHF-Aib-Ala-Phe-Lys*3-PyrS2-3Thi-BztA-GlnR*3-Ala-NH2 |
| I-1067 | 3Py Ac-PL3-Asp-Npg-B5-Asp-3COOHF-Aib-Ala-Phe-Lys*3-PyrS2-3Thi-BztA-GlnR*3-Ala-NH2 |
| I-1068 | 123TriAc-PL3-Asp-Npg-B5-Asp-3COOHF-Aib-Ala-Phe-Lys*3-PyrS2-3Thi-BztA-GlnR*3-Ala- |
| | NH2 |
| I-1069 | 1Pyrazole Ac-PL3-Asp-Npg-B5-Asp-3COOHF-Aib-Ala-Phe-Lys*3-PyrS2-3Thi-BztA-GlnR*3-Ala- |
| | NH2 |
| I-1070 | 4PyPrpc-PL3-Asp-Npg-B5-Asp-3COOHF-Aib-Ala-Phe-Lys*3-PyrS2-3Thi-BztA-GlnR*3-Ala-NH2 |
| I-1071 | 4PyPrpc-PL3-Asp-Npg-B5-Asp-3COOHF-Aib-Ala-Phe-Lys*3-PyrS2-3Thi-BztA-GlnR*3-Ala-NH2 |
| I-1072 | 3PyPrpc-PL3-Asp-Npg-B5-Asp-3COOHF-Aib-Ala-Phe-Lys*3-PyrS2-3Thi-BztA-GlnR*3-Ala-NH2 |
| I-1073 | 5PymAc-PL3-Asp-Npg-B5-Asp-3COOHF-Aib-Ala-Phe-Lys*3-PyrS2-3Thi-BztA-GlnR*3-Ala-NH2 |
| I-1074 | 1PydoneAc-PL3-Asp-Npg-B5-Asp-3COOHF-Aib-Ala-Phe-Lys*3-PyrS2-3Thi-BztA-GlnR*3-Ala- |
| | NH2 |
| I-1075 | 124TriAc-PL3-Asp-Npg-B5-Asp-3COOHF-Aib-Ala-Phe-Lys*3-PyrS2-3Thi-BztA-GlnR*3-Ala- |
| | NH2 |
| I-1076 | 3IAPAc-PL3-Asp-Npg-B5-Asp-3COOHF-Aib-Ala-Phe-Lys*3-PyrS2-3Thi-BztA-GlnR*3-Ala-NH2 |
| I-1077 | 3IAPAc-PL3-Asp-Npg-B5-Asp-3COOHF-Aib-Ala-Phe-Lys*3-PyrS2-3Thi-BztA-GlnR*3-Ala-NH2 |
| I-1078 | Me2NAc-PL3-Asp-Npg-B5-Asp-3COOHF-Aib-Ala-Phe-Lys*3-PyrS2-3Thi-BztA-GlnR*3-Ala- |
| | NH2 |
| I-1079 | 4MePipzPrpC-PL3-Asp-Npg-B5-Asp-3COOHF-Aib-Ala-Phe-Lys*3-PyrS2-3Thi-BztA-GlnR*3- |
| | Ala-NH2 |
| I-1080 | 4MePipzPrpC-PL3-Asp-Npg-B5-Asp-3COOHF-Aib-Ala-Phe-Lys*3-PyrS2-3Thi-BztA-GlnR*3- |
| | Ala-NH2 |
| I-1081 | MePipAc-PL3-Asp-Npg-B5-Asp-3COOHF-Aib-Ala-Phe-Lys*3-PyrS2-3Thi-BztA-GlnR*3-Ala- |
| | NH2 |
| I-1082 | MePipAc-PL3-Asp-Npg-B5-Asp-3COOHF-Aib-Ala-Phe-Lys*3-PyrS2-3Thi-BztA-GlnR*3-Ala- |
| | NH2 |
| I-1083 | MeImid4SO2-PL3-Asp-Npg-B5-Asp-3COOHF-Aib-Ala-Phe-Lys*3-PyrS2-3Thi-BztA-GlnR*3- |
| | Ala-NH2 |
| I-1084 | MeImid4SO2-PL3-Asp-Npg-B5-Asp-3COOHF-Aib-Ala-Phe-Lys*3-PyrS2-3Thi-BztA-GlnR*3- |
| | Ala-NH2 |
| I-1085 | 8QuiSO2-PL3-Asp-Npg-B5-Asp-3COOHF-Aib-Ala-Phe-Lys*3-PyrS2-3Thi-BztA-GlnR*3-Ala- |
| | NH2 |
| I-1086 | Ac-PL3-Asp-Npg-B5-Asp-3COOHF-Aib-Ala-Phe-TriAzLys*3-PyrS2-3Thi-34ClF-sAla*3-NH2 |
| I-1087 | Ac-PL3-Asp-Npg-B5-Asp-3COOHF-Aib-Ala-Phe-TriAzLys*3-PyrS2-3Thi-34ClF-sAla*3-NH2 |
| I-1088 | Ac-PL3-Asp-Npg-B5-Asp-3COOHF-Aib-Ala-Phe-TriAzLys*3-PyrS2-3Thi-34ClF-sAla*3-Ala- |
| | NH2 |
| I-1089 | Ac-PL3-Asp-Ile-B5-Asp-3COOHF-Aib-Ala-Phe-TriAzLys*3-PyrS2-3Thi-34ClF-sAla*3-NH2 |
| I-1090 | Ac-PL3-Asp-Ile-B5-Asp-3COOHF-Phe-Ala-Phe-TriAzLys*3-PyrS2-3Thi-34ClF-sAla*3-NH2 |
| I-1091 | Ac-PL3-Asp-Ile-B5-Asp-3COOHF-Phe-Ala-Phe-TriAzLys*3-PyrS2-3Thi-34ClF-sAla*3-Ala-NH2 |
| I-1092 | Ac-PL3-Asp-Npg-B5-Asp-3COOHF-Aib-Ala-Phe-TriAzLys*3-PyrS2-Phe-34ClF-sAla*3-NH2 |
| I-1093 | Ac-PL3-Asp-Npg-B5-Asp-3COOHF-Aib-Ala-Phe-TriAzLys*3-PyrS2-Phe-34ClF-sAla*3-Ala-NH2 |
| I-1094 | Ac-PL3-Asp-Ile-B5-Asp-3COOHF-Aib-Ala-Phe-TriAzLys*3-PyrS2-Phe-34ClF-sAla*3-NH2 |
| I-1095 | Ac-PL3-Asp-Ile-B5-Asp-3COOHF-Aib-Ala-Phe-TriAzLys*3-PyrS2-Phe-34ClF-sAla*3-Ala-NH2 |
| I-1096 | Ac-PL3-Asp-Ile-B5-Asp-3COOHF-Phe-Ala-Phe-TriAzLys*3-PyrS2-Phe-34ClF-sAla*3-NH2 |
| I-1097 | Ac-PL3-Asp-Ile-B5-Asp-3COOHF-Phe-Ala-Phe-TriAzLys*3-PyrS2-Phe-34ClF-sAla*3-Ala-NH2 |
| I-1098 | Ac-PL3-Asp-Npg-B5-Asp-3COOHF-Aib-Ala-Phe-Lys*3-PyrS2-3Thi-34MeF-GlnR*3-NH2 |
| I-1099 | Ac-PL3-Asp-Ile-B5-Asp-3COOHF-Aib-Ala-Phe-Lys*3-PyrS2-3Thi-34MeF-GlnR*3-Ala-NH2 |
| I-1100 | Ac-PL3-Asp-Ile-B5-Asp-3COOHF-Aib-Ala-Phe-Lys*3-PyrS2-3Thi-34MeF-GlnR*3-NH2 |
| I-1101 | Ac-PL3-Asp-Ile-B5-Asp-3COOHF-Phe-Ala-Phe-Lys*3-PyrS2-3Thi-34MeF-GlnR*3-Ala-NH2 |
| I-1102 | Ac-PL3-Asp-Ile-B5-Asp-3COOHF-Phe-Ala-Phe-Lys*3-PyrS2-3Thi-34MeF-GlnR*3-NH2 |
| I-1103 | Ac-PL3-Asp-Npg-B5-Asp-3COOHF-Aib-Ala-Phe-Lys*3-PyrS2-Phe-34MeF-GlnR*3-NH2 |
| I-1104 | Ac-PL3-Asp-Ile-B5-Asp-3COOHF-Aib-Ala-Phe-Lys*3-PyrS2-Phe-34MeF-GlnR*3-Ala-NH2 |
| I-1105 | Ac-PL3-Asp-Ile-B5-Asp-3COOHF-Aib-Ala-Phe-Lys*3-PyrS2-Phe-34MeF-GlnR*3-NH2 |
| I-1106 | Ac-PL3-Asp-Ile-B5-Asp-3COOHF-Phe-Ala-Phe-Lys*3-PyrS2-Phe-34MeF-GlnR*3-Ala-NH2 |
| I-1107 | Ac-PL3-Asp-Ile-B5-Asp-3COOHF-Phe-Ala-Phe-Lys*3-PyrS2-Phe-34MeF-GlnR*3-NH2 |
| I-1108 | Ac-PL3-Asp-DipA-B5-Asp-3COOHF-Aib-Ala-Phe-Lys*3-PyrS2-3Thi-34ClF-GlnR*3-NH2 |
| I-1109 | Ac-PL3-Asp-DipA-B5-Asp-3COOHF-Aib-Ala-Phe-Lys*3-PyrS2-3Thi-34ClF-GlnR*3-Ala-NH2 |
| I-1110 | Ac-PL3-Asp-DipA-B5-Asp-3COOHF-Aib-Ala-Phe-Lys*3-PyrS2-3Thi-34ClF-GlnR*3-Ser-NH2 |
| I-1111 | Ac-PL3-Asp-DipA-B5-Asp-3COOHF-Ala-Ala-Phe-Lys*3-PyrS2-3Thi-34ClF-GlnR*3-NH2 |
| I-1112 | Ac-PL3-Asp-DipA-B5-Asp-3COOHF-Ala-Ala-Phe-Lys*3-PyrS2-3Thi-34ClF-GlnR*3-Ala-NH2 |
| I-1113 | Ac-PL3-Asp-DipA-B5-Asp-3COOHF-Ala-Ala-Phe-Lys*3-PyrS2-3Thi-34ClF-GlnR*3-Ser-NH2 |
| I-1114 | Ac-PL3-Asp-DipA-B5-Asp-3COOHF-Phe-Ala-Phe-Lys*3-PyrS2-3Thi-34ClF-GlnR*3-NH2 |
| I-1115 | Ac-PL3-Asp-DipA-B5-Asp-3COOHF-Phe-Ala-Phe-Lys*3-PyrS2-3Thi-34ClF-GlnR*3-Ala-NH2 |
| I-1116 | Ac-PL3-Asp-DipA-B5-Asp-3COOHF-Phe-Ala-Phe-Lys*3-PyrS2-3Thi-34ClF-GlnR*3-Ser-NH2 |
| I-1117 | Ac-PL3-Asp-DipA-B5-Asp-3COOHF-CyLeu-Ala-Phe-Lys*3-PyrS2-3Thi-34ClF-GlnR*3-NH2 |
| I-1118 | Ac-PL3-Asp-DipA-B5-Asp-3COOHF-CyLeu-Ala-Phe-Lys*3-PyrS2-3Thi-34ClF-GlnR*3-Ala-NH2 |
| I-1119 | Ac-PL3-Asp-DipA-B5-Asp-3COOHF-CyLeu-Ala-Phe-Lys*3-PyrS2-3Thi-34ClF-GlnR*3-Ser-NH2 |
| I-1120 | Ac-PL3-Asp-Npg-B5-Asp-3COOHF-Aib-Ala-Phe-TriAzLys*3-PyrSc72SMe3ROMe-3Thi-BztA- |
| | sAla*3-Ala-NH2 |
| I-1121 | Ac-PL3-Asp-Npg-B5-Asp-3COOHF-Aib-Ala-Phe-TriAzLys*3-PyrSc72RMe3SOMe-3Thi-BztA- |
| | sAla*3-Ala-NH2 |
| I-1122 | Ac-PL3-Asp-Npg-B5-Asp-3COOHF-Aib-Ala-Phe-TriAzLys*3-[SMeIso2]PyrSc704-3Thi-BztA- |
| | sAla*3-Ala-NH2 |
| I-1123 | Ac-PL3-Asp-Npg-B5-Asp-3COOHF-Aib-Ala-Phe-TriAzLys*3-[RMeIso2]PyrSc704-3Thi-BztA- |
| | sAla*3-Ala-NH2 |
| I-1124 | Ac-PL3-Asp-Npg-B5-Asp-3COOHF-Aib-Ala-Phe-TriAzLys*3-PyrSc73Me2-3Thi-BztA-sAla*3- |
| | Ala-NH2 |
| I-1125 | Ac-PL3-Asp-Npg-B5-Asp-3COOHF-Aib-Ala-Phe-TriAzLys*3-PyrSc7-3Thi-BztA-sAla*3-Ala- |
| | NH2 |
| I-1126 | Ac-PL3-Asp-Npg-B5-Asp-3COOHF-Aib-Ala-Phe-TriAzLys*3-[CO][Ala]PyrSa-3Thi-BztA- |
| | sAla*3-Ala-NH2 |
| I-1127 | Ac-PL3-Asp-Npg-B5-Asp-3COOHF-Aib-Ala-Phe-TriAzLys*3-[CO][dAla]PyrSa-3Thi-BztA- |
| | sAla*3-Ala-NH2 |
| I-1128 | Ac-S5-Ser-Asp-Npg-B5-Asp-3COOHF-Aib-Ala-Phe-Lys*3-PyrS2-3Thi-BztA-GlnR*3-Ala-NH2 |
| I-1129 | Ac-S5-Ser-Asp-Npg-B5-Asp-3COOHF-Aib-Ala-Phe-Lys*3-PyrS2-3Thi-BztA-GlnR*3-Ala-NH2 |
| I-1130 | Ac-S5-Val-Asp-Npg-B5-Asp-3COOHF-Aib-Ala-Phe-Lys*3-PyrS2-3Thi-BztA-GlnR*3-Ala-NH2 |
| I-1131 | Ac-S5-Val-Asp-Npg-B5-Asp-3COOHF-Aib-Ala-Phe-Lys*3-PyrS2-3Thi-BztA-GlnR*3-Ala-NH2 |
| I-1132 | Ac-S5-Leu-Asp-Npg-B5-Asp-3COOHF-Aib-Ala-Phe-Lys*3-PyrS2-3Thi-BztA-GlnR*3-Ala-NH2 |
| I-1133 | Ac-S5-Leu-Asp-Npg-B5-Asp-3COOHF-Aib-Ala-Phe-Lys*3-PyrS2-3Thi-BztA-GlnR*3-Ala-NH2 |
| I-1134 | Ac-S5-Thr-Asp-Npg-B5-Asp-3COOHF-Aib-Ala-Phe-Lys*3-PyrS2-3Thi-BztA-GlnR*3-Ala-NH2 |
| I-1135 | Ac-S5-Thr-Asp-Npg-B5-Asp-3COOHF-Aib-Ala-Phe-Lys*3-PyrS2-3Thi-BztA-GlnR*3-Ala-NH2 |
| I-1136 | Ac-S5-Phe-Asp-Npg-B5-Asp-3COOHF-Aib-Ala-Phe-Lys*3-PyrS2-3Thi-BztA-GlnR*3-Ala-NH2 |
| I-1137 | Ac-S5-Phe-Asp-Npg-B5-Asp-3COOHF-Aib-Ala-Phe-Lys*3-PyrS2-3Thi-BztA-GlnR*3-Ala-NH2 |
| I-1138 | Ac-Pro-S5-Ala-Asp-Npg-B5-Asp-3COOHF-Aib-Ala-Phe-Lys*3-PyrS2-3Thi-BztA-GlnR*3-Ala- |
| | NH2 |
| I-1139 | Ac-Pro-S5-Ala-Asp-Npg-B5-Asp-3COOHF-Aib-Ala-Phe-Lys*3-PyrS2-3Thi-BztA-GlnR*3-Ala- |
| | NH2 |
| I-1140 | Ac-Ser-S5-Ala-Asp-Npg-B5-Asp-3COOHF-Aib-Ala-Phe-Lys*3-PyrS2-3Thi-BztA-GlnR*3-Ala- |
| | NH2 |
| I-1141 | Ac-Ser-S5-Ala-Asp-Npg-B5-Asp-3COOHF-Aib-Ala-Phe-Lys*3-PyrS2-3Thi-BztA-GlnR*3-Ala- |
| | NH2 |
| I-1142 | Ac-Ala-S5-Ala-Asp-Npg-B5-Asp-3COOHF-Aib-Ala-Phe-Lys*3-PyrS2-3Thi-BztA-GlnR*3-Ala- |
| | NH2 |
| I-1143 | Ac-Ala-S5-Ala-Asp-Npg-B5-Asp-3COOHF-Aib-Ala-Phe-Lys*3-PyrS2-3Thi-BztA-GlnR*3-Ala- |
| | NH2 |
| I-1144 | Ac-S5-Ser-Asp-Npg-B5-Asp-3COOHF-Aib-Ala-Phe-Lys*3-PyrS2-3Thi-BztA-GlnR*3-Ala-NH2 |
| I-1145 | Ac-S5-Val-Asp-Npg-B5-Asp-3COOHF-Aib-Ala-Phe-Lys*3-PyrS2-3Thi-BztA-GlnR*3-Ala-NH2 |
| I-1146 | Ac-S5-Leu-Asp-Npg-B5-Asp-3COOHF-Aib-Ala-Phe-Lys*3-PyrS2-3Thi-BztA-GlnR*3-Ala-NH2 |
| I-1147 | Ac-S5-Thr-Asp-Npg-B5-Asp-3COOHF-Aib-Ala-Phe-Lys*3-PyrS2-3Thi-BztA-GlnR*3-Ala-NH2 |
| I-1148 | Ac-S5-Phe-Asp-Npg-B5-Asp-3COOHF-Aib-Ala-Phe-Lys*3-PyrS2-3Thi-BztA-GlnR*3-Ala-NH2 |
| I-1149 | Ac-Pro-S5-Ala-Asp-Npg-B5-Asp-3COOHF-Aib-Ala-Phe-Lys*3-PyrS2-3Thi-BztA-GlnR*3-Ala- |
| | NH2 |
| I-1150 | Ac-Ser-S5-Ala-Asp-Npg-B5-Asp-3COOHF-Aib-Ala-Phe-Lys*3-PyrS2-3Thi-BztA-GlnR*3-Ala- |
| | NH2 |
| I-1151 | Ac-Ala-S5-Ala-Asp-Npg-B5-Asp-3COOHF-Aib-Ala-Phe-Lys*3-PyrS2-3Thi-BztA-GlnR*3-Ala- |
| | NH2 |
| I-1152 | Ac-PL3-Val-Npg-B5-Asp-3COOHF-Aib-Ala-Phe-Lys*3-PyrS2-3Thi-BztA-GlnR*3-Ala-NH2 |
| I-1153 | Ac-PL3-Npg-Npg-B5-Asp-3COOHF-Aib-Ala-Phe-Lys*3-PyrS2-3Thi-BztA-GlnR*3-Ala-NH2 |
| I-1154 | Ac-PL3-BztA-Npg-B5-Asp-3COOHF-Aib-Ala-Phe-Lys*3-PyrS2-3Thi-BztA-GlnR*3-Ala-NH2 |
| I-1155 | Ac-PL3-Asp-Npg-B5-Asp-3COOHF-Aib-Ala-Phe-Lys*3-PyrS2-3Thi-BztA-GlnR*3-Ala-Ala-Arg- |
| | Ala-Ala-Ala-Ala-NH2 |
| I-1156 | Ac-PL3-Asp-Npg-B5-Asp-3COOHF-Aib-Ala-Phe-Lys*3-PyrS2-3Thi-BztA-GlnR*3-Ala-Arg-Ala- |
| | Ala-Ala-Arg-Ala-NH2 |
| I-1157 | Ac-PL3-Asp-Npg-B5-Asp-3COOHF-Aib-Ala-Phe-Lys*3-PyrS2-3Thi-BztA-GlnR*3-Ala-Ala-Arg- |
| | Ala-Ala-Ala-Arg-NH2 |
| I-1158 | Ac-PL3-Asp-Npg-B5-Asp-3COOHF-Aib-Ala-Phe-Lys*3-PyrS2-3Thi-BztA-GlnR*3-Ala-Ala-Lys- |
| | Ala-Ala-Ala-Lys-NH2 |
| I-1159 | Ac-PL3-Asp-Npg-B5-Asp-3COOHF-Aib-Ala-Phe-Lys*3-PyrS2-3Thi-BztA-GlnR*3-Ala-Ala- |
| | GlnR**3-Ala-Ala-Ala-Lys**3-NH2 |
| I-1160 | Ac-PL3-Asp-Npg-B5-Asp-3COOHF-Aib-Ala-Phe-Lys*3-PyrS2-3Thi-BztA-GlnR*3-Ala-Ala-Ala- |
| | GlnR**3-Ala-Ala-Ala-Lys**3-NH2 |
| I-1161 | Ac-PL3-Asp-Npg-B5-Asp-3COOHF-Aib-Ala-Phe-Lys*3-PyrS2-c6Phe-BztA-GlnR*3-Ala-NH2 |
| I-1162 | Ac-PL3-Asn-Npg-B5-Asp-3COOHF-Aib-Ala-Phe-Lys*3-PyrS2-3Thi-BztA-GlnR*3-NH2 |
| I-1163 | Ac-PL3-Asn-DipA-B5-Asp-3COOHF-Aib-Ala-Phe-Lys*3-PyrS2-3Thi-BztA-GlnR*3-NH2 |
| I-1164 | Ac-PL3-Asn-DipA-B5-Asp-3COOHF-Aib-Ala-Phe-Lys*3-PyrS2-3Thi-BztA-GlnR*3-Ala-NH2 |
| I-1165 | Ac-PL3-Asn-Ile-B5-Asp-3COOHF-Aib-Ala-Phe-Lys*3-PyrS2-3Thi-BztA-GlnR*3-NH2 |
| I-1166 | Ac-PL3-Asn-Ile-B5-Asp-3COOHF-Aib-Ala-Phe-Lys*3-PyrS2-3Thi-BztA-GlnR*3-NH2 |
| I-1167 | Ac-PL3-Asn-Ile-B5-Asp-3COOHF-Aib-Ala-Phe-Lys*3-PyrS2-3Thi-BztA-GlnR*3-Ala-NH2 |
| I-1168 | Ac-PL3-Asn-Ile-B5-Asp-3COOHF-Aib-Ala-Phe-Lys*3-PyrS2-3Thi-BztA-GlnR*3-Ala-NH2 |
| I-1169 | Ac-PL3-Asn-Ile-B5-Asp-3COOHF-Aib-Ala-Phe-Lys*3-PyrS2-3Thi-BztA-GlnR*3-Ser-NH2 |
| I-1170 | Ac-PL3-Asn-Ile-B5-Asp-3COOHF-Aib-Ala-Phe-Lys*3-PyrS2-3Thi-BztA-GlnR*3-Ser-NH2 |
| I-1171 | Ac-PL3-Asn-Npg-B5-Asp-3COOHF-Aib-Ala-Phe-Lys*3-PyrS2-Phe-34ClF-GlnR*3-NH2 |
| I-1172 | Ac-PL3-Asn-Npg-B5-Asp-3COOHF-Aib-Ala-Phe-Lys*3-PyrS2-Phe-34ClF-GlnR*3-NH2 |
| I-1173 | Ac-PL3-Asn-Npg-B5-Asp-3COOHF-Aib-Ala-Phe-Lys*3-PyrS2-Phe-34ClF-GlnR*3-Ala-NH2 |
| I-1174 | Ac-PL3-Asn-Npg-B5-Asp-3COOHF-Aib-Ala-Phe-Lys*3-PyrS2-Phe-34ClF-GlnR*3-Ala-NH2 |
| I-1175 | Ac-PL3-Asn-DipA-B5-Asp-3COOHF-Aib-Ala-Phe-Lys*3-PyrS2-Phe-34ClF-GlnR*3-NH2 |
| I-1176 | Ac-PL3-Asn-DipA-B5-Asp-3COOHF-Aib-Ala-Phe-Lys*3-PyrS2-Phe-34ClF-GlnR*3-NH2 |
| I-1177 | Ac-PL3-Asn-DipA-B5-Asp-3COOHF-Aib-Ala-Phe-Lys*3-PyrS2-Phe-34ClF-GlnR*3-Ala-NH2 |
| I-1178 | Ac-PL3-Asn-Ile-B5-Asp-3COOHF-Aib-Ala-Phe-Lys*3-PyrS2-Phe-34ClF-GlnR*3-NH2 |
| I-1179 | Ac-PL3-Asn-Ile-B5-Asp-3COOHF-Aib-Ala-Phe-Lys*3-PyrS2-Phe-34ClF-GlnR*3-NH2 |
| I-1180 | Ac-PL3-Asn-Ile-B5-Asp-3COOHF-Aib-Ala-Phe-Lys*3-PyrS2-Phe-34ClF-GlnR*3-Ala-NH2 |
| I-1181 | Ac-PL3-Asp-Npg-B5-Asp-3COOHF-Aib-Ala-Phe-Lys*3-PyrS2-3Thi-BztA-GlnR*3-[mPEG4]Lys- |
| | NH2 |
| I-1182 | Ac-PL3-Asp-Npg-B5-Asp-3COOHF-Aib-Ala-Phe-Lys*3-PyrS2-3Thi-BztA-GlnR*3-[mPEG8]Lys- |
| | NH2 |
| I-1183 | Ac-PL3-Asp-Npg-B5-Asp-3COOHF-Aib-Ala-Phe-Lys*3-PyrS2-3Thi-BztA-GlnR*3- |
| | [mPEG16]Lys-NH2 |
| I-1184 | mPEG4-PL3-Asp-Npg-B5-Asp-3COOHF-Aib-Ala-Phe-Lys*3-PyrS2-3Thi-BztA-GlnR*3-Ala-NH2 |
| I-1185 | mPEG8-PL3-Asp-Npg-B5-Asp-3COOHF-Aib-Ala-Phe-Lys*3-PyrS2-3Thi-BztA-GlnR*3-Ala-NH2 |
| I-1186 | mPEG16-PL3-Asp-Npg-B5-Asp-3COOHF-Aib-Ala-Phe-Lys*3-PyrS2-3Thi-BztA-GlnR*3-Ala- |
| | NH2 |
| I-1187 | mPEG24-PL3-Asp-Npg-B5-Asp-3COOHF-Aib-Ala-Phe-Lys*3-PyrS2-3Thi-BztA-GlnR*3-Ala- |
| | NH2 |
| I-1188 | Ac-PL3-Asp-Npg-B5-Asp-3BOH2F-Aib-Ala-Phe-Lys*3-PyrS2-3Thi-BztA-GlnR*3-Ala-NH2 |
| I-1189 | Ac-PL3-Asp-Npg-B5-Asp-4BOH2F-Aib-Ala-Phe-Lys*3-PyrS2-3Thi-BztA-GlnR*3-Ala-NH2 |
| I-1190 | Ac-PL3-Asp-Npg-B5-Asp-4BOH2F-Aib-Ala-Phe-Lys*3-PyrS2-3Thi-BztA-GlnR*3-Ala-NH2 |
| I-1191 | Ac-S5-Ala-Glu-Npg-B5-Asp-3COOHF-Aib-Ala-Phe-Lys*3-PyrS2-3Thi-BztA-GlnR*3-Ala-NH2 |
| I-1192 | Ac-S5-Ala-Glu-Npg-B5-Asp-3COOHF-Aib-Ala-Phe-Lys*3-PyrS2-3Thi-BztA-GlnR*3-Ala-NH2 |
| I-1193 | Ac-S5-Ala-Asn-Npg-B5-Asp-3COOHF-Aib-Ala-Phe-Lys*3-PyrS2-3Thi-BztA-GlnR*3-Ala-NH2 |
| I-1194 | Ac-S5-Ala-Asn-Npg-B5-Asp-3COOHF-Aib-Ala-Phe-Lys*3-PyrS2-3Thi-BztA-GlnR*3-Ala-NH2 |
| I-1195 | Ac-S5-Ala-Ser-Npg-B5-Asp-3COOHF-Aib-Ala-Phe-Lys*3-PyrS2-3Thi-BztA-GlnR*3-Ala-NH2 |
| I-1196 | Ac-S5-Ala-Ser-Npg-B5-Asp-3COOHF-Aib-Ala-Phe-Lys*3-PyrS2-3Thi-BztA-GlnR*3-Ala-NH2 |
| I-1197 | Ac-S5-Ala-Hse-Npg-B5-Asp-3COOHF-Aib-Ala-Phe-Lys*3-PyrS2-3Thi-BztA-GlnR*3-Ala-NH2 |
| I-1198 | Ac-S5-Ala-Hse-Npg-B5-Asp-3COOHF-Aib-Ala-Phe-Lys*3-PyrS2-3Thi-BztA-GlnR*3-Ala-NH2 |
| I-1199 | Ac-S5-Ala-Asp-Npg-B5-Glu-3COOHF-Aib-Ala-Phe-Lys*3-PyrS2-3Thi-BztA-GlnR*3-Ala-NH2 |
| I-1200 | Ac-S5-Ala-Asp-Npg-B5-Glu-3COOHF-Aib-Ala-Phe-Lys*3-PyrS2-3Thi-BztA-GlnR*3-Ala-NH2 |
| I-1201 | Ac-S5-Ala-Asp-Npg-B5-Asn-3COOHF-Aib-Ala-Phe-Lys*3-PyrS2-3Thi-BztA-GlnR*3-Ala-NH2 |
| I-1202 | Ac-S5-Ala-Asp-Npg-B5-Asn-3COOHF-Aib-Ala-Phe-Lys*3-PyrS2-3Thi-BztA-GlnR*3-Ala-NH2 |
| I-1203 | Ac-S5-Ala-Asp-Npg-B5-Ser-3COOHF-Aib-Ala-Phe-Lys*3-PyrS2-3Thi-BztA-GlnR*3-Ala-NH2 |
| I-1204 | Ac-S5-Ala-Asp-Npg-B5-Ser-3COOHF-Aib-Ala-Phe-Lys*3-PyrS2-3Thi-BztA-GlnR*3-Ala-NH2 |
| I-1205 | Ac-S5-Ala-Asp-Npg-B5-Hse-3COOHF-Aib-Ala-Phe-Lys*3-PyrS2-3Thi-BztA-GlnR*3-Ala-NH2 |
| I-1206 | Ac-S5-Ala-Asp-Npg-B5-Hse-3COOHF-Aib-Ala-Phe-Lys*3-PyrS2-3Thi-BztA-GlnR*3-Ala-NH2 |
| I-1207 | Ac-S5-Ala-Glu-Npg-B5-Glu-3COOHF-Aib-Ala-Phe-Lys*3-PyrS2-3Thi-BztA-GlnR*3-Ala-NH2 |
| I-1208 | Ac-S5-Ala-Glu-Npg-B5-Glu-3COOHF-Aib-Ala-Phe-Lys*3-PyrS2-3Thi-BztA-GlnR*3-Ala-NH2 |
| I-1209 | Ac-S5-Glu-Glu-Npg-B5-Asp-3COOHF-Aib-Ala-Phe-Lys*3-PyrS2-3Thi-BztA-GlnR*3-Ala-NH2 |
| I-1210 | Ac-S5-Glu-Glu-Npg-B5-Asp-3COOHF-Aib-Ala-Phe-Lys*3-PyrS2-3Thi-BztA-GlnR*3-Ala-NH2 |
| I-1211 | Ac-S5-Glu-Asp-Npg-B5-Glu-3COOHF-Aib-Ala-Phe-Lys*3-PyrS2-3Thi-BztA-GlnR*3-Ala-NH2 |
| I-1212 | Ac-S5-Glu-Asp-Npg-B5-Glu-3COOHF-Aib-Ala-Phe-Lys*3-PyrS2-3Thi-BztA-GlnR*3-Ala-NH2 |
| I-1213 | Ac-PL3-Asp-Npg-B5-Asp-3COOHF-Ala-Ala-Phe-Leu-[Red][Gly]Dap-3thi-BztA-Ala-GlnR-NH2 |
| I-1214 | Ac-PL3-Asp-Npg-B5-Asp-3COOHF-Ala-Ala-Phe-Leu-[Red][NHPent]Dap-3thi-BztA-Ala-GlnR- |
| | NH2 |
| I-1215 | Ac-PL3-Asp-Npg-B5-Asp-3COOHF-Ala-Ala-Phe-Leu-[SBut][CH2CH2NH]Dap-3thi-BztA-Ala- |
| | GlnR-NH2 |
| I-1216 | Ac-PL3-Asp-Npg-B5-Asp-3COOHF-Ala-Ala-Phe-Leu-[SBut][CH2CH2CH2NH]Dap-3thi-BztA- |
| | Ala-GlnR-NH2 |
| I-1217 | Ac-S5-Ala-Glu-Npg-B5-Asp-3COOHF-Aib-Ala-Phe-Lys*3-PyrS2-3Thi-BztA-GlnR*3-Ala-NH2 |
| I-1218 | Ac-S5-Ala-Asn-Npg-B5-Asp-3COOHF-Aib-Ala-Phe-Lys*3-PyrS2-3Thi-BztA-GlnR*3-Ala-NH2 |
| I-1219 | Ac-S5-Ala-Ser-Npg-B5-Asp-3COOHF-Aib-Ala-Phe-Lys*3-PyrS2-3Thi-BztA-GlnR*3-Ala-NH2 |
| I-1220 | Ac-S5-Ala-Hse-Npg-B5-Asp-3COOHF-Aib-Ala-Phe-Lys*3-PyrS2-3Thi-BztA-GlnR*3-Ala-NH2 |
| I-1221 | Ac-S5-Ala-Asp-Npg-B5-Glu-3COOHF-Aib-Ala-Phe-Lys*3-PyrS2-3Thi-BztA-GlnR*3-Ala-NH2 |
| I-1222 | Ac-S5-Ala-Asp-Npg-B5-Asn-3COOHF-Aib-Ala-Phe-Lys*3-PyrS2-3Thi-BztA-GlnR*3-Ala-NH2 |
| I-1223 | Ac-S5-Ala-Asp-Npg-B5-Ser-3COOHF-Aib-Ala-Phe-Lys*3-PyrS2-3Thi-BztA-GlnR*3-Ala-NH2 |
| I-1224 | Ac-S5-Ala-Asp-Npg-B5-Hse-3COOHF-Aib-Ala-Phe-Lys*3-PyrS2-3Thi-BztA-GlnR*3-Ala-NH2 |
| I-1225 | Ac-S5-Ala-Glu-Npg-B5-Glu-3COOHF-Aib-Ala-Phe-Lys*3-PyrS2-3Thi-BztA-GlnR*3-Ala-NH2 |
| I-1226 | Ac-S5-Glu-Glu-Npg-B5-Asp-3COOHF-Aib-Ala-Phe-Lys*3-PyrS2-3Thi-BztA-GlnR*3-Ala-NH2 |
| I-1227 | Ac-S5-Glu-Asp-Npg-B5-Glu-3COOHF-Aib-Ala-Phe-Lys*3-PyrS2-3Thi-BztA-GlnR*3-Ala-NH2 |
| I-1228 | Ac-S5-Glu-Glu-Npg-B5-Glu-3COOHF-Aib-Ala-Phe-Lys*3-PyrS2-3Thi-BztA-GlnR*3-Ala-NH2 |
| I-1229 | Ac-S5-Ala-Asp-Npg-B5-Asp-3COOHF-Aib-Ala-Phe-Lys*3-PyrS2-Phe-34ClF-GlnR*3-Ala-NH2 |
| I-1230 | Ac-S5-Ala-Asp-Npg-B5-Asp-3COOHF-Aib-Ala-Phe-Lys*3-PyrS2-Phe-34ClF-GlnR*3-NH2 |
| I-1231 | Ac-S5-Ala-Asp-Npg-B5-Asp-3COOHF-Aib-Ala-Phe-Lys*3-PyrS2-3Thi-34ClF-GlnR*3-Ala-NH2 |
| I-1232 | Ac-S5-Ala-Asp-Npg-B5-Asp-3COOHF-Aib-Ala-Phe-Lys*3-PyrS2-3Thi-34ClF-GlnR*3-NH2 |
| I-1233 | Ac-S5-Ala-Asp-Ile-B5-Asp-3COOHF-Aib-Ala-Phe-Lys*3-PyrS2-Phe-34ClF-GlnR*3-Ala-NH2 |
| I-1234 | Ac-S5-Ala-Asp-Ile-B5-Asp-3COOHF-Aib-Ala-Phe-Lys*3-PyrS2-Phe-34ClF-GlnR*3-NH2 |
| I-1235 | Ac-S5-Ala-Asp-Ala-B5-Asp-3COOHF-Aib-Ala-Phe-Lys*3-PyrS2-Phe-34ClF-GlnR*3-Ala-NH2 |
| I-1236 | Ac-S5-Ala-Asp-Ala-B5-Asp-3COOHF-Aib-Ala-Phe-Lys*3-PyrS2-Phe-34ClF-GlnR*3-NH2 |
| I-1237 | Ac-S5-Ala-Asp-Phe-B5-Asp-3COOHF-Aib-Ala-Phe-Lys*3-PyrS2-Phe-34ClF-GlnR*3-Ala-NH2 |
| I-1238 | Ac-S5-Ala-Asp-Phe-B5-Asp-3COOHF-Aib-Ala-Phe-Lys*3-PyrS2-Phe-34ClF-GlnR*3-NH2 |
| I-1239 | Ac-S5-Ala-Asp-Ile-B5-Asp-3COOHF-Ala-Ala-Phe-Lys*3-PyrS2-Phe-34ClF-GlnR*3-Ala-NH2 |
| I-1240 | Ac-S5-Ala-Asp-Ile-B5-Asp-3COOHF-Ala-Ala-Phe-Lys*3-PyrS2-Phe-34ClF-GlnR*3-NH2 |
| I-1241 | Ac-PL3-Asp-Ile-B5-Asp-3COOHF-[isovaleryl]Acp-Ala-Phe-TriAzLys*3-PyrS2-Phe-34ClF- |
| | sAla*3-NH2 |
| I-1242 | Ac-PL3-Asp-Npg-B5-Asp-3COOHF-Aib-Ala-Phe-Lys*3-PyrS2-Phe-BztA-GlnR*3-NH2 |
| I-1243 | Ac-PL3-Asp-Npg-B5-Asp-3COOHF-Ala-Ala-Phe-Lys*3-PyrS2-Phe-BztA-GlnR*3-NH2 |
| I-1244 | Ac-PL3-Asp-Npg-B5-Asp-3COOHF-Ala-Ala-Phe-Lys*3-PyrS2-Phe-BztA-GlnR*3-Ala-NH2 |
| I-1245 | Ac-PL3-Asp-Npg-B5-Asp-3COOHF-Aib-Ala-Phe-Lys*3-PyrS2-Phe-7FBztA-GlnR*3-NH2 |
| I-1246 | Ac-PL3-Asp-Npg-B5-Asp-3COOHF-Aib-Ala-Phe-Lys*3-PyrS2-Phe-7FBztA-GlnR*3-NH2 |
| I-1247 | Ac-PL3-Asp-Npg-B5-Asp-3COOHF-Aib-Ala-Phe-Lys*3-PyrS2-Phe-7FBztA-GlnR*3-Ala-NH2 |
| I-1248 | Ac-PL3-Asp-Npg-B5-Asp-3COOHF-Aib-Ala-Phe-Lys*3-PyrS2-Phe-7ClBztA-GlnR*3-NH2 |
| I-1249 | Ac-PL3-Asp-Npg-B5-Asp-3COOHF-Aib-Ala-Phe-Lys*3-PyrS2-Phe-7ClBztA-GlnR*3-Ala-NH2 |
| I-1250 | Ac-PL3-Asp-Npg-B5-Asp-3COOHF-Aib-Ala-Phe-Lys*3-PyrS2-Phe-7MeBztA-GlnR*3-NH2 |
| I-1251 | Ac-PL3-Asp-Npg-B5-Asp-3COOHF-Aib-Ala-Phe-Lys*3-PyrS2-Phe-7MeBztA-GlnR*3-Ala-NH2 |
| I-1252 | Ac-PL3-Asp-Ile-B5-Asp-3COOHF-Aib-Ala-Phe-Lys*3-PyrS2-Phe-34ClF-GlnR*3-Val-Glu-Ala- |
| | NH2 |
| I-1253 | Ac-PL3-Asp-Ile-B5-Asp-3COOHF-Aib-Ala-Phe-Lys*3-PyrS2-Phe-34ClF-GlnR*3-Val-Glu-Ala- |
| | NH2 |
| I-1254 | Ac-PL3-Asp-Ile-B5-Asp-3COOHF-Aib-Ala-Phe-Lys*3-PyrS2-Phe-34ClF-GlnR*3-Val-Glu-Leu- |
| | NH2 |
| I-1255 | Ac-PL3-Asp-Ile-B5-Asp-3COOHF-Aib-Ala-Phe-Lys*3-PyrS2-Phe-34ClF-GlnR*3-Val-Glu-Leu- |
| | NH2 |
| I-1256 | Ac-PL3-Asp-Ile-B5-Asp-3COOHF-Aib-Ala-Phe-Lys*3-PyrS2-Phe-34ClF-GlnR*3-Val-Thr-Ala- |
| | NH2 |
| I-1257 | Ac-PL3-Asp-Ile-B5-Asp-3COOHF-Aib-Ala-Phe-Lys*3-PyrS2-Phe-34ClF-GlnR*3-Val-Thr-Ala- |
| | NH2 |
| I-1258 | Ac-PL3-Asp-Ile-B5-Asp-3COOHF-CyLeu-Ala-Phe-Lys*3-PyrS2-Phe-34ClF-GlnR*3-Val-Glu-Ala- |
| | NH2 |
| I-1259 | Ac-PL3-Asp-Ile-B5-Asp-3COOHF-CyLeu-Ala-Phe-Lys*3-PyrS2-Phe-34ClF-GlnR*3-Val-Glu-Ala- |
| | NH2 |
| I-1260 | Ac-PL3-Asp-Ile-B5-Asp-3COOHF-CyLeu-Ala-Phe-Lys*3-PyrS2-Phe-34ClF-GlnR*3-Val-Glu-Leu- |
| | NH2 |
| I-1261 | Ac-PL3-Asp-Ile-B5-Asp-3COOHF-CyLeu-Ala-Phe-Lys*3-PyrS2-Phe-34ClF-GlnR*3-Val-Glu-Leu- |
| | NH2 |
| I-1262 | Ac-PL3-Asp-Ile-B5-Asp-3COOHF-CyLeu-Ala-Phe-Lys*3-PyrS2-Phe-34ClF-GlnR*3-Val-Thr-Ala- |
| | NH2 |
| I-1263 | Ac-PL3-Asp-Ile-B5-Asp-3COOHF-Aib-Ala-Phe-Lys*3-PyrS2-3Thi-34ClF-GlnR*3-Val-Glu-Ala- |
| | NH2 |
| I-1264 | Ac-PL3-Asp-Ile-B5-Asp-3COOHF-Aib-Ala-Phe-Lys*3-PyrS2-3Thi-34ClF-GlnR*3-Val-Glu-Leu- |
| | NH2 |
| I-1265 | Ac-PL3-Asp-Ile-B5-Asp-3COOHF-Aib-Ala-Phe-Lys*3-PyrS2-3Thi-34ClF-GlnR*3-Val-Thr-Ala- |
| | NH2 |
| I-1266 | Ac-PL3-Asp-Ile-B5-Asp-3COOHF-CyLeu-Ala-Phe-Lys*3-PyrS2-3Thi-34ClF-GlnR*3-Val-Glu- |
| | Ala-NH2 |
| I-1267 | Ac-PL3-Asp-Ile-B5-Asp-3COOHF-CyLeu-Ala-Phe-Lys*3-PyrS2-3Thi-34ClF-GlnR*3-Val-Glu- |
| | Leu-NH2 |
| I-1268 | Ac-PL3-Asp-Ile-B5-Asp-3COOHF-CyLeu-Ala-Phe-Lys*3-PyrS2-3Thi-34ClF-GlnR*3-Val-Thr- |
| | Ala-NH2 |
| I-1269 | Ac-PL3-Asp-Npg-B5-Asp-3COOHF-Aib-Ala-Phe-[3_3-biPh]hCys-PyrS2-3Thi-BztA-Cys-Ala-NH2 |
| I-1270 | Ac-PL3-Asp-Npg-B5-Asp-3COOHF-Aib-Ala-Phe-[2_6-naph]hCys-PyrS2-3Thi-BztA-Cys-Ala- |
| | NH2 |
| I-1271 | Ac-PL3-Asp-Npg-B5-Asp-3COOHF-Aib-Ala-Phe-[mPyr]Cys-PyrS2-3Thi-BztA-Cys-Ala-NH2 |
| I-1272 | Ac-PL3-Asp-Npg-B5-Asp-3COOHF-Aib-Ala-Phe-[mXyl]Cys-PyrS2-3Thi-BztA-Cys-Ala-NH2 |
| I-1273 | Ac-PL3-Asp-Npg-B5-Asp-3COOHF-Aib-Ala-Phe-[mPyr]hCys-PyrS2-3Thi-BztA-Cys-Ala-NH2 |
| I-1274 | Ac-PL3-Asp-Npg-B5-Asp-3COOHF-Aib-Ala-Phe-[C3]Cys-PyrS2-3Thi-BztA-Cys-Ala-NH2 |
| I-1275 | Ac-PL3-Asp-Npg-B5-Asp-3COOHF-Aib-Ala-Phe-[Red]Cys-PyrS2-3Thi-BztA-Cys-Ala-NH2 |
| I-1276 | Ac-PL3-Asp-Npg-B5-Asp-3COOHF-Aib-Ala-Phe-[33Oxe]hCys-PyrS2-3Thi-BztA-Cys-Ala-NH2 |
| I-1277 | Ac-PL3-Asp-Npg-B5-Asp-3COOHF-Aib-Ala-Phe-[33Oxe]Cys-PyrS2-3Thi-BztA-Cys-Ala-NH2 |
| I-1278 | Ac-PL3-Asp-Npg-B5-Asp-3COOHF-Aib-Ala-Phe-[IsoE]Cys-PyrS2-3Thi-BztA-Cys-Ala-NH2 |
| I-1279 | Ac-PL3-Asp-Npg-B5-Asp-3COOHF-Aib-Ala-Phe-[C3]hCys-PyrS2-3Thi-BztA-Cys-Ala-NH2 |
| I-1280 | Ac-PL3-Asp-Npg-B5-Asp-3COOHF-Aib-Ala-Phe-[Red]hCys-PyrS2-3Thi-BztA-Cys-Ala-NH2 |
| I-1281 | Ac-PL3-Asp-Npg-B5-Asp-3COOHF-Aib-Ala-Phe-[IsoE]hCys-PyrS2-3Thi-BztA-Cys-Ala-NH2 |
| I-1282 | Ac-PL3-Asp-Npg-B5-Asp-3COOHF-Aib-Ala-Phe-[13Ac]Cys-PyrS2-3Thi-BztA-Cys-Ala-NH2 |
| I-1283 | Ac-PL3-Asp-Npg-B5-Asp-3COOHF-Aib-Ala-Phe-[13Ac]hCys-PyrS2-3Thi-BztA-Cys-Ala-NH2 |
| I-1284 | Ac-Ala-Ala-S5-Ala-Asp-Npg-B5-Asp-3COOHF-Aib-Ala-Phe-Lys*3-PyrS2-3Thi-BztA-GlnR*3- |
| | Ala |
| I-1285 | Ac-Asp-Ala-S5-Ala-Asp-Npg-B5-Asp-3COOHF-Aib-Ala-Phe-Lys*3-PyrS2-3Thi-BztA-GlnR*3- |
| | Ala |
| I-1286 | Ac-Pro-Ala-S5-Ala-Asp-Npg-B5-Asp-3COOHF-Aib-Ala-Phe-Lys*3-PyrS2-3Thi-BztA-GlnR*3- |
| | Ala |
| I-1287 | Ac-Ala-Ala-S5-Ser-Asp-Npg-B5-Asp-3COOHF-Aib-Ala-Phe-Lys*3-PyrS2-3Thi-BztA-GlnR*3-Ala |
| I-1288 | Ac-Asp-Ala-S5-Ser-Asp-Npg-B5-Asp-3COOHF-Aib-Ala-Phe-Lys*3-PyrS2-3Thi-BztA-GlnR*3- |
| | Ala |
| I-1289 | Ac-Pro-Ala-S5-Ser-Asp-Npg-B5-Asp-3COOHF-Aib-Ala-Phe-Lys*3-PyrS2-3Thi-BztA-GlnR*3-Ala |
| I-1290 | Ac-Ala-Ala-S5-Leu-Asp-Npg-B5-Asp-3COOHF-Aib-Ala-Phe-Lys*3-PyrS2-3Thi-BztA-GlnR*3- |
| | Ala |
| I-1291 | Ac-Asp-Ala-S5-Leu-Asp-Npg-B5-Asp-3COOHF-Aib-Ala-Phe-Lys*3-PyrS2-3Thi-BztA-GlnR*3- |
| | Ala |
| I-1292 | Ac-Pro-Ala-S5-Leu-Asp-Npg-B5-Asp-3COOHF-Aib-Ala-Phe-Lys*3-PyrS2-3Thi-BztA-GlnR*3- |
| | Ala |
| I-1293 | Ac-Ala-Ser-S5-Ala-Asp-Npg-B5-Asp-3COOHF-Aib-Ala-Phe-Lys*3-PyrS2-3Thi-BztA-GlnR*3-Ala |
| I-1294 | Ac-Asp-Ser-S5-Ala-Asp-Npg-B5-Asp-3COOHF-Aib-Ala-Phe-Lys*3-PyrS2-3Thi-BztA-GlnR*3- |
| | Ala |
| I-1295 | Ac-Pro-Ser-S5-Ala-Asp-Npg-B5-Asp-3COOHF-Aib-Ala-Phe-Lys*3-PyrS2-3Thi-BztA-GlnR*3-Ala |
| I-1296 | Ac-S5-Leu-Asp-Ile-B5-Asp-3COOHF-Aib-Ala-Phe-Lys*3-PyrS2-Phe-34ClF-GlnR*3-NH2 |
| I-1297 | Ac-S5-Ala-Asp-Npg-B5-Ala-Asp-3COOHF-Aib-Ala-Phe-[4Abu]DapAc7-Leu-3Thi-BztA-GlnR*3- |
| | Ala-NH2 |
| I-1298 | Ac-S6-Ala-Asp-Npg-B5-Ala-Asp-3COOHF-Aib-Ala-Phe-[4Abu]DapAc7-Leu-3Thi-BztA-GlnR*3- |
| | Ala-NH2 |
| I-1299 | Ac-S5-Ala-Asp-Npg-B5-Asp-3COOHF-Aib-Ala-Phe-Leu-[4Abu]DapAc7-3thi-BztA-Ala-GlnR*3- |
| | NH2 |
| I-1300 | Ac-S6-Ala-Asp-Npg-B5-Asp-3COOHF-Aib-Ala-Phe-Leu-[4Abu]DapAc7-3thi-BztA-Ala-GlnR*3- |
| | NH2 |
| I-1301 | Ac-PL3-AspSH-Npg-B5-Asp-3COOHF-Aib-Ala-Phe-Lys*3-PyrS2-3Thi-BztA-GlnR*3-Ala-NH2 |
| I-1302 | Ac-PL3-Asp-Npg-B5-AspSH-3COOHF-Aib-Ala-Phe-Lys*3-PyrS2-3Thi-BztA-GlnR*3-Ala-NH2 |
| I-1303 | Ac-PL3-AspSH-Npg-B5-AspSH-3COOHF-Aib-Ala-Phe-Lys*3-PyrS2-3Thi-BztA-GlnR*3-Ala- |
| | NH2 |
| I-1304 | Ac-PL3-Asp-Npg-B5-Asp-3COOHF-Aib-Ala-Phe-Lys*3-PyrS2-hhLeu-BztA-GlnR*3-Ala-NH2 |
| I-1305 | Ac-PL3-Asp-Npg-B5-Asp-3COOHF-Aib-Ala-Phe-Lys*3-PyrS2-hPhe-BztA-GlnR*3-Ala-NH2 |
| I-1306 | Ac-PL3-Asp-Npg-B5-Asp-3COOHF-Aib-Ala-Phe-Lys*3-PyrS2-hhSer-BztA-GlnR*3-Ala-NH2 |
| I-1307 | Ac-PL3-Asp-Npg-B5-Asp-3COOHF-Aib-Ala-Phe-Lys*3-PyrS2-hhLeu-34ClF-GlnR*3-Ala-NH2 |
| I-1308 | Ac-PL3-Asp-Npg-B5-Asp-3COOHF-Aib-Ala-Phe-Lys*3-PyrS2-hPhe-34ClF-GlnR*3-Ala-NH2 |
| I-1309 | Ac-PL3-Asp-Npg-B5-Asp-3COOHF-Aib-Ala-Phe-Lys*3-PyrS2-hhSer-34ClF-GlnR*3-Ala-NH2 |
| I-1310 | Ac-PL3-Asp-Ile-B5-Asp-3COOHF-Aib-Ala-Phe-Lys*3-PyrS2-hhLeu-BztA-GlnR*3-Ala-NH2 |
| I-1311 | Ac-PL3-Asp-Ile-B5-Asp-3COOHF-Aib-Ala-Phe-Lys*3-PyrS2-hPhe-BztA-GlnR*3-Ala-NH2 |
| I-1312 | Ac-PL3-Asp-Ile-B5-Asp-3COOHF-Aib-Ala-Phe-Lys*3-PyrS2-hPhe-BztA-GlnR*3-Ala-NH2 |
| I-1313 | Ac-PL3-Asp-Ile-B5-Asp-3COOHF-Aib-Ala-Phe-Lys*3-PyrS2-hhSer-BztA-GlnR*3-Ala-NH2 |
| I-1314 | Ac-PL3-Asp-Ile-B5-Asp-3COOHF-Aib-Ala-Phe-Lys*3-PyrS2-hhLeu-34ClF-GlnR*3-Ala-NH2 |
| I-1315 | Ac-PL3-Asp-Ile-B5-Asp-3COOHF-Aib-Ala-Phe-Lys*3-PyrS2-hPhe-34ClF-GlnR*3-Ala-NH2 |
| I-1316 | Ac-PL3-Asp-Ile-B5-Asp-3COOHF-Aib-Ala-Phe-Lys*3-PyrS2-hhSer-34ClF-GlnR*3-Ala-NH2 |
| I-1317 | Ac-PL3-Asp-Npg-B5-Asp-3COOHF-Aib-Ala-Phe-Lys*3-PyrS2-hCbA-BztA-GlnR*3-Ala-NH2 |
| I-1318 | Ac-PL3-Asp-Npg-B5-Asp-3COOHF-Aib-Ala-Phe-Lys*3-PyrS2-hCypA-BztA-GlnR*3-Ala-NH2 |
| I-1319 | Ac-PL3-Asp-Npg-B5-Asp-3COOHF-Aib-Ala-Phe-Lys*3-PyrS2-hCha-BztA-GlnR*3-Ala-NH2 |
| I-1320 | Ac-PL3-Asp-Npg-B5-Asp-3COOHF-Aib-Ala-Phe-Lys*3-PyrS2-hCbA-34ClF-GlnR*3-Ala-NH2 |
| I-1321 | Ac-PL3-Asp-Npg-B5-Asp-3COOHF-Aib-Ala-Phe-Lys*3-PyrS2-hCypA-34ClF-GlnR*3-Ala-NH2 |
| I-1322 | Ac-PL3-Asp-Npg-B5-Asp-3COOHF-Aib-Ala-Phe-Lys*3-PyrS2-hCha-34ClF-GlnR*3-Ala-NH2 |
| I-1323 | Ac-PL3-Asp-Ile-B5-Asp-3COOHF-Aib-Ala-Phe-Lys*3-PyrS2-hCbA-BztA-GlnR*3-Ala-NH2 |
| I-1324 | Ac-PL3-Asp-Ile-B5-Asp-3COOHF-Aib-Ala-Phe-Lys*3-PyrS2-hCypA-BztA-GlnR*3-Ala-NH2 |
| I-1325 | Ac-PL3-Asp-Ile-B5-Asp-3COOHF-Aib-Ala-Phe-Lys*3-PyrS2-hCha-BztA-GlnR*3-Ala-NH2 |
| I-1326 | Ac-PL3-Asp-Ile-B5-Asp-3COOHF-Aib-Ala-Phe-Lys*3-PyrS2-hCbA-34ClF-GlnR*3-Ala-NH2 |
| I-1327 | Ac-PL3-Asp-Ile-B5-Asp-3COOHF-Aib-Ala-Phe-Lys*3-PyrS2-hCypA-34ClF-GlnR*3-Ala-NH2 |
| I-1328 | Ac-PL3-Asp-Ile-B5-Asp-3COOHF-Aib-Ala-Phe-Lys*3-PyrS2-hCha-34ClF-GlnR*3-Ala-NH2 |
| I-1329 | Ac-S5-Ala-Asp-Npg-B5-Asp-Asp-Aib-Ala-Phe-Lys*3-PyrS2-3Thi-BztA-GlnR*3-Ala-NH2 |
| I-1330 | Ac-S5-Ala-Asp-Npg-B5-Glu-Asp-Aib-Ala-Phe-Lys*3-PyrS2-3Thi-BztA-GlnR*3-Ala-NH2 |
| I-1331 | Ac-S5-Ala-Asp-Npg-B5-TfeGA-Asp-Aib-Ala-Phe-Lys*3-PyrS2-3Thi-BztA-GlnR*3-Ala-NH2 |
| I-1332 | Ac-S5-Pro-Asp-Npg-B5-Asp-Asp-Aib-Ala-Phe-Lys*3-PyrS2-3Thi-BztA-GlnR*3-Ala-NH2 |
| I-1333 | Ac-S5-Pro-Asp-Npg-B5-Glu-Asp-Aib-Ala-Phe-Lys*3-PyrS2-3Thi-BztA-GlnR*3-Ala-NH2 |
| I-1334 | Ac-S5-Pro-Asp-Npg-B5-TfeGA-Asp-Aib-Ala-Phe-Lys*3-PyrS2-3Thi-BztA-GlnR*3-Ala-NH2 |
| I-1335 | Ac-S5-Ala-Asp-Ile-B5-Asp-2COOHF-Aib-Ala-Phe-Lys*3-PyrS2-Phe-34ClF-GlnR*3-NH2 |
| I-1336 | Ac-S5-Ala-Asp-Ile-B5-Asp-4COOHF-Aib-Ala-Phe-Lys*3-PyrS2-Phe-34ClF-GlnR*3-NH2 |
| I-1337 | Ac-S5-Ala-Asp-Ile-B5-Asp-Asp-Aib-Ala-Phe-Lys*3-PyrS2-Phe-34ClF-GlnR*3-NH2 |
| I-1338 | Ac-S5-Ala-Asp-Ile-B5-Asp-Glu-Aib-Ala-Phe-Lys*3-PyrS2-Phe-34ClF-GlnR*3-NH2 |
| I-1339 | Ac-S5-Ala-Asp-Ile-B5-Asp-Asn-Aib-Ala-Phe-Lys*3-PyrS2-Phe-34ClF-GlnR*3-NH2 |
| I-1340 | Ac-S5-Ala-Asp-Ile-B5-Asp-Gln-Aib-Ala-Phe-Lys*3-PyrS2-Phe-34ClF-GlnR*3-NH2 |
| I-1341 | Ac-S5-Ala-Asp-Ile-B5-Asp-Ser-Aib-Ala-Phe-Lys*3-PyrS2-Phe-34ClF-GlnR*3-NH2 |
| I-1342 | Ac-S5-Tle-Asp-Ile-B5-Asp-3COOHF-Aib-Ala-Phe-Lys*3-PyrS2-Phe-34ClF-GlnR*3-NH2 |
| I-1343 | Ac-S5-Val-Asp-Ile-B5-Asp-3COOHF-Aib-Ala-Phe-Lys*3-PyrS2-Phe-34ClF-GlnR*3-NH2 |
| I-1344 | Ac-S5-Ile-Asp-Ile-B5-Asp-3COOHF-Aib-Ala-Phe-Lys*3-PyrS2-Phe-34ClF-GlnR*3-NH2 |
| I-1345 | Ac-PL3-Asp-Npg-B5-Asp-3COOHF-Aib-Ala-Phe-Lys*3-PyrS2-hCbA-BztA-GlnR*3-Val-Glu-Ala- |
| | NH2 |
| I-1346 | Ac-PL3-Asp-Npg-B5-Asp-3COOHF-Aib-Ala-Phe-Lys*3-PyrS2-hCypA-BztA-GlnR*3-Val-Glu- |
| | Ala-NH2 |
| I-1347 | Ac-PL3-Asp-Npg-B5-Asp-3COOHF-Aib-Ala-Phe-Lys*3-PyrS2-hCha-BztA-GlnR*3-Val-Glu-Ala- |
| | NH2 |
| I-1348 | Ac-PL3-Asp-Npg-B5-Asp-3COOHF-Aib-Ala-Phe-Lys*3-PyrS2-hCbA-34ClF-GlnR*3-Val-Glu- |
| | Ala-NH2 |
| I-1349 | Ac-PL3-Asp-Npg-B5-Asp-3COOHF-Aib-Ala-Phe-Lys*3-PyrS2-hCypA-34ClF-GlnR*3-Val-Glu- |
| | Ala-NH2 |
| I-1350 | Ac-PL3-Asp-Npg-B5-Asp-3COOHF-Aib-Ala-Phe-Lys*3-PyrS2-hCha-34ClF-GlnR*3-Val-Glu-Ala- |
| | NH2 |
| I-1351 | Ac-PL3-Asp-Ile-B5-Asp-3COOHF-Aib-Ala-Phe-Lys*3-PyrS2-Phe-34ClF-GlnR*3-Thr-Glu-Ala- |
| | NH2 |
| I-1352 | Ac-PL3-Asp-Ile-B5-Asp-3COOHF-Aib-Ala-Phe-Lys*3-PyrS2-Phe-34ClF-GlnR*3-Ser-Glu-Ala- |
| | NH2 |
| I-1353 | Ac-PL3-Asp-Ile-B5-Asp-3COOHF-Aib-Ala-Phe-Lys*3-PyrS2-Phe-34ClF-GlnR*3-Thr-Ser-Ala- |
| | NH2 |
| I-1354 | Ac-PL3-Asp-Ile-B5-Asp-3COOHF-Aib-Ala-Phe-Lys*3-PyrS2-Phe-34ClF-GlnR*3-Ser-Ser-Ala- |
| | NH2 |
| I-1355 | Ac-PL3-Asp-Ile-B5-Asp-3COOHF-Aib-Ala-Phe-Lys*3-PyrS2-Phe-34ClF-GlnR*3-Thr-Lys-Ala- |
| | NH2 |
| I-1356 | Ac-PL3-Asp-Ile-B5-Asp-3COOHF-Aib-Ala-Phe-Lys*3-PyrS2-Phe-34ClF-GlnR*3-Ser-Lys-Ala- |
| | NH2 |
| I-1357 | Ac-PL3-Asp-Ile-B5-Asp-3COOHF-Aib-Ala-Phe-Lys*3-PyrS2-Phe-34ClF-GlnR*3-Thr-Glu-Leu- |
| | NH2 |
| I-1358 | Ac-PL3-Asp-Ile-B5-Asp-3COOHF-Aib-Ala-Phe-Lys*3-PyrS2-Phe-34ClF-GlnR*3-Ser-Glu-Leu- |
| | NH2 |
| I-1359 | Ac-PL3-Asp-Ile-B5-Asp-3COOHF-Aib-Ala-Phe-Lys*3-PyrS2-Phe-34ClF-GlnR*3-Thr-Ser-Leu- |
| | NH2 |
| I-1360 | Ac-PL3-Asp-Ile-B5-Asp-3COOHF-Aib-Ala-Phe-Lys*3-PyrS2-Phe-34ClF-GlnR*3-Ser-Ser-Leu- |
| | NH2 |
| I-1361 | Ac-PL3-Asp-Ile-B5-Asp-3COOHF-Aib-Ala-Phe-Lys*3-PyrS2-Phe-34ClF-GlnR*3-Thr-Lys-Leu- |
| | NH2 |
| I-1362 | Ac-PL3-Asp-Ile-B5-Asp-3COOHF-Aib-Ala-Phe-Lys*3-PyrS2-Phe-34ClF-GlnR*3-Ser-Lys-Leu- |
| | NH2 |
| I-1363 | Ac-PL3-Asp-Npg-B5-Asp-3COOHF-Aib-Ala-Phe-Leu-[4Abu]DapAc7-3thi-BztA-Ala-GlnR-NH2 |
| I-1364 | Ac-PL3-Asp-Npg-B5-Asp-3COOHF-Aib-Ala-Phe-Leu-[SBut][CH2CH2NH]Dap-3thi-BztA-Ala- |
| | GlnR-NH2 |
| I-1365 | Ac-PL3-Asp-Npg-B5-Asp-3COOHF-Aib-Ala-Phe-[m5Meb]Cys-PyrS2-3Thi-BztA-Cys-Ala-NH2 |
| I-1366 | Ac-PL3-Asp-Npg-B5-Asp-3COOHF-Aib-Ala-Phe-[m5Meb]hCys-PyrS2-3Thi-BztA-Cys-Ala-NH2 |
| I-1367 | Ac-PL3-Asp-Ile-B5-Asp-3COOHF-Aib-Ala-Phe-GlnR*3-PyrS2-Phe-34ClF-Lys*3-NH2 |
| I-1368 | Ac-PL3-Asp-Ile-B5-Asp-3COOHF-Aib-Ala-Phe-GlnR*3-PyrS2-Phe-34ClF-Lys*3-NH2 |
| I-1369 | Ac-PL3-Asp-Ile-B5-Asp-3COOHF-Aib-Ala-Phe-1MeK*3-PyrS2-Phe-34ClF-GlnR*3-NH2 |
| I-1370 | Ac-PL3-Asp-Ile-B5-Asp-3COOHF-Aib-Ala-Phe-GlnR*3-PyrS2-Phe-34ClF-1MeK*3-NH2 |
| I-1371 | Ac-PL3-Asp-Ile-B5-Asp-3COOHF-Aib-Ala-Phe-Lys*3-PyrS2-Phe-34ClF-hGlnR*3-NH2 |
| I-1372 | Ac-PL3-Asp-Ile-B5-Asp-3COOHF-Aib-Ala-Phe-hGlnR*3-PyrS2-Phe-34ClF-Lys*3-NH2 |
| I-1373 | Ac-PL3-Asp-Ile-B5-Asp-3COOHF-Aib-Ala-Phe-1MeK*3-PyrS2-Phe-34ClF-hGlnR*3-NH2 |
| I-1374 | Ac-PL3-Asp-Ile-B5-Asp-3COOHF-Aib-Ala-Phe-hGlnR*3-PyrS2-Phe-34ClF-1MeK*3-NH2 |
| I-1375 | Ac-PL3-Asp-Ile-B5-Asp-3COOHF-Aib-Ala-Phe-hGlnR*3-PyrS2-Phe-34ClF-1MeK*3-NH2 |
| I-1376 | Ac-PL3-Asp-Ile-B5-Asp-3COOHF-Aib-Ala-Phe-GlnR*3-PyrS2-Phe-34ClF-AsnEDA*3-NH2 |
| I-1377 | Ac-PL3-Asp-Ile-B5-Asp-3COOHF-Aib-Ala-Phe-GlnR*3-PyrS2-Phe-34ClF-AsnEDA*3-NH2 |
| I-1378 | Ac-PL3-Asp-Ile-B5-Asp-3COOHF-Aib-Ala-Phe-GlnR*3-PyrS2-Phe-34ClF-GlnEDA*3-NH2 |
| I-1379 | Ac-PL3-Asp-Ile-B5-Asp-3COOHF-Aib-Ala-Phe-GlnR*3-PyrS2-Phe-34ClF-AsnPpz*3-NH2 |
| I-1380 | Ac-PL3-Asp-Ile-B5-Asp-3COOHF-Aib-Ala-Phe-GlnR*3-PyrS2-Phe-34ClF-GlnPpz*3-NH2 |
| I-1381 | Ac-PL3-Asp-Ile-B5-Asp-3COOHF-Aib-Ala-Phe-GlnR3APyr*3-PyrS2-Phe-34ClF-GlnR*3-NH2 |
| I-1382 | Ac-PL3-Asp-Ile-B5-Asp-3COOHF-Aib-Ala-Phe-GlnR*3-PyrS2-Phe-34ClF-GlnR3APyr*3-NH2 |
| I-1383 | Ac-PL3-Asp-Ile-B5-Asp-3COOHF-Aib-Ala-Phe-GlnS3APyr*3-PyrS2-Phe-34ClF-GlnR*3-NH2 |
| I-1384 | Ac-PL3-Asp-Ile-B5-Asp-3COOHF-Aib-Ala-Phe-GlnS3APyr*3-PyrS2-Phe-34ClF-GlnR*3-NH2 |
| I-1385 | Ac-PL3-Asp-Ile-B5-Asp-3COOHF-Aib-Ala-Phe-GlnR*3-PyrS2-Phe-34ClF-GlnS3APyr*3-NH2 |
| I-1386 | Ac-PL3-Asp-Npg-B5-Asp-3COOHF-Aib-Ala-Phe-[mXyl]Cys-PyrS2-3Thi-BztA-aMeC-Ala-NH2 |
| I-1387 | Ac-PL3-Asp-Npg-B5-Asp-3COOHF-Aib-Ala-Phe-[mPyr]Cys-PyrS2-3Thi-BztA-aMeC-Ala-NH2 |
| I-1388 | Ac-PL3-Asp-Npg-B5-Asp-3COOHF-Aib-Ala-Phe-[C3]Cys-PyrS2-3Thi-BztA-aMeC-Ala-NH2 |
| I-1389 | Ac-PL3-Asp-Npg-B5-Asp-3COOHF-Aib-Ala-Phe-[IsoE]Cys-PyrS2-3Thi-BztA-aMeC-Ala-NH2 |
| I-1390 | Ac-PL3-Asp-Npg-B5-Asp-3COOHF-Aib-Ala-Phe-[m5Meb]Cys-PyrS2-3Thi-BztA-aMeC-Ala-NH2 |
| I-1391 | Ac-PL3-Asp-Npg-B5-Asp-3COOHF-Aib-Ala-Phe-[33Oxe]Cys-PyrS2-3Thi-BztA-aMeC-Ala-NH2 |
| I-1392 | Ac-PL3-Asp-Npg-B5-Asp-3COOHF-Aib-Ala-Phe-Lys*3-PyrS2-3Thi-BztA-GlnR*3-Ala-Ala- |
| | [Ac]Lys-NH2 |
| I-1393 | Ac-PL3-Asp-Ile-B5-Asp-3COOHF-Aib-Ala-Phe-Lys*3-PyrS2-Phe-34ClF-GlnR*3-Ala-Ala- |
| | [Ac]Lys-NH2 |
| I-1394 | Ac-PL3-Asp-Npg-B5-Asp-3COOHF-Aib-Ala-Phe-Lys*3-PyrS2-3Thi-BztA-GlnR*3-Ala-Ala-Ala- |
| | [Ac]Lys-NH2 |
| I-1395 | Ac-PL3-Asp-Ile-B5-Asp-3COOHF-Aib-Ala-Phe-Lys*3-PyrS2-Phe-34ClF-GlnR*3-Ala-Ala-Ala- |
| | [Ac]Lys-NH2 |
| I-1396 | Ac-PL3-Asp-Npg-B5-Asp-[Ac]Dap-Aib-Ala-Phe-Lys*3-PyrS2-3Thi-BztA-GlnR*3-Ala-NH2 |
| I-1397 | Ac-PL3-Asp-Npg-B5-Asp-[CH2CO2H]Acp-Aib-Ala-Phe-Lys*3-PyrS2-3Thi-BztA-GlnR*3-Ala- |
| | NH2 |
| I-1398 | Ac-PL3-Asp-Npg-B5-Asp-[Pfbn]GA-Aib-Ala-Phe-Lys*3-PyrS2-3Thi-BztA-GlnR*3-Ala-NH2 |
| I-1399 | Ac-PL3-Asp-Npg-B5-Asp-[Tfb]GA-Aib-Ala-Phe-Lys*3-PyrS2-3Thi-BztA-GlnR*3-Ala-NH2 |
| I-1400 | Ac-PL3-Asp-Npg-B5-Asp-3COOHF-Aib-Ala-Phe-Lys*3-PyrS2-3Thi-BztA-GlnR*3-Ala-NHMe |
| I-1401 | Ac-PL3-Asp-Npg-B5-Asp-3COOHF-Aib-Ala-Phe-Lys*3-PyrS2-3Thi-BztA-GlnR*3-Ala-NHMe |
| I-1402 | Ac-PL3-Asp-Npg-B5-Asp-3COOHF-Aib-Ala-Phe-Lys*3-PyrS2-3Thi-BztA-GlnR*3-Ala-Ala- |
| | [mPEG4]Lys-NH2 |
| I-1403 | Ac-PL3-Asp-Npg-B5-Asp-3COOHF-Aib-Ala-Phe-Lys*3-PyrS2-3Thi-BztA-GlnR*3-Ala-Ala- |
| | [mPEG8]Lys-NH2 |
| I-1404 | Ac-PL3-Asp-Npg-B5-Asp-3COOHF-Aib-Ala-Phe-Lys*3-PyrS2-3Thi-BztA-GlnR*3-Ala-Ala- |
| | [mPEG16]Lys-NH2 |
| I-1405 | Ac-PL3-Asp-Ile-B5-Asp-3COOHF-Aib-Ala-Phe-Lys*3-PyrS2-Phe-34ClF-GlnR*3-Ala-Ala- |
| | [mPEG4]Lys-NH2 |
| I-1406 | Ac-PL3-Asp-Ile-B5-Asp-3COOHF-Aib-Ala-Phe-Lys*3-PyrS2-Phe-34ClF-GlnR*3-Ala-Ala- |
| | [mPEG8]Lys-NH2 |
| I-1407 | Ac-PL3-Asp-Ile-B5-Asp-3COOHF-Aib-Ala-Phe-Lys*3-PyrS2-Phe-34ClF-GlnR*3-Ala-Ala- |
| | [mPEG16]Lys-NH2 |
| I-1408 | Ac-PL3-Asp-Npg-B5-Asp-3COOHF-Aib-Ala-Phe-Lys*3-PyrS2-3Thi-BztA-GlnR*3-Ala-Ala-Ala- |
| | [mPEG8]Lys-NH2 |
| I-1409 | Ac-PL3-Asp-Npg-B5-Asp-3COOHF-Aib-Ala-Phe-Lys*3-PyrS2-3Thi-BztA-GlnR*3-Ala-Ala-Ala- |
| | [mPEG16]Lys-NH2 |
| I-1410 | Ac-PL3-Asp-Npg-B5-Asp-3COOHF-Aib-Ala-Phe-Lys*3-PyrS2-3Thi-BztA-GlnR*3-Ala-Ala-Ala- |
| | [mPEG37]Lys-NH2 |
| I-1411 | Ac-PL3-Asp-Ile-B5-Asp-3COOHF-Aib-Ala-Phe-Lys*3-PyrS2-Phe-34ClF-GlnR*3-Ala-Ala-Ala- |
| | [mPEG4]Lys-NH2 |
| I-1412 | Ac-PL3-Asp-Ile-B5-Asp-3COOHF-Aib-Ala-Phe-Lys*3-PyrS2-Phe-34ClF-GlnR*3-Ala-Ala-Ala- |
| | [mPEG8]Lys-NH2 |
| I-1413 | Ac-PL3-Asp-Ile-B5-Asp-3COOHF-Aib-Ala-Phe-Lys*3-PyrS2-Phe-34ClF-GlnR*3-Ala-Ala-Ala- |
| | [mPEG16]Lys-NH2 |
| I-1414 | Ac-PL3-Asp-Ile-B5-Asp-3COOHF-Aib-Ala-Phe-GlnR*3-PyrS2-Phe-34ClF-GlnMe2EDA*3-NH2 |
| I-1415 | Ac-PL3-Asp-Ile-B5-Asp-3COOHF-Aib-Ala-Phe-GlnR*3-PyrS2-Phe-34ClF-AsnMe2EDA*3-NH2 |
| I-1416 | Ac-PL3-Asp-Ile-B5-Asp-3COOHF-Aib-Ala-Phe-GlnR*3-PyrS2-Phe-34ClF-AsnMeEDA*3-NH2 |
| I-1417 | Ac-PL3-Asp-Ile-B5-Asp-3COOHF-Aib-Ala-Phe-AsnMeEDA*3-PyrS2-Phe-34ClF-GlnR*3-NH2 |
| I-1418 | Ac-PL3-Asp-Ile-B5-Asp-3COOHF-Aib-Ala-Phe-AsnMeEDA*3-PyrS2-Phe-34ClF-GlnR*3-NH2 |
| I-1419 | Ac-PL3-Asp-Ile-B5-Asp-3COOHF-Aib-Ala-Phe-GlnR*3-PyrS2-Phe-34ClF-AsnR3APyr*3-NH2 |
| I-1420 | Ac-PL3-Asp-Ile-B5-Asp-3COOHF-Aib-Ala-Phe-AsnR3APyr*3-PyrS2-Phe-34ClF-AsnR*3-NH2 |
| I-1421 | Ac-PL3-Asp-Ile-B5-Asp-3COOHF-Aib-Ala-Phe-AsnR3APyr*3-PyrS2-Phe-34ClF-GlnR*3-NH2 |
| I-1422 | Ac-PL3-Asp-Ile-B5-Asp-3COOHF-Aib-Ala-Phe-AsnR*3-PyrS2-Phe-34ClF-AsnS3APyr*3-NH2 |
| I-1423 | Ac-PL3-Asp-Ile-B5-Asp-3COOHF-Aib-Ala-Phe-GlnR*3-PyrS2-Phe-34ClF-AsnS3APyr*3-NH2 |
| I-1424 | Ac-PL3-Asp-Ile-B5-Asp-3COOHF-Aib-Ala-Phe-AsnS3APyr*3-PyrS2-Phe-34ClF-AsnR*3-NH2 |
| I-1425 | Ac-PL3-Asp-Npg-B5-Asp-3COOHF-Aib-Ala-Phe-[mPyr]hCys-PyrS2-3Thi-BztA-aMeC-Ala-NH2 |
| I-1426 | Ac-PL3-Asp-Npg-B5-Asp-3COOHF-Aib-Ala-Phe-[m5Meb]hCys-PyrS2-3Thi-BztA-aMeC-Ala- |
| | NH2 |
| I-1427 | Ac-PL3-Asp-Npg-B5-Asp-3COOHF-Aib-Ala-Phe-[C3]hCys-PyrS2-3Thi-BztA-aMeC-Ala-NH2 |
| I-1428 | Ac-PL3-Asp-Npg-B5-Asp-3COOHF-Aib-Ala-Phe-[Red]hCys-PyrS2-3Thi-BztA-aMeC-Ala-NH2 |
| I-1429 | Ac-PL3-Asp-Npg-B5-Asp-3COOHF-Aib-Ala-Phe-[IsoE]hCys-PyrS2-3Thi-BztA-aMeC-Ala-NH2 |
| I-1430 | Ac-PL3-Asp-Npg-B5-Asp-3COOHF-Aib-Ala-Phe-[13Ac]hCys-PyrS2-3Thi-BztA-aMeC-Ala-NH2 |
| I-1431 | Ac-PL3-Asp-Npg-B5-Asp-[Succinate]Dap-Aib-Ala-Phe-Lys*3-PyrS2-3Thi-BztA-GlnR*3-Ala-NH2 |
| I-1432 | Ac-PL3-Asp-Npg-B5-Asp-[Malonate]Dap-Aib-Ala-Phe-Lys*3-PyrS2-3Thi-BztA-GlnR*3-Ala-NH2 |
| I-1433 | Ac-PL3-Asp-Npg-B5-Asp-[Me2Mal]Dap-Aib-Ala-Phe-Lys*3-PyrS2-3Thi-BztA-GlnR*3-Ala-NH2 |
| I-1434 | Ac-PL3-Asp-Npg-B5-Asp-[SaiPrSuc]Dap-Aib-Ala-Phe-Lys*3-PyrS2-3Thi-BztA-GlnR*3-Ala-NH2 |
| I-1435 | Ac-PL3-Asp-Npg-B5-Asp-[SaMeSuc]Dap-Aib-Ala-Phe-Lys*3-PyrS2-3Thi-BztA-GlnR*3-Ala-NH2 |
| I-1436 | Ac-PL3-Asp-Npg-B5-Asp-[RaiPrSuc]Dap-Aib-Ala-Phe-Lys*3-PyrS2-3Thi-BztA-GlnR*3-Ala-NH2 |
| I-1437 | Ac-PL3-Asp-Npg-B5-Asp-3COOHF-Aib-Ala-Phe-TriAzLys*3-[4VinylBzt]PyrSa-3Thi-BztA- |
| | sAla*3-Ala-NH2 |
| I-1438 | Ac-PL3-Asp-Npg-B5-Asp-3COOHF-Aib-Ala-Phe-TriAzLys*3-[3OHBz]PyrSa-3Thi-BztA-sAla*3- |
| | Ala-NH2 |
| I-1439 | Ac-PL3-Asp-Npg-B5-Asp-3COOHF-Aib-Ala-Phe-TriAzLys*3-[3OHBz]PyrSa-3Thi-BztA-sAla*3- |
| | Ala-NH2 |
| I-1440 | Ac-PL3-Asp-Npg-B5-Asp-3COOHF-Aib-Ala-Phe-TriAzLys*3-[CO][Val]PyrSa-3Thi-BztA- |
| | sAla*3-Ala-NH2 |
| I-1441 | Ac-PL3-Asp-Npg-B5-Asp-3COOHF-Aib-Ala-Phe-TriAzLys*3-[CO][Val]PyrSa-3Thi-BztA- |
| | sAla*3-Ala-NH2 |
| I-1442 | Ac-PL3-Asp-Npg-B5-Asp-3COOHF-Aib-Ala-Phe-TriAzLys*3-[CO][dVal]PyrSa-3Thi-BztA- |
| | sAla*3-Ala-NH2 |
| I-1443 | Ac-PL3-Asp-Npg-B5-Asp-3COOHF-Aib-Ala-Phe-TriAzLys*3-[CO][Sar]PyrSa-3Thi-BztA-sAla*3- |
| | Ala-NH2 |
| I-1444 | Ac-PL3-Asp-Npg-B5-Asp-3COOHF-Aib-Ala-Phe-TriAzLys*3-[CO][Nip]PyrSa-3Thi-BztA- |
| | sAla*3-Ala-NH2 |
| I-1445 | Ac-PL3-Asp-Npg-B5-Asp-3COOHF-Aib-Ala-Phe-TriAzLys*3-[CO][dNip]PyrSa-3Thi-BztA- |
| | sAla*3-Ala-NH2 |
| I-1446 | Ac-PL3-Asp-Npg-B5-Asp-3COOHF-Aib-Ala-Phe-TriAzLys*3-[CO][dNip]PyrSa-3Thi-BztA- |
| | sAla*3-Ala-NH2 |
| I-1447 | Ac-PL3-Asp-Npg-B5-Asp-3COOHF-Aib-Ala-Phe-TriAzLys*3-[CO][dNip]PyrSa-3Thi-BztA- |
| | sAla*3-Ala-NH2 |
| I-1448 | Ac-PL3-Asp-Npg-B5-Asp-3COOHF-Aib-Ala-Phe-TriAzLys*3-[CO][Pro]PyrSa-3Thi-BztA- |
| | sAla*3-Ala-NH2 |
| I-1449 | Ac-PL3-Asp-Npg-B5-Asp-3COOHF-Aib-Ala-Phe-TriAzLys*3-[2Me4VinPhAc2]PyrSa-3Thi-BztA- |
| | sAla*3-Ala-NH2 |
| I-1450 | Ac-PL3-Asp-Npg-B5-Asp-3COOHF-Aib-Ala-Phe-TriAzLys*3-[3SBz]PyrSa-3Thi-BztA-sAla*3- |
| | Ala-NH2 |
| I-1451 | Ac-PL3-Asp-Npg-B5-Asp-3COOHF-Aib-Ala-Phe-[m5Pyr]Cys-PyrS2-3Thi-BztA-Cys-Ala-NH2 |
| I-1452 | Ac-S6-Pro-Asp-Npg-B5-Asp-3COOHF-Aib-Ala-Phe-Lys*3-PyrS2-Phe-34ClF-GlnR*3-NH2 |
| I-1453 | Ac-PL3-Asp-Npg-B5-Asp-3COOHF-Aib-Ala-Phe-[m50Meb]Cys-PyrS2-3Thi-BztA-Cys-Ala-NH2 |
| I-1454 | Ac-PL3-Asp-Npg-B5-Asp-3COOHF-Aib-Ala-Phe-[mPyr]Cys-PyrS2-3Thi-BztA-Pen-Ala-NH2 |
| I-1455 | Ac-PL3-Asp-Npg-B5-Asp-3COOHF-Aib-Ala-Phe-[m5Pyr]Cys-PyrS2-3Thi-BztA-Pen-Ala-NH2 |
| I-1456 | Ac-PL3-Asp-Npg-B5-Asp-3COOHF-Aib-Ala-Phe-[m50Meb]Cys-PyrS2-3Thi-BztA-Pen-Ala-NH2 |
| I-1457 | Ac-PL3-Asp-Npg-B5-Asp-3COOHF-Aib-Ala-Phe-[Red]Cys-PyrS2-3Thi-BztA-Pen-Ala-NH2 |
| I-1458 | Ac-PL3-Asp-Npg-B5-Asp-3COOHF-Aib-Ala-Phe-[IsoE]Cys-PyrS2-3Thi-BztA-Pen-Ala-NH2 |
| I-1459 | Ac-PL3-Asp-Npg-B5-Asp-3COOHF-Aib-Ala-Phe-[C3]Cys-PyrS2-3Thi-BztA-Pen-Ala-NH2 |
| I-1460 | Ac-PL3-Asp-Npg-B5-Asp-3COOHF-Aib-Ala-Phe-[IsoE]hCysOx-PyrS2-3Thi-BztA-aMeC-Ala- |
| | NH2 |
| I-1461 | Ac-PL3-Asp-Npg-B5-Asp-3COOHF-Aib-Ala-Phe-[mPyr]Cys-PyrS2-3Thi-BztA-hCys-Ala-NH2 |
| I-1462 | Ac-PL3-Asp-Npg-B5-Asp-3COOHF-Aib-Ala-Phe-[IsoE]Cys-PyrS2-3Thi-BztA-hCys-Ala-NH2 |
| I-1463 | Ac-S5-Glu-Asp-Npg-B5-Asp-3COOHF-Aib-Ala-Phe-Lys*3-PyrS2-Phe-34ClF-GlnR*3-NH2 |
| I-1464 | Ac-S6-Val-Asp-Npg-B5-Asp-3COOHF-Aib-Ala-Phe-Lys*3-PyrS2-Phe-34ClF-GlnR*3-NH2 |
| I-1465 | Ac-S5-Ala-Asp-Npg-B5-Asp-3COOHF-Aib-Ala-Phe-Lys*3-PyrS2-Phe-34ClF-GlnR*3-NH2 |
| I-1466 | Ac-S5-Val-Asp-Npg-B5-Asp-3COOHF-Aib-Ala-Phe-Lys*3-PyrS2-Phe-34ClF-GlnR*3-NH2 |
| I-1467 | Ac-S6-Glu-Asp-Npg-B5-Asp-3COOHF-Aib-Ala-Phe-Lys*3-PyrS2-Phe-34ClF-GlnR*3-NH2 |
| I-1468 | Ac-S6-Ala-Asp-Npg-B5-Asp-3COOHF-Aib-Ala-Phe-Lys*3-PyrS2-Phe-34ClF-GlnR*3-NH2 |
| I-1469 | Ac-PL3-Asp-Npg-B5-Asp-3COOHF-Aib-Ala-Phe-[mPyr]hCys-PyrS2-3Thi-BztA-Pen-Ala-NH2 |
| I-1470 | Ac-PL3-Asp-Npg-B5-Asp-3COOHF-Aib-Ala-Phe-[m5Pyr]hCys-PyrS2-3Thi-BztA-Pen-Ala-NH2 |
| I-1471 | Ac-PL3-Asp-Npg-B5-Asp-3COOHF-Aib-Ala-Phe-[C3]hCys-PyrS2-3Thi-BztA-Pen-Ala-NH2 |
| I-1472 | Ac-PL3-Asp-Npg-B5-Asp-3COOHF-Aib-Ala-Phe-Lys*3-PyrS2-3Thi-BztA-GlnR*3-Thr-Ser-Ala- |
| | NH2 |
| I-1473 | Ac-PL3-Asp-Npg-B5-Asp-3COOHF-Aib-Ala-Phe-Lys*3-PyrS2-3Thi-BztA-GlnR*3-Thr-Ser-Leu- |
| | NH2 |
| I-1474 | Ac-PL3-Asp-Npg-B5-Asp-3COOHF-Aib-Ala-Phe-Lys*3-PyrS2-3Thi-BztA-GlnR*3-Thr-Ser-Leu- |
| | Pro-NH2 |
| I-1475 | Ac-PL3-Asp-Npg-B5-Asp-3COOHF-Aib-Ala-Phe-Lys*3-PyrS2-3Thi-BztA-GlnR*3-Thr-Ser-Thr- |
| | NH2 |
| I-1476 | Ac-PL3-Asp-Npg-B5-Asp-3COOHF-Aib-Ala-Phe-Lys*3-PyrS2-3Thi-BztA-GlnR*3-Thr-Ser-Thr- |
| | Pro-NH2 |
| I-1477 | Ac-PL3-Asp-Npg-B5-Asp-3COOHF-Aib-Ala-Phe-Lys*3-PyrS2-3Thi-BztA-GlnR*3-Thr-Ser-Pro- |
| | NH2 |
| I-1478 | Ac-PL3-Asp-Npg-B5-Asp-3COOHF-Aib-Ala-Phe-Lys*3-PyrS2-3Thi-BztA-GlnR*3-Val-Ser-Ala- |
| | NH2 |
| I-1479 | Ac-PL3-Asp-Npg-B5-Asp-3COOHF-Aib-Ala-Phe-Lys*3-PyrS2-3Thi-BztA-GlnR*3-Val-Ser-Leu- |
| | NH2 |
| I-1480 | Ac-PL3-Asp-Npg-B5-Asp-3COOHF-Aib-Ala-Phe-Lys*3-PyrS2-3Thi-BztA-GlnR*3-Val-Ser-Leu- |
| | Pro-NH2 |
| I-1481 | Ac-PL3-Asp-Npg-B5-Asp-3COOHF-Aib-Ala-Phe-Lys*3-PyrS2-3Thi-BztA-GlnR*3-Val-Ser-Thr- |
| | NH2 |
| I-1482 | Ac-PL3-Asp-Npg-B5-Asp-3COOHF-Aib-Ala-Phe-Lys*3-PyrS2-3Thi-BztA-GlnR*3-Val-Ser-Thr- |
| | Pro-NH2 |
| I-1483 | Ac-PL3-Asp-Npg-B5-Asp-3COOHF-Aib-Ala-Phe-Lys*3-PyrS2-3Thi-BztA-GlnR*3-Val-Ser-Pro- |
| | NH2 |
| I-1484 | Ac-PL3-Asp-R5-S5-Asp-3COOHF-Aib-Ala-Phe-PyrS2-Lys*3-3Thi-BztA-Ala-GlnR*3-NH2 |
| I-1485 | Ac-PL3-Asp-R5-S5-Asp-3COOHF-Aib-Ala-Phe-PyrS2-Lys*3-3Thi-BztA-Ala-GlnR*3-NH2 |
| I-1486 | NPyroR3-Asp-Npg-B4-Asp-3COOHF-Aib-Ala-Phe-Lys*3-PyrS3-3Thi-BztA-GlnR*3-Ala-NH2 |
| I-1487 | NPyroR3-Asp-Npg-B4-Asp-3COOHF-Aib-Ala-Phe-Lys*3-PyrS3-3Thi-BztA-GlnR*3-Ala-NH2 |
| I-1488 | NPyroR3-Asp-Npg-B5-Asp-3COOHF-Aib-Ala-Phe-Lys*3-PyrS2-3Thi-BztA-GlnR*3-Ala-NH2 |
| I-1489 | NPyroR3-Asp-Npg-B5-Asp-3COOHF-Aib-Ala-Phe-Lys*3-PyrS2-3Thi-BztA-GlnR*3-Ala-NH2 |
| I-1490 | NPyroR3-Asp-Npg-B6-Asp-3COOHF-Aib-Ala-Phe-Lys*3-PyrS1-3Thi-BztA-GlnR*3-Ala-NH2 |
| I-1491 | NPyroR3-Asp-Npg-B6-Asp-3COOHF-Aib-Ala-Phe-Lys*3-PyrS1-3Thi-BztA-GlnR*3-Ala-NH2 |
| I-1492 | NPyroR3-Asp-Ala-B4-Asp-3COOHF-Aib-Ala-Phe-Lys*3-PyrS3-3Thi-BztA-GlnR*3-Ala-NH2 |
| I-1493 | NPyroR3-Asp-Ala-B5-Asp-3COOHF-Aib-Ala-Phe-Lys*3-PyrS2-3Thi-BztA-GlnR*3-Ala-NH2 |
| I-1494 | NPyroR3-Asp-Ala-B6-Asp-3COOHF-Aib-Ala-Phe-Lys*3-PyrS1-3Thi-BztA-GlnR*3-Ala-NH2 |
| I-1495 | NPyroR3-Asp-Ala-B6-Asp-3COOHF-Aib-Ala-Phe-Lys*3-PyrS1-3Thi-BztA-GlnR*3-Ala-NH2 |
| I-1496 | NPyroR3-Asp-Npg-B4-Asp-3COOHF-Aib-Ala-Phe-Lys*3-PyrS3-3Thi-BztA-GlnR*3-Ala-NH2 |
| I-1497 | NPyroR3-Asp-Npg-B5-Asp-3COOHF-Aib-Ala-Phe-Lys*3-PyrS2-3Thi-BztA-GlnR*3-Ala-NH2 |
| I-1498 | NPyroR3-Asp-Npg-B6-Asp-3COOHF-Aib-Ala-Phe-Lys*3-PyrS1-3Thi-BztA-GlnR*3-Ala-NH2 |
| I-1499 | NPyroR3-Asp-Ala-B4-Asp-3COOHF-Aib-Ala-Phe-Lys*3-PyrS3-3Thi-BztA-GlnR*3-Ala-NH2 |
| I-1500 | NPyroR3-Asp-Ala-B5-Asp-3COOHF-Aib-Ala-Phe-Lys*3-PyrS2-3Thi-BztA-GlnR*3-Ala-NH2 |
| I-1501 | NPyroR3-Asp-Ala-B6-Asp-3COOHF-Aib-Ala-Phe-Lys*3-PyrS1-3Thi-BztA-GlnR*3-Ala-NH2 |
| I-1502 | Ac-PL3-Asp-Cha-B5-Asp-3COOHF-Aib-Ala-Phe-Lys*3-PyrS2-2ClF-34ClF-GlnR*3-NH2 |
| I-1503 | Ac-PL3-Asp-Cha-B5-Asp-3COOHF-Ala-Ala-Phe-Lys*3-PyrS2-2ClF-34ClF-GlnR*3-NH2 |
| I-1504 | Ac-PL3-Asp-Cha-B5-Asp-3COOHF-CyLeu-Ala-Phe-Lys*3-PyrS2-2ClF-34ClF-GlnR*3-NH2 |
| I-1505 | Ac-PL3-Asp-Chg-B5-Asp-3COOHF-Aib-Ala-Phe-Lys*3-PyrS2-2ClF-34ClF-GlnR*3-NH2 |
| I-1506 | Ac-PL3-Asp-Chg-B5-Asp-3COOHF-Ala-Ala-Phe-Lys*3-PyrS2-2ClF-34ClF-GlnR*3-NH2 |
| I-1507 | Ac-PL3-Asp-Chg-B5-Asp-3COOHF-CyLeu-Ala-Phe-Lys*3-PyrS2-2ClF-34ClF-GlnR*3-NH2 |
| I-1508 | Ac-PL3-Asp-Ile-B5-Asp-3COOHF-Aib-Ala-Phe-Lys*3-PyrS2-2MeF-34ClF-GlnR*3-NH2 |
| I-1509 | Ac-PL3-Asp-Ile-B5-Asp-3COOHF-Aib-Ala-Phe-Lys*3-PyrS2-2MeF-34ClF-GlnR*3-NH2 |
| I-1510 | Ac-PL3-Asp-Ile-B5-Asp-3COOHF-Ala-Ala-Phe-Lys*3-PyrS2-2MeF-34ClF-GlnR*3-NH2 |
| I-1511 | Ac-PL3-Asp-Ile-B5-Asp-3COOHF-CyLeu-Ala-Phe-Lys*3-PyrS2-2MeF-34ClF-GlnR*3-NH2 |
| I-1512 | Ac-PL3-Asp-Leu-B5-Asp-3COOHF-Aib-Ala-Phe-Lys*3-PyrS2-2MeF-34ClF-GlnR*3-NH2 |
| I-1513 | Ac-PL3-Asp-Leu-B5-Asp-3COOHF-Ala-Ala-Phe-Lys*3-PyrS2-2MeF-34ClF-GlnR*3-NH2 |
| I-1514 | Ac-PL3-Asp-Leu-B5-Asp-3COOHF-CyLeu-Ala-Phe-Lys*3-PyrS2-2MeF-34ClF-GlnR*3-NH2 |
| I-1515 | Ac-PL3-Asp-Cha-B5-Asp-3COOHF-Aib-Ala-Phe-Lys*3-PyrS2-2MeF-34ClF-GlnR*3-NH2 |
| I-1516 | Ac-PL3-Asp-Cha-B5-Asp-3COOHF-Ala-Ala-Phe-Lys*3-PyrS2-2MeF-34ClF-GlnR*3-NH2 |
| I-1517 | Ac-PL3-Asp-Chg-B5-Asp-3COOHF-Aib-Ala-Phe-Lys*3-PyrS2-2MeF-34ClF-GlnR*3-NH2 |
| I-1518 | Ac-PL3-Asp-Chg-B5-Asp-3COOHF-Ala-Ala-Phe-Lys*3-PyrS2-2MeF-34ClF-GlnR*3-NH2 |
| I-1519 | Ac-PL3-Asp-Npg-B5-Asp-3COOHF-Aib-Ala-Phe-[Mxyl]Cys-PyrS2-3Thi-BztA-Cys-Ser-NH2 |
| I-1520 | Ac-PL3-Asp-Npg-B5-Asp-3COOHF-Aib-Ala-Phe-[Mpyr]Cys-PyrS2-3Thi-BztA-Cys-Ser-NH2 |
| I-1521 | Ac-PL3-Asp-Npg-B5-Asp-3COOHF-Aib-Ala-Phe-[Red]Cys-PyrS2-3Thi-BztA-Cys-Ser-NH2 |
| I-1522 | Ac-PL3-Asp-Npg-B5-Asp-3COOHF-Aib-Ala-Phe-[C3]Cys-PyrS2-3Thi-BztA-Cys-Ser-NH2 |
| I-1523 | Ac-S6-Aib-Asp-Npg-B5-Asp-3COOHF-Aib-Ala-Phe-Lys*3-PyrS2-Phe-34ClF-GlnR*3-NH2 |
| I-1524 | Ac-S6-MePro-Asp-Npg-B5-Asp-3COOHF-Aib-Ala-Phe-Lys*3-PyrS2-Phe-34ClF-GlnR*3-NH2 |
| I-1525 | Ac-PL3-Asp-Npg-B5-Asp-3COOHF-Aib-Ala-Phe-Lys*3-PyrS2-3Thi-BztA-GlnR*3-Ala-Ala-Ala- |
| | [PEG4triPEG16]Lys-NH2 |
| I-1526 | Ac-PL3-Asp-Npg-B5-Asp-3COOHF-Aib-Ala-Phe-Lys*3-PyrS2-3Thi-BztA-GlnR*3-Ala-Ala-Ala- |
| | [PEG4triPEG36]Lys-NH2 |
| I-1527 | Ac-PL3-Asp-Cha-B5-Asp-3COOHF-Aib-Ala-Phe-Lys*3-PyrS2-2ClF-BztA-GlnR*3-NH2 |
| I-1528 | Ac-PL3-Asp-Cha-B5-Asp-3COOHF-Ala-Ala-Phe-Lys*3-PyrS2-2ClF-BztA-GlnR*3-NH2 |
| I-1529 | Ac-PL3-Asp-Cha-B5-Asp-3COOHF-CyLeu-Ala-Phe-Lys*3-PyrS2-2ClF-BztA-GlnR*3-NH2 |
| I-1530 | Ac-PL3-Asp-Chg-B5-Asp-3COOHF-Aib-Ala-Phe-Lys*3-PyrS2-2ClF-BztA-GlnR*3-NH2 |
| I-1531 | Ac-PL3-Asp-Chg-B5-Asp-3COOHF-Ala-Ala-Phe-Lys*3-PyrS2-2ClF-BztA-GlnR*3-NH2 |
| I-1532 | Ac-PL3-Asp-Npg-B5-Asp-3COOHF-Aib-Ala-Phe-Lys*3-PyrS2-DipA-34ClF-GlnR*3-NH2 |
| I-1533 | Ac-PL3-Asp-Npg-B5-Asp-3COOHF-Aib-Ala-Phe-Lys*3-PyrS2-DipA-34ClF-GlnR*3-NH2 |
| I-1534 | Ac-PL3-Asp-Ile-B5-Asp-3COOHF-Aib-Ala-Phe-Lys*3-PyrS2-DipA-34ClF-GlnR*3-NH2 |
| I-1535 | Ac-PL3-Asp-Ile-B5-Asp-3COOHF-Aib-Ala-Phe-Lys*3-PyrS2-DipA-34ClF-GlnR*3-NH2 |
| I-1536 | Ac-PL3-Asp-Npg-B5-Asp-3COOHF-Aib-Ala-Phe-Lys*3-PyrS2-DipA-BztA-GlnR*3-NH2 |
| I-1537 | Ac-PL3-Asp-Npg-B5-Asp-3COOHF-Aib-Ala-Phe-Lys*3-PyrS2-2FF-34ClF-GlnR*3-NH2 |
| I-1538 | Ac-PL3-Asp-Npg-B5-Asp-3COOHF-Aib-Ala-Phe-Lys*3-PyrS2-2FF-34ClF-GlnR*3-NH2 |
| I-1539 | Ac-PL3-Asp-Ile-B5-Asp-3COOHF-Aib-Ala-Phe-Lys*3-PyrS2-2FF-34ClF-GlnR*3-NH2 |
| I-1540 | Ac-PL3-Asp-Ile-B5-Asp-3COOHF-Aib-Ala-Phe-Lys*3-PyrS2-2FF-34ClF-GlnR*3-NH2 |
| I-1541 | Ac-PL3-Asp-Npg-B5-Asp-3COOHF-Aib-Ala-Phe-Lys*3-PyrS2-2FF-BztA-GlnR*3-NH2 |
| I-1542 | Ac-PL3-Asp-Npg-B5-Asp-3COOHF-Aib-Ala-Phe-Lys*3-PyrS2-2FF-BztA-GlnR*3-NH2 |
| I-1543 | Ac-PL3-Asp-Ile-B5-Asp-3COOHF-Aib-Ala-Phe-Lys*3-PyrS2-2FF-BztA-GlnR*3-NH2 |
| I-1544 | Ac-PL3-Asp-Ile-B5-Asp-3COOHF-Aib-Ala-Phe-Lys*3-PyrS2-2FF-BztA-GlnR*3-NH2 |
| I-1545 | Ac-PL3-Asp-Npg-B5-Asp-3COOHF-Aib-Ala-Phe-Lys*3-PyrS2-2MeF-BztA-GlnR*3-NH2 |
| I-1546 | Ac-PL3-Asp-Ile-B5-Asp-3COOHF-Aib-Ala-Phe-Lys*3-PyrS2-2MeF-BztA-GlnR*3-NH2 |
| I-1547 | Ac-PL3-Asp-Ile-B5-Asp-3COOHF-Aib-Ala-Phe-Lys*3-PyrS2-2MeF-BztA-GlnR*3-NH2 |
| I-1548 | NPyroR3-Asp-Npg-B5-Asp-3COOHF-Aib-Ala-Phe-Lys*3-PyrS2-3Thi-34ClF-GlnR*3-NH2 |
| I-1549 | NPyroR3-Asp-Ala-B5-Asp-3COOHF-Aib-Ala-Phe-Lys*3-PyrS2-3Thi-34ClF-GlnR*3-NH2 |
| I-1550 | NPyroR3-Asp-Ile-B5-Asp-3COOHF-Aib-Ala-Phe-Lys*3-PyrS2-3Thi-34ClF-GlnR*3-NH2 |
| I-1551 | NPyroR3-Asp-Val-B5-Asp-3COOHF-Aib-Ala-Phe-Lys*3-PyrS2-3Thi-34ClF-GlnR*3-NH2 |
| I-1552 | NPyroR3-Asp-Leu-B5-Asp-3COOHF-Aib-Ala-Phe-Lys*3-PyrS2-3Thi-34ClF-GlnR*3-NH2 |
| I-1553 | NPyroR3-Asp-Phe-B5-Asp-3COOHF-Aib-Ala-Phe-Lys*3-PyrS2-3Thi-34ClF-GlnR*3-NH2 |
| I-1554 | NPyroR3-Asp-Chg-B5-Asp-3COOHF-Aib-Ala-Phe-Lys*3-PyrS2-3Thi-34ClF-GlnR*3-NH2 |
| I-1555 | NPyroR3-Asp-Cha-B5-Asp-3COOHF-Aib-Ala-Phe-Lys*3-PyrS2-3Thi-34ClF-GlnR*3-NH2 |
| I-1556 | NPyroR3-Asp-DipA-B5-Asp-3COOHF-Aib-Ala-Phe-Lys*3-PyrS2-3Thi-34ClF-GlnR*3-NH2 |
| I-1557 | NPyroR3-Asn-Npg-B5-Asp-3COOHF-Aib-Ala-Phe-Lys*3-PyrS2-3Thi-34ClF-GlnR*3-NH2 |
| I-1558 | NPyroR3-Ser-Npg-B5-Asp-3COOHF-Aib-Ala-Phe-Lys*3-PyrS2-3Thi-34ClF-GlnR*3-NH2 |
| I-1559 | NPyroR3-Asp-Npg-B5-Asp-3COOHF-Ala-Ala-Phe-Lys*3-PyrS2-3Thi-34ClF-GlnR*3-NH2 |
| I-1560 | C3a-PL3-Asp-Npg-B5-Asp-3COOHF-Aib-Ala-Phe-Lys*3-PyrS2-Phe-34ClF-GlnR*3-NH2 |
| I-1561 | C3a-PL3-Asp-Npg-B5-Asp-3COOHF-Aib-Ala-Phe-Lys*3-PyrS2-Phe-34ClF-GlnR*3-NH2 |
| I-1562 | Bua-PL3-Asp-Npg-B5-Asp-3COOHF-Aib-Ala-Phe-Lys*3-PyrS2-Phe-34ClF-GlnR*3-NH2 |
| I-1563 | isobutyryl-PL3-Asp-Npg-B5-Asp-3COOHF-Aib-Ala-Phe-Lys*3-PyrS2-Phe-34ClF-GlnR*3-NH2 |
| I-1564 | Cpc-PL3-Asp-Npg-B5-Asp-3COOHF-Aib-Ala-Phe-Lys*3-PyrS2-Phe-34ClF-GlnR*3-NH2 |
| I-1565 | Cpc-PL3-Asp-Npg-B5-Asp-3COOHF-Aib-Ala-Phe-Lys*3-PyrS2-Phe-34ClF-GlnR*3-NH2 |
| I-1566 | Cbc-PL3-Asp-Npg-B5-Asp-3COOHF-Aib-Ala-Phe-Lys*3-PyrS2-Phe-34ClF-GlnR*3-NH2 |
| I-1567 | Cbc-PL3-Asp-Npg-B5-Asp-3COOHF-Aib-Ala-Phe-Lys*3-PyrS2-Phe-34ClF-GlnR*3-NH2 |
| I-1568 | CypCO-PL3-Asp-Npg-B5-Asp-3COOHF-Aib-Ala-Phe-Lys*3-PyrS2-Phe-34ClF-GlnR*3-NH2 |
| I-1569 | CypCO-PL3-Asp-Npg-B5-Asp-3COOHF-Aib-Ala-Phe-Lys*3-PyrS2-Phe-34ClF-GlnR*3-NH2 |
| I-1570 | 4THPCO-PL3-Asp-Npg-B5-Asp-3COOHF-Aib-Ala-Phe-Lys*3-PyrS2-Phe-34ClF-GlnR*3-NH2 |
| I-1571 | 4THPCO-PL3-Asp-Npg-B5-Asp-3COOHF-Aib-Ala-Phe-Lys*3-PyrS2-Phe-34ClF-GlnR*3-NH2 |
| I-1572 | C3a-PL3-Asp-Cha-B5-Asp-3COOHF-Aib-Ala-Phe-Lys*3-PyrS2-3Thi-34ClF-GlnR*3-NH2 |
| I-1573 | C3a-PL3-Asp-Cha-B5-Asp-3COOHF-Aib-Ala-Phe-Lys*3-PyrS2-3Thi-34ClF-GlnR*3-NH2 |
| I-1574 | Bua-PL3-Asp-Cha-B5-Asp-3COOHF-Aib-Ala-Phe-Lys*3-PyrS2-3Thi-34ClF-GlnR*3-NH2 |
| I-1575 | Bua-PL3-Asp-Cha-B5-Asp-3COOHF-Aib-Ala-Phe-Lys*3-PyrS2-3Thi-34ClF-GlnR*3-NH2 |
| I-1576 | isobutyryl-PL3-Asp-Cha-B5-Asp-3COOHF-Aib-Ala-Phe-Lys*3-PyrS2-3Thi-34ClF-GlnR*3-NH2 |
| I-1577 | Cpc-PL3-Asp-Cha-B5-Asp-3COOHF-Aib-Ala-Phe-Lys*3-PyrS2-3Thi-34ClF-GlnR*3-NH2 |
| I-1578 | Cpc-PL3-Asp-Cha-B5-Asp-3COOHF-Aib-Ala-Phe-Lys*3-PyrS2-3Thi-34ClF-GlnR*3-NH2 |
| I-1579 | Cbc-PL3-Asp-Cha-B5-Asp-3COOHF-Aib-Ala-Phe-Lys*3-PyrS2-3Thi-34ClF-GlnR*3-NH2 |
| I-1580 | CypCO-PL3-Asp-Cha-B5-Asp-3COOHF-Aib-Ala-Phe-Lys*3-PyrS2-3Thi-34ClF-GlnR*3-NH2 |
| I-1581 | 4THPCO-PL3-Asp-Cha-B5-Asp-3COOHF-Aib-Ala-Phe-Lys*3-PyrS2-3Thi-34ClF-GlnR*3-NH2 |
| I-1582 | C3a-PL3-Asp-Ile-B5-Asp-3COOHF-Aib-Ala-Phe-Lys*3-PyrS2-3Thi-34ClF-GlnR*3-NH2 |
| I-1583 | C3a-PL3-Asp-Ile-B5-Asp-3COOHF-Aib-Ala-Phe-Lys*3-PyrS2-3Thi-34ClF-GlnR*3-NH2 |
| I-1584 | Bua-PL3-Asp-Ile-B5-Asp-3COOHF-Aib-Ala-Phe-Lys*3-PyrS2-3Thi-34ClF-GlnR*3-NH2 |
| I-1585 | Bua-PL3-Asp-Ile-B5-Asp-3COOHF-Aib-Ala-Phe-Lys*3-PyrS2-3Thi-34ClF-GlnR*3-NH2 |
| I-1586 | Cpc-PL3-Asp-Ile-B5-Asp-3COOHF-Aib-Ala-Phe-Lys*3-PyrS2-3Thi-34ClF-GlnR*3-NH2 |
| I-1587 | Cpc-PL3-Asp-Ile-B5-Asp-3COOHF-Aib-Ala-Phe-Lys*3-PyrS2-3Thi-34ClF-GlnR*3-NH2 |
| I-1588 | Cbc-PL3-Asp-Ile-B5-Asp-3COOHF-Aib-Ala-Phe-Lys*3-PyrS2-3Thi-34ClF-GlnR*3-NH2 |
| I-1589 | Cbc-PL3-Asp-Ile-B5-Asp-3COOHF-Aib-Ala-Phe-Lys*3-PyrS2-3Thi-34ClF-GlnR*3-NH2 |
| I-1590 | CypCO-PL3-Asp-Ile-B5-Asp-3COOHF-Aib-Ala-Phe-Lys*3-PyrS2-3Thi-34ClF-GlnR*3-NH2 |
| I-1591 | CypCO-PL3-Asp-Ile-B5-Asp-3COOHF-Aib-Ala-Phe-Lys*3-PyrS2-3Thi-34ClF-GlnR*3-NH2 |
| I-1592 | 4THPCO-PL3-Asp-Ile-B5-Asp-3COOHF-Aib-Ala-Phe-Lys*3-PyrS2-3Thi-34ClF-GlnR*3-NH2 |
| I-1593 | 4THPCO-PL3-Asp-Ile-B5-Asp-3COOHF-Aib-Ala-Phe-Lys*3-PyrS2-3Thi-34ClF-GlnR*3-NH2 |
| I-1594 | Ac-PL3-Asp-Ile-B5-Asp-3COOHF-Aib-Ala-Phe-Lys*3-PyrS2-Phe-Phe-GlnR*3-NH2 |
| I-1595 | Ac-PL3-Asp-Ile-B5-Asp-3COOHF-Aib-Ala-Phe-Lys*3-PyrS2-Phe-Phe-GlnR*3-NH2 |
| I-1596 | Ac-PL3-Asp-Ile-B5-Asp-3COOHF-Aib-Ala-Phe-Lys*3-PyrS2-Phe-2ClF-GlnR*3-NH2 |
| I-1597 | Ac-PL3-Asp-Ile-B5-Asp-3COOHF-Aib-Ala-Phe-Lys*3-PyrS2-Phe-2ClF-GlnR*3-NH2 |
| I-1598 | Ac-PL3-Asp-Ile-B5-Asp-3COOHF-Aib-Ala-Phe-Lys*3-PyrS2-Phe-3ClF-GlnR*3-NH2 |
| I-1599 | Ac-PL3-Asp-Ile-B5-Asp-3COOHF-Aib-Ala-Phe-Lys*3-PyrS2-Phe-3ClF-GlnR*3-NH2 |
| I-1600 | Ac-PL3-Asp-Ile-B5-Asp-3COOHF-Aib-Ala-Phe-Lys*3-PyrS2-Phe-4ClF-GlnR*3-NH2 |
| I-1601 | Ac-PL3-Asp-Ile-B5-Asp-3COOHF-Aib-Ala-Phe-Lys*3-PyrS2-Phe-3MeF-GlnR*3-NH2 |
| I-1602 | Ac-PL3-Asp-Ile-B5-Asp-3COOHF-Aib-Ala-Phe-Lys*3-PyrS2-Phe-2BrF-GlnR*3-NH2 |
| I-1603 | Ac-PL3-Asp-Ile-B5-Asp-3COOHF-Aib-Ala-Phe-Lys*3-PyrS2-Phe-2BrF-GlnR*3-NH2 |
| I-1604 | Ac-PL3-Asp-Ile-B5-Asp-3COOHF-Aib-Ala-Phe-Lys*3-PyrS2-Phe-3BrF-GlnR*3-NH2 |
| I-1605 | Ac-PL3-Asp-Ile-B5-Asp-3COOHF-Aib-Ala-Phe-Lys*3-PyrS2-Phe-3BrF-GlnR*3-NH2 |
| I-1606 | Ac-PL3-Asp-Ile-B5-Asp-3COOHF-Aib-Ala-Phe-Lys*3-PyrS2-Phe-4BrF-GlnR*3-NH2 |
| I-1607 | Ac-PL3-Asp-Ile-B5-Asp-3COOHF-Aib-Ala-Phe-Lys*3-PyrS2-Phe-4BrF-GlnR*3-NH2 |
| I-1608 | Ac-PL3-Asp-Ile-B5-Asp-3COOHF-Aib-Ala-Phe-Lys*3-PyrS2-Phe-3F3MeF-GlnR*3-NH2 |
| I-1609 | Ac-PL3-Asp-Ile-B5-Asp-3COOHF-Aib-Ala-Phe-Lys*3-PyrS2-Phe-3F3MeF-GlnR*3-NH2 |
| I-1610 | Ac-PL3-Asp-Ile-B5-Asp-3COOHF-Aib-Ala-Phe-Lys*3-PyrS2-Phe-4F3MeF-GlnR*3-NH2 |
| I-1611 | Ac-PL3-Asp-Ile-B5-Asp-3COOHF-Aib-Ala-Phe-Lys*3-PyrS2-Phe-4F3MeF-GlnR*3-NH2 |
| I-1612 | Ac-PL3-Asp-Ile-B5-Asp-3COOHF-Aib-Ala-Phe-Lys*3-PyrS2-3Thi-2BrF-GlnR*3-NH2 |
| I-1613 | Ac-PL3-Asp-Ile-B5-Asp-3COOHF-Aib-Ala-Phe-Lys*3-PyrS2-3Thi-3BrF-GlnR*3-NH2 |
| I-1614 | Ac-PL3-Asp-Ile-B5-Asp-3COOHF-Aib-Ala-Phe-Lys*3-PyrS2-3Thi-3F3MeF-GlnR*3-NH2 |
| I-1615 | Ac-PL3-Asp-Ile-B5-Asp-3COOHF-Aib-Ala-Phe-Lys*3-PyrS2-3Thi-4F3MeF-GlnR*3-NH2 |
| I-1616 | Ac-S5-Ala-Asp-Cha-B5-Asp-3COOHF-Aib-Ala-Phe-Lys*3-PyrS2-Phe-34ClF-GlnR*3-NH2 |
| I-1617 | Ac-S5-Pro-Asp-Cha-B5-Asp-3COOHF-Aib-Ala-Phe-Lys*3-PyrS2-Phe-34ClF-GlnR*3-NH2 |
| I-1618 | Ac-S5-Val-Asp-Cha-B5-Asp-3COOHF-Aib-Ala-Phe-Lys*3-PyrS2-Phe-34ClF-GlnR*3-NH2 |
| I-1619 | Ac-S5-Glu-Asp-Cha-B5-Asp-3COOHF-Aib-Ala-Phe-Lys*3-PyrS2-Phe-34ClF-GlnR*3-NH2 |
| I-1620 | Ac-S5-Ala-Asp-Chg-B5-Asp-3COOHF-Aib-Ala-Phe-Lys*3-PyrS2-Phe-34ClF-GlnR*3-NH2 |
| I-1621 | Ac-S5-Pro-Asp-Chg-B5-Asp-3COOHF-Aib-Ala-Phe-Lys*3-PyrS2-Phe-34ClF-GlnR*3-NH2 |
| I-1622 | Ac-S5-Val-Asp-Chg-B5-Asp-3COOHF-Aib-Ala-Phe-Lys*3-PyrS2-Phe-34ClF-GlnR*3-NH2 |
| I-1623 | Ac-S5-Glu-Asp-Chg-B5-Asp-3COOHF-Aib-Ala-Phe-Lys*3-PyrS2-Phe-34ClF-GlnR*3-NH2 |
| I-1624 | Ac-S6-Ala-Asp-Cha-B5-Asp-3COOHF-Aib-Ala-Phe-Lys*3-PyrS2-Phe-34ClF-GlnR*3-NH2 |
| I-1625 | Ac-S6-Ala-Asp-Chg-B5-Asp-3COOHF-Aib-Ala-Phe-Lys*3-PyrS2-Phe-34ClF-GlnR*3-NH2 |
| I-1626 | Ac-S5-Ala-Asp-Cha-B5-Asp-3COOHF-Aib-Ala-Phe-Lys*3-PyrS1-Phe-34ClF-GlnR*3-NH2 |
| I-1627 | Ac-S5-Ala-Asp-Chg-B5-Asp-3COOHF-Aib-Ala-Phe-Lys*3-PyrS1-Phe-34ClF-GlnR*3-NH2 |
| I-1628 | Ac-PL3-Asp-Npg-B5-Asp-3COOHF-Aib-Ala-Phe-Lys*3-PyrS2-2F3MeF-BztA-GlnR*3-Lys-NH2 |
| I-1629 | Ac-PL3-Asp-Npg-B5-Asp-3COOHF-Aib-Ala-Phe-Lys*3-PyrS2-2F3MeF-BztA-GlnR*3-Lys-NH2 |
| I-1630 | Ac-PL3-Asn-DipA-B5-Asp-3COOHF-CyLeu-Ala-Phe-Lys*3-PyrS2-3Thi-34ClF-GlnR*3-NH2 |
| I-1631 | Ac-PL3-aThr-DipA-B5-Asp-3COOHF-CyLeu-Ala-Phe-Lys*3-PyrS2-3Thi-34ClF-GlnR*3-NH2 |
| I-1632 | Ac-PL3-Asp-DipA-B5-Asn-3COOHF-CyLeu-Ala-Phe-Lys*3-PyrS2-3Thi-34ClF-GlnR*3-NH2 |
| I-1633 | Ac-PL3-Asp-DipA-B5-aThr-3COOHF-CyLeu-Ala-Phe-Lys*3-PyrS2-3Thi-34ClF-GlnR*3-NH2 |
| I-1634 | Ac-PL3-Asp-DipA-B5-Asp-3COOHF-nLeu-Ala-Phe-Lys*3-PyrS2-3Thi-34ClF-GlnR*3-NH2 |
| I-1635 | Ac-PL3-Asp-DipA-B5-Asp-3COOHF-Thr-Ala-Phe-Lys*3-PyrS2-3Thi-34ClF-GlnR*3-NH2 |
| I-1636 | Ac-PL3-Asn-DipA-B5-Asp-3COOHF-nLeu-Ala-Phe-Lys*3-PyrS2-3Thi-34ClF-GlnR*3-NH2 |
| I-1637 | Ac-PL3-aThr-DipA-B5-Asp-3COOHF-nLeu-Ala-Phe-Lys*3-PyrS2-3Thi-34ClF-GlnR*3-NH2 |
| I-1638 | Ac-PL3-Asp-DipA-B5-Asn-3COOHF-nLeu-Ala-Phe-Lys*3-PyrS2-3Thi-34ClF-GlnR*3-NH2 |
| I-1639 | Ac-PL3-Asp-DipA-B5-aThr-3COOHF-nLeu-Ala-Phe-Lys*3-PyrS2-3Thi-34ClF-GlnR*3-NH2 |
| I-1640 | Ac-PL3-Asp-DipA-B5-Asp-3COOHF-CyLeu-Ala-Phe-Lys*3-PyrS2-2F3MeF-34ClF-GlnR*3-NH2 |
| I-1641 | Ac-PL3-Asp-Cha-B5-Asp-3COOHF-Aib-Ala-Phe-Lys*3-PyrS2-2ClF-BztA-GlnR*3-Leu-NH2 |
| I-1642 | Ac-PL3-Asp-Cha-B5-Asp-3COOHF-Aib-Ala-Phe-Lys*3-PyrS2-2ClF-BztA-GlnR*3-Leu-NH2 |
| I-1643 | Ac-PL3-Asp-Cha-B5-Asp-3COOHF-Aib-Ala-Phe-Lys*3-PyrS2-2ClF-BztA-GlnR*3-Ala-NH2 |
| I-1644 | Ac-PL3-Asp-Cha-B5-Asp-3COOHF-Aib-Ala-Phe-Lys*3-PyrS2-2ClF-BztA-GlnR*3-Ala-NH2 |
| I-1645 | Ac-PL3-Asp-Cha-B5-Asp-3COOHF-Aib-Ala-Phe-Lys*3-PyrS2-Phe-BztA-GlnR*3-Leu-NH2 |
| I-1646 | Ac-PL3-Asp-Cha-B5-Asp-3COOHF-Aib-Ala-Phe-Lys*3-PyrS2-Phe-BztA-GlnR*3-Leu-NH2 |
| I-1647 | Ac-PL3-Asp-Cha-B5-Asp-3COOHF-Aib-Ala-Phe-Lys*3-PyrS2-Phe-BztA-GlnR*3-Ala-NH2 |
| I-1648 | Ac-PL3-Asp-Cha-B5-Asp-3COOHF-Aib-Ala-Phe-Lys*3-PyrS2-Phe-BztA-GlnR*3-NH2 |
| I-1649 | Ac-PL3-Asp-Cha-B5-Asp-3COOHF-Aib-Ala-Phe-Lys*3-PyrS2-Phe-BztA-GlnR*3-NH2 |
| I-1650 | Ac-PL3-Asp-Cha-B5-Asp-3COOHF-Aib-Ala-Phe-Lys*3-PyrS2-2FF-BztA-GlnR*3-Leu-NH2 |
| I-1651 | Ac-PL3-Asp-Cha-B5-Asp-3COOHF-Aib-Ala-Phe-Lys*3-PyrS2-2FF-BztA-GlnR*3-Leu-NH2 |
| I-1652 | Ac-PL3-Asp-Cha-B5-Asp-3COOHF-Aib-Ala-Phe-Lys*3-PyrS2-2FF-BztA-GlnR*3-Ala-NH2 |
| I-1653 | Ac-PL3-Asp-Cha-B5-Asp-3COOHF-Aib-Ala-Phe-Lys*3-PyrS2-2FF-BztA-GlnR*3-NH2 |
| I-1654 | Ac-PL3-Asp-Cha-B5-Asp-3COOHF-Aib-Ala-Phe-Lys*3-PyrS2-2F3MeF-BztA-GlnR*3-Leu-NH2 |
| I-1655 | Ac-PL3-Asp-Cha-B5-Asp-3COOHF-Aib-Ala-Phe-Lys*3-PyrS2-2F3MeF-BztA-GlnR*3-Leu-NH2 |
| I-1656 | Ac-PL3-Asp-Cha-B5-Asp-3COOHF-Aib-Ala-Phe-Lys*3-PyrS2-2F3MeF-BztA-GlnR*3-Ala-NH2 |
| I-1657 | Ac-PL3-Asp-Cha-B5-Asp-3COOHF-Aib-Ala-Phe-Lys*3-PyrS2-2F3MeF-BztA-GlnR*3-Ala-NH2 |
| I-1658 | Ac-PL3-Asp-Cha-B5-Asp-3COOHF-Aib-Ala-Phe-Lys*3-PyrS2-2F3MeF-BztA-GlnR*3-NH2 |
| I-1659 | Ac-PL3-Asp-Cha-B5-Asp-3COOHF-Aib-Ala-Phe-Lys*3-PyrS2-2F3MeF-BztA-GlnR*3-NH2 |
| I-1660 | Ac-PL3-Asp-Npg-B5-Asp-3COOHF-Aib-Ala-Phe-Lys*3-PyrS2-[Bnc]2NH2F-BztA-GlnR*3-Ala- |
| | NH2 |
| I-1661 | Ac-PL3-Asp-Npg-B5-Asp-3COOHF-Aib-Ala-Phe-Lys*3-PyrS2-[Bnc]2NH2F-BztA-GlnR*3-Ala- |
| | NH2 |
| I-1662 | Ac-PL3-Asp-Npg-B5-Asp-3COOHF-Aib-Ala-Phe-Lys*3-PyrS2-[Phc]2NH2F-BztA-GlnR*3-Ala- |
| | NH2 |
| I-1663 | Ac-PL3-Asp-Npg-B5-Asp-3COOHF-Aib-Ala-Phe-Lys*3-PyrS2-[Phc]2NH2F-BztA-GlnR*3-Ala- |
| | NH2 |
| I-1664 | Ac-PL3-Asp-Npg-B5-Asp-3COOHF-Aib-Ala-Phe-Lys*3-PyrS2-[BiPh]2NH2F-BztA-GlnR*3-Ala- |
| | NH2 |
| I-1665 | Ac-PL3-Asp-Npg-B5-Asp-3COOHF-Aib-Ala-Phe-Lys*3-PyrS2-[BiPh]2NH2F-BztA-GlnR*3-Ala- |
| | NH2 |
| I-1666 | Ac-PL3-Asp-Npg-B5-Asp-3COOHF-Aib-Ala-Phe-Lys*3-PyrS2-[MePipAc]2NH2F-BztA-GlnR*3- |
| | Ala-NH2 |
| I-1667 | Ac-PL3-Asp-Npg-B5-Asp-3COOHF-Aib-Ala-Phe-Lys+3-PyrS2-[2IAPAc]2NH2F-BztA-GlnR*3- |
| | Ala-NH2 |
| I-1668 | Ac-PL3-Asp-Npg-B5-Asp-3COOHF-Aib-Ala-Phe-Lys*3-PyrS2-[2IAPAc]2NH2F-BztA-GlnR*3- |
| | Ala-NH2 |
| I-1669 | Ac-PL3-Asp-Npg-B5-Asp-3COOHF-Aib-Ala-Phe-Lys*3-PyrS2-[2IAPAc]2NH2F-BztA-GlnR*3- |
| | Ala-NH2 |
| I-1670 | Ac-PL3-Asp-Npg-B5-Asp-3COOHF-Aib-Ala-Phe-Lys+3-PyrS2-[3PyAc]2NH2F-BztA-GlnR*3- |
| | Ala-NH2 |
| I-1671 | Ac-PL3-Asp-Npg-B5-Asp-3COOHF-Aib-Ala-Phe-Lys*3-PyrS2-[3PyAc]2NH2F-BztA-GlnR*3- |
| | Ala-NH2 |
| I-1672 | Ac-PL3-Asp-Npg-B5-Asp-3COOHF-Aib-Ala-Phe-Lys*3-PyrS2-[2PyCypCO]2NH2F-BztA- |
| | GlnR*3-Ala-NH2 |
| I-1673 | Ac-S5-Asp-Asp-Npg-B5-Asp-3COOHF-Aib-Ala-Phe-Lys*3-PyrS2-Phe-34ClF-GlnR*3-NH2 |
| I-1674 | Ac-S5-Asp-Asn-Npg-B5-Asp-3COOHF-Aib-Ala-Phe-Lys*3-PyrS2-Phe-34ClF-GlnR*3-NH2 |
| I-1675 | Ac-S5-Asp-Ser-Npg-B5-Asp-3COOHF-Aib-Ala-Phe-Lys*3-PyrS2-Phe-34ClF-GlnR*3-NH2 |
| I-1676 | Ac-S5-Asp-Asp-Npg-B5-Glu-3COOHF-Aib-Ala-Phe-Lys*3-PyrS2-Phe-34ClF-GlnR*3-NH2 |
| I-1677 | Ac-S5-Glu-Asn-Npg-B5-Asp-3COOHF-Aib-Ala-Phe-Lys*3-PyrS2-Phe-34ClF-GlnR*3-NH2 |
| I-1678 | Ac-S5-Glu-Ser-Npg-B5-Asp-3COOHF-Aib-Ala-Phe-Lys*3-PyrS2-Phe-34ClF-GlnR*3-NH2 |
| I-1679 | Ac-S5-Glu-Asp-Npg-B5-Glu-3COOHF-Aib-Ala-Phe-Lys*3-PyrS2-Phe-34ClF-GlnR*3-NH2 |
| I-1680 | Ac-PL3-Asn-DipA-B5-Asp-3COOHF-nLeu-Ala-Phe-Lys*3-PyrS2-3Thi-BztA-GlnR*3-NH2 |
| I-1681 | Ac-PL3-Ser-DipA-B5-Asp-3COOHF-nLeu-Ala-Phe-Lys*3-PyrS2-3Thi-BztA-GlnR*3-NH2 |
| I-1682 | Ac-PL3-Thr-DipA-B5-Asp-3COOHF-nLeu-Ala-Phe-Lys*3-PyrS2-3Thi-BztA-GlnR*3-NH2 |
| I-1683 | Ac-PL3-Asp-DipA-B5-Ser-3COOHF-nLeu-Ala-Phe-Lys*3-PyrS2-3Thi-BztA-GlnR*3-NH2 |
| I-1684 | Ac-PL3-Asp-DipA-B5-Thr-3COOHF-nLeu-Ala-Phe-Lys*3-PyrS2-3Thi-BztA-GlnR*3-NH2 |
| I-1685 | Ac-PL3-Asp-DipA-B5-aThr-3COOHF-nLeu-Ala-Phe-Lys*3-PyrS2-3Thi-BztA-GlnR*3-NH2 |
| I-1686 | Ac-PL3-Asp-DipA-B5-Hse-3COOHF-nLeu-Ala-Phe-Lys*3-PyrS2-3Thi-BztA-GlnR*3-NH2 |
| I-1687 | Ac-PL3-aThr-DipA-B5-Asp-3COOHF-nLeu-Ala-3Thi-Lys*3-PyrS2-3Thi-BztA-GlnR*3-NH2 |
| I-1688 | Ac-S5-Ala-Asp-DipA-B5-Asp-3COOHF-nLeu-Ala-Phe-Lys*3-PyrS2-Phe-34ClF-GlnR*3-NH2 |
| I-1689 | Ac-S5-Ala-Asp-DipA-B5-Asp-3COOHF-nLeu-Ala-Phe-Lys*3-PyrS1-Phe-34ClF-GlnR*3-NH2 |
| I-1690 | Ac-S6-Ala-Asp-DipA-B5-Asp-3COOHF-nLeu-Ala-Phe-Lys*3-PyrS2-Phe-34ClF-GlnR*3-NH2 |
| I-1691 | Ac-S6-Ala-Asp-DipA-B5-Asp-3COOHF-nLeu-Ala-Phe-Lys*3-PyrS1-Phe-34ClF-GlnR*3-NH2 |
| I-1692 | Ac-S5-Ala-Asp-DipA-B5-Asp-3COOHF-Aib-Ala-Phe-Lys*3-PyrS2-Phe-34ClF-GlnR*3-NH2 |
| I-1693 | Ac-S5-Ala-Asp-DipA-B5-Asp-3COOHF-Aib-Ala-Phe-Lys*3-PyrS1-Phe-34ClF-GlnR*3-NH2 |
| I-1694 | Ac-S5-Pro-Asp-DipA-B5-Asp-3COOHF-Aib-Ala-Phe-Lys*3-PyrS1-Phe-34ClF-GlnR*3-NH2 |
| I-1695 | Ac-S6-Pro-Asp-DipA-B5-Asp-3COOHF-Aib-Ala-Phe-Lys*3-PyrS2-Phe-34ClF-GlnR*3-NH2 |
| I-1696 | Ac-S6-Pro-Asp-DipA-B5-Asp-3COOHF-Aib-Ala-Phe-Lys*3-PyrS1-Phe-34ClF-GlnR*3-NH2 |
| I-1697 | Ac-PL3-Asp-Ile-B5-Asp-3COOHF-Ala-Ala-Phe-Lys*3-PyrS2-Phe-34ClF-GlnR*3-NH2 |
| I-1698 | Ac-PL3-Asp-Ile-B5-Asp-3COOHF-Ala-Ala-Phe-Lys*3-PyrS1-Phe-34ClF-GlnR*3-NH2 |
| I-1699 | Ac-PL3-Asp-Leu-B5-Asp-3COOHF-Ala-Ala-Phe-Lys*3-PyrS2-Phe-34ClF-GlnR*3-NH2 |
| I-1700 | Ac-PL3-Asp-Leu-B5-Asp-3COOHF-Ala-Ala-Phe-Lys*3-PyrS1-Phe-34ClF-GlnR*3-NH2 |
| I-1701 | Ac-PL3-Asp-DipA-B5-Asp-3COOHF-Ala-Ala-Phe-Lys*3-PyrS2-3Thi-34ClF-GlnR*3-[mPEG8]- |
| | Lys-NH2 |
| I-1702 | Ac-PL3-Asp-DipA-B5-Asp-3COOHF-Ala-Ala-Phe-Lys*3-PyrS2-3Thi-34ClF-GlnR*3-[mPEG37]- |
| | Lys-NH2 |
| I-1703 | Ac-PL3-Asp-DipA-B5-Asp-3COOHF-Ala-Ala-Phe-Lys*3-PyrS2-3Thi-34ClF-GlnR*3-Ala-Ala- |
| | [mPEG8]-Lys-NH2 |
| I-1704 | Ac-PL3-Asp-DipA-B5-Asp-3COOHF-Ala-Ala-Phe-Lys*3-PyrS2-3Thi-34ClF-GlnR*3-Ala-Ala- |
| | [mPEG37]-Lys-NH2 |
| I-1705 | Ac-PL3-Asp-DipA-B5-Asp-3COOHF-Ala-Ala-Phe-Lys*3-PyrS2-3Thi-34ClF-GlnR*3-Ala-Ala- |
| | Ala-[mPEG8]-Lys |
| I-1706 | Ac-PL3-Asp-DipA-B5-Asp-3COOHF-Ala-Ala-Phe-Lys*3-PyrS2-3Thi-34ClF-GlnR*3-Ala-Ala- |
| | Ala-[mPEG37]-Lys |
| I-1707 | Ac-PL3-Asp-Npg-B5-Asp-3COOHF-Aib-Ala-Phe-Lys*3-PyrS2-3Thi-34ClF-GlnR*3-Ala-Ala-Ala- |
| | [mPEG8]-Lys |
| I-1708 | Ac-PL3-Asp-Npg-B5-Asp-3COOHF-Aib-Ala-Phe-Lys*3-PyrS2-3Thi-34ClF-GlnR*3-Ala-Ala-Ala- |
| | [mPEG37]-Lys |
| I-1709 | Ac-PL3-Asp-Npg-B5-Asp-3COOHF-Aib-Ala-Phe-Lys*3-PyrS2-3Thi-BztA-GlnR*3-OH |
| I-1710 | Bua-PL3-Asp-DipA-B5-Asp-3COOHF-CyLeu-Ala-Phe-Lys*3-PyrS2-3Thi-34ClF-GlnR*3-NH2 |
| I-1711 | Isovaleryl-PL3-Asp-DipA-B5-Asp-3COOHF-CyLeu-Ala-Phe-Lys*3-PyrS2-3Thi-34ClF-GlnR*3- |
| | NH2 |
| I-1712 | Cpc-PL3-Asp-DipA-B5-Asp-3COOHF-CyLeu-Ala-Phe-Lys*3-PyrS2-3Thi-34ClF-GlnR*3-NH2 |
| I-1713 | Cbc-PL3-Asp-DipA-B5-Asp-3COOHF-CyLeu-Ala-Phe-Lys*3-PyrS2-3Thi-34ClF-GlnR*3-NH2 |
| I-1714 | CypCO-PL3-Asp-DipA-B5-Asp-3COOHF-CyLeu-Ala-Phe-Lys*3-PyrS2-3Thi-34ClF-GlnR*3-NH2 |
| I-1715 | Bnc-PL3-Asp-DipA-B5-Asp-3COOHF-CyLeu-Ala-Phe-Lys*3-PyrS2-3Thi-34ClF-GlnR*3-NH2 |
| I-1716 | CF3CO-PL3-Asp-DipA-B5-Asp-3COOHF-CyLeu-Ala-Phe-Lys*3-PyrS2-3Thi-34ClF-GlnR*3-NH2 |
| I-1717 | 6QuiAc-PL3-Asp-DipA-B5-Asp-3COOHF-CyLeu-Ala-Phe-Lys*3-PyrS2-3Thi-34ClF-GlnR*3-NH2 |
| I-1718 | 124TriAc-PL3-Asp-DipA-B5-Asp-3COOHF-CyLeu-Ala-Phe-Lys*3-PyrS2-3Thi-34ClF-GlnR*3- |
| | NH2 |
| I-1719 | 5PymAc-PL3-Asp-DipA-B5-Asp-3COOHF-CyLeu-Ala-Phe-Lys*3-PyrS2-3Thi-34ClF-GlnR*3- |
| | NH2 |
| I-1720 | 2PyCypCO-PL3-Asp-DipA-B5-Asp-3COOHF-CyLeu-Ala-Phe-Lys*3-PyrS2-3Thi-34ClF-GlnR*3- |
| | NH2 |
| I-1721 | 2PyBu-PL3-Asp-DipA-B5-Asp-3COOHF-CyLeu-Ala-Phe-Lys*3-PyrS2-3Thi-34ClF-GlnR*3-NH2 |
| I-1722 | 2PyzCO-PL3-Asp-DipA-B5-Asp-3COOHF-CyLeu-Ala-Phe-Lys*3-PyrS2-3Thi-34ClF-GlnR*3- |
| | NH2 |
| I-1723 | Bua-PL3-Asn-DipA-B5-Asp-3COOHF-CyLeu-Ala-Phe-Lys*3-PyrS2-3Thi-34ClF-GlnR*3-NH2 |
| I-1724 | Isovalery1-PL3-Asn-DipA-B5-Asp-3COOHF-CyLeu-Ala-Phe-Lys*3-PyrS2-3Thi-34ClF-GlnR*3- |
| | NH2 |
| I-1725 | Cpc-PL3-Asn-DipA-B5-Asp-3COOHF-CyLeu-Ala-Phe-Lys*3-PyrS2-3Thi-34ClF-GlnR*3-NH2 |
| I-1726 | Cbc-PL3-Asn-DipA-B5-Asp-3COOHF-CyLeu-Ala-Phe-Lys*3-PyrS2-3Thi-34ClF-GlnR*3-NH2 |
| I-1727 | CypCO-PL3-Asn-DipA-B5-Asp-3COOHF-CyLeu-Ala-Phe-Lys*3-PyrS2-3Thi-34ClF-GlnR*3-NH2 |
| I-1728 | Bnc-PL3-Asn-DipA-B5-Asp-3COOHF-CyLeu-Ala-Phe-Lys*3-PyrS2-3Thi-34ClF-GlnR*3-NH2 |
| I-1729 | CF3CO-PL3-Asn-DipA-B5-Asp-3COOHF-CyLeu-Ala-Phe-Lys*3-PyrS2-3Thi-34ClF-GlnR*3-NH2 |
| I-1730 | 6QuiAc-PL3-Asn-DipA-B5-Asp-3COOHF-CyLeu-Ala-Phe-Lys*3-PyrS2-3Thi-34ClF-GlnR*3-NH2 |
| I-1731 | 124TriAc-PL3-Asn-DipA-B5-Asp-3COOHF-CyLeu-Ala-Phe-Lys*3-PyrS2-3Thi-34ClF-GlnR*3- |
| | NH2 |
| I-1732 | 2PyCypCO-PL3-Asn-DipA-B5-Asp-3COOHF-CyLeu-Ala-Phe-Lys*3-PyrS2-3Thi-34ClF-GlnR*3- |
| | NH2 |
| I-1733 | 2PyBu-PL3-Asn-DipA-B5-Asp-3COOHF-CyLeu-Ala-Phe-Lys*3-PyrS2-3Thi-34ClF-GlnR*3-NH2 |
| I-1734 | 2PyzCO-PL3-Asn-DipA-B5-Asp-3COOHF-CyLeu-Ala-Phe-Lys*3-PyrS2-3Thi-34ClF-GlnR*3- |
| | NH2 |
| I-1735 | Ac-PL3-Asp-Npg-B5-Asp-3COOHF-Aib-Ala-Phe-Lys*3-PyrS2-2NH2F-BztA-GlnR*3-Ala-NH2 |
| I-1736 | Ac-PL3-Asp-Npg-B5-Asp-3COOHF-Aib-Ala-Phe-Lys*3-PyrS2-2NH2F-BztA-GlnR*3-Ala-NH2 |
| I-1737 | Ac-PL3-Asp-Npg-B5-Asp-3COOHF-Aib-Ala-Phe-Lys*3-PyrS2-[124TriAc]2NH2F-BztA-GlnR*3- |
| | Ala-NH2 |
| I-1738 | Ac-PL3-Asp-Npg-B5-Asp-3COOHF-Aib-Ala-Phe-Lys*3-PyrS2-[124TriPr]2NH2F-BztA-GlnR*3- |
| | Ala-NH2 |
| I-1739 | Ac-PL3-Asp-Npg-B5-Asp-3COOHF-Aib-Ala-Phe-Lys*3-PyrS2-[6QuiAc]2NH2F-BztA-GlnR*3- |
| | Ala-NH2 |
| I-1740 | Ac-PL3-Asp-Npg-B5-Asp-3COOHF-Aib-Ala-Phe-Lys*3-PyrS2-[2PyAc]2NH2F-BztA-GlnR*3- |
| | Ala-NH2 |
| I-1741 | Ac-PL3-Asp-Npg-B5-Asp-3COOHF-Aib-Ala-Phe-Lys*3-PyrS2-[2PyPrpc]2NH2F-BztA-GlnR*3- |
| | Ala-NH2 |
| I-1742 | Ac-PL3-Asp-Npg-B5-Asp-3COOHF-Aib-Ala-Phe-Lys*3-PyrS2-[3PyPrpc]2NH2F-BztA-GlnR*3- |
| | Ala-NH2 |
| I-1743 | Ac-PL3-Asp-Npg-B5-Asp-3COOHF-Aib-Ala-Phe-Lys*3-PyrS2-[4PyPrpc]2NH2F-BztA-GlnR*3- |
| | Ala-NH2 |
| I-1744 | Ac-PL3-Asp-Npg-B5-Asp-3COOHF-Aib-Ala-Phe-Lys*3-PyrS2-[MeOPr]2NH2F-BztA-GlnR*3- |
| | Ala-NH2 |
| I-1745 | Ac-PL3-Asp-Npg-B5-Asp-3COOHF-Aib-Ala-Phe-Lys*3-PyrS2-[PhOPr]2NH2F-BztA-GlnR*3- |
| | Ala-NH2 |
| I-1746 | Ac-PL3-Asp-Npg-B5-Asp-3COOHF-Aib-Ala-Phe-Lys*3-PyrS2-[Me2MeOPr]2NH2F-BztA- |
| | GlnR*3-Ala-NH2 |
| I-1747 | Ac-PL3-Asp-Npg-B5-Asp-3COOHF-Aib-Ala-Phe-Lys*3-PyrS2-[Me2NAc]2NH2F-BztA-GlnR*3- |
| | Ala-NH2 |
| I-1748 | Ac-PL3-Asp-Npg-B5-Asp-3COOHF-Aib-Ala-Phe-Lys*3-PyrS2-[Me2NAc]2NH2F-BztA-GlnR*3- |
| | Ala-NH2 |
| I-1749 | Ac-PL3-Asp-Npg-B5-Asp-3COOHF-Aib-Ala-Phe-Lys*3-PyrS2-[Me2NPr]2NH2F-BztA-GlnR*3- |
| | Ala-NH2 |
| I-1750 | Ac-PL3-Asp-Npg-B5-Asp-3COOHF-Aib-Ala-Phe-Lys*3-PyrS2-[NdiMeButC]2NH2F-BztA- |
| | GlnR*3-Ala-NH2 |
| I-1751 | Ac-PL3-Asp-Npg-B5-Asp-3COOHF-Aib-Ala-Phe-Lys*3-PyrS2-[3IAPAc]2NH2F-BztA-GlnR*3- |
| | Ala-NH2 |
| I-1752 | Ac-PL3-Asp-Npg-B5-Asp-3COOHF-Aib-Ala-Phe-Lys*3-PyrS2-[15PyraPy]2NH2F-BztA-GlnR*3- |
| | Ala-NH2 |
| I-1753 | Ac-PL3-Asp-Npg-B5-Asp-3COOHF-Aib-Ala-Phe-Lys*3-PyrS2-[MorphAc]2NH2F-BztA-GlnR*3- |
| | Ala-NH2 |
| I-1754 | Ac-PL3-Asp-Npg-B5-Asp-3COOHF-Aib-Ala-Phe-Lys*3-PyrS2-[Nic]2NH2F-BztA-GlnR*3-Ala- |
| | NH2 |
| I-1755 | Ac-PL3-Asp-Npg-B5-Asp-3COOHF-Aib-Ala-Phe-Lys*3-PyrS2-[2PyzCO]2NH2F-BztA-GlnR*3- |
| | Ala-NH2 |
| I-1756 | Ac-PL3-Asp-Npg-B5-Asp-3COOHF-Aib-Ala-Phe-Lys*3-PyrS2-[5pymCO]2NH2F-BztA-GlnR*3- |
| | Ala-NH2 |
| I-1757 | Ac-PL3-Asp-Ile-B5-Asp-3COOHF-Aib-Ala-Phe-Lys*3-PyrS2-Phe-34ClF-dGlnR*3-NH2 |
| I-1758 | Ac-PL3-Asp-Ile-B5-Asp-3COOHF-Aib-Ala-Phe-Lys*3-PyrS2-Phe-34ClF-dGlnR*3-NHMe |
| I-1759 | Ac-PL3-Asp-Ile-B5-Asp-3COOHF-Aib-Ala-Phe-Lys*3-PyrS2-Phe-34ClF-dGlnR*3-Ala-NH2 |
| I-1760 | Ac-PL3-Asp-Ile-B5-Asp-3COOHF-Aib-Ala-Phe-1MeK*3-PyrS2-Phe-34ClF-dGlnR*3-NH2 |
| I-1761 | Ac-PL3-Asp-Ile-B5-Asp-3COOHF-Aib-Ala-Phe-1MeK*3-PyrS2-Phe-34ClF-dGlnR*3-NHMe |
| I-1762 | Ac-PL3-Asp-Ile-B5-Asp-3COOHF-Aib-Ala-Phe-1MeK*3-PyrS2-Phe-34ClF-dGlnR*3-Ala-NH2 |
| I-1763 | Ac-PL3-Asp-Npg-B5-Asp-3COOHF-Aib-Ala-Phe-Lys*3-PyrS2-[3FPyr2c]2NH2F-BztA-GlnR*3- |
| | Ala-NH2 |
| I-1764 | Ac-PL3-Asp-Npg-B5-Asp-3COOHF-Aib-Ala-Phe-Lys*3-PyrS2-[4FPyr3c]2NH2F-BztA-GlnR*3- |
| | Ala-NH2 |
| I-1765 | Ac-PL3-Asp-Npg-B5-Asp-3COOHF-Aib-Ala-Phe-Lys*3-PyrS2-[4FPyr3c]2NH2F-BztA-GlnR*3- |
| | Ala-NH2 |
| I-1766 | Ac-PL3-Asp-DipA-B5-Asp-3COOHF-CyLeu-Ala-Phe-Lys*3-PyrS2-2F3MeF-BztA-GlnR*3-NH2 |
| I-1767 | Ac-PL3-Asp-DipA-B5-Asp-3COOHF-CyLeu-Ala-Phe-Lys*3-PyrS2-2F3MeF-34ClF-GlnR*3-Pro- |
| | NH2 |
| I-1768 | Ac-PL3-Asp-DipA-B5-Asp-3COOHF-CyLeu-Ala-Phe-Lys*3-PyrS2-2F3MeF-BztA-GlnR*3-Pro- |
| | NH2 |
| I-1769 | Ac-PL3-Asp-DipA-B5-Asp-3COOHF-CyLeu-Ala-Phe-Lys*3-PyrS2-2F3MeF-34ClF-GlnR*3-Leu- |
| | NH2 |
| I-1770 | Ac-PL3-Asp-DipA-B5-Asp-3COOHF-CyLeu-Ala-Phe-Lys*3-PyrS2-2F3MeF-BztA-GlnR*3-Leu- |
| | NH2 |
| I-1771 | Ac-PL3-Asp-DipA-B5-Asp-3COOHF-CyLeu-Ala-Phe-Lys*3-PyrS2-2F3MeF-34ClF-GlnR*3-Ser- |
| | NH2 |
| I-1772 | Ac-PL3-Asp-DipA-B5-Asp-3COOHF-CyLeu-Ala-Phe-Lys*3-PyrS2-2F3MeF-BztA-GlnR*3-Ser- |
| | NH2 |
| I-1773 | Ac-PL3-Asp-DipA-B5-Asp-3COOHF-CyLeu-Ala-Phe-Lys*3-PyrS2-2F3MeF-34ClF-GlnR*3-Val- |
| | NH2 |
| I-1774 | Ac-PL3-Asp-DipA-B5-Asp-3COOHF-CyLeu-Ala-Phe-Lys*3-PyrS2-2F3MeF-BztA-GlnR*3-Val- |
| | NH2 |
| I-1775 | Ac-PL3-Asp-DipA-B5-Asp-3COOHF-CyLeu-Ala-Phe-Lys*3-PyrS2-2F3MeF-BztA-GlnR*3-Val- |
| | NH2 |
| I-1776 | Ac-PL3-Asp-DipA-B5-Asp-3COOHF-CyLeu-Ala-Phe-Lys*3-PyrS2-2F3MeF-34ClF-GlnR*3-Phe- |
| | NH2 |
| I-1777 | Ac-PL3-Asp-DipA-B5-Asp-3COOHF-CyLeu-Ala-Phe-Lys*3-PyrS2-2F3MeF-BztA-GlnR*3-Phe- |
| | NH2 |
| I-1778 | Ac-PL3-Asp-Ile-B5-Asp-3COOHF-Aib-Ala-Phe-GlnR*3-PyrS2-Phe-34ClF-dLys*3-Ala-NH2 |
| I-1779 | Ac-PL3-Asp-Ile-B5-Asp-3COOHF-Aib-Ala-Phe-Orn*3-PyrS2-Phe-34ClF-dGlnR*3-NH2 |
| I-1780 | Ac-PL3-Asp-Ile-B5-Asp-3COOHF-Aib-Ala-Phe-Orn*3-PyrS2-Phe-34ClF-dGlnR*3-NH2 |
| I-1781 | Ac-PL3-Asp-Ile-B5-Asp-3COOHF-Aib-Ala-Phe-Orn*3-PyrS2-Phe-34ClF-dGlnR*3-NHMe |
| I-1782 | Ac-PL3-Asp-Ile-B5-Asp-3COOHF-Aib-Ala-Phe-Orn*3-PyrS2-Phe-34ClF-dGlnR*3-Ala-NH2 |
| I-1783 | Ac-S6-Val-Asp-DipA-B5-Asp-3COOHF-Aib-Ala-Phe-Lys*3-PyrS1-Phe-34ClF-GlnR*3-NH2 |
| I-1784 | Ac-S5-Glu-Asp-DipA-B5-Asp-3COOHF-Aib-Ala-Phe-Lys*3-PyrS1-Phe-34ClF-GlnR*3-NH2 |
| I-1785 | Ac-S5-Leu-Asp-DipA-B5-Asp-3COOHF-Aib-Ala-Phe-Lys*3-PyrS1-Phe-34ClF-GlnR*3-NH2 |
| I-1786 | Ac-PL3-Asp-Ile-B5-Asp-Me2Gln-Aib-Ala-Phe-Lys*3-PyrS2-Phe-34ClF-GlnR*3-Ala-NH2 |
| I-1787 | Ac-PL3-Asp-DipA-B5-Asp-3COOHF-Ala-Ala-Phe-Lys*3-PyrS2-3Thi-34ClF-GlnR*3-Ala-Ala- |
| | [Ac-dPEG2]-Lys-NH2-NH2 |
| I-1788 | Ac-PL3-Asp-DipA-B5-Asp-3COOHF-Ala-Ala-Phe-Lys*3-PyrS2-3Thi-34ClF-GlnR*3-Ala-Ala- |
| | [Ac-PEG8]-Lys-NH2-NH2 |
| I-1789 | Ac-PL3-Asp-DipA-B5-Asp-3COOHF-Ala-Ala-Phe-Lys*3-PyrS2-3Thi-34ClF-GlnR*3-Ala-Ala- |
| | [Oct-dPEG2]-Lys-NH2-NH2 |
| I-1790 | Ac-PL3-Asp-DipA-B5-Asp-3COOHF-Ala-Ala-Phe-Lys*3-PyrS2-3Thi-34ClF-GlnR*3-Ala-Ala- |
| | [Oct-PEG8]-Lys-NH2-NH2 |
| I-1791 | Ac-PL3-Asp-DipA-B5-Asp-3COOHF-Ala-Ala-Phe-Lys*3-PyrS2-3Thi-34ClF-GlnR*3-Ala-Ala- |
| | [C18-dPEG2]-Lys-NH2-NH2 |
| I-1792 | Ac-PL3-Asp-DipA-B5-Asp-3COOHF-Ala-Ala-Phe-Lys*3-PyrS2-3Thi-34ClF-GlnR*3-Ala-Ala- |
| | [C18-PEG8]-Lys-NH2-NH2 |
| I-1793 | Ac-PL3-Asp-Ile-B5-Asp-3COOHF-GlnPDA*3-Ala-Phe-Leu-PyrS2-Phe-34ClF-GlnR*3-NH2 |
| I-1794 | Ac-PL3-Asp-Ile-B5-Asp-3COOHF-GlnPDA*3-Ala-Phe-Ala-PyrS2-Phe-34ClF-GlnR*3-NH2 |
| I-1795 | Ac-PL3-Asp-Ile-B5-Asp-3COOHF-GlnPDA*3-Ala-Phe-Val-PyrS2-Phe-34ClF-GlnR*3-NH2 |
| I-1796 | Ac-PL3-Asp-Ile-B5-Asp-3COOHF-GlnBDA*3-Ala-Phe-Leu-PyrS2-Phe-34ClF-GlnR*3-NH2 |
| I-1797 | Ac-PL3-Asp-Ile-B5-Asp-3COOHF-GlnBDA*3-Ala-Phe-Ala-PyrS2-Phe-34ClF-GlnR*3-NH2 |
| I-1798 | Ac-PL3-Asp-Ile-B5-Asp-3COOHF-GlnBDA*3-Ala-Phe-Val-PyrS2-Phe-34ClF-GlnR*3-NH2 |
| I-1799 | Ac-PL3-Asp-Ile-B5-Asp-3COOHF-GlnMePDA*3-Ala-Phe-Leu-PyrS2-Phe-34ClF-GlnR*3-NH2 |
| I-1800 | Ac-PL3-Asp-Ile-B5-Asp-3COOHF-GlnMePDA*3-Ala-Phe-Ala-PyrS2-Phe-34ClF-GlnR*3-NH2 |
| I-1801 | Ac-PL3-Asp-Ile-B5-Asp-3COOHF-GlnMePDA*3-Ala-Phe-Val-PyrS2-Phe-34ClF-GlnR*3-NH2 |
| I-1802 | Ac-PL3-Asp-Ile-B5-Asp-3COOHF-GlnR*3-Ala-Phe-Leu-PyrS2-Phe-34ClF-GlnMePDA*3-NH2 |
| I-1803 | Ac-PL3-Asp-Ile-B5-Asp-3COOHF-GlnR*3-Ala-Phe-Ala-PyrS2-Phe-34ClF-GlnMePDA*3-NH2 |
| I-1804 | Ac-PL3-Asp-Ile-B5-Asp-3COOHF-GlnR*3-Ala-Phe-Val-PyrS2-Phe-34ClF-GlnMePDA*3-NH2 |
| I-1805 | Ac-S5-TfeGA-Asp-DipA-B5-Asp-3COOHF-Aib-Ala-Phe-Lys*3-PyrS1-Phe-34ClF-GlnR*3-NH2 |
| I-1806 | Ac-S5-3COOHF-Asp-DipA-B5-Asp-3COOHF-Aib-Ala-Phe-Lys*3-PyrS1-Phe-34ClF-GlnR*3-NH2 |
| I-1807 | Ac-S5-Thr-Asp-DipA-B5-Asp-3COOHF-Aib-Ala-Phe-Lys*3-PyrS1-Phe-34ClF-GlnR*3-NH2 |
| I-1808 | Ac-S5-Phe-Asp-DipA-B5-Asp-3COOHF-Aib-Ala-Phe-Lys*3-PyrS1-Phe-34ClF-GlnR*3-NH2 |
| I-1809 | Ac-PL3-Asp-Npg-B5-Asp-3COOHF-Aib-Ala-Phe-TriAzLys*3-S3MePyrSc7-3Thi-BztA-sAla*3- |
| | Ala-NH2 |
| I-1810 | Ac-PL3-Asp-Npg-B5-Asp-3COOHF-Aib-Ala-Phe-TriAzLys*3-R3MePyrSc7-3Thi-BztA-sAla*3- |
| | Ala-NH2 |
| I-1811 | Ac-PL3-Asp-Ile-B5-Asp-3COOHF-GlnR*3-Ala-Phe-Ala-PyrS2-Phe-34ClF-GlnT4CyMe*3-NH2 |
| I-1812 | Ac-PL3-Asp-Npg-B5-Asp-3COOHF-Aib-Ala-Phe-TriAzLys*3-S3iPrPyrSc7-3Thi-BztA-sAla*3- |
| | Ala-NH2 |
| I-1813 | Ac-PL3-Asp-Npg-B5-Asp-3COOHF-Aib-Ala-Phe-TriAzLys*3-S3iPrPyrSc7-3Thi-BztA-sAla*3- |
| | Ala-NH2 |
| I-1814 | Ac-PL3-Asp-Npg-B5-Asp-3COOHF-Aib-Ala-Phe-TriAzLys*3-R3iPrPyrSc7-3Thi-BztA-sAla*3- |
| | Ala-NH2 |
| I-1815 | Ac-PL3-Asp-Npg-B5-Asp-3COOHF-Aib-Ala-Phe-Lys*3-PyrS2-3Thi-BztA-GlnR*3-Thr-Ala- |
| | [mPEG8]-Lys-NH2 |
| I-1816 | Ac-PL3-Asp-Npg-B5-Asp-3COOHF-Aib-Ala-Phe-Lys*3-PyrS2-3Thi-BztA-GlnR*3-Thr-Ala- |
| | [mPEG8]-Lys-NH2 |
| I-1817 | Ac-PL3-Asp-Npg-B5-Asp-3COOHF-Aib-Ala-Phe-Lys*3-PyrS2-3Thi-BztA-GlnR*3-Val-Glu- |
| | [mPEG8]-Lys-NH2 |
| I-1818 | Ac-PL3-Asp-Npg-B5-Asp-3COOHF-Aib-Ala-Phe-Lys*3-PyrS2-3Thi-BztA-GlnR*3-Val-Thr- |
| | [mPEG8]-Lys-NH2 |
| I-1819 | Ac-PL3-Asp-Npg-B5-Asp-3COOHF-Aib-Ala-Phe-Lys*3-PyrS2-3Thi-BztA-GlnR*3-Val-Thr- |
| | [mPEG8]-Lys-NH2 |
| I-1820 | Ac-PL3-Asp-Npg-B5-Asp-3COOHF-Aib-Ala-Phe-Lys*3-PyrS2-3Thi-BztA-GlnR*3-Leu-Ser- |
| | [mPEG8]-Lys-NH2 |
| I-1821 | Ac-PL3-Asp-Npg-B5-Asp-3COOHF-Aib-Ala-Phe-Lys*3-PyrS2-3Thi-BztA-GlnR*3-Leu-Ser- |
| | [mPEG8]-Lys-NH2 |
| I-1822 | Ac-PL3-Asp-Npg-B5-Asp-3COOHF-Aib-Ala-Phe-Lys*3-PyrS2-3Thi-BztA-GlnR*3-Aib-Ser- |
| | [mPEG8]-Lys-NH2 |
| I-1823 | Ac-PL3-Asp-Npg-B5-Asp-3COOHF-Aib-Ala-Phe-Lys*3-PyrS2-3Thi-BztA-GlnR*3-Aib-Ser- |
| | [mPEG8]-Lys-NH2 |
| I-1824 | Ac-PL3-Asp-Leu-B5-Asp-3COOHF-GlnMeBDA*3-Ala-Phe-Leu-PyrS2-Phe-34ClF-GlnR*3-NH2 |
| I-1825 | Ac-PL3-Asp-Leu-B5-Asp-3COOHF-GlnMeBDA*3-Ala-Phe-Ala-PyrS2-Phe-34ClF-GlnR*3-NH2 |
| I-1826 | Ac-PL3-Asp-Leu-B5-Asp-3COOHF-GlnMeBDA*3-Ala-Phe-Val-PyrS2-Phe-34ClF-GlnR*3-NH2 |
| I-1827 | Ac-PL3-Asp-Leu-B5-Asp-3COOHF-GlnR*3-Ala-Phe-Leu-PyrS2-Phe-34ClF-GlnMeBDA*3-NH2 |
| I-1828 | Ac-PL3-Asp-Leu-B5-Asp-3COOHF-GlnR*3-Ala-Phe-Ala-PyrS2-Phe-34ClF-GlnMeBDA*3-NH2 |
| I-1829 | Ac-PL3-Asp-Leu-B5-Asp-3COOHF-GlnR*3-Ala-Phe-Val-PyrS2-Phe-34ClF-GlnMeBDA*3-NH2 |
| I-1830 | Ac-PL3-Asp-Leu-B5-Asp-3COOHF-GlnR*3-Ala-Phe-Leu-PyrS2-Phe-34ClF-Gln5DA*3-NH2 |
| I-1831 | Ac-PL3-Asp-Leu-B5-Asp-3COOHF-GlnR*3-Ala-Phe-Ala-PyrS2-Phe-34ClF-Gln5DA*3-NH2 |
| I-1832 | Ac-PL3-Asp-Leu-B5-Asp-3COOHF-GlnR*3-Ala-Phe-Val-PyrS2-Phe-34ClF-Gln5DA*3-NH2 |
| I-1833 | Ac-PL3-Asp-Leu-B5-Asp-3COOHF-GlnR*3-Ala-Phe-Leu-PyrS2-Phe-34ClF-Gln6DA*3-NH2 |
| I-1834 | Ac-PL3-Asp-Leu-B5-Asp-3COOHF-GlnR*3-Ala-Phe-Ala-PyrS2-Phe-34ClF-Gln6DA*3-NH2 |
| I-1835 | Ac-PL3-Asp-Leu-B5-Asp-3COOHF-GlnR*3-Ala-Phe-Val-PyrS2-Phe-34ClF-Gln6DA*3-NH2 |
| I-1836 | Ac-PL3-Asp-Leu-B5-Asp-3COOHF-GlnT4CyMe*3-Ala-Phe-Leu-PyrS2-Phe-34ClF-GlnR*3-NH2 |
| I-1837 | Ac-PL3-Asp-Leu-B5-Asp-3COOHF-GlnT4CyMe*3-Ala-Phe-Val-PyrS2-Phe-34ClF-GlnR*3-NH2 |
| I-1838 | Ac-PL3-Asp-Leu-B5-Asp-3COOHF-GlnR*3-Ala-Phe-Leu-PyrS2-Phe-34ClF-GlnT4CyMe*3-NH2 |
| I-1839 | Ac-PL3-Asp-Leu-B5-Asp-3COOHF-GlnR*3-Ala-Phe-Ala-PyrS2-Phe-34ClF-GlnT4CyMe*3-NH2 |
| I-1840 | Ac-PL3-Asp-Leu-B5-Asp-3COOHF-GlnR*3-Ala-Phe-Val-PyrS2-Phe-34ClF-GlnT4CyMe*3-NH2 |
| I-1841 | Ac-PL3-Asp-DipA-B5-Asp-3COOHF-Ala-Ala-Phe-Lys*3-PyrS2-Phe-BztA-GlnR*3-NH2 |
| I-1842 | Ac-PL3-Asp-DipA-B5-Asp-3COOHF-Ala-Ala-Phe-Lys*3-PyrS2-Phe-BztA-GlnR*3-NH2 |
| I-1843 | Ac-PL3-Asp-DipA-B5-Asp-3COOHF-Ala-Ala-Phe-Lys*3-PyrS2-Phe-BztA-GlnR*3-Pro-NH2 |
| I-1844 | Ac-PL3-Asp-DipA-B5-Asp-3COOHF-Ala-Ala-Phe-Lys*3-PyrS2-Phe-BztA-GlnR*3-dPro-NH2 |
| I-1845 | Ac-PL3-Asp-DipA-B5-Asp-3COOHF-Ala-Ala-Phe-Lys*3-PyrS2-Phe-BztA-GlnR*3-dPro-NH2 |
| I-1846 | Ac-PL3-Asp-DipA-B5-Asp-3COOHF-Ala-Ala-Phe-Lys*3-PyrS2-Phe-BztA-GlnR*3-Ser-NH2 |
| I-1847 | Ac-PL3-Asp-DipA-B5-Asp-3COOHF-Ala-Ala-Phe-Lys*3-PyrS2-Phe-BztA-GlnR*3-Val-NH2 |
| I-1848 | Ac-PL3-Asp-DipA-B5-Asp-3COOHF-Ala-Ala-Phe-Lys*3-PyrS2-Phe-BztA-GlnR*3-dVal-NH2 |
| I-1849 | Ac-PL3-Asp-DipA-B5-Asp-3COOHF-Ala-Ala-Phe-Lys*3-PyrS2-Phe-BztA-GlnR*3-dVal-NH2 |
| I-1850 | Ac-PL3-Asp-DipA-B5-Asp-3COOHF-Leu-Ala-Phe-Lys*3-PyrS2-Phe-BztA-GlnR*3-NH2 |
| I-1851 | Ac-PL3-Asp-DipA-B5-Asp-3COOHF-Leu-Ala-Phe-Lys*3-PyrS2-Phe-BztA-GlnR*3-NH2 |
| I-1852 | Ac-PL3-Asp-DipA-B5-Asp-3COOHF-Val-Ala-Phe-Lys*3-PyrS2-Phe-BztA-GlnR*3-NH2 |
| I-1853 | Ac-PL3-Asp-DipA-B5-Asp-3COOHF-Thr-Ala-Phe-Lys*3-PyrS2-Phe-BztA-GlnR*3-NH2 |
| I-1854 | Ac-PL3-Asp-DipA-B5-Asp-3COOHF-Leu-Ala-Phe-Lys*3-PyrS2-Phe-BztA-GlnR*3-dPro-NH2 |
| I-1855 | Ac-PL3-Asp-DipA-B5-Asp-3COOHF-Val-Ala-Phe-Lys*3-PyrS2-Phe-BztA-GlnR*3-dPro-NH2 |
| I-1856 | Ac-PL3-Asp-DipA-B5-Asp-3COOHF-Thr-Ala-Phe-Lys*3-PyrS2-Phe-BztA-GlnR*3-dPro-NH2 |
| I-1857 | Ac-PL3-Asp-Ile-B5-Asp-3COOHF-Aib-Ala-Phe-sAla*3-PyrS2-Phe-34ClF-TriAzLys*3-NH2 |
| I-1858 | Ac-PL3-Asp-Ile-B5-Asp-3COOHF-Aib-Ala-Phe-TriAzLys*3-PyrS2-Phe-34ClF-sCH2S*3-NH2 |
| I-1859 | Ac-PL3-Asp-Ile-B5-Asp-3COOHF-Aib-Ala-Phe-sCH2S*3-PyrS2-Phe-34ClF-TriAzLys*3-NH2 |
| I-1860 | Ac-PL3-Asp-Ile-B5-Asp-3COOHF-Aib-Ala-Phe-TriAzOrn*3-PyrS2-Phe-34ClF-sCH2S*3-NH2 |
| I-1861 | Ac-PL3-Asp-Ile-B5-Asp-3COOHF-Aib-Ala-Phe-TriAzOrn*3-PyrS2-Phe-34ClF-sCH2S*3-NH2 |
| I-1862 | Ac-PL3-Asp-Ile-B5-Asp-3COOHF-Aib-Ala-Phe-sCH2S*3-PyrS2-Phe-34ClF-TriAzOrn*3-NH2 |
| I-1863 | Ac-PL3-Asp-Ile-B5-Asp-3COOHF-Ala-Ala-Phe-TriAzLys*3-PyrS2-Phe-34ClF-sAla*3-NH2 |
| I-1864 | Ac-PL3-Asp-Ile-B5-Asp-3COOHF-Ala-Ala-Phe-sAla*3-PyrS2-Phe-34ClF-TriAzLys*3-NH2 |
| I-1865 | Ac-PL3-Asp-Ile-B5-Asp-3COOHF-Ala-Ala-Phe-TriAzLys*3-PyrS2-Phe-34ClF-sCH2S*3-NH2 |
| I-1866 | Ac-PL3-Asp-Ile-B5-Asp-3COOHF-Ala-Ala-Phe-sCH2S*3-PyrS2-Phe-34ClF-TriAzLys*3-NH2 |
| I-1867 | Ac-PL3-Asp-Ile-B5-Asp-3COOHF-Ala-Ala-Phe-TriAzOrn*3-PyrS2-Phe-34ClF-sCH2S*3-NH2 |
| I-1868 | Ac-PL3-Asp-Ile-B5-Asp-3COOHF-Ala-Ala-Phe-TriAzOrn*3-PyrS2-Phe-34ClF-sCH2S*3-NH2 |
| I-1869 | Ac-PL3-Asp-Ile-B5-Asp-3COOHF-Ala-Ala-Phe-sCH2S*3-PyrS2-Phe-34ClF-TriAzOrn*3-NH2 |
| I-1870 | Ac-PL3-Asp-Ile-B5-Asp-3COOHF-Ala-Ala-Phe-sCH2S*3-PyrS2-Phe-34ClF-TriAzOrn*3-NH2 |
| I-1871 | Ac-S5-Asp-Asp-DipA-B5-Asp-3COOHF-Aib-Ala-Phe-Lys*3-PyrS1-3Thi-BztA-GlnR*3-NH2 |
| I-1872 | Ac-S5-Glu-Asp-DipA-B5-Asp-3COOHF-Aib-Ala-Phe-Lys*3-PyrS1-3Thi-BztA-GlnR*3-NH2 |
| I-1873 | Ac-S5-AcLys-Asp-DipA-B5-Asp-3COOHF-Aib-Ala-Phe-Lys*3-PyrS1-3Thi-BztA-GlnR*3-NH2 |
| I-1874 | Ac-S5-Leu-Asp-DipA-B5-Asp-3COOHF-Aib-Ala-Phe-Lys*3-PyrS1-3Thi-BztA-GlnR*3-NH2 |
| I-1875 | Ac-S5-3COOHF-Asp-DipA-B5-Asp-3COOHF-Aib-Ala-Phe-Lys*3-PyrS1-3Thi-BztA-GlnR*3-NH2 |
| I-1876 | Ac-S5-Ala-Asp-DipA-B5-Asp-3COOHF-Aib-Ala-Phe-Lys*3-PyrS1-3Thi-BztA-GlnR*3-NH2 |
| I-1877 | Ac-S5-Asp-Asp-Val-B5-Asp-3COOHF-Aib-Ala-Phe-Lys*3-PyrS1-3Thi-BztA-GlnR*3-NH2 |
| I-1878 | Ac-S5-Glu-Asp-Val-B5-Asp-3COOHF-Aib-Ala-Phe-Lys*3-PyrS1-3Thi-BztA-GlnR*3-NH2 |
| I-1879 | Ac-S5-AcLys-Asp-Val-B5-Asp-3COOHF-Aib-Ala-Phe-Lys*3-PyrS1-3Thi-BztA-GlnR*3-NH2 |
| I-1880 | Ac-S5-Leu-Asp-Val-B5-Asp-3COOHF-Aib-Ala-Phe-Lys*3-PyrS1-3Thi-BztA-GlnR*3-NH2 |
| I-1881 | Ac-S5-3COOHF-Asp-Val-B5-Asp-3COOHF-Aib-Ala-Phe-Lys*3-PyrS1-3Thi-BztA-GlnR*3-NH2 |
| I-1882 | Ac-S5-Ala-Asp-Val-B5-Asp-3COOHF-Aib-Ala-Phe-Lys*3-PyrS1-3Thi-BztA-GlnR*3-NH2 |
| I-1883 | Ac-Pro-S5-Ala-Asp-Leu-B5-Asp-3COOHF-Aib-Ala-Phe-Lys*3-PyrS1-Phe-34ClF-GlnR*3-NH2 |
| I-1884 | Ac-Pro-S5-Ala-Asp-Leu-B5-Asp-3COOHF-Aib-Ala-Phe-Lys*3-PyrS1-Phe-34ClF-GlnR*3-NH2 |
| I-1885 | Ac-Pro-S5-Glu-Asp-DipA-B5-Asp-3COOHF-Aib-Ala-Phe-Lys*3-PyrS1-Phe-34ClF-GlnR*3-NH2 |
| I-1886 | Ac-Pro-S5-Glu-Asp-Val-B5-Asp-3COOHF-Aib-Ala-Phe-Lys*3-PyrS1-Phe-34ClF-GlnR*3-NH2 |
| I-1887 | Ac-Pro-S5-Glu-Asp-Leu-B5-Asp-3COOHF-Aib-Ala-Phe-Lys*3-PyrS1-Phe-34ClF-GlnR*3-NH2 |
| I-1888 | Ac-Pro-S5-Ala-Asp-DipA-B5-Asp-3COOHF-Aib-Ala-Phe-Lys*3-PyrS1-Phe-34ClF-GlnR*3-NH2 |
| I-1889 | Ac-Pro-S5-Ala-Asp-Val-B5-Asp-3COOHF-Aib-Ala-Phe-Lys*3-PyrS1-Phe-34ClF-GlnR*3-NH2 |
| I-1890 | Ac-Pro-S5-Ala-Asp-DipA-B5-Asp-3COOHF-Aib-Ala-Phe-Lys*3-PyrS1-Phe-34ClF-GlnR*3-NH2 |
| I-1891 | Ac-Pro-S5-Val-Asp-Val-B5-Asp-3COOHF-Aib-Ala-Phe-Lys*3-PyrS1-Phe-34ClF-GlnR*3-NH2 |
| I-1892 | Ac-Pro-S5-Val-Asp-Leu-B5-Asp-3COOHF-Aib-Ala-Phe-Lys*3-PyrS1-Phe-34ClF-GlnR*3-NH2 |
| I-1893 | Ac-Pro-S5-Leu-Asp-DipA-B5-Asp-3COOHF-Aib-Ala-Phe-Lys*3-PyrS1-Phe-34ClF-GlnR*3-NH2 |
| I-1894 | Ac-Pro-S5-Leu-Asp-Val-B5-Asp-3COOHF-Aib-Ala-Phe-Lys*3-PyrS1-Phe-34ClF-GlnR*3-NH2 |
| I-1895 | Ac-Pro-S5-Leu-Asp-Leu-B5-Asp-3COOHF-Aib-Ala-Phe-Lys*3-PyrS1-Phe-34ClF-GlnR*3-NH2 |
| I-1896 | Ac-PL3-Asp-Leu-B5-Asp-3COOHF-GlnC4CyMe*3-Ala-Phe-Leu-PyrS2-Phe-34ClF-GlnR*3-NH2 |
| I-1897 | Ac-PL3-Asp-Leu-B5-Asp-3COOHF-GlnC4CyMe*3-Ala-Phe-Ala-PyrS2-Phe-34ClF-GlnR*3-NH2 |
| I-1898 | Ac-PL3-Asp-Leu-B5-Asp-3COOHF-GlnR*3-Ala-Phe-Leu-PyrS2-Phe-34ClF-GlnC4CyMe*3-NH2 |
| I-1899 | Ac-PL3-Asp-Leu-B5-Asp-3COOHF-GlnR*3-Ala-Phe-Ala-PyrS2-Phe-34ClF-GlnC4CyMe*3-NH2 |
| I-1900 | Ac-PL3-Asp-Leu-B5-Asp-3COOHF-Gln3ACPip*3-Ala-Phe-Ala-PyrS2-Phe-34ClF-GlnR*3-NH2 |
| I-1901 | Ac-PL3-Asp-Leu-B5-Asp-3COOHF-GlnR*3-Ala-Phe-Leu-PyrS2-Phe-34ClF-Gln3ACPip*3-NH2 |
| I-1902 | Ac-PL3-Asp-Leu-B5-Asp-3COOHF-GlnR*3-Ala-Phe-Ala-PyrS2-Phe-34ClF-Gln3ACPip*3-NH2 |
| I-1903 | Ac-PL3-Asp-Leu-B5-Asp-3COOHF-GlnPipAz*3-Ala-Phe-Leu-PyrS2-Phe-34ClF-GlnR*3-NH2 |
| I-1904 | Ac-PL3-Asp-Leu-B5-Asp-3COOHF-GlnPipAz*3-Ala-Phe-Ala-PyrS2-Phe-34ClF-GlnR*3-NH2 |
| I-1905 | Ac-PL3-Asp-Leu-B5-Asp-3COOHF-GlnR*3-Ala-Phe-Leu-PyrS2-Phe-34ClF-GlnPipAz*3-NH2 |
| I-1906 | Ac-PL3-Asp-Leu-B5-Asp-3COOHF-GlnR*3-Ala-Phe-Ala-PyrS2-Phe-34ClF-GlnPipAz*3-NH2 |
| I-1907 | Ac-PL3-Asp-Leu-B5-Asp-3COOHF-Aib-Ala-Phe-Lys*3-PyrS2-Phe-34ClF-GlnR*3-Ala-NH2 |
| I-1908 | Ac-PL3-Asp-Leu-B5-Asp-3COOHF-Aib-Ala-Phe-Lys*3-PyrS2-Trp-34ClF-GlnR*3-Ala-NH2 |
| I-1909 | Ac-PL3-Asp-Leu-B5-Asp-3COOHF-Aib-Ala-Phe-Lys*3-PyrS2-BztA-34ClF-GlnR*3-Ala-NH2 |
| I-1910 | Ac-PL3-Asp-Leu-B5-Asp-3COOHF-Aib-Ala-Phe-Lys*3-PyrS2-5ClW-34ClF-GlnR*3-Ala-NH2 |
| I-1911 | Ac-PL3-Asp-Leu-B5-Asp-3COOHF-Aib-Ala-Phe-Lys*3-PyrS2-6ClW-34ClF-GlnR*3-Ala-NH2 |
| I-1912 | Ac-PL3-Asp-Leu-B5-Asp-3COOHF-Aib-Ala-Phe-Lys*3-PyrS2-Phe-BztA-GlnR*3-Ala-NH2 |
| I-1913 | Ac-PL3-Asp-Leu-B5-Asp-3COOHF-Aib-Ala-Phe-Lys*3-PyrS2-Trp-BztA-GlnR*3-Ala-NH2 |
| I-1914 | Ac-PL3-Asp-Leu-B5-Asp-3COOHF-Aib-Ala-Phe-Lys*3-PyrS2-BztA-BztA-GlnR*3-Ala-NH2 |
| I-1915 | Ac-PL3-Asp-Leu-B5-Asp-3COOHF-Aib-Ala-Phe-Lys*3-PyrS2-5ClW-BztA-GlnR*3-Ala-NH2 |
| I-1916 | Ac-PL3-Asp-Leu-B5-Asp-3COOHF-Aib-Ala-Phe-Lys*3-PyrS2-6ClW-BztA-GlnR*3-Ala-NH2 |
| I-1917 | Ac-PL3-Asp-DipA-B5-Asp-3COOHF-Ala-Ala-Phe-Lys*3-PyrS2-3Thi-34ClF-GlnR*3-Ala-Ala- |
| | [AdamantC-dPEG2]-Lys-NH2 |
| I-1918 | Ac-PL3-Asp-DipA-B5-Asp-3COOHF-Ala-Ala-Phe-Lys*3-PyrS2-3Thi-34ClF-GlnR*3-Ala-Ala- |
| | [AdamantC-PEG8]--Lys-NH2 |
| I-1919 | Ac-PL3-Asp-DipA-B5-Asp-3COOHF-Ala-Ala-Phe-Lys*3-PyrS2-3Thi-34ClF-GlnR*3-Ala-Ala- |
| | [lithocholate-dPEG2]-Lys-NH2 |
| I-1920 | Ac-PL3-Asp-DipA-B5-Asp-3COOHF-Ala-Ala-Phe-Lys*3-PyrS2-3Thi-34ClF-GlnR*3-Ala-Ala- |
| | [lithocholate-PEG8]-Lys-NH2 |
| I-1921 | Ac-PL3-Asp-Leu-B5-Asp-3COOHF-Gln4Pippip*3-Ala-Phe-Leu-PyrS2-Phe-34ClF-GlnR*3-NH2 |
| I-1922 | Ac-PL3-Asp-Leu-B5-Asp-3COOHF-Gln4Pippip*3-Ala-Phe-Ala-PyrS2-Phe-34ClF-GlnR*3-NH2 |
| I-1923 | Ac-PL3-Asp-Leu-B5-Asp-3COOHF-GlnPip4AE*3-Ala-Phe-Ala-PyrS2-Phe-34ClF-GlnR*3-NH2 |
| I-1924 | Ac-PL3-Asp-Leu-B5-Asp-3COOHF-GlnR*3-Ala-Phe-Leu-PyrS2-Phe-34ClF-GlnPip4AE*3-NH2 |
| I-1925 | Ac-PL3-Asp-Leu-B5-Asp-3COOHF-GlnR*3-Ala-Phe-Val-PyrS2-Phe-34ClF-GlnPip4AE*3-NH2 |
| I-1926 | Ac-PL3-Asp-Leu-B5-Asp-3COOHF-GlnR*3-Ala-Phe-Ala-PyrS2-Phe-34ClF-GlnPip4AE*3-NH2 |
| I-1927 | Ac-S6-Glu-Asp-DipA-B5-Asp-3COOHF-Aib-Ala-Phe-Lys*3-PyrS1-Phe-34ClF-GlnR*3-NH2 |
| I-1928 | Ac-S6-Glu-Asp-DipA-B5-Asp-3COOHF-Aib-Ala-Phe-Lys*3-PyrS1-Phe-BztA-GlnR*3-NH2 |
| I-1929 | Ac-S6-Glu-Asp-DipA-B5-Asp-3COOHF-Aib-Ala-Phe-Lys*3-PyrS1-3Thi-BztA-GlnR*3-NH2 |
| I-1930 | Ac-S6-Glu-Asp-DipA-B5-Asp-3COOHF-Leu-Ala-Phe-Lys*3-PyrS1-Phe-34ClF-GlnR*3-NH2 |
| I-1931 | Ac-S6-Glu-Asp-DipA-B5-Asp-3COOHF-Leu-Ala-Phe-Lys*3-PyrS1-Phe-BztA-GlnR*3-NH2 |
| I-1932 | Ac-S6-Glu-Asp-DipA-B5-Asp-3COOHF-Leu-Ala-Phe-Lys*3-PyrS1-3Thi-BztA-GlnR*3-NH2 |
| I-1933 | Ac-S6-Glu-Asp-DipA-B5-Asp-3COOHF-Aib-Ala-Phe-Lys*3-PyrS1-Phe-34ClF-GlnR*3-Val-NH2 |
| I-1934 | Ac-S6-Glu-Asp-DipA-B5-Asp-3COOHF-Aib-Ala-Phe-Lys*3-PyrS1-3Thi-34ClF-GlnR*3-Val-NH2 |
| I-1935 | Ac-S5-Leu-Asp-Val-B5-Asp-3COOHF-Leu-Ala-Phe-Lys*3-PyrS1-3Thi-BztA-GlnR*3-NH2 |
| I-1936 | Ac-S5-Leu-Asp-Chg-B5-Asp-3COOHF-Leu-Ala-Phe-Lys*3-PyrS1-3Thi-BztA-GlnR*3-NH2 |
| I-1937 | Ac-S6-Leu-Asp-Val-B5-Asp-3COOHF-Leu-Ala-Phe-Lys*3-PyrS1-3Thi-BztA-GlnR*3-NH2 |
| I-1938 | Ac-S6-Leu-Asp-Val-B5-Asp-3COOHF-Aib-Ala-Phe-Lys*3-PyrS1-3Thi-BztA-GlnR*3-NH2 |
| I-1939 | Ac-S6-Leu-Asp-DipA-B5-Asp-3COOHF-Aib-Ala-Phe-Lys*3-PyrS1-3Thi-BztA-GlnR*3-NH2 |
| I-1940 | Ac-Pro-S6-Leu-Asp-Val-B5-Asp-3COOHF-Aib-Ala-Phe-Lys*3-PyrS1-3Thi-BztA-GlnR*3-NH2 |
| I-1941 | Ac-Pro-S6-Leu-Asp-Chg-B5-Asp-3COOHF-Aib-Ala-Phe-Lys*3-PyrS1-3Thi-BztA-GlnR*3-NH2 |

For agents described in the Tables, as described previously, in various embodiments N-terminal cap (N-Term) is connected via R₁ to the amino group (R₁) of the first amino acid (AA1). In some embodiments, a N-Term cap may be properly considered as part of AA1. From there, each carboxylate (R₂) of an amino acid is connected to the amino group (R₁) of the subsequent amino acid, until the carboxylate (R₂) of the final amino acid is connected to R₁ of a C-terminal group. For any amino acid that has a branch point (R₃) and a branching monomer is indicated in brackets, R₁ of the monomer in brackets is attached to R₃ of the amino acid. For the amino acid Dap, with two potential branch points (R₃ and R₄), if two branches are indicated, the R₁ of the first branch is connected to R₃, and R₁ of the second branch connected to R₄. For any pair of amino acids that terminate in a *3 designation, the Ra groups of each of those amino acids are linked to each other. Likewise, for any pair of amino acids that terminate in a **3 designation, the Ra groups of those amino acids are linked to each other. For any agent that contains a pair of branching amino acids with R₃ groups, and one contains a branching monomer that contains both R₁ and R₂ groups, then R₁ is attached to the branching amino acid adjacent to it in the sequence, and the R₂ group of the branching monomer is attached to R₃ of the amino acid with no branching monomer designated. For example, in various peptides that have one of Cys, hCys, Pen, or aMeC at position 10 and also one of Cys, hCys, Pen, or aMeC at position 14, and a branching group off of the amino acid residue 10, the R₁ of that branching group is tied to the R₃ of the amino acid residue at position 10, while the R₂ of that branching group is tied to the Ra of the amino acid residue at position 14. For any amino acid which has a branching amino acid containing R₃ and nothing attached to it by the above, then R₃=H. In various embodiments (e.g., agents described in Table E1, Table E2 and Table E3), PyrS2 is tied together with either R₄, R₅, R₆, or one arm of B5, and if PL3 is present, it is typically tied to the other arm of B5. In various embodiments, if a N-terminal group contains an olefin, it is tied to either AA3, or a branching group off of AA3. If a peptide has been reduced as indicated, then olefins have been hydrogenated to —CH₂—CH₂-after olefin metathesis; if it is indicated “C-term only”, then only the C-terminal side staple, e.g., in many cases PyrS2/R₅ olefin staple, has been hydrogenated to —CH₂—CH₂—. For peptides which have not been hydrogenated, two possible staple isomers can be generated for each olefin metathesis, leading to 2″ potential isomers (four if n=2). For peptides with the same description and different assigned numbers, these are separable isomers or compositions comprising one or more isomers. In various embodiments, for a peptide comprising an amino acid residue starting with “Dap7” or “DapAc7”, the olefin of that amino acid residue is tied together with one arm of B5 via olefin metathesis, while the R₃ group of that stapling amino acid residue is tied to the R₃ of another amino acid residue, e.g., GlnR*3 residue, elsewhere in the peptide. Special cases: For I-1484 and I-1485, PL3 is stapled to S5, while the R₅ residue is stapled to PyrS2.

In some embodiments, it was confirmed that various peptides, e.g., stapled peptides, comprising residues of amino acids described herein can provide higher affinity than reference peptides that comprise a reference amino acid, e.g., a natural amino acid such as Asp or Glu, but are otherwise identical.

### Example 3. Provided Technologies can Provide High Selectivity and Purity

Among other things, the present disclosure provides various technologies for preparing stapled peptides, including those comprising multiple staples. As described herein, in some embodiments, two or more staples are formed in one step. For example, in some embodiments, two or more staples are formed in a metathesis reaction. In some embodiments, all staples formed by metathesis are formed in a metathesis reaction. In some embodiments, each of such staples are formed through olefin metathesis of terminal olefins. In some embodiments, multiple staples are formed after full lengths of peptides have been achieved. In some embodiments, one or more staples comprising double bonds are formed after full lengths of peptides have been achieved. In some embodiments, all staples comprising double bonds are formed after full lengths of peptides have been achieved. In some embodiments, one or more staples formed through metathesis are formed after full lengths of peptides have been achieved. In some embodiments, all staples formed through metathesis are formed after full lengths of peptides have been achieved.

For example, in some embodiments, to prepare I-66 and I-67, a full length peptide (in some embodiments, prepared on solid phase as shown below) was subject to olefin metathesis:

In some embodiments, about 3:1 ratio (I-66: I-67) was observed.

In some embodiments, staples are formed in two or more staples. In some embodiments, two or more staples comprising olefin are formed in two or more steps. In some embodiments, two or more staples are formed in two or more metathesis steps. In some embodiments, two or more metathesis steps utilize different conditions, e.g., different catalysts. In some embodiments, each staple is formed in a separate step. In some embodiments, each staple comprising a double bond is formed in a separate step. In some embodiments, each staple comprising an olefin is formed in a separate step. In some embodiments, each staple formed by olefin metathesis is formed in a separate metathesis step. In some embodiments, stepwise stapling provides improved levels of selectivity to form a desired product (e.g., I-66) over other compounds, e.g., stereoisomers (e.g., for I-66, I-67). For example, in some embodiments, I-66 was prepared as described below, and over 10:1 I-66: I-67 ratio was observed. In some embodiments, the present disclosure provides a composition comprising I-66, wherein the ratio of I-66 to I-67 is about or at least about 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the present disclosure provides a composition comprising I-66 and I-67, wherein the ratio of I-66 to I-67 is about or at least about 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the ratio is about or at least about 5:1. In some embodiments, the ratio is about or at least about 10:1. In some embodiments, the ratio is about or at least about 20:1. In some embodiments, the ratio is about or at least about 30:1. In some embodiments, the ratio is about or at least about 50:1. In some embodiments, the ratio is about or at least about 80:1. In some embodiments, the ratio is about or at least about 90:1. In some embodiments, the ratio is about or at least about 100:1. In some embodiments, I-66 is provided in a salt form, e.g., a pharmaceutically acceptable salt form. In some embodiments, I-66 is provided in multiple forms including multiple salt forms. In some embodiments, I-67 is provided in a salt form, e.g., a pharmaceutically acceptable salt form. In some embodiments, I-67 is provided in multiple forms including multiple salt forms.

In a preparation, I-66 was synthesized by manual SPPS on Rink amide MBHA resin (98 g, 0.51 mmol/g loading, 50 mmol total). Deprotection steps were performed by treating the resin with 20% piperidine in DMF (v/v, 1000 mL) for thirty minutes with agitation via nitrogen bubbling. The resin was drained and washed with DMF four times. An amino acid to be coupled was dissolved in DMF (800 mL), and the coupling agent indicated below and either diisopropylethylamine (DIEA), or HOAt, were added in the equivalents listed below. Coupling proceeded for 30 minutes at room temperature with nitrogen bubbling, and the amino acid solution drained and the resin washed with DMF four times.

| Amino acid used | Coupling agent/base and amount used |
|---|---|
| Fmoc-Ala-OH (100 mmol) | HBTU (74 mmol), DIEA (75 mmol) |
| Fmoc-Glu(OAllyl)-OH (75 | DIC (75 mmol) and HOAt (75 mmol) |
| mmol) | |
| Fmoc-BztA-OH (65 mmol) | HBTU (61.5 mmol) and DIEA (65 mmol) |
| Fmoc-3Thi-OH (65 mmol) | HBTU (65 mmol) and DIEA (65 mmol) |
| Fmoc-PyrS2-OH (75 mmol) | HBTU (74 mmol), DIEA (75 mmol) |
| Fmoc-Lys(Alloc)-OH (100 | HATU (95 mmol) and DIEA (100 mmol) |
| mmol) | |
| Fmoc-Phe-OH (75 mmol) | HBTU (75 mmol), DIEA (75 mmol) |
| Fmoc-Ala-OH (90 mmol) | HBTU (85 mmol) and DIEA (90 mmol) |
| Fmoc-Aib-OH (100 mmol) | HBTU (95 mmol) and DIEA (100 mmol) |

After Aib addition, prior to Fmoc deprotection, the resin was washed with DMF five times, and dichloromethane five times. A solution of phenylsilane (54 g, 500 mmol) and tetrakis(triphenylphosphine) palladium (0) (5.77 g, 5 mmol) in dichloromethane (500 mL) was added. The reaction proceeded at room temperature for 15 minutes with nitrogen bubbling, and the palladium solution drained. The palladium/phenylsilane treatment was repeated another two times, then the resin drained and washed with DMF five times. The lactam was closed by treating the resin with HOAt (400 mmol) and DIC (400 mmol) in DMF (1000 mL), at room temperature with nitrogen bubbling for 2 h. The resin was drained and washed with DMF four times. The cycles of Fmoc deprotection and amino acid addition continued as above. A repeat coupling step was performed for Fmoc-Npg-OH.

| Amino acid used | Coupling agent/base and amount used |
|---|---|
| Fmoc-3COOHF(tBu)-OH | HATU (61.5 mmol), DIEA (65 mmol) |
| (65 mmol) | |
| Fmoc-Asp(tBu)-OH | HBTU (75 mmol) and DIEA (80 mmol) |
| (80 mmol) | |
| Fmoc-B5-OH (65 mmol) | HATU (61.5 mmol) and DIEA (65 mmol) |
| Fmoc-Npg-OH (75 mmol) | HATU (70 mmol) and DIEA (75 mmol) (×2) |
| (×2) | |
| Fmoc-Asp(tBu)-OH | HBTU (70 mmol) and DIEA (75 mmol) |
| (75 mmol) | |

After coupling Asp2, the B5/PyrS2 staple was closed by treating the resin with Hoveyda-Grubbs M720 catalyst (15.7 g, 25 mmol) and 1,4-benzoquinone (13.5 g, 125 mmol) in dichloroethane. The reaction proceeded at room temperature for 2 h with nitrogen bubbling, the catalyst was drained, and the treatment with M720 catalyst and 1,4-benzoquinone was repeated one more time before continuing with linear peptide synthesis.

| Amino acid/reagent used | Coupling agent/base and amount used |
|---|---|
| Fmoc-PL3-OH (75 mmol) | HBTU (75 mmol), DIC (75 mmol) |
| Ac2O (200 mmol) | DIEA (100 mmol) |

After N-terminal acetate capping, the PL3/B5 staple was closed by treating the resin with Grubbs catalyst M102 (20.6 g, 25 mmol) in dichloroethane at room temperature for 2 h with nitrogen bubbling. The catalyst solution was drained, and the treatment with Grubbs catalyst M102 was repeated another two times. The peptide was cleaved by treating the resin with 95:5 TFA: water (800 mL, v/v) for 2 hours, and the peptide was precipitated by pouring the cleavage cocktail into cold methyl tert-butyl ether. The precipitated peptide was filtered, washed with cold MTBE twice, and dried under vacuum. The peptide was first purified by dissolving in DMF, and loading onto a Luna C8 10 μm 100 Å column (flow rate: 20 mL/min) with a gradient of 45% to 75% acetonitrile in water (with 0.075% TFA) over 50 minutes. Product-containing fractions were dried, and the isolated peptide was subjected to a second purification, and was dissolved in 30% acetonitrile in water and loaded on a Kromasil C8 5 μm 100 Å column (20 mL/min), first flowing 0.4M ammonium acetate over the column for 25 min, then eluting with a gradient of 50% to 70% acetonitrile in water with 0.5% acetic acid over 50 minutes. The product-containing fractions were lyophilized to provide I-66 (40:1 I-66:1-67, 4997 mg, 2.41 mmol, 4.8% yield) plus a second lot of I-66 (8:1 I-66: I-67, 2015 mg, 0.97 mmol, 1.9% yield). Ratio of I-66 and I-67 were assessed using HPLC: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM; and ratio is calculated based on peak area. As an example, in one run, retention time of I-66 is 15.3 min and retention time of I-67 is 16.2 min. In some embodiments, such a protocol provides improved resolution compared to a reference protocol by which I-66 and I-67 may elute as one peak or may otherwise not sufficiently separated. For example, by the general method for Table E2 1-66 and I-67 can be eluted together as the second peak and the mixture may be designated as I-67). Alternatively or additionally, ratios can also be assessed using other technologies, e.g., NMR. In some embodiments, such a preparation of I-66 or preparations corresponding thereto were assessed in various biological assays and was confirmed to possess various properties and activities; see, e.g., Examples 11-18. ¹H NMR of such a preparation of I-66 is presented in FIG. 6. Certain additional NMR data are presented in FIG. 9 to FIG. 18. As those skilled in the art reading the present disclosure will appreciate, NMR spectra such as those in FIG. 6 and FIG. 9 to FIG. 18 may contain peaks of certain impurities and/or residue 1H in a NMR solvent. In some embodiments, NOE was observed between the peaks at about 5.45-5.6 and at about 5.2-5.35. Fractions can be further purified to provide improved purity.

In some embodiments, I-66 and/or I-67 prepared herein may be utilized as standard/reference to assess and/or characterize other compounds and/or other preparations of I-66 and/or I-67 (e.g., different batches prepared by the same or different methods). In some embodiments, I-470 is similarly prepared. In some embodiments, I-470 differs from I-66 in that I-470 has Glu2 and Glu5 while I-66 has Asp2 and Asp5. A preparation of I-470 is prepared as described below. In a preparation, I-470 was synthesized by manual SPPS on Rink amide MBHA resin (25.5 g, 0.51 mmol/g loading, 13 mmol total). Deprotection steps were performed by treating the resin with 20% piperidine in DMF (v/v, 200 mL) for thirty minutes with agitation via nitrogen bubbling. The resin was drained, and washed with DMF four times. An amino acid to be coupled was dissolved in DMF (800 mL), and a coupling agent indicated below and either diisopropylethylamine (DIEA), or HOAt, were added in the equivalents listed below. Coupling proceeded for 30 minutes at room temperature with nitrogen bubbling, and the amino acid solution drained and the resin washed with DMF four times.

| Amino acid used | Coupling agent/base and amount used |
|---|---|
| Fmoc-Ala-OH (26 mmol) | HOAt (26 mmol) and DIC (26 mmol) |
| Fmoc-Glu(OAllyl)-OH (19.5 mmol) | HBTU (18.5 mmol) and DIEA (19.5 mmol) |
| Fmoc-BztA-OH (16.9 mmol) | HBTU (16 mmol) and DIEA (19.5 mmol) |
| Fmoc-3Thi-OH (16.9 mmol) | HBTU (18.5 mmol) and DIEA (19.5 mmol) |
| Fmoc-PyrS2-OH (19.5 mmol) | HOAt (19.5 mmol) and DIC (19.5 mmol) |
| Fmoc-Lys(Alloc)-OH (26 mmol) | HATU (24.7 mmol) and DIEA (26 mmol) |
| Fmoc-Phe-OH (26 mmol) | HATU (24.7 mmol) and DIEA (26 mmol) |
| Fmoc-Ala-OH (26 mmol) | HBTU (24.7 mmol) and DIEA (26 mmol) |
| Fmoc-Aib-OH (26 mmol) | HOAt (26 mmol) and DIC (26 mmol) |

After Aib addition, prior to Fmoc deprotection, the resin was washed with DMF five times, and dichloromethane five times. A solution of phenylsilane (14 g, 130 mmol) and tetrakis(triphenylphosphine) palladium (0) (1.5 g, 1.3 mmol) in dichloromethane (100 mL) was added. The reaction proceeded at room temperature for 15 minutes with nitrogen bubbling, and the palladium solution drained. The palladium/phenylsilane treatment was repeated another two times, then the resin drained and washed with DMF five times. The lactam was closed by treating the resin with HOAt (104 mmol) and DIC (104 mmol) in DMF (100 mL), at room temperature with nitrogen bubbling for 2 h. The resin was drained and washed with DMF four times. The cycles of Fmoc deprotection and amino acid addition continued as above. A repeat coupling step was performed for Fmoc-Npg-OH.

| Amino acid used | Coupling agent/base and amount used |
|---|---|
| Fmoc-3COOHF(tBu)-OH (26 mmol) | HBTU (24.7 mmol) and DIEA (26 mmol) |
| Fmoc-Glu(tBu)-OH (26 mmol) | HBTU (24.7 mmol) and DIEA (26 mmol) |
| Fmoc-B5-OH (16.9 mmol) | HATU (15.7 mmol) and DIEA (16.9 mmol) |
| Fmoc-Npg-OH (19.5 mmol) (×2) | HATU (18.2 mmol) and DIEA (26 mmol) (×2) |
| Fmoc-Glu(tBu)-OH (26 mmol) | HBTU (24.7 mmol) and DIEA (26 mmol) |
| Fmoc-PL3-OH (16.9 mmol) | HATU (15.7 mmol) and DIC (16.9 mmol) |
| Ac2O (52 mmol) | DIEA (26 mmol) |

After N-terminal acetate capping, both staples were closed simultaneously by treating the resin with Grubbs catalyst M102 (5.25 g, 6.5 mmol) in dichloroethane at room temperature for 2 h with nitrogen bubbling. The catalyst solution was drained, and the treatment with Grubbs catalyst M102 was repeated another two times. The peptide was cleaved by treating the resin with 95:5 TFA: water (200 mL, v/v) for 2 hours, and the peptide was precipitated by pouring the cleavage cocktail into cold methyl tert-butyl ether. The precipitated peptide was filtered, washed with cold MTBE twice, and dried under vacuum. The peptide was first purified by dissolving in DMF, and loading onto a Luna C8 10 μm 100 Å column (flow rate: 20 mL/min) with a gradient of 0% to 80% acetonitrile in water (with 0.075% TFA) over 50 minutes. Product-containing fractions were lyophilized to give I-470 as a 3:1 mixture (3.4 g, 1.63 mmol, 12.5% yield).

In some embodiments, a preparation of the present disclosure is a mixture of trans: cis olefin isomers at a staple. In some embodiments, a preparation of the present disclosure is a mixture of trans: cis olefin isomers at a staple, e.g., a B5/PyrS2 staple.

In some embodiments, the present disclosure provides a compound having the structure of

or a salt thereof. In some embodiments, the present disclosure provides a compound having the structure of

or a salt thereof. In some embodiments, the present disclosure provides a compound having the structure of

or a salt thereof. In some embodiments, the present disclosure provides a compound having the structure of

or a salt thereof. In some embodiments, a compound has the same retention time as I-66 prepared above under the same or comparable HPLC conditions. For example, in some embodiments, a HPLC condition is Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM; and a retention time of I-66 is about 15.3 min. In some embodiments, a HPLC condition separates I-66 and I-67. In some embodiments, when co-injected with a I-66 preparation described herein, the compound elute as a single peak as I-66.

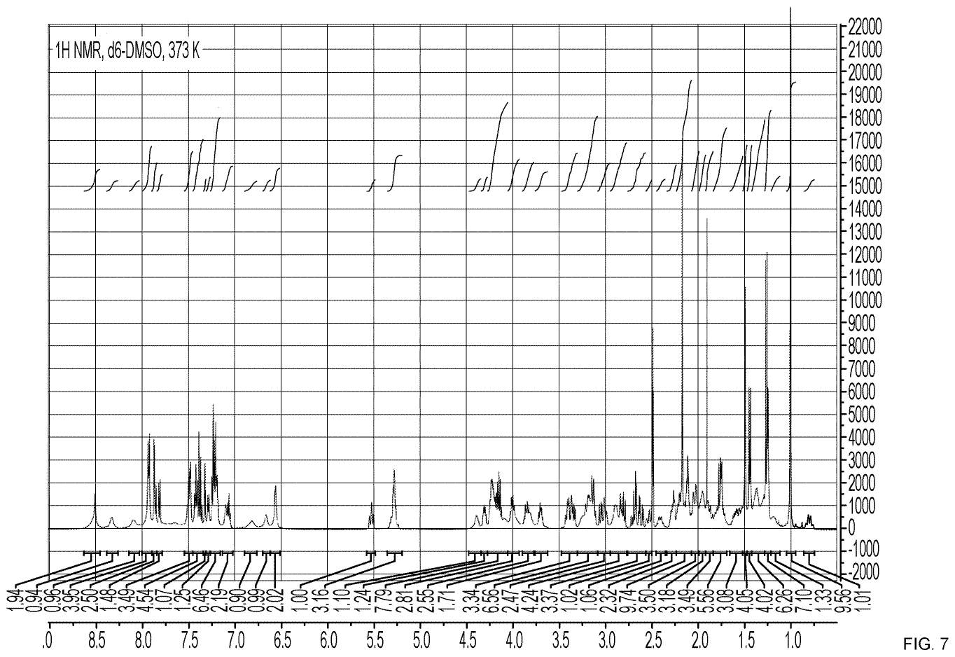

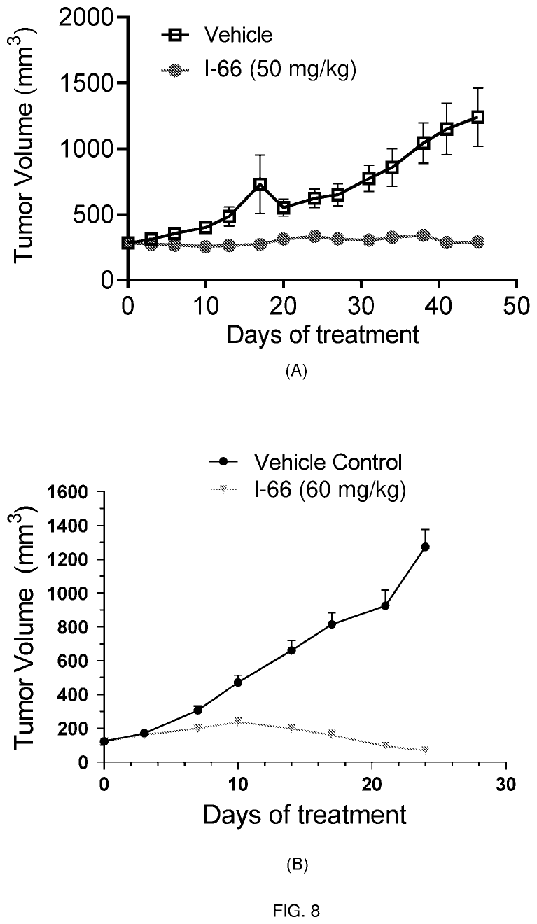

In some embodiments, the compound is characterized in that in its ¹H NMR spectrum, it shows peaks that overlap with those between about 5.1-5.7 in FIG. 6. In some embodiments, the compound is characterized in that in its ¹H NMR spectrum, it has the same peak pattern as FIG. 6 between about 5.1-5.7. In some embodiments, the compound has the same NMR spectra as I-66 under the same or comparable conditions. In some embodiments, the compound has the same ¹H NMR spectra as I-66 under the same or comparable conditions, e.g., DMSO-d6, 373 K. Those skilled in the art appreciate that peaks of certain 1H, such as those bonded to nitrogen and oxygen, may shift in ¹H NMR for the same compound during different assessments. In some embodiments, the compound has such a structure that for its ¹H NMR, peaks of 1H bonded to carbon are found in FIG. 6 under the same or comparable conditions (DMSO-d6, 373 K). In some embodiments, the compound has such a structure that its ¹H NMR peaks are found in FIG. 6 under the same or comparable conditions (DMSO-d6, 373 K). In some embodiments, the compound has such a structure that its ¹H NMR peaks for 1H bonded to carbon atoms are found in FIG. 6 under the same or comparable conditions (DMSO-d6, 373 K). In some embodiments, integration of peak(s) in FIG. 6 that correspond(s) to each 1H bonded to carbon in the compound is independently about 1 (e.g., about 0.1, 0.2, 0.3, 0.4, 0.5, 0.6, 0.7, 0.8, 0.9 to about 1.1, 1.2, 1.3, 1.4, 1.5, 1.6, 1.7, 1.8, 1.9 or 2.0, about 0.2-1.8, about 0.5-1.5, about 0.7-1.5, 0.8-1.2, etc.) when integration of the triplet at about 5.45 to about 5.6 is set as 1. In some embodiments, integration of peak(s) in FIG. 6 that correspond(s) to each 1H in the compound is independently about 1 (e.g., about 0.1, 0.2, 0.3, 0.4, 0.5, 0.6, 0.7, 0.8, 0.9 to about 1.1, 1.2, 1.3, 1.4, 1.5, 1.6, 1.7, 1.8, 1.9 or 2.0, about 0.2-1.8, about 0.5-1.5, about 0.7-1.5, 0.8-1.2, etc.) when integration of the triplet at about 5.45 to about 5.6 is set as 1. An integration of FIG. 6 is presented in FIG. 7 as an example.

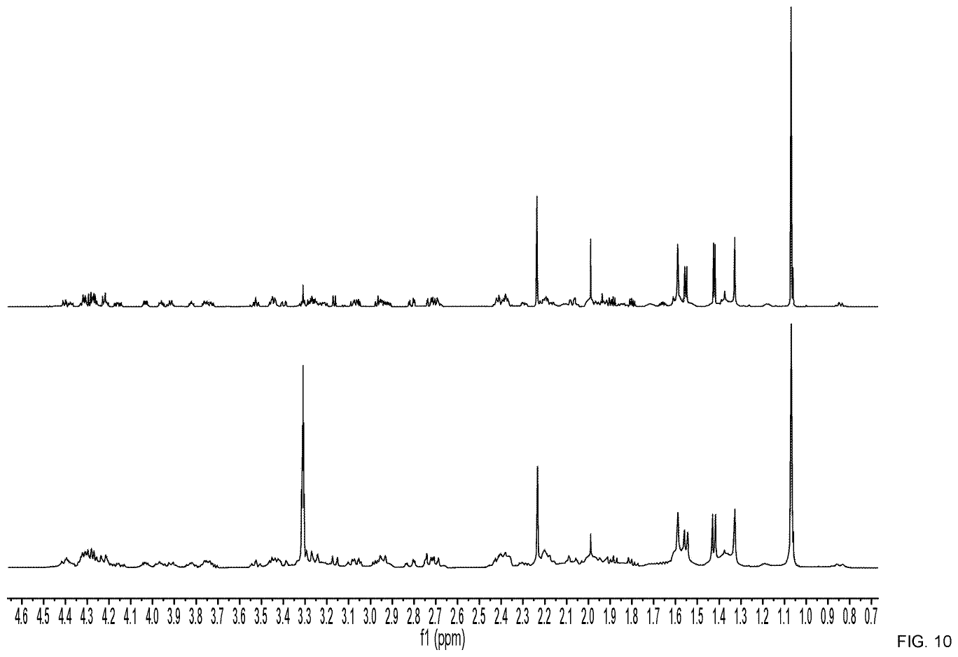

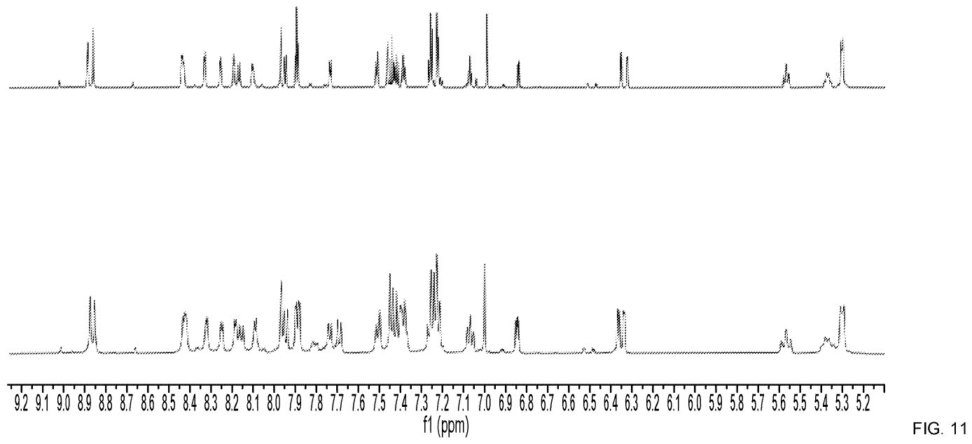

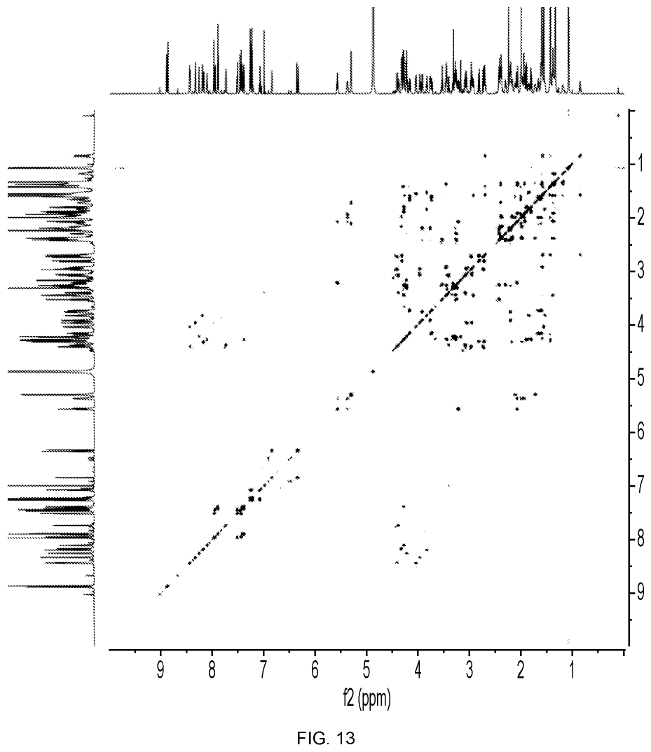

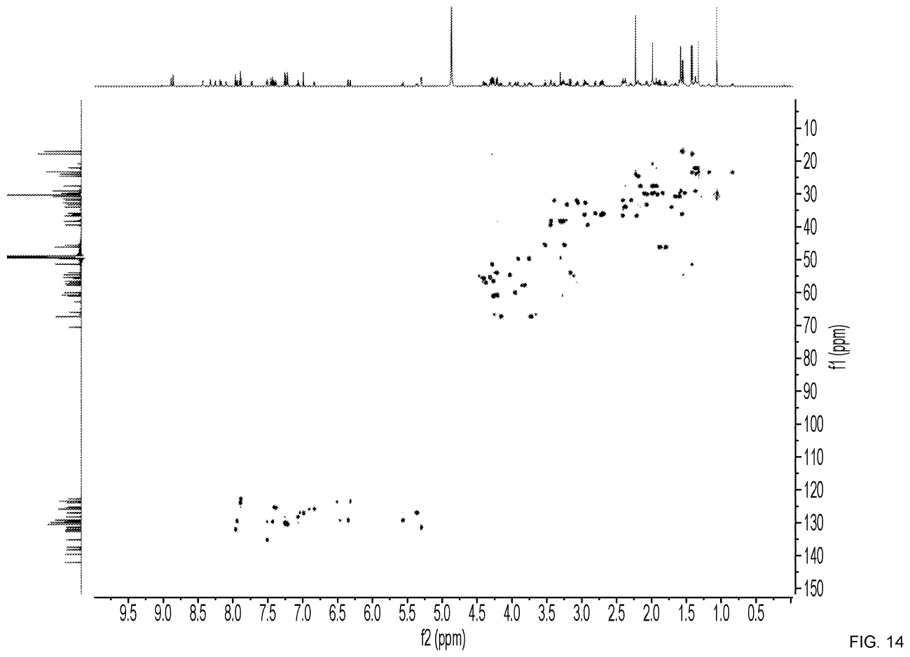

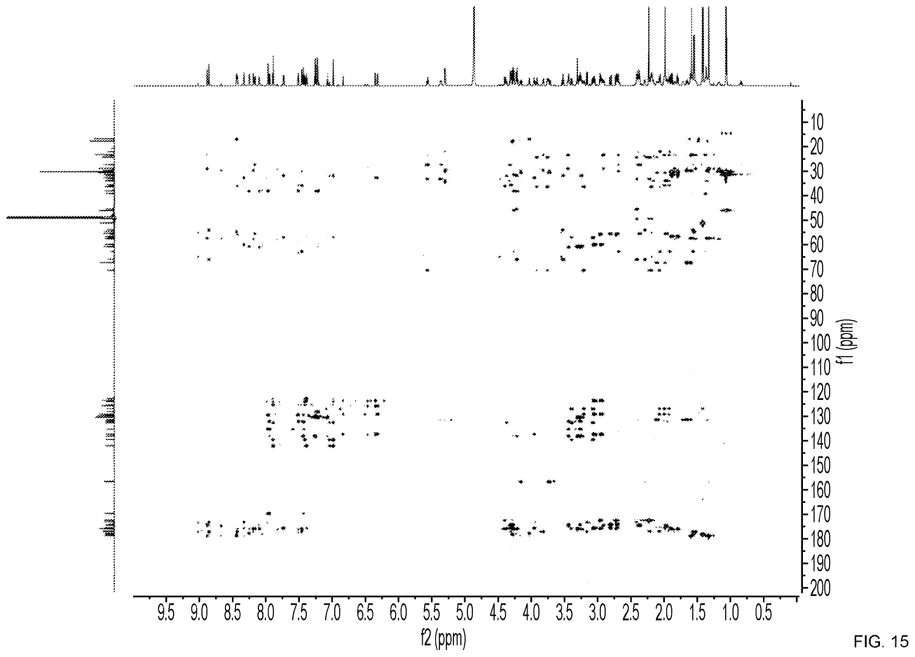

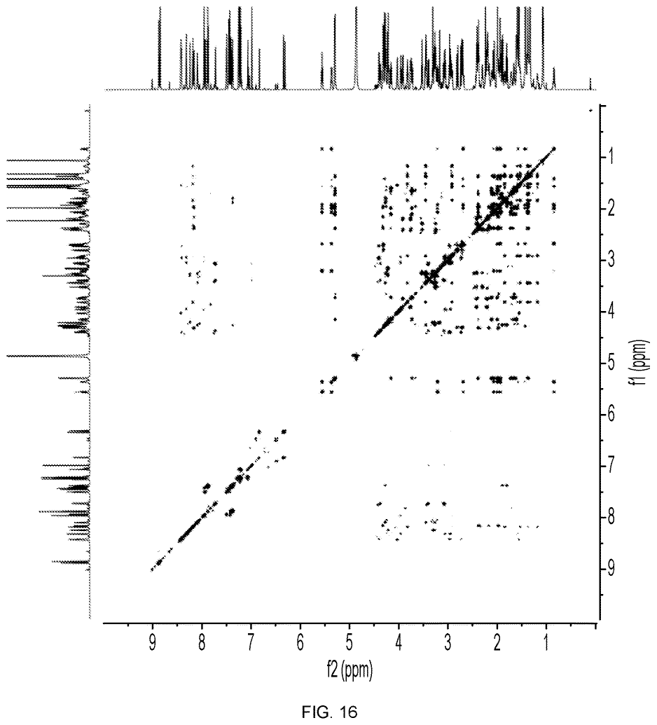

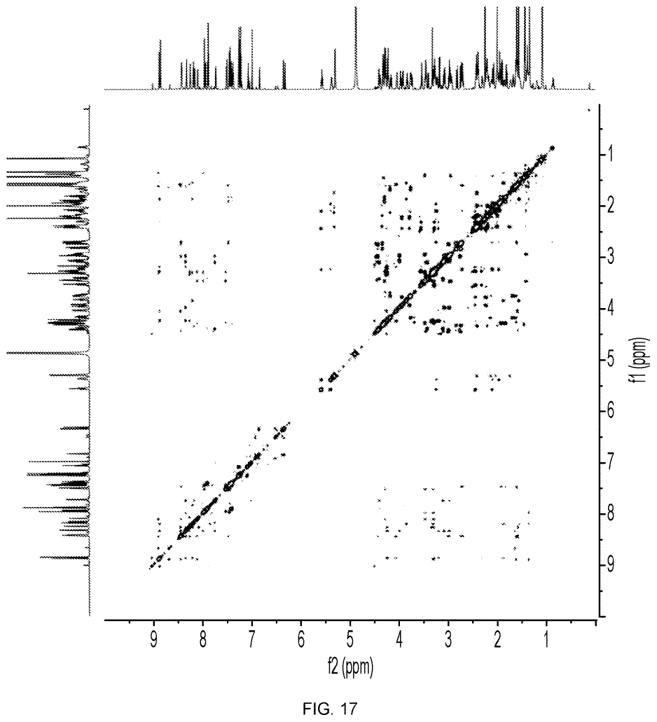

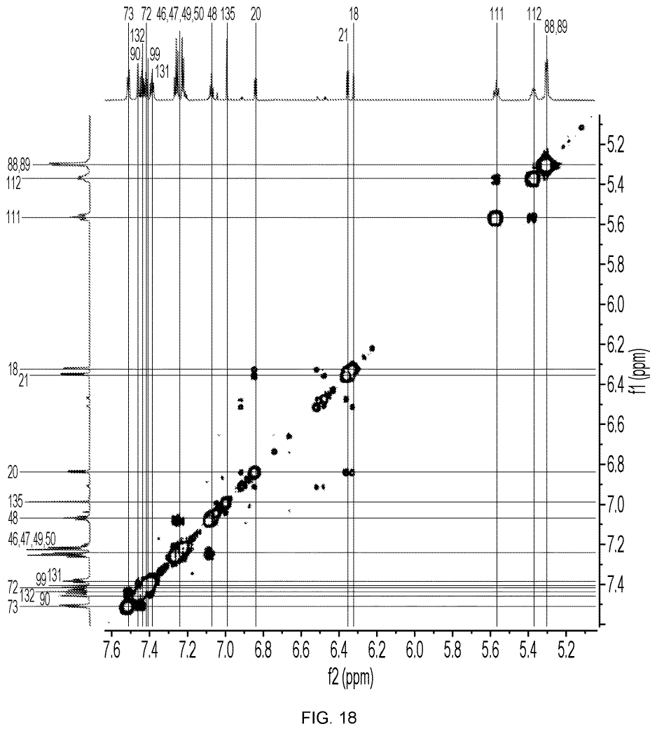

In some embodiments, the compound is characterized in that in its ¹H NMR spectrum, it shows peaks that overlap with those between about 5.1-5.7 in FIG. 9. In some embodiments, the compound is characterized in that in its ¹H NMR spectrum, it has the same peak pattern as FIG. 9 between about 5.1-5.7. In some embodiments, the compound has the same NMR spectra (e.g., one or more or all of FIG. 9, FIG. 10, FIG. 11, FIG. 12, FIG. 13, FIG. 14, FIG. 15, FIG. 16, FIG. 17, and FIG. 18) as I-66 under the same or comparable conditions, e.g., methanal-d4, 298 K. In some embodiments, the compound has the same ¹H NMR spectra (e.g., FIG. 9) as I-66 under the same or comparable conditions, e.g., methanal-d4, 298 K. In some embodiments, the compound has the same ¹³C NMR spectra (e.g., FIG. 12) as I-66 under the same or comparable conditions, e.g., methanal-d4, 298 K. In some embodiments, the compound has such a structure that for its ¹H NMR, peaks of 1H bonded to carbon are found in FIG. 9 under the same or comparable conditions (methanol-d4, 298 K). In some embodiments, the compound has such a structure that its ¹H NMR peaks are found in FIG. 9 under the same or comparable conditions (methanol-d4, 298 K). In some embodiments, the compound has such a structure that its ¹H NMR peaks for 1H bonded to carbon atoms are found in FIG. 9 under the same or comparable conditions (methanol-d4, 298 K). In some embodiments, the compound has such a structure that its ¹³C NMR peaks are found in FIG. 9 under the same or comparable conditions (methanol-d4, 298 K). In some embodiments, integration of peak(s) in FIG. 9 that correspond(s) to each 1H bonded to carbon in the compound is independently about 1 (e.g., about 0.1, 0.2, 0.3, 0.4, 0.5, 0.6, 0.7, 0.8, 0.9 to about 1.1, 1.2, 1.3, 1.4, 1.5, 1.6, 1.7, 1.8, 1.9 or 2.0, about 0.2-1.8, about 0.5-1.5, about 0.7-1.5, 0.8-1.2, etc.) when integration of the triplet at about 5.45 to about 5.6 is set as 1. In some embodiments, integration of peak(s) in FIG. 9 that correspond(s) to each 1H in the compound is independently about 1 (e.g., about 0.1, 0.2, 0.3, 0.4, 0.5, 0.6, 0.7, 0.8, 0.9 to about 1.1, 1.2, 1.3, 1.4, 1.5, 1.6, 1.7, 1.8, 1.9 or 2.0, about 0.2-1.8, about 0.5-1.5, about 0.7-1.5, 0.8-1.2, etc.) when integration of the triplet at about 5.45 to about 5.6 is set as 1. An integration of peaks in FIG. 9 is included in FIG. 9 as an example.

In some embodiments, ¹H NMR of a compound comprises the following peaks (or peaks corresponding to the following peaks) (methanol-d4, about 298K, about 900 MHz, chemical shift): 3.96, 6.34, 6.84, 6.36, 4.38, 4.31, 3.82, 4.21, 7.21, 7.24, 7.07, 7.24, 7.21, 4.03, 4.25, 7.97, 7.94, 7.42, 7.50, 4.40, 5.29, 5.31, 4.27, 4.31, 5.56, 5.36, 7.89, 7.38, 7.41, 7.89, 7.00, 4.30, 2.00, 2.10, 1.60, 1.65, 3.73, 4.15, 3.15, 4.22, 2.29, 2.41, 3.53, 3.25, 3.06, 2.95, 1.93, 2.38, 2.92, 3.45, 1.18, 1.36, 1.55, 1.84, 1.53, 3.27, 3.31, 3.27, 3.44, 2.74, 2.96, 1.92, 2.07, 1.37, 2.38, 1.80, 1.88, 2.71, 2.81, 3.20, 2.05, 1.97, 2.17, 0.84, 1.42, 1.57, 2.69, 3.76, 3.92, 2.20, 2.42, 2.22, 3.08, 3.40, 1.55, 1.07, 1.42, 1.59, 1.33, and 2.23. In some embodiments, chemical shifts of ¹H bonded to carbon of a compound are (methanol-d4, about 298K, about 900 MHz): 3.96, 6.34, 6.84, 6.36, 4.38, 4.31, 3.82, 4.21, 7.21, 7.24, 7.07, 7.24, 7.21, 4.03, 4.25, 7.97, 7.94, 7.42, 7.50, 4.40, 5.29, 5.31, 4.27, 4.31, 5.56, 5.36, 7.89, 7.38, 7.41, 7.89, 7.00, 4.30, 2.00, 2.10, 1.60, 1.65, 3.73, 4.15, 3.15, 4.22, 2.29, 2.41, 3.53, 3.25, 3.06, 2.95, 1.93, 2.38, 2.92, 3.45, 1.18, 1.36, 1.55, 1.84, 1.53, 3.27, 3.31, 3.27, 3.44, 2.74, 2.96, 1.92, 2.07, 1.37, 2.38, 1.80, 1.88, 2.71, 2.81, 3.20, 2.05, 1.97, 2.17, 0.84, 1.42, 1.57, 2.69, 3.76, 3.92, 2.20, 2.42, 2.22, 3.08, 3.40, 1.55, 1.07, 1.42, 1.59, 1.33, and 2.23. In some embodiments, 1H NMR of a compound comprises the following peaks (or peaks corresponding to the following peaks) (mcthanol-d4, about 298K, about 900 MHz, chemical shift): 8.32, 7.73, 8.17, 8.19, 8.25, 8.44, 8.89, 8.10, 8.42, 7.45, 7.39, 8.15, 8.86, and 7.69; in some embodiments, one or more or all such peaks independently correspond to 1H bonded to nitrogen and may shift or may not be present. In some embodiments, ¹³C NMR of a compound comprises the following peaks (or peaks corresponding to the following peaks) (methanol-d4, about 298K, about 226 MHz, chemical shift): 65.89, 137.36, 138.13, 57.28, 138.06, 132.12, 169.51, 62.78, 31.36, 70.51, 132.57, 139.58, 141.98, 156.51, 173.13, 175.55, 175.8, 174.47, 175.8, 177.56, 178.58, 174.39, 175.7, 177.0, 175.8, 175.44, 175.6, 176.97, 175.55, 178.08, 172.38, 59.94, 123.42, 125.19, 129.15, 56.93, 55.69, 57.70, 60.70, 130.39, 129.83, 128.08, 129.83, 130.39, 54.54, 60.94, 131.96, 129.37, 129.54, 135.08, 55.56, 131.26, 131.47, 56.48, 55.33, 129.2, 126.96, 122.71, 125.37, 125.20, 123.87, 126.99, 51.20, 29.99, 30.63, 67.18, 53.94, 31.81, 45.37, 32.60, 27.47, 33.84, 39.25, 23.2, 35.8, 29.61, 38.18, 38.10, 36.18, 30.00, 23.32, 33.8, 45.96, 35.69, 33.09, 27.46, 23.29, 35.96, 49.50, 24.46, 36.46, 31.76, 17.00, 30.18, 17.76, 28.98, 23.20, 23.87, 172.48, and 174.10. In some embodiments, chemical shifts of ¹³C of a compound are (methanol-d4, about 298K, about 226 MHz): 65.89, 137.36, 138.13, 57.28, 138.06, 132.12, 169.51, 62.78, 31.36, 70.51, 132.57, 139.58, 141.98, 156.51, 173.13, 175.55, 175.8, 174.47, 175.8, 177.56, 178.58, 174.39, 175.7, 177.0, 175.8, 175.44, 175.6, 176.97, 175.55, 178.08, 172.38, 59.94, 123.42, 125.19, 129.15, 56.93, 55.69, 57.70, 60.70, 130.39, 129.83, 128.08, 129.83, 130.39, 54.54, 60.94, 131.96, 129.37, 129.54, 135.08, 55.56, 131.26, 131.47, 56.48, 55.33, 129.2, 126.96, 122.71, 125.37, 125.20, 123.87, 126.99, 51.20, 29.99, 30.63, 67.18, 53.94, 31.81, 45.37, 32.60, 27.47, 33.84, 39.25, 23.2, 35.8, 29.61, 38.18, 38.10, 36.18, 30.00, 23.32, 33.8, 45.96, 35.69, 33.09, 27.46, 23.29, 35.96, 49.50, 24.46, 36.46, 31.76, 17.00, 30.18, 17.76, 28.98, 23.20, 23.87, 172.48, and 174.10. In some embodiments, for NMR performed in methanol-d4, the following chemical shift references are utilized: residual solvent protons: 3.31; solvent 13C: 49.00. In some embodiments, to collect NMR data, a preparation of I-66 was dissolved in methanol-d4, and NMR, e.g., 1H, 13C, COSY, TOCSY, NOESY, HSQC, HMBC, etc., data were acquired on an about 900 MHz instrument, e.g., a 900 MHz Bruker AVANCE III system with a cryoprobe. In some embodiments, sample concentration was about 10 mg/750 μL. In some embodiments, temperature is about 298 K. Certain results are presented in FIG. 9 to FIG. 18 as examples. In some embodiments, a strong NOE peak between the olefin protons of the PL3-B5 staple was observed. In some embodiments, this peak was stronger than the NOE peak between neighboring aromatic protons of 3Thi, and/or of the p and m protons in the phenyl group of Phe.

Those skilled in the art appreciate that NMR results such as ¹H NMR results (e.g., chemical shifts, integration of peaks, etc.), ¹³C NMR results, multidimensional (e.g., 2D) NMR results, etc., may have typical error ranges. In some embodiments, peaks corresponding to two or more 1H may overlap. In some embodiments, such peaks may be integrated together for assessment of numbers of 1H. In some embodiments, peaks corresponding to two or more 13C may overlap. In some embodiments, NMR of a preparation of I-66 described above are the same or comparable with or without the addition of the compound at a detectable level (e.g., the same amount of I-66) under the same or comparable conditions. In some embodiments, ¹H and/or ¹³C NMR of a preparation of I-66 described above are the same or comparable with or without the addition of the compound at a detectable level (e.g., the same amount of I-66, or about 0.1-10, 0.2-5, 0.5-2, 0.1, 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, or 10, etc. of I-66). In some embodiments, ¹H NMR are considered the same or comparable when peaks corresponding to 1H bonded to carbon have comparable chemical shift, peak shapes and/or integration. In some embodiments, ¹³C NMR are considered the same or comparable when peaks have comparable chemical shift. In some embodiments, peaks from impurities and solvents are properly excluded when comparing NMR. In some embodiments, peaks from impurities, solvents, 1H bonded to oxygen, nitrogen, etc. are properly excluded when comparing NMR. Those skilled in the art appreciate that spectra may be properly considered to be the same if those skilled in the art can reasonably conclude that such spectra are comparable to each other and are of the same compound.

In some embodiments, a compound has the same retention time as I-67 prepared above under the same or comparable HPLC conditions. For example, in some embodiments, a HPLC condition is Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM; and a retention time of I-67 is about 16.2 min. In some embodiments, a HPLC condition separates I-66 and I-67. In some embodiments, when co-injected with a I-67 preparation described herein, the compound elute as a single peak as I-67. In some embodiments, the compound has the same ¹H NMR peaks between about 5.0-6.0 as I-67 under the same or comparable conditions. In some embodiments, the compound has the same NMR spectra as I-67 under the same or comparable conditions. In some embodiments, the compound has the same 1H NMR spectra as I-67 under the same or comparable conditions, e.g., DMSO-d6, 373 K. In some embodiments, NMR of a preparation of I-67 described above are the same or comparable with or without the addition of the compound at a detectable level (e.g., the same amount of 1-67) under the same or comparable conditions. In some embodiments, ¹H NMR of a preparation of I-67 described above are the same or comparable with or without the addition of the compound at a detectable level (e.g., the same amount of I-67). In some embodiments, in a composition comprising the compound, ratio of the compound to a stereoisomer of the compound is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a composition comprising the compound, ratio of the compound to each stereoisomer of the compound is independently about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a composition comprising the compound, ratio of all compounds that are the compound or a salt thereof to all compounds that is a stereoisomer of the compound or a salt of the stereoisomer is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a composition comprising the compound, for each stereoisomer of the compound, ratio of all compounds that are the compound or a salt thereof to all compounds that is a stereoisomer of the compound or a salt of the stereoisomer is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the ratio is about or at least about 2:1. In some embodiments, the ratio is about or at least about 3:1. In some embodiments, the ratio is about or at least about 4:1. In some embodiments, the ratio is about or at least about 5:1. In some embodiments, the ratio is about or at least about 10:1. In some embodiments, the ratio is about or at least about 20:1. In some embodiments, the ratio is about or at least about 30:1. In some embodiments, the ratio is about or at least about 50:1. In some embodiments, the ratio is about or at least about 80:1. In some embodiments, the ratio is about or at least about 90:1. In some embodiments, the ratio is about or at least about 100:1.

In some embodiments, a preparation of I-66 comprises

or a salt thereof. In some embodiments, a preparation of I-67 comprises

or a salt thereof. In some embodiments, a preparation of I-66 or a preparation of I-67 comprises a first compound

or a salt thereof, and a second compound

or a salt thereof. In some embodiments, in a preparation of I-66, ratio of the first compound to the second compound is about or at least about 2:1, 3:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a preparation of I-66, ratio of all compounds that are the first compound or a salt thereof to all compounds that are the second compound or a salt thereof is about or at least about 2:1, 3:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a preparation of I-67, ratio of the second compound to the first compound is about or at least about 2:1, 3:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, in a preparation of I-67, ratio of all compounds that are the second compound or a salt thereof to all compounds that are the first compound or a salt thereof is about or at least about 2:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1, or 100:1. In some embodiments, the ratio is about or at least about 2:1. In some embodiments, the ratio is about or at least about 3:1. In some embodiments, the ratio is about or at least about 4:1. In some embodiments, the ratio is about or at least about 5:1. In some embodiments, the ratio is about or at least about 10:1. In some embodiments, the ratio is about or at least about 20:1. In some embodiments, the ratio is about or at least about 30:1. In some embodiments, the ratio is about or at least about 50:1. In some embodiments, the ratio is about or at least about 80:1. In some embodiments, the ratio is about or at least about 90:1. In some embodiments, the ratio is about or at least about 100:1. As utilized in the present disclosure, depending on the context, in some embodiments, a ratio is a molar ratio; in some embodiments, a ratio is a weight ratio; in some embodiments, a ratio is a volume ratio; and in some embodiments, a ratio is according to an assessment. For example, in some embodiments, when ratio of compounds are assessed using HPLC/UV, a ratio is of peak area of UV trace at a certain wavelength, e.g., 220 nm.

### Example 4. Provided Technologies can Provide High Selectivity

As confirmed below, in some embodiments, the present disclosure provides technologies with high selectivity for forming staples comprising olefin double bonds.

Fmoc-azidolysine-PyrS2-3Thi-BztA-propargylglycine-Ala-protide resin was synthesized using standard solid phase peptide synthesis procedures. The triazole staple was closed by treating the resin with one equivalent of copper (I) iodide, one equivalent of sodium ascorbate, ten equivalents of diisopropylethylamine, and ten equivalents of 2,6-lutidine in dichloromethane at room temperature for 48 h. The resin was washed for 5 min with DCM 2X, MeOH 1X, H₂O 2X, 50% H₂O/MeOH 2X, and MeOH 2X. In some embodiments, it was observed there was a small layer of insoluble material floating on top of the reactor, which was eliminated by aspiration through a hose connected to a pump. Then, continued with washes with NMP 2X, DCM 1X, and MeOH 1X.

The cyclized product was elongated to Fmoc-Asp(OtBu)—Npg-B5-Asp(OtBu)-3COOHF(OtBu)-Aib-Ala-Phe-TriAzLys*3-PyrS2-3Thi-Bzta-sAla*3-Ala-protide resin using standard solid phase peptide synthesis procedures. Afterwards, the resin was thoroughly washed with DCM 2X, NMP 1X, DCM 2X, MeOH 2X, DCM 1X, MeOH 1X, each for five minutes, then dried under a flow of nitrogen for 24 h to yield a gold color resin. The first staple was closed by treating the resin with 5 mol % Hoveyda-Grubbs M720 catalyst (CAS 301224-40-8) and 10 mol % benzoquinone in dichloromethane at reflux for 48 h. After 48 h, the catalyst solution was drained, the resin washed with dichloromethane 3X, dried, and then treated again with 5 mol % Hoveyda-Grubbs M720 catalyst (CAS 301224-40-8) and 10 mol % benzoquinone in dichloromethane at reflux for 48 h.

After analysis by LCMS, complete reaction was observed with no identifiable starting material. The desired product is detected in >95%, no other isomer by-product is observed. Double bond configuration is assigned based on analysis of NMR data, reported selectivity, etc., and can also be assessed by other technologies, e.g., crystallography.

The above product was elongated to Ac-PL3-Asp(OtBu)-Npg-B5-Asp(OtBu)-3COOHF(OtBu)-Aib-Ala-Phe-TriAzLys*3-PyrS2-3Thi-Bzta-sAla*3-Ala-protide resin using standard solid phase peptide synthesis procedures. After acetyl capping of the N-terminus, the resin was thoroughly washed with DCM 2X, NMP 1X, DCM 2X, MeOH 2X, DCM 1X, MeOH 1X, each for five minutes, then dried under a flow of nitrogen for 24 h. The second staple was closed by treating the resin with 30 mol % Grubbs I M102 (CAS 172222-30-9) and 60 mol % benzoquinone in dichloromethane at reflux for 24 h. After 24 h, the catalyst solution was drained, the resin washed with dichloromethane 3X, dried, and then treated again with 30 mol % Grubbs I M102 (CAS 172222-30-9) and 60 mol % benzoquinone in dichloromethane at reflux for 24 h. The crude product was cleaved and deprotected, and was analyzed by LCMS and showed 82% (UV at, e.g., 210-400 nm) of I-335. Two more peaks of olefin isomers were detected on as 13% and 5% of total area by HPLC, respectively. Double bond configuration is assigned based on analysis of NMR data, reported selectivity, etc., and can also be assessed by other technologies, e.g., crystallography.

### Example 5. Provided Technologies can Provide Various Advantages

Among other things, provided technologies can provide various advantages. In some embodiments, provided technologies can provide improved target binding profiles and/or activity profiles. For example, various stapled peptides, e.g., I-66, can provide strong binding to beta-catenin and modulation of gene expression.

In some embodiments, it was confirmed, e.g., through biochemical competition assays, that provided technologies (e.g., I-66) can inhibit TCF/LEF transcription factor binding to beta-catenin. In some embodiments, it was observed that provided technologies (e.g., I-66) compete with TCF1, TCF3, TCF4, LEF1, pAPC, mouse ECAD, human ECAD, etc. for beta-catenin interactions. In some embodiments, it was confirmed that provided technologies (e.g., I-66) can significantly reduce phospho-APC binding. In some embodiments, it was confirmed that provided technologies (e.g., I-66) can significantly reduce E-cadherin binding. In some embodiments, it was observed that there was little to no competitive effect for certain provided technologies, e.g., I-66, for ICAT, Axin or Bcl9. In some embodiments, interactions are dependent on phosphorylation, e.g., it has been reported that E-cadherin binding to beta-catenin is highly dependent on phosphorylation of up to eight Ser residues on E-cadherin.

In some embodiments, capabilities of provided technologies, e.g., binding to beta-catenin and/or disrupt its interactions (or lack thereof) with various partners were assessed and confirmed in cells, e.g., using a NanoBRET based assay in HEK293 cells. In some embodiments, it was observed that provided technologies, e.g., I-66, can potently inhibit such interactions without affecting cell viability.

Among other things, direct inhibition of endogenous beta-catenin/TCF interaction was confirmed by co-immunoprecipitation (co-IP) assays as described herein.

Among other things, the present disclosure confirms that provided technologies can inhibit transcription of endogenous Wnt pathway target genes driven by the beta-catenin/TCF interaction. Among other things, it was confirmed that in DLDI cells, peptide A and I-66 dose-dependently inhibited the expression of AXIN2 and SP5, two bona fide downstream genes of beta-catenin/TCF (peptide A: AXIN2 IC₅₀=9.3 uM, SP5 IC₅₀=9 uM; I-66: AXIN2 IC₅₀=1.6 uM, SP5 IC₅₀=1.3 uM). In some embodiments, no effect was observed on the expression of CTNNB1 for peptide A and I-66 in DLD1 cells under a tested condition. Reduction of expression level of a canonical beta-catenin target AXIN2, was also observed in COLO320DM cells (peptide A: IC₅₀=1.4 uM; I-66: IC₅₀=0.3 uM) while I-470 had no or very little or non-significant effect.

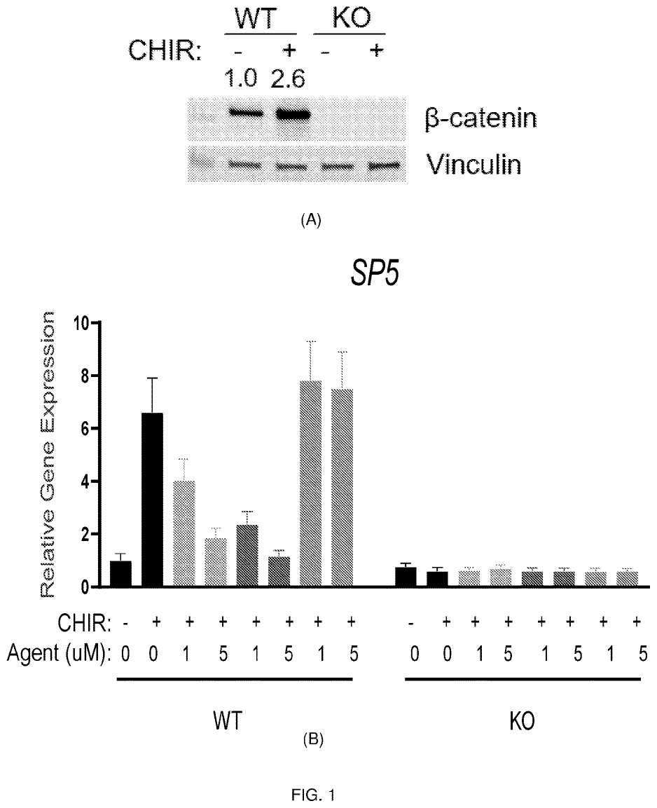

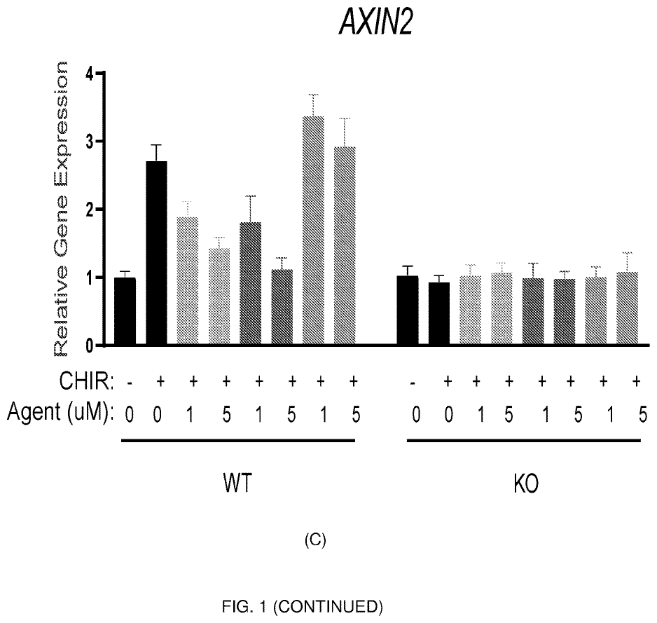

Among other things, provided technologies can modulate transcription and levels of various transcripts, in some embodiments, with selectivity. For example, in some embodiments, provided technologies inhibit beta-catenin driven transcription selectively in HAP1 WT cells. Certain data are presented in FIG. 1 as examples. In some embodiments, treatment with an agent, e.g., I-66, did not affect beta-catenin protein levels.

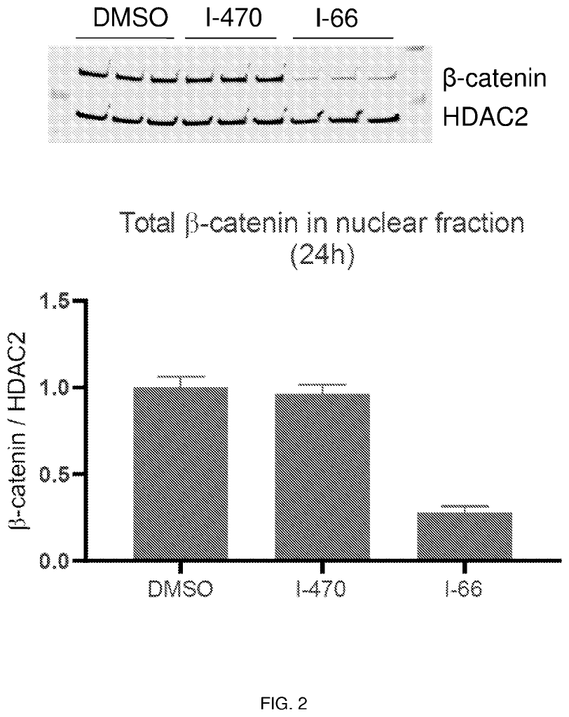

In some embodiments, provided technologies can reduce level of beta-catenin in nuclei. In some embodiments, provided technologies can block beta-catenin nuclear localization. In some embodiments, provided technologies can reduce level of beta-catenin nuclear translocation. For example, as confirmed in FIG. 2, provided technologies can reduce levels of nuclear beta-catenin in various cells including COLO320DM cells (10 uM, 24 hr).

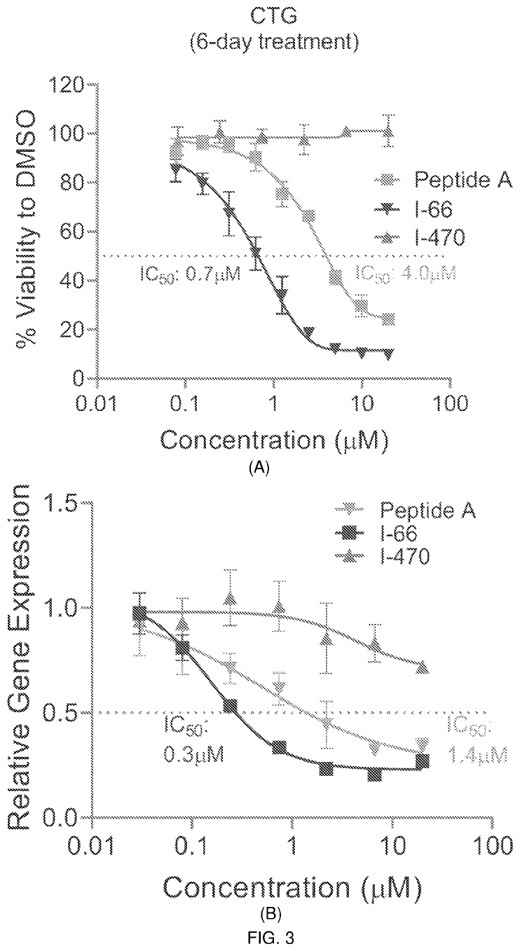

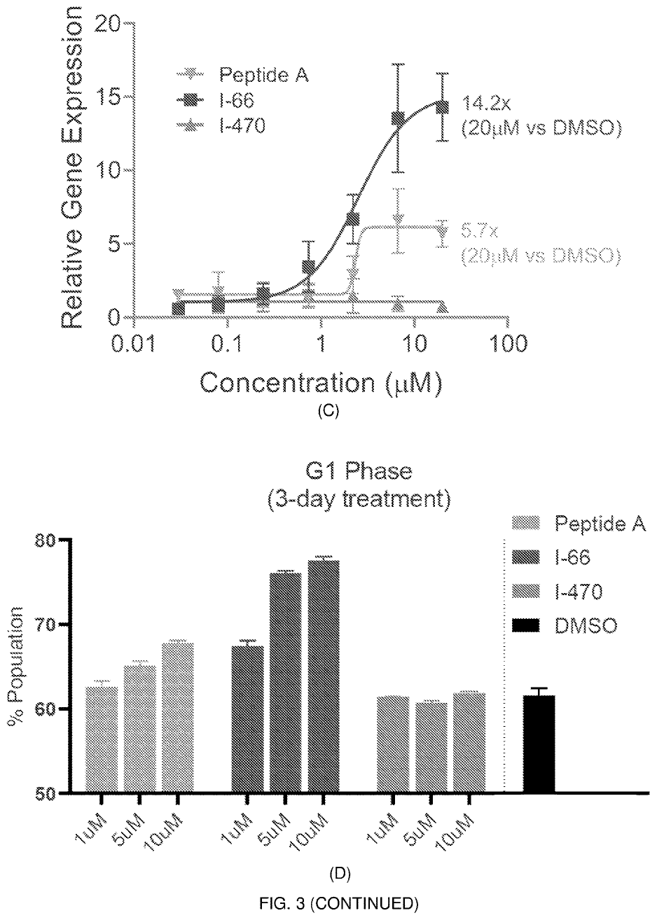

Among other things, provided technologies can inhibit proliferation of various cells including various cancer cells. In some embodiments, provided technologies modulate WNT specific transcription. In some embodiments, provided technologies induce cell cycle arrest. In some embodiments, provided technologies induce G1 cell cycle arrest. In some embodiments, provided technologies increased proportion of cells in G1 phase of cell cycle. As confirmed in FIG. 3, provided technologies can inhibit proliferation of COLO320DM, which is a colorectal cell line comprising various mutations such as APC, TP53, etc., modulate WNT specific transcription such as of AXIN2 and CXCL12, and induce G1 cell cycle arrest. In some embodiments, it was confirmed that provided technologies can reduce proportion of cells in S phase of cell cycle. In some embodiments, it was confirmed that provided technologies can significantly down-regulate Cyclin D2 and up-regulate p27. In some embodiments, changes of various genes, e.g., AXIN2, CXCL12, etc., were observed to be consistent with changes with shRNA-knockdown.

As described herein, provided technologies are useful for treating various conditions, disorders or diseases including cancer. Among other things, it was confirmed that provided technologies can provide in vivo efficacy as demonstrated in various animal models. Certain useful models and/or protocols are described below as examples. Those skilled in the art reading the present disclosure appreciate that various models for various cancers may be utilized to assess provided technologies and confirm their effects in accordance with the present disclosure.

COLO320DM human colorectal cancer cells (ATCC, CCL-220), which comprise various mutations, e.g., APC and TP53, etc., were expanded in RPMI 1640 media (10% FBS) and inoculated subcutaneously, 10⁷ cells per animal in 100 uL PBS/Matrigel (1:1) mixture, to male NU/J mice (JAX #2019) at 8 weeks of age. When the average tumor size reached 150 mm³, mice were randomized into 3 cohorts (n=10) and treated with vehicle (1% Tween 80/99% 10 mM PBS pH 7.4), I-66 (30 mg/kg), and I-66 (75 mg/kg) via intraperitoneal injection, once every 4 days for 5 doses.

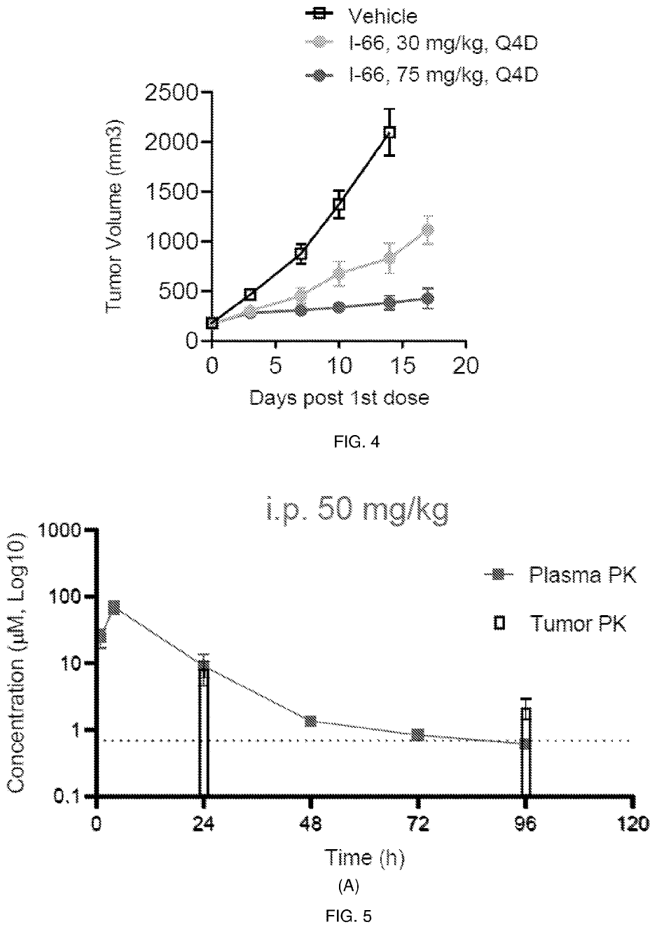

Tumor volume was measured by electronic caliper every 2-3 days until tumor volume reached 2000 mm³ and estimated as (length×width²)/2. Body weights were weighed every 2-3 days and represented as % body weight=(BWi−BW0)/BW0×100% (BWi: body weight at day i, BW0: body weight at day 0). Tumor growth inhibition was calculated as, TG1%=[1−(TVi−TV0)/(TVvi−TVv0)]×100% (TVi: average tumor volume of a dosing group on day i, TVO: average tumor volume of a dosing group on day 0, TVvi: average tumor volume of a vehicle group on day i, TVv0: average tumor volume of a vehicle group on day0). Animals were euthanized by CO₂ asphyxiation on the designated terminal day for each study, and plasma, tumors, tissues, etc., were excised for further analysis. Certain data are presented in FIG. 4 as examples.

As confirmed, technologies of the present disclosure can provide robust anti-tumor efficacy. For example, in some embodiments, in COLO320DM xenograft model, I-66 was dosed once every four days, and the treatment led to significant tumor growth inhibitions (TGI) of 66% and 89% at 30 and 75 mg/kg on day 14, respectively. At 75 mg/kg, an initial loss in body weight was observed after the first dose but recovered over time.

In some embodiments, transcriptional effects of pathway inhibition in vivo were assessed. For example, in some embodiments, several PD markers from COLO320DM tumors obtained at the end of the efficacy study (e.g., Day 18) were assessed. In agreement with in vitro and single-dose in vivo data, both AXIN2 and CXCL12 were dose-dependently regulated by provided technologies, e.g., I-66, in tumors (for AXIN2, down-regulation and for CXCL12, up-regulation), confirming durable target gene modulation. Reduction of mouse NOTUM level in plasma was also observed. In some embodiments, NOTUM may be utilized as a biomarker, e.g., for assessing a treatment, selecting patient population, determining whether to continue treatment, etc. In some embodiments, assessment of human plasma samples from normal and patients, e.g., colorectal cancer patients, confirms that NOTUM levels are correlated with stage of diseases and may be suitable for clinical applications, e.g., as a target engagement biomarker.

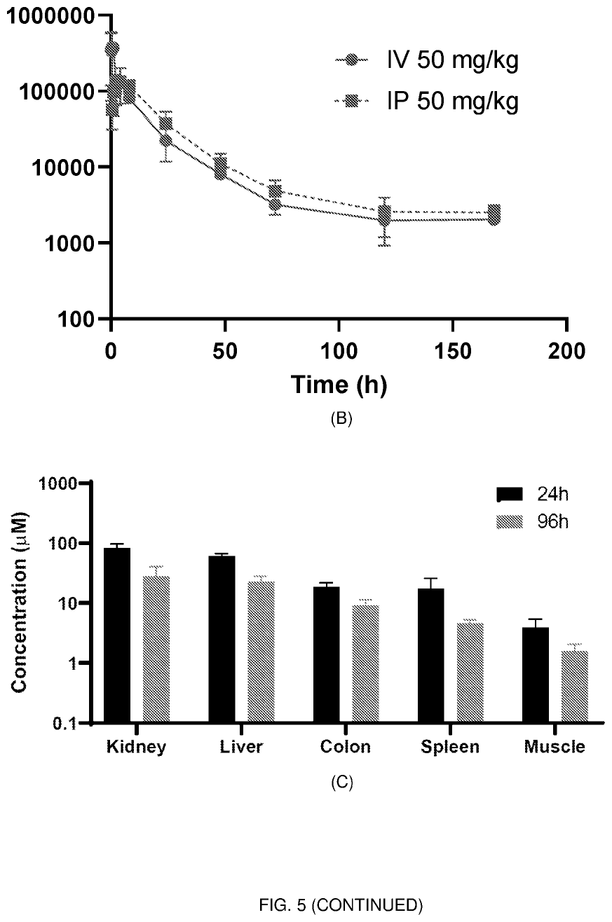

Among other things, various suitable in vivo pharmacokinetic and/or pharmacodynamic properties and/or activities have been confirmed. For example, as confirmed in FIG. 5, (A), provided technologies can be effectively delivered to tumors. As shown, prolonged tumor exposure to I-66 was observed after a single dose, and tumor exposure was about 2~10 fold above in vitro IC50 for proliferation. It was also observed that I-66 tumor PK exceeded plasma PK at 96 hr time point. Further, I-66 provided significantly longer time periods during which tumor exposure was about or above in vitro IC50 for proliferation when compared to, e.g., Peptide A (Ac-PL3-Asp-Npg-B5-Asp-3COOHF-Ala-Ala-Phe-Leu-PyrS2-2F3MeF-BztA-Gln-NH₂; PL3 and B5, and B5 and PyrS2 are stapled). A useful protocol for assessment is described below as an example.

Experiments were carried out under an Institutional Animal Care and Use Committee-approved protocol, and institutional guidelines for the proper and humane use of animals were followed.

For COLO320DM, male NU/J mice (6-8 weeks of age) were utilized, and mice were randomized when average tumor volume reached 300 mm³. For IP dosing, agents were formulated in 10 mg/mL arginine and 6% PEG400 phosphate (pH 7.4) formulation.

Concentrations of agents in biological samples were measured by LC-MS/MS (Triple Quad 6500+). Using analytical grade chemicals and solvents, 25 ng/ml Tolbutamide in acetonitrile (ACN, LS120-4, Fisher Scientific) was used as internal standards. 8 uL of plasma or tissue lysate was used for LC method with mobile phase A (1% formic acid (FA, LS118-4, Fisher Scientific) in H₂O) and mobile phase B (0.1% FA in ACN), 0.6 ml/min flow rate in Waters ACQUITY UPLC BEH C18 2.1*50 mm, 1.7 μm column. The calibration curve was generated using 5-5000 ng/ml agent, e.g., I-66, in mouse plasma and tissue homogenates. MS was conducted by electrospray ionization and multi reaction monitor scans. PK parameters such as plasma maximum concentration (Cmax), and AUC were analyzed by noncompartmental model 200 of Phoenix WinNonlin 8.3, using the linear/log trapezoidal method.

Additional data confirm well-behaved pharmacokinetic (PK) profiles of provided technologies. See, for example, FIG. 5, (B) and data below.

Certain PK parameters of I-66 in mouse by 2-compartmental analysis.

| PK Parameters | IV | IP |
|---|---|---|
| Cmax(ng/mL) | 498654 | 152000 |
| T_{1/2} (h) | 28.71 | 41.7 |
| Tₘₐₓ (h) | NA | 6.67 |
| Vdₛₛ (L/kg) | 0.452 | NA |
| Cl (mL/min/kg) | 0.30 | NA |
| Tₗₐₛₜ (h) | 168.00 | 168.00 |
| AUC₀₋ₗₐₛₜ (ng · h/mL) | 2741991 | 2971069 |
| AUC_{0-inf} (ng · h/mL) | 2826191 | 3124730 |
| Bioavailability (%) | NA | 105.0 |

In some embodiments, broad tissue distribution was observed. For example, as shown in FIG. 5, (C), I-66 was detected in all samples shown. In some embodiments, durable tissue residence was confirmed at least between 24- and 96-hr post-injection. In some embodiments, a single intraperitoneal (IP) dose of I-66 and I-470 at 100 mg/kg demonstrated comparable plasma AUC in mouse.

Robust and durable anti-tumor effects by provided technologies were confirmed in additional tumor models. In some embodiments, such effects were observed in a Patient-Derived Xenograft (PDX) cancer models.

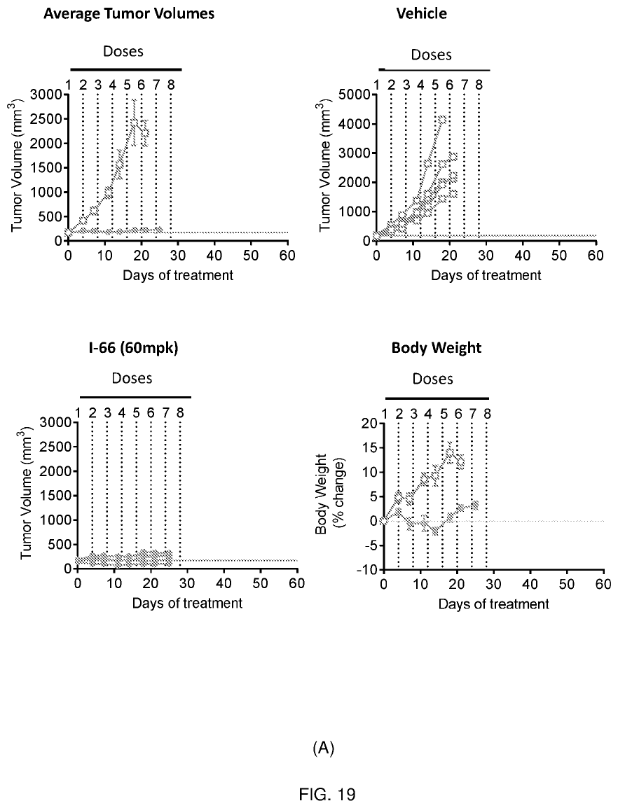

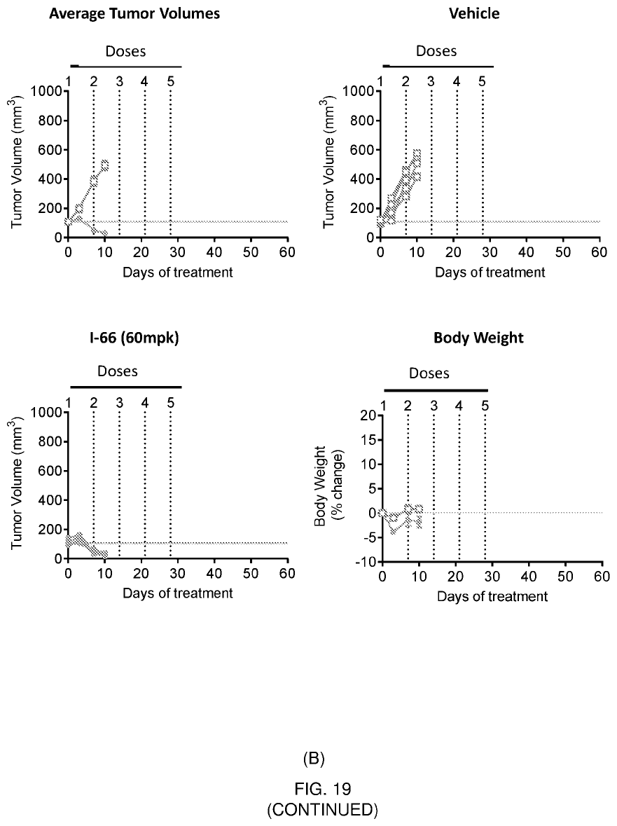

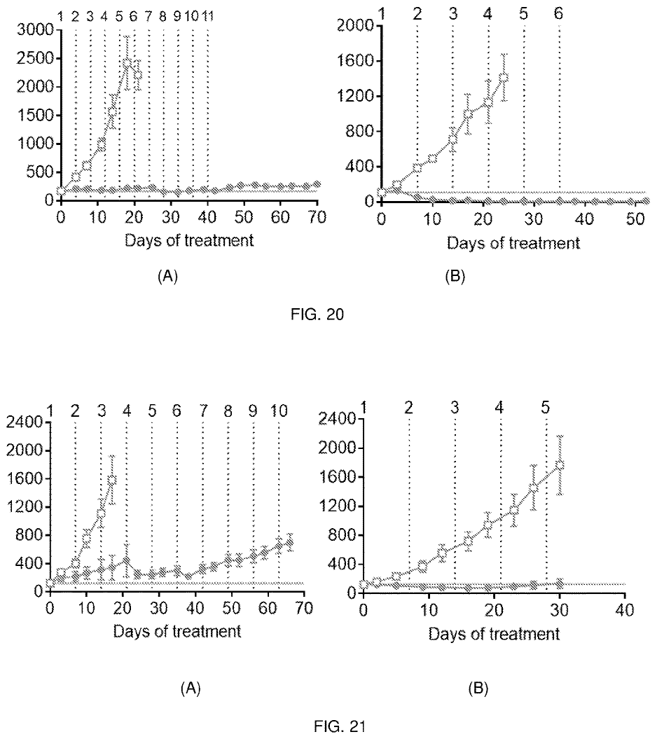

In some embodiments, provided technologies were assessed in hepatocellular cancer models. As shown in FIG. 19, provided technologies can effectively reduce tumor sizes. In some embodiments, tumors comprise beta-catenin mutations as described herein, e.g., beta-catenin D32H, D32V, etc. As confirmed in FIG. 20, in some embodiments, provided technologies can provide tumor volume reductions and/or reduce or prevent tumor growth across multiple doses and/or post doses (e.g., after about 10, 15, 20, 25, 30 or more days after a last dose). As confirmed in FIG. 21, provided technologies can be utilized against tumors comprising various beta-catenin mutations (e.g., S45F or S37C in FIG. 21, (A) and (B), respectively), in some embodiments with other mutations (e.g., MSH6 mutation in FIG. 21, (A)). In some embodiments, a tumor carries high mutation burden, e.g., as in FIG. 21, (A). In some embodiments, a tumor, e.g., as in FIG. 21, (A), carriers MSH6 mutation. In some embodiments, a tumor is MSI-H. Certain useful procedures are described herein as examples. In some embodiments, assessments were carried out under an Institutional Animal Care and Use Committee-approved protocol, and institutional guidelines for proper and humane use of animals were followed. I-66 were formulated in 10 mM sodium phosphate dibasic, 10 mg/mL arginine and 6% PEG400 phosphate (pH 7.4) formulation for IP dosing. Female BALB/c nude mice (6-8 weeks) were used. Mice were implanted subcutaneously at the right flank with frozen passage 5 (FP5) PDX tumor slices (~30 mm³ with Matrigel) for tumor development. The animals were randomized when tumors had grown to an approximate caliper-measured volume of about 108, 150 or 170 mm³. Mice were randomized into 2 dosing groups of 5 mice per group for vehicle and I-66 (60 mg/kg). Mice in each dosing group are injected once every 4 days (Q4D) or every 7 days (Q7D) via IP. Tumor volume was measured every 2-3 days using an electronic caliper until tumor volume reached 2000 mm³ as estimated by (length×width²)/2. Body weights were measured every 2-3 days.

Among other things, data in various Examples confirmed that provided technologies can provide robust PK properties, strong anti-tumor efficacy and on-target transcriptional modulation in vivo.

Among other things, provided technologies can modulate expression of various nucleic acids and/or levels of products thereof, e.g., RNA transcripts, polypeptides, etc. For example, tumor RNA-sequencing analysis confirmed that I-66 can provide, among other things, strong on-target Wnt/beta-catenin pathway modulation in COLO320DM tumors. Certain negatively enriched gene sets are presented below as examples. In some embodiments, a negatively enriched gene is CCND2, WNT5B, AXIN2, NKD1, WNT6, DKK1, or DKK4. In some embodiments, top Negatively Enriched Gene Sets include BCAT_GDS748_UP, BCAT.100_UP.V1_UP, HALLMARK_WNT_BETA-CATENIN_SIGNALING, RASHI-RESPONSE_TO_IONIZING-RADIATION_1, REACTOME-RRNA_PROCESSING, HALLMARK_MYC_TARGETS_V1, HALLMARK_MYC_TARGETS_V2, HALLMARK_OXIDATIVE_PHOSPHORYLATION, HALLMARK_E2F_TARGETS, HALLMARK_TNFA_SIGNALING_VIA_NFKB. I-66 vs. I-470. i.p. 30 mg/kg, 48 hr post single dose. NES-1.7 or smaller. FDR q-value 0.02 or smaller.

In some embodiments, structures, interactions, etc. are characterized and assessed using X-Ray crystallography and structure determination. In some embodiments, atomic coordinates and structure factors are deposited in the Protein Data Bank (e.g., for beta-catenin/I-66, 8EK2, etc.). Among other things, the structure confirmed that various amino acid residues in I-66 interact with various amino acid residues in beta-catenin, for example: PL3-1 with Val349, Asp2 with Lys312 and Gly307, Npg3 with Tyr306, Asp5 with Asn387 and Trp383, 3COOHF6 with Arg342 and Lys345, Ala8 with Trp383, Phe9 with Lys345 and Trp383, 3Thi12 with Trp383 and Asn415, and BztA13 with Gln379, Leu382, Val416, Asn415, and Trp383. Other residues, e.g., Q302, A305, V346, R386, N380, C419, etc., are also within vicinity of I-66 and may contribute to I-66 binding to beta-catenin.

In some embodiments, animal models are utilized to as provided technologies. Experiments were typically carried out under an Institutional Animal Care and Use Committee-approved protocol, and institutional guidelines for the proper and humane use of animals were followed.

Various useful technologies for assessing agents of the present disclosure and results therefrom are described in the Examples of WO 2022/261257 and are incorporated herein by reference.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described in the present disclosure. It is, therefore, to be understood that the foregoing embodiments are presented by way of example only and that, provided technologies, including those to be claimed, may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. A method for treating hepatocellular cancer in a subject, wherein the subject comprises hepatocellular cancer cells that comprise a D32H or D32V mutation in beta-catenin, comprising administering or delivering to the subject an effective amount of an agent having the structure of:
or a salt thereof.

2. A method for treating hepatocellular cancer in a subject, wherein the subject comprises hepatocellular cancer cells that comprise a S37C mutation in beta-catenin, comprising administering or delivering to the subject an effective amount of an agent described in claim 1.

3. A method for treating hepatocellular cancer in a subject, wherein the subject comprises hepatocellular cancer cells that comprise a S45F mutation in beta-catenin, comprising administering or delivering to the subject an effective amount of an agent described in claim 1.

4. A composition, characterized in that when the composition is administered to a hepatocellular tumor comprising a D32H, D32V, S37C or S45F mutation in beta-catenin, growth of the tumor is reduced, and wherein the composition comprises an agent having the structure of:
or a salt thereof.

5. A method for treating hepatocellular cancer in a subject, comprising administering or delivering to the subject an effective amount of:
an agent having the structure of:
or a salt thereof, wherein:
R_{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
a first R′ group and a second R′ group which are taken together to form -L^{s}- which is bonded to the atom to which a first R′ group is attached and the atom to which a second R′ group is attached; and
a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L^{s3}-, wherein each L^{s1}, L^{s2} and L^{s3} is independently L;
L^{AA1} is an amino acid residue that comprises a side chain comprising an acidic or polar group;
L^{AA2} is an amino acid residue that comprises a side chain comprising an acidic or polar group;
L^{AA3} is an amino acid residue;
L^{AA4} is an amino acid residue that comprises a side chain comprising an optionally substituted aromatic group;
L^{AA5} is an amino acid residue that comprises a side chain comprising an optionally substituted aromatic group;
L^{AA6} is an amino acid residue that comprises a side chain comprising an optionally substituted aromatic group;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms; or
an agent having the structure of formula I:
or a salt thereof, wherein:
R^{N} is a peptide, an amino protecting group or R′-L^{RN}-;
each of L^{P1}, L^{P2}, L^{P3}, L^{P4}, L^{P5}, L^{P6}, and L^{P7} is independently L, wherein L^{P1}, L^{P2}, L^{P3}, L^{P4}, L^{P5}, L^{P6}, and L^{P7} comprise:
a first R′ group and a second R′ group which are taken together to form -L^{s}- which is bonded to the atom to which a first R′ group is attached and the atom to which a second R′ group is attached; and
a third R′ group and a fourth R′ group which are taken together to form -L^{s}- which is bonded to the atom to which a third R′ group is attached and the atom to which a fourth R′ group is attached;
each L^{s} is independently -L^{s1}-L^{s2}-L83-, wherein each L^{s1}, L^{s2} and L^{s3} is independently L;
L^{AA1} is L^{AR}, wherein a methylene unit is replaced with —C(R′)(R^{AS})—, wherein R^{AS} is -L^{AS1}-R^{AA1}, wherein R^{AA1} is —CO₂R or —SO₂R;
L^{AA2} is L^{AR}, wherein a methylene unit is replaced with —C(R′)(R^{AS})—, wherein R^{AS} is -L^{AS2}-R^{AA2}, wherein R^{AA2} is —CO₂R, or —SO₂R;
L^{AA3} is L^{AR}, wherein a methylene unit is replaced with —C(R′)(R^{AS})—, wherein R^{AS} is -L^{AS3}-R^{AA3}, wherein R^{AA3} is R′;
L^{AA4} is L^{AR}, wherein a methylene unit is replaced with —C(R′)(R^{AS})—, wherein R^{AS} is -L^{AS4}-R^{AA4}, wherein R^{AA4} is an optionally substituted group selected from 6-14 membered aryl or 5-14 membered heteroaryl having 1-6 heteroatoms;
L^{AA5} is L^{AR}, wherein a methylene unit is replaced with —C(R′)(R^{AS})—, wherein R^{AS} is -L^{AS5}-R^{AA5}, wherein R^{AA5} is an optionally substituted group selected from 6-14 membered aryl or 5-14 membered heteroaryl having 1-6 heteroatoms;
L^{AA6} is L^{AR}, wherein a methylene unit is replaced with —C(R′)(R^{AS})—, wherein R^{AS} is -L^{AS6}-R^{AA6}, wherein R^{AA6} is an optionally substituted group selected from 6-14 membered aryl or 5-14 membered heteroaryl having 1-6 heteroatoms;
R^{C} is a peptide, a carboxyl protecting group, -L^{RC}-R′, —O-L^{RC}-R′ or —N(R′)-L^{RC}-R′;
each of L^{RN} and L^{RC} is independently L;
each L^{AR} is independently an optionally substituted, bivalent C₁-C₆ aliphatic group, wherein one or more methylene units of the group are optionally and independently replaced with —C(R′)₂—, —C(R′)(R^{AS})—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each of L^{AS1}, L^{AS2}, L^{AS3}, L^{AS4}, L^{AS5}, and L^{AS6} is independently L^{AS};
each R^{AS} is independently -L^{AS}-R′;
each L^{AS} is independently a covalent bond or an optionally substituted, bivalent C₁-C₁₀ aliphatic or heteroaliphatic group having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(0) 0-;
each -Cy- is independently an optionally substituted bivalent, 3-30 membered, monocyclic, bicyclic or polycyclic ring having 0-10 heteroatoms;
each R′ is independently -L-R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atom(s) to form an optionally substituted, 3-30 membered, monocyclic, bicyclic or polycyclic ring having, in addition to the intervening atom(s), 0-10 heteroatoms.

6. A method for treating hepatocellular cancer in a subject, comprising administering or delivering to the subject an effective amount of an agent having the structure of:
[X]ₚX¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇[X]p′,
wherein:
each of p15, p16 and p17 is independently 0 or 1;
each of p and p′ is independently 0-10; and
each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

7. The method of any one of claims 5-6, wherein the subject comprises hepatocellular cancer cells that comprise a mutation in beta-catenin.

8. The method of any one of claims 5-6, wherein the subject comprises hepatocellular cancer cells that comprise a D32H mutation in beta-catenin.

9. The method of any one of claims 5-6, wherein the subject comprises hepatocellular cancer cells that comprise a D32V mutation in beta-catenin.

10. The method of any one of claims 5-6, wherein the subject comprises hepatocellular cancer cells that comprise a S37C mutation in beta-catenin.

11. The method of any one of claims 5-6, wherein the subject comprises hepatocellular cancer cells that comprise a S45F mutation in beta-catenin.

12. A composition, characterized in that when the composition is administered to a hepatocellular tumor, growth of the tumor is reduced, and wherein the composition comprises an agent of claim 5.

13. A composition, characterized in that when the composition is administered to a hepatocellular tumor, growth of the tumor is reduced, and wherein the composition comprises an agent having the structure of:
wherein:
each of p15, p16 and p17 is independently 0 or 1;
each of p and p′ is independently 0-10; and
each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

14. The composition of any one of claims 12-13, wherein the tumor comprises hepatocellular cells comprising a mutation in beta-catenin.

15. The composition of claim 14, wherein the mutation is D32H.

16. The composition of claim 14, wherein the mutation is D32V.

17. The composition of claim 14, wherein the mutation is S37C.

18. The composition of claim 14, wherein the mutation is S45F.

19. The method or composition of any one of claims 6-18, wherein the agent is or comprises a peptide comprising:
wherein:
each of p0, p15, p16 and p17 is independently 0 or 1;
each of X⁰, X¹, X², X³, X⁴, X³, X⁶, X⁷, X⁸, X″, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group; and
each of X⁹, X¹² and X¹³ comprises a side chain comprising an optionally substituted aromatic group.

20. The method or composition of any one of claims 6-14, the agent comprises three or more staples within 10-20 amino acid residues.

21. The method or composition of any one of claims 6-20, wherein five of X⁰, X¹, X³, X⁴, X⁷, X¹⁰, X¹¹ and X¹⁴ are each independently an amino acid residue suitable for stapling, or are each independently stapled.

22. The method or composition of any one of claims 6-21, wherein X¹ and X⁴ are connected by a staple.

23. The method or composition of any one of claims 6-22, wherein X⁴ and X¹¹ are connected by a staple.

24. The method or composition of any one of claims 6-23, wherein X¹⁰ and X¹⁴ are connected by a staple.

25. The method or composition of any one of the preceding claims, wherein the agent comprises three or more staples.

26. The method or composition of any one of the preceding claims, wherein the agent comprises a N-terminal group.

27. The method or composition of any one of claims 6-26, wherein X¹ is PL3.

28. The method or composition of any one of claims 6-27, wherein X⁴ is a residue of an amino acid that comprises an olefin.

29. The method or composition of any one of claims 6-28, wherein X⁴ is B5.

30. The method or composition of any one of claims 6-29, wherein X¹⁰ is a residue of an amino acid that comprises an optionally substituted carboxyl group, an optionally substituted amino group, an azidyl group, an optionally substituted alkynyl group, or an optionally substituted thiol group.

31. The method or composition of any one of claims 6-30, wherein X¹⁰ is Lys.

32. The method or composition of any one of claims 6-31, wherein X¹¹ is a residue of an amino acid that comprises an olefin.

33. The method or composition of any one of claims 6-32, wherein X¹¹ is PyrS2.

34. The method or composition of any one of claims 6-33, wherein X¹⁴ is a residue of an amino acid that comprises a carboxyl group, an amino group, an azidyl group, an alkynyl group, or a thiol group.

35. The method or composition of any one of claims 6-34, wherein X¹⁴ is Glu.

36. The method or composition of any one of claims 6-35, wherein X¹⁰ and X¹⁴ are connected by a staple, wherein the staple comprises —C(O)N(R′)—.

37. The method or composition of any one of claims 6-36, wherein X² comprises a side chain comprising an acidic group.

38. The method or composition of any one of claims 6-37, wherein X² is Asp, Ala, Asn, Glu, Npg, Ser, Hse, Val, S5, S6, AcLys, TfeGA, aThr, Aad, Pro, Thr, Phe, Leu, PL3, Gln, isoGlu, MeAsn, isoDAsp, RbGlu, SbGlu, AspSH, Ile, SbMeAsp, RbMeAsp, aMeDAsp, OAsp, 3COOHF, NAsp, 3Thi, NGlu, isoDGlu, BztA, Tle, Aib, MePro, Chg, Cha, or DipA.

39. The method or composition of any one of claims 6-38, wherein X³ comprises one or two hydrophobic side chains.

40. The method or composition of any one of claims 6-39, wherein X³ is Npg, Ile, Asp, Cha, DipA, Chg, Leu, B5, Cba, S5, Ala, Glu, AllylGly, nLeu, Ser, B6, Asn, B4, GlnR, Val, [Phc][Allyl]Dap, Hse, [Bn][Allyl]Dap, 1MeK, R5, Phe, CypA, CyLeu, Pff, DiethA, Tyr, Trp, Aib, Phg, OctG, MorphNva, F2PipNva, [Piv][Allyl]Dap, [CyCO][Allyl]Dap, Lys, or S3.

41. The method or composition of any one of claims 6-40, wherein X⁵ comprises a side chain comprising an acidic group.

42. The method or composition of any one of claims 6-41, wherein X⁵ is selected from Asp, 3COOHF, TfeGA, Gln, [CH2CMe2CO2H]TriAzDap, Thr, Glu, 2OH3COOHF, 4OH3COOHF, 4COOHF, 2COOHF, His, Tyr, 5F3Me2COOHF, 4F3Me2COOHF, 5F3Me3COOHF, 4F3Me3COOHF, 3F2COOHF, Val, Ser, Trp, Asn, Ala, Arg, dGlu, aThr, hTyr, 3cbmf, Leu, Phe, Lys, and Ile.

43. The method or composition of any one of claims 6-42, wherein X⁶ comprises a side chain comprising an acidic group.

44. The method or composition of any one of claims 6-43, wherein X⁶ is 3COOHF, TfeGA, or Asp.

45. The method or composition of any one of claims 6-44, wherein X⁷ is a hydrophobic amino acid residue.

46. The method or composition of any one of claims 6-45, wherein X⁷ is selected from Aib, Ala, MorphGln, Gln, Ser, iPrLys, nLeu, Cha, Hse, Npg, Val, CyLeu, Thr, Phe, Acp, Asn, DaMeS, aMeDF, Leu, Cpg, Cbg, Me2Gln, Met20, AcLys, His, aMeL, DaMeL, aMeV, aMeS, and aMeF.

47. The method or composition of any one of claims 6-46, wherein X⁸ is a hydrophobic amino acid residue.

48. The method or composition of any one of claims 6-47, wherein X⁸ is selected from Ala, Aib, Cpg, Val, Leu, Gln, Lys, Asp, Glu, Aad, nLeu, Cba, Ser, Thr, aThr, MorphGln, Phe, hPhe, hTyr, and AcLys.

49. The method or composition of any one of claims 6-48, wherein X⁹ comprises a side chain which is or comprises an optionally substituted aromatic group.

50. The method or composition of any one of claims 6-49, wherein X⁹ is AA9, Phe, Ala, Lys, 3COOHF, Aib, 2NapA, nLeu, 2Thi, Tyr, 3Thi, 4FF, 4CIF, 4BrF, 3FF, 3CIF, 3BrF, 2FF, 30MeF, 4CNF, 3CNF, 4MeF, 3MeF, Aic, RbiPrF, SbiPrF, RbiPrDF, RbMeXylA, RbMeXyIDA, Cba, CypA, BztA, 1NapA, Trp, Leu, Ile, Ser, Chg, Hse, 4TriA, 3F3MeF, Thr, His, Val, Asn, Gln, 2Cpg, SbMeXylA, or SbMeXyIDA.

51. The method or composition of any one of claims 6-50, wherein X¹² comprises a side chain which is or comprises an optionally substituted aromatic group.

52. The method or composition of any one of claims 6-51, wherein X¹² is 3Thi, Phe, 2F3MeF, PyrS2, 2CIF, hnLeu, BztA, 2Thi, 2MeF, 2FF, 34CIF, Lys, nLeu, 2COOHF, 2PhF, hCbA, hCypA, hCha, CypA, hPhe, DipA, HepG, Dap7Abu, hhLeu, hhSer, HexG, [2IAPAc]2NH2F, Ala, Abu, Leu, hLeu, Npg, Cpa, PyrS1, [Bnc]2NH₂F, [Phc]2NH2F, [BiPh]2NH2F, [3PyAc]2NH2F, Nva, Cha, ChA, 2FurA, 2OMeF, 2BrF, 2CNF, 2NO2F, 2PyrA, 3PyrA, 4PyrA, His, 1NapA, Val, Ile, Chg, DiethA, OctG, 2cbmF, c6Phe, [McPipAc]2NH2F, or [2PyCypCO]2NH2F.

53. The method or composition of any one of claims 6-52, wherein the side chain of X¹³ comprises an optionally substituted aromatic group.

54. The method or composition of any one of claims 6-53, wherein X¹³ is selected from BztA, 34CIF, 2NapA, 3BrF, 34MeF, 3Thi, Phe, GlnR, 34MeF, 2NapA and Lys.

55. The method or composition of any one of claims 6-54, wherein p15 is 1.

56. The method or composition of any one of claims 6-55, wherein X¹⁵ comprises a hydrophobic side chain.

57. The method or composition of any one of claims 5-19, wherein the agent is an agent selected from Table E2 or Table E3, or the composition is a composition selected from Table E2 or Table E3.

58. The method or composition of any one of claims 6-56, wherein the peptide forms a structure that comprises a helix.

59. The method or composition of any one of claims 6-58, wherein a double bond of a (i, i+7) staple is E.

60. The method or composition of any one of claims 6-59, wherein a double bond of a (i, i+7) staple is Z.

61. The method or composition of any one of claims 6-60, wherein a double bond of a (i, i+2), (i, i+3) or (i, i+4) staple is E.

62. The method or composition of any one of claims 6-61, wherein a double bond of a (i, i+2), (i, i+3) or (i, i+4) staple is Z.

63. The method or composition of any one of claims 6-62, wherein a carbon atom bonded to two staples (e.g., in B5) is of R configuration.

64. The method or composition of any one of claims 6-62, wherein a carbon atom bonded to two staples (e.g., in B5) is of S configuration.

65. The method or composition of any one of claims 1-18, wherein the agent has the structure of SP-1-1, SP-1-2, SP-1-3, SP-1-4, SP-1-5, SP-1-6, SP-1-7, SP-1-8, SP-2-1, SP-2-2, SP-2-3, SP-2-4, SP-2-5, SP-2-6, SP-2-7, SP-2-8, SP-3-1, SP-3-2, SP-4-1, SP-4-2, SP-4-3, SP-4-4, SP-4-5, SP-4-6, SP-4-7, SP-4-8, SP-5-1, SP-5-2, SP-5-3, SP-5-4, SP-5-5, SP-5-6, SP-5-7, SP-5-8, SP-6, SP-7-1, SP-7-2, SP-7-3, SP-7-4, SP-7-5, SP-7-6, SP-7-7, SP-7-8, SP-8-1, SP-8-2, SP-8-3, SP-8-4, SP-8-5, SP-8-6, SP-8-7, SP-8-8, SP-9-1, SP-9-2, SP-9-3, SP-9-4, SP-9-5, SP-9-6, SP-9-7, SP-9-8, SP-10-1, SP-10-2, SP-10-3, SP-10-4, SP-10-5, SP-10-6, SP-10-7, SP-10-8, SP-11-1, SP-11-2, SP-11-3, SP-11-4, SP-11-5, SP-11-6, SP-11-7, SP-11-8, SP-12-1, SP-12-2, SP-12-3, SP-12-4, SP-12-5, SP-12-6, SP-12-7, SP-12-8, SP-13-1, SP-13-2, SP-13-3, SP-13-4, SP-13-5, SP- 13-6, SP-13-7, SP-13-8, SP-14-1, SP-14-2, SP-14-3, SP-14-4, SP-14-5, SP-14-6, SP-14-7, SP-14-8, SP-15-1, SP-15-2, SP-15-3, SP-15-4, SP-15-5, SP-15-6, SP-15-7, SP-15-8, or a salt thereof.

66. The method or composition of any one of claims 1-18, wherein the agent has the structure of
or a salt thereof.

67. The method or composition of claim 66, wherein the agent has the same retention time under a HPLC condition as I-66 prepared as described in Example 3, wherein the HPLC condition can separate I-66 and I-67 prepared as described in Example 3.

68. The method or composition of claim 66, wherein the agent shows a retention time of about 15.3 min under the following HPLC condition: Agilent Poroshell 120 EC-C18; 4.6×100 mm; solvent A=0.1% TFA in water; solvent B=0.075% TFA in acetonitrile; gradient is 10% B to 95% B over 30 min; detection is UV absorbance at 220 nM.

69. The method or composition of claim 48, wherein chemical shifts of 1H bonded to carbon of the agent are (methanol-d4, about 298K, about 900 MHz): 3.96, 6.34, 6.84, 6.36, 4.38, 4.31, 3.82, 4.21, 7.21, 7.24, 7.07, 7.24, 7.21, 4.03, 4.25, 7.97, 7.94, 7.42, 7.50, 4.40, 5.29, 5.31, 4.27, 4.31, 5.56, 5.36, 7.89, 7.38, 7.41, 7.89, 7.00, 4.30, 2.00, 2.10, 1.60, 1.65, 3.73, 4.15, 3.15, 4.22, 2.29, 2.41, 3.53, 3.25, 3.06, 2.95, 1.93, 2.38, 2.92, 3.45, 1.18, 1.36, 1.55, 1.84, 1.53, 3.27, 3.31, 3.27, 3.44, 2.74, 2.96, 1.92, 2.07, 1.37, 2.38, 1.80, 1.88, 2.71, 2.81, 3.20, 2.05, 1.97, 2.17, 0.84, 1.42, 1.57, 2.69, 3.76, 3.92, 2.20, 2.42, 2.22, 3.08, 3.40, 1.55, 1.07, 1.42, 1.59, 1.33, and 2.23.

70. The method or composition of claim 48, wherein chemical shifts of ¹³C of the agent are (methanol-d4, about 298K, about 226 MHz, chemical shift): 65.89, 137.36, 138.13, 57.28, 138.06, 132.12, 169.51, 62.78, 31.36, 70.51, 132.57, 139.58, 141.98, 156.51, 173.13, 175.55, 175.8, 174.47, 175.8, 177.56, 178.58, 174.39, 175.7, 177.0, 175.8, 175.44, 175.6, 176.97, 175.55, 178.08, 172.38, 59.94, 123.42, 125.19, 129.15, 56.93, 55.69, 57.70, 60.70, 130.39, 129.83, 128.08, 129.83, 130.39, 54.54, 60.94, 131.96, 129.37, 129.54, 135.08, 55.56, 131.26, 131.47, 56.48, 55.33, 129.2, 126.96, 122.71, 125.37, 125.20, 123.87, 126.99, 51.20, 29.99, 30.63, 67.18, 53.94, 31.81, 45.37, 32.60, 27.47, 33.84, 39.25, 23.2, 35.8, 29.61, 38.18, 38.10, 36.18, 30.00, 23.32, 33.8, 45.96, 35.69, 33.09, 27.46, 23.29, 35.96, 49.50, 24.46, 36.46, 31.76, 17.00, 30.18, 17.76, 28.98, 23.20, 23.87, 172.48, and 174.10.

71. The method or composition of any one of the preceding claims, wherein the agent has a diastercopurity of about 80%, 85%, 90%, 91%, 92%, 93%, 94%, 95%, 96%, 97%, 98%, 99% or more, or having a purity of about 80%, 85%, 90%, 91%, 92%, 93%, 94%, 95%, 96%, 97%, 98%, 99% or more.

72. The compositions of any one of claims 4 and 13-71, wherein growth of the tumor is reduced compared to a reference.

73. The composition of claim 72, wherein the reference is absence of administration of the agent.

74. The composition of claim 72, wherein the reference is administration of a reference agent.

75. The composition of claim 72, wherein the reference is administration of I-470.

76. The composition of any one of claims 4 and 13-75, wherein growth of the tumor is reduced in a mouse PDX model.

77. The composition of claim 76, wherein one or each dose administered to a mouse is independently about 20-80 mg/kg.

78. The composition of claim 76, wherein one or each dose administered to a mouse is independently about 60 mg/kg.

79. The composition of any one of claims 4 and 13-78, wherein growth of the tumor is reduced after about 1, 2, 3, 4, 5, 6, 7, 8, 9, or 10 doses.

80. A pharmaceutical composition, comprising or delivering an agent described in any one of the preceding claims, and a pharmaceutically acceptable carrier.

81. The method or composition of any one of the preceding claims, wherein the agent comprises one or more staples each independently comprises one or more olefin double bond, wherein the ratio of the two stereoisomers of an olefin double bond in a staple is about 2:1, 3:1, 4:1, 5:1, 6:1, 7:1, 8:1, 9:1, 10:1, 15:1, 20:1, 30:1, 40:1, 50:1 or more.

82. A method for inhibiting hepatocellular cancer cell proliferation, comprising administering or delivering to the hepatocellular cancer cell an effective amount of an agent or composition described in any one of the proceeding claims.

83. The method of any one of the any one of the preceding claims, comprising administering or deliver to a subject a second therapeutic agent or therapy.

84. The method of claim 83, wherein a second therapeutic agent is or comprises a chemotherapy agent, a hormone therapy agent, an immunotherapy agent, a checkpoint inhibitor, an antibody, a CTLA-4, PD-1 or PD-L1 inhibitor, or a cell, or a second therapy is or comprises surgery, chemotherapy, radiotherapy, hormone therapy, stem cell or bone marrow transplant, immunotherapy, T-cell therapy, or CAR T-cell therapy.

85. The method of any one of the preceding claims, comprising assessing expression of a nucleic acid.

86. An agent, compound, method, or composition of any one of Embodiments 1-520.
